SEQUENCE LISTING (140 sequences)

SEQ ID NO: 1 - protein, 51 aa, Unknown
  Viruses
MDYAIKPWWAARWETVEPEPEEPVYTDEETVYNEPTINDLIDMEMGHDYSR

SEQ ID NO: 2 - protein, 106 aa, Unknown
  Viruses
MNIENKLDVDAVLSEIIEDHDAFSENYDFDFSDYLKPIEIEDWVQDGKCQYRQCVYFSPKHNVHVAVNESRSGSYHSDWYYAVPTVELVELRERVVTQTVREWITL

SEQ ID NO: 3 - protein, 102 aa, Unknown
  Viruses
MIELNEQIIFLGDGTEGDLEYKLYEYMIWLAKAEGIDFVVSNPYGENTVVIGGTAYEVEWRYVGLKSEEYDVTDEGKWIPIGPWFWEHGEPDFEVSSWWCEK

SEQ ID NO: 4 - protein, 87 aa, Unknown
  Viruses
MFKVYGYDSNIHKCVYCDNAKRLLTVKKQPFEFINIMPEKGVFDDEKIAELLTKLGRDTQIGLTMPQVFAPDGSHIGGFDQLREYFK

SEQ ID NO: 5 - protein, 80 aa, Unknown
  Viruses
MTKRKEYMETAEKAVRELAIAYYNEHGKFPDRYSVLKSALTRSYKNMLSEVSDIIYKHKEQTGQSLDYDETFKQVLGIKE

SEQ ID NO: 6 - protein, 104 aa, Unknown
  Viruses
MKKRLLEDIAASSNSSLIKIIMAGEEDDLEMRGKIYGCDDYSPPVNWDSVMVMVERRERASKNVPNCPECGTEQVQLVHWQTNNLRYKCRHCKHRFDREENDKA

SEQ ID NO: 7 - protein, 308 aa, Unknown
  Viruses
MKTRKHYIDYFDSLITKHRDYQKGHREVINNILRDFLDYIGWENHICKDTQNAYSHSLGSLLEWFKRSRLLSSVIAVNNVKKFMYPSYIETNVSNDNVVTFNIINDVKRTYLEEWFSKDSKEKFASEFSHEFNNNVNMLFKHSRRLFCHGDDRTINVNVKDWVTAKFIPSSQNGPFELLIIVCAPHEIYKNLPYMKPCEANKHNKTIRSLTYKLRTLLSKMDVVESFDDNTNYGLSLFETKVVIKLKDPNKFKPTPKPNHGNDTMKEEREYLSTRLIEVEKQIEEHTKVLKDLTAKANGLRNAIEVLK

SEQ ID NO: 8 - protein, 333 aa, Unknown
  Viruses
MTRNEYIKSFNSVIDDKAIPMFGQNSVLSIINQWLNSVDASIVSSTKFIHEIRKISSRVDKDVIKKTFKESRLLSYLVNRDILGNFGKEIKRTKDVVGYNWFGDVNSYHLNNKEDPENIFTRRWISNFRLFKKQILKSASKLCYGDYRQIHPLASDMIIIKEYELDKNKVSIFVNYGFFTPETNQKNINKFFSIASTITRQLETALLCMETVENIHTYPFKNICGWEGYKLVISLREVKCAYSPTSKEIYQQKCDEIVNTPKEETTLEELMECLDDSPEPIEIRPEVIALEKAYKEVLEISNKAQKEYEQAKKIWEESVNRLDRLEQALQLIK

SEQ ID NO: 9 - protein, 340 aa, Unknown
  Viruses
MKTRSQIEDMVRNASYTRDVMTFLCENNLDPDKVNRVIHHFKYTNSSEWVRNFSKAGYITQMTAREQLTDFCKTIDYKNPLFVQGVGQSKVDLSSGFFNPNHYRIEWRFIALFRRQLKQILSTASRLKGSDINLKNLKFDGYTLQMEVRPLKENNRTARISFKPNTKNSLSICECLKSQLTEAFKYMDVVAAVQSKILPRFERFKLDTTSYELDMIVSFKYEFLRKDEVTQEKKQEVQDNLNLSNYLSNDPKFWMYSSGNKDAWKFNKVNFLPVENPSLKPVEKWHADAIEKSLKAVDAELVKATNEVLEAEKALEQAQSRVQNLTKQRSKLNNALNALN

SEQ ID NO: 10 - protein, 275 aa, Unknown
  Viruses
MSVVINNVNAVIKSLVNKKMMNEWTVLRRGEPDKFFHRFNPTLDLNVIDRDVHAEILDKFKVDIGFGLEKHLQRTNGSGMSLSNRIMKALNKIGALSRINASEILRNYNKGYDLYGRLMPKLSFDQMIADLWENQRRLLALGARLAKGLDKQMIFKTNNTEDLKCFKFSTRGDDYYIRARSTDYVNMGHHLCLAFEVLKEAGTLEYSSGAKCPIGSNCILIYRPNESSSTKLPTKPVPVRSNEKHSEQIDYFNKQIEELIFLFNNMMMKFSDYLD

SEQ ID NO: 11 - protein, 133 aa, Unknown
  Viruses
MKQLIIKRLNLLICCLCIVIAYGYYAINDYMHYKDYDVTVVNTLTGTQGKGSSLSFIAVYELKDGYRFSEYISPETYSSIEKGDNITVSLRPFDVKQTWFDNIVWFFGMALVQSICGTYIVCSILFRVIGKIE

SEQ ID NO: 12 - protein, 175 aa, Unknown
  Viruses
MNAKDIFNLVNYNDGKFKSEAQSKFFNDISIGGEITVNGGQIYKSRWNWIVIIDEIGIVEIYKNTNKNRTLHWSRDTNEQYKKDKASKLSRVTQEDIEFIKKDILMYDNLIAEEQAVIDKFDEIKASREIPDFMKESVNERYTLISERIETYKKQRAERQNTLRKFEERLNTVLA

SEQ ID NO: 13 - protein, 100 aa, Unknown
  Viruses
MLYSKAREIYETKIKEAVFQFATTMRWTNDWEYSKNHKKPLVTRKAHMLVLIDREQIKAREALQNHKKAAFEWFMDNTAPETKKAVSAWFSGKNCERSFF

SEQ ID NO: 14 - protein, 325 aa, Unknown
  Viruses
MKTVTINKGMYFGKEISGTFELLGEWFPDNAPVDAQGDGKVFVEIDGKRRGVWVYKSDISYDGVKVEEVKESYEDMKTRINKRFNVMGMMTNGIINGNIRSLIISGAAGIGKTYSLDKALNKANDIGYIEYKSINGKISGIGLYEQLWNNREENSVLLIDDVDVFSDMDILNLLKAALDTGETRKVCWSTASSYLEEKGIERELEFKGTIVFITNVDIDRELDRGTKLAPHLQALVSRSVYLDLGVHTNEEIMVRVEDVILSTDMMQKRGLSDEETYKALSWMKVNVNRLRNVSLRTALYLADFIMTDKNGWEEIATGYSSEINS

SEQ ID NO: 15 - protein, 336 aa, Unknown
  Viruses
MKTVVKSYFGSHLYGTSTPESDVDFKEIFVPPARDILIGNVKEHMSKNTNNTSSKNTKDNIDHELYSLKYFFKLAADGEPVALDMLHTPPELVVKSDLPDVWKFIQDNRSRFYTTNMKSYLGYVRKQASKYGVKGSRLAALRDVLKVVNQIPEQWVDYQEDGSIKQRRTKVEDIKHRLPENEFCEWVFHNHEKTGPQTFYTVLGRKYQTTLSLIELKQSLNKLDAEYGERARKAEANEGIDWKALSHACRGGLQLLEIYKTGDLVYPLQDAPFILDVKLGKHPFKTVQEFLEDVVDQVEAASTEASKNGMQQKVDMSFWDDFLERVYLENHRSYYK

SEQ ID NO: 16 - protein, 229 aa, Unknown
  Viruses
MNYTKVYNNLIKKGDNLVLLTAREHFIAHWLLAKIHYNSPGLIYAWWSFYNFGEDSLGRNLKLTSRGYQLVREKFSKIHSNTMKEMWKSNEYREKRSITLSLPEIRAKISESQLEAQNKPEVKEKISKGVKAAFKRPGVKEKHSAAVKKSLNNFEAKKKQSNSSKIRQRTGKHWQDYDLLYKLWIKLNRPKRGSFGTYISKLGYPKSNYHRLIVQFNEDYERSNNENCS

SEQ ID NO: 17 - protein, 181 aa, Unknown
  Viruses
MMSEAKRLVLEVSPLFGELAIEKVNNMYRLTQEDDMLYFTPSEIVRLTQIEYAYTDKIVSINDEHKIHFYSSCPGFNIKSESMCLSINNWDNFITNIKYFYDSTKRKHNLKWFKKCNAIITNSCNQNDETILNVSKCYEEGDVVSIRQIDDFRSHIITLKKEEAIALKTYLDSVIPTMISK

SEQ ID NO: 18 - protein, 34 aa, Unknown
  Viruses
MKVLFVIYVMIQYNYPMFTYNLVNNIINMIQRSM

SEQ ID NO: 19 - protein, 38 aa, Unknown
  Viruses
MKTEKQMFLMKLIEEYANAVSDYECSSRERGTAFPRKK

SEQ ID NO: 20 - protein, 64 aa, Unknown
  Viruses
MLTREQFEKIIKLAHDIEIDSYQLAVEHCEGYSYDGIEAAKRDLDKSKAKLVQYLEMIRWNNEN

SEQ ID NO: 21 - protein, 60 aa, Unknown
  Viruses
MISIEQADKIKELVALIRKADEERINFALSGIEEFEAKVNNAVEALDMFLDEIIDHNTRV

SEQ ID NO: 22 - protein, 62 aa, Unknown
  Viruses
MKIEALNQEGNIYVIINGDFFVDMDEVTSEELVELLKKRYNMCDEVATHMACAIFSLSYVVE

SEQ ID NO: 23 - protein, 128 aa, Unknown
  Viruses
MKFSDFSQSGKPSKADEYLGLLMAAQAYFHSAHFETKSYARHKAYDFIFSELPDLIDKFGEQYLGYSGRKYTPSIPDASKLPTDTIKMIDRILDQSNSIYKEMPPAIQSTIDDITGMFYQSKYLLSLE

SEQ ID NO: 24 - protein, 97 aa, Unknown
  Viruses
MALKATALFAMLGLSFVLSPSIEANVDPHFDKFMESGIRHVYMLFENKSVESSEQFYSFMRTTYKNDPCSSDFECIERGAEMAQSYARIMNIKLETE

SEQ ID NO: 25 - protein, 70 aa, Unknown
  Viruses
MLQLTEKQLRNLTVLQLDEIRREVGNIISALRREVSLNQSPADYTRLRNFEKYLDKVKAVHRHKVNTGQK

SEQ ID NO: 26 - protein, 193 aa, Unknown
  Viruses
MASLIFTYAAMNAGKSASLLIAAHNYKERGMSVLVLKPAIDTRDSVCEVVSRIGIKQEANIITDDMDIFEFYKWAEAQKDIHCVFVDEAQFLKTEQVHQLSRIVDTYNVPVMAYGLRTDFAGKLFEGSKELLAIADKLIELKAVCHCGKKAIMTARLMEDGTPVKEGNQICIGDEIYVSLCRKHWNELTKKLG

SEQ ID NO: 27 - protein, 62 aa, Unknown
  Viruses
MITREQKNEILFLVGEIISLEKDLSFEISSEYGDAETYYELVKSIDKAENDLETYLENLTKD

SEQ ID NO: 28 - protein, 61 aa, Unknown
  Viruses
MSLSKEQKDTLFSLIHEVMDKNSELEKVCNECGPFSANEYEELSKEFDNKEQELIDYINSL

SEQ ID NO: 29 - protein, 70 aa, Unknown
  Viruses
MNIHYPHPYDPKNKAVIIRQWERICRTKCPINSPHDVDKDYIGTFVEYTFIDKKGRKQHVEEYCLKVTWL

SEQ ID NO: 30 - protein, 155 aa, Unknown
  Viruses
MIVKYIKGDIVALFAEGKNIAHGCNCFHTMGSGVAGQLTKAFPKILEADKLQTEWGDVTKLGSYSVYEKYFRTHKAYCFNLYTQFQPGPNFEYSALMNCMLELNEFGENKLIKPTIYMPRIGAGIGKGNWDIIEGILDTYSSKLEIVIVDWEPLL

SEQ ID NO: 31 - protein, 115 aa, Unknown
  Viruses
MTKILVLCIGLISFSASASADTSYTEIREYVNRTAADYCGKNKACQAEFAQKLIYAYKDGERDKSSRYKNDTLLKRYAKKWNTLECSVAEEKDKAACHSMVDRLVDSYNRGLSTR

SEQ ID NO: 32 - protein, 181 aa, Unknown
  Viruses
MRKALLAGLLAISMMAHSSEHTFSNVQLDNMRYAYQFGEQFSKDGKYKTHKNIHKSGLGHIMAAILWQESSGGVNLKSKPKHHAYGMFQNYLPTMRARVKELGYNMTDAEIKRMLNKRSNSASWAYIELSYWLNIHKGDIRKAISSYNSGWNVKAGSKYASEVLEKANYLKNNKLLEIVND

SEQ ID NO: 33 - protein, 153 aa, Unknown
  Viruses
MTINTEVFIRRNKLRRHFESEFRQINNEIREASKAAGVSSFHLKYSQHLLDRAIQREIDETYVFELFHKIKDHVLEVNEFLSMPPRPDIDEDFIDGVEYRPGRLEITDGNLWLGFTVCKPNEKFKDPSLQCRMAIINSRRLPGKASKAVIKTQ

SEQ ID NO: 34 - protein, 92 aa, Unknown
  Viruses
MAQLSAGFGYEYYTAPRRVSVAPKKIQSLDDFQEVVRNAFQDYARYLKEDSQDCLEEDEIAYYTQRLEQLKNLHEVRAEVSKSMNKLIRFKE

SEQ ID NO: 35 - protein, 88 aa, Unknown
  Viruses
MIEDIKGYKPHTEEKIGKVNAIKDAEVRLGLIFDALYDEFWEALDNCEDCEFAKNYAESLDQLTIAKTKLKEASMWACRAVFQPEEKY

SEQ ID NO: 36 - protein, 58 aa, Unknown
  Viruses
MAKIIIEGSEDVLNAFASGLVTQANSNLMKRGIWVILMEFILRQKFLFKAMAFMNLFV

SEQ ID NO: 37 - protein, 120 aa, Unknown
  Viruses
MKAYQILEGTHKGTIYFEDGIQARIIVSKTFKEDSFVDPEIFYGLHAREIEIEPQPTVKIEGGQHLNVNVLRHETLEDAVKHPEKYPQLTIRVSGYAVRFNSLTPEQQRDVIARTFTESL

SEQ ID NO: 38 - protein, 109 aa, Unknown
  Viruses
MMTDTQLFEYLYFSPKTIKNKLVNHFEILAKNNILSEFYPKQYKLQKGVFKGCRVLCTAPNARLMNKIPYFTMEFIDGPFKGLITQSLMAYDSEPFLIKEQSWINLFSN

SEQ ID NO: 39 - protein, 219 aa, Unknown
  Viruses
MRDSRQPVIRSSPSAVMGKYRNGQFMCHGMAQTYRAYREEMRTFLTGPYLSLMNAFTHHSDARVEEICKNEYIPPFEDLLKQYCTLRLDGGRQSGKSIAVTNFAANWLYDGGTVIVLSNTSAYAKISANNIKKEFSRYSNDDIRFRLFTDSVRSFIGNKGSKFRGLKLSRILYIIDEPVKSPDMDKIYSVHIDTVHYCCNSKCCIGGITRPQFFVIGMQ

SEQ ID NO: 40 - protein, 138 aa, Unknown
  Viruses
MTRINLTLVSELADQHLMAEYRELPRVFGAVRKHVANGKRVRDFKISPTFILGAGHVTFFYDKLEFLRKRQIELIAECLKRGFNIKDTTVQDISDIPQEFRGDYIPHEASIAISQARLDEKIAQRPTWYKYYGKAIYA

SEQ ID NO: 41 - protein, 193 aa, Unknown
  Viruses
MKTYQEFIAEASVVKAKGINKDEWTYRSGNGFDPKTAPIERYLATKASDFKAFAWEGLRWRTDLNIEVDGLKFAHIEDVVASNLDSEFVKADADLRRWNLKLFSKQKGPKFVPKAGKWVIDNKLAKAVNFAGLEFAKHKSSWKGLDAMAFRKEFADVMTKGGFKAEIDTSKGKFKDANIQYAYAVANAARGNS

SEQ ID NO: 42 - protein, 100 aa, Unknown
  Viruses
MKTYQEFIAEARVGAGKLEAAVNKKAHSFHDLPDKDRKKLVSLYIDRERILALPGANEGKQAKPLNAVEKKIDNFASKFGMSMDDLQQAAIEAAKAIKDK

SEQ ID NO: 43 - protein, 157 aa, Unknown
  Viruses
MKTYQEFITEAAINSQIIAESFTDLLKFKKGQKITAVLDDGTEVEMDVQGYNYAVDGKLYNKSHAKFDSFDDFVNTVEDEKTRRSIATGDAKVLMAHGHERIRAKQNKMGDENFALVGYQSGKQTYGYQRTATMYNKNGKIAFVNSKGSIQYVKSFK

SEQ ID NO: 44 - protein, 71 aa, Unknown
  Viruses
MKTRSQIEDMVRNASYTRDAMTFLCENNLDPDRVNSSIHFKYMNSSEWLRHFDKAGYITQMTAREQLTHSN

SEQ ID NO: 45 - protein, 102 aa, Unknown
  Viruses
MMTTIEVFEYCYNSPVCNKRALVENYEIYHFKPKRYRLTKGPFAGQQVLCTAPNARLMTSIPHFKMEFIDGPFKGLITQSLMAFNSDPFLIKEKTWINLFSN

SEQ ID NO: 46 - protein, 99 aa, Unknown
  Viruses
MAQILSGFGTHYEASRRITESNPFGLVPKHKKIQSLDDFENRLWALFNEYKAYLKEDADDCLEEDEIAYYEQRLEQLKNFHQVRDEVSKAVKKLIPFKE

SEQ ID NO: 47 - protein, 115 aa, Unknown
  Viruses
MTKMLALIVGLVSFNALANTTYTDVTEYTNRTASDYCGKSQECKVDFSQKLLYAYKDGEKDGASSRFKASTLIKRYYKKWQILECSVAEPKDKAACNSMVDRLVDSYNRGLAASD

SEQ ID NO: 48 - protein, 70 aa, Unknown
  Viruses
MQQLNERQLRNLTVTQLDEIRRELGHSISHLNEDIRQTGSKADYTRKRKLEKYLADVKAVQRRKINTGQN

SEQ ID NO: 49 - protein, 100 aa, Unknown
  Viruses
MALRAIAVMAMLGFFAATTPIVGTAYVDPYFDNFMESGIKNVYTLFEIQNVENSEKFYKYMAKHYKNSPCDDAFECHEQGIKTARQFAEFMKIKLEPTSI

SEQ ID NO: 50 - protein, 59 aa, Unknown
  Viruses
MTRKEKISKLMFLIEEYANSVSDWENAHGCEDGDIDINRAMKKMADAHTELQMYVNEIM

SEQ ID NO: 51 - protein, 55 aa, Unknown
  Viruses
MLYDYTGKSEDGVLELLPESAEDDDMVVIYCVGCQSMHDEIFKREPRNCWHRSMR

SEQ ID NO: 52 - protein, 37 aa, Unknown
  Viruses
MAQEHEMIYYLEPWAWLTMAVGPVLIGLFFSWLARKL

SEQ ID NO: 53 - protein, 71 aa, Unknown
  Viruses
MLYDPSGNSENGVIVLEPEHPVGDVYRPVEVCECNFVKGNTGGISIEQDDDVIYLDASQVEALYSILKHNR

SEQ ID NO: 54 - protein, 36 aa, Unknown
  Viruses
MKLLLIAYVVVQYNYPMFTYNMVNGIVNLIETSMVK

SEQ ID NO: 55 - protein, 59 aa, Unknown
  Viruses
MTREQANKLMDLIHDLREADSDLNDVAYHAVNDNGDFYENQVDACQNALVNFVETLIGE

SEQ ID NO: 56 - protein, 176 aa, Unknown
  Viruses
MKTVIETTELFGDLCIEKRGYAYVLTQEDDAVTILPMELDKILKLNPPGHASVINIDEDLQVRFYHGLYSGVNIETEDECFSINNWKTFVTKVKEFMESETVKKAKLQWAKCRNAFITNQDRPDYTTVLGVNPSYEDGDVVVIRQIDDLRQHIITLDKDEAVALKAYLDSIIPTLK

SEQ ID NO: 57 - protein, 157 aa, Unknown
  Viruses
MIINENSWHFKIYAAFNSTWNRPKTLCAYFWKTFLPVLFVSIIGFAILAAATIVGQEILTKFFVFSSLWTLVPASIGVGVLFIALCVGISIVLVLGIPWLIDLYRERKYYKEVELIAQNQERIKNGLEPIERKKSLIGEYLKARKEKVCPTLEYKAK

SEQ ID NO: 58 - protein, 95 aa, Unknown
  Viruses
MDFMQKIKAAVELYAHRMVSSTMQYSEEVRQDSSLKGKTKALITLDRLQKNALELFQYDKDAYKKEQEEKLKSEKLQKVPKSVWFSGKNTEKSFF

SEQ ID NO: 59 - protein, 331 aa, Unknown
  Viruses
MSITSDAVKHEINGLCASQIKVWFKDHNDRRALNYEQIAIQVHQLLTSKFDFHMPSEIYTNLPRALIKACKGYGKSMADGIFSALNKLNVLSMINSNNILRNLGRTDLFGREQCKMPLKWIVAHLNENQTRFMAVSARLQMGLDRQMNFKTHPRYSGSTDFYKCELQKVAGKTVLVVRVTFEGRRQGYFDLIGSGLKHCGFIDEVDIKRDSIGYRIGYVCTLKEEISGPAPGLKDVRYKDELVEEIKEVEYDINQEDALKDLIDELNQDVIPVQKFESKHAEQIKYFEALINELNVTIQQTDDEISRLAGLNGKNKTERANLIQVVKLLKE

SEQ ID NO: 60 - protein, 311 aa, Unknown
  Viruses
MASITRRELIKEFTSNAYTPGPNPIRMPVKTPEEIEEICSYYGITSRKFTQFECVTNTPVKEFIKNGEFSRLLAKQKLRNFCIGGEGQYFAKQFRNNLNNLMAIASRIRPQAINMKHLKYTYDKLEIMVISENEFTLTYKPKNDNVARVFHQALEVLGDNIHQIKAIYSRELNIINPIKNKGHIAIQARVSIGKKVPAPWWHKDSEYYQNLKKENATFEASITTTPIGNAEVQGAYFTAPSIYTSPSTKLEISNGLLPAEDYHYNQIKATMDKLSVELDKAQAKLRDAQSVVTNLQLQYSKLFNAMNALKS

SEQ ID NO: 61 - protein, 322 aa, Unknown
  Viruses
MKTVTINKGIYFGKEISGTFELLGEWFPDNAPVDAQGDGKVFVEIDGKRRGVWVYKSDISYDGVKVEEVKESYEDMKTRINKRFNVMGMMTNGIINGNIRSLIISGAAGIGKTYSLDKALNKANDIGYIEYKSINGKISGIGLYEQLWNNREENSVLLIDDVDVFSDMDILNLLKAALDTGETRKVCWSTASSYLEEKGIEREFEFKGTIVFITNVDIDRELDRGTKLAPHLQALVSRSVYLDLGVHTNEEIMVRVEDVILSTDMMQKRGLSDEETYKALSWMKVNVNRLRNVSLRTALYLADFIMTDKNGWQEIAEVTLLK

SEQ ID NO: 62 - protein, 244 aa, Unknown
  Viruses
MKTVMKSYFGSHLYGTSTPESDVDFKEIFVPPARDILIGNVKEHMSKNTNNTSSKNTKDDIDHELYSLKYFFKLAADGETVALDMLHTPPELVVKSDLPDVWKFIQDNRSRFYTTNMKSYLGYVRKQASKYGVKGSRLAALRDILKVVNQIPEQWVDYQEDGSIKQRRTKVEDIKHRLPENEFCEWVFHNHEKTGPQTFYTVLGRKYQTTLSLIELKQSLNKLDAEYGDVLVRLKRMRALTGKL

SEQ ID NO: 63 - protein, 161 aa, Unknown
  Viruses
MHILMQCLGNKISDFNHKRVNDYIKDKLEIKDILFRGLTIEEVLSYKFEVNRRIKFKRITSFSSESHIAETFAAERYMTNVLVVLKNANVFDYSTAMIDILENLIAIEESGQTDDDKLNKLYDNLCMVDYEREFLLPINSELIVKNIYFDSKKNMHIVEME

SEQ ID NO: 64 - protein, 180 aa, Unknown
  Viruses
MKRLVLEVSSLFGELAIEKVNNMYRLTQEDDMLYFTPSEIIHLTQIEKPYTDKIVSINDEHKIHFYSLCPGFNIESESICLSINNWDNFITYIKYFYYSNERKHSLKWLKNCNAIITNACDQCDQNDETVLNVSKCYEEGDVLTIRQIDDSRAHIVTFTKDEAIALKTYLDSVIPTMISK

SEQ ID NO: 65 - protein, 165 aa, Unknown
  Viruses
MKKYFTIYKTEILNLKTGIKKYYYGRHITNNINDSYVGSGNYIERVKVSNDHILSKTVLEVFDNYDKMVQAEILYIMAGKLKYGKNCVNLSYGGEGGKVADETRIKCKKHSINLWKNPNHIQHMKKKMKEVTSTPEAKLNTSIKTSQRYKDPVLLEKLVILLKRN

SEQ ID NO: 66 - protein, 93 aa, Unknown
  Viruses
MKTYNEFLSESILNEATDTFATKLGAALVEAESLLARISELASKIDTRKFERTSDIVKLEALLRMCDKPEEIAKNGSLMKQRLEKYISGASEK

SEQ ID NO: 67 - protein, 239 aa, Unknown
  Viruses
MKKYFTIYKTEILNLKTGVKKYYYGRHITNNINDSYVGSGNYIERVKVSNDHILSKTVLEVFDNYDKMVQAEILYIMAGKLKYGKNCVNLSYGGEGGKVADETRIKCKKHSINLWKNPNHIQHMKKKMKEVTSTPEAKLNTSIKTSQRYKDPVFVRKVSNIIKKKLSNPAVKEKHINGIKAAKRSHLTIWQYEDELFELWKETKCGWRKFTKEAIKHGYPNEPYRSMVSTFQKRHLEES

SEQ ID NO: 68 - protein, 73 aa, Unknown
  Viruses
MKTVMKSYFGSHLYGTSTPESDVDFKEIFVPPARDILIGNVKEHMSKNTNNTSSKTLKMILTMNYTVLNISLN

SEQ ID NO: 69 - protein, 152 aa, Unknown
  Viruses
MKTVTINKGIYFGKEISGTFELLGEWFPDNAPVDAQGDGKVFVEIDGKRRGVWVYKSDISYDGVKVEEVKESYEDMKTRINKRFNVMGMMTNGIINGNIRSLIISGAAGVGKKDSFDKTFNKTKDIWKIEYKNINGKISGIGLFEQLWNCLF

SEQ ID NO: 70 - protein, 155 aa, Unknown
  Viruses
MDILNLLKAALDTGETRKVCWSTASSYLEEKGIDREFEFKGTIVFITNVDIDRELDRGTKLAPHLQALVSRSVYLDLGVHTNEEIMVRVEDVILSTDMMQKRGLSDEETYKALSWMKVNVNRLRNVSLRTALYLADFIMTDKNGWEEIAETVLLK

SEQ ID NO: 71 - protein, 64 aa, Unknown
  Viruses
MSLSKEQKDRLFSLIHEVMDKNNELEKVCDESDYGDAETYYELVKSIDKAENDLETYLENLTKD

SEQ ID NO: 72 - protein, 57 aa, Unknown
  Viruses
MNNFELRYEVLRNIDNLIELAVNKGFAIGIGQKDTTFIDKNKRKQHVEEYCLKVTWL

SEQ ID NO: 73 - protein, 70 aa, Unknown
  Viruses
MKVHYPHPFDPKNKAEIIRHWTETRVTKCPIKSQHNTDKWYIGEYVEYTFIDKNKRKQHVEEYCLKVTWL

SEQ ID NO: 74 - protein, 50 aa, Unknown
  Viruses
MGTFLGGDGKEINTYAKRSEIAYVVASTKVDVDDIGYDFNNFGIISGGKA

SEQ ID NO: 75 - protein, 179 aa, Unknown
  Viruses
MRTFLTGPYLSLMNAFTHHSDARVEEICKNEYIPPFEDLLKQYCTLRLDGGRQSGKSTAVTNFAANWLYDGGTVIVLSNTSAYAKISANNIKKEFSRYSNDDIRFRLFTDSVRSFIGNKGSKFRGLSLSRILYIIDEPVKSPDMDKIYSVHIDTVHYCCNSKCCIGGITRPQFFVIGMQ

SEQ ID NO: 76 - protein, 42 aa, Unknown
  Viruses
MYQEFIVEADAKGPKFKVKAFIGDAEDFGSLKRNGYFLRWRW

SEQ ID NO: 77 - protein, 98 aa, Unknown
  Viruses
MKTRSQIEDMVRNASYTRDVMTFLCENNLDPDKVNRVIYHFKYTNSSEWLRHFSKAGYITQMTAREQLTDFCKTIDYKNPLFVRGVGQSKTDLSTGFF

SEQ ID NO: 78 - protein, 340 aa, Unknown
  Viruses
MKTRSQIEDMVRNASYTRDVMTFLCQNNLDPDKVNRVIHRFKYTNSSEWLRHFSKAGYITQMTAREQLTDFCKTIDYKNPLFVRGVGQSKVDLSSGFFNPNHYRIEWRFIALFRKQLKQILSIASRLKGSDINLKNLKFDGYTLQMEVRPLKENNRTARISFKPNTKNSLSICECLKSQLTEAFKYMDVVAAVQSKILPHFERNWEHTTTYELDMIVSFKYEFLRKDEIVQEKKQEVQNTLNSSNYSSNDPKFWMYSSSNIDACKLNKVSFLPTENSNFKPVEKWHADAIEKSLKAVDDELVKATNEVLEAEKVLKQAQSRVQNLTKQRSKLNNALNALN

SEQ ID NO: 79 - protein, 308 aa, Unknown
  Viruses
MTRKHYIDYFDSLITKHRNYQIGRRAVINNILRDFLQYVGQENHICKDTQNAYSHSLGNLLQWFKRSRLLSSTVASDNIKNFMKPSFIKSETSITDLVEFTIVNDVKKTHLADWLSTIPETKFADKFACQFNDQVNMLFKHARKLFTAGDDRTNTVHVKDWVIADEVTRKPGGSSVLINIQVPYYYSHNLGTMTAREINKHNKIIRSLSYKLCTMVEMMDVVEMYDETEDNGSMLYSSRILIKLKNPNTYKQVVKEPKAEKADNLSEEREYLNARLIEVEAQIAEHTKLLKALNAKANGLRNAIEVLK

SEQ ID NO: 80 - protein, 296 aa, Unknown
  Viruses
MSVVINNVNAVIKSLVNKKMMNEWTVLRRGEPDKFFHRFNPTLDLNVIDRDVHAKILDKFKVDIGFGLEKHLQRTNGSGMSLSNRIMKALNKIGALSRINASEILRNYNKGYDLYGRLMPKLSFDQMIADLWENQRRLLALGARLAKGLDKQMIFKTNNTEDLKCFKFSIRGDDYYIRARSTDYVNMGHHLCLAFEVLKEAGTLEYVSGAKCPIGSSCILIYRPDESSSTKLPTKPVPVRSNEKHSEQIAYFNKQIEELNISIQQYDDEIFRLSGLSSKAKSEREKLIKIVDLLKS

SEQ ID NO: 81 - protein, 120 aa, Unknown
  Viruses
MAAILWQESSAGVNLKSKPKHHAYGMFQNYLPTMRARVKELGYNMTDAEIKRMLNKRSNSASWAYIELSYWLNIHKGDIRKAISSYNSGWNVKAGSKYASEVLEKANYLKNNKLLEIVND

SEQ ID NO: 82 - protein, 409 aa, Unknown
  Viruses
MSVVINNVNAVIKSLVNKKLNEWTVLRRGEPDKFFHRFNPTLDLNVIDRDVHAEILDKFKVDIGFGLDKHLQRTNGSGMGLSNRIMKALNKIGALSRINASEILRNYNKGYDLYGRLMPKLSFDQMIADLWENQRRLLALGARLAKGLDKQMIFKTNNTEDLKCFKFSIRGDDYYIRARSTDYVNMGHHLCLAFEVLKEAGTLEYSSGAKCPIGSNCILIYRPDESSLTKFPTKPVPVRSNEKHSEQIDYFNKQIEELNISIDKQMIFKTNNTEDLKCFKFSIRGDDYYIRARSTDYVNMGHHLCLAFEVLKEAGTLEYSSGAKCPIGSNCILIYRPDESSLTKFPTKPVPVRSNEKHSEQIDYFNKQIEELNISIQQYDDEIFRLSGLSSKAKSEREKLIKIVDLLKS

SEQ ID NO: 83 - protein, 73 aa, Unknown
  Viruses
MAKIIIEGSKDVLNAFAEWFSNSGEQQFNEAWTMGDIDGIYPTTEISVQGYGIHEPIRLVEYDLCTGEEVKYD

SEQ ID NO: 84 - protein, 129 aa, Unknown
  Viruses
MMLEGTDYIHDYRGSAVYVGDEVAVYYGYGTLMTAKVIQIKNNRAKLEVYYSNGEKSISKWKYGDCMVKLGVNMIYDISVSRTPSMVTIPAEELDRLHKIEELLWEIESDLPSGLESWIDYEELNKLRN

SEQ ID NO: 85 - protein, 66 aa, Unknown
  Viruses
MTTLNKRFGKAKSGCAAERRYVFAQLEDVEYQINQVHITGIEPMCGLEALREVRDGYRRDIEEMSK

SEQ ID NO: 86 - protein, 70 aa, Unknown
  Viruses
MVMSQTSILKNAHCEKCKWPVVFALCNDEMACDFDYWCYCSNKGCINHKGEGFYSGFYPYPDFVKEGKPK

SEQ ID NO: 87 - protein, 58 aa, Unknown
  Viruses
MNDDLKYQLLRELDVLIELSAQKGFIIGSGQKDPNGHSIVAVMNQKRVILKLLGIDIL

SEQ ID NO: 88 - protein, 159 aa, Unknown
  Viruses
MEKTVLTCHTGGGGEGGKVADETRIKCKKHSINLWKNPNHIQHMKKKMKEVTSTPEAKLNTSIKTSQRYKDPVFVRKVSNIIKKKLSNPVVKEKHINGIKAAKRSHLTIWQYEDELFELWKKTKYGWRKFTKEAIKHGYPNEPYRSMVSTFQKRHLEES

SEQ ID NO: 89 - protein, 96 aa, Unknown
  Viruses
MKKYFTIYKTEILNLKTGIKKYYYGRHITNNINDSYVGSGNYIERVKVSNDHILSKTVLEVFDNYDKMVQAEILYIMAGKLKYGKNCVNLSYGGGG

SEQ ID NO: 90 - protein, 61 aa, Unknown
  Viruses
MTSEQAFKLRELIETYSKAVHTATVIDESAFSGHANKIKYKTLMEEAKVNLDSYIETLIGE

SEQ ID NO: 91 - protein, 87 aa, Unknown
  Viruses
MTVYVDVLMNHGWKLRGHPTKNCHMFTDGDIEELHEMAEAIGMKRSWFQDKRIKHYDLHARRRQKAVELGAVEVSRREAVKIWRTLK

SEQ ID NO: 92 - protein, 49 aa, Unknown
  Viruses
MKITPIEVKKLIDTEEISECFESFLEDATEDNAVYLAQKIIETYLEKNQ

SEQ ID NO: 93 - protein, 173 aa, Unknown
  Viruses
MFISSGSGLIRVEFKNDIFLIQGDDIIKMSYDEIKKICHALESHGKVNAVIDIGDLWVTLYEVSEGFNIEDENNILAIDKRSDLFDVLKVYEQSNGGRKAVLIYQKPHSCGTASIISDIEDETDTYMCVLKAGGDRHPDFISIRQNNEEISLSKSEAEAMIKYLTTVTPSMKG

SEQ ID NO: 94 - protein, 98 aa, Unknown
  Viruses
MKKRLLEDIAEASDFPEWTPCAGFDKGLLVTDDLDFKPPAWDAIMAMVERRERASKNVPNCPECGTEQVQLVHWQTSNLRYKCRHCKHRFNREENDKA

SEQ ID NO: 95 - protein, 67 aa, Unknown
  Viruses
MTIENKLDVDAVLSEIIEDHDAFSENYDFDFSDYLKPIEIEDWVQDGKCQYRQCVYFSPKHNVHGCK

SEQ ID NO: 96 - protein, 155 aa, Unknown
  Viruses
MFGQNSVLSIINQWLNSVDAGIVSSAKFIHEIRKISSRVDKDVIKKTFKESRLLSYLVNRDILGNFGKEIKRTKDVVGYNWFGDVNSYHLNVKEDPENIFTRRWISNFRLFKKQILKSASKLCYGDYRQIHPLASDMIIIKEYELDKNKAAFCEL

SEQ ID NO: 97 - protein, 182 aa, Unknown
  Viruses
MMNEWTVLRRGEPDKFFHRFNPTLDLNVIDRDVHAEILDKFKVDIGFGLEKHLQRTNGSGMSLSNRIMKALNKIGALSRINASEILRNYNKGYDLYGRLMPKLSFDQMIADLWENQRRLLALGARLAKGLDKQMIFKTNNTEDLKCFKFSTRGDDYYVRARSTDYVNMGHHLCLAFEVLKEA

SEQ ID NO: 98 - protein, 99 aa, Unknown
  Viruses
MKQLIIKRLNLLICCLCIVIAYGYYAINDYMHYKDYDVTVVNTLTGTQGKGSSLSFIAVYELKDGYRFSEYISPEMYSSIEKGDNYCKFTSFRRKTDIV

SEQ ID NO: 99 - protein, 55 aa, Unknown
  Viruses
MKNLISFGVKPWWAARWETVEPEPEEPVYTDEETVYNEPTINDLIDMEMGHDYSR

SEQ ID NO: 100 - protein, 119 aa, Unknown
  Viruses
MFISSGSGLIRVEFKNDIFLIQGDDIIKMSYDEIKKICHALESHGKVNAVIDIGDLWVTLYEVSEGFNIEDENNILAIDKRTDLFDVLKAYEQSKWRKKSCIDLSKTAFMWNCFNHFKY

SEQ ID NO: 101 - protein, 105 aa, Unknown
  Viruses
MKRLVLEVSPLFGELAIEKVNNMYRLTQEDDMLYFTPSEIVRLTQIEYAYTDKIVSINDEHKIHFYSSCPGFNIKSESMCLSVIHWDSFIAKIKYFIILMKENIV

SEQ ID NO: 102 - protein, 57 aa, Unknown
  Viruses
MKTEKQMFLMKLIEEYANAVSDYECSSRERGTAFAKEEMKIMVDAHTKLQNFIENVI

SEQ ID NO: 103 - protein, 153 aa, Unknown
  Viruses
MIINESSWHYKLFKMFNDEWKRPKTLCAYFWSIVIPTFFVSFFGCTILAGLTIICAEIMQKWLIFGSLWTLIPSAFILAILLVLLIIGSFVIPAQLHEKYKDYKWKKDYALHVENIDRAYKGLPPIQPKKSIIVEFLKARKAKVCPVIEYKAE

SEQ ID NO: 104 - protein, 180 aa, Unknown
  Viruses
MKAVIETTDLFGDLVIEKRGNVYVLTQEDDSITILPMELAAILSYAPSGVTGVTNITGDLQVRFYRGLYGGNIETEFMCLSINNWATFVAKVKEFLESETAETPEKAKLQWAKDRGANIINPVAANYTTTLDVKTSYEDGDVVLVRQNDDSRAHIVTFTKDEAIALKTYLDSVIPTMISK

SEQ ID NO: 105 - protein, 57 aa, Unknown
  Viruses
MTKRKEYMETAEKAVRELAIAYYNEHGKFPDRYSVLKSALTRSYKNMLSEVSDIIQT

SEQ ID NO: 106 - protein, 68 aa, Unknown
  Viruses
MVAMISPPVNWDSVMVMVERRERASKNVPNCPECGTEQVQLVHWQTSNLRYKCRHCKHRFDREENDKA

SEQ ID NO: 107 - protein, 57 aa, Unknown
  Viruses
MFISSGSGLIRVEFKNDIFLSQGDDIIKMSYDEIKKICHTLESRGKVNAVLTLVIYG

SEQ ID NO: 108 - protein, 97 aa, Unknown
  Viruses
MICYITPSEIVRLTQIEYAYTDKIVSINDEHKIHFYSSCPGFNIKSESMCLSINNWDNFITNIKYFYDSTKRKHNLKWFKNVMLLLLTPVIRMMKLF

SEQ ID NO: 109 - protein, 88 aa, Unknown
  Viruses
MGFPKLEVGDLVLTKLWNDAQSVEICQYRGATGNLMYTIYNPEILLECHLERFIKDTDSMPYSVSIVRKSDTKEYSKILEQIRSNKKD

SEQ ID NO: 110 - protein, 44 aa, Unknown
  Viruses
MSESKRINMKRLVLEDSVLFGELAIEKVNNMYRLTQEDDMLYYA

SEQ ID NO: 111 - protein, 195 aa, Unknown
  Viruses
MTRINLTLVSELTDQHLMAEYRELPRVFGIVRKHVANGKRVKDFKISSEFILGSGHVTFFYDKLEFLRKRQSDIITECLKRGFSIKDTEVPDISDIPTRGKILMAQLSAGFGYEYYTAPRRVSAAPKKIQSLDDFQEVVRKAFQDYARYLKEDSQDCLEEDEIAYYEQRLEQLKNLHEVRAEVSKSMNKLIRFKE

SEQ ID NO: 112 - protein, 58 aa, Unknown
  Viruses
MYQEFIVEADAKGSKFKVKAFIGDVEDFGSLKRNGYSFLGGDGKEINTYANVVKLLML

SEQ ID NO: 113 - protein, 84 aa, Unknown
  Viruses
MYQEFIVEADAKGSKFKVKAFIGDAEDFGSLKRNGYSFLGGDGKEINTCAKRSEIAYVVASPKVDVDDIGYDFNNFGIISGGKA

SEQ ID NO: 114 - protein, 71 aa, Unknown
  Viruses
MMLEGTDYIHDYRGSAVYVGDEVAVYYGYGTLMTAKVIQIKNNRAKLEVYYSNGEKSISKWKYGDCMVKLG

SEQ ID NO: 115 - protein, 157 aa, Unknown
  Viruses
MKTYQEFITEAAINSQIIAESFTDLLKFKKGQKITAVLDDGTEVEMDVQGYNYAVDGKLYNKSHAKFDSFDDFVNTVEDEKTRRSIVTGDAKVLMAHGHERIRAKQNKMGEDNFALVGYQSGKQTYGYQRTATMYNKNGKIAFVNSKGSIQYVKSFK

SEQ ID NO: 116 - protein, 39 aa, Unknown
  Viruses
MKTYQEFITEAAINSQIIAESFTDLLKFKKRTENYCCIG

SEQ ID NO: 117 - protein, 158 aa, Unknown
  Viruses
MDFFTPEANQKNINKFFSIASTITRQLETALLCMETVENIHTYPFKNICGWEGYKIVISLREVKCAYSPTDKEIYQQKCDEIVNTPKEETTLEELMECLDDSPEPVEIRPEVIALEKAYKEVLEISNKAQKEYEQAKKIWEESVNRLDRLEQALQLIK

SEQ ID NO: 118 - protein, 159 aa, Unknown
  Viruses
MIVKYIKGDIVALFLQGNIIAHGCNCFHTMGSGVAGQLARAYPKILEIDKTTTEYGSRDKLGDMSIVFKHKPNGFGICYNLYTQYEPGPNLDYGALVNCMIELNLQAETLLFKPVIYIPRIGCGIAGGDWDKVSKLIDMFTPDIDLIVVDYESTLPTSV

SEQ ID NO: 119 - protein, 61 aa, Unknown
  Viruses
MSLSKEQKDKLFELIHELLDEHTEANTFYDEYGPLSPDQQEEFADRFDKKENELIAYVNTL

SEQ ID NO: 120 - protein, 137 aa, Unknown
  Viruses
MTRINLTLVSELADQHLMAEYRELPRVFGAVRKHVQNGKCVKDFKISPTFILGTGHVTFFYDKLEFLRLRQIELIAECLKRGFKIKDTTVQDISDIPAEFRNNYVPDEASIAISQACLDEKIAQRPTWYKYYGKSIY

SEQ ID NO: 121 - protein, 92 aa, Unknown
  Viruses
MAQLSAGFGYEYYTAPRRVSAAPKKIQSLDDFQEVIRNAFQDYARYLKEDSQDCLEEDEIAYYTQRLEQLKNLHEVRAEVSKSMNKLIRFKE

SEQ ID NO: 122 - protein, 137 aa, Unknown
  Viruses
MKQLIIKRLNLLICCLCVVVAYGYYAINDYMHYKDYDVTVVNTLTGTQGKGSSLSFIAVYELKDGYRFSEYISPEMYSSIEKGDNITVSLRPFDVKQTLFDNIVWFFGMVLVQSVCGAYICCYIIFRLAKLIETEIE

SEQ ID NO: 123 - protein, 148 aa, Unknown
  Viruses
MIINENSWHVRMHDFFYWQRPNSLCKYFWKMAWTFLVIGVICASGLLGSWVVGQAILSQFGVTGFWLLHAGGTVFGVLIFATIVVIAVGIAYIVAKISGLWERVRYDRAWKRQQDEENGIEHPKSVIVEYLKASKSKICPMIEFKDVK

SEQ ID NO: 124 - protein, 105 aa, Unknown
  Viruses
MKTYQEFIAESAAGQRVEGGYIWIEQVEPPSRTSTGKWKITVDGPGGKRRTLKEINGYYEEALSDAGTYMMQIMIKNPENRFAARVFRRIGNKLYDAYSKNFPKE

SEQ ID NO: 125 - protein, 102 aa, Unknown
  Viruses
MCLSINNWDNFITYIKYFYYSNERKHSLKWLKNCNAIITNACDQCDQNDETVLNVSKCYEEGDVVSIRQIDDFRSHIITFTKDEAIALKTYLDSVIPTMISK

SEQ ID NO: 126 - protein, 70 aa, Unknown
  Viruses
MLQLTEKQLRNLTVLQLDEIRREVGNVISALRREVSLNQSPADYTRLRNFEKYLDKVKAVHRHKVNTGQK

SEQ ID NO: 127 - protein, 56 aa, Unknown
  Viruses
MIYDISVSRTPSMVTIPAEELDRLQKIEELLWEIESDLPSGLESWIDYEELNKLRN

SEQ ID NO: 128 - protein, 93 aa, Unknown
  Viruses
MKTYSEFLSESILNEATDTFATKLGAALVEAESLLARISELASKIDTRKFERTSDIVKLEALLRMCDKPEEIAKNGSLMKQRLEKYISGASEK

SEQ ID NO: 129 - DNA, 168903 bp, Enterobacteria phage T4
aattttccttattaggccgcaagggccttcatagttttagcgatttgggaaacttcatcatcacttaaagagttgcgataaccgatgaagtcggaaacaatacggaatttcttggtaaactcagcaaccattttatcactgttttttgaagcattatttgataatacatcaaaaagattagttactgtccaaatgtcatgaccgatggtatcttttccaccattaaaatatacaccctgtaatgaactaaccatattagcgagtcgtgtatattcttcagaaacttcatctatactgaagtacttcatcataaaatctaactcaggatacttgataattttatcaatatatcgtttagctgaacttgaataacctacatacttatcataatctacatcatcaaaagcatctacatataaatcacgcaaagcttcaaaaatacattggcactgaccgagttcttttacctttttctgtaaaagcggacgaataacataaaattcattaatgccaataagattagccatacgaatcaaaatattcatagatggatgacaaagagatgtagtaccatccatagagaaaatatcagaacgatgcatatacgctacataaccagtaatttcatctgcttctgatgtgaggcgtaaataattcctcttttcccagcgcccgtctttaatttcaaacttaaacgctgtagcagctttaggacgaggagctttacttttaactacctttggaatataactttttactaaagcttcaatttctgacaaataatgaatgttaacttcatcactttcaaacatcgccataatatcaggaagcaaatcaatctgcgattctacttctggattaataaacagaagacgttcgttatgatgaatattcaaagtgttattaaattcactatcatctaacgcacgtgctaatccacggacaatattaacacgatttttaatattatcaataacgatattaatttttgttgtattaataccaaacagacgataacttgatgcaacggctgaagtttcatgactttgcttaatgcgcttcagtcgagggtcaagatttacttcatacacaactcccgcgttgcataacttactgtcaggttcaaacatgctctgcatctttttatatgacagatttttagtcgtgaatttgactgaattactaatcatataatctcgagcagaataccccatcttcatcaattcacgatatgtgtgacgaggagatgtagattctttaaatcgttttacatcttcattaaatgctttctcactgagttctttaactcgttcaataatatttttacgagtgcgatcatccagtgaaagagcctcgcgagatggagcaatatcaagtgaacccattggaaacttaatgtaattcacttcattgcgaatgcttagccagttacggtctctaataacaccatcgataggataaacaataccaccgtagatagcatataatccaccacgatcaggccagtatctttctggatttacaccgtaatagtcatcaaaatccggaaaataatcaatttcgcggtcaagaccattaatgatagccaaatctttgaacggtcgcatgatataagaaacttcataagcaaagtttctaaagtctttttcttcaactggaactacgatttcaataccagttttatcatctggacccatttcttttacgaatgtaggtttaatctgtggaccatcaccatccatgtaagctacataaccacgaatttcacctttatgatacgaagtaatactaaacgtatcagtataactaaacggagatttagaacctaaaccaaatccgccaataaagtcattagattcagctttagatgaactgaagtatgaattatacaacccaggagaattatcatcaccttgaatatcaaaatcactcatacccggaccaaaatctcgacaaacaaatcgtgggtctaaacgtccaggaacttgtatgataaatttttcaggatttccattaagtgcatgagcatcaatcatgttagtaatcaattcacggactactgcgcgaatcttgtttgtatacaaatcagatgacagaattttaaatactttaggagatgctgtgatgctaaatgcttttgatttagaaccattaccaagaattgtttctttttcagtggtgataatcataatttcctcattaattcatattacgcttaataacttcagcaacttctagtagttcatctttagttgcggtgtcggattgaattttatctctaatatctttaaagcgggttttaaattcttcggcttctcccatatcgaaaaagcgttgaatgattctatattctcgatgaactgctttatcaaaaagttctaaatttactttatatgatttcatttcaatatcctcatttgcccaattaattataccacatccttgtggtaaagtaaactactggctcatccattctttacgaaggtcagcattatctcccatgagcatttcaaaaagctctttccagttctcaggaagtttaacaacatcatatactgggttttgaatcatctcacgatattcagatttttccaaagagccaagtcccttaatataacggatgctatgtttaggtagagcatctttggcactctcatattcagcgactgtataaaaccattcttgttttttaccgacctgagcgatgattacaggagttttgacaaagcgaattcgtccttgctcaaacaattctggccaattactaaaaaatccgagcagagaaggataaatagaacctaatccaataatctcttaattatgaggtatttctatagatagcccgaaggctatccatcgtgatctgcgtctgtcataatagcgacattcgcatagttcattgaagaggatttaatagaacgacgaacattgttctgcaatttatattttttcatatcaacgctagaagaatcaatttttacaaatttcattatacacctcatagaacttttcatcaggaatccaaccgcgtttaaattcattaaatgctcggccgaataattttgaattcacagttatattattaactgatttccatttagcaactcccgttcgtttataatgatcggggtcatatttcgtgacgtaccattcatataaatttggtattaattttacagcctctggatttgtcgctgatttattataccatggtttcgctttttcaagttcagctgcttttgcagtagcagcaacagtatttccaacaccaactaatttttcagttttaatgcgtggatcattaacatgaagacgaaaaacttcgcccttttcatttttaaagcatgtcatgcctttaattccaggaagcttaacacctttattaacaccgcatcattcctttgggttaaatgatcctttaattaataaggcgcatttacccgatttaactacttctcattcaacaactttatctttcataacgttttttgaccattcagatactgctctttgatggctaaattctagcaatttcactataatttgcactagaacgtaaactatttttcagtgtttcaacattctattcatcgcatatgccatttcacgattacgatgaattttatatagtagaaaatagtgctaaaaagtgttcacgaaaagtcatgtttcaccaaattttcgttatcatcagaacctcccattgatcttggaaggatatgatgaatttcacccttaaatttagaaacgcgggttttaccccgcactattaagtcattatagattttttcgtaattcacctactgttatccatttaccattaatctgtacttcatcattttcatttacgataattgtatcgccatttagctcgaaagtaaaccactcgccatcttctttttcttcaaacgctttttcaccgagaactagaccagtgattgcgcaaatatcaaatagttctttgtttttaagcatatctgcataagacataccccaactgttgagaactttaccacgcaatggataaccaccgtgaagttctttatcacgaacatcaataagatatccgatagccgaatcaccctcagtcaagaaaagagtagtatcagcatctttaccgcaaagattcgctttgatatgtttatgaaccttagctttagaagcctttttagctgctttagtttctgctgctttttctgccgccaatttacgagccaaagcagcttcaataatcggcattagaattgcttcattatttagaatatcacgtgaaatctttttagcatcaagttgaatatgactacgaatttcgccaaatggagaagtcaaacgctctttagtttgacgaatcaatcgcatgtttttcatatcacgaacaaacataacgatagtcaaacattctttgacacgtgctttagtcacatcaattttgaacttacgtttgatttgtggaataaggtcttcacaaatatcatccatagcgcagtcaatgtgatggccaccattcttagtatgaatgttattgacgtatgttaattgacgaaaaccatccggtgaacgaccaaccgcaatagaacaattttcttgctcttgaacaatagcatgttcatcatactgccgtgcatatttcttaaaattgccctgaacctttttaccattaaaggtaaattgaatatcaggataaactacagcaagtgtctggagacgatccagtgtaatgtcaagataaacttgggacagctcattagtttcaaatgacataaaatcaggaatgaaagtaacacgagttcctttccattttccaggaatatcttcccatgatttattttccatgccatttgaacaacgaactacaatattattttgaccgtcgccagtttcaccgacaaacatcacagaaaaaatgtttgtcaaactagaaccaacaccgttcataccgccggtgacgcgttctttatcatcaccaaagttaccacctgcttttggaatagtccatgcggcaacaggaccaggaatttcttcaccggtaggtgttttaaccatcgcttgtggaataccgcgaccgttatcttcaactgttacttgattgtttttaatagtaacattaattttattcgcgaatttaaacttagtacgaataccttcatctactgagttatcgataatttcatcaataagcttaacaagaccaggtacatactgaacactttcccatttaccaaacataaagcgctcatgcgtttcattagcagaagagccaatgtacatgccactacgctttttgatatgttcaatatcgctcagaattttaatttcattcttaatcatcacttatcctcgtttggtttcgggaatattatactccggtaatcataaagctaaaggcccgaaggccttttatttaaaacgaatagttgaatccttaaagaacagcccagaacatactgttccttctactttctgcccggtaggtccaatagcacgaaatccagtatgctggaaatcattttcagagcaaccgaaccaattatatccagtgatttcaatattagtaaaaccacttgaagacaaaactttggttgcattaatcagcatcagtactattaattaaagacactgctaatactaatgctgcaattgaacgactaatatatttcataactaccctttaagcaagtcgtaaaatccattattcccatgcttaggaagcggaaactaaccgaacagccagccgatgacaatcaggacatacaccagtatctcttccagaaattttcttgattttttcgtattcttttgcacagtctttggattgacatttataatcataaagcggcataattattccttaaagtaagctttcaacatctgatataaagaccacgcctgatcattattttcaatagtaactttcatgactgggaattctgtgaaatcttctatttgttcttgctctttctcttcctgctcttgctcttcaaccgcctgatatggattttccacttcatcaaagaacccagcttcgttagtagagagccagataaagttttcgtcaaggatatcaccgccggcacaacgtttgagtacacctatggatgtcataattttagtaggacgcccaagataatcagcatctaaaattttaaaaggctccatacctaaacgtcgtgcatagattccgttatcagtatggtctttaataaaattttcttgagcttgtttatttttaaattgataccatttattaacttcaaatttaatagccattaataaatttccttccagtaagttgtgccgtcttcagtaatttcacgaaatacaccataaattggctgtttatcaccgactttctcatacacataaacagaagtcaagtgagtaaacttgctagtatgttccttttgaactactaccaaatttggatcaaataatacatcttcaaattcatcattagtgcaattctgaacaattttacgtttcattacaatttcctcattaattgaacagtggagcgatacgtttcagaagagtatcaacacctttagcgaattttccattttattctccaagttgttttctgtatcagtagttgatattgatatagtaccataatcaactactgatgtatatagttttatgaaaaatttaaactttatgcatagagagcattgctatagtgtttaatccaactttcaggaatgactttgtatgttcctaaaaataccacgttgtacaacttaacaccatcttctacccattgatcggtaatgtatccacacatagcgcgagtataaacaacccttccatcatctttaataaagttaaattcacaaggagcaatgaacttgatagcctgaccgagtttccacttaaagtctacacctacatgcgaagtatcaatcgtttcaattcctttagcaggaacagcttttaaaaacgcagactcaagaaatttcgcacgaacatagccaaactggggtttagactttccatctttaggaatgatacgcacttttacttcagaatcttcatctttaacaccatgcttaagctgaatgcttacaacttcgaccaattttcctgctgctttagaacgggatttatcagatacacgagctaattcaccaatattaataatcatagttatctctcacttgttaaaaagattttatactccacgggaccattatactctggtcccaagagtttgtaaactattaattcaaaatagctaccactgcactacgaggaactacggagtactctccagcatgaactacgttcagaagttcaacgccatcttccaatccattggtcagttacccaaccaccaatcggattcgcaaatggacgacgaatgtaaactgccttacacaacaaatcagtcgggtcttcaggtttttcaatttctgtcaatagattaaaatcttcttcatagatgatgaatgatgatacccttccataatagtttctaatttccatgtacactgtaccaatgagaattccactattaacactaatgacagtaaaaggatatccgccagttgtaccaagaagttcccaaaatttgctatcagtcgtattcgacattgtctgaaaatcaatttcagatggttttttcatttgatacgcagtattaattttaatcataattttctctttagtttaaggtaataaagccttttagttcggcataggatttacggaacattacttgatgcccgccaatgataacttggtcatctggtacttcgtatacagcaagataaaatcctttcgaagctaattcttcacgctcttctcgtgtgaaccatttcatcatatcatattcgctagcaaaagcaaaatgataaagagctacaaaccatccgggaatatgatattctactccaacataatctttcttgaacttagtattaattacgatattagcatttttaactaatagtttgtcttcgtgcggcacaggaattcttttattatcattactatgatgcataaaattaggtctgtcataacctacatgtaataaccactcttcactccatgaatctattatacttctatacggcgttatttgaacacaaagatctcggcgtattgttatagcgtcttcataattaagaatactaaacgatgattcaacacgataaattttcattttattatcctcagtagctatggtgttatagtaccacaactaaccgaggaagtaaacaactttttatcgttttgttggaagagatagaggatcgcattcttcctctgatggagcatcttcaagacccatagcatatcgcaaagcatacttcatcatcaggatgtctttcgcacagtcatgaatagaatcatgtgcaacgaatccatctaaagttccctttggaagaggacacgttgtcatatcacgaacaagcagaagtgcttcaattctagtacgaatatcacgctgattccaaaatttacaaggttctaacttaaatgtatcaagctcattctcggaaacgccgttaagacgttgaatatcgcgaataagatcgactaaaattggaaaatcaaacgacattccacggcaccagccttgagatttccaaggatcgatattatgtgcattgatgtaatcattaaattttgcaataccgtcgatagtgcttacatcttcatcggatggtgcaatattttttcgagcttcaggagattgattcttccaccattcgatagtacttttagtaaaaagacggtgtcctttttggctttttaaatcaaatttgattttaatgccacgtgaaactaattcatcgaatgtttcaactacttctggattagggtcaaaagcaattacagccaaatcaataaccgctgctttttcaccacttcccattgtttcaaaatctataataaaatcaaacattaaattttcctcgctaaatcacgaatttgacctacagtatagtcttgaatataaactttattaataggctcatcaataaattttgccatagattcaatatctttttgtatttcttcaagactgtatactatctttgaagctttttcgcgaatagtgatattttcaggacccggattttcttgaatgacaactttaacatttgtcataagagatttaaactggtaccaacttaattcaatcattaataatcgcctcataaagatagctaatttcgcctaaaacataatcattgattgtaacagttttaacttcaccgcaaaagaattctaacgcaattaaatctcgttcaatttcttctaattgaagcatcaacttactagattcaatttttacagtttcacgatttttgctataagctatttcataaatttcgcttactttatcttgaagaagataaaactgatctttagttatttccacgaatagcttcctcaaatttaatcatacataaaacacatcataacgaccacgggtgacaccaacataaagaagttgttgagctaattcaacatctgcataatgaatacaaggcgtataaatgaaagcacggtctacagacataccctgcgctttatggaatgttgatgcaggaagtgctttcactttactaaactgtgatttagcatcccaaaaatcactccacggagcttttccgcctttgttccaatttttataagtttctgctgttttagctaaaaataggttaaacttatacaattcttcgtcagatgaaattattttaatcttttcacgataatattcatcatcgccataagtttctactgttaaatcccaatgacgaattagatattctccaggaacaccacgggctttaacaaacgttgatgtatactctgcttctataatacgaactaattgtccgttattaaaaataatttctgacacaggctttccatcaattttatatgttttaaataatggttcctgcattacaataatttcaccgacaataaaatctttatcagtttcaaaaatctttttacgaataatgctatttaacttgtcaacagatttattcgtaaatgccattacgcgattttcaaacaaatcatctagtgatttgacgattgaaaaataatttaccataaaatcgcgtaaagcggtatcaccagtaaatccacgtactccatgcccgtcaacaactttatcataattccacttaccgttgcgaacgtcagtagctacatcaataataggagcattactgcgtttaacttcagtgagttcacactgataaaaatctttatgtgtaaagaatggactgatataagcagtattttctcctggttcaacaggtctgatttgcttattatcccctattccaattatagtacaccaaggtggaatagttgaaagcagaattttaaatagctttctatcatacattgacacttcgtcgcagattaatactctgcatttggctaaatcaggtacttctttttgttcaaaaagaacattttcttcatatgttactgggttaattttaagaatactatgaatagtactcgcttctttccctgatagttttgaaagaatctttttagctgcatgtgtaggagctgctaaaataataccagttccacccgtagatattaaagcttcaatgatgaacttagtaagagtagtcttaccggtaccagcaggtccattaatagttacatgatgtttcttttctttaatagccttcataacaatgttaaaggcatttttctggccttcggtcaaatcatcaaatgtcatcgtaaattccctgcaattggtatactaacaatacgcccagtatctaaaattcgctgatataatctttgcgtgtctacgtcaggcttaacatgtttaacttctattttattaaaccaaaatttacgtggagtctcaactaatcttggaattcccttacctaaagctaatcgatactgctctttaagagtggtaaatactttatcagcaatcttccattcaaaaaatacagcaggacgatgttcatcaagcggaactggcgctgtaaatccgtctttgtctcggtaaactatcgcatatacataaaccatattatcctcggataagtttaaaaattgaacaatttagcggatatcctcttttcagtttaagtttatcaataaaagacaaattttgataccgctctacaccttgaataattttatcacacatatcatattgcatttctgcttctgacaactttttcacaattttccaatccgagcctttaagaagaacgttcaatttaacaacttcagcgccttctgctatgcgagaaccatcaatacgtgctttaagtgctataattctcagcttaatgtcagaggtctgttttgatttagaaagctgagaaatgtgttcaattcgattttcacgttttttctgtatagctttaatttgattataagtctttttgattttagcccatttcttttcatctaaatttagtttatgaacttttttcgcagatgaacgaccaattcgcaaagcaaataaatcacgcttttcaatcaactcttctaaagtataatcagaacgaaatgtattatactttttctttactgcaataacattccctttaatgtatccaacgttattatcaaaacgttctaatgataatttctctccttcaatacgattatcaaaaggttctcccgagtaagcacaaactttttgatctaaaatgttcttaatgtaattgaagtctaagttaaaatctttagaacgtctttttgcagatgcctgagtatgctctaaacgacgtttaattttacgaatttggttattagacagcttcatatttttctcacatcttacggacggttaactacttatactataacatttttactttaacttgtaaacaactttatgaaaaatgctttaaaactttcatggtataatgaatctaagtccttccattatagattaaatccttcaaaatcaagagtatagatagtgtatgttgaacactttttatactcatatctatctgcaattctaaatacacttccagctggtatcattacttcttgttcatctgaaactaattccatattacgataacgatgactatccggaaacttaaagtttggattgtattctttacagcgtagagcttttatagcatactcctggaaattgaataccataggagctttgaattcaaaaataacttgtgtgttatactctaaaccagaagcaaaatgtagagctatatttttatcatatgaagctgatacgactttatcaaatgtaataatatcaattccttgatttaatacttgtttagtctcagctggaacacctctccaaagaggtttatcgtttggaaccaaacgagatttgattatttcatttaaccaagaatggtcatctggtttattagtaatacaatgaattaaaagttcaatttcagataaattaaacccttcagaaagtaattcttcacgaatagaagcacgcaccgatgcatccattgattttattttaaaatcttttagttgcattactgagtatttcattcaactacctcaatatcataaactttaaatgttccaaatgaatcgtgtaatttttcttttgaaatagaagttattttatactttccaattggaatcatccattcttgttcacgcacaatcatcattaagttatcagtacgctctgaatctaatccatcagtatcttcatacgtgtacttaaactcagtattaggagaagaaagtataatatcgctgatatggtcagaataattaaaagctttatcagtttttaaacgaagtattgtttcagtgaaatattcagcataagaaaaagaacacgctgtatgcaaactagtagtaaatgaatctaccctgttcgttgaaaacacttctccaacttgtaaatctttaatgagttcttttgtcgattttgatataccacgatataattgataaggcgatttagttaaatgctttttaatgatttcatttaaatgcttatgaagagcttcattctttttggcttccatacattgccaaagaacagactgctcaaagtcagtaaatttttcacagacctttttatacatatcatattgaaaatcaacgctttcagcttttatagataactgttcaacatctgcaagattaataatcatgatagcctccgtatacttcagaagctatcatatcatcgttagaaaggaaagtaaacaactttttgaattattttgcccagggagcccaaggcggagggtcaagatggtatgaagctagttcttctagaagagcatctggggcttcaattccataattctgtaatactatacggtactctttcttataatcactagaatcattctggttattcgtagaatgattatcttctaacatctcaaataaatccatattaattcctagcgataaaaaccaaatttacgattagtttcaatgatctttctttcttcttcggacattctccagcgtagtccaacatcaaaatgagcccagaccatcctaacaaatgcatttatatctttaatatcttcaataatgaatgttttacttttagaaggttgactcgctaatttaactttatcgtcttcaaacatgtcaaaaagaccatattcatgagctcttttatagcctttaatagttaaagcttcagaagaagaaaatggtgaatctgtattctgcaaaatatcttcagtatgctttataattagaataatattttctggatattttctttcttttatatctttaattaaaaaatccggattttctgctaaaggaataattaatgagcaatttttatcaagtgtatttactgatttaccttctttagacataaattctattgaatataattttgctacttcaatcatgtgatttccttttgcctactaatggaccgtcaggaattttattttcctggatatatttctcattttcttccatcattttactgccaattttaagaagcaaatccattgcttcatttgcttttgctttagcttcttctaacgtcatatctttgttcatgatttatcaccatagatgtctctcatcaatttaagcgctgagcgttctagtttcttttctttctcagcactaatcattgatttcatccattcttctgattcattctgcatttctttatttgcttgttcaacccaaccgtcatcaatatacattgagtttggtctattgaaccattcaagcatcttcttcagaactttcattcgttttacctaaaacaatagtaggagcatcgtcaaatttatgaatttttagcaaatttggatttaaattattccataaagaggtaataaaatatgatagcgcactttcgtccgtaattataaatttatttcctttatctattttccaatcatatattgaatcatatgaatagaaggataacggtttattattataatatgcttcagcaacatcaatatagttagatttagtaaatgcttgaattgccataaatggagaattttgtacagtttcaataattccgatctttttaagttttatttcaacatcttctggaattggcattgaaataaaatcttctactagatacatattattgtcatatcttttatcaatcattactgctacagtaattggaacatccttgacaaacgccatactaatactattgatagacatttcaaacaaaattgcttccataattttcctcaatcacaagatgtagatgaacaactagaatcacaagaacttccacatgaatcacctgcccatacatgaacaggaacattagtatcatatgaatcagaactagactgtgtattctgtgtgttagatgatgtagtaggtgttgaccagcgccaaggatttttataatattcttgggcttcttcataagtcatagtaactgcttctactgttccatctcccatataaacatattcaactacagttaaaggaaggtagtcatttgaaataggaactacaccttccccaggagttgtagagaaaaaatccgtaaagaaacttttaaaccaattaaagataaacattacaaaaagcctcttttgaattcgacttgcttctcaccataatcatatcgaatctctacattaaattcgacagaaccatctgcgtacatcataaatgaatgcacaacaacttctgtagaccatggttgtagttcatatttcttcattacatgtcgtgaaatgataatatctaaatcttcatttggtttaatccaacgatttaacatagtactctcctctataagataattctattataccatactcattttggaaagtaaaccatttaaatgaaaaaaggactcccgaaggagtccttgagttattaaccagttactttccacaaatcttcatttgcagcaatccattcagtacgttgattttcttcatatactgtagaatatgctgctttttctgaagggaatgtctggtaatgagcgccagaaattttgtgaacgtcagaataaagagggaaagctacagaaatttcctttccttcaattttcttatttccatctaatttctgctcaaatgttttgatattaacataaccgcgagtactagccatgtaattctcctttatttaaattacatgattatttatacatcttcttttctgaataagtaaattaaattcttaagagccgaacttgttacatcatattttcctttaagcgcctttacaaccgggcctgttgctggtttacctaaagaaacccataactcgtgtatttcgcttttaagtggttcatgccaatgcggtgcttttctttgcgcccttgaagttccttctgaaatctttttatttccgccattcgaataaaactttttcattacttcagattgccgagcttttctttctgccctattttgggctatacgttgtgaattcttcatccgagtttttgtttcaggattgtttaatctaagtttatgttctaatcgttgttgctcagtccattttcttccttctccaccctgaccaccaggagaaatattaatgcaagtatctgggtgtttacgttttaatgcagatattagctcacgttcaacttcatatgatttttctctagaaccatggcattttgaccatcgtattttataattaaatccatacttacgatatatgttccatagtattttacctgaacccggatatttatcattatatggatttacaataaatgattcatgtttcccagcgtaccaaaagacgcctttcggcgtcctaacttttactatatatgttctataaaatttttgtttaatatccattatcttgacgttcaaaattatgtttgttcttcagataataaagtttaaagatttcttccgcattcattccaaggccaacaaacatatttaatacgaaatgaaatatatccacaagctcaaatttaatttcgagctggtcttcgggggacatttcatcaatgcgtttttcttgcgcttcaatataacgtgctttccattttttccatacagcagaagcttctttttcaccacgtgacatttcaccaagagaagtcagaagttcgcggaattcatcatcaatacagtctttttgttcacgcatccaagaaacaacatcaccggcagtttctaatttatctggatgatagcagtattcgcggacattagccaaacgaatctgtaaaaaccgctgcatatcaagcataacttgcagcggatctttttcatcaccgagaatatcccagtattcattttgagctttatcaacaccttcgatcaaatgagcacattcattaaagtgagccattagttttcctttcaattcattaataagttaaataattatatcatttgagtatgtaagcaattaattaaaaatatatacttcatcagttccattcttttctttggaatgatatatgttaaagacgtattttttattaagatgctttacattatattttttagaccattctttaagaagagtgttttcctttccgtggtgttctaaaacattcgactgcccaaattttattcctctatcatttaaagaatctaaaagatttaaaaggtctttttcttcatcttctgaccaaaatttattataatcagcaactgttatgagatacggaggatctacatatacaaaatcgccgtctaaaattttaacatctttaaaatgcaatgaactaaagattattttatcacaattttgtttaaagtgattatattgtttttcactatttttgtttatagttctttttccaaacggagtagtaaaatttcctttatcgtttatacgaatcatattactaaatccgtgaaaatgaagaacataaagtaaaagaggatctctagttttattataatcttcacgtaatttcaaaaactcttcttttgatgtttttgatagtttgtattgctttattacttttaaaacgtcatcccatgatacattaataagacgcttatacatttcaataattggttcttgaatatcattggccaatacagggccattaacattcaaagacactgataaacctccacaaaataaatccacgaatctgttatattttggaaagtgagatttgagttcaggtaataatgattgtttattacctgtatacgcgatagctcctagcattatattctctcatttattgcagcaaaaatgaattatacaattcttcatcatatgtatttgatagtaatactaacacgttttgcgataaatattaatttaaaggaggatatatatggtacaaaaattaatggcacttgttaatgccataaaaggtaataagaaacgtatagcttttactatttctactatggtaggaattttactctggaactttattttatcacctgttgcaattgcacatggtgttaatattccagtagttactcttgatacattcgtagatttagcatttgctttagttgggttaatttaaatcttagcatatttagatagccgcattttagccattaacccctgggcaatattatttttcatatattccataatttgttcaggggttgcaccttcctttctaatcatatcattaacatcttttgatttccagggagatttatcccaaaacataaccctttctcctgcatcaactaatttagtcattcgtttaatagtgtcagggtgacgaggttcattatctaagacccacacacgtctatctttaaatggaacaacttctaggtctaattgaccgcccgtaatagctataccattttcaataaaaagtgaatctataggtccttctagaacatatacatcaccatctttaactcgttcgactccatagatttttgttgcctcaggataagcttcgatggtgatatatttttgaggagcatctttctttaatgcacgtccttgaaaagactcagcttttccattagcattataaattggaataacaagacgaggctcagaaatttcctttttgtatgttcccggtgctatgctattaactaatttaggccattcggttgtaaaccaaagatatttccatttatcctttggaatacaacgagcttttacgtattttataattggatggtcttccgccagtttatctaatctaacacatgacggaagagatttaattattttcttctcgggttgtttaggaagttctttaggtttttctattggacgacttttacctttttcttttcttatttcaaagatatactcacgatataaatcgggttcaaactcctttaaatatattccgattggtgcatgatagttacagttataacaatgaatatttccttcattattatcaccataataccatccacgggctttattctggtcggtttttgaatctccacaaacagggcatctaaaccgtaatttaaaagttgaactattatttacttgtgtgaatttaggtaaatgagctaatgcacggtatgcaaactcattatcaatccaaggtattgatgacatttttactcttctttttctttagattcctcttttttctttttaggaatctgttcaggacctttatttactacagcgcctgatgttgttccaatagagatattttcaggattaccacctgaatctccagcgaccatatcttctttgataaattctttaaatgttttcatattaacctctattcataaaagcattaaaaatttggtcatcaatagaaacatttactttaggctgtttttcagatggcaattcatatccacatattacaattttatgatcaatatcaaaatacacagaagcaatatgattaatgatattttcagtaaagtctaaatcaacatcaatatctttttgaccaaagcccaaaggataaataatgcgagtaattcgattatctttaacaaagattccaccgacatactctgtgctgcgtttaaagtctacacgccgacgaaatgaaaaatattcaggctctttatgagctcggctcataggacacaacgaataactagaataagagatgtcaaatcctacgccttcaatatgaactaaattgtcatgattaaaccaattataatcatatgccaagtccattagattgtcatatgtgaaaagcaccggattaacatcattggtcacaagcatataattagcaactgctataatttcattatttttaacgaatacaaaccgtgattgatgcgaatggcctggacctgcgttttcacgattaatgatataacactgggcacctttatatttgtacgtgtctttacacttgtgcatttgataaagcattattcacctaccacttcagcgatgatatttttgttattaaagtttttatcgcaatacagaacataattatactgcattacaccaccagacttaagctgtttttgcacttcagctttcatttcaggacgatcacgcttaacgatattcataatatctgcttcaatttgagtttcaacctcagtctgatccgcagtcatagaccattcgcacaaatctttatcataacctgccatagcaggctgagcagcacaagaagctaaagcaaaaattgtagcaaagatgaattttttcatgataatctcctcagtagtttatgtttatatagtatctcaatttccaacaaaagtaaacagttattttaaaatttctgcgtaatcacatgttacaaactgtttctctaacttgacgattttacgaaagtatcttttgcattggcgaatctgcctcttcgtagggcgaacagcaaacttaataaattccactcgaccaaatggagggctttcttctgctggaatatctaacaccaattcccacgtatctgcaataagtgctttgaattgcgtatttttcctgacgttatacggagtaggtttaaataaaacaatatgcatattatcctcggcaatccacttcacatactttcttgtcatcaatgaaagctttaactaatgctttattaacttcagcatattgagtagtagcccattgaacgtcatctttcatcattgtgatttctttagtaaacatgctttcattcttaaaccaccccataaaaactacctttaccaattccataacaatctcctcatttaaccgacaagactactataccatagtcttgtcagcttgtaaactaaaattttaattcattcgccaaagcatctaactgagctcgagtcgattcatttctttgatagcgattctgctcagcctgaatctgttgtgaacctgctacttcgttcacttcagttggagtagaatcttgttcaatttctacccatttttgatttcctttttgaacacccatcaaaaacttattccacttattcttatcaccatatcgtgatttgatttgcttaatgagttgttgttcagcagctgctagctcctcggtttcaatgaccgcaagcataaaatcggctgttgctggaagaccggcagattctgcaatatcgctcatgttaacatcggaagagtcccaagcttgtttaccaacctgtgctgcagtccaaagaacagtttcggtttcaacagccagagcacgtaattcctctgcaatagctttaacagttgtgtaactattttctgaataaactctaatgcggcaagatttacaaatacctagatagtcgacaataatgattgttggaacaaaattcttcttgagcttcaattcgtttaaaagtgatcgaaatgtattagcgtctgctccaccagtaggatactgtttaacgattaaacgaccaagagtagatttctcacgccatttttccatttttcctttatactcagcgtaagaaatatgcccatcatcaatgtcatcaagagaaacatcaagcatattagcgtcaatacgtttagcacagacttcttctgccatttccatggaaatgtaaagaacattatgtccgagctgtaaataatctgccgctaatgaacacaatcctaatgatttaccaacgttaacgccagccattaaaacgttcagtgttccagtttcagctccgcctttcgtaattttgttcagaattctgagtttaaatggaaccttacgagctttattcatataagatagccaacgtgcttcgtagtcatccatccaatcatgaccaacgtaactatcaaatgaaattgataatgcctggcgcatgatgtcaggaatagcaccaacatccggcattttcttatttcgtttttccggaggaagctcagcattagtttgaatttcgattattttagacgtagcattaaacatcgccctttgctgaacatatttttctgtttcttttactaaccagctgtggtcttccggagaatcagccagttttgaaataagtgtttttacaccagaatattctgtttcagtaaatgaactattttctaatgcaacatttaacgcattaatagatggaacgctatggtactcattaacatgagatttaattaatttgaatgtatttttagctggaccactttcaaaatattctgaatccatatatggccaaacttttgaaaaataagcttgatcaaatatgagatgagaaagaataatttctaccacacttactccttaaaagaatttaaattttttctttgaccttttattaaatgcatcttgcagttgcattgtaatacatttttctacatgaggagctaactcagcttttctttcttggtcaagaacagcaaagtccattacaacctttccatcaacccaatccagtttagttacatacactatatgcgtagaaccatcttctagtttaatgacaatctcctggataacattttccatagcagatttaattatcttaagagactcattaaaaagacgttcttttctttcttcttccccctccgaagagggggattcatcgataatttctagatctaaatctaaatcatctttattcattaaattcttccatatcacttagctgttcgaggtcagtttctaaatcagcagctgatttacttttactttctggagatttaaatttttcaacctttgagttaatcaattcatcaacttcagcttcaacaatttcattactatcaatagcacctaactgataagcacgtttaatagcatctcggaatggttgatgcttaaataaaggaccccagaatgtagtgcagttggtatcttttgcacgccaagatttttcttcgcgaatcatctcgccagtttcttcgtcaagaaattcacgagcataccagccatttttaggttttaccacgaatcctaattctagagccatatctaacaatccagaataaggatcgataccaccgtcaaatttaacatcaataaagaatttacttttttctttaacggtacgagatttttctacatttagaacaaattgatacccctgaagatcagaaccatctttaatctggcgtttaccgataatgaatacggtatcagccgaatacatcggtccagtaccacctcccataactgttttactaaacatttcttgtgtttcgtatgtatggttaatagcaatacatggaatatttttagtactaaaatagggagttacgatacgaaataagcttttcattgttttagctctagtcatatcactaacaactttttcatttaaagcatcttcagtttctttcttagaagctaagttaccaagtgaatcgataaaaacgactaccttttcgccgcgttcaattgcatccaattgattaaccatgtcaatacgtaattgctcaagtgattgaaccggagtatgaattactcgttctggatcgactcccatagaccgcaaataagcaggagtaataccaaattcactatcataaaacaaacatactgcatcaggatattgacgcatgtaagatgacaccattgttaatccaaagtttgatttaaatgattttgatggacctgccaaaattaacagaccagattgcataccaccagtaatttcaccagaaagtgcaatattcatcataggaatttttgttcgaactacatctttttcattaaagaatttagatgctgttaattctgcagtcaatttagaagtagaagctttaatcaaacgagattttaaatcagacatataataccttatagtagtgttcttgttccacgaaatacttcgagcattcttgcataagatttatttggaaatccagcttcaattgctaattttttaagcttaatagcaccggattttccagagttttcccatatttctttaatcttttcaatgttatcccacattattgggtcacgtttatgtgatggtttatttctattataaccatatggattattaagcaaagcttcttttcgttttgctttagtttcttctgactgctttttgcctagctgggctttccggcaaacatcactaaatccaagatgttttggttttcctttttgagcttttgaaattttttctttggtttctgatgaaactctataaccaattcttctaccaccctctccaccaatttttaaattataagtcataggatcatttaccacatctattgttactaattctctttcagcatcacgggctgatttaaaatctttataaaacccaagaatgcttaaattaaaatttttcttaccatactttttcttggcttgtgctaataaagttcctgatcccatataaccatcatttaaatcatcggttgaatgagttccatagtaaattttattattaactaaattagttataacataagtataattatatttcttttctttatgtcttttcatgtcattaatcaactgctattcgatgaacttctctcttttctaatcaatataacaaaagaagtcccaaaaagcaaacagacctaaaccgataataagcaataaaggtcctaacatttattccaccggttaaagataaataactttctaataatagttcataatttttataaatcaatagcttttttgaacgcatcttgccattcggctttctttgcacgggttttatttaaaatatcatgttgaatagaaagcatctctttacgcaaaacatcactgtgttttaactcattgactctatcaatgagttcagcacgattatttacataaaaacgagcatcattaataattcgatgtttggtatcaaattcttcgtcaattagcatcactgcatcagatgccattgtttcccagacgcgtaaggtaataaagttgtcattataattcttgtcaccaataattaatgcagcaatagcttgactattcttttcagataccatgttcataggaatttttccagtgaacaccggagctttggtccaaggatatttaggatttttaaactgtttttctcgtgcattgccaaaaaactcaatatttaaaccggtgtcaaataagaattctaccatcttggattcgcgttgaccggaccgaaatgaaccgccataaataacatccaaagttttcttggtaggcttagataattgaaaatcgttcatatgaattttatattgttcaataggaaaatattcaaattcaataacattatcaactttcttatgcgcagccttagcaatgtctaaatttataccttgggaaatcactttaattggtgatttaattaatagctcttcttcagtgtacaaatatgcccatggtctatttttaacatttggccaagactgcgaaaacggcaaacgtatatctgtaaataaataataaattttacttttgtattttgccataaatttttgcgcagataaaattgctaaattaggtttaccgccaaaaaagttaatagaagaattaacaactatcaaacggtcataatcattaacatctacttcatcaaaagatttagtgtaaacaccatttttaagagaaataatgtcgacattaagacccatttcagaaataactttaaaaagataaatagtttcagaagatggaacagttttaaaattaataacattattacccatattaattatagcaattttcatattattccttttatgttaaacgattaagcgtattttcctacataatcttttttcgaaatatgtgtttcgccagttttccaccaatgatcaaccaaataaaaatgacgagaatacacatgtaggcttccaacattccatataatggaacctgctttatactggcgagttgaatcacctgcattcaaatcagatactaatttatctaatacgtatttttgccatgcataatcattacggaatccgaagaccacgtcatttgagcgcatgttaacaaccgcattgattttcttgtcacgaatcaggtattgtactgtattcgtgcacatgaaatctgacataccatctttattatagtcaaactgcatagatggacgagtataaatcatgataccacgtcgagaatcaggattttgaccaagttcagctaaacacatgtcatactgagcatagttatcttctgaccagatagcccaaccataattcgagttaatttcacctttagaagatgctacttgttgccaaatcttcggtgtttcacccggaatatctttaacgaacaagcttttagatttataccattcaagttcacgctgaatgtattcatcattaagagcgccaaaaataaacggttcatctgctacaaatgatgcgccaataatttcaatagttttaacacctgttttatcaactacgaaatctttttcttttaatgcaagccccaaatgaagacggatttcttcaactgtcatagagtcactaatcatttaaacctcaattgatacattcatatttaacttgtaacagtaataaactccaacctaaaataatagttggaatcataagaggaaccgttacactatagtatatacttattataatcatcaagattaaaagcaatgctgctataattttgcttttcattccttctctctgatgataattacctgatttggctgcgcagactttttagtttcacctgcaattgaccaaataaatgtaataaaccaaccaataattgaccagttaaacagtaaagatgcgaaaaagattcctactgtcgattttgacccacgcatcaaggcgataaaccatggaagcatgtatataataatagccaacacgcctgaaactaaaaccataaaaattgaacctgctactaaagtttccatgttttcctcacttaggtcaaattttttacacatgaattataagaattcactacatactccatcggagcgtttttacctgtacgccactggtaattattagcccaatttgcccaaagctcagcgcagtagttttcaattttttcttcgcgtgtaattacatctgaattacgatatgcttgagcagattcatctggacgaatagcttcgtcaaaattcgcctgcatttgttctactgtctgttttggagcttctttataacacttgacattaggattataaaatttgcttgaacagtttacaatttttcctacatcagactgatttactaccggtccttgagctacacaaccagcaagacctaatgcaataaccaaaatagcgattttcatttcattctccaaatccgtatcagtagttgatagttgtatagtaccatggaagaacagtcttgtaaacagttttgtgaaaaaatttttagggaatccaaagggtccagaatcatctctttttcataagtatagatttatattacttgtatgaaaaagggacctggaggtcctagatttattctatcagccaaacaggaagtctaacgaagctttttcttcatagtccatgccagccgattcacacatacccgcaagcggtttaacaaacgatttttggaacaaagttgagtggtcaatccaagatagcacatcagaacgaatttcttttggaagttctgtacccgatggccaagcaatgcacttgtcaccaaatggatttccttcacgtaatggaagaaccattactttatttccatccaaaattggagctacacctaaaccgctaacagctcgacgataagttagcacaccacgaatatggaacgggcatttaaatcctggccaacctttatcatcatatttcgctatatcgttcgcagtttttacttcagcaataactttatagtcaagttgacgatattctttctcgaagttcttgtagtattcttggacagactcttcaccttcctgaagaatacgacgaatactttcttcgagagcttcttgcactgcttttggtgttgaactctgctgagtttccatacccatgatttttagatgcggttcagcaaatcgcttatcttccatatcataaacgttcagagcataacgctttttcgctttccaaaatccaccaacgccctttgaaccaagcggagggcaagaaatagcttcacggtccatatgcatcagatgctcgcggttattcatataatcacataactcacgatatgcaacatcaatcataggttccatctttttcttaccgaactgattcatgaattcaaccaaatcgttctgctctttgaatcggtcaagaccaactttttcaataactttatctacgcaaacatataccgaatcagtatcacctgctgcaatgaaatcttcatcattagttccgcatactttattcagatattcattaattttacgagcaatccactgaataccgacttggccgaaaattgtgatagcagtagcatttcgcaaatcatagtaacggaaatgaatattaccaagagcaccataaagactgttaatgagaattttacggttcagctgatttgtattagcaagtgtagctgctttttcacattcttcaatcagactattgagaacagattcggtgtaattcgatagttcatttaagaaatcatcactgaacttaacatatcgttcaacttctggtttagttgaacaagaccctgcgcctttcataataatctttttaatagcttcggcattcatttcttcagcgaacattttctttttccagtctttacgctggaaaaatactttagcgatttcctttggaatgataccttcttgatgtttatcatacatccatccattcggagaacaagaatattcatcactcggtttaggagctgttcctgcgatatattcatgaattggatgaactttaaactgaccacgaatagtttcaggactaatgttaacctggcgaataatgctcggatacagagacgtcaagtcaaaactcataatgtatcgacgtgcaattggtttaggttcaaacacaaatgcacccggaaaactctgtttaacgtgcgaaccttgttgaggaataaccttatgttcacctttcaatgagttaaaaataatagcatcccaagttttaataggactcattacaccagaaaaaggcattttagcgtaataagacatacttaaaactagatcgataaacccacgaattttatcgattgcttgaactgattctacgtcaatgatgttataactaatgtatcgttgatgattagtctcacgaagtttattaataggaccgtcgtatggtaatttaccttttttggtttcatgttgagcaactgattccaaagagaatgacggcaaattagtaaaagcgaatttcttgtacaaatctaaataatcaagaatagatacgccatcaatagaataaatttctttgctaccgtacatattttgaattagtttagattttacccgaccgattggagagaaacgtttcatactacgttcacccagaatcattttaacacgattcatgatatacggaacgtcaaacccctcaatattccaaccagtaaaaatagcaggtcgtttctgttcccaaagattgatatattccatgagcatatcacgctcattatcgaatggcatataaattactcggtcaagaatttcttgaggaacttcatcaccaccttcacagtcaagcttagcagctaactttgcatcccattttgatactgaaccgtacattgaattcaaaaggtcgaaaacataaaaacgatcgtcaattgaatcgtaatgagtgatagcatcaatttcatattctgctttcattgggtcaggaaatttatcaccagtaacctcaatgtcacagttagctacacgaacaaattttcggtcataaacaatttctgaaccatatgtatcacttatataagcgagtttaaaatcgttcataccgagagcttcgagaccgatgtcttccattcgcttcatccaatctcgagcatctttcattgatggaaatttttgaggagcgcagtttttaccatagatgtctttgtattttgactcttccttacaatgcctaaacatagttggaagatattctacttcacgggtacgttcctttccattttcatcaatataacgttcaacaatgttatttccgactgtttcaatagagatataaaattctttcatagatattccttagtttatagcccgagttattaggctcttgatatattatactccaaataaggggccgaagccccttgcttaattaccaatcgtatatttaggaacgagcttccattcatgtttttgtttaaaagaaataactcggaagttattagttaaatctttcataaaagttctttgaccaggaacgatttcaattagtccccaatcttctaatagccatgcaatcgaatcacgacgaacttcatcttcttctgtcatttcaacttgacgaccatccatacgaagcatttctttaaaatgaacgatatagtatagtccttttttctgaagaatatgacaggactgatatagaactttatctttattattagcaattcccatacgagtcaaagtttcttttactttcagaaaatcttcaggttttttaagagtaatttcaatcattttaccattccaatgctagttttttgagttgtttctgttcttttacgttcttagtcacttctttcaaaaaatcatccgtgactaaaccttttagttcttttaatactaaaggaagttttccatttttagtaagaattgatttatagttaattgcatcatttgtattaacttgataccgcttagcaagtaacttaataatcaatacttcggtggaatcttcaaccagttttgcccatttaccatatcttttaccacgaggaactgcagccattagataattaaaatgagcttcatcacttaagcctgatccaattaaattcatagcatatacagctggcatacactctggaaattgtgataatgcattttcaaccatgaattttgaataatctttttgagcaatagagcatttagttttattattaatagctccaattatttcaaaaaattcattttctgctttttctttaaaagaatcagcagcggattggacagctgtccaatcttttgaataccaagcaacttgatgctcgtttaattgaatatcatctttaaataagctcatatcacttccactgcatttcgcatgctaattgaatgaaaagataagctaaatgcaattcagtattagctgcaataccatgatactgattattttcgccgacaatttcgtacatacgaataatactttgtggagttacacgtgaatagatttcttcggcaagtttacccacgaaccacgaataatcagccgcatattttggtgctaaagctctgagttgtttaacatctttatttttgagagactcaagaacatcatcaatagcaccacgatcgttagtaaccagtgataaaataccagcatccaaaacacctttagacgaataactatcgagctcgccaatagttttacgaaaatcaggaaaattctttttaaccaaagctgctacaactttcatatcagctatagcaattccttcatgcttgcagatttcagtcaatcgacgaatcatctgcttcatcatttcaattttatcttcatcagttggttgaccgaatgtaataactcggcagcgtgactgaagcggtttaataataccatcaatattattagcagtaataataatactacagtttgaactataagcttccataaaggaacgaagatgtcgctgagactctgctaaccctgaacggtcaaattcatcaataacgattactttttgacgaccatcaaatgaagcggcgctggcaaaattagtcaaaggaccacgaacgaaatcaattttacaatctgacccattcacaaacatcatatcagcatttacatcatgacataatgcttttgctacagttgttttacctgttcctggagaaggagaatgaagaataatatgtggaatcttacctttacttgtaatagatttaaaggtttctttatcaaaagcgggaagaatacattcatcgatagtagatggacgatatttctgttcaagaatgtgttctttttcatttacagtaatcataatttcctcattcaagttttagtgtaaattataaaggccgaagccctctattaaaaatcgtgggtagaatcagcttcaagagctaccacataattcgcgtgttcaccttcaaatttagcagcaccttgtttaccttttgcccaaagcagaagtttataatttcctggttgcattttcatatttgccatattgataatgaaattaaatgtattttcaccatcataatcaccaagagtcaaagaatatttaacacgggtcagagcagaatcttctactttattaaaaccgttaattacgattttaccttcttttaccgtgatagcaattgtatcaatttgcagaccacgagatacacgcaacagctgttgaaggtcttcagctttaatttcagtaacagcagatgctaccgggaatggaattggtttattaggagcaactactgtactcggatcggctgctggccaaaaaattgttgagcgggcatcagcaattttaatatttccatcttctgactgggaaatttctgcatcatcattaactaaagacagaataccgagaaaaccgttcaaatcgtaaattgctacatcaaaatcaataacgtcagaaatatttgcttccgcataagttgtaccattaactgcgcgagtcataataaattgaccggatttaagcataataccagagttaatagtagcgaaatttttaagcagagcagtagtatctttagacagtttcatgtaatttccttcaattcaaatgagatttaattttataactaatttaataaagcaattaacgattaaaatcagccgcaattgtttccgcaacaatttgagcagcaacaattagacgttcatctgcattaccgcaataatcatcttcaaggcgttcaccacatgaagtcataataaatttagcaccggcgtttagggattctgtagtatgtttgcgcattagttcaatccatttattacttacttcacgatcgatagcttcataatacgcatgacgagcaggtgcagatttaattttgttctgaataacttccattgcgttatcagaaagagacaaaacccatgctcgacgaattttattttggttttgtggatttgattcagaacgcacgtgttttggctgaatatcttttacatcaacagtataattcacagtaattttagtcataatacacctttagtcataataatcagtaacagtccaagcttcatttctattggacattatttttgtatattctgctttaaatgcattcctaagcatagattcagtaactatatgctcttcattagaaaaattatttctcagaatatatcgttttatttcaggaatagttaatagatgctgtccagttgaatattccatgtttttcctccatagagattatactctaataaattaaagcataatctcttataaattaaaccattacagtaaatcgaccaactttcttcatttgaagatgctgaccatattcttgcgggtcatggtctttatgcgaaattataaaaacgttagtgtttttcattgaatttataatattagctacacctttaataccttcggcatcaaatgacccatcaaacacttcatcaagaattaatgtactaatactaacaccagatacgatagaagcaatatcacgccaagtaaataaaagagcaatatcgattcgtgccttttcaccttcactaaatgaagcataactaaaatcttcacgaccacgggatttaattgtctcattaaattcttcatctaatgtaaacacataatccgcttccattattttaagataatggttaatctgcttattaaataatggaatgtactttttaataatagcacctttaataccagaatctttgagcatatcagtcaaaattcctcggtggtatttttccattactaaattagtttttgtcttaacaattttatcaagttcttcttgaagcagtgctatttcatcagcatggtcaataaactcagaagatgctttttctatagccgctttaacttttttagctttatctactgctgcgatcagagattgctttttattgcgaatatcatttgccaacgactgctgggttttaatattatctcggtattcatcaacaagaacttttaaattatcacgatgtgttgaaagctgttcaaacgaatgtgtgcattcagaaactttatctttaattttagaaacaactttatcaccggaactcaattgtgacaaacaggttggacataatccaccttcgtgatacatattaatgactttattatacgagtcaatttttgatttaattaaaaatgcttcttgaccgattttattaaatgcatcagtcgggtcttcgtccaaaacaatattaactaatctttcgttagcttcttctatttccgattttagcgttctagcttcttttgccaaatcatcatacatattttgtagacgagtaaggttgtcacccgttaattttttctggcgttcaacattatcattatatattttaatttgttggataatactatcttttttaacatcaagcacttggttctgcgaatttaattcacgtattagtgctttattaagcttatccatttcagctaatgttcctacctcaagcaggtcttccacaagctttcttcgcgcaggggtcgacaaacccatgaaaggggtataccctgctgtaccaaggacaacaatctgcttgaaactggcatatgacattccgataagctgttcaaattctgcttggaaatctttactgctggcagattcattaagacgtgtaccgttaacggtgatttcgaaaacgtttggtttttgtcctcttttgatatagtactttttctcatcatattccatccacagttcaactaaaagttctttcttatttgtgctgtttattaattgacctttctttacatcgcgaaatggcttaccaaaaagcccaaatgtgatggcttctagcatagtagacttaccaccgccatttcgtccagtaataagagttttttgaaccttatctaattgaatgtcaatcccattttgaccaactgacattatatttttatattttactctattaagtttaaaattcttcacaaaagattccttttaatgtatcttttagaccattctatcatatcatcataatctaaaaagtattcatcaaattcagccatgcaaacaacgccttgtgctgtttttgatgtgatataaattattccaacatatctagaatcttcttcggtataatcaatatttgctatgaattcatcattaatgtcaaatgtcgaaaacttcacagtatgcatccttaatacaagatacagccatatctcgtaatgatttaggtgtgtcattatctaaaatgttgaagttaaaagatacagcccagtcagtgcagacagtaacctttttaatacaattatcttcaacctctaacggttcaaaccagtattcaataatttctttatgaccaatatcatcttttacttcacatttaaaatgctgactcatcataacatttttaaattcatcaaaagtcattgtgttgcctctacatatagctgatttgcatattgaataagtgcttcacggtcagaatcagtgatgtctggaattgcattaatatactcttccattaatgtctgaagcgattgaacttcaacttcttcactgtcatctgactcgacagagttatcaatctttgacacaactcgtaatgaatgcacaactttttctagttcagattcgaacttcgtcagatttttgtctacttcagttactataacacgtactgatagatttgtaaaatctttatagtcaatttttcctttaaatggataatgaattctacgatgccaggtagtattgtttggaataaattccgttcgttctgtttctgtatcaaacatccagaacccacgagggtcattctcgtcacctgcggttagtgtccatggtgtcccaatatatctgacgtttgcagcctcagaaatagtatggaagtgaccagaccacacttctttataagtcttaaggaaatcgggttcaagaccatgagatttcattcctttataaaaataaaatccattcagttcccagtgaccaacacaaaaagaagcagatgaagttttgatatgctcaagaatttcaccagtattttcttcgcacatccaaggaatcaaatcaatcaaacacccgtcaaaatctactgtagtaggcttatcatacactttaacattaggatatttagccaaaagctcagtagaagcatttggatgcattacatttttatagtggagatcgtgatttcctacaatagtgtgtaatgtaattccagcatcatcaagcgtttgaactatttcacgggcaaactccatagttttatgtgtgatcgcttttcgcacatcaaaaatatcaccgtattgaatccaggtagtaattccatttttcttagaatattctatcgcttgcttaattccatcaatttgaataccgcgaatccactcatcatcagctttaacgcctaaatgccaatcacctaaatttaaaattttcatatatcaagaaccgtcattgaaatgcaaaataaaattattgaaataaatccatctggagtgctaaagaacccaatccaacatgctctagtgaatagataaaatgcaagaaaaagtatcacatatccaagaaatatcattatatcaaactccgtataaagctaaagggccgaagccctttattttgtaataatgtcaaactgttctttaaagcagaagcttgaatcttgatgctgatacaaaaattcatatgctttttctcgctcacggtcataaagagctcggtcagatgacagttctttaatacgttcaaatgttgattccatatcattttcatcaaaccaaatgataccgctatcatgcgaggtcaaaggagtattatcaacacggaattttaaattttcgccagtagatttccaaaataccggaattgttccacatgcaccaagctcgagatgagtatattcgagtgagcgttgtaagtatttctggttaagtttactcaactgatatccaaagccagatttactcattcgttcaagcatttcactattaatataacaatctaggatttgtgccggttgattcggcgcgagattcattttatcaatctcacgattaccgtaatattcatacggaatacctttttccttaattgcaataaaagcaggggaacgttccagaccttccattacagtggatttaccagcaggttttaagaatttttcatgaaaatcaaacatctggtaaaaacctttccatgtagtcgtacgaccaatccaacggttgatattcatgttaatttcagaaacatctttccaataagttgaccgaaccttcacaatatccataggaggctgaaaattatatactgtcggtgcttcttcaatatcatcaaacagagaaacagtttctggataccattctttcatcagaactttattaaaatcaccattatcagaatggctaaaaataacatcagctcgacgaacagtttcttctaatcccaaatttcgacgcaaagaaagaacagaatgatcatgctgataaactacaacacgaatagaaggtttaatattatctaaaagttttttatagttattaatcgtagcttcttgaacggaagtagcaggaacagaattaataattagaatatcacaatcatttactagcttaagtgctttatcgtattctttagctaaaataactggaattgaaaatgatttgtggtcatgagaacttgtacgagtaaatgatttatctttagcataaaccaaagttacttcatgaccatttttaataaaccaatcacgttgctcgagtgagaattttgttacaccacaaccttcaagacctcgagccataaaaatgcaaatacgcatagttttcctcttttcatttaataaatcatgtaaataatattttattttctataaaacgctacgaataggcccaaacatatccataagcaattttgctttttccagatatgcactttttaatattagacatgcatcctttaacggatacagctgcgtctttaattctaggaaatactcgtaataattttccagtagtatcatattgaaatactggcacatttcttttctgagttatcccacgtttagatttagacattttttgtttagttgcattagacatcctagagggctttcctatgttatcagaataataatatttccattgaaatcctccagcggttttccttttaccatctacacattgtttaattgaagttgaacagctatatgacatatcttctgcagcatctgtaatacatctatatttgcgaataaaatttccatttaaatcatattgataaattggttttccagcatttcgtctagcgttagacatgtattttttattatcatcatctttccagaactctttcatgctttccgatattgaagaagatacagctgttttaatccatccatacattttattatttagtcttgttccgtcagaactataacacatcatacgaatagctaaagccaatttaggaagtctataaattttaaataataataaatgcgcggtaaaatgttcttctggtgtcaaaagaactaaattagttttatcatctgtaccacccatacatctaggaattatatgatgtgtttcagtatagtatgtcaaaagacttttatcattgcctctgtttagtcctttttcgatcagtaaattatatatgtttaaataattcattttagtttattttaccaaaaaatttataaagcaatataggagccgaagctcctatccacataatacgccatacagaggctcgttagaacttttaaattttatgcgcttatatgttatagttccttctgctttagctttatcatgagcctctttaaagcgtctcatcatttcctctctagaggaacgaattttattataatctatttcagaagtcggatgttcatctttcacagttgccaccattttttgacttgataagaatcaacccacactttcatattagggtctgctcttacaataggaggattaacttctttgattgaactatcacggtattctttttcagaaattttcacgcaaagatgaccattcaaagattcagtaaatcctgcattaattttaagacgcttgaattttacgtgtgtaagcatcaatcatatcctcaatctgcgatctagtagtcttccaaagaatactgatgagttcatcgttatatggctgttttagaatatcccgacttttcttgatagcatattcgtattgagcaaaattattattttcagctgcgatctgagcgtgcttataaagacgattcagttcgcgtttgtttttagacaataacttatttgcttttctttctgcttcaagacggttcttttcttcaatagaagaaataagcttttccacttcatcattaatttcgggtttatcagtcatattatttctctaatataaaataaaaatcatcatctgttaaatgataccgatagtttaattctacaccattagatttaaaagcggtatcatacggattttctggatcaatatcaatgtcaagagctaaaacttccctgagatacattttaagtaaatagggaatagcttcaacttcaggtatttcttccaagaatccggagaggttaatcgttagcctcatataaaaaatccaaactaggagaatcatctacaacacttttcttttcagcccccggtgttctataggttgattcttcgtaatgcgtcattttatcatagatgtcttgaataaaagtttcatctactaacgcaaccatatcgtcgtcacggctgtcatagacattgtgaacgaagtaactatatttctttgcaacttccttacgttcttttttaatacgttggacgaatgcattaaaacaagcttgagttatatacgcatgtgggtttttatatttcgtttcatcaaaattgtgaagccccttaatagaagcttctataccatctgcaatcatttcttgtttccaagactgggtgtatcctgaaaagttgaaacgtttagataagccttctgcaataagcataatggctaatccgatagtatcattctgacgaactactttatttgggtctttattatttgctaattctgttttccaatcaataatagcttgtaaaagctctttattgtttacgtaattatatttaggcttagtttctgacattttcacctcttagctcaattcatagatctattatatcataatatttgaagacctatcttaaagcatagaggatgaatcaatttcaagcacttcatcagattagccgctccaagagctgcatctagtgaatcaaattggtcaacatattcaattaattcgccgtaattagcgtataaccaccattggctaaattcatactcaaggatgaatccatttccttcaatttgagttaaaccaatgccatttgtatttacttcatacccagcgagacgtaaatcgttaataagagcttcgttcataattataccttagtaattttcaggtctgcaaattttttcttgcgttgatttttcatgcgacgaatagttttatcggaaatttcatgcttttgataagctttagattctacaccaaaagctttaacatcaaattctgacaagatatattgaaccaacaattcacggacagtattgcgtccaatcttctgatcattctgtttcatcgtttgatgaagctctttttcccatttatccaaaatttgaggagttacaatatcgcctttttctagcaaagaaactactttatcatatgcgtaagaattgatggcgtttttaatttgaatagtcatacattatcctcaattacgttaaaattttattatccaaaaaggccgaagcccttaggctaaactttttggcacccttccagccttcgtacatcattgcgactgacaatgacaaagctccttcacatgctgcatacttattattccagaaccaatttagaaaaacttcatcctcaataccgttgtgtttcatgttctggaaaaatttggcgcgttcttcagcaattcgctgcgataatttagattcaggattcatttaaatttcccaattgccattttcatcaataaatttaatccagtcatttactgaccacttggtcgtatcgccttttggagtaacatttaaagtgtatagcccttgcttaaaaagcatgcgtttgatattcatattttcctcagctgtaacgataacactcgtttgatttacgtttagcaactcgttgagaagtattataatcaaaatcatcatcaatgtaaactgattttttcaactttcttacttcaccgcgtaattgacgatttaactcatctttaacttctgaatcaatacctttcattctacgccatttatctgagcgaaaaatgttttcaaccatatctttatgacttaccccatcaggagctttacgctttccgaaatagtcataatcacgcacttttaagtctttacgacgatacgttttacccatggagtttaatttccttagcaactgaactaaatacagcacgatcacaaatcatacgtttatgtaacttgagtataagatagtaaacatcaaaaccatttacataagtaacacgaacaacattaccattcacgagataatactgtccctttttaatctctttatcaaccacaaccatatcaattcctcaaaggtaattcatatgttaataataccacggtttgaacttgttgtaaacaactttgtgaaaaatattttagggaatgataagaaaggaacgatagcttagaatggtaatatacagaatgtgagaaagaaaggcccagagggcccgtcttaatcttctatgatatctctatcatatccaagtgaaatgagagtttctttgaagtgtttaatgttcttttgtctagaatcattaatgaaaatgactggataacgaatgttaagagatgtgaatccagcgcgtttagcaagagatacaatcagcggacgatcatactcaatcttaccattatttgtaagaactttatagaaagtaaaaggagcattgagctcctttagaagttttgtaactgattgacatccaggacaacgacctacttcatctggaattccatagacttcaatcttattttgtttcacaactattccttactaagagcagcattcagcttgttagtaattttatccagacgctcattgaattcactagaagataagcccttttctggagaaatcaaactaatcacgaaaaatatagcaataaaaggaataaggaaaatagctccaactgccataaacagaaagaatgttacagttgtaagaaaatcagctaaacctttacgaaatttatacatttacatttacctttaattgattaaccaagcattgataagcactaaactatactgcgaataaaattctggaccaaaatgaaaatcatatcatttatagtatccataatgtaattcaatttaatcatgtttccacaccccatcggtatttgaccaaagtcgctgattatctgatcctcgccacagctttttggtcggaagatttttctcatacttcccatcaataataacatcaacatatttaagcatttctagttgtttaatatcttcaaacttatatcctgtccacaaccagatatctttctccggaaatcttgctttaacccaagaaactaaatttgaaatctcttctcggttctgtggataaagtgggtcaccgccggttaaggtaaggccttggatatacgatttgcttaaatgggacgcaagttctttaacggtattcatagtgaataactgtccgttacgagcattccaagtactacgattataacaaccttcgcatttatgcaaacatccagtgacgaagagaacgaccctacatccagggccgttaacgaaatcgcatggataaattctatcataattcatctttatatttccaagtatatcccttatgaatagattgaattcctttgcaacatttataaacagcagaatgaaaaaatcctgcatttcttatttcagttgctccagttagttctattgtatttccgtctggtgaatagccaataaccgttttggtgttaaagcgatttctagaactatttttgatatgctctttatgattagtagacaaagttcttcctgttagcccatcagatatcctttttccaaaatcatcaggaagagttactttagacctagctatgctcattagcttcttagttttatcgcttctttttgcgccacgctgattttcaaatttcttagcttttgcatacgcgtattgtgaagaagtaacttttctatgcttactattactcattatccaatatgcatcatacatctttccaccatatattttagccaataaatggtgagcagtatagtgggccttataagttaaaaatactaagttgtctaaatcatctgaaccacccatacttcttggaattatatgatgaagttcatacccggctttagtttgacggggcttagatgggtgttcagcagaattaactagatttgaatagattctgtcataattcattggtgcttaaccctatgcatgatttctttatttttaccgagattaaatccgcgttcgttcggatttcccaaataaccacatgttcttcttatggtattcatctttttaggatcagtttctccacaaatagaacaaacaaatccgttttcagtaggagtcatttcatgggtacttccacatgtaaaacatttatctactggcatattaacaccaaaataatctaaatgctgtgcagcataatcccacacggcctcaagaccttttaagttatttttcatatcaggaagttcaacataagaaatgtgaccacctgtcgcaatgaaatgatatggcgcttcacgagaaatcttttcaaacggagtaatattttcttctactgaaacatggaaactgttagtgtaccagcctttatcggtaacatcttttacgcttccatatttttctgtatcgagtttacagaagcgataacacaggttttcagcaggagtcgaatataaactaaaagcaaatccggttctttcagtccactgtttaagatgagcattcattttagttaaaatttctcgtccaatatcacgaccgacaagaatattcaattcgtgaataccaatgtatcctaaagacactgaacttctaccgtttttaaataactcaattatgtcgtcatcaggtttaagacgaaccccgaatgcaccttcttggtaaagaataggagcaacagtagctttaactccttttaaggaactaattctacacatcaaagcttcaaaacataaatccattcgttcattaaatagctcaacaaatttctgttcattgaactgtgttccaatataagaatctaacgcgatgcgaggaagattcagtgttacaacaccaagattattgcgtccatcaagaatttcattgccagtcgaatctttccatacgctcaagaaactacggcaacccatcggagaaacaggaacagatgaaccagtgatagctttattgttcttagctgaaataatatcaggatacatccttttgcttgcgcactctagagcaagctgtttaatatcatagttcggatcgtctttataaagattaacaccttcttcaacgaacataacaagcttagggaaaataggagttatcccatcacgaccaagacctttaatacgatttttcagaattgctttctgaatcattcgttcagtccagtcagttcccgtaccaaatgtaattgttacaaaaggagtctgtccgtttgaactgaataacgtgttcacttcatcaatgtgttcagtagagttcgctacttctctacccgtttatatgaaacaatgtaatttggataattgtcatctaaacacaaatctttaatacgacttgggtggagttttaaatctttagcagcatccacaaatgatttatagatattgtcatttattttaacatattctgggacgggatgaattgttatttttatatctataacatttggatgattactaacctgttcttcagagcatttaagaaattttgcagctgatttaaaacttctaaatttatttccggattttaaagctatagaaacagtctttttagccgttctactaccaatattatttttaacatgagcttcacttctactattatttttataatgctcaatcaatttctctcgtatcttatctttatgttttaaagataagattttacctttatgggcattactaagtttttgcttatgttcttctgaatccggatatttgttaaatttatatccacctatagatttattaagaataaattcgttattaaaatatttcctaataagcatttcttcatgtttaagggccgattcataagaatcaaaaacttgaagaattatccacttagctttataatctttaagcttttctttaacaagcttagatgacgaattgtattctttccaatttgtatctttaccatatatagttttgaattttttaaaacctatataaaaagacttatcaggaaatcttaccatataggtaaatgctacagaattggcaatttttaagctttttcttaatttccatttcatcgtttcacctcgtattcatatttatacgaggataaacagctgcatgtcaccatgcagttcagactatatcttcaactcttagagttgtctgccgtttcgggtcgcttgaccctactcccttacattcatcagggatagtcgttgggcatttacagctactgctgatttagcaacggattgtctcagtgagagtttcccgttttaggcagattttacatgagcttgacttacgttaactcataagcttggaatgcatcgtatacgtctttttctgttttagattgagcataattcaacgcatcagcgatttgccatttttctgcatcctcaatatgttttgcataggtgcgtttaacataaggagaaagtactttatctacattcgcaaaagtcgttccgccgtattggtgagaagcaacttgcgcagtaatttgtgccataattgcagtagcaactccaattgatttaggagtttcaatctgcgcattaccaagcttaaatccgttttcaagcattccttttaaatctactaaacagcaattagtaaatggaagagcaggggaataatcaatatcatgcacgtgaataattccgctttcatgcgctttcataataaaagacgggaccatatttttggcaatgtgtttagacacaataccagccataaggtcccgttgagttggaaaaacacgagaatctttattagcattctcgtttaaaaggtctttattagttttatgaattaatccttcaatttctttttcaattgtcattttaaactctttctaagctgcttcttgaatgaagctattaattgtgttttggtgtcagattcattatattcaaatcctctttgaagcatctcggccatcatttcctcttttcctaaacgagaaaattcctttgatttatctccaacaaagttagggtgaatattattttgggtgtaatcggattttaaataagtaagtaaattttctaaccattcaagataatcaacaccttgtccctttaagccagaacgattaaatttatgcttcatttgaccttctgcagcattgcatagattacaaagcaatccacgcacctttcctgcttttggtccatttaattcatggtcatggtcgaggtgattagcttgaacatcaggatttagttctcgttggcaaattaagcatttaccgttttgtgcatcataaaatttctgtttttcttctttgtataatttgccagtcaataacataataaacccttaccttaaatagataagggtatttattattttcaagtattgtaaaacatttgatgcaatcgcttatattgctgaatcattcggtcagaaaaagaaatttgagtttcaagccattcaatgtactctgcggcagcttgcattaaatttccttcatagccatcgttattttcttgtgcagctaatttagctaatgcgtatgaaatacgttcaccttgaaaatcggctttaggcttctgaacaacttgattagttcgctctacaacttcttcaatttcgccattttctactgattcagtattccacaaacaccaatacgtaattggcttatcgtagatgttaataatctttccatcagaaagttcaatttcaataattccagtgtcaggctctatatcatcttcacattcttttacaagttcacgaactttaaagacagtacctgcactaagttccggccagtaattacacagccctgtatcagcacgattaattctaaaccatttatctactgtaatcatgtcccatctccatatcaattaagtcatttatcgttggttcattatacaccgtttcttcatcagtgtaaaccggttcttccggctctggctctacagtttcccatctagccgcccaccaaggttttatagcgtaatccattctcgtactgtctgagttactacacgttctcgaagctcaaccagttcaacagtaggaactgcataataccagtcagaatggtaagaacctgagcgagattcatttacagccacatgtacattatgttttggactaaaataaacacactgacgatattggcatttaccatcttgcacccagtcttctatttcgataggtttaagatagtcggaaaaatcgaaatcatagttttccgaaaatgcatcatggtcttcaatgatttcgctcagaactgcatcaacatctaatttattttcaatattcatttttcacaccaccaactgcttacttcaaaatcgggttcgccatgttcccagaaccaggggccgataggaatccacttaccttcatcggtgacgtcatattcttcggatttaagcccaacgtatcgccattcgacttcataagcggtaccaccgattacaactgtgttctcgccatacgggttagacacaacaaagtctataccttctgctttagctaaccaaatcatgtattcatagagtttatattccaggtcgccttctgttccatcgcctagaaaaataatttgttcatttaattctatcatttaaagtattcccgcaattggtcaaatccaccaatatgacttccatcaggagcaaatacctgaggcattgttaagccgatttgagtatcacgacctagtttagtcagaagctcagcaattttctcatcatcaaaaacacctttttccggcataatgttgataaattcaaacggctgtttcttcacagtcaaaagacgttttgcattatcgcaatacacacatttatgaatgttgctatcataaccatatactttaaacatattattccttaattcctagtacttgtttaaaagtctcgtcgtaatcaagactttggcccgtttgttctttatgcttgtatataatatcacttacttctgatagcatatttttatatgaacgagttaaagcagatttaagcacgctgtatctatcaggaaatttaccgtgttcattataataagctattgctaattcacggactgccttttcagcagtctccatatattctttacgctttgtcattttcttctcggtcaaatcggtgtttacaatggcgacatttataacgaagattattagtctgccaatggaccaattgcacctgttcagttccacattcagggcaattaggaacgtttttagaagccctttcgcgacgttcaaccatgaccattacagaatcccaattaacaggagggctataatcatcacaaccataaatctttccacgcatttccagatcgtcttcttcaccagccataataattttaattagactagaattacttgaagctgcaatgtcttctaatagacgctttttcatttcaatacctcaatagcattacgtaaaccatttgctttggcggttaaatccttaagaactttagtatgctcttcaatctgtttttcaacttcaatcaaacgggtactgagatattcgcgttcttctttcatagtatcattcccatgattgggctttggcgtaggtttaaatttattagggtcctttaacttaataacaactttagtttcaaacagtgaaagaccataattcgtattatcatcaaaggattcaactacgtccattttagacaataacgtacgaagtttataagtcaaagaacgaatcgttttattatgtttattagcttcacatggtttcatatatggaagatttttatatatttcatgcggagcacaaacaataattaacaattcaaatggtccattctgagatgatggaatgaatttagccgtaacccaatcttttacatttacattaatagtacgatcatcgccatgacaaaacagtctacgagaatgcttaaaaagcatattcacattattattaaattcgtgtgaaaactcactggcaaatttttctttactatctttagaaaaccattcttctaaataagttcttttcacgtcgttaataatattaaatgtaacaacattatcatttgatacattagtctcaatgtagcttggatacataaattttttaacattattaacagctatcacagaagatagtaatcgggaacgtttgaaccactcgagcaaagaaccaagagaatgtgaatacgcattttgtgtatctttacaaatatggttttcccatccaatatagtctaaaaaatcacgaagaatattattgattacttctctatgtcctttctgataatcacgatgtttagtaataagactgtcaaaataatcaatataatgtttacgagttttcatgttcttctcacttggttaatgatttatactccgagccatccttggctttaaattttacttaattaactgcaaagcttgttctagacgatcaagacgattaacggattcttcccaaatctttttagcctgctcatattctttctgcgctttattagaaatttctagaacttctttataagctttttctagtgcaataacttctggacgaatttctatcggttcaggtgaatcatcaagacattccattagttcctcaagggtagtttcttctttaggagtattcacaatttcatcacatttttgttggtaaatttctttactagtaggtgaataagcacatttcacttcacgaaggctaattacgagtttatatccttcccaaccgcatatattcttaaaaggatatgtatgaatattttctactgtttccatacaaagtaatgcggtctctaattgacgagttatagtgctagcaattgagaaaaatttattaatattcttttggttagtttctggtgtaaaaaatccataattcacaaaaatagatactttatttttatcaagttcatattcttttatgataatcatatcagaagccaaaggatgaatttgacgataatcgccataacataatttagaagctgattttagaatttgcttcttgaaaagtctgaaattactaatccaacgacgagtaaaaatattctcagggtcttctttattattgagatgataagaattaacatcaccgaaccaattatatcctactacatctttagttcgtttaatttctttcccaaaattaccaagaatatcccgattaaccaaatatgaaagaagacgagactctttaaaggttttcttgataacatctttatctacacggctagaaattttacgaatttcatgaataaatttagtagaagaaacaatacttgcatcaacactattgagccactgattgataatagaaagaacgctattttggccaaacatcggtatagctttatcatcaataacgctattgaatgatttgatatattcgttacgagtcatattaatctcctcagtagaaagtaagaacattataccacatccttgtggcaaagtaaactagttcagtgcatttagtgcattgttcagtttagaacgttgctttgtcagattttgaacccttgactgagcttgttctaacgccttttcagcttctagcacttcattggtcgctttaactaattcagcatctactgccttaagagacttctcaatcgcatccgcgtgccatttttcaacaggtttaagacttggattttcaacaggaagaaaattcactttattgaatttccatgcatctttattacctgagctatacatccaaaatttaggatcgtttgataagtaattagataaatttaagttatcttgcacttcttgctttttctcttgtgtgacttcgtcctttctcaaaaattcatatttaaatgaaacgatcatatcaagttcatatgatgttgtatctaacttaaatcgttcaaaacgaggtaaaatcttagattgaacagcagcaacaacatccatatacttaaatgcttctgtcaactgcgatttaaggcattcgcaaattgaaagagaattttttgtattaggtttaaagctaattcgtgcagttctattattttcttttaacggtcttacttccatctgaagagtataaccatcaaatttcagattctttaagttaatgtcagagccttttaatcgactagcagtagacaaaatttgctttaattgtctacggaacagagcaataaatctccattcaatacgataatgatttggattgaaaaatccagatgataaatcgaccttactttgaccaacgccttgaacaaatagaggatttttataatcaatagttttacagaaatcggtgagctgttcacgagcagtcatttgagtaatataccctgctttactaaaattacgcacccattcactgctattcgtatacttaaagtgatgaataacacgattaactttatcagggtctaaattattttcacacaaaaatgtcataacatcacgagtatagctggcattacgaaccatatcttcaatttgagaacgagttttcatggtgttccttaagatttaagtaaatcaacaattttaattaacttttcacgctcagatttagctttactactcaatccagatagtctgaaaatttcatcatcatattgttgaatagaaatatcagctcttcaatttgcttattaaaataatcaatttgttcagaatgtttttcgttactacgaactggtacaggttttgtaggcaatttagttgaactggattcattcgggcgataaattaaaatgcaatttgaaccaatcgggcatttagcaccagatgaatattctaacgttccagcttcttttaaaacttcaaaagctaaacagagatgatgccccatattaacataatcagtagagcgagctctgatgtaataatcatctccacgagtgctaaatttaaagcatttaaggtcttctgtattattagttttaaaaatcatttgtttatctagacctttagccaatcgagcacctaatgctaataatcgtcgttgattttcccacaaatctgcgatcatttggtcaaatgataatttcggcattagccgaccataaaggtcatatcctttattataattgcgaaggatttcactcgcattaatacgagacaacgctccaattttattaagggctttcataatacgattagataaactcattccagacccgtttgttcgctgtaaatgtttttctaatccaaatccaatatcaactttaaatttatctaaaatttcagcatgaacatctctgtcaataacattcaaatccaaagttgggttaaatctatgaaaaaatttatctggctctccacgacgtaaaacagtccattcattcatcatttttttattaactaaagatttaattactgcgttgacattattaattactactgacatattttcctcactcaattttaccaattacgcggaataagattgaacagactatataagtaccacatatagattgaactaatgccattccaaagaaccaaacaatattatcaaaccatgtctgttttacgtcgaaaggacgtaaacttacagtaatattatcacctttttctattgaagaatacgtctctggggaaatatattcactaaatctataaccgtctttgagttcatatacagcaataaacgataaactagacccctttccttgagttcctgtaagggtattaactacagtaacatcataatctttataatgcatataatcattaattgcgtaataaccatatgcaattactatacataaacaacatatcaataaattcaatcttttaattatcaactgtttcataataatctcaattaaaagggcttagaaccattataccatccttggtataaagcggttatgcgagtaccgtatttaaccgttcttcaaacttccgaagagtgttctggcgttcagctctttgctttttgtaagtttcaatacgctctgaaatgagagtgtatcgttcatttactgattctttcataaaatcaggaatttctcgagaagctttaatctcgtcaaatttatcaataacagcttgctcttcagcaattaagttatcatacatcaaaatatctttcttgatgaactcaatatcttcttgagttacacgagataatttagatgctttatcctttttgtactgttcgttagtatcacgagaccagtgtaatgtacgatttttattcgtattcttgtaaatttctacaataccaatctcatcgataataacgatccaattccaacgggatttgtaaatttgtcctccattaacagtgatttcacctccgattgagatgtcattaaagaacttgctttgtgcttctgatttaaatttaccatcgttgtaatttaccaggttgaaaatatctttagcgttcattttgtgttcctccgtagttgatagttgtatagtaccacagaggaacggtcttgtaaacaactaaaagaaacttctttcacaattttttccactgaaccaagcgctcactgctttcttagtttcaggagcagtgttatccataaaccattcaaaggcagcctttttatgattctggagggcttctcgggctttaatctgctcacggtctattaacactaacatatgagcctttcttgtcaccaggggcttcttatgattttttgaatactcccaatcatttgtccatcgcatcgttgttgcgaattgaaatacagcttctttaatcttagtttcgtaaatttcacgagcctttgagtataacatcattacctccatttaccagtttaattctagtcatctttttgatggcagtccatataatctatttctgaactgcctttttgtcttagaaggcctcttatgaatttatttcagaagagtaacccgtagcgatttcttcccaaccgtttttgtcggtcataataaagtcagcaagataaagtgcagtacgcagtgaaacattacgtaaacggttaacattgactttcatccatgataatgctttataagtttcttcatcagaaagaccacgcttttgcatcatgtcggttgaaagaataacatcttcaaccctgaccataatttcttcattagtgtgaacacccaaatccaaataaactgagcgggacactaatgcttgtaaatgtggagcaagtttagtaccacggtctaattcgcggtcaatgtcaacgtttgtgataaaaacaattgttcctttaaattcgagctcacgctcaatgcctttttcttctaagtaagaagatgcagtgctccagcagactttacgggtctctccagtgtccagagcagctttcagaagattaagaatgtccatatcagagaaaacatccacatcatcaatcaaaaggacagaattctcttcacgattattccaaagctgttcataaagaccgataccagagattttaccgttaatgcttttatactcaatgtatccaatatcatttgctttattcaaagctttatctaaagaatatgttttaccaatacccgccgcaccagagataattaatgaacgaatatttccgttaataataccattcgtcatcattcccataacattaaatcttttattaatgcgggttttcatatcttcatatgattctttaacttcttcaacttttacaccatcatatgaaatgtctgatttgtaaacccaaacaccgcgacgtttaccgtcaatttcaacaaaaactttaccatctccttgtgcatctaccggagcattatcagggaaccattcacctaagagctcaaaagttccagagatttctttaccgaagtacatacccttattgatagttacagttttcattttattctccaaatccgtatcagttgatagttgtatagtaccataaagctttatgcttgtaaaccgttttgtgaaaaaattttgaaataaaaaagggagcccgaaggctccctatcatttataataacttcgatggttttcaagataaaccctctcaaggaagtcatcccagaaactcatgtctactttttgctgcataccgttcttagaagcttcagtagatgctgcttctacttgatcgaccacatcttccaaaaactcttgaacggttttaaatggatgcttacccaacttcacgtcgagaataaatggagcatcttggagtggataaaccaagtcaccagttttgtaaatttccaatagttgaagtccaccacgacaagcatggctcagagctttccagtcaatgccttcattggcttcggccttacgagcacgttcgccgtattcagcatctaatttgttcagtgactgcttaagctcaataagagaaagcgttgtctgatatttacgacccaacactgtgtaaaacgtttgtgggcctgttttctcatgattatggaacacccattcacagaattcgttttctggaagacgatgcttaatatcttcaactttagtacgacgctgcttaatggaaccatcttcttggtaatcaacccattgctcagggatttgattaactactttcaatacatcgcgtaatgcagccaaacgagaacccttgacgccgtatttagaagcttgcttgcggacatatcctaaatatgatttcatgttagtcgtataaaaacgagaacggttgtcttgaataaacttccamacatcaggcaaatcggatttaaccactagttcaggtggagtgtgaagcatatccaatgctacaggttcaccatctgctgctaatttaaagaaatacttaagactatacaattcgtggtcaatattatctttagtgtttttagatgatgtgttgttggtatttttactcatgtgctctttgacgtttccaataagaatatcgcgagcaggaggaacaaagatttctttaaaatctacatcagattctggagtagaagttccataaagatgactaccaaaataagacttaactacagttttcattattagacctttcataatcttcattaaattgtacaatcaatcgatgataattagattttggatatcctaatttacttatataagttccgaatgacccacgtttaggtctatttaatttaatccataacttataaagtaagtcatagtcttgccaatgtttaccagttctttgtctaattttagatgaatttgattgtttctttttagcttcaaaattattcaaagattttttgactgcagcagagtgtttttctttaactcccggtcgtttaaaagcagctttcacccctttagatattttttctttaacttccggtttgttttgtgcttctaactgggattctgaaattttagcccttatttcaggaagacttaatgttatcgaacgtttttctctgtattcattagacttccacatttctttcattgtattagaatgtatttttgaaaatttttctctaactaactgatatcctcttgaagttaatttgagatttcttcctaaagaatcttctccaaaattataaaatgaccaccatgcataaattaatccaggcgaattgtaatgaattttagctaaaagccaatgggctataaaatgctctctagctgttaataaaactagattatcagaatcatcattaccaccaatacaagatggaataatatgatgcttttccgtataaaaatttaatttagatttatctaattttctgagttttcctttcttaattaaattattatatactttagtataattcattggttcttcgtctcatttaattttgctttgcatttataacacatgtctgttccacttgaaataaacatatttcctcactttgaaatcatagttggaataacagaatcaagataagtctttagtgcaatagcttcctctttctttaatgtaatgatatgtgatcgaaaatcatcaatttgacgaatagatactacatctccctcttcatagcattttgaaacatttaaaatagtttcatcattctgattacaggagttagtaataatagcattacattttttaaaccattttaaattatgttttcttttagtagaatcataaaaatatttaatgttagttataaaattatcccaattattaattgataagcacattgactcgcttttaatattaaatcctgggcatgaagaataaaaatgaattttatgctcatcattaatgcttacaattttatcagtgtaagcatattcaatttgggttaaacgaacaatttcgctaggcgtaaaatataacatgtcatcttcttgcgtcaaacgatacatgttatttactttttctatagccaattcaccaaaaagtggactaacttctaatactagtcgtttcgcctcgctcatcattacatactcctctgaatcatattaataatgttattcaccagattataagtaaacattgggtaattatattgaatcatcacatatataacaaacaaaactttcattctcttctccttggcagttgacaagattactataccataatcttgtcaacttgtaaaccattaaatgacgttttcgataaaattttgaagctttgtatgagcatcaaccatgattttcatttcttccttggaaaagctgtgcctctttcccgagaagaacattcatagtcagaaactgcattagcatattcttcaattagcttcattaaaaacatctgtttttcagttttcattattccacctaatcatttcaagatattgaactaacttagctttggatttatccaaatcccttttagctgcttctataccgtcgtatgaatatccttcacaatgctcaactgctaattgatatgaatctatttcaatatcatgcgctaatttaatgattttttcaaactgttcgcgtgttagcatacttaaactctcgtattatgatcgataatttcatcaagaaacatatctaacgcttctacagcattattaactttagcttcaaattcttcaatacctgataaggcaaagttaatgcgttcctcatctgctttacgaattaaagctaccaattccttaattttatccgcttgttcgatactaatcattattccaccacatatgaaagagagaatattgcacacgccatgtgagttgcaacttcatcacacatattataacgtttcttaagaagttctacaagttcttcactagtaacttcatccatgtcgacgaaaaaatcaccattaatgatgacgtagatatttccttcttgattgagtgcttcaattttcatgatgttctcctctttatccgatggttgtatagtaccacagctcaaacggaaagtaaaccggtaaaatgaaaaaaagtctcccgaaggagactaatgttattcgagggaaagaagatacttactctggtaaaacattccagtaatatcatctatcgtgctttggatggctggaggcatttctttataaatgctgttagattggtctagtatgcgatcaatcattttaattgtgtcggtaggaagtttactggcatctggaattgaaggcgtgtattttcgaccagaataccccaaatattgctcaccaaatttatcaatcaaatctggcaactcggaaaaaataaaatcgtatgctttgtgtctagcataacttttagtttcaaaatgtgcagaatgaaaataagcttgtgcagccattaataaacctaagtattcatctgcctttgaaggttttccactttgtgaaaagtcgctgaatttcattcagtctccaatttaatgttcataattctagcgtatgattgtgccatctccgcgcctcgctctatacattcaaaatcagaagagcacgggtcatttttataggtcgttctcataaaactatagaattgttcagacgattctacgcttttattttcaaaaagcatataaacgtgcctaataccagattccataaatttatcaaaatgaggatcgacattcgcttcaatcgatggagataaaacaaatgacaatcctagcatggcaaaaagtgctgttgcttttaaggccataaaggcctcctatcatttttgtcctgtatttactttgtgccgatgcacggccttaactttatcaaggtatttttcaaaatttcgcaatctagtatagtctgccggagattggttgagtgatacttctcgacgcaaagctgaaatgatatttccaacttccctacgaatttcatctaattgaagaacagtaagattgcgaagttgcttttcagttaattgtagcatatatacccctttagttagataaacctatttataacttttgcactaaccgagctttttagttaattcattccaatgttttctacacaaagaaacataaatttcatcaccaatacaaatttgattaccttctttaactggtgttccatcttccattaatcgagctgtcataatcgcttttttaccacaatgacaaactgcttttagttcaataagtttatctgcaatcgctaaaagttctttagaaccttcaaataattttccagcgaaatcagtccttagcccataagccataacaggaacattatatgtatcaacaattcggctcaattgatgcacctgttcagtttttaaaaactgagcttcatctacaaatacgcaatgaatatctttttgtgcttcagcccatttatagaactcgaaaatatccatatcatctgtaataatattcgcttcctgcttaattccaatgcgagaaacgacttcacagacagaatcgcgagtatcaatagcaggcttaagaactaatacactcattccacgttctttataattatgtgcagcaatcaaaagagaagcagattttccagcattcattgctgcataagtaaaaattaaactcgccatcttagtccttagttaaattttctaaatatgtttctaaatcattttcagctttatcgatagattttactaattcgtaatatgtttcggcatctccatattcagaagatatttcaaaagacaaatccttttctaaactaataatttcaccaactaaaaataatatttcgttcttttgttcgcgagtaatcataaggaatttatataatcaatgagttcttgttctttattatcgaattctttagaaagttcttcgtactcgtttgcgctaaaaggaccgcattcattacaaactttttccaattcactatttttatccataacttcgtggataagagaaaagagtgtgtctttttgttctttgcttaaactcataaccatgtcacctttaagcagtattcttctacatgctgtttacgacctttcttatcaataaaggtatattcaacgaatgttccaatgtagtctttatctacatcatgtggactattaattggacatttagtgcgacaaatgcgttcccattgacgaataattactgccttattctttgggtcatatggatgtggataatgtatattcataataatggttcccaatcaacaatcacaatttctaatttagaggaatatgtatctaaaatcccctcaataatatcccagttccctttacctatgcctgcaccaatcctaggcatatagattgtaggtttaatcagtttattttcaccaaactcatttaattctaacatacaattcattaaagcggaatactcaaaatttggccctggttgaaattgagtataaagattgaagcagtaagctttatgagtcctaaagtatttttcatagactgagtaagaaccgagtttagttacatcaccccattcagtctgtaatttatcagcttccaaaattttagggaaagctttggttaattgacccgctacgcctgaacccatagtatgaaaacaattacatccatgtgcaatatttttaccttcagcgaaaagggcgacaatatcgcccttgatatattttacaatcatctagtactcaatcctcgattataagaatctaccaaacggtcaaccattgaatgacaagcggctttatctttctcctccgcaactgaacattctaaggtattccactttttagcatatcgttttaacaatgtatcgtttttgtatctgcttgatttatctctttctccgtctttatatgcatatattaatttctgtgcaaattcagcttggcatgccttatttttcccacaataatctgccgcagtgcggtttacatattctctaatttcagtatatgatgtatctgctgacgcagaagcagaaaatgaaattaatcctatacataaaaccaaaattttagtcatttactatttccaaaagtttattatttttaaggtaattagccttttctaggacttcagaagcatatttagaacctgctttaacattccatcccgaattataagaggatattgcttttcttatatcgcccttatgtatatttaaccaataagaaagttcaatgtacgcccaggaagctgaattggatcgtttattcaacattctttttatttcagcatcggtcatattataaccaagttccttaactcttgctcgcatagtaggcaaataattttggaacattccgtaggcgtgatgctttggtttagattttaaattaactccgccagagctttcttgccataaaatggcagccattatatgacctaatccgctcttgtggatatttttgtgtgttttatattttccatccttagaaaattgttccccgaattgatacgcgtaacgcatgttatcgagttggacattactgaaagtatgctcggagctatgtgccatcattgaaatggccaatagaccagcgagtagtgcttttctcatgcttacctcattgagttttaattactgctttagaagcctttcctggtaaacgacgactgttgataattgccatcctacattgaagtgacgggtctttgaacttctcgttaggtttacaaactgtaaatccaagccaaagatttccatctgtgatttctaaacgtccaggacgatattcaaccccatcaataaaatcctcgtcaatgtcaggacgcggaggcatactcaggaattcattaacttctaaaacatggtcttttattttatggaataattcaaaaacgtatgtctcatcaatctcccgttgaattgcgcgatcaagaagatgttgagaatattttagatgaaacgatgagactcctgctgcttttgatgcctcacgaatctcattgttaatttgacgaaactccgactcaaagtgacgacgaagcttatttcgacggataaaaacttctgtattgatagtcatgttattctcctcttaactgatagaaaaattataccacagtcaagaggaaaagtaaacagttattctttaaatctaatcaatttattcatagactttgaaacttcggcacgaacctcatgtagatttttgagctgttcaagacgctgcgtatagtaagcaatttcatcttcttcgagacagtcctgcgaatcttctttaagataacgtgcatagtcctggaaagcgttacggactacttcctggaagtcatcaagactttgaattttcttaggagcaacagatacacgacgaggggcagtataatactcataaccaaaccctgcgcttaattgagccattagtatttttcctctggttggaacactgcacgacaagcccacatactggcttctttgagtttcgttttagcaatagttaactgatcgagactttcagcataattcttcgcgaattcacagtcttcgcaattatctagtgcttcccagaattcatcatataaagcatcaaagataagtcctaaacgaacttcagcgtctttaatagcatttactttaccgattttctcttcagtatgtggtttataacccttaatatcttcaatcatatttgacttcctcaccagtacataatacgtattcaactaaacgaataggttcatgaatgccatagccttgaacagaaatttctgtcgtaggataaattccatcaatatcacccatattccacgcttcattaaattgctgttcgcctgagttactaaaccactcgcgaaagcatttagcacatcttcagaaccttcaataattatctttgccattacaaactttcagtaaaggtacgagcgataacgtcgcgctgctgttccggagtcagagagttaaagcgaactgcataaccggatacacggatggtcagctgcggatatttttccggatgcttaactgcatcttccagagtttcatgacgcagaacgttaacgttcaggtgttgaccaccttcaattttaactgtaggttgtggctcaatttcaatttcacgggcatgcaaaccatagaaaatttctgggtctacaaaagagtcctctttaaaggttttagagacaataattcgtgcttgaataccatcttcaaaataaatagtacctttatgtgtgccttcaagaatttgatatgctttcatataaacctcaattagaaaataaatttatccaagattgttctttaattaaaaatggctcagaatcatatgccattaaactttgcgtaattaatcctttaaaaggtccatcaataaattccatggtaaaatatggaattttattcattagccgtgcattaggagcagtgcacaaaactctgcatcctttgaatacgcctttttgtaatttgtattgcttgggataaaattcgctcaaaatgttattttttgccaaaatttcaaaatgattcaccaatttatttttaatagtttttggcgaaaaataaagatattcgaaaagctgagtgtctgtcatcattgcattccgattacgaaaaactgtggacgagtaataccaccaatgcaacatttactattacagcagtagtgtacggtgtcaatatggacactataaatcttatccatatcaggagatttgacaggctcatcaattatatacaaaattcgcgaaagctttaaacctctgaacttgcttcctttattaccaataaaactgcgtacagaatcagtaaataaacgaaaacgtatatcatcattagaataacgcgaaaattcctttttgatgttatttgcagaaattttagcataagctgaagtattagaaagaacaataactgttccgccatcatacaaccaattagcagcaaagttagtcacagcaattgatttaccggattgacgtccaccatctagtcgaagtgtacaatactgtttaagtaagtcttcaaatggcgggatatattcgtttttacaaatttcttctactctagcatcagaatggtgtgtaaaagcattcatcagggatagataaggaccagttaaaaatgttctcatttcttctctataagctctataagtttgggccattccgtggcacatgaattgtccatttctgtatttacccattaccgcgcttgggctcgaccttattacaggttggcgggaatccctcatataatcatgaggtccaggttgttcccttatgcataaatcgccttaccgtagtatttgtaccaagtaggacgttgtgcaattttttcatctaaacgagcttgtgatatagcaatagaagcttcatggggaatataatcaccacggaattcctgaggaatatcactaatatcctggactgtagtatccttgatattaaaaccacgttttaaacattcagctataagctcaatttgacgtttacgtaagaactcgagcttatcgtaaaagaatgtaacatgacctgcgccaaggataaaagtaggactgattttaaaatcacgaacacgtttaccgttagcaacatgcttacgaactgcaccaaaaacacgcggcaattcacgatattcagccattaagtgttggtcagccaattcagatactaaagtaaggttgatacgagtcattttagtgttctcctgtagttgataggtctatagtatcatacctacaggagatgtaaactgttatttatctttaattgctttagctgcttcgatagccgcttgctgaaggtcatccatagacatgccgaacttagaagcaaagttatcaattttcttttcgacggcattcaaaggcttggcctgtttaccttcattagcgccaggaagagcgagaatacgctctctgtcaatataaaggcttacaagtttcttacggtctttatcgggcaaatcatgaaatgaatgggcctttttatttacagcggcttctaatttacctgcgcccactcgcgcttcggcaataaattcttgatatgttttcatatgtttcctttaaatgtaaatatttttattattctatcctagaattgtgataatatattcacaattctaggagttgtaaactgcttttatttaagcgtcccaagtataagctttattaagaattaccacgggctgcattagcaacggcgtaagcgtactgaatattagcgtctttaaacttacctttagaggtatctatttctgccttaaagccgcctttagtcataacatcggcaaattctttacggaaagccattgcatcaagacctttccatgatgatttatgcttggcaaattcaagacctgcgaagttgacagctttagccaatttattatcaatgacccatttaccggctttaggcacaaacttcgggcctttctgtttagagaacagttttaaattccagcggcgaaggtctgcatcagctttaacaaattctgagtctaagttactagcaacaacatcttcaatatgagcaaatttaagtccgtcaacttcaatatttaaatcggtacgccagcgaagtccttcccaagcgaaggctttaaagtcggaagcctttgtagctaagtaccgttcaataggagctgttttagggtcaaagccgtttcctgatcggtaggtccactcatctttgttaatgcctttggcctttactacagaagcttcggcaataaattcttgatatgttttcatatgtttcctttaaatattttaattagtaattgtctattcaagtaattgtgaatatactatcacaattccaagagaaagtaaacagctttatagatttttatacgcgtcccaagtgccagttctaaacgttgtaatgactcgttttgcgcgattaggtgtttgattataccatatacttttagctaagttaactgctgcttcatcccagcgtttttgttgaagcatacgtaaagagttagtaaatcctgccacaccggtttctcccatttggaaaaccatattaatcaatgcacagcgacgaaccgcatcaagagaatcataaaccggttttaatttagcatttctcagaattccgcgaacagcagcatcaacatcctgattaaagagtttttcagcctcatcttttgtaattacaccattgcaattacgcccaatagctttatctaattcagatttagcagcattaagtgatggactttttgtaagcaaatgaccgatgccaatagtgtaatagccttctgtgtctttatagattttaagtctaagacgttcatctatacgtaacatttcaaatatattcataatacctcctaagtatttatagaaggtatttataaaattaaaagaggttgttcattattcggtaaagtgaaggacccatcacatattgccactgagtacgaggaataagagcaaaagcgtccatctctggaatcataacgccatctttattttcaaaataagactcgcaacggcaatttctgaacatctcatgctctactggaatcgtgtaataaaataactgtaggtctttattactagaatatttaaatacacctaggtcttctagaaggtctggattataattgctaaaaccagtctcttctaaacattctcttcgtgctgcatctaatgcgcttaaatcagaattttctacacggccctttggaatatcccaacgatgtgccatcattccagtcttacgagaaccagtaacccgacccataaataaatctttatcttctgtcataaagataataccagctgataatgttttcattttaatttcctgcattcagtgataaagttatttaaattttgagcatatttcttttcatcaaaaatcttttgctgtctgcgtaaccgccatggcatttcaatgaacatacgccatatccctagataataccgctgctgtaaaaatattaacaagtatagttaaaagaatccaatcgcctattctgtccattggatttttataaaaaagtaaaatacgaatgatgatataggaagactaatgatataccacagaagaaccttcttatctgtgaaccaatcagcattcgttaacttagcgcgaccattttgaatacacacgaatttatcatctgttacagtaaatggcttagctgcttgatatcccattctaaactccctaattaatcgtttctttgtatcttcggaacaaccattccaatcaactctatcaactggaatgccatcatccccatcatctaaatcataccagcgagtttttaaaatcatttaattttcctgcaatcaatcacaaactctttcattgattcattttcaatataagacatgtagctattatattcttttaattgtattttgtaatccttttttctttgccaatttattttaaaattatcataatgaaaatataaaatgataccaaagaatgaaaacaatgaaataattttagtataacaaagctcgctccaaacttctattatatctactgtaccactgatttttaaaataaaacagtcaattaatagtccaataagactacctgtaagagctgcagccaacgccacagcaaaaattaaaaatgactcagaaaacgaatatttgactttatttagctttggcttttgcatcgtgattccttaacaaatttcataatttcattaaattcatactcagcaagtttaagctggtgttcctttttaatctttttgcactgggctttccaatcacgtacgcgtttacgataatgtcttccttgataccagtatcctatccaatttacgggtactaataaaaatggaactaccaatggaagagttagcattaacataattattacgccagaatcaatatcagtcataacatctaaaacacctccaataatcaatagaattacaaatgatatagctatcacaggacctattaatacatcagtagaaattatctggcgctttaattcatacttcaaaggtttacttggaaggtatagtgatggctttgacatattctctacattccttaacaaatttttctagtaataaatcactttcaaaattaggattttccactaatttatcaaaaagatcatcaacaatattcaagatatttcttttactaagaatacgtttattttcatgcttcgtttcagaatcaactataagagtaaagaaatatttctttccctgaaattttaccgtagtatcaatataaaataaatttgacttttgtaaattacgtttaaaccatgcgtcacttaaactataaacaccgagataatcaaaatcgtcgtttaaataacaaactgaccattcaggagaaatgaaatcagtaaattcaacatcaaaatcacatgtcaatgaatgaattgattcaatactgttaataagtattccaggacgtattaaagactttttacctctggaaaatcttccagaaagactttcatcagtttcatatgaagaaccccaataataattacgtccttttgccatatgtttaagagcatttagtaattggtctggaacatcaacgtgtctttggaactcttcaaacattgaattgaaatcactttgcattttcattcctatttactccaagtaataggggccgaagccccttatcattatttcagagaattaatatattcctgaacatcggcagaggtagtttcaaccccagaaatattaccattaaaggtttcaactcgagcaagagtatcttcaatatcaaccttagtcagtgctgcaatttcaactacatcatcagcagtactaattccaagggcatttgctgcacgagtttcacggatatattccaatttaactgcaagttcttggcgagcatcatctaactcaactactttcttggcgatttcaattcgcatttcagcataaccatcagctttagtagtcagctgttcagctgttcgacgatatagcaagccgagtttagcatgcattgttacatcttgaccttcggaaagaagcttgcgaatttcacgctcttttgattcggcctgtttattcttttcaacaataagttcacgaatacgtttttcttcattaatagatttaacagaagcagtttttaggtctttaattttatcaagtagttttgctgctgcagcagtatactgttcttcaacagatagatttttagccattgcagaaccaagtttagtgcgaataaactcaacaattttcttcagtgtgttcatagtatttccttaggttggtataattagataatataatatcacgtttctaatagattgtaaacttattcttcgtctagctcgtcgataaaggcgttgatggcctcgataatggcatcattgatagccaataaaataaaatcatcgtcagtacctttagaagattctaaagcattgatatatgcttggttgacgagttcccaagcctttttaaaataaggagcttcatcatcaggacaaatatcccgcacgccttcaaagatacgtttggcataatctaacacccattgtacaggcatgttttgagaacgttcgttaaactctttaaagtccttagattcaaaaagctcttcaggataattatttctattacaaaaagctttactaaagttacgtttcataatgttttcctcatttgtataggctcataatatctcaatcatsagcctatgtaaacttatttcatattattgaaatattcttctgcgatttcgtcgttatcatggtaaactttagaagacagtttaacataactttcagcagtgaacatgttaatcacaacctttacagtataccactgaccgtcttcattacccattactgcgtaagtttcaaacatcggatggtcaggaccgataactttaatatcattcaccgtacgaccgaaatcttctgaaacacatttcataaagaagttgaaaagttcaccgtaattatccattttattctccaagttattttctgtatcagtagttgatagttgtatagtaccatggaagaacaaggatgtaaacagttttgtgaaaaaatttttaaaaagttttagggaattctagggcggagaggggcaattaaaagataggataatatattataaagggtataaactaaatgatgcctagagaggtctggaaaggcttagataccaaaaagccccaacctttcggtcggggctaaccgttgcggcaaccttgtcggggttccacctgccaaggcaagtgtttgtacgaaacgccgggattcgaacccggttattaagtagttgacgctactcaatatttttaaaaggccatatctcaaccatatccgaacgttccgtcaaaaacgctactcggcttacggcaaagatatttcctcgaatcgataatttggtgcgccgtttctgctgtgatgtaagagggcatcaataaacgcaaagattattaacgcaattccttactcagggaaccatcagtccgacgacttaccggtagcgacccggtttctcatttggtatcccgccctgggatcgaaccaggaccgcaaacttagaaggatcgtatgctatccattacaccagcgggacgtaatttaaaatttcatttttcgacctttaaaccatccttctggaattgggtcagttttcttaatacgtttagaaactttttcatctaatgaatgaatccacatcataccgaattgggaattcttttcacctttctggtgattatttttggcgtgagattctttcattttattaatagtttcaggagtatgatgcttatttagaaatctgctattatttaaaaatttttccctgtattcaggagttgaccacaaacgtttaaatacatttgaaccaattttacgatatttttcttgaagtaaaatatcattttcaaaacgtgacttaaacgatttagctccttttaagctagcatctttcttctggtttagcattccaggaatatttacatgatcccatccaccttcaccgccaagttttaaattatacacatctggtctatttaaaaactcttctgtgacaatatttttctcggcttcaagcatagattctttatcgtcaaaatactctaatatttctttagaaaaattttctataccatatttatcttgggctctttttaataatttaccagaacccatatatccatcatctaaattttcggtagaatgcacaccaatataaattttattattaattttatttgttattttataagtgtaatagaacataaatatctcctatttctaagagtatttatgttctcaaaatatgacccagaccagatttgaactggtaacctttcccttatgaggggactgctgctaaccattgagctacagggccttggtgctgattgacggaatcgaaccgccgacatcctcattacaagtgaggtgctctacctactgagctaaatcagcaaaattacggaggcgataggatttgaacctatgagtcgccggagcgactgccggttttcaagaccggtgcattaaaccactctgccacgcctccagtctccatacaaggatttgaaccttggacctcctgatcccaaatcaggcgctctaccaaactgagctacacggagtaaattaaattggagcggataatgagaatcgaactcacatcatcagattggaagtctgaggtaataccattatacgatatccgcaaatttggtgcgagaagtgggactcgaacccacaaggaaatcattccgcagcattttaagtgctgtgcctttaccaatttgaccattctcgcgctgggaataaaggactcgaacctttgcatctagcagtcaaagtgctatgccttaccaacttggctaattcccaattattaacaaaggctctctaacaagaacccttgatgatagagggtattaatcagtgcggtatgagttaataataacaaataattcttaaagcatatttaccatttatgatgatacgtatttacgatacattcaagacccaaaggattcttgaaaatatcatattcaagaggacctttttctgtttcaataaagaaatcaaaatttactgtattaaatttacggtcttcctttactaatttaacttgagaagatgaacgatcaatgtaaaccttttcaacttcaaaacacgttaaaatgccataatcatcaatcaaggctttagctgcttcttgatcatatttatatccattttcaacggatgatactttcgcataaagaatcatcatcaacctctatcaacaatagcatgagtatgggcatttacgatttgccaccagtcgaaacgattggaaccataatctggtttattttcattttctttaatgatatcacgcagtttatcttctgtttcagcatacgcaattaaatcatcatatccaccacaaggataataattatcacctgcgaataaaagaaaatttaccttagatggatttacgtaataatggtctttaggatatttagttcctctccaatcagttacttcaacataacggtaagaaaatccatttttactttcaatccaactccacgcttcaaaaggagtattaaaaactttatcaggtattaaattaccttcaaaatgagaaggatttgcataatccccggcataaacataatattcgttaatactcatttattcacctttagaaattttatccataacgatagcaattaaaccaattaaaaatgctactacaagtgaaaacacattttctgctgtagttaataatccgcatataaatccaacaaacattgaaaaactaaaagcagaagcagaaattgcaatagcaacatttcgaattaattcacagcgtttcattttattctcctcagtagttgatagggtaatagtatcacagctaaaaccctatgtaaacaactttgtgaaatatttattacaaaagatttttagcaataatcttgagatgtgccgcagaaatgtgtttagctttaaacaacgcagtttcttcagcaggagagataacgattgtagcaccatcctttttagcagaccacccatcacctaggtaaacagtacctttgatttcttcgccatcaaccagactaatcattggtttaccttctcgtcctttatttgctttaataacttcagaagtaagagtagcttcggtaatggtagaaaccggggtagttgtagaagtaaattctttaaatgttttcatttttattttcctaattaattttgatgaggtaatagtatcactacctcatcagtatgtaaacaactttgtgaaattattttaaatcatctgcccaatcgagtttaagaggctctttgtattcacgatctaatacgaccggaatttgtacatcaccgctaaatgataagggcccaacattataagacaatgttatatgcggtgtgtaatcatcaaaatcatgtgtagcacctagtgcccgcgcatacatgtgtcgacagcgcagatattcagaatctagcacaagtacaagagtcgatccatcttgtgttttccatacttctaaatgtccagaagaagctacttcaaaacttccactcgatggaacatatggaacatttactcttgaataacatatagtcgaatgaattttttctctaggaactggattaggaacacgtaaagagcgctgaagttcttccagcgcatcaagtgttaattctgaaaacttagctgctacataaagacccgttgaaaagtctttaaattccatcattcttcatcttttgcttcatctgcagattcagcagtaagatttttgacagcttcaacgatttcttcaactttgatagtatcgccagtgatacctactgcacgagcaatttcagccaaagttccttgcagaattttggattcttccatcagacgagcagcttgatcctgcgtatcaagaatgcgagatttcagagttacgatttcagcagacagtttttgttcaacagtttgttcagacattatagtacctttagtgtatttttaattttagaaaaaagttcttcaagagaaccatcgtttgtaattactaaatcgccatcacgaattggcaatccagcttctgtaatatgtgtatcattggatttttgaccaggacgaactacatgaattactgtagcacccatcgccctagccgcatccatttcatgatcttgacgggtatcaggaacgatataataatcataacctgagttaaatttatcaagataatctaaagcaaataattttacccagtacatgcggtcgaagttattaacaatcaaatccgtacctagggcttgcatcagacgacggactgaccattgatcttcaatattatttataacgtcagtaatcttattaaatgctacgaaattaactgattcttttccttcgtcatcaaaaacaaacacacctttaattgggcttttaccattaagataacaaaatgcttgttccataatcgtgattacttctaatttagtcagatttaaattagtctcacgatcatagtcaattccttcaaactctttacgagttaagcaaggatagtcagtgtttgctgcaaatactccccatgcataagccaatgcatccttaataggaccagcaagttggtatttaactgcagaataattgctcatgataaaatcagcagtagtatcttttccactacgctttacaccgcttaaaaagattagtttcatgtgtttctcctcaaatttaattaagattataacacacaaaactgaagcattaaacttctgctataattttaccatctttttctacttgaaaataggtgtaaggaattgttgcagtacatactaaagccgggtctgaatcttccgtgtagctaaattctacttcagataggtcagaaacccaaggcttataaaaatttattgacatcacgatttcagttttgctattatctaagatgtaaagcgtaatgtactcaggacctgttttttgggcagtattttcacctgtaagatagttgctagttcctagcatccattcatacattcctatccacgacttaagttcttcatcaactataaatctcacaatgagtggatcatactcaaatgtaacacctggacgttgtgctcggcccagtccaaacggcccagtcacggtatcagtaacaggtattctaattcctggaataggaactgactgagcatttaaagtaaaagcagatgtagtattactatgtggtattgatactacaaagttagttgtatttgcttggttaaaaatttgttgcagagcttgcgacatatattcctcataatgctttataactgttggtggtataatgggtctaagtcccttccattcaattccatttagaacaaacaacagaaaagaatggaagataatagaattagatatttgaccagactttgtttgcagagaaacgttttccttttgaaacgaactgctgaagtggcatcaacacaacgttcgcccagtctttcggggcgatttcaacaaggctacccataatattaccaggtatatatgccttaatcatttggtctgcacccctaaatcctttcacttgactccaatcaatttttaatttcgttttattagtaatagtaggtgtatttgcatattgctttaaaagctcttctaggaattgctgacgagctttaggtggaatatagtgcaagtttaatccgtacattaaattatgcttacctaaaccaaggtaaattatcaaaggaaatttatcccagtaaggaagagtttccttgtgtttagcatcataagcaaaagcatatattcgtcccggctgcgggcgaacaactttatgtccttttacttgcttaatagtttcagcaaaccactttctggttttattattaattgctgcgccttcattacgaattttatcacgcaatgtttgtctgaatgaatttatcataagcagttgtctttcttgcttattgagtttattcattggttttgattcaagtttttgaatcttttcagccgttttaattcctgaagcatattttgacattgctgaagtaaacgtagagtatttgattcctctttcttcagcaaattgctttcctgtcattccttttgctttggcctttttatattcaagacctatctgaatccatttcttttcgtttaatgattgcttaacctttggaacttggggagtgctttcattaattatttgaaaaatagccattatgcccccttaaagccaagagctcgtaatccatcttctgttagaattctaaattttattccacgcttttcagctaaagattgtgctgctttccatttgtcagtgttcacagaccaggtataaatttcattcataaatcttttcttcgctgcggtcgttagatgtgctggtttaactggtggttgtgtttcttttttaggttttatttcaataaaaaattcttgtccagaagaatctttcatccaaatatccatgaagtatctacgttttttcccttctgcattacaaaaataaggaattactgctgtttcactaccccatgcaataatttctggatttttatctaaccattcaaaaaagaatttttcccaatttgatctatacgtaatttttttagggtcacctctatactttgatatatttttaggaacccattttccagaatatgccattggattctccttataaatagataatatatttataaacaggagggcccatgctctttacattttttgatccgattgaatatgcggccaaaacggtgaataaaaacgcgccgactattcctatgacagatatttttagaaactataaagactattttaaacgcgctcttgcgggataccgcttacgtacttattatattaaaggttcaccacgcccggaagaattagcaaatgctatatatggaaatccacagctgtattgggttttattgatgtgtaatgataattatgacccgtattatggatggattacttcgcaagaagctgcttatcaagcatctatacaaaaatacaaaaacgtaggtggagaccaaatagtatatcatgtgaatgagaacggtgaaaaattttataatttaatatcatacgatgataatccatatgtttggtatgataaaggcgataaagctagaaaatatcctcaatatgaaggagcgcttgctgcggtcgatacgtatgaagctgctgttcttgaaaatgaaaaacttcgtcaaataaaaataatagcaaaatcagacatcaattcatttatgaacgaccttatacgtataatggagaaatcttatggaaatgataagtaataaccttaattggtttgtcggtgttgttgaagatagaatggacccattaaaattaggtcgtgttcgtgttcgtgtggttggtctgcatccacctcaaagagcacaaggtgatgtaatgggtattccaactgaaaaattaccatggatgtcagttattcaacctataacttctgcagcaatgtctggaattggaggttctgttactggaccagtagaaggaactagagtttatggtcattttttagacaaatggaaaactaatggaattgtccttggcacgtatggtggaatagttcgcgaaaaaccgaatagacttgaaggattttctgacccaactgggcagtatcctagacgtttaggaaatgatactaacgtactaaaccaaggtggagaagtaggatatgattcgtcttctaacgttatccaagatagtaacttagacaccgcaataaatcccgatgatagaccgctatcagagattccgaccgatgataatccaaatatgtcaatggctgaaatgcttcgccgtgatgaaggattaagattaaaagtttattgggataccgaaggatatccgacaattggtattggtcatcttatcatgaagcagccagttcgtgatatggctcaaattaataaagttttatcaaaacaagttggtcgtgaaattacaggaaatccaggttctattacaatggaagaggcgacgactttatttgagcgtgatttggctgatatgcaacgggacattaaatcacattctaaagtaggaccagtctggcaagctgtcaaccgttctcgtcaaatggcgttagaaaatatggcatttcagatgggtgttggtggtgtagctaaatttaacacaatgttaactgctatgttagcaggagattgggaaaaagcgtataaagccggtcgtgattcattgtggtatcaacaaacaaaaggccgtgcatcccgtgttaccatgattattcttacggggaatttggaatcatatggtgttgaagtgaaaaccccagctaggtctctatcagcaatggctgctactgtagctaaatcttctgaccctgctgaccctcctattccaaatgactcgagaattttattcaaagaaccagtttcttcatataaaggtgaatatccttatgtgcatacaatggaaactgaaagcggacatattcaggaatttgatgatacccctgggcaagaacgatatagattagttcatccaactggaacttatgaagaagtatcaccatcaggaagaagaacaagaaaaactgttgataatttgtatgatataaccaatgctgatggtaattttttggtagccggtgataaaaagactaacgtcggtggttcagaaatttattataacatggataatcgtttacatcaaatcgatggaagcaatacaatatttgtacgtggagacgaaacgaaaactgttgaaggtaatggaactatcctagttaaaggtaatgttactattatagttgaaggtaatgctgacattacagttaaaggagatgctaccactttagttgaaggaaatcaaactaacacagtaaatggaaatctttcttggaaagttgccgggacagttgattgggatgtcggtggtgattggacagaaaaaatggcatctatgagttctatttcatctggtcaatacacaattgatggatcgaggattgacattggctaatatacttccaatgagcgctgatttaggagaatccatggaaggttcttctatcgacgtcacctttaccgctcaattagaaacaggtgaaacgttagtatctataaatataactagttacgaagaaactcctggggttttagtagaagaaaatcgcttatatggaacatatgaatctgtatttggtttcggaaatgacgcgttgaaatatcgtttaggcgatgaatttaaaactgctgcttcatgggaagaacttcctactgattctgatactcagttgtatttgtggaaagctcctcaaaacctccagaagacattcacttacgaagtaacattaatatatgactaccaagaacaaagtgaatctgggggttctggcagtaattctaggtcatcttctgatactactgaaccgacagatcctcctgctccagtaagaaaaactctagttaaaaattatactaaaactatagttggaaattggagtcgttgggctaataaactgagaaaatatgcctatgcaagaccataaatatttttatttgtattcaataactaataaaacaacagaaaaaatttatgtaggcgtccacaaaacttcaaatttggatgatgggtatatgggttctggcgttgccattaaaaatgccattaaaaaatatggcatagataatttttataagcatattataaaattctttgaatctgaaaaagctatgtatgacgcagaggcagaaatagtcacagaggaatttgttaaatctaagaaaacttataatatgaaactaggcggtatcggtggcttcccaaaacataacacagcgggtgctaaaaatggattttacggtaaatctcattcgcgtgaaactagattgaaaattagcattaaatcgtctagaaaaagagggcctagagggctagaggtaaaactctgaagatgtgtggcgccaataacccaaggtatggcaaaatagcccctaatgctaaatctgttattatcaacggcgttttatataaaagtattaaaatcgcagctaaagctcttaatataaattatagtaccttaaaggggcgagttaaagcggggtattataaatgtcaggattaagttatgataagtgtgttactgctggccatgaagcgtggcctccaacagttgtgaatgctacacaaagtaaagtattcactggaggaattgctgttctcgtagcaggcgatccaattacagaacatacagaaattaaaaagccgtatgaaacacatggcggagtgacacaacctagaacttctaaggtatatgtcactggaaagaaagctgttcaaatggctgatccaatatcatgcggtgatactgtggctcaggcatcatctaaagtattcattaaataggatttaaaatggcaaatacccctgtaaattatcaattaacaagaacagcaaatgctattcccgagatattcgtcgggggtacatttgctgaaataaaacaaaacctcattgaatggcttaatggccaaaatgaatttttggattatgattttgaaggctcaagattaaacgttctgtgtgaccttttagcttataatacattatacattcagcagtttggtaatgctgctgtgtatgaaagctttatgcgtactgctaacttacgaagttcagttgttcaagctgcacaagataacggatatttacctacttcaaaatccgctgcgcagaccgaaattatgttaacatgcactgacgcattgaataggaattacattactattcctcgcggaactcgctttttagcatatgcaaaagatacttctgttaatccatataacttcgtttctagggaagacgttattgctattcgtgataaaaataaccaatattttccgcgtttaaaattggcccagggacgtatagtaagaactgaaatcatttatgataaattaacacctattatcatttatgataaaaatattgatagaaaccaggttaaattatacgttgatggagcggaatggattaactggacgagaaagtcaatggttcatgctggttcaacatcaacgatttactatatgcgtgaaactattgatggaaacactgaattctattttggtgaaggtgaaatttctgttaatgcttctgaaggagctttgaccgctaattatatcggaggtcttaaacctactcagaactctacgattgttattgagtacattagtactaatggtgctgacgcgaacggagcagtcggattttcatacgcagatacattaacaaatataactgtcatcaatattaatgaaaatccaaacgatgatccagattttgttggggcagatggaggcggtgatccagaagatattgagcgtattcgcgaattgggtactattaaacgcgaaacccaacaacgatgcgtaactgcgactgactatgatacattcgtttcagagagatttggttctattattcaagctgttcagactttcactgattctactaaacctgggtatgcatttattgctgctaaacctaaatcaggattgtatttaactaccgtacagcgtgaagatattaaaaattatctcaaagactataatttagctcctattacgccatcaattatttctcctaattatctttttattaagactaatttaaaagtcacatatgctttaaataaactgcaagaatccgaacagtggcttgaaggtcaaataattgataaaatagatcgctattataccgaagatgtagaaatttttaactcgtctttcgctaaatctaagatgttgacatatgtagatgatgcagatcattctgtcattggttcatcagcgactattcaaatggttcgtgaagtacaaaacttctataaaacgcctgaagcgggtattaaatacaataatcaaataaaagatcgttctatggaatctaatacgttttcatttaattctggacgaaaggttgtaaatcctgatactggtttagaagaagatgtattatatgacgttcgtatagtatcaacagaccgagattctaaaggaattggtaaagttattattggtccatttgcttctggcgatgttacagaaaatgaaaacattcagccgtatacaggcaacgattttaacaaattagcaaattctgatggacgcgacaaatactatgttatcggtgaaataaattatccagctgatgtgatttattggaatatcgctaaaattaatttaacatctgaaaaatttgaagttcagaccattgaattatattctgacccaaccgatgatgttatctttactcgcgatggttcactgattgtatttgaaaatgacttacgtccacaatacttaactatcgatttggagcctatatcacaatgacagtaaaagcaccttcagtcactagtctcagaatttccaagttatccgcaaatcaggtgcaagtacgctgggatgacgttggtgctaatttctactattttgtagaaatcgctgagacaaaaacaaactcgggggaaaatctcccgagtaatcaatatcgttggattaatttaggatatacagcaaataatagtttcttttttgatgatgctgatccattaacaacatacattattagagtagccacagctgcgcaagattttgagcagtctgattggatttataccgaagagtttgaaacttttgctacaaatgcttatacatttcaaaacatgattgaaatgcaattagccaataaattcattcaggaaaaatttactcttaataattctgattatgttaattttaataatgatactataatggctgcattgatgaatgaatcattccaattcagcccatcgtatgttgatgtttcatcaataagtaattttattattggtgaaaatgagtatcatgaaatacaaggttctattcagcaagtatgtaaggatattaaccgagtttatttgatggaatcagaaggaattctatatctttttgagcgctatcaacctgtagttaaagtatccaatgataaaggacaaacctggaaagctgtaaagctcttcaatgaccgtgtaggatatcctttatctaagacagtatattaccaatctgcgaacacaacatacgttctaggatacgacaagattttctatggccgcaaatctactgatgttagatggtcagccgatgatgtcagatttagttctcaggatataacatttgctaaacttggcgaccaattacatctaggatttgatgtagaaatttttgccacttacgcgactttaccagcgaatgtataccgcattgcagaagctattacttgcaccgatgattacatttacgttgtcgccagagacaaagttagatacataaaaacgagtaatgcacttatagattttgatccattatctccaacatattcggaaagactttttgaacctgataccatgactataaccggaaatcctaaagcagtatgctataaaatggattctatctgtgataaagtttttgctcttattattggtgaagttgaaacattaaatgctaatcctagaacatcaaaaataattgattccgctgataaaggaatatatgttttaaatcatgacgaaaaaacatggaaaagagtttttggtaataccgaagaagaaagaagacgtattcaacccggatatgcgaatatgtcaactgacggtaaattagtttctctgtcttcgagtaattttaaatttttaagtgataatgttgttaatgaccctgaaactgcagcaaaatatcagttaattggcgctgttaaatatgaatttcctcgtgaatggttagctgataagcattatcatatgatggcatttatagcggatgaaacatctgattgggagacttttactcctcaaccaatgaaatactacgcagaaccattctttaactggtctaaaaaatctaacacacgttgttggataaacaactctgatagagctgtggtagtttatgctgatttaaaatacactaaagttatagaaaatattccggaaacatcaccagatagattagttcatgaatactgggatgatggtgattgcactatagtaatgccaaatgtcaaattcactggatttaaaaaatacgcatcaggaatgcttttctataaagcctccggtgaaataatttcttactatgattttaactatcgtgtgagagatacagtagaaattatttggaagccaactgaagtatttttaaaagcatttttacaaaaccaagagcatgagactccttggtcaccagaagaagagcgtggattagctgaccctgatttaagaccattaattggcacaatgatgcctgattcttatttgttacaggattcgaattttgaggcattttgcgaagcatatattcagtatctttctgatggatatggaactcaatacaataatttacgaaatttaattcgtaaccaatatccacgagaagagcacgcatgggaatatttgtggtcagagatatataaaagaaacatttatttaaatgctgataaacgcgatgctgttgcgagattctttgaatcacgtagctatgatttttattctactaaaggaattgaagcatcatacaagtttctttttaaagttctttataatgaagaagttgaaattgaaattgaatctggggctggtactgaatatgatataatcgttcaatctgattctttgactgaagatttagtaggacaaacgatttatacggcaacaggaagatgtaatgttacttatatagaaagaagctattctaatggtaaattgcaatggaccgtaactattcataatcttttgggacgattaattgctggtcaagaagttaaagcagaaagactccctagttttgaaggcgaaattattcgtggggttaaaggaaaggatttgcttcaaaacaatatagactatattaatagaagtagatcatactatgtaatgaaaattaaatccaatttaccttcttcccgctggaaatctgacgttattcgttttgttcatccagtaggatttggatttatagcaattacccttttaacaatgtttattaatgttggtttaactcttaaacatacagagactataattaataaatacaaaaactataaatgggattctggattgcctactgaatatgccgacagaatagctaaattaactccaaccggtgaaattgagcatgattcagtaacaggcgaagcaatttatgagcctggcccaatggctggtgtaaaatatcctcttcctgatgactataatgctgaaaataataattcaatatttcaaggtcaattgccgtctgaacgacgtaaattaatgagtcctttatttgatgcatctggaacaacatttgcgcaatttagagatttagttaataaacgtctaaaagataatataggaaatccaagagaccctgaaaatccaacacaggttaaaatagatgaatgattcaagtgttatctatcgtgcgatagttacttcaaaatttagaacagaaaaaatgttgaatttttataattcaattggaagtggtccggataaaaacactatctttatcacatttggaagatcagaaccgtggtcatcaaatgaaaatgaggtgggctttgccccaccttatccaaccgattctgtattaggcgtaactgacatgtggacgcatatgatgggaacagtaaaagttcttccatcaatgcttgatgctgttattcctcgcagagattggggagatactagatatccggatccatacacatttagaattaacgatattgtagtgtgtaactcagctccttacaacgctactgaatcaggcgctggttggttagtgtatcgttgtttagatgttcctgataccggaatgtgttcaatagcatctttaactgataaagatgaatgccttaagttaggtggaaaatggactccttctgctaggtcaatgactccgcctgaaggtcgaggagatgctgaaggaacaattgaacccggagacgggtatgtgtgggaatatctatttgagattccgcctgatgtatctataaatagatgcacgaatgaatatatcgtggttccttggcctgaggaattaaaagaagacccgactagatggggatatgaagataatctcacttggcaacaagatgattttggattaatttaccgtgttaaggcaaatactatccgttttaaagcatatttagattcagtttattttcctgaagctgcattaccaggaaataaaggatttagacaaatatcaataatcacgaatcctcttgaagctaaagctcatccaaatgacccaaacgttaaagctgaaaaggattattatgacccagaagatttaatgaggcattcgggtgaaatgatttatatggaaaataggccacctattattatggcaatggatcaaacagaagaaatcaatattctgtttacattttaaattaagggagcccatgggctccctttttctttataaatactataaactcataaggaaaccgctatgttcattcaagaaccaaagaaattgattgataccggcgaaattggtaacgcttctactggtgatatcttattcgacggtggtaataaaattaatagtgattttaacgcaatttataatgcgtttggcgatcagcgtaaaatggcagtagcaaatggcactggagcagatggtcaaattatccatgctactggatattatcaaaaacactctattacagagtacgcaactccagtgaaagttggcactagacatgatattgatacctctactgtaggtgttaaagttatcattgaaagaggcgaactcggcgattgtgttgaattcattaactctaatggatcaatatcagttactaatcctttgacaattcaagctattgattcaattaaaggtgtttcaggtaatttagtagtaactagcccatatagtaaagttactttacgctgtatttcatctgataattctacgtcggtttggaattattctattgaaagtatgtttggacaaaaggaatcaccagctgaaggtacatggaatatttctacatctggatcagttgacattccattatttcatcgtactgaatacaatatggctaaattgctagttacgtgccaatcggtagatggaagaaaaattaaaacagcagaaataaatattcttgtggatactgttaattcagaggtaatttcttctgaatatgctgtcatgcgagttgggaatgaaaccgaagaagacgaaatcgctaatattgcatttagtattaaagaaaattatgtaacggcgactataagttcttcaactgtcggtatgagagcagcagttaaagttatcgctacgcagaaaatcggggtggctcaataatgaaacaaaatattaatatcggtaatgttgtagatgatggtaccggtgactacctgcgtaaaggtggtataaaaataaatgaaaactttgatgagctttattatgaactcggtgatggtgatgttccatattcagccggtgcctggaaaacttataatgcttcatcaggacaaacattaacagcagaatggggaaaatcatacgctattaatacatcttctggaagagtgactataaatcttccaaagggtacagttaatgattacaacaaggtaattagagctagagacgtatttgctacatggaacgtcaacccagttacactagtagctgcttccggcgatacgattaaagggtctgcagtaccagttgaaattaatgttcgattcagcgatttagaactagtgtattgtgccccaggacgttgggaatatgtcaaaaataaacaaattgacaaaattaccagttcagacattagtaatgtagctcgcaaagaatttttagttgaagttcaaggacaaacagactttttagatgttttccgtggaactagttataatgtaaataacatcagagtaaaacatcgtggtaacgaattgtattacggcgatgtgtttagcgaaaacagcgattttggctctccaggcgaaaatgaaggagaactggttcctcttgatggatttaacattcgattaagacagccttgtaatattggtgacactgttcaaattgaaacatttatggatggtgtatcacagtggagaagttcatatacaagacgtcaaattagattgttagattcaaaattaacgtcaaaaacttctttagaaggaagcatttacgttactgatttatcaacaatgaaatcaattccattttctgcttttggattaattccaggagaacctattaatcctaactctcttgaggttcgttttaacgggattttacaggaattggctggcacagttggaatgccattatttcattgtgttggtgccgattcagacgatgaagtagaatgctctgttttaggtggaacttgggaacaatctcataccgattattcagttgaaactgatgaaaacggcataccagaaattttacatttcgatagcgtatttgagcatggtgacattatcaatatcacctggtttaataatgatttgggtacattattaacaaaagatgagattattgatgaaactgataatctctatgtatcgcaaggacctggagtagatatttctggtgatgtaaatttaacagacttcgataaaattggttggccaaatgtagaagcagttcaatcttatcaacgcgcatttaatgctgtttcaaatatctttgatacgatttatcctattggaactatatatgaaaacgctgttaatccaaataaccctgttacatatatgggattcggctcatggaaattatttgggcaaggaaaagttttagttggatggaatgaagatatttcggaccctaactttgctctaaataacaacgatttagattcgggtggaaatccttcacataccgcaggtggaacaggtggttctacttctgttacattggaaaatgctaatcttcctgcaactgaaacagatgaagaagttctaatagttgatgaaaatggatcagtcattgttggtgggtgtcaatacgatccagatgaatccggtccaatttacactaaataccgtgaagctaaagcatctactaactctactcacactccgccaacatcaataactaacattcaaccatatattacagtttatcgttggataaggattgcataatgagtttacttaataataaagcgggagttatttcccgcttagccgattttcttggttttagacctaaaactggcgacattgatgtaatgaatcgtcaatcagtcgggtcagtgacaatatctcaattagcgaaaggattttatgaaccaaacatagaatcagctattaatgacgttcataatttttctataaaagacgttggcacaattattactaataaaactggtgtttctcctgagggtgtttctcaaactgattattgggcattttctggaactgtaacagacgattctcttcctccgggttctcctattacggtattagtatttggtcttccagtttcagcaacaactggaatgacggcaattgagtttgttgcaaaagttcgcgttgcactacaagaagctattgcgtcatttactgctatcaattcatataaagaccatccaactgatggtagtaaattagaagttacttatttagataatcaaaaacatgtattaagcacatattctacatatggaataactatttcccaagaaattatatctgagtctaagcctggctatggtacatggaatttattgggcgcacaaactgtaactttagataatcagcagactcctacagtattttatcattttgagagaacagcatgagtaataatacatatcaacacgtttctaatgaatctcgttatgtaaaatttgatcctaccgatacgaattttccaccggagattactgatgttcacgctgctatagcagccatttctcctgctggagtaaatggagttcctgatgcatcgtcaacaacaaagggaattctatttattcccactgaacaggaagttatagatggaactaataataccaaagcagttacaccagcaacgttggcaacaagattatcttatccaaatgcaactgaaactgtttacggattaacaagatattcaaccaatgatgaagccattgccggagttaataatgaatcttctataactccagctaaatttactgtcgcccttaataatgcgtttgaaacgcgagtttcaactgaatcctcaaatggtgttattaaaatttcatctctaccgcaagcattagctggtgcagatgatactactgcaatgactccattaaaaacacagcagttagctattaaattaattgcgcaaattgctccttctgaaaccacagctaccgaatcggaccaaggtgttgttcaattagcaacagtagcgcaggttcgtcagggaactttaagagaaggctatgcaatttctccttatacgtttatgaattcatcttctactgaagaatataaaggcgtaattaaattaggaacacaatcagaagttaactcgaataatgcttctgttgcggttactggcgcaactcttaatggtcgtggttctacgacgtcaatgagaggcgtagttaaattaactacaaccgccggttcacagagtggaggcgatgcttcatcagccttagcttggaatgctgacgttatccagcaaagaggtggtcaaattatctatggaacactccgcattgaagacacatttacaatagctaatggtggagcaaatattacgggtaccgtcagaatgactggcggttatattcaaggtaaccgcatcgtaacacaaaatgaaattgatagaactattcctgtcggagctattatgatgtgggccgctgatagtcttcctagtgatgcttggcgcttctgccatggtggaactgtttcagcgtcagattgtccattatatgcttctagaattggaacaagatatggcggaaacccatcaaatcctggattgcctgacatgcgtggtctttttgttcgtggttctggtcgtggttctcacttaacaaatccaaatgttaatggtaatgaccaatttggtaaacctagattaggtgtaggttgtaccggtggatatgttggtgaagtacagatacaacagatgtcttatcataaacatgctggtggatttggtgagcatgatgatctgggggcattcggtaatacccgtagatcaaattttgttggtacacgtaaaggacttgactgggataaccgttcatacttcaccaatgacggatatgaaattgacccagaatcacaacgaaattccaaatatacattaaatcgtcctgaattaattggaaatgaaacacgtccatggaacatttctttaaactacataattaaggtaaaagaatgacagatattgtactgaatgacttaccattcgttgacggccctcctgcagagggccagagccgcatttcctggattaaaaacggcgaagaaatattaggagctgacacacagtatggaagtgaaggctcaatgaatagacctacggtttctgtactaagaaatgttgaagttcttgataaaaacattggaatacttaaaacatctttagaaaccgcaaatagtgatattaaaacaattcagggcatcttagatgtatctggtgatattgaagctttggcccaaataggtatcaataaaaaggatatttctgacctcaaaacgctaaccagtgaacacacagaaatattaaatggaactaataatacggttgacagtattcttgccgatattggtccatttaacgccgaggccaactctgtatacagaacgatcagaaatgatttactgtggataaagcgtgaacttggacaatacactggtcaagatattaatggtcttcctgttgtaggaaatcctagtagtggaatgaagcatcgcattattaataatactgatgtcatcacttcgcagggaatacgtttaagcgaattagaaacaaaatttattgaatctgatgtaggttctttgaccattgaagttggtaatcttcgtgaagagcttggaccgaaaccaccatcattttcacagaacgtttatagtcgtttaaatgaaattgacactaaacagacaacagttgagtctgacattagtgctattaagacctcaataggatatccaggaaataattcgattatcacgagtgttaatacaaacactgataatattgcatctattaatttagagctaaatcaaagtggaggtattaaacagcgtttaaccgttattgaaacttccattggttcagatgatattccttcgagtattaaaggtcaaatcaaagataatacaacttcaatcgaatctctaaatggaatcgtcggtgaaaacacttcatctggcttaagagcgaatgtttcatggttaaaccaaattgttggaactgattctagcggtggacaaccttctcctcctgggtctcttttaaaccgagtttctacaattgaaacttctgtttcaggcttgaataacgctgttcaaaacctacaagtagagattggtaataacagcgcaggaattaaagggcaagttgtagcgttaaatactttagtaaatggaactaatccaaacggttcaactgttgaagagcgcggattaaccaattcaataaaagctaacgaaactaacattgcatcagttacacaagaagtgaatacagctaaaggcaatatatcttctttacaaggtgatgttcaagctctccaagaagccggttatattcctgaagctccaagagatgggcaagcttacgttcgtaaagatggcgaatgggtattcctttctacctttttatcaccagcataacatggggccgcaaggccccaaaggattttaaatgtcaggatataatcctcagaatccaaaggaactcaaagatgtcattctaagacgtttaggggctccaattattaatgttgagttaacacccgatcaaatttacgattgtatccagcgtgccctagaattatacggtgaataccattttgatggactcaataaaggttttcatgttttttatgtaggggatgatgaagaaaggtacaagaccggagtcttcgatttaagaggttctaacgtatttgcagtaacccgcattttacgcacaaatattgggtcaataacatctatggatggaaacgctacatatccgtggtttactgactttcttttaggaatggctggtattaatggcggaatgggaacgtcttgtaatagattttatggaccaaatgcctttggagctgatttaggatattttacccagcttaccagttatatgggaatgatgcaagatatgctctctcctattccagacttttggtttaattcagcaaatgaacagctcaaagtcatgggaaacttccaaaaatatgatttaattatcgtagaaagctggactaaatcatacattgatacaaacaaaatggttggaaatacagtaggatatggaacagtcggtccacaagatagctggtcattatctgaacgatataataacccagaccacaatttagtaggtcgtgttgtcggccaagatccgaatgttaaacagggtgcttataataatcgttgggtgaaagactatgcaacagctttagctaaagaattgaacggtcaaattttagcacgccaccaaggtatgatgcttccgggcggtgttacaattgatgggcagcgcttaatagaagaagccagattagaaaaagaagcactgcgcgaagaattatacttacttgatcctccatttggaattttggtaggttaatatggctacttatgataaaaatctttttgctaaattggaaaaccgcacaggttattctcagaccaatgaaactgaaatattaaatccttatgtaaatttcaatcattataaaaacagccaaatattagctgatgtattagtagctgaaagcattcaaatgcgaggtgtagaatgctattatgttccaagagagtatgtttcccctgatttgatattcggcgaagacttaaaaaataaatttactaaagcttggaaatttgctgcatatttaaattcatttgaaggatatgaaggagctaaatcgttctttagtaactttggtatgcaagtacaagacgaagtgactttatctattaacccaaatttatttaagcatcaagttaacggaaaagaacccaaggaaggtgatttgatatattttcctatggataacagcttatttgaaattaactgggttgaaccatatgatccattttatcaattaggccaaaacgctattcgtaaaattacggcaggtaaattcatttattctggagaagaaattaatccagttctacagaaaaatgaaggaattaacattccagaatttagtgaattagaattaaatgctgttcgcaatcttaacggtattcatgacattaatattgatcagtatgctgaagtagatcaaattaattctgaagctaaagaatacgttgaaccttatgttgttgtcaataacagaggcaaatctttcgaatctagcccatttgacaatgatttcatggattaataaatattataaactaattaaagcccggattaggagaagtcatgtttggttatttttataattcgtcttttagacgatatgctaccttgatgggcgatttgttttcaaatatccaaatcaaacgtcagttagaatctggtgataagtttatacgtgttcctattacgtatgcatcaaaggaacactttatgatgaaattgaataaatggacatcaataaattcacaagaagatgtagctaaagttgaaactattctacctcgtataaatttacatttagttgattttagctataatgctccatttaaaacaaacattttaaatcagaatttactgcaaaaaggtgcaacttctgtagtatcgcagtataatccatctcctattaaaatgatttatgaattgagtatctttactcgctatgaagatgatatgtttcaaatagttgaacagattcttccatattttcaacctcattttaatacaactatgtacgagcagtttggaaatgatattccatttaaaagggatattaaaattgtactgatgtctgctgctatagacgaagctatagatggggataatttatctcgtcgtagaattgaatggtcattaacatttgaagtaaatggatggatgtatcctccagtagatgatgcagaaggattaattcgtactacttatacagattttcacgccaatacaagagatttgcctgacggcgaaggtgtttttgaatctgtcgatagcgaagttgttcctcgagatattgacccagaagactgggatggaacagtaaaacaaactttcactagtaatgtaaatagaccaacaccgccagaacctcctggcccaagaacatagaggttattatggaaggtcttgatataaacaaacttttagatatttctgacctccccggaattgacggggaggaaatcaaagtgtatgaacctctgcaattagtagaagttaaaagcaatccacaaaaccgtactccagacttagaagatgattatggagtagttcgtcgaaatatgcattttcagcaacaaatgctaatggacgctgccaagatttttcttgagacagcaaagaatgctgattctcctcgtcacatggaagtatttgcaactcttatggggcaaatgactacgacgaacagagaaatactgaagcttcataaagatatgaaagacattacatctgagcaggttggcaccaaaggcgctgttcctacaggtcaaatgaatattcagaatgcgacagtattcatgggttcaccaacagaattaatggacgaaattggtgatgcttacgaggctcaagaagctcgtgagaaggtgataaatggaacaaccgattaatgtattaaatgatttccatccgttaaatgaagctggaaaaattttaataaaacacccaagcttagcggaaagaaaagatgaagatggaattcattggataaaatctcagtgggatggaaaatggtatcctgaaaaattcagtgattaccttcgtctacacaaaatagtaaaaattccaaacaactctgataagcctgaattatttcaaacttataaagataagaataataaaagatctcggtatatgggtcttcctaacttgaaacgagctaatattaaaacacaatggactcgtgaaatggttgaggaatggaaaaaatgccgagatgatattgtttattttgcagaaacatactgtgccattactcatattgactatggtgtcataaaggttcaattacgtgactatcagcgtgatatgctcaaaataatgtcatctaaacgtatgactgtttgtaatctatcgcgccagctcggtaaaaccaccgtagtagctattttccttgcacactttgtatgttttaacaaagataaagctgtaggtattcttgcacacaaaggctcaatgtctgcggaagttttagaccgtactaagcaagcaattgaactgcttcctgactttttacaaccaggaattgttgaatggaataagggttcaattgaactagataatggttcttcaattggcgcttatgcttcctctcctgacgcagttcgtggtaactcgttcgcaatgatttacattgacgaatgtgcgtttattccaaacttccatgattcctggcttgctattcaaccagtaatttcatctggtcgtcgttcgaaaattattattactacgactcctaatggattaaatcatttttatgatatttggactgctgctgtcgaaggtaaatctggatttgaaccatatactgctatttggaattcagttaaagaacgtctttataacgatgaagatatttttgacgatggatggcaatggagcatacaaaccattaatggttcttcattagctcaattccgtcaagaacatactgcagcgtttgaagggacttctggtacattaatttcaggaatgaaattagctgttatggattttattgaagtaacaccagatgatcatggttttcaccaatttaaaaaacctgaaccagatagaaaatatattgcaactctagattgctcagaaggtcgtgggcaagattaccacgctttgcatattattgatgttactgatgatgtgtgggaacaggttggtgttttgcattcaaacactatttctcatttaattctacctgacatcgttatgcgttatttagtagaatacaatgaatgcccagtttatattgaattaaatagtactggtgtgtcagttgcaaaatcgctttatatggatttagaatacgaaggtgttatctgcgattcatatactgatttaggaatgaaacaaactaaacgcacgaaagcagtaggatgttccacgctaaaagaccttattgaaaaagataagcttattattcatcaccgagcgactattcaagaatttagaacgtttagtgaaaaaggcgtgtcttgggcggctgaagaaggttatcatgacgatttagtaatgtctttagtgatttttggatggttatcaacgcagtcaaaatttattgattatgcggataaagatgacatgcgattagcatctgaagtattttcaaaagagcttcaggatatgagcgacgactacgcgccagttatatttgtggattcggttcattctgctgagtatgttccagtatctcatggtatgtcaatggtataaatatattaaagcatattaaagaggattaaaaatgactttattatctccgggcattgagctcaaagaaactacggttcaaagcaccgtagttaataactctactggtacagcagctttggccggtaaattccagtggggtcctgcttttcagattaaacaggttacaaatgaagtagatttagttaatacttttggtcaaccaaccgctgaaactgctgactattttatgtctgcgatgaatttcttgcagtacggaaatgacttacgagtagttcgtgctgttgatagagataccgctaaaaactcatcgccaattgctggtaatattgattacacaatttctaccccaggtagtaactatgcggttggagataaaatcacggtcaaatatgtttcagatgatattgaaactgaaggtaaaattactgaagtagacgcagatggaaaaattaagaaaattaatattcctactggcaaaaattacgctaaagcgaaagaagtcggtgaatatccaacactaggttctaactggactgcggaaatttcttcatcttcctctggtttagctgcagtaataactcttggaaaaattattactgattctggtattttattagctgaaattgaaaatgctgaagctgctatgacagcggttgactttcaagcaaatcttaaaaaatatggaattccaggagtagtagcgctttatccaggcgaattaggcgataaaattgaaattgaaatcgtatctaaagctgactatgcaaaaggagcttctgcattactcccaatttatccaggtggtggtactcgtgcatctactgctaaagcagtgtttggatatggaccgcaaactgattcacagtacgctattatagttcgtcgtaatgatgctattgttcaaagcgttgttctttcaactaagcgtggtgaaaaagatatttacgatagtaacatctatatcgatgactttttcgcaaaaggtggttcagaatatatttttgcaactgcacaaaactggccagaaggcttctctggaattttaactctgtctggtggattatcatcaaatgctgaagtaacagcaggagatttgatggaagcttgggacttctttgctgaccgtgaatctgttgacgttcaactgtttattgcaggttcttgtgccggtgaatctttagaaacagcatctactgtccaaaaacacgtcgtttcaattggggatgctcgccaagattgcttagtattgtgctctcctccgcgtgaaactgtagttggaattcctgtaacccgtgctgttgataacctagtcaattggagaactgcggcaggttcatacactgataataactttaatatcagttcaacctatgcagcaattgatggtaaccataagtatcagtatgacaaatataatgatgtgaatcgttgggttccattagcagctgatattgctggtttatgcgcaagaactgataacgtatctcagacttggatgtctccagctggttataatcgtggtcagattcttaacgttattaaacttgctattgaaactcgccaggctcagcgcgaccgtttataccaagaagctatcaacccggtaaccggtacaggtggtgatggttacgtattgtatggtgataaaacagctacttctgttccttctccatttgatcgtattaacgttcgtcgtctgtttaatatgttgaaaacgaatatcggacgtagttcaaaatatcgtttgttcgaattaaacaacgcgtttactcgttcatcattccgcacagaaactgcccagtacttacaagggaataaagctctcggtggaatttatgaatatcgtgtagtttgcgatacaacaaataacactccgtcagtaattgatagaaatgagtttgttgcaacattctacatccaaccggctagaagcattaactacattaccttaaacttcgtagcaactgctactggtgcagatttcgatgagttaactggtcttgctggttaatacggtgcattctaaaggcctgtttcggcaggccatataaatacactatatccttaattctttaattctatatgccctaggttaaacatagggatataaatactacagaggctaatatgtttgtagatgatgtaacacgagcgtttgaatctggtgattttgctcgacctaacttattccaagtagaaatttcttatcttggacaaaattttacgttccaatgtaaagctactgctctaccagctggtattgtagaaaaaattccagtcggatttatgaaccgtaaaattaacgtagcaggcgatcgtacattcgatgactggactgttacagtaatgaacgatgaagctcatgatgctcgtcagaagttcgttgattggcaaagcattgctgcggggcaaggaaacgaaattactggtggaaaacctgcagagtataaaaagagcgctatcgttcgtcaatatgctcgtgacgctaaaacagtaacaaaagaaattgaaattaaaggtctgtggcctactaacgtgggtgaacttcaattagattgggattcaaacaatgaaatccaaacttttgaagtaactcttgctctcgattattgggaataaaatgaatggggagaaatccccatcctgcttaaagcagagaagtccattataaatataactataattcccatttggagaatacaatgaaatttaatgtattaagtttgtttgctccatgggctaaaatggacgaacgaaattttaaagaccaagaaaaagaagatcttgtttccattacagccccaaagcttgatgatggagcaagagaatttgaagtaagctcgaatgaagctgcttctccttataatgctgcattccaaacaatttttggttcatatgaaccaggaatgaaaactactcgtgagcttattgatacatatcgtaatctcatgaataactatgaagtagataatgcagtttcagaaatcgtttcagatgctatcgtctatgaagatgatactgaagtcgtagcgttaaatttggataaatctaaatttagcccaaaaattaaaaatatgatgttagatgaatttagtgatgtattaaatcatctatcgtttcaacgaaaaggttctgatcattttagacgttggtatgttgattcaagaattttctttcataaaatcattgatccaaaacgtccaaaagaaggcataaaagaattacgtagattagaccctcgccaagttcagtatgttcgtgaaattataacagaaactgaagctggcacaaaaatagttaaaggttacaaagaatattttatatatgatactgcccatgagtcatatgcatgtgatggtagaatgtatgaagctggcacaaaaataaaaattcctaaagctgccgtcgtttatgcccattctggattagtcgattgttgcggtaaaaatatcatcgggtatttgcatcgtgctgttaaacctgctaaccaattaaaattattagaagatgctgtagtcatttatcgcattactcgtgctcctgaccgtcgtgtttggtatgtagacacaggtaatatgcctgctcgtaaagctgctgagcacatgcaacatgttatgaacacgatgaaaaaccgtgtagtatatgatgcatcaacaggtaaaataaaaaatcaacagcataatatgtctatgaccgaagactattggttgcagcgccgtgatggtaaagctgtgacagaagttgatactcttcctggtgctgataatactggcaatatggaagatattcgttggtttagacaagctctttatatggcattacgtgttcctctttcacgcattccgcaagaccaacaaggcggtgtgatgtttgattctggaactagcattacacgtgatgaattaacgtttgctaaatttattcgtgagttacagcacaagtttgaagaagttttcctagatccgcttaaaacaaatcttttgcttaaaggtataatcacagaagatgagtggaatgatgaaataaataatattaagatagaatttcatcgggatagctactttgctgagctcaaagaagcagaaattttggaacgaagaattaatatgctaaccatggcagaaccatttattggtaaatatatttctcacagaactgctatgaaagacattttgcagatgactgatgaagaaatagaacaagaagccaagcaaattgaagaagagtctaaagaggctcgtttccaagaccccgaccaagaacaagaggatttttaatggaaggtttaattgaagctattaaatcaaacgacctcgtagccgctcgtaaattatttgctgaagccatggctgcaagaacgattgatttaattaaagaagaaaaaatcgctatcgctcgcaatttcttaatcgaaggtgaagaacctgaagacgaggatgaagatgaagatgacgaagatagtgatgataaagacgacaaaaaagacgaagactctgacgaagacgaggatgatgaataatgcttctgatccctgaaactcatgaattagttctcgagaatgtcgaagcacttattcctgaagcacagggtcgctttgacgaattgtcttctgctttaaataaagacgatataaatacaattgtcgagaatatgcttgatgatgaaactgatttagcggttgcattagcttctattaatgaaaatatgccgttaaatgaattcatcgttaaacatgtttctgcccgtggtgaaattactcgcactaaagaccgcaaaacgcgtgaacgaaatgcatttcaaaccactgggctgtctaaagcaaaacgtagacaaattgctcgtaaagctaccaaaacgaagattgccaatccagcaggtcaatctcgtgctcagcgtaagcgtaaaaaagctcttaaacgccgtaaagcattaggattaagctaatgaatgaaccccaattactaattgaaacttggggtcaacctggcgaaattattgatggcgtaccaatgcttgaatctcatgatggaaaagacttaggtttaaaaccgggtttatacatcgaaggaatattcatgcaagcggaagtcgtcaatagaaataaacgtctttatccaaaacgtatattagaaaaagcggtaaaagactatattaatgagcaagttttaactaaacaagctctcggagaattaaatcatcctccacgcgctaatgttgacccgatgcaagccgctatcattatagaagatatgtggtggaaaggaaatgacgtatacggacgagctcgtgttattgaaggtgaccatggtcctggagataaattagcagctaatattcgtgccggatggattccaggagtttcttctcgtggattaggttcattgactgacacaaatgaaggttatcgtatcgtaaacgaaggattcaaattaactgtaggtgttgatgcagtatggggtccaagtgctccagatgcatgggtaactcctaaggaaattaccgaatcacagacggcggaagccgatacaagtgccgatgacgcctatatggctctcgcagaggccatgaaaaaagcgttataaatattattatctaaacaacaggactacaaaatgcttaaagaacaactgattgccgaagcgcagaaaattgatgcttccgttgctcttgatagtattttcgaatcagttaatatttctccggaagcaaaagaaactttcggcactgtattcgaagctaccgtcaagcagcacgccgttaaattagctgaatctcatatcgctaaaattgctgaaaaagcagaagaagaagtagaaaaaaataaagaagaagccgaagaaaaagctgagaagaaaatcgctgagcaagcttctaaattcattgaccatcttgcaaaagaatggctcgctgaaaataaattagcagttgataaaggcatcaaagccgaactgtttgaatccatgcttggtggattaaaagagctctttgttgaacacaacgttgttgttccagaagaatcagttgatgttgtagctgaaatggaagaagagctgcaagaacataaagaagaatcgcctcgtctgttcgaagaactgaatatgcgcgacgcatatatcaattatgtgcagcgtgaagtggcattgagcgaaagtactaaagatctgactgagtctcaaaaagaaaaagtctctgctctggtcgaaggtatggattattcagatgcattctcaagtaaattgagtgcaatcgtagaaatggtgaagaaatctaataaagatgaaagcactattactgagagtataaatactcctgatactgaagcagccggactgaatttcgtcactgaagctgtagaagataaagctgcacagggtgcagaagatattgtaagtgtatatgcgaaagtcgcatctcgtttctaattttaaaggttaacacaaatgactatcaaaactaaagctgaacttttgaacaaatggaagccattactggaaggtgaaggtttaccggaaattgctaatagcaaacaagcgattatcgctaaaatctttgaaaaccaggaaaaagatttccagacagctccggaatataaagacgaaaaaattgctcaggcattcggttctttcttaacagaagctgaaatcggtggtgaccacggttacaatgctaccaacatcgctgcaggtcagacttctggcgcagtaactcagattggcccagctgttatgggtatggtacgtcgtgctattcctaacctgattgctttcgatatttgtggtgttcagccgatgaacagcccgactggccaggtattcgcactgcgcgcagtatatggtaaagacccagtggctgccggtgctaaagaagcattccacccaatgtatggtccagatgcaatgttctctggtcagggtgctgctaagaaattcccagctctggctgctagcacacaaaccacagtaggtgatatctatactcacttcttccaggaaactggtactgtatatctgcaagcttctgttcaagtaacaatcgatgctggtgcgactgatgctgctaaattagatgctgaaattaagaaacaaatggaagctggtgcactggtagaaatcgctgaaggtatggctacttctatcgctgaactccaggaaggtttcaatggttctaccgataacccatggaatgaaatgggcttccgtatcgataagcaagttatcgaagctaaatctcgtcagctgaaagctgcttactctattgaattagcacaagacctccgcgctgttcacggtatggatgctgatgctgaactgtctggtattctggctacagaaattatgctggaaatcaaccgtgaagttgttgattggattaactactcagctcaggttggtaaatctggtatgaccctgactccgggttctaaagctggtgtatttgacttccaggacccaattgatattcgtggtgctcgctgggcgggtgaatcctttaaagctctgttgttccagattgacaaagaagcagttgaaattgctcgtcagaccggtcgtggtgaaggtaacttcattatcgcttcccgtaacgtagttaacgttttggcttcagttgataccggcatttcttatgctgcacagggtctggctaccggctttagcactgatactaccaagtcagtatttgctggtgttctgggtggtaaataccgcgtatatatcgaccagtatgctaaacaggattatttcactgtaggttataaaggtccgaacgaaatggatgctggtatttactatgctccatatgtagctctgactccgctgcgtggttccgatccgaagaacttccaaccggtaatgggattcaaaactcgttacggtatcggtatcaacccatttgcagaatccgctgctcaggctccggcttctcgcatccagagcggtatgccttctattctgaatagccttggtaaaaacgcttactttagacgtgtatatgttaaaggtatctaatctctaacgatagaaacacaattttagggaaccttcgggttcccttttttctattttatacgatagcaatcaggcatatcatccgcatttatccaattgcgaatagttttaggactaactttaaaatgctccgctgcgtaatcaggattatcaaatttaacgccctttatacatattggaataaattttttaataccaccaagtttttcagaaatagctttacgatgtgaaatcgatataggtttgttttttcgtggatgaacatgtgttttataatattcattgcgccctttaactcgcttcgcaatagtttcatcagattgcttaacgcctgtttttgcctttgatatttttcgtttagcttccacagtcattccttcttttgttcgtattgataacatattacgatatgaaggatcttgcaaatgaactataactggatttcctctgccaccaatagcagcattataggtatcagttctcataacgaattcctcattaactagtaaagcttccattttatacatctcctcagatgaggagaaagaataaagaatttcttttttaaagttatgaataccatattttttgatggattttttgatgtttacgccagaacccatataaccatcgttttcgtcaagagtagcatgagctccgatgtaaatttttccattgatgatattagtaatttgatatattaaatatttcattttaaacatcactccgtttgtatatgattataatatcatattactttggtcttgtaaataactttataaatagtattatatttcaacaaggaaaatacaatggctaaaatcaacgaacttctgcgcgaatcaaccacaacgaatagcaactcaatcggtcgcccaaatctcgttgctttgactcgcgctaccactaaattaatatattctgacattgtagcaacgcaaagaactaatcaacctgttgctgctttttatggtatcaaataccttaacccagacaacgaatttacatttaaaactggtgctacttacgctggcgaagctggatatgtagaccgagaacaaatcacagaattaacagaagagtctaaattaactctcaataaaggcgatttattcaaatataataatatcgtttataaagtattagaagatactccatttgctactatcgaagaaagtgatttagaattagctcttcagattgcaatcgttcttttaaaggttcgtctattttctgacgcagcgtcaacaagcaaatttgaaagctctgatagtgaaattgcggatgctagattccagattaataaatggcaaactgcagttaaatctcgtaaacttaaaactggcatcacagttgaattagcgcaagatttagaagcaaatggattcgatgctcctaatttcttggaagatttgcttgcaactgaaatggcagatgaaatcaataaagacattctgcagtctttgattacagtgtcaaaacgctataaagttacaggaattactgatagtggattcatcgatttgagttatgcatctgctcctgaagctggtcgttcattataccgaatggtatgtgaaatggtttcgcatatccaaaaagaatcaacttatacagcaacgttctgtgttgcttcagctcgtgccgctgcgattcttgctgcatcaggctggttaaaacataaaccagaagatgacaaatatctttcacaaaatgcctacgggttcttagctaatggtttaccgctttattgcgatactaacagcccattagattatgtaatcgttggcgtagtagaaaatatcggtgaaaaagaaattgttggatcaattttctatgctccgtatacagaaggtctcgacttagatgaccctgaacatgtaggtgcatttaaagttgttgttgatccagaaagcttacaaccatctatcggtttattagttagatatgctttatcagcaaatccttatactgtagcaaaagatgaaaaagaagcaagaataattgacggtggagacatggataaaatggcaggtcgttcagatttgtctgttttattaggtgttaagctaccaaaaattatcattgatgaataaaacaaagggacctttcggtccctttttatttaacttaccaactcaatccaagctggacgaagtacatcttgtaccattttaactaattcctttttaatcaaagaaggattatctgcttgagttagagtaataccttcacgagaagtttcttccaaaatatcttgaacagttagccccatcacctttccaaaatcctttggaccaatttcgccaattttagaaataacgttatttacgcggttcagtgtaacgtaacaagctaaaattcccaccaatttgttatcagcttctgatagctcaactttagctttaataggcttatcagactttttcttttcactaaatttagagttcttgcatttaatcgctacacgatttccattacgaagccaagaaggataacaaggtttcaatacatatccttcagcagtaaatacttcgccttttgcttcggcattccaaacgcatttatttgcatcaactaatccagcatggtctactgtaaaattataatcttggacgacagaatctaaatcatttggcaatttaataagctcttcaaatttaccgcgacctaaaagtggagccattttaaatttaaatgtattacagaatgattccatcatataatcatctacataagtcacatcaccgctttctgtagtaacaataatgtcaaatacataaaaatctttatcacaataatcaacattcttctgaatgccaggtccagcgaattcgccaaagacttgataagatacaaccgctgaggtttccataatatcttgtacagctttaatggaatcagcataattcttcaaaataatttcatacccaaagaaatcttcagcaggaagaatcggtccagtgcgtttagcgcaagtcactttatcacgctcaataatcaatgagaaatttgtgccgtgaatcttttcacgagctacccactccccaccagtcaatcccaagctataaagtttttcaataaatttagagttgtaatgattttcaagactgctatactttttaaacataattaatcctcaaaatgtaatttctaaccaatcaccatcacgctgatcactattgactttaaagctgaatccttcttttctcagccaatcaccaatttcttctgtaatcaatttatcacgagcaatacaataataattaaaatgtgttttaccttgttcagctgctttattagcaagttctgaaaaatctttaataaaacactctagcttaaactgtttactttttaatgctttttcgcgtaattgattagcaaaagattcattttcataaagatcatactgttccatttttcacctttttattgatatgtctttttctatagacaactttttctcgagcccataatacagccacttcttttgcctgtaagtttaattcacgagcaatttcaatgaatgactttccagactcatgaagagtaaacaccacaacctcagttctcataatcaatctcatgttatcgagttggtgccattatatacatcattttctgattgtgttttgtgtgctttcaaaatgaagaaaggggccgaagccccttatgattatggataggtatagatgataccagtttctaaagcagttttatgaatgatgtatccattacgcgattcttggacatcaacttctggatagtctttcatcatcttctggagagtgtaacgatgcaggtaatatttactatctgggtcgtcagttttccaatctttaccttcttcggtcattttttggatttcatccataacccaccaaccgcaccagatgtaagctgagctacggtgtggaagaggatgaacataaggtaattcaccttctggttctggagcaactttagccgtgactgttacattaccagttttggtaacggttacagggttataatctgcagcagtaacagttgcagtaacttcaatagtttgacttccaacagatgaggtatcgacagtatatacgttagttgacccttctacaggagaagaatctttcttccatgagtaagtaatttgtgcttcttctggagcacccgtaacattagccgtaaatgtagccgaagcatcttgctgaacattaatagaaggaggagtcaatgtaacctgtggattcattgtcttcttattaaccgttaatgatacttcattagaagtaacgcttagtgcatcataatctgtcgcggttacttgggctacgcatttaattctttttactccacttgtagttggagtatagctaaatgtagagttagtttctccaccaacttgtgaatcatctacataccactgatacgtagcagatgctccatcaggttgagaagctaaggcagcagtaaattgaactggggttccaatcactccagccgcaggactagcaggagttacggctaaggtagtcgtctgtgtcttatttttaactgtgatagttgttgtcgcttcagccgtttccgggcctccttcagaaagtgtatttgttgcaactactttaatagtcttttgaccggcaggtccttttagtacataactaaaagttgcttcagctccatcttgtggaacattatctacgctccaagcatatgtaatagttccgcctccagtttgaccactgggtgtagcagtaaactgcttagtttcatcaataacccctgtaggtgttttaggagttatatcaactgtaaaagtcataagttatccttattttaatgttacgaaagaagagttgcgtgtttcacgaattaaaactgatccatcgcgattaatgtaataaattaagctaaataaagtttggtgtgctgacgcatgttcaaaactagttgggtgagatttccaatcaggagtttcagcaatccattgataaatccaccaaggaacagtacagaatcctagattttttccaatcagttgaagattcggactaaagttttccggaagagtaaatacagacggcttttcagattcaataatctcagctacagcctgttcgaatttttcttcaacaaaaggagtatcttcaatcaaaacatcggtattttcaggaattttatccgtttctactacttcaattttaatgtcagatttaatcggagaatcaatcagaagtgctgcttctggattgacttcttcatcgtcatattttaatccctctgcggcatcagcagcatcaattaagtctttaatagataacccatcagtctctggcataggttcactagcgagcttctggagggcttcttcaatatcaacaacgatattatcaaaagatttattctttttgacctttataccaaactgttcagcatattcagctaatttagctttagcttctttgttatcatcaagagccttcagctctgcaatataatctttatctatcataatatttcctcagtataaatatagatatatttattactcggaaaatagtatgtaccactttgtatatgaaacaacaaatctaataaatggtaaaaagtatataggaaagcactctactgatgacttgaatgatggttaccttggttccggtaaggcaattcagcaggctataaagaaatatggtgaaaacaatttctctagaacaatactaaaagagtttaaaacttccgaagaagcgtacatgtatgaagaagaaattataactcctgaactaataaaaagcaaaaattattataatatgaaacctggtggaattggtggaattgttatgactacagatgttatagcaaagatgaaagaatcttccgctaaaagatttgaaaactcaccgggcacggtattaggtaaaacttgttatactaatggaactaaaaatatttttattaaacctggagaacttgttccagaaggatttgtaaaagggatggttcatcctaatagaaagtccagaaaaggatgtaaagtcaaaccgactaccacaggaactttttgggtcaataatggcgcaataaataaattaatacaaccagacggtattattcccgacggatttattaaaggtcgtctcatgaaaagagattctaaaggcaaatttagtaaggcataattatggatattaaagtacattttcacgacttcagtcatgtacgcatcgattgtgaagagagcacgttccacgaattaagagatttcttttcgtttgaggccgatggatatagatttaatcctcgcttcagatatggcaactgggatggacgaatccgtcttttagattataatcgtcttcttccattcggcttagtcgggcaaattaaaaaattctgtgataattttggctataaagcctggattgacccacaaattaacgaaaaagaagaattatcaagaaaagattttgatgaatggctttctaaattagaaatctattcaggaaataaaagaattgaaccgcactggtatcaaaaagatgcagtgttcgaaggattagttaatcgtcgtagaattcttaatcttccaacatctgcaggtaaatctttaattcaagctttgcttgcgcgatattatttggaaaattatgaaggtaaaattcttatcattgttccaacaactgctctgacaactcagatggctgatgacttcgtcgactatcgtttattcagccatgcaatgataaagaaaattggtggcggagcatcaaaagatgataaatataaaaatgatgcaccagtcgttgttggtacatggcaaactgtagtaaaacaaccgaaagaatggttctcacagtttggaatgatgatgaatgatgaatgccatcttgctacaggaaaaagtatttcatctatcatatcaggtttaaataactgcatgttcaaattcggtttgtctggttcattacgtgatggcaaagccaatatcatgcagtatgttggaatgtttggtgaaatatttaaaccagtaacgacttctaaattaatggaagatggacaagtaactgagctaaaaattaatagtatttttcttcgctatcccgatgagttcactactaaattaaagggaaaaacttaccaagaagaaataaaaattattactgggcttagtaaaagaaataaatggatcgctaaattagctattaagcttgcgcaaaaagatgaaaacgcttttgtcatgtttaaacatgtatcgcatggtaaagctattttcgatttaattaaaaatgaatacgataaagtttattacgtatcaggggaagttgataccgaaacccgcaatataatgaaaaccttagctgaaaatggtaaaggaataattatagtagctagttatggtgtattttctactggtatttcagttaaaaatctgcatcacgttgttttagcgcacggtgttaaatcaaaaatcattgtattacaaacaatcggtcgtgtattacgtaagcatggttctaagacaatagcaacagtctgggacctcatagatagcgcaggcgtcaagccaaaatctgctaatacgaaaaagaaatatgttcatttgaactatcttttaaaacacggcattgatcgtattcagcgctacgcagatgaaaaatttaattacgtaatgaaaacagttaatttaataagcttcggccctttggagaaaaagatgttactagaatttaaacaatttctttatgaagcttctattgatgaatttatgggtaaaattgcctcttgtcaaacattagaaggtttagaagaacttgaagcttattataagaaaagagtcaaagaaactgaattaaaagatactgatgacatctctgtgagagatgctttggcaggaaaaagagctgaattagaagattcagacgatgaagtagaagaaagcttttaaattaaaaaaggcccaaccaaaaaggaagggccaaaactatagactaaaggtcacactatagcaaaagttgtgtttcatttaattgttcttccgaactttctgaaactggtagttctttaatgtaattatagcaaggcccaggatgtacaggacctttgtctgtttcaacaaccaatgcagaatcgattggagttttacagacaacacaaatcttatctgacatgattgtctcctctgaattatatctatttatacaactctcatatgcatatcaatgcccatatctttagaataaaaatattcatcaagatatccggcaaattttcctttaatataaaggacatcttcaccacacgggtggtcggccaggatacgaatatcctgacgcttaagattatgctttttcattaagaattgaatttccgtttcaaattcttcttcataattaaaagcatcatcaatgctatatctcattattttccagcctcaaatgctcgcatgtcttgaatatgcttaatagcaaatccacgtgatttaatagcatcaagagctccgctacagaaatctaataaaatcccccaatactgcaacgaggtatcaacctttaaaacatccttatccgctgatagaactgtcttcatttctgatttctcgtaacgatccatactaaattcatcaccatctcctcgtcccgagtagtagtctaatctagctttaagagcaacttttttctgtgcttcaattctaagcatttcctttttaatacttgaatgcttattaagccatttactatataacatcacattattagctgcttcatactgtaatttagtcgaatctataaacacatctttcttcaattcttcttgaagatcttctaatctcatattgttctctattcaattgttattggttgttattggatggacttagattcattataccacgttttaacgtgaagcattatactctattactggaagccagctgcagttttatctgctcaatatcatcaggattatcgatgaccgaaaagcgtatttctactatcagagtataatcgtcataaacgggtatcacattaactgctaatttatcaatacgtggctcatagtttcttactgcgctttcgatattgcgttcaaccgtgtcagcagtaagaggagtcatattttcaaaaagctggtctgataaatcacatccaaattcagggtcaaacggtcttgaaccttttcttgttgtaataattcccaaaagactgtttttaattgaccttaatccaagcgatctggaaacgtctttgttccaatccattttcatttccgggtcaatatcagaataaagcttattaatatttgccattatagtaactcaaagaactctttgaggcctcttattacgtgagcatgggtttttccacactctggacacttaattggaacagccaaataaacggtaggctttaaaagcatatcttttatagctacaatatctgactctgtgatgatagaatataaatcttctagttccttttcatttaagtcttcaactggaatgctttccccgttagcatgaatcgtttctatacatgatactatcatgtgggctatatttttatcatcaaaaattttagggtatcggaatttaattttaatgtcacctagtgtataccagaggtcttctggtgcatctatttgtgtatgtaatagatttatatgggttggtatttcagttccacaggtgcacttccaggagttttcgtgattaacttcaccgagagaatgtgcccataaatgaatcaacaatagttctgattcttggcggtttaaatcttttgcatttgtgcagtctttgattagctttttaacaattacttctacggaaccattatttttggcagtaataagttctagatattctttaagcgtgaatgcgcgacaattgattattttagaaccaactctcacatcaaatttgtattcatacatatttagctcctttatttatcatatttataaatagaataaaaggagcatctatggcaaacattattcgttgtaaattaccagatggtgttcatcgttttaaaccatttacggtagaagattatcgagattttttgttagttcgaaacgatatagaacatcggtcaccacaagaacaaaagcaaataattactgatttaattgatgattattttggagactatccgaagacttggcaaccatttatatttttgcaggtatttgtagggtcaataggtaaaactaaaagtacggtcacatttatatgtccaaaatgtaaaaaagaaaagacagttccatttgaaatatatcaaaaagaattaaaggaccttgtttttgatgtagctaatgttaaaattaaattaaagtttccttctgagttttatgaaaataaagcaaagatgattactgaaaatattcattctgttcaagtagatgaaatatggtatgattggaaggaaattagcgagtccagtcaaatagaactagttgacgccatcgagatagaaacattagaaaaaattctcgatgcaatgaatcctattaatttaactctacacatgtcatgctgtaataagtacattaaaaaatacactgatatagtagacgtgtttaagctattagttaacccagatgagatatttactttttatcaaattaatcacacactcgtaaaaagtaattatagcttaaattcaataagtaaaatgattcctgccgagcgcggattcgtattaaaactgattgagaaggataaacaataatgagtatgttgcaacgccccggatatccaaatctcagcgttaaattatttgatagctacgacgcttggagtaataatagatttgttgaattagctgctactattaccacattaactatgcgggattctctttatggccgaaatgaaggaatgctgcagttttatgattctaaaaacatccatacaaaaatggatggaaatgaaataattcagatttctgtagctaatgcaaatgatattaataatgttaaaacacgaatttatggatgtaagcatttttccgtgtcagtagattcaaaaggtgataacatcattgctattgaattgggaactattcattctatagaaaatcttaaatttggtagaccatttttccctgatgcaggtgaatctataaaagaaatgcttggtgtcatttatcaggatcgcacattattaactccagcaataaatgctataaatgcttatgttcctgatattccatggactagcacatttgaaaactatttgtcatatgtaagagaagttgctctagctgtaggaagcgacaaatttgtatttgtatggcaagacatcatgggcgttaacatgatggactatgatatgatgataaatcaagaaccatatccaatgattgtcggtgagccatctttaataggtcaattcatccaagaattaaaatatccattagcatatgatttcgtttggttgactaaatcgaatcctcacaaacgtgacccaatgaaaaacgctactatctatgcgcattcatttttagattcttcaataccaatgattactacaggaaagggtgaaaactctattgtggtgtcaaggtcaggtgcttattctgaaatgacttataggaatggatatgaagaagctattcgtcttcaaactatggcacaatatgacggctatgctaaatgttctactatcggtaattttaacttgactcctggtgttaaaattatttttaatgatagtaaaaaccaatttaaaacagaattttacgttgatgaagttatccatgaattatccaataataattcagtaactcatctatatatgttcactaatgcaacgaaactggaaacaatagacccagttaaggttaaaaatgaatttaaatctgatactaccactgaagaaagtagttcttccaataagcaataaagaagtttctattcctaaaatgggtcttaaacattataacattttaaaagatgttaaaggtcctgatgaaaatttaaaacttctcattgattctatttgtccgaatttatcaccggcagaagttgatttcgtttctattcatttattggaatttaatggaaagattaaatctcgtaaagaaatagatggttatacttatgacattaatgatgtttatgtatgccaaagattggaatttcaataccaaggaaatacattttattttagacctcctggaaaatttgaacaatttttaacggtgagcgatatgttatctaaatgcttacttagggtcaacgatgaagttaaagaaattaattttcttgagatgccagcattcgttttaaaatgggcaaatgatatttttacaactttagcaattcctggccctaatggtccaataactggaattggcaatattattggattatttgaatgaaaaagccacaagaaatgcaaacgatgcgtagaaaagttatttcagataataaaccaacacaggaagcggctaaatccgcttctaatactttatctgggcttaatgacatatctacgaaattggatgatgctcaagctgcttctgaattaatagctcaaactgtcgaagaaaaatcgaatgaaataattggagcaattgacaatgtagaaagcgcagtgagtgatacatctgccggttctgagttaattgctgaaactgtcgaaattggcaacaatattaataaagaaatcggtgaatcgctcggaagcaaattagataaattaacaagtttactagagcaaaaaatccagacagctggaattcaacagactggaactagtttagctacggttgaaagcgctattcctgttaaagtcgttgaggatgatactgctgaatctgtgggtcctttattaccagctcctgaagcagttaataatgatcctgacgctgattttttccctacccctcagccagttgagccaaagcaagaatcaccagaagaaaaacagaaaaaagaagcatttaacttaaaattatctcaagctttagataaattaacgaagactgttgattttggatttaaaaaatccatttcaattactgataaaatatcaagcatgctatttaagtacaccgtcagtgctgctattgaagctgctaaaatgactgcaatgatattggctgttgttgttggaatagaccttttgatgattcactttaaatactggtcagataaattttcaaaagcctgggatttgtttagtacagactttaccaaattctctagcgaaaccggaacttggggtcctttattacagagcatctttgattctattgataaaattaaacaactttgggaagcgggagattggggtggattgacagtagctattgttgaagggcttggaaaggttctttttaatttaggtgaacttattcaattaggtatggctaaattatctgcagcaattcttcgagtcattcctggtatgaaggatactgctgatgaagtagaaggaagagcattagaaaatttccaaaattctactggagcatctctcaataaagaagaccaagaaaaagtagcaaattatcaagataaacgaatgaatggagaccttggcccaatagcagaaggactagacaaaatctctaactggaaaactcgtgcatctaactggattcgtggtgtagataataaagaagcgctgactaccgacgaagagcgtgcggcagaagaagaaaaattaaagcaactttcaccggaagaaagaaaaaatgctttaatgaaggctaatgaagctcgtgctgcgatgattcgttttgaaaaatatgccgattcagctgatatgagtaaagactcaacggttaaatcagttgaagctgcctatgaagaccttaaaaaacggatggatgacccggatttaaataattcaccggcagttaaaaaagaacttgctgctagattttctaaaattgatgctacttatcaagagctcaagaaaaatcagcctaatgccaaacctgaaacttctgctaaatcaccagaagcgaaacaagtccaggtgattgaaaagaacaaagcacagcaagctcctgttcaacaagcatctccttcgatcaataatactaataatgttattaagaaaaatactgtcgttcataatatgacacctgtaacgagcacgactgctcctggtgtatttgatgcgactggagttaattaaggaataatatggcaattgttaaagaaataactgctgatttaattaaaaagtccggtgagaaaatttcagccggacagagtactaaatcagaagtaggaactaaaacatacacagcccagtttccaactgggcgtgctagtggtaatgacactacagaggacttccaggtaacagatctatataagaatggattattatttactgcatacaatatgtcatctagggattctggaagtcttagatcgatgagatctaactactcttcttcatcttcgagtattttacgtacagctagaaacactattagtagtacagtatcaaaactatcaaatggattaatatcaaataataattcaggaacaataagtaaatctcctatcgcaaacattcttttaccgagatctaaatctgatgttgatacatcatcacatagatttaatgatgttcaagaaagccttatcagtagaggcggaggtactgctactggtgtgctaagtaatattgcttcaaccgcagtatttggggcactggaaagtataacacaaggtataatggctgataataatgaacagatttatacgacagccagaagtatgtatggtggtgctgaaaatagaactaaagtgtttacatgggatttgactccacgttcaacagaagatttaatggctattattaatatctatcaatattttaactatttttcttatggtgaaacgggtaaatctcaatatgctgctgaaataaaggggtatttagatgattggtatcgttctacgttaattgaacctttatctccggaagacgcagctaaaaataaaacactatttgagaaaatgacatcgagtttaactaacgttctagtagtttcaaacccgacagtttggatggtgaaaaactttggcgcaacatctaagtttgatggaaaaacggaaatatttggtccatgtcaaatacagagcattagatttgataaaacacctaatggtaactttaacggattagctattgctccaaacctccctagtacatttactctcgagattactatgagagaaattatcacgttaaaccgtgcttctttatatgcggggactttttaatgtattctttagaggaatttaataatcaagcaataaacgcagatttccaacgtaataatatgtttagctgcgtttttgcgacaactccatcaactaaaagctcttcgttgataagttcaattagcaacttttcttataataacttgggcctaaattcagattggttaggattaactcaaggtgatattaatcagggaattaccacgctaattacagctggcacacaaaaactgataagaaaatcaggagtcagtaaatatcttattggtgccatgagtcaacgtacagttcaaagtttattaggctcatttacagttggtacatatttaattgacttctttaacatggcatataactcatctggattgatgatatactctgtaaaaatgccagagaatagattatcctatgaaactgactggaactataattctcctaatattcgtataaccggaagagaattagaccctttggttatttcatttagaatggattcagaagcttgtaactatcgtgcaatgcaagactgggttaactccgttcaagacccagtaactggactgcgtgctttgccacaagatgtcgaggcagatattcaggttaatcttcattctcgcaatggattacctcatactgcggtgatgttcacgatgcattcaatatcagtgagcgctcctgagttatcatatgatggagataaccaaataactacatttgatgttacttttgcgtacagagtgatgcaggctggagcagttgataggcaacgtgcgcttgaatggcttgaatctgctgctataaatggtattcaaagcgttctcggaaatagtggaggtgttactggactatctaattcgctttcacgacttagtagattagggggaactgcaggaagcatttcaaacattaatactatgacaggaattgtcaattcgcagagtaaaatattaggagcaatataacaatggggaccgaaaggtccatatttttatttacggaatgaaatgaaagcagcaactgaagcaactaaactgtcttcaatataaacttcaatttttacaggagcttctgactcaaatttacctgttactacaccctgaaaaatactttcagtctgttctggctttgaaaaattttcagaaggaaaaattccgaactttttatctgttccaaaaattttgataaattcatcgtaaaccgcttcgttaaaagcattatcagcaggaataacgccttcaactacaagttcttgacctaaaaagcgtaaagaagatttcattttgtgttcctcatgttatgttagtaagactactataacacaacacgagggacttgtaaactacattttgaactttttagtacgcgtaataggcatgcgtcgattttatactgtttcattgtttgaagagcagtatcaaaaacagcattaatgacaccaattggatttccacccaagtttttaagcttaactaatgatagtttctcattaacacctatcattacgatatgcatcattttatcgcctggtttaacgttttatttgtatcaccgccagaggtgtatgtacaaaatctaaatccaggaagtacactttcttcaccggcttgaatagcaaaaatttgtggtattttatgcttaggatttaatgtaacaaccggcgccgaagcgccgtcaaataattctgtaataagttccatgatttatccttgaacgaacttgtaaggcatgtttgcaatatctatgcaagacgcaataattccaagagatgattctactttctgtttaacatttgatgaaacaaatgatgcaaagtcaactctatcttctatatcacctgtcattgtaaccaattcaccagtttccattaaatggtctccgtcatagattaccgattcggtgagttcttctgtggtcataacttcagcttgaataagcttgttgttagttttaactgttccatcattgtaagatgcatcggttattttattaactttgaccattaatccacgtggcaaaatgacttccatttcatttgaaggcgctaaacttccaccgggtaaaacaacattgaccttatcagccccagtaataacccatccaattccaactaaattatcgctagaatttacaagtccttcatcagttttatcaatagaaacgcttaaacgcttttcgtctggtaaaacacctatagatgaatcagtcatccaagtaccaaaaatatttggatataatgatgttgacacaaagtttctaaaataaaaaactcgattttttaccattgcttcgtatattgaaggtaacattcgttgtgaacgatacaaagtaataccttttggtaatcgttcaccatttttaaaggctgaatctaaattatcaatagctttttctatgtcagatgctgtcaaaatacttgtacgctcatctggattatataatcccaaaagagcattatttatgtctacatatcctgaacctacgtattcacgaattccgcgtttttgcgctggtgtgtatttagatgaatctttattttcgactataggatgtaatgaccatccagcggtaagagcatatccacgtaattctcttcgaataatctttgtttttattgcattccaagaattttgtactaactcatttgcagcatcttggtttaaatgcgaatgtttgttaatatttctttcaaaccaagcacccttatatttttctaaagtatcatcgacgatagaagcaatagtttctaaggtttttattgaagtaatagattcttttcgtaatctttctaaagctttattttttatttctgcgttaataatggattctttatcttcaggaattatagaagcttctctgaatgccatcccagctgtaacactgctaagagcattctctagtgcaaatcctgaagtagaaattaccgttaattcattagaatcagaaattaaaggcgcgtccgctggtttatttaattcggccgctgaagcctcaaatttttgaaacattggtgtttcaaatctagaagattccaatgactgactttgcgcaattgctctacgggaaattttaactttaacgattacagcttggtcagaacgtttatcattttcttgcgcaatagatgctgcaattgcctcatttttagttacttgagccccagtatctttattgatataaacatcaccgaccttcgattcaactttagtaaagagctcggtactaatttccggaactcctggaatgtcttctagtgatacatttttgcgatgtataagaatatatgcatactttttatcgtaatcccagagttccttaagaaggacgtatctaccacctgaacgactacggataagtctatcagcaataacttgaatttgtcgagcttggccagcagttttagacttaagaatacggagcatacaggcatcaattttatactggcgcattgtttgcattgcaacagtaaaaactgaattgatataattaattgggcttggaccaagaccttttaatttagcaattgaacctttagcagttaatgtaaaaggaacaatatgcatcattttatcgcccatctttaaatcacgattagtatcacctccagatgtataggtacataaacgaaagcctggttgttcaattgcatcatcaacatgaactgaaaaaatttgcggtattttcttctttggatataagtttgtaattggaagagtagtatcttcgtcaaataattctgtaataagttccatcatatcctctctagtgtttattctattctatttataaaattaaaggcccgaaggcctttaataatctattggtaagagagtacgatatatttcaaactttggacctttttcataagcatcaaatgtttctgtgaatttattataagcatatgcatctataaattcaatcatgatttgtgatacagaagtagaaacatctccaccttctttttgagccacgacaattgtttctaagtaagctttcatagaccagttacctcatgaaaatcaccaaatacatcttcgaatgtattagctttagttttatcttcacgtaaacgaatcgcaatcggaagaaataatttaacgtaatcagtgcggccatcagattttaaccaaccgttgcattcgcactctagaatttttccaatataataattttggttttccataatgcgagtacggtcaagttcatgcgattttacaccggctttatcttttaagcctgaaccagcatttaccttaatttttccacactctgactcaagaataaatccacccgctttagtagggtctttacggtgaggataaattcctacaatttttaaatcaacatcaattacttctttaaatttataaagattttttgaacgagcattttcccataatccatcgatatttttgagaataataccttcaagaccttggtcaatatactttttataaattaccttagcttcatctaggttatttactacctggttttcaattaaaattactttatcatatccagatgtcatttgttctagtttagaaaaacgtacatcatatttcaaacgaaatgcaggaagactgtatatttctaccaacgggacataatcccagacctgaaacttcatgcattgtgcttctttttcagaaatggttccctttaaagatttattggcgattccattagaagcagtacgtgattcagctacttcggcgaattctttagctttactgttttcaggataagcatcaaaaagaaaatctaggccttctggctcctttttaacttgctcatggtataccaattcgccatcaatcaacacaccttctggatgaatctggcgggcttcagcggtcattttaattaactcttccttaagaagatctaatcctagatattcattaccagctcgtgataaaagacgaacatcatctaattcatcacctctaacttcagcaaaacaccgagctccatcagcttttaactgagcaaaggctggaaatttgatattcttattaatgcctttttcatcataagaacttgcgagcatttgaggttgttcaggaattaaacctggccaaactttgtttgcaatagatactgaagcaccacattcaaggtctcgcatcatcactcgacgcaaaacttcaacatcatcttttttaccatcggtgatatatccagttaattcctcaattgctgcatttccagtcaatttccgagtagctaatgtgaattcaatgaagtcaagcatatcggtaagagtcaacattccaaaactctgggtagcaataccaggtttaggccatttcttgatataatactgtaacccacgagaataagtcagacgatatactcgtttaagcaattcattatctttattcttttcaagaattgcttgcttctgtttagttgaaccaatagatgctatttcgttcagaattttaagaatcattgttcatcctttagagtttggtttacagctctattataaatcaattcatcattaagctcagtcaaagacctgtggtacgtggttctaactttatttccttgcatccagtgcttgatataaatgaaaccttgctctacacattttttaaaaattcgttcgtctttttgagctcggaattctggattgcatctaaaaaattgatttacgtgaccgtaatcacgtgtagtattaccttcattttcataaatagtgtgaacaacaaacattagaatgctccttggaaaatattatcaccacaagtaggtctattatacaaatactctataccgccgggcttaatatagttccatgtctgaaacggatgcgtctgatatggatgatatggattataaggattaaatccaggagttctccaggtagtgttccaaggaaaagaatcgtttttaatcatcttttcaattacatcttttatagctttttcactatcaaaactttctttattttcctttggtgaaaaaagcttattctctacatcgttccatgtataaactcgctgagcagtttttggaatattgtcacgctctcctcgagccatccaatacacaggaacacgtaatatttcactcgcgtgatcgcagtggtgagcgagatcgtcaatataacaaattacgttatatttctcttttgcttttttgaacaactcttcttttgaagaatcatgactacacatcagtacttctgagaaagcaccaggaaaaagagcattcaaattaaattgacgatttaatagagcgtcaatagaatcacccaatgctgtaacagcaacaaaattataatcttcttttaatttgttaattacacacagagcatctttatatggagacaagtaacgaataaaatccgaacgattgtatttttcaattaacttgacgccaagttcttcatcacagttaaagagtttaccaggagaaataaatttctcatcttggatcatttttaaaatatgttctaacggaagattatatttctgagcaaaataaggaaggcctgattgccagcttaaacatactccatcaatatcagttaaaatagtaggcttcatagagagtctcttaataggtttaacacatcaataaattcagcttcggttagtattgtatcatcttttgttagaccactagcaatgctgtgcttcaaaacttttcctttcgaggcttgtaatgcatcacgaaagcccttgttttgaatcgctgcttcaaaatatgcatttgtgtataattctttccacgccggggagtatcttgaaaacggaactccaagccaaaagagggtcccacggtcctgagctctagcataagaccttccagcttgttgggcggcaagcccggataacccaaatatacgacgttgttgttcaacatttttcaccttacacccttggaggaatccttcaagacctccaaattgaataccatccataacgaaaggccattgggcgaaattacttaatgcacatgatggccacctaaaattgcttctaatctctaactcagacattttcaatgcttataatttcaacatcagcccaatgaccatagcaaggaagacgaaattcaactggccagttagggtccctttctaatataatagactcaacttgtggttcttcgtgttcatcagtatacggatttttatctgttactttatatgtgaccttaatgtactgaattccaaaaatcttattaattatattcatactaattcctttaatccgtagataggagataattcatcacccatacgaaggtcttcatttccatctacccaggaaacaatataagcctcttttatttgaataccactccatttaaatggaggtagaaccttagaaattaatccaggtataccaactccctttaattcaacagtttgacctaaaaagaatttcattagaacctcatctgaaaaccgtgcgatttaacattaccgccgccaatatcagaaatgttaatttcacgcgcaattgaagggtcaatgtcaatttcacgattgagtttagtgatagctaaagtatcacgcccatttacagtacggaactctaaaggacaaaccacatcaacgtagtttttaatcgattcgctagtaagttgcaaatgtgcaggaaggtctttaggtgcctttgagaaaaccacttcacagaaatttttgcgggtatcaaaataagtagtcataaacataatattttcctcagtaaggggctgaagcccctcattttattttaaatatcaaattcattaagaactacatcaaagattgcttcaagatgctcaggtttagctctgttactcagaatatgacgaatccaagttttaactaagagtttacgattagcaccattccagcaaggatgagtccctaaatcgcgttggcggaaatcatcatccagagcgattttgaagttggaacctttcatcgtgattgaaaccgtgataccgttttcaaatcgcatataaacgtagttaggagtcatatactgttcaatttcgcatactgatccattttgatgtttccagaggcaaatagtatcaatagaacctgcaataccattagaaacatatttacgttcaaagttaatgtagttcatttttattctccgagatgtttaattgcggtacaggtatataatatcatatcctgtaccaaagtaaacaattattttactactttccaatgctgcatgtcaagtttaccaacttttttcatcttctcaattaagcgttctgcacgttggcgagctgtaacataatgccattcgcctaattcattttgttcaatttttccaacgattactgtattcaattcataaatccaaccagtaaagaaattatgaacttgaattgtaaaggtgaaatctgttcccataccttctgttgtttctacttcaataatatcaccttcaactgccattaagaaccacatagtttcatcatatttaccattgaagcatttagttttaactgcagcgttcagattaatcgttttcattttattctcctttgtttgtgtaagataatactatcacaaaggaactatactgtaaacaactttgtgcaatctttggaaaataaaaaaggactcccgaaggagtcctcaacttatgctttctgcttaccaaaacgagaagcatcatctcgaagaaccgcacgtgctcggcgcatgatcttctcaacagtttgattgatacgagagttcgacccacgcttgtagccagcgcgtttagaatcaccaactttcttttcaactgctttctttgctttagcttgttttgccattataaattctcttttaaatgaaaatgcaggacttattggcattgcctgcgcaagccctcaaggggaacataggtttttggatatttaacgaccaggataaccataaacccgtcatcattcacattcaagaggtacaccgtaaaactgtcggggtcttaaaactataatgattcgcaaatcattaatcagacagttcgacggctcctcgatttagctcacactaaggcagtgaatctccaataaattacttcagtgttaccacaaagtgacgaactgcttttcgtgcagcagaagccagaggcttagcatatttaagttcatctttttcctgaagctcagcagctaatgcagtttgagcaggattcagatgtttgaaataacgcaggatttcaagagcttcggcttcaacatcaatagatgcgccatagttttcgtgaccattattccatgcgtttcgttgcagttcaagagcgtgttgtaattgtttaatcatttaaaaattctcgttagagattaaaactcggtagtcacgttcttctgaatttcatcttctttcgacagatctctcagttgtagactaccacatagaattgttcggttaacttattattccgacacccaattcatattattatttatatcacttataaagacacggaatagctttatagtgacaggtaacgaatttttgtttaatttcttttggctgcttaagacccagagctacaaaaggatgcggaacatttcgaatttgaccaactggaagagaagtcaaatcaccaacttcgcagaaaccttcaggaacatcaggaccgacagagtgaactacacacagttcaggaacttcaccttgaacacgtttaccgataataagtcctgattctgtaacttcttcatcaccggcttgtgcaggttcagaaactaaaataacatattcaccgacagcacgaattggtagctgttgtacttcagacatcgtttttcctttttgttaacagatgaattaataataacaaatagttcttaaagcatttatttaccaataaattgaagcaaatgctcaactttcataccattaacggaaatcaatttgtcaatagaaaaacctcgccacgcaccaagctcaacatcaaatactggaatcatgtcagtagattctttccgagtagattcagtcaatttgccagtttgcatggttggcataaagtctgcatcacgagtacctttcatagtacgaatagtaccatcagacttttcaaaaactacgtttgaaacacccatggacaatttagttttcaaaatttcacgaattgctactttctgctcagttgtcagtttcatttatttacctattacagttttaatatgagttgttccacgttctttaagggtggaaagtaatttttggcatttttctaaatcagatttccaactatatggtctatcaatacaaacccaatttgtcttataatactgtttccatttagagaaaaaatatttcttatactctacagcaaatgagatgttctcgttagaataagaactaattgctgtgagttttaccaaacgaaatttcattattcaccacagaattcgttgatattttcccagtttaacttattcaagtttttcttaggaacattaaacacttcaatacctgcatttcgcagaatatcatcccaaccgggtttatttttgtcgtatgtttcacaataaaccagctttttaataccagattgagctatcgcttttgcgcaatctggacaaggagaaagtgttacatacatagtagcaccttcaatagaagaaccatttcgtgcagcaaacaaaattgcatttagttcagcatgaatttcatttttagatgaccattccgagtgagcactacgatgttctttcgccaaaacaaaacgatcagttgaaccaaatgatacgcattcaggcttatgaccttgaatgatagcatgtttaggcttattcaacaaccatccttgctcagcagcataatcacaacagttcacaccccctgcgggtgaaccattatacccagtagaaataatacgtccattcttttcaattactgctcctaccttccaggagcaacattttgattcctgcgatactaaatatgcaatttgaagtactgtactcgctttcatttcataatcaccagataagcagatttagcagtttcaacacgataaatttcgtgacgaagtttagttatacttttaataacagaactaattatattctgcccatctttaaagcggtttttcttatcaataaaaactgcgccagtcatctttttgtgaagctcaactggatacttcgtcacaataatagcatcatacacagaaggatgaatactattcaccagagtatcattcattaaagttattctaatgaactgtgctgtttcagaatcaagcgctctatgatcgccagtatcattttcaagacaattatcaattatatcagttaaattcatcatagtacgccatacaccctttgtgcttcaactaatccatcaaaatccagtttaagatgcgatatttgatcgccatcacctggattcacaattactaatactgaacgaggagtttcggtaataacacgaaccgatgtttcaggaaatcgttcagaaaccttatttactaattcctgcgcaaatagtttaactttttcttggaattcctttaacagtaatcggtttttcacttagcaacattttgttttcctcatttgttttggtagagctataatatcacaactctaccgtaaagtaaaccattaaatcgctttgaattccgcagtttgagattcaaagcgaatatcgcctttgataacaagctcagcatcaagaccaaatacgacaatgatatgcgcggaacctggatacagtgtaatggcaatagaatccacctggtctggaagcaaagtgttcaatacatgagtcacttgagcatggattcgaagctcagctgcgttatcaagtttttcaaacatattattagcgataatttggctaaacactacttctacgattttagagtaagtcggaaacatatttacctcacataattttcttcaagccaatcaataacatccaacgcattatcaaaagttgaaccatctactctgtcttctgtttcataatcaagaacatctaggcctactcttccgtcaacaataggccatagacaaaatagatatttcttttctttttcaattttatcacaaagacgataaatcttttctaggttattcataagttttccatggtaaaggcagtttagttttctttactactagttcaacatcgggattcttttctcttaatttaagacattcctcccatgctctattttcactagtaaatacacaaaattgcccattactagtaccaactaaaccgctatttacaataacaatagcccaagtttcatggtgccaagccattaaaaatctcccgaagcgacttgccagcattcaacaccgatacgacgccacatttcaactacttgagttcggtcatcaatagctaatttcacgtcaaaatgcggtgcaatgtgtttccagaaaatttcttctttaactacatcgtctttacgggtatcgccttgttcgcgctgacattgcataactaatggaacgccagcaatgtcctcaacccatttacgggtcatacgataatatttcgttgggtcttctttagttccactttcacgacctgaaacgactacgatttgataacccataagagcatacatcttagacagttcaacaaccataggattgataacatcggtatcgcatttttcaaggtcataaggaccacgaccattcattttagctagtgtaccatcaacatcaaaaataactgcttttggtttaccaggagtcccattatatactggaagaccgagatactctcgcatgcttttatacattgaacgtaaaacatcaattggtactgctttagttccgcgttttgagttacgtttaaccaattcagtccaaggaacatcaaacactttatgttcaactttccagccgtattctttggcaaaagtttcccatgctaggcgacgttcaggattcaggttagtatctgaaatgattactcccttaacagaatcgccaccgtacagaatacttttagctgtatcaaactgcataccagttacgataccttctttctttttggtatacttgtactcatcgcgttcttcatgcgccataatagattggcgatagtcatcacgattgatattataaaacccgggattcttagcaataaattcacgagcccaagtactcttaccagaaccaggacagccaatagtcaaaataatctttttcatttattttttctcaactaatgattgaatataatcatgtaggtctttagatgctttaccccacttattttgatattcatttttgagattagcacgtgattgagctaataaaacatcattagttggaggtaaagattctaaccgctgaatctggcgtccataaatcattgcagccatctcggattcataaatcaatcctttgagatgttcaaattgatgccatgaaatcatttacatttatcctcttttagctcttgacgataataacatrtcatagttttctggtcatgtacatatcgttttacatcattaagccaaatacgaaattcctgagaatcttcaaatggcataccgacccaggctttaccatcaataactttaacttgccaagatagtttagcttcatcatatgactttatttgcacaggccaattaggatgaactgtttctttctttacttctagaggctttgtcgaacaaccaactagaagaccaatagataatattactgctgatagtttaatcatttagaaaggtcctggatgtcttctgcgaacttgttgaaggagttgttgatttgtttttcaaccaatcctggcttatgagccaccacatccgccttctttgcatctttgcgcagtttttcattttcacgctcaatagcagcaattgcctcacgatttttattattcatcgcatcaatataattatactgaattcgcaaattatttaatgctaaggcgttttcattggccgtttttgtaatttcagtaactgatgtttctaatctttctaccttatgttttaaaataatagatgttccgccaaatgctattacaagtaatagcaatccagctgtaaaattacttacatgcataaagttttaataacctctacaatatcgtcttgagaaagaccgttaattaaaatatgatgttcagctggagatttagaaattttaaagcatgcctcaacatcttctgccatatccgatgcactacgatttggattactaataccaaggcgatgttttcccgtcaaaggattaacgatgatatagcatttgcagctattgatatgaacattgggttgagtctgattaatgaacacttcacaatcatacttagcaagttgattttctaaaaagactttcatctcttcaaccgcatcaggaagcatatcacgggcttgctcaagacgacgatttcgatattctttaatggtcgttttccgcttgacttgcttagctaaatctttcttaagaccagtgatatatccaactcgacgatttcctttgaatacagaaatcccatctgtagtatcaccgtatgcttcaacgaccatttcagtagtaataagttgtaaatccatcataaagtcctcatgttatgtcagtaagactactataacacaacacgagggacttgtaaacaacttagtatccttctgggataaattttttataatttttcaaaaaattctgttcgatttcacacatgaccttttcttgactatcgtacccctggtataagctcatgatgataccgaacaggtgatccattccagcacctttagcaacaccttgtgcttccattgcataagtctttctatccttaccgcaatgcttattatgacagtcaagaactaaaaacagagctcggtctaagtacttcagataagtcgtttcaaatgcttcaatttttctgtatgaatattcatcgtcagcatacattgctttaagatcatctgatgcaccatcaataatagtcttaaacaatttttctggattatctaatgaactttttgtactatgaagagacacgtaccagtcagacttaattttaaaatgagaaccatctttcatcacagcaacatagccttcgatgttttctgcatttttagcttcttctatccatttagggctatcgatttcgtatcgttcaactagatacggacgaagagtagcatctttataaatatcatcgtatgaaatgtattcacccgtttcgttttcacgaacattcagtaaaataattttcatctcttgataagcaagaacgattctattcgtcggggcaacgaattcgaagttagcagtaaatccatcttcagctaattctttaagtctatcacgcaaccgatggtgattaatattcatcaaaattccattagccattaaagcctgctcagatttgattgaaccctttgatttgaacagaatttcatcaccgtctaaataagttgataccaaagacccgtcttcttttgttagaatataatcaacatcgtttaaatcgatattcatcgtgaacggattttcattcaagttaaaaaacttttccataggacgagaagcaattcttactggtttttctccatccatttcaaacataattccacgacattctagtgcatctggaagtaaccaatcagaataagatgcataattatatgagaaaattctgtaagttcttccagatgcacttacatcatctgagtaaaaaaacttacgctgcgaatccttacatagttccattaaattgttaaaaagttcttgcattgtgtatcctcttttgtgttttgaatatagtaccacactccatgtggaagcatcattttttcttgtgttgaatattccaaggcgggttaaacagtttaatgaatagaggctcctctaagtcaatcgttgcgattgtcattgtacctaactcatttgtcatagaaagattaaaacattggcgggcgtaaaattcaactttgcttccttcctttagcgcagaatgaattaatgcagatttagtagaatcagacgttttgtctttgcggttaatagcagttctataatagtttattcttttacgtaaatttttagtttttccaatataaacaagctcatcatttatagcaatagcataaattacgttatacttgtttggaatagataattgttttatacttccattgtcgtctaattctagctcagtatatttaataaatgaatattctgttgcaatttctttcataataaaatgggccttgcggcccactccttaaaagtattttttaaaactcatcataactttatcatcaacatcattatcaatctgtgcaacaaggtaagatgacagttctacttcttgcggcgcggattgaacattatcagaattaagatattcacgaatccaaggatatggatgtttaaccggagcatcggtaattgggcatggaagaccgcactgtttcatacgagatacagttaagtaatcaataaagctccacatgctatttgtatttaatccaggaacatcaccatctttaaataaatgaactgcccagtctttttcctggcggttaacttccatgaaaatatcaactgcttcttgttcacactcttgggcaattttaacccattcatcaccatcagtaccggattgaagttgacgaataatatattgggtgcctttaaggtgaagctgttcatcacgtgcaatgaacttcataatcttggcattaccttccatgatttccatgttcttatggaagttaaaggtaccgtatgttcgattgagctcgctaattctcaacccgttctcttatgaactgctgcatgttaccatgcagtccagactatatcacaatcccggagggattctccccatttcgagtcgcttgaccctacgtccgaagactagtcgttgaaccttgttttgcaattgtcgccgtgccatcgtttatatgttgatggtgataaatctttcttatcacaatatggacaattcaattgaattctatttggatttaatttacatctgtcattatgttttagataaaatccaggacctttaccaatgtgaccacagaaatcacatgtgatttctttttgtgcaggatgggtcccgttcttaaccatttctaatgtttttgctgatgttcttttcttatgttcttccatcagaatctttaggtataggtccgttagcatctatccaaatttttctatagttcaattgtgcatcctcttaaaagtataatcatatttatattatactaattaaaggtgcaagcaaaaccttggctgctagttttccataaaggactttccagcaattaaaggagttttcgataaacgttaccgtttaaaggcgcattttacgcaaaagatacataaaaacgaatagcttctaaggcattaataacgtgcaaacagaggtaaagagatttcatcagatctcgtttagctctttgctcaacgtctttatcagctagaattagaccttggtctttataatattcaaccatgtctttagcattttcccattcacgggttttaaccaagacatcatcgtaatatcgcccaatggactcagcacgtttcataatagcttcatctaatacaatctcatcaaacaccttcgatggatcagtataaagatttcgcatgatatgagtatatgaacgactgtgaatagtttcactaaaagtccatgtagcaacccatgtatcaaggcttgggtctgaaattaatgacataagtacagcagatggcgcacgaccctgaatgctatccaaaagtgattgatacttcaggttgttagtaaaaatattttgctgatactgaggaagcttattaaattgcgcagcatccatcattaagtttacttcttcaggacgccaaaaaaaactgatctgccgctcaatgagttcttcaaatactcgatgtcgttgaatatcatatcgagctaaacctaatccagaaccaaagaacatcggttcattcaaaacatcaactggatttgtattaaaaactgtactcatttagaatccttaaatttacatttatcataatgccatcttaaagcattgcctttattaactttcttaccacagtgcgggcaaggtggatactctgctccttgtttagttctcaaagaaatcaaatcacgggtttctttagtatgaggaacatctcttgtcggagaaattgtaccatacatcggattaagcacgccgactttagcagcagctatattctttttagcttcttcagttttaggctttctcatctttttcttaggtttcttcgcttttagcccttcctttggttgccgccgatattttgtgtttggtttcttctgttaaacgcagacctgttttcgctttttgacattttcagtctcgtttcttcagaaatgaccgtacctttgcgaaattgcgaatttaacttctttgcatgggcatatattttagaatgaactttataatgacgtttcttagttcctttcatattgcacatcatgaaaaatgcgaatataacagattttaccggataaatctttgataaaatagcatgcgctataaaatgctctctagctgttaattcaactaaattttctttatcatcagaacctcccatgcatctagggattatatgatgtgtctctttatattcggataaaggttcccgagcctgagctcgggaaattaggtcgttatagattttttgataattcattacaatttacacgctgcacaatcatcggctttaggagtttctatttcataatcatcagtaccagaaccatcacgggtattatgataatagaaatttttaatgccataataccatccgtatagcatgtcatcaatcattattgacattggaacctttccttttggaaaaatctgcgggtcataatatgtattcgctgaagctgattgacatacccatttcagcataatagctacctgcgtaagataaggtttattacctttcttagctaatttccatgtataatcatagaggtctatgttatgctcaatattgggcacgacttgattaaaggaaccctcttttgattctttaacacttaccggtccacgtggaggctcgtagccgtttgtactgttagaaacttgggaagatgactcacatggcataagtgctgataatgtgctattacggatgccaaatagcttaaggtcttcccgcagcgccgaccagtcacaaacgtattttggagctgcgatttggtcaatctttttattgtaccagtcgataggtaattcgcctcgagaccaacgagtgtctgaataatattccgaaggtcctttttctttggcgagcttaatggatgctttaatgagtccatactgtaatctctcaaatagttcatgtgttaaatcgttagcatcttcataagaagcaaagttacttgccagccaagctgcatagttggtaacacctacaccgaggttacgacgctttttagctttttctgcttcaggaaccggatatccttggtaatccaacagattatcaagagcacgaacttgaacttctgccaattcattaattttatcttggtcttgccagtcaaaattatctaatacgaatgcagagagagtacacaatccaatttcagcatcagggctattcacatcatttgttggaatagcaatttcacagcacaagttactctgacgaataggtgccttttcacgaataaacggagtatagttattcgtattatcaatgaactgcacataaatccttgctgttcctgaacgttcagtcatgagcaattcaaatagttcacgggctttaatacgctttttacgaatattagggtctttttctgctgcttcgtataattcacggaaacggtcttggtctttaaaataagaataataaagctcgccacccatttcatgcggactgaacaaagtaatgtaatcgttttttccaaaacgttccatcatcaaatcattcagttgaacaccataatccatatgacgaatgcggttttcttctacgcctttgttatttttcaaaacgagaagattttcaacttccaaatgccaaataggataataagcagtagcagcgccgccacgaattccaccctgtgaacatgatttaactgcagtctgaaaatgtttccaaaaaggaataacaccagtatggcgtacttcacccatgccaatcttagaaccttcggcacgaatcataccaacgttaataccaattccagcgcgtttagagatatattcaacaattgaagcagaagccttattgatagacttcaatgaatctcctgcctcaataacaacacatgaactaaactgtcgagtcggagtacgacaaccagccataataggagttggcaatgaaatctgtcgagttgatactgcttcataaaaacggataacatgttttaatctatcaacaggttcatcttgatgcagtgccattccaatagtcataaatgcaaactgtggagtttcataaatttgaccagtggttttatctttaactagatatttttcttttaattgcattgccccggaataagtaaattccatatcccgttcgtgcttaatttttgattctaaaaatgtaatttcttctgctgaatattttgacaataattcagggtcgtatttacctgcatttacacaataagaaatatggtcaataaatgaacgtggttcatactgcccataaacatgcttacgaagagcaaacattaaacagcgtgcagctacatattgataatcaggttcttcaaccgaaatagaattcgcagcagccttaatgacaatagtctgaatatcatcagttgtcattccatcacggagataagatttaatattttcatataattcataaggatctactgatgttccttcagctgcccaagataaaactttaataattttttgtgggtcaaagctctgagaaacaccactacttttgataacattaattaattgcataagtcctcaacttgaaaatcgtctttaaacaatcggttaactatatgagctattatatcaccatgacacggctttggtttacatgtgcatcctagcctcattccacgtaaaggctctaaatgtgctttagttatttctccggatttaattcgacgtataaaatcttttttgaataattcaatggcagcctcccggctgccagcatctttaccgacgtaatttccccaaaatgtaccgcggtgaatattaacatcaaagtcggatttatatttattcactacccggcatagacggcccacgctggaataattcgtcatattgtttttccgttaaaacagtaatatcgtagtaacagtcagaagaagttttaactgtggaaattttattatcaaaatactcacgagtcattttatgagtatagtattttttaccataaatggtaataggctgttctggtcctggaacttctaactcgcttgggttaggaagtgtaaaaagaactacaccagaagtatctttaaatcgtaaaatcatatatcctcgcaataataaaattacaccgccatctttcctttaataggagggtgtgatacatagttgttaagaacgaaatctttaggcctaagtttaagaacatattttaattgttctttagtagaaagatatcggaatttataaggtagaccacttattaccagctcacaaagctctttaggttcacgcctcaaaatttctttacattgttctacgtgattcatatagatatgagtattaccaccagaaaatatcaaatcccctggaataagattacacatcttagctacaatatgaactaacgtagcatatgacgcaatattaaacggtagcattatgttcagataaggtcgttaatcttaccccggaattatatccagctgcatgtcaccatgcagagcagactatatctccaacttgttaaagcaagttgtctatcgtttcgagtcacttgaccctactccccaaagggatagtcgttaggcatttatgtagaaccaattccatttatcagattttacacgataagtaactaatccagacgaaattttaaaatgtctagctgcatctgctgcacaatcaaaaataaccccatcacatgaaatctttttaatattactaggctttttacctttcatcttttctgatattttagatttagttatgtctgaatgcttatgattaaagaatgaattattttcacctgaacgatttctgcatttactacaagtataagcagaagtttgtatgcgaacaccgcacttacaaaacttatgggtttctggattccaacgcccgtttttacttccgggtttactgtaaagagctttccgaccatcaggtccaagtttaagcatcttagctttaacagtttcagaacgtttcttaataatttcttcttttaatggatgcgtagaacatgtatcaccaaacgttgcatcagcaatattgtatccattaattttagaattaagctctttaatccaaaaattttctcgttcaataatcaaatctttctcatatggaatttcttccaaaatagaacattcaaacacattaccatgtttgttaaaagacctctgaagttttatagaagaatggcatcctttttctaaatctttaaaatgcctcttccatctcttttcaaaatctttagcacttcctacatatactttattgtttaaagtatttttaatctgataaattccgcttttcataaatacctctttaaatatagaagtatttattaaagggcaagtcctacaatttagcacgggattgtctactagagaggttccccgtttagatagattacaagtataagtcaccttatactcaggcctcaattaacccaagaaaacatctactgagcgttgataccactgcaaatccaaatagccattacgcacattaaactgatagaacatatgacaaggcggtaatgccatatatttaagttcagctggattccatgcagaaacaatttgacgcctatcatttggcagttttttaatacgatcaataacttctataatttggtctacaccaccaaaatcacgccactgttttccataaattggaccaagttcaccgctatggtatcctaaatcttttgcttgattttcgtaattttcatcccagactgttttgccttggattaacgaatcgtgttgaattaatcgtaaatcattgacatttgtgcttcctgataaaaaccatattagctcagcaatgcaagctttccaggcgagcttcttagttgttaccgcaggaaaacctttagttaaatcccagcgtaatttagatccgaacagagcaattgttcctgtgcctgtacgatcatcggtttcataaccattttcaaaaatgtctttaattaaatcttggtattgtttcatttatatactgattccgtaagggttgttacttcatctattttataccaatgcgtttcaaccatttcacgcttgcttatatcatcaagaaaacttgcgtctaattgaactgttgaattaacacgatgccttttaacgatgcgagaaacaactacttcatctgcataaggtaatgcagcatataacagagcaggcccgccaattacacttactttagaattctgatcaagcatagtttcgaatggtgcattagggcttgacacttgaatttcgccgccagaaatgtaagttatatattgctcccaagtaatatagaaatgtgctaaatcgccgtctttagttacaggataatcacgcgcaaggtcacacaccacaatatggctacgaccaggaagtaatgtaggcaatgactggaacgttttagcacccataatcataattgtgccttcagtacgagctttaaaattctggaggtcctttttaactcgtccccatggtaaaccatcacctaaaccgaatgctaattcattaaagccgtcgaccgttttagttggagaataacggaataccaatttaatcattacgtaaatcctattttaattgaaaacgaatgcttacttggataatttcaatgacatacataatattttcctcaaacagactttttcacaattttccaatcagctttaaactgctcgacgtcagaatggtaaatccaaaatcctgcgctttctccgtcttcataaagaggacatccatcgcattcatcttcccatcccatatcacgtaaaagatgttcagctttttcaacaagttcagaatctttaccgatgatattaaaataccatttacctctaacttctgaatctttgatgctctggcgttgtaatctcattttattctccttagcaagctttaatcaaaagatataaacagaccaacataactgctgccataatataaggtgcgaacattttcttttctccattagttttgatagggtaatagtatcacactactaccctgatgtaaactactttttgaaagtttttcgcaaaagttcaatgatttcatctacattgttttcgtcaacaatgcagtgaatttttgttacgccagaaaccttgtctttgacttcatcttcttcagaagtcggttctttatattcgcggaaacaataaaactcttcttcactaagttcaaaataatcatcacccataccatcatcattatagatttcaccattagcacaaatgatttcggttacataatcaaagccatctaaacttgatattgatttaacttcaaaccaaccgccattttcttgaatgatgccgaccatactagcatttgatgaactaatatcaatgaaagatttaatacgatgtggatttaactcgtattttttgccgatttccattttgattttcctcattttaataggggcttgatagccccttgataattattgttcaatcagtcccatgtaaaattctgcgtcttcagaatccatgccatcacaatattcattagccataaagcgggtgaggtcttcaagaggaccttcaataacgatttgaatactccaaaacttagaatcttgcacgcttgtgatactaagttcaggataacgattacgaataatctcttcaatatattcaaaatcaacgatgtcaatatcaactttagccatattattttcctctttaattattagcagtattgccgatagttgtatagtaccataaagctttatgcttgtaaaccgttttgtgaaaaaatttttaaaataaaaaaggggacctctagggtccccaattaattagtaatataatctattaaaggtcattcaaaaggtcatccaggtccgtgtcatcagcactagatgaactaccagagcttgagctcataaaatcatcttcagtttttgtattgaagtcatcaacattgaatgcatccaaatcatcagccactttatcagctttcttagcagcagttgcagcagcaccgcccatcacagcagttcccataacttgaccgaatttagtattaagttcttcaaacgatttgaatttatctttagaagtcatttcagaaaggtcaaccatttgttcgaacagttctttctggaaagattcatcgtcaatgtttggaatcgcagattgattcaggaatttagattcatcgtagttactaaatccagaaacttgtttaactttcagtacaaagttagcaccttcccacggacaagttacatcaactggagtttcacccatttcaacatcaaccgcaatcattgcattgattttatcccagattttcttaccaaagcggtatttaaatactttaccttcgttttctggagcagctgggtcttttactacaagaatgttagcccagtaagaagttttacgtttaacaagactgtactctttattgtcagtgttgtatagatcatttttactgatgtattgacatactgggcaagaatcgtaatcaccatgggtagatgaacatgtttcaatataccatttaccatttttcttgaaaccgtgatttacaagaattgcgaatggtgcttgttcatcatttttagacggaagaaaacgaattactgcttgaccgttacccgcattatcgagtttcagtttccactcgcctttatcttcagaagaaaaacctttattgccattcagtttagccatttgtgcagcgagttcagcagtagatttacgtttaaacatttttatttcctttttaatttaatttaattaacagttggtgctatgacactttacctcatagctggcataattcgcaatactctgggtcttcgagaggtatccaacctgagttgaaatactttaccatcgatttagcagttgtatcagttatatttatattacctttaactcttcgccatccaggagttttaccgtacagattagaggataataataacacataattctcgtaagcaatatgagataatttccaagactctatattagctcgtgatgttttccaaggtctaaaatcgtcacggttcatataattagccaatctcatatgctctctaacttccgggtctttggctggatgagtttcaccactcacaccaaatccaccaccagcatataccagattaaaatagtctggattatctctggcatttacttcaagttggtattttcgttctgcttcaataacatccaagtcatcatcaatttgaattattttaacgctcggtttttgaagcattagcgcattcaaaaatcttttttgtttacatgagctccagtattcctttccggaagagtcatatattattccgttctcaaatgagcaatttaatttactaccgatatagtagtatggcggagtcttatttttgacacggtcttcaaatgtaaaccaatatactatattcatatcaatacttgcaagatttcacagtttcaatgaaaacatttttagctttctgtgaatcaatatttaaaatttttctataagcctttaactttatagaataattattccagactaaattatcagtctgttcatcatgtttatcaattatatttaaaaacgaatcaagcaagataaacgtctcaaacgaaattatgttcgattgcagaagtttaaaaatataacttgattgaacttttggattatactcaaaaatttctttaaaagcagaaacttcaacttttttactaaaataataaatgttgcgaatatcttcttcaaacttaaatttaatttgctttaagcgtccgatatattcacgataaaacacaagtgcatcagcgtcagagatgtcaccaatccaagcatcttggttagcaaccaaattgcttataaagattaaagcaagttcctttaatttatatttttctgataacttctggaaaaaatacttatcccttcgcttttgataagcggcatcagacacccgcatgcaccaattatacttaattacatcatactttccattcatatgttgttttatcattaagtataatttataaactgatttaccatcaatgtatctttcaccaccagcaggcatgcggagtttaatcatagtagaaaatctaatgtattagttttttcacaacgaacaacagaaggacgtaaaagattttcgtcaatagcttctgactgaattttttcaattatacccgaaggaataaatttagcaaattgagtttcaggaatagaattttcttctaagaatgctgttgtagcttcaagataactcattccaaactcttctaccattttttcaataataaatccattttcttggcggtcaagaagctttgctatttcatccttttctttcttaattgaaagttctttttctgaaagaccggtctcatcgaccggacgaatatcatttagagaaaactgtgtcataaagttcaactacctcttcagtttcagcttcaaacacatcacggttatctttatgatacaaagctaatagacgattaaacatcttaccatcaacgccaagttcatctttagcacgaattcgaatatctttaatcagttcattataaccggaaattttcagtttatgatcagatgcttctttaataaatttagccaagtcttcgccatggatagcttcatcaaattcaaccatttcttttttagccattattcacctcaaaattcattaatgctattagttaatttagaaagacccgcttttacaaaatatgaataaattttgccacgcggtggtaatttatatgaattatagtaattcacaatgtttgaagcaatattatcaggaatataatcaaaatcaattagaactaaattttctttataacgattatattcagattcagtgagaagcaccttagcttgctcacggtcattagcaatagcttcaacgattgaagttttcattgaaggagttcgttcaccttcaactctggtaaaccaaaagtcagatcgtactttaactgaagcaacgttatcctttttgtcgcctttaaggattttagtcatacagtcaatttcagcagaaccgcttttaattttaacccatttcttatgcatcggagaccattgcttaacatttggatatttgtgaagctgagtaaagtcaccatctgacgaaatgattaaaatcttatgtccttctaaagagaactttttaacaagaacagcaatgtggtcatctgcttcatacttatcaatatccataacaatgtatggcatataagctttcaattcatctataactttatggctggattcaaaataaccttcccagtcccaagtagattcttctcgtgcttttccacggtttttcttataataataagcgaaatcacgacgccaatatccagatttcgcgttatcaatacacagtacaattttagtgtatccaagcgtttttgcttttttgacattaaacttaattgagttcaatatcaaatgacgaaccattgataaattaattttttctttatctgggaagtttaccaaagcagttgaaagcgcaatttgactaaagtcaattaagcagattccttctttgtaatcttcatccaacatcatttctaaatccatatgaacctcgttcaattagtgagatttctattatataccatccaaatcttaaagtaaacaagtataaatacttattattgaaaacacaataggagcccgggagaatggccgagattaaaagagaattcagagcagaagatggtctggacgcaggtggtgataaaataatcaacgtagctttagctgatcgtaccgtaggaactgacggtgttaacgttgattacttaattcaagaaaacacagttcaacagtatgatccaactcgtggatatttaaaagattttgtaatcatttatgataaccgcttttgggctgctataaatgatattccaaaaccagcaggagcttttaatagcggacgctggagagcattacgtaccgatgctaactggattacggtttcatctggttcatatcaattaaaatctggtgaagcaatttcggttaacaccgcagctggaaatgacatcacgtttactttaccatcttctccaattgatggtgatactatcgttctccaagatattggaggaaaacctggagttaaccaagttttaattgtagctccagtacaaagtattgtaaactttagaggtgaacaggtacgttcagtactaatgactcatccaaagtcacagctagttttaatttttagtaatcgtctgtggcaaatgtatgttgctgattatagtagagaagctatagttgtaacaccagcgaatacttatcaagcgcaatccaacgattttatcgtacgtagatttacttctgctgcaccaattaatgtcaaacttccaagatttgctaatcatggcgatattattaatttcgtcgatttagataaactaaatccgctttatcatacaattgttactacatacgatgaaacgacttcagtacaagaagttggaactcattccattgaaggccgtacatcgattgacggtttcttgatgtttgatgataatgagaaattatggagactgtttgacggggatagtaaagcgcgtttacgtatcataacgactaattcaaacattcgtccaaatgaagaagttatggtatttggtgcgaataacggaacaactcaaacaattgagcttaagcttccaactaatatttctgttggtgatactgttaaaatttccatgaattacatgagaaaaggacaaacagttaaaatcaaagctgctgatgaagataaaattgcttcttcagttcaattgctgcaattcccaaaacgctcagaatatccacctgaagctgaatgggttacagttcaagaattagtttttaacgatgaaactaattatgttccagttttggagcttgcttacatagaagattctgatggaaaatattgggttgtacagcaaaacgttccaactgtagaaagagtagattctttaaatgattctactagagcaagattaggcgtaattgctttagctacacaagctcaagctaatgtcgatttagaaaattctccacaaaaagaattagcaattactccagaaacgttagctaatcgtactgctacagaaactcgcagaggtattgcaagaatagcaactactgctcaagtgaatcagaacaccacattctcttttgctgatgatattatcatcactcctaaaaagctgaatgaaagaactgctacagaaactcgtagaggtgtcgcagaaattgctacgcagcaagaaactaatgcaggaaccgatgatactacaatcatcactcctaaaaagcttcaagctcgtcaaggttctgaatcattatctggtattgtaacctttgtatctactgcaggtgctactccagcttctagccgtgaattaaatggtacgaatgtttataataaaaacactgataatttagttgtttcacctaaagctttggatcagtataaagctactccaacacagcaaggtgcagtaattttagcagttgaaagtgaagtaattgctggacaaagtcagcaaggatgggcaaatgctgttgtaacgccagaaacgttacataaaaagacatcaactgatggaagaattggtttaattgaaattgctacgcaaagtgaagttaatacaggaactgattatactcgtgcagtcactcctaaaactttaaatgaccgtagagcaactgaaagtttaagtggtatagctgaaattgctacacaagttgaattcgacgcaggcgtcgacgatactcgtatctctacaccattaaaaattaaaaccagatttaatagtactgatcgtacttctgttgttgctctatctggattagttgaatcaggaactctctgggaccattatacacttaatattcttgaagcaaatgagacacaacgtggtacacttcgtgtagctacgcaggtcgaagctgctgcgggaacattagataatgttttaataactcctaaaaagcttttaggtactaaatctactgaagcgcaagagggtgttattaaagttgcaactcagtctgaaactgtgactggaacgtcagcaaatactgctgtatctccaaaaaatttaaaatggattgcgcagagtgaacctacttgggcagctactactgcaataagaggttttgttaaaacttcatctggttcaattacattcgttggtaatgatacagtcggttctacccaagatttagaactgtatgagaaaaatagctatgcggtatcaccatatgaattaaaccgtgtattagcaaattatttgccactaaaagcaaaagctgctgatacaaatttattggatggtctagattcatctcagttcattcgtagggatattgcacagacggttaatggttcactaaccttaacccaacaaacgaatctgagtgcccctcttgtatcatctagtactggtgaatttggtggttcattggccgctaatagaacatttaccatccgtaatacaggagccccgactagtatcgttttcgaaaaaggtcctgcatccggggcaaatcctgcacagtcaatgagtattcgtgtatggggtaaccaatttggcggcggtagtgatacgacccgttcgacagtgtttgaagttggcgatgacacatctcatcacttttattctcaacgtaataaagacggtaatatagcgtttaacattaatggtactgtaatgccaataaacattaatgcttccggtttgatgaatgtgaatggcactgcaacattcggtcgttcagttacagccaatggtgaattcatcagcaagtctgcaaatgcttttagagcaataaacggtgattacggattctttattcgtaatgatgcctctaatacctattttttgctcactgcagccggtgatcagactggtggttttaatggattacgcccattattaattaataatcaatccggtcagattacaattggtgaaggcttaatcattgccaaaggtgttactataaattcaggcggtttaactgttaactcgagaattcgttctcagggtactaaaacatctgatttatatacccgtgcgccaacatctgatactgtaggattctggtcaatcgatattaatgattcagccacttataaccagttcccgggttattttaaaatggttgaaaaaactaatgaagtgactgggcttccatacttagaacgtggcgaagaagttaaatctcctggtacactgactcagtttggtaacacacttgattcgctttaccaagattggattacttatccaacgacgccagaagcgcgtaccactcgctggacacgtacatggcagaaaaccaaaaactcttggtcaagttttgttcaggtatttgacggaggtaaccctcctcaaccatctgatatcggtgctttaccatctgataatgctacaatggggaatcttactattcgtgatttcttgcgaattggtaatgttcgcattgttcctgacccagtgaataaaacggttaaatttgaatgggttgaataagaggtattatggaaaaatttatggccgagtttggacaaggatatgtccaaacgccatttttatcggaaagtaattcagtaagatataaaataagtatagcgggttcttgcccgctttctacagcaggaccatcatatgttaaatttcaggataatcctgtaggaagtcaaacatttagcgcaggcctccatttaagagtttttgacccttccaccggagcattagttgatagtaagtcatatgccttttcgacttcaaatgatactacatcagctgcttttgttagtttcatgaattctttgacgaataatcgaattgttgctatattaactagtggaaaggttaattttcctcctgaagtagtatcttggttaagaaccgccggaacgtctgcctttccatctgattctatattgtcaagatttgacgtatcatatgctgctttttatacttcttctaaaagagctatcgcattagagcatgttaaactgagtaatagaaaaagcacagatgattatcaaactattttagatgttgtatttgacagtttagaagatgtaggggctaccgggtttccaagaggaacgtatgaaagtgttgagcaattcatgtcggcagttggtggaactaatgacgaaattgcgagattgccaacttcagctgctataagtaaattatctgattataatttaattcctggagatgttctttatcttaaagctcagttatatgctgatgctgatttacttgctcttggaactacaaatatatctatccgtttttataatgcatctaacggatatatttcttcaacacaagctgaatttactgggcaagctgggtcatgggaattaaaggaagattatgtagttgttccagaaaacgcagtaggatttacgatatacgcacagagaactgcacaagctggccaaggtggcatgagaaatttaagcttttctgaagtatcaagaaatggcggcatttcgaaacctgctgaatttggcgtcaatggtattcgtgttaattatatctgcgaatccgcttcacccccggatataatggtacttcctacgcaagcatcgtctaaaactggtaaagtgtttgggcaagaatttagagaagtttaaattgagggacccttcgggttccctttttctttataaatactattcaaataaaggggcatacaatggctgatttaaaagtaggttcaacaactggaggctctgtcatttggcatcaaggaaattttccattgaatccagccggtgacgatgtactctataaatcatttaaaatatattcagaatataacaaaccacaagctgctgataacgatttcgtttctaaagctaatggtggtacttatgcatcaaaggtaacatttaacgctggcattcaagtcccatatgctccaaacatcatgagcccatgcgggatttatgggggtaacggtgatggtgctacttttgataaagcaaatatcgatattgtttcatggtatggcgtaggatttaaatcgtcatttggttcaacaggccgaactgttgtaattaatacacgcaatggtgatattaacacaaaaggtgttgtgtcggcagctggtcaagtaagaagtggtgcggctgctcctatagcagcgaatgaccttactagaaaggactatgttgatggagcaataaatactgttactgcaaatgcaaactctagggtgctacggtctggtgacaccatgacaggtaatttaacagcgccaaactttttctcgcagaatcctgcatctcaaccctcacacgttccacgatttgaccaaatcgtaattaaggattctgttcaagatttcggctattattaagaggacttatggctactttaaaacaaatacaatttaaaagaagcaaaatcgcaggaacacgtcctgctgcttcagtattagccgaaggtgaattggctataaacttaaaagatagaacaatttttactaaagatgattcaggaaatatcatcgatctaggttttgctaaaggcgggcaagttgatggcaacgttactattaacggacttttgagattaaatggcgattatgtacaaacaggtggaatgactgtaaacggacccattggttctactgatggcgtcactggaaaaattttcagatctacacagggttcattttatgcaagagcaacaaacgatacttcaaatgcccatttatggtttgaaaatgccgatggcactgaacgtggcgttatatatgctcgccctcaaactacaactgacggtgaaatacgccttagggttagacaaggaacaggaagcactgccaacagtgaattctatttccgctctataaatggaggcgaatttcaggctaaccgtattttagcatcagattcgttagtaacaaaacgcattgcggttgataccgttattcatgatgccaaagcatttggacaatatgattctcactctttggttaattatgtttatcctggaaccggtgaaacaaatggtgtaaactatcttcgtaaagttcgcgctaagtccggtggtacaatttatcatgaaattgttactgcacaaacaggcctggctgatgaagtttcttggtggtctggtgatacaccagtatttaaactatacggtattcgtgacgatggcagaatgattatccgtaatagccttgcattaggtacattcactacaaatttcccgtctagtgattatggcaacgtcggtgtaatgggcgataagtatcttgttctcggcgacactgtaactggcttgtcatacaaaaaaactggtgtatttgatctagttggcggtggatattctgttgcttctattactcctgacagtttccgtagtactcgtaaaggtatatttggtcgttctgaggaccaaggcgcaacttggataatgcctggtacaaatgctgctctcttgtctgttcaaacacaagctgataataacaatgctggagacggacaaacccatatcgggtacaatgctggcggtaaaatgaaccactatttccgtggtacaggtcagatgaatatcaatacccaacaaggtatggaaattaacccgggtattttgaaattggtaactggctctaataatgtacaattttacgctgacggaactatttcttccattcaacctattaaattagataacgagatatttttaactaaatctaataatactgcgggtcttaaatttggagctcctagccaagttgatggcacaaggactatccaatggaacggtggtactcgcgaaggacagaataaaaactatgtgattattaaagcatggggtaactcatttaatgccactggtgatagatctcgcgaaacggttttccaagtatcagatagtcaaggatattatttttatgctcatcgtaaagctccaaccggcgacgaaactattggacgtattgaagctcaatttgctggggatgtttatgctaaaggtattattgccaacggaaattttagagttgttgggtcaagcgctttagccggcaatgttactatgtctaacggtttgtttgtccaaggtggttcttctattactggacaagttaaaattggcggaacagcaaacgcactgagaatttggaacgctgaatatggtgctattttccgtcgttcggaaagtaacttttatattattccaaccaatcaaaatgaaggagaaagtggagacattcacagctctttgagacctgtgagaataggattaaacgatggcatggttgggttaggaagagattcttttatagtagatcaaaataatgctttaactacgataaacagtaactctcgcattaatgccaactttagaatgcaattggggcagtcggcatacattgatgcagaatgtactgatgctgttcgcccggcgggtgcaggttcatttgcttcccagaataatgaagacgtccgtgcgccgttctatatgaatattgatagaactgatgctagtgcatatgttcctattttgaaacaacgttatgttcaaggcaatggctgctattcattagggactttaattaataatggtaatttccgagttcattaccatggcggcggagataacggttctacaggtccacagactgctgattttggatgggaatttattaaaaacggtgattttatttcacctcgcgatttaatagcaggcaaagtcagatttgatagaactggtaatatcactggtggttctggtaattttgctaacttaaacagtacaattgaatcacttaaaactgatatcatgtcgagttacccaattggtgctccgattccttggccgagtgattcagttcctgctggatttgctttgatggaaggtcagacctttgataagtccgcatatccaaagttagctgttgcatatcctagcggtgttattccagatatgcgcgggcaaactatcaagggtaaaccaagtggtcgtgctgttttgagcgctgaggcagatggtgttaaggctcatagccatagtgcatcggcttcaagtactgacttaggtactaaaaccacatcaagctttgactatggtacgaagggaactaacagtacgggtggacacactcactctggtagtggttctactagcacaaatggtgagcacagccactacatcgaggcatggaatggtactggtgtaggtggtaataagatgtcatcatatgccatatcatacagggcgggtgggagtaacactaatgcagcagggaaccacagtcacactttctcttttgggactagcagtgctggcgaccattcccactctgtaggtattggtgctcatacccacacggtagcaattggatcacatggtcatactatcactgtaaatagtacaggtaatacagaaaacacggttaaaaacattgcttttaactatatcgttcgtttagcataaggagaggggcttcggcccttctaaatatgaaaatatatcattattattttgacactaaagaattttacaaagaagaaaattacaaaccggttaaaggcctcggtcttcctgctcattcaacaattaaaaaacctttagaacctaaagaaggatacgcggttgtatttgatgaacgtactcaggattggatttatgaagaagaccatcgcggaaaacgcgcatggacttttaataaagaagaaatttttataagtgacattggaagcccggttggtataactttcgatgagcccggcgaatttgatatatggactgatgacggttggaaagaagacgaaacatataagcgagttttaattcgtaatagaaaaattgaagaattatataaagagttccaagttttaaataatatgattgaagcttctgtcgccaataaaaaggaaaaattctattataaaaaccttaagcggttctttgctcttttagaaaagcatgagcatttaggtggtgaattcccttcatggcctgaaaaagaacagaagccttggtataagcgtttattcaagcattacgtataaatatcttaaaaggagggtctatggcagcacctagaatatcattttcgccctctgatattctatttggtgttctagatcgcttgttcaaagataacgctaccgggaaggttcttgcttcccgggtagctgtcgtaattcttttgtttataatggcgattgtttggtataggggagatagtttctttgagtactataagcaatcaaagtatgaaacatacagtgaaattattgaaaaggaaagaactgcacgctttgaatctgtcgccctggaacaactccagatagttcatatatcatctgaggcagactttagtgcggtgtattctttccgccctaaaaacttaaactattttgttgatattatagcatacgaaggaaaattaccttcaacaataagtgaaaaatcacttggaggatatcctgttgataaaactatggatgaatatacagttcatttaaatggacgtcattattattccaactcaaaatttgcttttttaccaactaaaaagcctactcccgaaataaactacatgtacagttgtccatattttaatttggataatatctatgctggaacgataaccatgtactggtatagaaatgatcatataagtaatgaccgccttgaatcaatatgtgctcaggcggccagaatattaggaagggctaaataattatttgttcgtatacatctctagatatcgatatacaccctcaaaaccctcgttgaattcgtcgatgagggttttcttatcttcttgagttaattcagaaacaattttacggaatgaattttgatttaactttctaccttcatgcgttactccaatctcatttagaaatgcaataaaattagcacgattctcaacaatatcttctctggaaaatttaatcaaaatagatgcaacagtaataatttcacgaactgtatcaatgtttttattcattaactataccactcaattagttgactttgttataatatcatcagacgcttgatttgtaaactggtctgtgtaattttcttcaaaaattttttctacgaattccttgaacgattcacgttcctgagctacattatgctcgattaccttttcaagattatgactcattcgaaataatcttcaatttcataatcatggacataaatcattatagtttttaatacatcatcaatattttttcctggagctggaattacgtaaaaataccctgcttttgagaggtctttataagttccaatcaagaaatcattattctcaagatgtaactcttcaactaattcattgacaattgaatggtataggtttggcagaaacttatatagcttttctagaatatcaattttgaatgtatattgaaccacggactgagaatcaataatcatagaccttccccttatgtttctgtttgcgattagattctttaaacgctttcttcttatccttatgaacagaagctttattaaaattatgctttgcgactaaattgttcatagtgctgaattacctctcttaaacatttgcatgtgaatgaaaactttttagctacaccacattcaaatatatgttctcttaaatcgcgtgtatcggtatatcccatctcaacaataaaatgccgtattagatttttatctttatcgtttagagaattaaaataatcagattttgaattaatttccctggccaaattgaatcaccttcagttgacgttttaactcttttatcatctcttcgttcatcgcaatataaagatcgcgtagagcaggttttagcattccatttactggagaactaaatggacatacataatcttttcctacgagctttttagtgaattccatatcacagaactgaaatcccggctcattggtataaattccccaattagttgacatcattttattggcatattccagtgcctggatttgattcataattccatcaatttgaaactttttaatattcattagtaaaggtcctcagagtaaagttctttttcactaccacgttcaatacttacttgtccagcgtaagttgcaataatcattgcttcttcacgtgtccaataattactatattggtcaataaacccttggtcatcatcacaaacttgctgagtaactaattgaggtttaactacatctaaaacttctgccatatctttagaataatgacgagcacctggaataataagagttcgtccatcttttaatttaaaacggttggctgcgcaaacaattcgtcgttgatacttttggttttcatcccagtacgcagtctgccaacagatttcaggaacttctttcagaatatcttcttctgtgcatttataaccatgcgctttaaatttttcaacaagactttctggagtttcacgagataaaggaacattcagcatttttaaacgattgataaatgggttcatttaaaccatcctttaatacgctgccacaaagttttctgttgagctttgttgacgccaattgagcgaataaccggttgagattcctggaattctttataatcagcaaggtaaatttcgtaagctgcatccgtaaatgaacttatcgctgccataaaattattgcgaatacctactggagcatctttactttcacgaatgatcatgtatttaccagtcttaatctttacgatagttccaagataagctccatggtaccaaatatcccaaccctcttgagtaggttctgcgcaacgacgaagttcattgacaatttctaacttgtttattatttattcctcacagttcagatgctacagtgattacagcttcaatgttttctgccgagcgtttaatgtcaagatacacattaccgtttttagcgattttacatgacattccgatgtcagtaaatttctgaatatgatgttccatcattttgtatccaaaaattcgcatatttccattgttattaatttcaaaattacgaattccgttagtgcgtttttctaaaatagcaagataattactacgataaatttcaacctttttaagaacaaatccatttttatctaaaagttttaacatgaggtctttatcttcttccatatcggaagtaatctcgcgagctttacgagttgctcgttttttcagcagttccggagcattttcctgtgcatataaagttgctgcatttgaaataatatcctgagcttcaccagtaatgattaatccatcaccagatttctccaccaggccttttttaatcaataccccaatattactattaactactgcgttacctaaatctggatgcacctcacgaacttctgcagctgtaatgaaatctttcttagcaatggtaattaaaatcgtagcagttttttcattcagaacatcgttagaagctttgatgatgtaagttactttagacattttctaatctccgtaattctgtatcagtagttgatagttgtatagtaccacagtatgctttggttgtaaaccgttttgtgaaaaaatttttaaaataaaaaagggagagcctcggctctccctaaaattactgcatgactgtgataactgtcatgataacacgttgaattccgaacgcaagaagacctcctgctacggctggaacaacgcctaaacccgccagtaaaatgctaccagatactaatgcagcgcttgtaataccaatgaatggactcatttgatttcctctaaatctttggtgtattcagtaactacatcagtagttttccaatattcgttttcttcttttttagctttagcttcttcagcaagtttctttgcttcatcggaagtcatatgaaaaatattcattccaactagtttatcaacataagaagaatacatatcgattttcgaaagttcttcggtcagttctttacgagttttaccctgtacaacaatttcacctgaaattactttcttaatgaaatgtgctttggcaaaggctaaacgaaaagctgactcagtttctttaattttgttatcaattcgtttttggacataagttttacgaacttcaacaaagtctttaattaaatcaactacgttatcgtaaacttgcagctttcctttctcattaataaccgtaatattctgggaacgacgctcaatcagtccgaagtctttcataatttttgcatggcgttcttcttcgttatcgctcaaagaatattctttgcggaatttaactttgaagccaaaaccatgctcaccacaagcatcatcccatgtaatgaagcctttattttcaagtgggtctaagattttactcacataagtttcacgatcatacttatacggaatctcagtgatatgcatttgagttcgtgaagtaaacttatatgttccacgaatttcatattgcccatcaatttcaacgacttcaccacgaaattctgggaattctactttcggtttagttactttctttccttgaagagcttgcagtacagctttcttgacagaagaaacactatgaggaagaatgtaagttgcataaccagttgcaataccggaaacgccattaagaagaacagtaggaataataggcaaatagaaagcaggcggaatgtgttctttatcttgatgtaccggagcatattcagtatctttatatacgttatagaaatttttacttacacgagcaaaaatataacgacttgccgctgccttttggacagtacgagaaccaaagtttccttgaccatctaacagaggaaagttattattccaagtattagccatcaaagcacctgcgtcttgtgcagagttttcaccatgatgatatccaaggtccgctacaccacctgcaatagaagcgagtttgtgaaacttatctttatttcctcgtgccaaatcaagagctcgagcaataacaaatcgttgaactggcttaaatccatcaatcatatttgggatagcacgattttcaaccgtgtacatagcataagccaatgcttcattatcaatgatactttttaaatcgcgattattcagttgcataaatttaccatactagtgaatgtagtgccataataacatcagaaatgaaaagcacgacttgaattaatccgaacattactccgtaatatagtgctaccaataaagcagcaagggctaatgaatagcccaagattttcttaatcattagataacaacacaaatgttaaatatgcacacataccctgggctaaagcttgtgaaaacacactgctagcatcgatacagatagttaaaacacatgctactatccaacaaataaatgaaataactcctaataattttgcaatattcatattttcctcactggcgtccgaagacgcctttagttttaagattgttacgatagaactgcatcacgtgttcgttatggaaattactcattaatatgcctgtaaaacaaatttaaagttatcagccaacatacggttcatttcttcgagtgtttgatactcagaatgatgattacgagtaaacgccaaagctagctgaccttttccaaatcccgtcgttagaggtttcatcttagaagcaggcagataaaacactgtgtatggaacattgttatttgcaatagtacgcgcaagttgagaccggcgttgacgaatatgacttaaaaccgcactgaatccttgcttagaacgctgattacctacataaaatcgtgcagatacgcatggattactaaatggaccaccgagtttactcactaaaaagtaaaatccaggtttagataaaatatctttatgcggagttcctaaaaaccattcaccacccttgattgtaccaataacagtagcgcctgcatcattcagatcagtaacagtcatatatttcatattaatttcctctaaattattttctactccaaggccgcatgaatacacacggccattaaattactcgtcgcagtcgacgctcaattcccaaaactcttctacagtataagtttcagtatcattttcaatacagaaacgttcattactattatttgctaaagtagcattaactgtcattttttcgctagtgctcttaagaggtgaaatacgaattaactgatcaccgttatctaaacaaaaaatttcaccaacttttacatctttaaaacttttcataattcacctcaaggagtataaaatccaaatgcagttgttgaccatcccatccaatatggaaaatttacaccaatgtaaaacataagaatataaaaccaaccgctcagcaaattcatcattttacaccattccaaattgtttcaaccacggattttaaaccattttgatgaatatccattcctactaccgccatcaaataaattccaactacaactgaacctaaggcaaaaatcagcatgaaaatgaataaagccggaaaaatattatcgaaaaaccattcaataaatgtaaaagcactgcgtttacgttcatattttcctcacataaatccaaagtaaacgtttaatacatcaatcattaaaacgattgggaatatactcaaaactattagtattataactacattccatatagctttaataatctttttcattttctgttcctccatagttgatagggtaatagtaccacggaagaacagtcttgtaaacaacttttttaaaaatattcgtaataaatgtgaataccaactactaccgctgaaacctgtgcaacccaccacgcacaagcaataagtacagaattcaaaattttcataataacctcattacaaaagtaaatgttaaacaaattactggaatactaattaaccaaacaaaacaccaccataatgaactcatagttcaatctcagcgattttcattttattctccaaatccgtatcagtagttgatagttgtatagtaccacggtccttgtggtatgtaaactgttttgtgaaattttttaaatggaaagataccatccgttgtagttgctttttcttacaactttacgaaggtcttctctgtcaccgatgaacttcggagtgtactggatgacacctggatgaatttctttagtgttgaatataattatacagtcagcgacttgatgatttagaatgggccctagatttattccagaaccatatggatactctccgctgcatcccgttgttaccgaaatccaacgtgagtcagtttgatgtgtcttaacttctacacgaagcccgcagtattttggatgagccaatacatcccatgcatatgtgtacggatcatcgacatcctcttggcctttattgacatatccactcaaccaatctgccacaaaaaactctgcgtacacagcgatacggcatctttcgataacttctgccttatcttgatttgggttttgttttaaagagtatcttgcagtatcagcaattttgaccttcatttcacaggtcaagtcactgttcgatagggtaaatgtcggaatctgaaatagtctctgtaacccaggattcgttttctgcatttaaactttcctttatgtcggaatcaccgatattcatataaatcataatttctcttaaaacaaaaggccgaagccctttattttacttgaattgtgcaattcttttctctagacattcagcataagatttcattgagatgaactgcgaaagtagcagttcttgctcaactgcgctaactgttagaaactttgcgctttctaaaaatttgctcagtgcattaattttgagcattaattgatcgtattcttcttttactcgtgcttgataagctaacataattttccttagttaagggccgaagccttatttaaattgttcagtaacgtcttcaactacttcatattggcaggtacgcattttagcatcgttgtaatcaatcggaattgatactacatcgcgaggatgaactttaacttttacaactcggctggttgaactaccaaagtgacgaatataagatttagaacacacatgcaaaccacgagaacaagtttgtgtatcatcgtcattcacacgagtacgtggcattttaactactttacccggactgttatcaaaggtgtttgagtgacagtcaaagtaattgctgcgaactactttccaagcatagaagtaaccatcttctgtaatttcaatatcgtttgctaccaagaaatcaaagagtcgagataccgctttttggcttgggttttccaacagattttccaagaacggaaaataaaattcaaagttttcgcctttttccatcgagtcaagaatacgatcaaccaaaccagaccgcaattcaatattttgatagaacaagcttccaccttcaattcgaacatcgccggaaatatatttttcaacagcacgacgaacattaattttttgtgccgcttcttccaacttatccgctacaagcagattaagaatttcctggaagtttgaatgagtattaggagttgcgttataagttacgccatcaacagtaattgaaatgaattttttagatgcattccaaataatgtcagatttagcaactggagcaataactgcatcgctattaactttaactgtaatatcaccgctaatagtaactttagggcgtttagcttcttcagcatttttcaaaacacgacggattgtgtcaaccgatacaccttgccaatcagccaattcctgttgggtgtaattaccacttgaatacagtttaacaatttcagcttgttcgtttttggtcaggcatttaatattgtacat

SEQ ID NO: 130 - DNA, 32 bp, Artificial Sequence
  Spacer
tgattgacggctacggtaaaccggcaacgttc

SEQ ID NO: 131 - DNA, 32 bp, Artificial Sequence
  Spacer
gctgttaacgtacgtaccgcgccgcatccggc

SEQ ID NO: 132 - DNA, 32 bp, Artificial Sequence
  Spacer
cggacttagtgccaaaacatggcatcgaaatt

SEQ ID NO: 133 - DNA, 37 bp, Artificial Sequence
  Spacer
gccataatctggatcaggaagtcttccttatccatat

SEQ ID NO: 134 - DNA, 37 bp, Artificial Sequence
  Spacer
ggctttacgccagcgacgtattgccacaggaataact

SEQ ID NO: 135 - protein, 37 aa, Artificial Sequence
  Spacer
GGGGATAGCGCGCCTGGAGCGTGCGATAGAGACTTTG

SEQ ID NO: 136 - DNA, 37 bp, Artificial Sequence
  Spacer
ggcatttaccgaccagcccatcagcagtacagcaaac

SEQ ID NO: 137 - DNA, 37 bp, Artificial Sequence
  Spacer
tcctgaatcaaatccgcctgtggcaggccatagcccg

SEQ ID NO: 138 - DNA, 29 bp, Artificial Sequence
  Repeat Sequence
gagttccccgcgccagcggggataaaccg

SEQ ID NO: 139 - DNA, 29 bp, Artificial Sequence
  Repeat Sequence
gttttatattaactaagtggtatgtaaat

SEQ ID NO: 140 - protein, 101 aa, Unknown
  Viruses
MKTYQEFIVEAKRREDELPFVSKTFDGTLADFKKIPNDKLAKLVGSISETPMNYDRFYPCDFVNDKLALIFEENNDLLLSYVDLITKNYNNPKIKGKQITN

(19) United States
(12) Patent Application Publication
(10) Pub. No.: US 2025/0268963 A1
(43) Pub. Date: Aug. 28, 2025

(54) SYNTHETIC VIRUSES

(71) Applicant: SNIPR Biome ApS, Copenhagen Ø (DK)

(72) Inventors: Jakob Krause HAABER, Copenhagen Ø (DK); Szabolcs SEMSEY, Copenhagen Ø (DK)

(73) Assignee: SNIPR Biome ApS, Copenhagen Ø (DK)

(21) Appl. No.: 18/027,847

(22) PCT Filed: Sep. 24, 2021
(86) PCT No.: PCT/EP2021/076360

§ 371 Date: Mar. 22, 2023

(30) Foreign Application Priority Data
Sep. 26, 2020 (GB) 2015255.9

(51) Int. Cl. A61K 35/76; C12N 7/00

(52) U.S. Cl. CPC A61K 35/76; C12N 7/00; C12N 2795/10021; C12N 2795/10032; C12N 2795/10052; C12N 2795/10062

(57) ABSTRACT

The invention relates to modified viruses that are synthetic, compositions comprising such viruses, virus infectivity assays and methods of selecting modified synthetic viruses. The invention also relates to methods of modifying viruses to produce modified synthetic viruses comprising heterologous nucleic acid (DNA or RNA).

### CROSS REFERENCE TO RELATED APPLICATIONS

This application is a national stage application under 35 U.S.C. § 371 of International Application No. PCT/EP2021/076360, filed internationally on Sep. 24, 2021, which claims priority to and the benefit of European Application No. 2015255.9, filed Sep. 26, 2020, the contents of which are incorporated herein by reference in their entirety.

### REFERENCE TO AN ELECTRONIC SEQUENCE LISTING

The content of the following submission on ASCII text file is incorporated herein by reference in its entirety: a computer readable form (CRF) of the Sequence Listing (file name: 786212001300SEQLIST.TXT, date recorded: Mar. 8, 2023, size: 364,690 bytes.

### TECHNICAL FIELD

The invention relates to methods of modifying viruses to produce modified synthetic viruses comprising heterologous nucleic acid (DNA or RNA). The invention also relates to modified viruses that are synthetic, compositions comprising such viruses, virus infectivity assays and methods of selecting modified synthetic viruses.

### BACKGROUND

The state of the art describes synthetic viruses as vectors for delivering heterologous payloads (eg, DNA or RNA) into target host cells. Examples are engineered lentiviruses, adeno-associated viruses (AAV) and bacteriophage (AKA phage).

The packaging capacity of lentiviruses, adeno-associated viruses (AAV), phage and other viral vectors is finite and usually relatively small, ie, there is usually only a relatively small capacity to package heterologous nucleic acid in the capsids of such viruses, mainly since the genes encoding essential functions (such as virus production and replication) are retained and the resulting virus must be able to infect its target cell so that the virus can introduce the heterologous nucleic acid into the cell.

Where the virus is a lytic virus or temperate virus, such as a temperate phage (ie, which has lytic and lysogenic pathways in its life cycle), lysis functions may be advantageous where the virus is to be used for host cell killing (such as where the heterologous nucleic acid encodes an agent that is toxic to the host), and thus disruption of genes encoding lytic functions is also to be avoided when inserting heterologous nucleic acid.

### SUMMARY OF THE INVENTION

The invention provides the following configurations.

### In a First Configuration

A method of producing a modified genome of a first virus, wherein the modified genome comprises a total number (X) of base pairs of heterologous DNA, wherein the first virus is capable of infecting a target cell of a first species or strain, the method comprising
(a) obtaining sequence(s) of the genome of the first virus at least to the extent comprising a first set of genes required for virus particle production in a host cell; and
(b) producing a hybrid DNA comprising the sequence(s) obtained in step (a) and said heterologous DNA, wherein the hybrid DNA comprises said modified genome;

### Wherein

(c) the modified genome is functional to produce a second virus that is capable of infecting the target cell, the second virus comprising proteins encoded by said set of genes, wherein the proteins package hybrid DNA comprising said heterologous DNA and said set of genes, wherein the second virus is a modified version of the first virus; and
(d) A: the hybrid DNA excludes a total number (Y) of base pairs of DNA of the genome of the first virus wherein Y is at least 49% of X; or
   B: the second virus comprises a capsid that has a DNA packaging capacity of Zbp and the total number of base pairs of the hybrid DNA is 90-110% of Z.

In an alternative (such as wherein each virus is an RNA virus), instead of heterologous DNA, the modified genome comprises RNA. Thus in the alternative herein, where DNA is mentioned (such as part of the virus). RNA may be used instead and the disclosure is to be read mutatis mutandis as relating to RNA instead of DNA.

### In a Second Configuration

In a first aspect: A method of producing synthetic virus particles, comprising carrying out the method of the First Configuration to produce the hybrid DNA, introducing the hybrid DNA into a target cell of a first species or strain in which the hybrid DNA is capable of being replicated and particles of said second virus are produced; and producing second viruses in the cell; and further optionally isolating second virus particles from the cell.

In another aspect: A method of producing synthetic virus particles, introducing hybrid DNA obtainable by the method of the first configuration into a target cell of a first species or strain in which the hybrid DNA is capable of being replicated and particles of said second virus are produced, and producing second viruses in the cell; and further optionally isolating second virus particles from the cell.

### In a Third Configuration

A method of selecting a synthetic virus, the method comprising
(a) Carrying out the method of the Second Configuration to produce a first type (T1) of said second virus;
(b) Carrying out the method of the Second Configuration to produce a second type (T2) of said second virus, wherein T1 and T2 differ from each other by at least said heterologous DNA comprised by each type (eg, T1 and T2 differ by heterologous DNA encoding first and second tail fibres respectively, wherein the tail fibres are different);
(c) Culturing the T1 virus with target cells of the first species or strain; and culturing the T2 virus with target cells of the first species or strain;
(d) Determining which of the cultured T1 and T2 viruses produces a predetermined indicator or the extent of production of the indicator by the viruses;
(e) Selecting T1 or T2 virus on the basis of the determination in step (d); and
(f) Optionally further producing further copies of the selected virus and/or determining the sequence of the heterologous DNA or a portion thereof comprised by the selected virus.

### In a Fourth Configuration

A virus infectivity assay, the assay comprising
(a) providing a first type (T1) of virus comprising a first DNA sequence,
(b) providing a second type (T2) of virus comprising a second DNA sequence, wherein T1 and T2 differ from each other by said DNA sequences;
(c) Culturing the T1 virus with target cells of a first species or strain; and culturing the T2 virus with target cells of the first species or strain;
(d) Determining which of the cultured T1 and T2 viruses produces a predetermined indicator or the extent of production of the indicator by the viruses;
(e) Selecting T1 or T2 virus on the basis of the determination in step (d); and
(f) Optionally further producing further copies of the selected virus and/or determining the sequence of said DNA or a portion thereof comprised by the selected virus.

### In a Fifth Configuration

In a first aspect:

A synthetic phage, wherein the phage is
(i) a synthetic T-even phage (eg, a T4, T2 or T6 phage, preferably a T4 phage) comprising a deletion of DNA from a Deletion Permissive Region (DPR) of the genome of the phage, wherein the region is between the pin (protease inhibitor) gene and the iPII (internal protein) gene; or
(ii) a synthetic version of a phage that is not a T-even phage, wherein the synthetic phage comprises a deletion of DNA from a region of its genome that is homologous or orthologous to said DPR; and
wherein the synthetic phage is capable of replication in a host cell.

In a further aspect:

A synthetic phage, wherein the phage is
(i) a synthetic T4 phage comprising a deletion of DNA from a Deletion Permissive Region (DPR) of the genome of the phage, wherein the region is between the pin (protease inhibitor) gene and the iPII (internal protein) gene; or
(ii) a synthetic version of a phage that is not a T4 phage, wherein the synthetic phage comprises a deletion of DNA from a region of its genome that is homologous or orthologous to said DPR; and wherein the synthetic phage is capable of replication in a host cell.

### In a Sixth Configuration

In a first aspect:

A synthetic phage, wherein the phage is
(i) a synthetic T-even phage comprising an insertion of heterologous DNA into a Deletion Permissive Region (DPR) of the genome of the phage, wherein the region is between the pin (protease inhibitor) gene and the ipII (internal protein) gene; or
(ii) a synthetic version of a phage that is not a T-even phage, wherein the synthetic phage comprises an insertion of heterologous DNA into a region of its genome that is homologous or orthologous to said DPR; and
wherein the synthetic phage is capable of replication in a host cell.

In a further aspect:

A synthetic phage, wherein the phage is
(i) a synthetic T4 phage comprising an insertion of heterologous DNA into a Deletion Permissive Region (DPR) of the genome of the phage, wherein the region is between the pin (protease inhibitor) gene and the ipII (internal protein) gene; or
(ii) a synthetic version of a phage that is not a T4 phage, wherein the synthetic phage comprises an insertion of heterologous DNA into a region of its genome that is homologous or orthologous to said DPR; and
wherein the synthetic phage is capable of replication in a host cell.

### In a Seventh Configuration

In a first aspect:

A synthetic phage, wherein the phage is
(a) a synthetic rV5 or rV5-like phage comprising a deletion of DNA from a Deletion Permissive Region (DPR) of the genome of the phage, wherein the region is between gene 39 and gene 46 or between gene 230 and gene 240; or
(b) a synthetic version of a phage that is not a rV5 or rV5-like phage, wherein the synthetic phage comprises a deletion of DNA from a region of its genome that is homologous or orthologous to said DPR; and
wherein the synthetic phage is capable of replication in a host cell.

In a further aspect:

A synthetic phage, wherein the phage is
(a) a synthetic Phi92 phage comprising a deletion of DNA from a Deletion Permissive Region (DPR) of the genome of the phage, wherein the region is between gene 39 and gene 46 or between gene 230 and gene 240; or
(b) a synthetic version of a phage that is not a Phi92 phage, wherein the synthetic phage comprises a deletion of DNA from a region of its genome that is homologous or orthologous to said DPR; and
wherein the synthetic phage is capable of replication in a host cell.

### In an Eighth Configuration

In a first aspect:

A synthetic phage, wherein the phage is
(a) a synthetic rV5 or rV5-like phage comprising an insertion of DNA into a Deletion Permissive Region (DPR) of the genome of the phage, wherein the region is between gene 39 and gene 46 or between gene 230 and gene 240; or
(b) a synthetic version of a phage that is not a rV5 or rV5-like phage, wherein the synthetic phage comprises an insertion of DNA into a region of its genome that is homologous or orthologous to said DPR; and
wherein the synthetic phage is capable of replication in a host cell.

In a further aspect:

A synthetic phage, wherein the phage is
(a) a synthetic Phi92 phage comprising an insertion of DNA into a Deletion Permissive Region (DPR) of the genome of the phage, wherein the region is between gene 39 and gene 46 or between gene 230 and gene 240; or
(b) a synthetic version of a phage that is not a Phi92 phage, wherein the synthetic phage comprises an insertion of DNA into a region of its genome that is homologous or orthologous to said DPR; and
   wherein the synthetic phage is capable of replication in a host cell.

### In a Ninth Configuration

A synthetic phage, wherein the phage is
(a) a synthetic T4 phage that comprises a deletion of DNA from, and/or an insertion of heterologous DNA into, a region of the genome of the phage corresponding to a region between coordinates
(i) 1887 and 8983;
(ii) 2625 and 8092;
(iii) 1904 and 8113;
(iv) 2668 and 7178;
(v) 7844 and 11117;
(vi) 8643 and 10313;
(vii) 8873 and 12826;
(viii) 9480 and 12224;
(ix) 8454 and 17479; or
(x) 9067 and 16673;
wherein coordinates are with reference to wild-type T4 phage genome (SEQ ID NO: 129); or
(b) a synthetic version of a phage (eg, a T-even phage) that is not a T4 phage, wherein the synthetic phage comprises a deletion of DNA from a region of its genome that is homologous or orthologous to said region of (a);
and
wherein the synthetic phage is capable of replication in a host bacterial cell.

A method of producing a synthetic phage, the method comprising
(a) providing a heterologous DNA comprising an insert;
(b) providing a first phage genomic DNA;
(c) allowing homologous recombination between a first region of the genomic DNA and the heterologous DNA and allowing homologous recombination between a second region of the genomic DNA and the heterologous DNA,
wherein the insert is inserted between said regions whereby a hybrid DNA is produced that encodes the genome of a synthetic phage; and
wherein
A:
(i) the coordinates of the first region are 1887-2625 and the coordinates of the second region are 8092-8983;
(ii) the coordinates of the first region are 1904-2668 and the coordinates of the second region are 7178-8113;
(iii) the coordinates of the first region are 7844-8643 and the coordinates of the second region are 10313-11117;
(iv) the coordinates of the first region are 8873-9480 and the coordinates of the second region are 12224-12826; or
(v) the coordinates of the first region are 8454-9067 and the coordinates of the second region are 16673-17479;
wherein the first phage is a T4 phage and the coordinates are with reference to wild-type T4 phage genome (SEQ ID NO: 129);
or
B: the first phage (eg, a T-even phage) is not a T4 phage, and wherein the first and second regions are regions of the first phage genome that are homologous or orthologous to said first and second regions of any one of A(i) to (v).

A synthetic phage obtainable by the method of the ninth configuration; or a composition comprising a plurality of synthetic phages, wherein each phage is obtainable by the method of the ninth configuration.

Aspects also provide pharmaceutical compositions, methods of making such compositions and medical methods using such compositions. The invention also provides a virus (eg, a phage) obtained or obtainable by any method disclosed herein, as well as a plurality of said viruses.

### BRIEF DESCRIPTION OF THE DRAWINGS

FIG. 1. Transfer of CRISPR components from the recombination donor plasmid to the phage chromosome. Recombination of the UHS and DHS with their homologous sequences on the phage chromosome resulted in a CRISPR armed phage in which a piece of the phage chromosome was replaced by the CRISPR system.

FIG. 2. Outline of the deletion-scanning strategy to find the optimal location of the CRISPR system in SA117. The relevant part of the phage chromosome is shown on the top, with the homologues of the pin and lysis genes flanking the DPR deletion in T4. Numbering shows the location of genes on the SA117 phage sequence Arrowheads indicate the direction of genes of known functions. In the arming process about 7800 base pairs of the SA117 chromosome is replaced with a CRISPR system with an equivalent length shown below the sequence. The CRISPR system consists of the E. coli cas3 to casE genes and a corresponding array containing 3-5 spacer sequences targeting conserved genes. Transcription of the CRISPR system is driven from promoter P.

### DETAILED DESCRIPTION

The invention relates to methods of modifying viruses to produce modified synthetic viruses comprising heterologous nucleic acid (DNA or RNA). The invention also relates to modified viruses that are synthetic, compositions comprising such viruses, virus infectivity assays and methods of selecting modified synthetic viruses.

The invention is based on considerations of the finite and relatively restricted nucleic acid packaging capacities of viral vectors. Without reduction of the virus genome as per the invention, desired virus production may be prevented or very inefficient for packaging heterologous DNA (eg, not packaging all of the desired DNA or yielding a low phage titre) The invention addresses this problem for heterologous DNAs, where the total number of base pairs of the hybrid DNA is near or exceeds the packaging capacity (ie, 90-110% of the packaging capacity). The invention frees up space (eg, by removing virus genomic DNA to at least 50% of the size of the heterologous DNA) whilst preserving genes required for virus replication and production, to produce a virus that packages the heterologous DNA and is capable of infecting a target cell. The invention is especially useful when engineering lytic viruses (eg, lytic phages), as the genomes of these does not comprise dispensable elements such as lysogenic pathway genes found in temperate viruses (eg, temperate phage).

The virus, method, assay or composition may be useful to provide one or more of the following advantages:—
(a) producing viable phage that have reduced native phage genomes but with the addition of heterologous nucleic acid (such are viable in that they retain at least the host range specificity of the unmodified, starting phage);
(b) identification of Deletion Permisive Regions in phage, such as T-even phage, that permit modification;
(c) Modified phage assays that enable selection of viable phage that comprise hybrid genomes, wherein the hybrid genomes comprise heterologous nucleic acid (such as one or more sequences encoding a phage tail fibre or component thereof—useful for selecting phage with alterered (eg, extended) host range specificity).

In a first configuration, there is provided:—

A method of producing a modified genome of a first virus, wherein the modified genome comprises a total number (X) of base pairs of heterologous nucleic acid, wherein the first virus is capable of infecting a target cell of a first species or strain, the method comprising
(a) obtaining sequences) of the genome of the first virus at least to the extent comprising a first set of genes required for virus particle production in a host cell; and
(b) producing a hybrid nucleic acid comprising the sequence(s) obtained in step (a) and said heterologous nucleic acid, wherein the hybrid nucleic acid comprises said modified genome;

### Wherein

(c) the modified genome is functional to produce a second virus that is capable of infecting the target cell, the second virus comprising proteins encoded by said set of genes, wherein the proteins package hybrid nucleic acid comprising said heterologous nucleic acid and said set of genes, wherein the second virus is a modified version of the first virus; and
(d) A: the hybrid nucleic acid excludes a total number (Y) of base pairs of nucleic acid of the genome of the first virus wherein Y is at least 50% of X; or
   B: the second virus comprises a capsid that has a nucleic acid packaging capacity of Zbp and the total number of base pairs of the hybrid nucleic acid is 90-110% of Z.

In an alternative, Y is at least 49% of X, as per the Example herein. In an alternative, Y is at least 55% of X, as per the Example herein.

In one aspect, the first virus is a T-even phage and Z is from 165000 to 180000 bp, eg, the first virus is a T4 phage and Z is from 168000 to 177000 bp (such as 168903 bp±5%). In one aspect, the first virus is an rV5-like phage, eg, a Phi92 phage, and Z is from 140000-150000 bp (such as 148612 bp±5%).

In one aspect, Z is no less than 80000 bp, eg, wherein the first virus is a Felix O1 phage. In one aspect, Z is no more than 500000, 400000, 300000, 200000 or 100000 bp. For example, the first virus is a Jumbo Phage (see, eg, Front Microbiol 2017 Mar. 14; 8:403, doi: 10.3389/fmicb.2017.00403. eCollection 2017, “Jumbo Bacteriophages: An Overview”, Yihui Yuan & Meiying Gao, PMID: 28352259, PMCID: PMC5348500, DOI: 10.3389/fmicb.2017.00403). In one aspect, Z, is more than 200000 bp, and optionally no more than 500000, 400000 or 300000 bp.

In one aspect, such as wherein the heterologous DNA comprises or encodes one or more components of a CRISPR/Cas system, X is 5000-7000 bp. For example, such heterologous DNA encodes one or more (eg, 1, 2, 3, 4, 5, 6, 7, 8, 9 or 10) different crRNAs or guide RNAs; and/or encodes one or more (eg, one or two) Cas. The DNA may encode a Cas9 and/or a Cas3. The DNA may encode a Type V Cas. The DNA may encode a Cas12 or Cas13,

In one aspect, each virus is a DNA virus and the nucleic acid is DNA.

In an example, the first set of genes are required for virus particle production in a host cell and host cell infection. For example, the first set of genes comprises genes that encode viral structural proteins and genes that are required for DNA replication.

In step (c) a standard phage infectivity assay may be used to determine that the modified genome is functional to produce a second virus that is capable of infecting the target cell, such as a plaque assay, for example wherein phage infection of a lawn of target bacterial cells is determined by detecting plaques in the lawn. In an embodiment, the second virus is determined as being capable of infecting the target cell in a plaque assay that determines the presence of at least 10 pfu/ml when a lawn prepared by plating 1e7 to 1e8 target cells on an agar plate is contacted with at least 1 of the second virus per 100 microlitres for 12-18 hours. Preferably, the assay determines the presence of at least 1e7, 1e8, 1e9, 1e10, 1e11, 1e12, 1e13 or 1e14 pfu/ml.

For example, the virus of (a) is capable of lysing the target host cell and the set of genes comprises (iii) genes that are required for target cell lysis and/or (iv) genes that are required for target cell DNA degradation. Optionally, the virus is a bacteriophage and the cell is a bacterial cell; or the cell is an archacal cell and the virus of (a) is a virus that is capable of infecting the archaeal cell.

In an alternative (such as wherein each virus is an RNA virus), instead of heterologous DNA, the modified genome comprises RNA. Thus, in the alternative herein, where DNA is mentioned (such as part of the virus), RNA may be used instead and the disclosure is to be read mutatis mutandis as relating to RNA instead of DNA Thus, in one aspect, each virus is a RNA virus (eg. a retrovirus) and the nucleic acid is RNA.

When the first virus is a T4 phage, for example, the first set of genes may comprise the genes of Table 5; or when the first virus is a T-even phage that is not a T4 phage, the first set of genes may comprise homologues or orthologues of the genes of Table 5.

There is provided:—

A method of producing a modified genome of a first virus (eg, a DNA virus, such as a phage), wherein the modified genome comprises a total number (X) of base pairs of heterologous DNA, wherein the first virus is capable of infecting a target cell of a first species or strain, the method comprising
(a) obtaining one or more sequences of the genome of the first virus at least to the extent comprising a first set of genes required for virus particle production in a host cell; and
(b) producing a hybrid DNA comprising the sequence(s) obtained in step (a) and said heterologous DNA, wherein the hybrid DNA comprises said modified genome;

### Wherein

(c) the modified genome is functional to produce a second virus that is capable of infecting the target cell, the second virus comprising proteins encoded by said set of genes, wherein the proteins package hybrid DNA comprising said heterologous DNA and said set of genes, wherein the second virus is a modified version of the first virus; and
(d) A: the hybrid DNA excludes a total number (Y) of base pairs of DNA of the genome of the first virus wherein Y is at least 50% of X; or
   B: the second virus comprises a capsid that has a DNA packaging capacity of Zbp and the total number of base pairs of the hybrid DNA is 90-110% of Z.

There is provided:—

A method of producing a modified genome of a first virus (eg, an RNA virus such as a retrovirus), wherein the modified genome comprises a total number (X) of base pairs of heterologous RNA, wherein the first virus is capable of infecting a target cell of a first species or strain, the method comprising
(a) obtaining sequence(s) of the genome of the first virus at least to the extent comprising a first set of genes required for virus particle production in a host cell; and
(b) producing a hybrid RNA comprising the sequence(s) obtained in step (a) and said heterologous RNA, wherein the hybrid RNA comprises said modified genome;

### Wherein

(c) the modified genome is functional to produce a second virus that is capable of infecting the target cell, the second virus comprising proteins encoded by said set of genes, wherein the proteins package hybrid RNA comprising said heterologous RNA and said set of genes, wherein the second virus is a modified version of the first virus; and
(d) A: the hybrid RNA excludes a total number (Y) of base pairs of RNA of the genome of the first virus wherein Y is at least 50% of X; or
   B: the second virus comprises a capsid that has a RNA packaging capacity of Zbp and the total number of base pairs of the hybrid RNA is 90-110% of Z.

By “heterologous nucleic acid” or “heterologous DNA” it is meant that the nucleic acid (or DNA) is not comprised by the unmodified genome of the first virus. In an example, the first virus is a naturally-occurring or wild-type virus, such as naturally found in an environment or in or on an organism (such as a bacterium, prokaryote, eukaryote, mammal, human, human cell, animal, animal cell or plant (eg, a tobacco or tomato plant). In another example, the first virus is a synthetic virus, eg, whose genome has been produced using recombinant DNA technology. In an example, the first virus is not a naturally-occurring or not a wild-type virus.

In an alternative, the invention relates to a non-self-replicative transduction particle instead of a “synthetic phage” or “synthetic virus”. A “non-self-replicative transduction particle” refers to a particle, (eg, a phage or phage-like particle; or a particle produced from a genomic island (eg, a S aureus pathogenicity island (SaPI)) or a modified version thereof) capable of delivering a nucleic acid molecule of the particle (eg, encoding an antibacterial agent or component) into a host cell, but does not package its own replicated genome into the transduction particle. In this alternative, said first set of genes are genes essential for producing the particle and for transduction of a host cell.

Packaging capacities are known in the art for some phage. One may use variations of gel-electrophoresis, such as using Pulsed-field Gel Electroplioresis (PFGE). For example, see methodology disclosed in the textbook Bacteriophages. Methods and Protocols, Volume 2: Molecular and Applied Aspects (Eds. Martha Clokie and Andrew Kropinski), Chapter 3: Determination of Bacteriophage Genome Size by Pulsed-Field Gel Electrophoresis by Erika Lingohr, Shelley Frost and Roger P. Johnson.

The target cell may be a prokarvote cell (eg, a bacterial or archaeal cell), a eukaryotic cell, a mammalian cell (eg, a human, non-human animal, fungal, protozoan, yeast or plant (eg, a tobacco or tomato plant) cell).

The target cell may be a bacterial cell of a genus or species selected from Table 1.

Y may be 90-200% (eg, 90-150 or 90-110 or 90-100%) of X, Y may be at least 50, 60, 70, 80, 85, 90, 95, 96, 97, 98 or 99% of X, Y may be up to 300, 250, 200, 150, 140, 130, 120, 110 or 100% of X, Y may be at least 50, 60, 70, 80, 85, 90, 95, 96, 97, 98 or 99%; and Y may be up to 300, 250, 200, 150, 140, 130, 120, 110 or 100% of X, Y may be from 49 to 106% of X or from 55 to 106% of X, as shown in the examples.

The total number of base pairs of the hybrid DNA may be 90-100% of Z. The total number of base pairs of the hybrid DNA may be 100% of Z.

The parameter Z (packaging capacity) may be the size of the first phage genome. In an example, the size of the hybrid DNA is from 90-110% (eg, from 99-105%) of the size of the genome of the first phage, for example about 100% of the first phage genome.

In an embodiment, the net amount of base pairs of nucleic acid (eg, DNA) that are added to the genome is from −500 to 4000 bp, eg, from 400 to 4000 or 3000 bp, from 200 to 4000 or 3000 bp, or from 100 to 4000 or 3000 bp.

The life cycle of the first and/or second virus may comprise a lytic pathway. The first and/or second virus may be a lytic virus. The first virus may be a temperate virus and the second virus may be a modified temperate virus (eg, wherein the life cycle of the modified virus does not comprise a lysogenic pathway or wherein the lysogenic pathway has been disrupted). Disruption here may be to favour the lytic pathway over the lysogenic pathway and/or to reduce the chances of the second virus entering the lysogenic pathway compared to first virus.

The method may comprise
(i) obtaining DNA from a said first virus, wherein the DNA comprises said set of genes;
(ii) sequencing the DNA of step (i);
(iii) comparing the sequence of the DNA obtained in step (ii) with a reference viral genome sequence, by
   I. aligning the DNA sequence obtained in step (ii) with the reference sequence;
   II. identifying a reference set of genes comprised by the reference sequence wherein the genes are genes required for reference virus particle production and replication;
   III. identifying in the aligned DNA sequence said first set of genes wherein the first set of genes corresponds to the reference set of genes; and
(iv) producing said hybrid DNA comprising said first set of genes identified in step III and said heterologous DNA.

Steps I and II can be carried out in any order.

The skilled addressee will be familiar with methods for aligning DNA sequences to perform step I. For example, one may use nucleotide BLAST (blastn) with default parameters to carry out the alignment (eg, see the blastn suite tool provided by NCBI, such as at https://blast.ncib.nlm.nih.gov/Blast.cgi?PROGRAM=blastn&PAGE_TYPE=BlastSearch&LINK_LOC=blasthome), which searches the ‘nucleotide collection’ that comprises GenBank, EMBL, DDBJ, PDB and RefSeq sequences). In an example, the alignment in step I is carried out using a reference sequence comprised by a GenBank, EMBL, DDBJ, PDB or RefSeq database.

Preferably, the BLAST (blastn or tblastn (see below for blastn)) is version 2.10.1 released on 8 Jun. 2020.

### Default Parameters for Blastn:

General Parameters:

Max target sequences: 100

Short queries: Automatically adjust parameters for short input sequences

Expect threshold: 10

Word size: 11

Max matches in a query range: 0

Scoring Parameters:

Match/Mismatch Scores: 2, −3

Gap Costs: Existence 5; Extension 2

Filters and Masking:

Filter: Low complexity regions

Mask: Mask for lookup table only

Discontiguous Word Options:

Template length: 18

Template type: Coding

### Default Parameters for Tblastn:

General Parameters:

Max target sequences: 100

Expect threshold: 10

Word size: 6

Max matches in a query range: 0

Scoring Parameters:

Matrix: BLOSUM62

Gap Costs: Existence: 11; Extension: 1

Compositional adjustments: Conditional compositional score matrix adjustment

Filters and Masking:

Filter: Low complexity regions filter

In step III, the nucleotide sequence of each gene of the first set may correspond when at least 80, 85, 90, 91, 92, 93, 94 95, 96, 97, 98 or 99% (eg, at least 90%) identical to the nucleotide sequence of a gene of the reference set.

The first virus and the virus of the reference sequence may be the same virus or viruses of the same phylum, order, rank or class. For example, they are both enterobacteria phage, E coli phage, Myoviridae phage, Tevenvirinae phage, Tequatrovirus phage, Caudovirales phage, adeno-associated viruses (AAV), herpes simplex viruses, retroviruses or lentiviruses. For example, they are both phage from a genus selected from Dhakavirus, Gaprivervirus, (Gelderlandvirus, Jiaodavinrs, Karanvirus, Krischvirus, Afoonvirus, Afosigvirus, Schizotequatrovirus, Slopekvirus and Tequatrovirus.

Each virus or phage herein may be an enterobacteria phage, E coli phage, Alvoviridae phage, Tevenvirinae phage, Tequatrovirus phage, Caudovirales phage, adeno-associated viruses (AAV), herpes simplex viruses, retroviruses or lentiviruses. For example, each virus or phage herein may be from a genus selected from Dhakavirus, Gaprivervirus, Gelderlandvirus, Jiaodavirus, Karamvirus, Krischvirus, Moonvirus, Mosigvirus, Schizotequatrovirus, Slopekvirus and Tequatrovirus.

Each virus or phage herein may be a Klebsiella virus (eg, Klebsiella phage PMBT1, Klebsiella phage PKO111, Klebsiella phage phi KpNIH-6, Klebsiella phage Miro, Klebsiella phage vB_KpnM_KpV477, Klebsiella phage KPV15, Klebsiella phage vB_Kpn_F48, Klebsiella phage KPN5, Klebsiella phage KP27, Klebsiella phage KP15, Klebsiella phage KP1 or Klebsiella phage JD18), Acinetobacter virus (eg, Acinetobacter virus 133), Aeromonas virus (eg, Aeromonas virus 65 or Aeromonas virus Aeh1), Escherichia virus (eg, Escherichia virus RB16, Escherichia virus RB32 or Escherichia virus RB43) or Pseudomonas virus (eg, Pseudomonas virus 42).

Each virus or phage herein may be a Tevenvirinae phage, eg, a phage selected from Table 6.

Recombinant DNA technology and/or DNA synthesis may be used to produce said hybrid DNA, as will be apparent to the skilled addressee.

Step III may comprise
IV. identifying open reading frame (ORF) sequences in the aligned sequence (First Set ORFs) and comparing the First Set ORFs with ORFs in the reference sequence, wherein ORFs of the aligned sequence that correspond to ORFs of the reference sequence that are comprised by said reference set of genes are identified, whereby genes of the first set are identified as genes comprising the First Set ORFs.

Step IV may comprise

Step V. BLAST analysis of the sequence obtained in step (ii) with viral genome sequences comprised by a database comprising viral genome sequences, optionally a Genbank database. In an example, the database is selected from a GenBank, EMBL, DDBJ, PDB and a RefSeq database.

For example, one or more ORFs are identified either by (i) nucleotide BLAST (blastn) comparing the First Set ORF sequences to sequences in a nucleotide sequence collection (eg, a database selected from a GenBank, EMBL, DDBJ, PDB and a RefSeq database), or (ii) using ‘tblastn’ which uses the protein encoded by the First set ORF as a query and searches all potential protein sequences encoded by a nucleotide sequence collection (a translated nucleotide database). Default parameters of blastn or tblastn may be used. See, the blastn suite tool provided by NCBI, such as at https://blast.ncib.nlm.nih.gov/Blast.cgi?PROGRAM=blastn&PAGE_TYPE=BlastSearch&LINK_LOC=blasthome, which searches the ‘nucleotide collection’ that comprises GenBank, EMBL, DDBJ, PDB and RefSeq sequences New phage sequences are typically automatically annotated by using RAST (Aziz. R. K., Bartels, D., Best. A. A. et al. The RAST Server: Rapid Annotations using Subsystems Technology. BMC Genomics 9, 75 (2008). https://doi.org/10.1186/1471-2164-9-75).

Each genome sequence may be a complete genome sequence of a respective virus. Each of a plurality of said genome sequences may be a complete genome sequence of a respective virus. Each genome sequence may be 90% or more of a complete genome sequence of a respective virus. Each of a plurality of said genome sequences may be 90% or more of a complete genome sequence of a respective virus.

Step (iv) may comprise
VI. deleting at least Xbp of DNA from a DNA comprising the first virus genome to produce a second DNA, wherein the deletion does not include nucleotides of the first set of genes or does not render the first set of genes non-functional for virus replication and production; and inserting the heterologous DNA into the second DNA to produce the hybrid DNA;
VII. inserting the heterologous DNA into a DNA comprising the first virus genome to produce a second DNA; and deleting from the second DNA at least Xbp of DNA to produce the hybrid DNA, wherein the deletion does not include nucleotides of the first set of genes or does not render the first set of genes non-functional for virus replication and production; or
VIII. carrying out said deletion and insertion simultaneously on a DNA comprising the first virus genome, thereby producing the hybrid DNA.

The deletion and insertion of VI or VII may be simultaneous or sequential.

The deletion does not include nucleotides of the first set of genes or does not render the first set of genes non-functional for virus infectivity of the target cell.

There is provided an aspect of the method wherein: (i) Xbp is 2-15 kbp and/or Ybp is 1-20 kbp; or (ii) Y is 50-200% (optionally, 50-100%) of X; or (iii) Zbp is 4 to 600 kbp. For example in (i), Xbp is at least 2, 3, 4, 5, 6, 7, 8, 9, 10, 11, 12, 13, 14 or 15 (but no more than 15) kbp and/or Ybp is at least 1, 2, 3, 4, 5, 6, 7, 8, 9, 10, 11, 12, 13, 14, 15, 16, 17, 18, 19 or 20 (but no more than 20) kbp. For example in (ii), Ybp is no more than 200, 150, 140, 130, 120, 110, 100, 90, 80, 70 or 60% of Xbp and/or no less than 50, 60, 70, 80, 90 or 100% of Xbp. For example in (iii), Xbp is at least 2, 3, 4, 5, 6, 7, 8, 9, 10, 11, 12, 13, 14 or 15 (but no more than 15) kbp. For example in (iii), Zbp is 10 to 550 kbp, optionally wherein the first virus is a dsDNA virus. The inventors determined suitable sizes for specific types of viruses, such as T-even and T-odd phages and other viruses as set out below.

Zbp may be 10 to 550 kbp, optionally wherein the first virus is a dsDNA virus.

Zbp may be 150 to 170 kbp, optionally wherein the first virus is a T-even phage (eg, T4).

Zbp may be 40 to 130 kbp, optionally wherein the first virus is a T-odd phage (eg, T1. T3, T5 or 17).

Zbp may be 30 to 200 kbp, optionally wherein the first virus is a phage (eg, T1, T2, T3, T4, T5, T6, T7, P1, P2, lambda or phi92).

Zbp may be 155 to 175 kbp, optionally wherein the first virus is a T4 phage.

Zbp may be 90 to 110 kbp, optionally wherein the first virus is a T1 phage.

Zbp may be 115 to 130 kbp, optionally wherein the first virus is a T5 phage.

Zbp may be 30 to 50 kbp, optionally wherein the first virus is a T3 or T7 phage.

Zbp may be 85 to 100 kbp, optionally wherein the first virus is a P1 phage.

Zbp may be 25 to 40 kbp, optionally wherein the first virus is a P2 phage.

Zbp may be 35 to 55 kbp, optionally wherein the first virus is a lambda phage.

Zbp may be 140 to 160 kbp, optionally wherein the first virus is a phi92 phage.

Zbp may be 4 to 55 kbp, optionally wherein the first virus is a AAV virus.

Zbp may be 5 to 12 kbp, optionally wherein the first virus is a lentivirus virus.

Zbp may be 5 to 15 kbp, optionally wherein the first virus is a retrovirus.

The heterologous may DNA encode a first viral tail fibre or component thereof and/or the excluded DNA encodes a second viral tail fibre or component thereof, wherein the first and second tail fibres or components are different from each other. Preferably, the second viral tail fibre or component is a fibre or component not comprised by the first virus. Thus, this usefully enables production of second viruses that comprise tail fibres that are not comprised by the first virus and thus may be useful for producing a host specificity of the second virus that is different to the specificity of the first virus (eg, the second virus can infect host cells of a strain or species that cannot be infected by the first virus, or the second virus more efficiently infects such host cells than the first virus). A component may be a tail fibre subunit.

The heterologous DNA may encode a protein (eg, a human protein) or RNA (eg a guide RNA). The protein may be an antibody (or fragment thereof, such as a variable domain or single variable domain), hormone, enzyme, receptor, coagulation factor, cell adhesion protein. RNA-binding protein or DNA-binding protein.

The heterologous DNA may encode a guided nuclease (optionally a Cas) and/or a guide RNA and/or the heterologous DNA comprises a CRISPR array for producing a crRNA in the target cell. The guided nuclease may be a Cas nuclease (eg, a Type I, II, III, IV, V or VI Cas nuclease, eg, a Cas9, a Cas3, a Cas12, or a Cas13). The guided nuclease may be a TALEN, zinc finger nuclease or meganuclease.

The heterologous DNA may comprise or consist of from 1 to 10 kb, eg, 1 to 9, 1 to 8, 1 to 7, 1 to 6, 1 to 5, 1 to 4, 1 to 3 or 1 to 2 kb, of DNA. For example, the heterologous DNA comprises a CRISPR array (and/or a nucleotide sequence encoding a guide RNA, such as a single guide RNA) and optionally one or more nucleotide sequences which each encodes a respective Cas. In addition or alternatively, the heterologous DNA may comprise a nucleotide sequence encoding a virus (eg, phage) tail fibre or a component thereof.

DNA sequences encoding Cas proteins can be relatively large, and thus the invention finds benefit when the heterologous DNA encodes one or Cas. Without reduction of the virus genome as per the invention, second virus production may be prevented or very inefficient (eg, not packaging all of the desired DNA or yielding a low phage titre) when it is desired to package heterologous DNA. The invention addresses this problem for heterologous DNAs, where the total number of base pairs of the hybrid DNA is near or exceeds the packaging capacity (ie, 90-110% of the packaging capacity) The invention frees up space (eg, by removing virus genomic DNA to at least 50% of the size of the heterologous DNA) whilst preserving genes required for virus replication and production, as well as infectivity of a target cell, thereby enabling production of desired viruses that package the heterologous DNA.

The heterologous DNA may encode a CRISPR Cascade protein (eg, Cas A, B, C, D or E)

The heterologous DNA may encode a crRNA. The heterologous DNA may encode a single guide RNA (sgRNA). The heterologous DNA may encode a tracrRNA.

The heterologous DNA may encode an antibacterial agent that is toxic to the target cell, wherein the target cell is a bacterial cell. The heterologous DNA may encode an agent that is toxic to the target cell, wherein the target cell is an archaeal, yeast or algal cell. The heterologous DNA may encode an agent that is toxic to an organism comprising the target cell, eg, wherein the organism is an insect, plant, protozoan, fungus, yeast or any other organism disclosed herein (optionally not a human).

The heterologous DNA may encode a protein (eg, a human protein) or RNA (eg a guide RNA) The protein may be an antibody (or fragment thereof, such as a variable domain or single variable domain), hormone, enzyme, receptor, coagulation factor, cell adhesion protein, RNA-binding protein or DNA-binding protein.

The heterologous DNA may encode a virus tail fibre and a guide RNA (eg, a single-guide RNA). The heterologous DNA may encode a virus tail and comprises a CRISPR array for producing a crRNA in the target cell.

Each virus may be a DTR virus (eg, a DIR phage), which comprise Direct Terminal sequence Repeats that mark the beginning and the end of the virus genome. The advantage of these viruses is that they possess a sequence specific DNA packaging mechanism and therefore generally do not transduce host genes. For example, a virus herein may be a phage of the rv5-like group of phage, such as Phi92.

Each virus may be a phage (eg, an enterobacteria phage, E coli phage, or Caudovirales phage (such as a Myoviridae phage, Tevenvirinae phage or Tequatrovirus phage)), adeno-associated virus (AAV), herpes simplex virus, retrovirus or lentivirus. For example, both the first and second viruses are be a phage (eg, an enterobacteria phage, E coli phage, or Caudovirales phage (such as a Myoviridae phage. Tevenvirinae phage or Tequatrovirus phage)), adeno-associated virus (AAV), herpes simplex virus, retrovirus or lentivirus.

Caudovirales is an order of viruses known as the tailed bacteriophages. Each virus may be a Caudovirales phage, eg, a Ackermannviridae, Autographiviridae, Chaseviridae, Demerecviridae, Drexlerviridae, Herelleviridae, Myoviridae, Podoviridae, Siphoviridae or Lilyvirus phage. Optionally, in this case the heterologous DNA encodes a tail fibre or component thereof. The heterologous DNA may further encode a Cas and/or crRNA or gRNA as disclosed herein.

Each virus may be a T-even phage. Both the first and second viruses may be the same type of T-even phage, eg, both are a T4 phage.

T-even phages are in fact among the largest and highest complexity virus, in which these phages genetic information is made up of around 160 genes. Coincident with their complexity, T-even viruses were found to have the presence of the unusual base hydroxymethylcytosine (HMC) in place of the nucleic acid base cytosine. In addition to this, the EMC residues on the T-even phage are glucosylated in a specific pattern. Another unique feature of the T-even virus is its regulated gene expression. These unique features and other features gave significance of the T-even phages, this includes transduction which is responsible for transfer of drug resistant features, lysogenic conversion is responsible for acquisition of new characteristics such as the formation of new enzymes, random insertion into bacterial chromosome can induce insertional mutation, epidemiological typing of bacteria (phage typing), phages are used extensively in genetic engineering where they serve as cloning vectors. The T4 virus's double-stranded DNA genome is about 169 kbp long and encodes 289 proteins. The T4 genome is terminally redundant and is first replicated as a unit, then several genomic units are recombined end-to-end to form a concatemer. When packaged, the concatemer is cut at unspecific positions of the same length, leading to several genomes that represent circular permutations of the original. The T4 genome bears eukaryote-like intron sequences. Escherichia virus T4 is a species of bacteriophages that infect Escherichia coli bacteria. It is a double-stranded DNA virus in the subfamily Tevenvirinae from the family Myoviridae. T4 is capable of undergoing only a lytic lifecycle and not the lysogenic lifecycle. The species was formerly named T-even bacteriophage, a name which also encompasses includes among other strains (or isolates) including Enterobacteria phage T2, Enterobacteria phage T4 and Enterobacteria phage T6. Enterobacteria phage T2 is a virus that infects and kills E. coli. It is in the genus Tequatrovirus, and the family Myoviridae. Its genome consists of linear double-stranded DNA, with repeats at either end. The phage is covered by a protective protein coat. Tequatrovirus is a genus of viruses in the order Caudovirales, in the family Myoviridae, in the subfamily Tevenvirinae. The T2 phage can quickly turn an E. coli cell into a T2-producing factory that releases phages when the cell ruptures. Enterobacteria phage T6 is a bacteriophage strain that infects Escherichia coli bacteria. It was one bacteriophage that was used as a model system in the 1950s in exploring the methods viruses replicate, along with the other T-even bacteriophages comprising Enterobacteria phage T2, Enterobacteria phage T4 and Enterobacteria phage T2.

The inventors analysed the genomes of several phages, as follows, which were found to contain dispensable parts of their genomes, ie, DNA that can be deleted to create space for heterologous DNA. See, for example, SEQ ID NOs: 1-128. In an example, each virus may, thus, be a phage selected from the group consisting of Escherichia phage T4, Escherichia phage T2, Escherichia phage T6,m Escherichia phage RB69, Shigella phage Shf125875, Escherichia phage APCEc01, Escherichia phage moskry, Escherichia phage ST0, Escherichia phage vB_EcoM_JS09, Shigella phage SP18, Escherichia phage vB_EcoM_PhAPEC2, Escherichia phage HX01, Salmonella phage SG1, Shigella phage pSs-1, Escherichia phage HY01, Yersinia phage PST, Escherichia phage AR1, Escherichia phage phiE142, Shigella phage SHFML-11, Escherichia phage slur07, Shigella phage SHFML-11, Escherichia phage UFV-AREG1, Escherichia phage vB_EcoM-UFV13, Shigella phage JK38, Shigella phage SHFML-26, Shigella phage Sf22, Escherichia phage ime09, Shigella phage SH7, Yersinia phage phiD1, Escherichia phage RB3, Escherichia phage ECML-134, Escherichia phage vB_EcoM_ACG-C40, Escherichia phage vB_EcoM-fFiEco16, Escherichia phage PP01, Shigella phage Shfl2, Escherichia phage ECO4, Escherichia virus RB14, Escherichia phage vB_EcoM_JB75, Shigella phage Sf22, Escherichia phage vB_vPM_PD112, Shigella phage Sf23, Escherichia phage vB_EcoM_G2540, Escherichia phage vB_EcoM_G2133, Escherichia phage vB_EcoM_G4498, Escherichia virus RB32, Escherichia phage vB_EcoM_G4507, Escherichia phage vB__EcoM_G8, Escherichia phage EcNP 1, Enterobacteria phage RB27, Shigella virus KRT47, Escherichia phage teqdroes, Escherichia phage slur02, Yersinia phage fPS-90, Yersinia phage phiD1, Shigella phage Sf24 and Escherichia phage phiC120. Both the first and second viruses may be the same type of phage selected from said group.

Each virus may be a phage and the hybrid DNA excludes a DNA sequence that is comprised by a gene of the first virus genome, wherein the gene encodes an amino acid sequence selected from SEQ ID Nos: 1-128, or a homologue thereof; optionally wherein the homologue is an amino acid sequence that is at least 80, 85, 90, 91, 92, 93, 94, 95, 96, 97, 98 or 99% identical to said selected sequence (preferably at least 90% identical).

The hybrid DNA may exclude a plurality of DNA sequences of the first virus genome, wherein each DNA sequence is comprised by a respective gene of the first virus genome, wherein the gene encodes an amino acid sequence selected from SEQ ID Nos: 1-128, or a homologue thereof; optionally wherein the homologue is an amino acid sequence that is at least 80, 85, 90, 91, 92, 93, 94, 95, 96, 97, 98 or 99% identical to said selected sequence (preferably at least 90% identical).

The hybrid DNA may exclude one or more DNA sequences of the first virus genome, wherein each DNA sequence is comprised by a respective gene of the first virus genome, wherein the gene encodes an amino acid sequence selected from SEQ ID Nos: 1-42, or a homologue thereof; optionally wherein the homologue is an amino acid sequence that is at least 80, 85, 90, 91, 92, 93, 94, 95, 96, 97, 98 or 99% identical to said selected sequence (preferably at least 90% identical).

Each virus may be a phage (such as a T even phage) and the hybrid DNA excludes one or more DNA sequences of the first virus genome, wherein each DNA sequence is comprised by at least 10, 20, 30, 40, 50, 60, 70, 80 or 90% (or 100%) of a respective gene of the first virus genome, wherein (i) the gene encodes an amino acid sequence selected from SEQ ID Nos: 1-42, or a homologue thereof, optionally wherein the homologue is an amino acid sequence that is at least 80, 85, 90, 91, 92, 93, 94, 95, 9%, 97, 98 or 99% identical to said selected sequence (preferably at least 90% identical); or (ii) the gene is selected from T4 phage genes 49.1, 49.2, 49.3, nrdC, nrdC.1, nrdC.2, nrdC.3, nrdC.4, nrdC.5, nrdC 6, nrdC.7, nrdC.8, nrdC.9, nrdC 10, nrdC.11, mobD, mobD.1, mobD.2, mobD.2a, mobD.3, mobD.4, mobD.5, rL-1, rI, rI.1, tk, tk.1, tk.2, tk.3, tk.4, vs, vs.1, regB, vs.3, vs.4, vs.5, vs.6, vs.7, vs.8, denV, IpIII and IpII; or an orthologue or homologue thereof. The homologue comprises a DNA sequence that is at least 70, 80, 85, 90, 95, 96, 97, 98 or 99% identical to the gene. The genes of (ii) were found to be dispensable by the inventors' analysis.

The hybrid DNA may excludes one or more genes of the first virus genome, wherein each gene is selected from T4 phage genes 49.1, 49.2, 49.3, nrdC, nrdC.1, nrdC.2, nrdC.3, nrdC.4, nrdC.5, nrdC.6, nrdC.7, nrdC.8, nrdC.9, nrdC.10, nrdC.11, mobD, mobD.1, mobD.2, mobD.2a, mobD.3, mobD.4, mobD.5, rL-1, rI, rI.1, tk, tk.1, tk.2, tk.3, tk.4, vs, vs.1, regB3, vs.3, vs.4, vs.5, vs.6, vs.7, vs.8, denV, IpIII and IpII; or an orthologue or homologue thereof. The homologue comprises a DNA sequence that is at least 70, 80, 85, 90, 95, 96, 97, 98 or 99% identical to the gene.

The hybrid DNA may exclude DNA from 2 or mom genes of the first virus genome. For example, the hybrid DNA excludes 2-50, 2-40, 2-30, 2-20, 2-10 or 2-5 genes of the first virus genome.

The inventors' analysis also found that genes encoding certain protein types may be dispensable, and thus DNA comprised by one or mote of such genes can be deleted from the virus genome to make space for the heterologous DNA. In an example, each gene may, thus, encode a protein selected from a thioredoxin, endonuclease (optionally a homing endonuclease, a RegB site-specific RNA endonuclease or a site-specific intron-like DNA endonuclease), lysis inhibition regulator, membrane protein, thymidine kinase, protein that contains a A1pp phosphatase motif, tRNA synthetase modifier (optionally a valyl-tRNA synthetase modifier), mRNA processing protein, U V repair enzyme (optionally a N-glycosylase UV repair enzyme), internal head protein (eg, a IpIII internal head protein or a IpII internal head protein, Ip4 protein), endoribonuclease and DNA glycosylase (optionally a pyrimidine dimer DNA glycosylase).

The second virus may comprise a capsid that has a DNA packaging capacity that is from 90-110% of the packaging capacity (optionally the same packaging capacity) of the first virus.

The hybrid DNA may be 90-110% the size (eg, the same size) of the DNA of the first virus genome.

The second virus may comprise a capsid that has a DNA packaging capacity of Zbp and the total number of base pairs of the hybrid DNA is 90-110% (eg, 100%) of Z.

The size of the first virus genome may be 90-100% (eg. 100%) of Z; and/or the size of the first virus genome is smaller than Z by 5-50% (eg, 5-40, 5-30, 5-20 or 5-10%) of X.

The first and second viruses may have the same DNA packaging capacity.

Each virus may comprise a life cycle having a lytic pathway, optionally wherein (i) each virus is a lytic virus (eg, each is a lytic phage); or (ii) the first virus is a temperate virus (eg, phage) having a life cycle comprising a lytic pathway and a lysogenic pathway, wherein the second virus (eg, phage) has a life cycle comprising a lytic pathway but no lysogenic pathway or a disrupted lysogenic pathway wherein the second virus has a reduced chance of entering a lysogenic pathway than the first virus. Alternatively, each virus is a non-lytic virus (eg, non-lytic phage).

There is provided:—

A synthetic phage, wherein the phage is
(a) a synthetic T4 phage comprising a deletion of DNA from a Deletion Permissive Region (DPR) of the genome of the phage, wherein the region is between the pin (protease inhibitor) gene and the iPII (internal protein) gene; or
(b) a synthetic version of a phage that is not a T4 phage, wherein the synthetic phage comprises a deletion of DNA from a region of its genome that is homologous or orthologous to said DPR; and
wherein the synthetic phage is capable of replication in a host cell.

The deletion may comprise up to 8000 bp of DNA, eg, the deletion may comprise from 50-800, 50-700, 50-600, 50-500, 50-400, 50-300, 50-200, 50-100, 100-800, 100-700, 100-600, 100-500, 100-400, 100-300, 100-200, 150-800, 150-700, 150-600, 150-500, 150-400, 150-300, 150-200, 200-800, 200-700, 200-600, 200-500, 200-400, 200-300, 300-800, 300-700, 300-600, 300-500, 300-400, 400-800, 400-700, 400-600, 400-500, 500-800, 500-700, 500-600, 600-800 or 600-700 bp of DNA. The deletion may comprise up to 1, 2, 3, 4, or 5 kb of DNA.

The synthetic phage of (i) may comprise an insertion of heterologous DNA, wherein the insertion is between the pin gene and the ipII gene, or the synthetic phage of (ii) comprises an insertion of heterologous DNA, wherein the insertion is between a first gene and a second gene, wherein the first gene is homologous or orthologous to the pin gene of T4 and the second gene is homologous or orthologous to the ipII gene of T4.

The insertion may comprise up to 8000 bp of DNA, eg, the insertion may comprise from 50-800, 50-700, 50-600, 50-500, 50-400, 50-300, 50-200, 50-100, 100-800, 100-700, 100-600, 100-500, 100-400, 100-300, 100-200, 150-800, 150-700, 150-600, 150-500, 150-400, 150-300, 150-200, 200-800, 200-700, 200-600, 200-500, 200-400, 200-300, 300-800, 300-700, 300-600, 300-500, 300-400, 400-800, 4(0)-700, 400-600, 400-500, 500-800, 500-700, 500-600, 600-800 or 600-700 bp of DNA. The insertion may comprise up to 1, 2, 3, 4, or 5 kb of DNA

The insertion may comprise a total number (X) of base pairs of heterologous DNA, and (a) the deletion comprises a total number (Y) of base pairs of DNA wherein Y is at least 50% of X; or (b) the T4 phage or said phage that is not a T4 comprises a capsid that has a DNA packaging capacity of Zbp and the total number of base pairs of the genomic DNA of the synthetic phage is 90-110% of Z. X may be a value of X disclosed herein. Y may be a value of Y disclosed herein. Z may be a value of Z disclosed herein

There is provided:—

A synthetic phage, wherein the phage is
(a) a synthetic T4 phage comprising an insertion of heterologous DNA into a Deletion Permissive Region (DPR) of the genome of the phage, wherein the region is between the pin (protease inhibitor) gene and the ipII (internal protein) gene; or
(b) a synthetic version of a phage that is not a T4 phage, wherein the synthetic phage comprises an insertion of heterologous DNA into a region of its genome that is homologous or orthologous to said DPR; and
   wherein the synthetic phage is capable of replication in a host cell.

The insertion may comprise up to 8000 bp of DNA, eg, the insertion may comprise from 50-800, 50-700, 50-600, 50-500, 50-400, 50-300, 50-200, 50-100, 100-800, 100-700, 100-600, 100-500, 100-400, 100-300, 100-200, 150-800, 150-700, 150-600, 150-500, 150-400, 150-300, 150-200, 200-800, 200-700, 200-600, 200-500, 200-400, 200-300, 300-800, 300-700, 300-600, 300-500, 300-400, 400-800, 400-700, 400-600, 400-500, 500-800, 500-700, 500-600, 600-800 or 600-700 bp of DNA. The insertion may comprise up to 1, 2, 3, 4, or 5 kb of DNA.

In any configuration, the DPR of the T4 phage may comprise contiguous DNA between the pin gene and the ipII gene, wherein the contiguous DNA is at least 100, 200, 300, 400, 500, 600, 700, 800, 900 or 1000 bp in length; or wherein the DPR of the T4 phage comprises at least 100 bp of DNA between the pin gene and the ipII gene. The contiguous DNA may be no more than or 1000, 2000, 3000, 4000 or 5000 bp in length.

The DPR of the T4 phage may extend from the pin gene to the ipII gene.

The DPR of the T-even phage may comprise or consist of DNA (i) between T4 genome coordinates 2625 and 8092; 2668 and 7178; 8643 and 10313; 9480 and 12224; or 9067 and 16673; or (ii) between homologous coordinates wherein said phage is a non-T4 phage that is a T-even phage. The T4 genome of (i) may comprise or consist of SEQ ID NO: 129.

In an example,
A. the synthetic phage genome comprises a deletion of a one or more genes, wherein each gene encodes a protein selected from a thioredoxin, endonuclease (optionally a homing endonuclease, a RegB site-specific RNA endonuclease or a site-specific intron-like DNA endonuclease), lysis inhibition regulator, membrane protein, thymidine kinase, protein that contains a A1pp phosphatase motif, tRNA synthetase modifier (optionally a valyl-tRNA synthetase modifier), mRNA processing protein, UV repair enzyme (optionally a N-glycosylase UV repair enzyme), internal head protein (eg, a ipIII internal head protein or a ipII internal head protein, Ip4 protein), endoribonuclease and DNA glycosylase (optionally a pyrimidine dimer DNA glycosylase);
B. the synthetic phage genome comprises a deletion of one, more or all T4 genes of Table 7, or homologues or orthologues thereof;
C. the synthetic phage genome comprises a deletion of T4 gene(s) (a) nrdC, (b) mobD, (c) rI, (d) rI.1, (e) tk, (f) vs, (g) regB and/or (h) denV, or a homologue or orthologue thereof; or
D. the synthetic phage genome comprises a deletion of DNA between coordinates
a) 2625 and 8092;
b) 2668 and 7178;
c) 8643 and 10313; or
d) 9480 and 12224
wherein the coordinates are the nucleotide positions in the direction from the pin gene towards the mobD and iPII genes of T4; or wherein homologous DNA from a T-even phage is deleted wherein said T-even phage is not a T4 phage.

The synthetic phage genome may comprise a deletion of T4 genes tk, vs and regB, or homologues or orthologues thereof; optionally a deletion of DNA stretching from T4 gene nrdC to denV, or homologues or orthologues thereof.

The synthetic phage genome may comprise a deletion of one or more genes, wherein
A. each gene encodes a protein comprising an amino acid sequence selected from SEQ ID Nos: 1-128, or a homologue thereof; optionally wherein the homologue is an amino acid sequence that is at least 80% identical to said selected sequence; and/or
B. each gene encodes an amino acid sequence selected from SEQ ID Nos: 1-42, or a homologue thereof; optionally wherein the homologue is an amino acid sequence that is at least 80% identical to said selected sequence.

The synthetic phage of (ii) may be a T-even phage.

The synthetic phage may be a lytic phage; and/or said phage that is not a T4 phage is a lytic phage.

There is provided:—

A DNA comprising the genome of the synthetic phage; optionally wherein the DNA is a chromosome of a bacterial cell or an episome (eg, a plasmid) comprised by a bacterial cell, such as a host cell of said synthetic phage.

The heterologous DNA may comprise or encode
A. one or more components of a CRISPR/Cas system or a guided nuclease (eg, a Cas, TALEN, meganuclease or zinc finger); optionally wherein the heterologous DNA encodes a guide RNA (eg, a single guide RNA) and/or a Cas (eg, a Cas9, Cas3, Cas12, Cas13 or Cas14);
B. an antibacterial agent;
C. a phage tail fibre or component thereof;
D. a vitamin;
E. a blood protein;
F. an antibody or fragment thereof; or
G. a human or plant protein or fragment thereof.

The phage that is not a T4 phage may be selected from the group consisting of the phages of Table 6, Escherichia phage T4, Escherichia phage T2, Escherichia phage T6, Escherichia phage RB69, Shigella phage Shf125875, Escherichia phage APCEc01, Escherichia phage moskry, Escherichia phage ST0, Escherichia phage vB_EcoM_JS09, Shigella phage SP18, Escherichia phage vB_EcoM_PhAPEC2, Escherichia phage HX01, Salmonella phage SG1, Shigella phage pSs-1, Escherichia phage HY01, Yersinia phage PST, Escherichia phage AR1, Escherichia phage phiE142, Shigella phage SHFML-11, Escherichia phage slur07, Shigella phage SHFML-11, Escherichia phage UFV-AREG1, Escherichia phage vB_EcoM-UFV13, Shigella phage JK38, Shigella phage SHFML-26, Shigella phage Sf22, Escherichia phage ime09, Shigella phage S17, Yersinia phage phiD1, Escherichia phage RB3, Escherichia phage ECML-134, Escherichia phage vB_EcoM_ACG-C40, Escherichia phage vB_EcoM-fFiEco6, Escherichia phage PP01, Shigella phage Shfl2, Escherichia phage ECO4, Escherichia virus RB14, Escherichia phage vB_EcoM_JB75, Shigella phage Sf22, Escherichia phage vB_vPM_PD112, Shigella phage Sf23, Escherichia phage vB_EcoM_G2540, Escherichia phage vB_EcoM_G2133, Escherichia phage vB_EcoM_G4498, Escherichia virus RB32, Escherichia phage vB_EcoM_G4507, Escherichia phage vB_EcoM_G8, Escherichia phage EcNP 1, Enterobacteria phage RB27, Shigella virus KRT47, Escherichia phage teqdroes, Escherichia phage slur02, Yersinia phage fPS-90, Yersinia phage phiD1, Shigella phage Sf24 and Escherichia phage phiC120.

There is provided:—

A method of producing synthetic phage particles, comprising
(a) Allowing the production of synthetic phage in producer cells, wherein the phage are according to the invention; and
(b) Isolating the phage; and
(c) Optionally combining a population of said isolated synthetic phage with a pharmaceutically acceptable excipient, carrier or diluent to produce a pharmaceutical composition.

There is provided:—

A method of producing a pharmaceutical composition, the method comprising combining a population of synthetic phage with a pharmaceutically acceptable excipient, carrier or diluent to produce a pharmaceutical composition, wherein the phage are according to the invention.

There is provided:—

A population of synthetic phage according to the invention, or a pharmaceutical composition obtainable by the method of the invention, for use as a medicament: optionally for administration to a human or animal subject for reducing infection by pathogenic host bacterial or archaeal cells or a first species or strain, wherein the phage are capable of infecting cells of said species or strain.

There is provided:—

A synthetic phage, wherein the phage is
(a) a synthetic Phi92 phage comprising a deletion of DNA from a Deletion Permissive Region (DPR) of the genome of the phage, wherein the region is between gene 39 and gene 46 or between gene 230 and gene 240; or
(b) a synthetic version of a phage that is not a Phi92 phage, wherein the synthetic phage comprises a deletion of DNA from a region of its genome that is homologous or orthologous to said DPR; and
   wherein the synthetic phage is capable of replication in a host cell.

The deletion may comprise up to 8000 bp of DNA, eg, the deletion may comprise from 50-800, 50-700, 50-600, 50-500, 50-400, 50-300, 50-200, 50-100, 100-800, 100-700, 100-600, 100-500, 100-400, 100-300, 100-200, 150-800, 150-700, 150-600, 150-500, 150-400, 150-300, 150-200, 200-800, 200-700, 200-600, 200-500, 200-400, 200-300, 300-800, 300-700, 300-600, 300-500, 300-400, 400-800, 400-700, 400-600, 400-500, 500-800, 500-700, 500-600, 600-800 or 600-700 bp of DNA. The deletion may comprise up to 1, 2, 3, 4, or 5 kb of DNA.

The synthetic phage of (i) may comprise an insertion of heterologous DNA, wherein the insertion is between genes 39 and 46 or between genes 230 and 240, or the synthetic phage of (ii) comprises an insertion of heterologous DNA, wherein the insertion is between a first gene and a second gene; wherein the first gene is homologous or orthologous to gene 39 of Phi92 and the second gene is homologous or orthologous to gene 46 of Phi92, or wherein the first gene is homologous or orthologous to gene 230 of Phi92 and the second gene is homologous or orthologous to gene 240 of Phi92.

The insertion may comprise a total number (X) of base pairs of heterologous DNA, and (a) the deletion comprises a total number (Y) of base pairs of DNA wherein Y is at least 50% of X; or (b) the Phi92 phage or said phage that is not a Phi92 comprises a capsid that has a DNA packaging capacity of Zbp and the total number of base pairs of the genomic DNA of the synthetic phage is 90-110% of Z. X may be a value of X disclosed herein. Y may be a value of Y disclosed herein. Z may be a value of Z disclosed herein.

There is provided:—

A synthetic phage, wherein the phage is
(a) a synthetic Phi92 phage comprising an insertion of DNA into a Deletion Permissive Region (DPR) of the genome of the phage, wherein the region is between gene 39 and gene 46 or between gene 230 and gene 240; or
(b) a synthetic version of a phage that is not a Phi92 phage, wherein the synthetic phage comprises an insertion of DNA into a region of its genome that is homologous or orthologous to said DPR; and
   wherein the synthetic phage is capable of replication in a host cell.

The insertion may comprise up to 8000 bp of DNA, eg, the insertion may comprise from 50-800, 50-700, 50-600, 50-500, 50-400, 50-300, 50-200, 50-100, 100-800, 100-700, 100-600, 100-500, 100-400, 100-300, 100-200, 150-800, 150-700, 150-600, 150-500, 150-400, 150-300, 150-200, 200-800, 200-700, 200-600, 200-500, 200-400, 200-300, 300-800, 300-700, 300-600, 300-500, 300-400, 400-800, 400-700, 400-600, 400-500, 500-800, 500-700, 500-600, 600-800 or 600-700 bp of DNA. The insertion may comprise up to 1, 2, 3, 4, or 5 kb of DNA.

The DPR of the Phi92 phage may comprise contiguous DNA between gene 39 and gene 46 or between gene 230 and gene 240, wherein the contiguous DNA is at least 1000 bp in length; or wherein the DPR of the Phi92 phage comprises at least 100 bp of DNA between gene 39 and gene 46 or between gene 230 and gene 240

The DPR of the Phi92 phage may extends from gene 39 to gene 46 and/or from gene 230 to gene 240.

In an example,
A. the synthetic phage genome comprises a deletion of a one or more genes, wherein each gene encodes a DNA methylase; and/or
B. the synthetic phage genome comprises a deletion of one, more or all Phi92 genes of Table 9, or homologues or orthologues thereof.

The synthetic phage genome may comprise
(a) a deletion in one or more Phi92 genes 235, 236, 237, 238, 239 and 240, or homologues or orthologues thereof; optionally a deletion of DNA stretching from genes 235-240 or 238-240, or homologues or orthologues thereof; or
(b) a deletion of Phi92 genes 39-46 and/or 235-240, or homologues or orthologues thereof.

The synthetic phage of (ii) may be a rV5 or a rV5-like phage.

The synthetic phage may be a lytic phage; and/or said phage that is not a Phi92 phage is a lytic phage.

There is provided:—

A DNA comprising the genome of the synthetic phage; optionally wherein the DNA is a chromosome of a bacterial cell or an episome (eg, a plasmid) comprised by a bacterial cell, such as a host cell of said synthetic phage.

The heterologous DNA may comprise or encode
A. one or more components of a CRISPR/Cas system or a guided nuclease (eg, a Cas, TALEN, meganuclease or zinc finger); optionally wherein the heterologous DNA encodes a guide RNA (eg, a single guide RNA) and/or a Cas (eg, a Cas9, Cas3, Cas12, Cas13 or Cas14);
B. an antibacterial agent;
C. a phage tail fibre or component thereof;
D. a vitamin;
E. a blood protein;
F. an antibody or fragment thereof; or
G. a human or plant protein or fragment thereof.

There is provided:—

A method of producing synthetic phage particles, comprising
(a) Allowing the production of synthetic phage in producer cells, wherein the phage are according to the invention; and
(b) Isolating the phage; and
(c) Optionally combining a population of said isolated synthetic phage with a pharmaceutically acceptable excipient, carrier or diluent to produce a pharmaceutical composition.

There is provided:—

A method of producing a pharmaceutical composition, the method comprising combining a population of synthetic phage with a pharmaceutically acceptable excipient, carrier or diluent to produce a pharmaceutical composition, wherein the phage are according to the invention.

There is provided:—

A population of synthetic phage according to the invention, or a pharmaceutical composition obtainable by the method of claim 18, for use as a medicament: optionally for administration to a human or animal subject for reducing infection by pathogenic host bacterial or archaeal cells or a first species or strain, wherein the phage are capable of infecting cells of said species or strain.

There is provided:—

A method of producing synthetic virus particles, comprising (i) carrying out the method described herein to produce the hybrid DNA, (ii) introducing the hybrid DNA into a target cell of a first species or strain in which the hybrid DNA is capable of being replicated and particles of said second virus are produced; and (iii) producing second viruses in the cell, and (iv) further optionally isolating second virus particles from the cell.

The method may be carried out using a plurality of target cells, wherein hybrid DNA is introduced into the cells and a plurality of second virus particles are produced, and optionally isolating said plurality of particles.

The method may comprise, further producing a pharmaceutical composition comprising second virus particles obtained by step (iv) and a pharmaceutically acceptable excipient, carrier of diluent.

The method may further comprise producing a composition comprising second virus particles obtained by step (iii) or (iv) and an excipient, carrier of diluent.

There is provided a method of producing a composition, the method comprising combining a plurality of second virus particles obtainable by the method with an excipient, carrier of diluent.

There is provided a method of producing a pharmaceutical composition, the method comprising combining a plurality of second virus particles obtainable by the method with a pharmaceutically acceptable excipient, carrier of diluent.

There is provided:—

A method of selecting a synthetic virus, the method comprising
(a) Providing a first type (T1) of a virus, wherein the virus is obtained or obtainable by the method of described herein of producing a modified genome;
(b) Providing a second type (T2) of a virus, wherein the virus is obtained or obtainable by the method of described herein of producing a modified genome, wherein T1 and T2 differ from each other by at least said heterologous DNA comprised by each type (eg, T1 and T2 differ by heterologous DNA encoding first and second tail fibres respectively, wherein the tail fibres are different);
(c) Culturing the T1 virus with target cells of the first species or strain; and culturing the T2 virus with target cells of the first species or strain;
(d) Determining which of the cultured T1 and T2 viruses produces a predetermined indicator or the extent of production of the indicator by the viruses.
(e) Selecting T1 or T2 virus on the basis of the determination in step (d); and
(f) Optionally further producing further copies of the selected virus and/or determining the sequence of the heterologous DNA or a portion thereof comprised by the selected virus.

Step (c) may comprise separately culturing T1 and T2 In an alternative, step (c) comprises culturing T1 and T2 together. Preferably, the viruses are cultured under identical (or substantially identical) conditions. As will be apparent to the skilled addressee, relevant conditions may be selected from culture time, culture temperature, culture medium, pfu (plaque forming units) for virus and host cell cfu (colony forming units) at the start of culture.

The indicator may be virus titre (ie, the titre of T1 viruses is determined, and the titre of T2 viruses is determined). As the skilled addressee knows, titre may be determined as the number of plaque forming units per unit volume (eg, pfu per ml or microlitre) as determined by routine methods. The indicator may be the extent of colony formation when the viruses are contacted with a lawn of host cells and incubated. The indicator may be expression of a protein or RNA encoded by the heterologous DNA. The indicator may be host cell killing or the extent of host cell killing. Cells may be bacterial or archaeal cells, for example.

T1 viruses may be capable of infecting target cells in step (c), but T2 viruses are not capable of infecting of target cells in step (c) or are less infective than T1 viruses; wherein step (d) comprises determining the extent of target cell infectivity of each of T1 and T2, optionally by determining the titres of T1 and T2 viruses that have been cultured.

The method may be carried out using at least 2, 3, 4, 5, 6, 7, 8, 9, 10 or 20 (eg, at least 3; or at least 4; or at least 5) different types of second virus, wherein the types differ from each other by their said heterologous DNAs (optionally wherein the types comprise DNA encoding different tail fibres).

There is provided:—

A virus infectivity assay, the assay comprising
(a) providing a first type (T1) of virus comprising a first DNA sequence;
(b) providing a second type (T2) of virus comprising a second DNA sequence, wherein T1 and T2 differ from each other by said DNA sequences (preferably, T1 and T2 only differ from each other by said DNA sequences) and differ in infectivity of target cells;
(c) Culturing the T1 virus with target cells of a first species or strain; and culturing the T2 virus with target cells of the first species or strain;
(d) Determining which of the cultured T1 and T2 viruses produces a predetermined indicator or the extent of production of the indicator by the viruses;
(e) Selecting T1 or T2 virus on the basis of the determination in step (d); and
(f) Optionally further producing further copies of the selected virus and/or determining the sequence of said DNA or a portion thereof comprised by the selected virus.

Step (d) may comprise separately culturing T1 and T2. In an alternative, step (c) comprises culturing T1 and T2 together. Preferably, the viruses are cultured under identical (or substantially identical) conditions. As will be apparent to the skilled addressee, relevant conditions may be selected from culture time, culture temperature, culture medium, pfu (plaque forming units) for virus and host cell cfu (colony forming units) at the start of culture.

The indicator may be virus titre (ie, the titre of T1 viruses is determined, and the titre of T2 viruses is determined). As the skilled addressee knows, titre may be determined as the number of plaque forming units per unit volume (eg, pfu per ml or microlitre) as determined by routine methods. The indicator may be the extent of colony formation when the viruses are contacted with a lawn of host cells and incubated. The indicator may be expression of a protein or RNA encoded by the heterologous DNA The indicator may be host cell killing or the extent of host cell killing. Cells may be bacterial or archaeal cells, for example.

T1 viruses may be capable of infecting target cells in step (c), but T2 viruses are not capable of infecting of target cells in step (c) or are less infective than T1 viruses; wherein step (d) comprises determining the extent of target cell infectivity of each of T1 and T2, optionally by determining the titres of T1 and T2 viruses that have been cultured.

The method may be carried out using at least 2, 3, 4, 5, 6, 7, 8, 9, 10 or 20 (eg, at least 3; or at least 4; or at least 5) different types of second virus, wherein the types differ from each other by their said heterologous DNAs (optionally wherein the types comprise DNA encoding different tail fibres).

The T1 virus may be capable of infecting target cells in step (c), but T2 virus is not capable of infecting of target cells in step (c) or is less infective than T1 virus; wherein step (d) comprises determining the extent of target cell infectivity of each of T1 and T2, optionally by determining the titres of T1 and T2 viruses that have been cultured.

T1 and T2 may differ only by DNA sequences encoding first and second viral tail fibres respectively, wherein the tail fibres are different.

The T1 and T2 viruses may differ only by their tail fibres.

There is provided:—

A method of producing a composition comprising synthetic virus particles, the method comprising obtaining particles of a first type from a culture and optionally combining the obtained particles with an excipient, carrier or diluent, wherein the culture comprises target cells, each cell comprising DNA comprising the genome of the first type of virus, wherein the virus comprises the sequence determined in step (f).

A method of producing synthetic virus particles, the method comprising
(a) culturing target cells, each cell comprising DNA comprising the genome of a first type of virus, wherein the virus comprises the sequence determined in step (f);
(b) producing virus particles in the cells,
(c) obtaining virus particles from the cell culture and
(d) optionally combining the obtained particles with a pharmaceutically acceptable excipient, carrier or diluent to produce a pharmaceutical composition.

Any composition (eg, a pharmaceutical composition) herein may be comprised by a sterile container or medical container, eg, a syringe, IV bag, autoinjector pen or a vial.

A composition or second virus(es) herein may be for use as a medicine, or for medical use.

A composition or second virus(es) herein may be for administration to a human or animal subject for treating or preventing a disease or condition in the subject, wherein the disease or condition is caused by or associated with target cells, wherein the second viruses are capable of infecting and killing target cells.

A composition or second virus(es) herein may be for administration to a human or animal subject for treating a disease or condition in the subject, wherein the disease or condition is associated with target cells, wherein the second viruses are capable of infecting and killing target cells.

Thus, compositions and viruses of the invention may be used for human or animal therapy. In an embodiment (as exemplified in Example 1), it may be advantageous to retain one or more virus genes in the viral genome, wherein each of those genes is for DNA modification or host DNA degradation.

Killing of target cells comprised by a cell population of the subject may be beneficial where the cells are undesirable (eg, detrimental to the health of a subject to which the method is applied, or detrimental to an ex vivo environment or in vitro cell sample to which the method is applied or the composition is administered).

A composition or second virus(es) herein may be for killing target cells comprised by an environment, wherein the second viruses are capable of infecting and killing target cells.

The hybrid DNA may encode a first CRISPR/Cas system to modify a first protospacer of the genome of target cells. The hybrid DNA may further encode a second CRISPR/Cas system to modify a second protospacer of the genome, wherein the second protospacer is different to the first protospacer.

The method of infecting target cells may be carried out in vitro or in vivo. #

For example the target cell(s) is an E coli, Enterococcus, Enterobacteriaciae, Colstridum (eg, C difficile), Kelbsiella (eg, K pneumoniae), Pseudomonas (eg, P aeruginosa or syringae) or Staphylococcus (eg, S aureus) cell. For example the target cell(s) is a cell of a genus or species disclosed in Table 1.

The hybrid DNA may encode a plurality of crRNAs, wherein each said crRNA is encoded by a CRISPR array comprising first and second repeat sequences and a spacer sequence joining the repeat sequences. In an example each repeat sequence is

(SEQ ID NO: 138)
GAGTTCCCCGCGCCAGCGGGGATAAACCG
or
(SEQ ID NO: 139)
GTTTTATATTAACTAAGTGGTATGTAAAT.

In an example, each protospacer or spacer sequence consists of from 15 to 70, 20 to 50, 17 to 45, 18 to 40, 18 to 35 or 20 to 40 contiguous nucleotides.

Optionally, Cas1 and/or Cas2 are not encoded by the hybrid DNA. Optionally, Cas4 is not encoded by the hybrid DNA.

The hybrid DNA may comprise nucleotide sequences encoding a type I Cas3 and Cascade proteins each under the control of a constitutive promoter. The Cas3 may be a Type-TB Cas3 or a Type-IE Cas3 or a Type-IF Cas3. The hybrid DNA may encode a Cas disclosed in WO2019002218 and optionally a crRNA that is encoded by a CRISPR array comprising cognate repeat sequences. All of these disclosures in WO2019002218 are expressly incorporated herein by reference for possible use in the present invention.

The hybrid DNA may encode a first Cas (C1) and/or a second Cas (C2), wherein
(a) C1 is a Class 1 Cas and C2 is a Class 1 Cas;
(b) C1 is a Class 1 Cas and C2 is a Class 2 Cas;
(c) C1 is a Class 2 Cas and C2 is a Class 2 Cas;
(d) C1 is a Type I Cas (optionally Type I-A, B, C, D, E, F or U) and C2 is a Type I Cas (optionally Type I-A, B, C, D, E, F or U);
(e) C1 is a Type I (optionally Type I-A, B, C, D, E, F or U) or II Cas and C2 is a Type II Cas;
(f) C1 is a Type I (optionally Type I-A, B, C, D, E, F or U) or II Cas and C2 is a Type Ill Cas (optionally Type I-A or B);
(g) C1 is a Type I (optionally Type I-A, B, C, D, E, F or U) or II Cas and C2 is a Type IV Cas;
(h) C1 is a Type I (optionally Type I-A, B, C, D, E, F or U) or II Cas and C2 is a Type V Cas; or
(i) C1 is a Type I or 11 Cas and C2 is a Type VI Cas.

Optionally, C1 is a Type I-A, B, C, D, E, F or U Cas. Optionally, C2 is a Type I-A, B, C, D, E, F or U Cas Optionally, C1 is a Type I-A Cas and C2 is a Type I-B, C, E, F or U Cas. Optionally, C1 is a Type I-B Cas and C2 is a Type I-B, C, F, F or U Cas. Optionally, C1 is a Type I-C Cas and C2 is a Type I-B, C, E, F or U Cas. Optionally, C1 is a Type I-D Cas and C2 is a Type I-B, C, E, F or U Cas. Optionally, C1 is a Type I-E Cas and C2 is a Type I-B, C, E, F or U Cas. Optionally, C1 is a Type I-F Cas and C2 is a Type I-B, C, E, F or U Cas. Optionally, C1 is a Type I-U Cas and C2 is a Type I-B, C, E, F or U Cas.

Optionally,
(a) C1 is a Type IB or C Cas and C2 is a Type I-E or F Cas (optionally C1 is a Type IB Cas3 and C2 is a Type IE Cas);
(b) C1 is a Type IC or C Cas and C2 is a Type I-E or F Cas (optionally C1 is a Type IC Cas3 and C2 is a Type IE Cas3); or
(c) C1 is a Type II Cas9 and C2 is a Type I Cas3 (optionally C2 is an E coli Type IF or F Cas3; or a C dificile Cas IB).

Optionally,
(a) C1 is a Cas3 (optionally a Type I-A, B, C, D, E, F or U Cas3) and C2 is a Cas3 (optionally a Type I-A, B, C, D, E, F or U Cas3);
(b) C1 is a Cas9 and C2 is a Cas3 (optionally a Type I-A, B, C, D, E, F or U Cas3);
(c) C1 is a Cas3 (optionally a Type I-A, B, C, D, E, F or U Cas3) and C2 is a Cas10 (optionally Cas10 subtype A, B, C or D),
(d) C1 is a Cas9 and C2 is a Cas10 (optionally Cas10 subtype A, B, C or D);
(e) C1 is a Cas9 and C2 is a Cas12 (optionally Cas12a);
(f) C1 is a Cas3 (optionally a Type I-A, B, C, D, E, F or U Cas3) and C2 is a Cas12 (optionally Cas12a);
(g) C1 is a Cas9 and C2 is a Cas13 (optionally Cas13a, Cas13b, Cas13c or Cas13d); or
(h) C1 is a Cas3 (optionally a Type I-A, B, C, D, E, F or U Cas3) and C2 is a Cas13 (optionally Cas13a, Cas13b, Cas13c or Cas13d).

Optionally, C1 is a Clostridiaceae Cas3 (optionally a C dificile Cas3, such as a Type I-B Cas3) and C2 is an Enterobacteriaceae Cas3 (optionally an E coli Cas3, such as a Type I-E Cas3).

In an alternative, C1 and C2 are the same. In an alternative, C1 and C2 are the same type of Cas, eg, each is a Cas9, or each is a Cas3, or each is a Cas12, or each is a Cas13, or each is the same type of Cascade Cas.

Optionally, C1 is a Biostraticola, Buttiauxella, Cedecea, Citrobacter, Cronobacter, Enterobacillus, Enterobacter, Escherichia, Franconibacter, Gibbsiella, Izhakiella, Klebsiella, Kluyvera, Kosakonia, Leclercia, Lellhottia, Limnobaculum, Mangrovibacter, Metakosakonia, Pluralibacter, Pseudescherichia, Pseudocitrobacter, Raoultella or Rosenbergiella Cas (eg, Cas3 or Cascade Cas).

Optionally, C1 is a spCas9 (S pyogenes Cas9) or saCas9 (S aureus Cas9) and C2 is a Type I Cas3 (optionally C2 is an E coli Type I-E or F Cas3).

A suitable protospacer sequence may be a chromosomal sequence of the cell. Alternatively, a suitable protospacer sequence is an episomal (eg, plasmid) sequence of the cell.

Optionally, each cell may be a human, animal (ie, non-human animal), plant, yeast, fungus, amoeba, insect, mammalian, vertebrate, bird, fish, reptile, rodent, mouse, rat, livestock animal, cow, pig, sheep, goat, rabbit, frog, toad, protozoan, invertebrate, molluse, fly, grass, tree, flowering plant, fruiting plant, crop plant, wheat, corn, maize, barley, potato, carrot or lichen cell. Optionally, each cell is a prokaryotic cell or eukaryotic cell. For example, each cell is a bacterial or archaeal cell, optionally an E coli cell or C dificile cell. In an embodiment, the cell or the cells are of a genus or species disclosed in Table 1. In an embodiment, the cell or the cells are gram positive cells. In an embodiment, the cell or the cells are gram negative cells.

C1 may be a Cas3 and the hybrid DNA encodes a Cas5, Cas6, Cas7 and Cas8 (and optionally a Cas11) that are cognate to the Cas3. Additionally or alternatively, optionally C2 may be a Cas3 and the hybrid DNA encodes a Cas5, Cas6, Cas7 and Cas8 (and optionally a Cas11) that are cognate to the Cas3.

The hybrid DNA may encode at least 3, 4 or 5 different types of crRNAs wherein the types target different protospacer sequences comprised by the target cell genome (e,g different chromosomal sequences). In an example, the cell is a bacterial or archaeal cell and the protospacers are comprised by the cell chromosome. For example, at least one or two of said crRNA types targets a respective chromosomal sequence and at least one or more of the crRNA types targets a sequence comprised by an episome (eg, a plasmid) of the cell, wherein the cell is a bacterial or archaeal cell. For example, the cell (eg, a human or mammalian cell) comprises a plurality of chromosomes and the crRNAs target protospacer sequences comprised by two or more of said chromosomes (eg, wherein the chromosomes are not members of the same diploid chromosomal pair).

For example, the hybrid DNA comprises, in 5′ to 3′ direction a nucleotide sequence encoding a Cas nuclease (eg, a cas3) and one or more sequences encoding one or more Cascade Cas (eg, cas8e, cas11, cas7, cas5, and cas6; or cas6, cas8b, cas7, and cas5) that are operable with the Cas nuclease to modify a cognate protospacer sequence.

The hybrid DNA may be devoid of a CRISPR/Cas adaptation module. Optionally, the module encodes a Cas1 and a Cas2; or a Cas1, a Cas2 and a Cas4.

The hybrid may comprise a CRISPR array encoding crRNAs, such as an array comprising at least 3, 4 or 5 spacer sequences targeting at least 3, 4 or 5 sequences of the cell respectively. For example, a plurality of chromosomal intergenic regions are targeted. Optionally, each spacer sequence consists of from 20 to 50, 20 to 40, 22 to 40, 25 to 40 or 30 to 35 consecutive nucleotides, eg, 32 or 37 nucleotides.

In an example, the array comprises the following spacer sequences (Spacers 1-3):

(SEQ ID NO: 130)
TGATTGACGGCTACGGTAAACCGGCAACGTTC;
(SEQ ID NO: 131)
GCTGTTAACGTACGTACCGCGCCGCATCCGGC;
and
(SEQ ID NO: 132)
CGGACTTAGTGCCAAAACATGGCATCGAAATT

separated by repeat sequence (ie, Spacer 1-repeat-Spacer 2-repeat-Spacer 3).

In another example, the array comprises 3, 4 or 5 of the following spacer sequences (Spacers 4-8):

(SEQ ID NO: 133)
GCCATAATCTGGATCAGGAAGTCTTCCTTATCCATAT;
(SEQ ID NO: 134)
GGCTTTACGCCAGCGACGTATTGCCACAGGAATAACT;
(SEQ ID NO: 135)
GGGGATAGCGCGCCTGGAGCGTGCGATAGAGACTTTG;
(SEQ ID NO: 136)
GGCATTTACCGACCAGCCCATCAGCAGTACAGCAAAC;
and
(SEQ ID NO: 137)
TCCTGAATCAAATCCGCCTGTGGCAGGCCATAGCCCG

separated by repeat sequence (ie, Spacer 4-repeat-Spacer 5-repeat-Spacer 6-repeat-Spacer 7-repeat-Spacer 8).

Optionally, each repeat sequence consists of from 20 to 50, 20 to 40, 22 to 40, 25 to 40 or 30 to 35 consecutive nucleotides, eg, 29 nucleotides. For example, each repeat sequence consists of: GAGTTCCCCGCGCCAGCGGGGATAAACCG (SEQ ID NO: 138); (and optionally the Cas is/are E coli Cas). In another example, each repeat sequence consists of (and optionally the Cas is/are C dificile Cas).

Optionally, each crRNA is expressed from the hybrid DNA under the control of a common or respective constitutive promoter.

Optionally, each Cas is expressed from the hybrid DNA under the control of a common or respective constitutive promoter. In an embodiment, the first crRNA and C1 are expressed under the control of a common constitutive promoter and/or the second crRNA and C2 are expressed under the control of a common constitutive promoter. For example, the promoters are the same promoter or they are different promoters. In an example, one, more of all of said promoters is a strong promoter. A promoter may be any promoter disclosed in WO2020078893 or US20200115716, the disclosures of such promoters (and nucleic acids, operons and vectors comprising one or more such promoters) being expressly incorporated herein by reference for possible use in the present invention.

The hybrid DNA may encode (i) a first plurality of different crRNAs for expressed in each cell, wherein each crRNA is operable with a Cas (eg, CS1) to guide modification of the genome and the plurality targets at least 2, 3, 4, 5, 6, 7, 8, 9, 10, 11, 12, 13, 14, 15, 16, 17, 18, 19 or 20 (preferably, at least 2, 3, 4 or 5; or exactly 2, 3, 4 or 5) different protospacers comprised by the genome of the cell; and/or (ii) a second plurality of different crRNAs for expression in each cell wherein each crRNA is operable with a Cas (eg, CS2) and the second plurality targets at least 2, 3, 4, 5, 6, 7, 8, 9, 10, 11, 12, 13, 14, 15, 16, 17, 18, 19 or 20 (preferably, at least 2, 3, 4 or 5; or exactly 2, 3, 4 or 5) different comprised by the genome of the cell. For example, the first plurality comprises from 2 to 10, eg, from 2 to 7, different crRNAs. For example, the second plurality comprises from 2 to 10, eg, from 2 to 7, different crRNAs.

Optionally, the first crRNA (or each crRNA of said first plurality) is comprised by a guide RNA wherein the guide RNA further comprises a tracrRNA and/or the second crRNA (or each crRNA of said second plurality) is comprised by a guide RNA wherein the guide RNA further comprises a tracrRNA. Optionally, the first crRNA (or each crRNA of said first plurality) is comprised by a chimaeric guide RNA and/or the second crRNA (or each crRNA of said second plurality) is comprised by a chimaeric guide RNA.

There is provided:—

A method of killing or reducing the growth or proliferation of a plurality of cells (optionally prokaryotic cells, such as bacterial cells) of a first species or strain, the method comprising infecting the cells with second virus particles disclosed herein, wherein the hybrid DNA of the particles express at least one Cas nuclease (eg. C1 and/or C2) and the genomes of the cells are cut by Cas nuclease cutting and the cells are killed or the growth or proliferation of the cells is reduced.

The method may be carried out ex vivo or in vitro. The method may be carried out in vivo. The method may be carried out in a human or animal subject. The method may be carried out in a fungus, yeast or plant.

Optionally, each cell or the plurality of cells is comprised by a microbiome sample, wherein the method is carried out in vitro and produces a modified cell sample in which cells of the first species or strain have been killed, the method further comprising combining the modified sample with a pharmaceutically acceptable carrier, diluent or excipient, thereby producing a pharmaceutical composition comprising a cell transplant. For example, the transplant may be administered to the gastrointestinal (GI) tract or gut of a human or animal subject, eg, by oral administration, or by rectal administration. For example the transplant may be administered by vaginal administration.

Optionally, a microbiome herein is a gut, lung, kidney, urethral, bladder, blood, vaginal, eye, ear, nose, penile, bowel, liver, heart, tongue, hair or skin microbiome.

The method may reduce the number of cells of said plurality at least 10⁵, 10⁶ or 10⁷ fold, eg, between 10⁵ and 10⁷-fold, or between 10⁵ and 10⁸-fold or between 10⁵ and 10⁹-fold. The skilled person will be familiar with determining fold-killing or reduction in cells, eg, using a cell sample that is representative of a microbiome or cell population. For example, the extent of killing or reduction in growth or proliferation is determined using a cell sample, eg, a sample obtained from a subject to which the composition of the invention has been administered, or an environmental sample (eg, aqueous, water or soil sample) obtained from an environment (eg, a water source, waterway or field) that has been contacted with the composition of the invention. For example, the method reduces the number of cells of said plurality at least 10⁵, 10⁶ or 10⁷-fold and optionally the plurality comprises at least 100,000, 1,000,000; or 10,000,000 cells respectively Optionally, the plurality of cells is comprised by a cell population, wherein at least 5, 6 or 7 log 10 of cells of the population are killed by the method, and optionally the plurality comprises at least 100,000; 1,000,000; or 10,000,000 cells respectively.

Each cell may be a bacterial cell, such as a cell of a first species or genus selected from Table 1. Similarly, a plurality of cells herein may be cells which are of a species or genus selected from Table 1.

Optionally, the method kills at least 99%. 99.9%. 99.99/. 99.99%, 99.9999% or 99.99999% cells of said plurality.

In an example, the method is carried out on a population (or said plurality) of said cells and the method kills, modifies or edits all (or essentially all) of the cells of said population (or said plurality). In an example, the method is carried out on a population (or said plurality) of said cells and the method kills, modifies or edits 100% (or about 100%) of the cells of said population (or plurality).

Optionally, the species is E coli or C difficile.

There is provided:—

A method of editing the genome of one or more cells, the method comprising
(a) modifying the genome of each cell by infecting the cells with second virus particles disclosed herein, wherein the hybrid DNA of the particles express at least one Cas nuclease (eg, C1 and/or C2) and the genomes of the cells are cut by Cas nuclease cutting, wherein the genome is subjected to Cas cutting; and
(b) inserting a nucleic acid at or adjacent to a Cas cut site in the genome and/or deleting a nucleic acid sequence from the genome at or adjacent to a Cas cut site in the genome, wherein a cell with an edited genome is produced; and
(c) optionally isolating from the cell a nucleic acid comprising the insertion or the deletion; or sequencing a nucleic acid sequence of the cell wherein the nucleic acid sequence comprises the insertion or the deletion.

The method may be carried out on a population of said cells, wherein the population comprises at least 100 of said cells and at least 90 or 99% of said cells are edited. The method may be a method of recombineering, eg, in one or more E coli cells.

The insertion may be immediately adjacent to, or overlapping the cut site, or the insertion may be within 1 kb, 2 kb or 200, 150, 100, 50, 25, 10 or 5 nucleotides of the cut site. For example, the nucleic acid is inserted by homologous recombination. In an embodiment, the nucleic acid is inserted by homologous recombination and replaces (the sequence is inserted in the place of genome sequence that is deleted) genome sequence of 1 to 100, 90, 80, 70, 60, 50, 40, 30, 20, 10 or 5 kb, or 200, 150, 100, 50, 25, 10 or 5 nucleotides of the genome. For example, the deleted genome sequence flanks either side of the cut site, or is at the 5′- or 3′-side of the cut site. In an embodiment, the nucleic acid is inserted by homologous recombination and does not replace any genomic sequence.

The deletion may be immediately adjacent to, or overlapping the cut site, or the deletion may be within 1 kb, 2 kb or 200, 150, 100, 50, 25, 10 or 5 nucleotides of the cut site. For example, deletion is a deletion of 1 to 100, 90, 80, 70, 60, 50, 40, 30, 20, 10, 5, 2 or 1 kb, or 200, 150, 100, 50, 25, 10 or 5 nucleotides of the genome. For example, the deleted genome sequence flanks either side of the cut site, or is at the 5′- or 3′-side of the cut site.

For example, the inserted nucleic acid is DNA. For example, the deleted nucleic acid is DNA, eg, chromosomal or episomal DNA).

For example, the inserted nucleic acid is at least (or no more than) 100, 90, 80, 70, 60, 50, 40, 30, 20, 10, 5, 2 or 1 kb; or 200, 150, 100, 50, 25, 10 or 5 consecutive nucleotides in length. For example, the deleted genomic nucleic acid is at least (or no more than) 100, 90, 80, 70, 60, 50, 40, 30, 20, 10, 5, 2 or 1 kb; or 200, 150, 100, 50, 25, 10 or 5 consecutive nucleotides in length.

For example, the genomic sequence is DNA. For example, genomic DNA is deleted or replaced. For example, genomic DNA is deleted or replaced and the editing inserts DNA sequence into the genome (eg, at or flanking the cut site).

For example, the genomic sequence is RNA For example, genomic RNA is deleted or replaced. For example, genomic RNA is deleted or replaced and the editing inserts RNA sequence into the genome (eg, at or flanking the cut site).

Optionally, the method further comprises
(a) culturing the modified cell(s) to produce progeny thereof; and optionally isolating the progeny cells; or
(b) inserting a sequence obtained from a cell in step (c) into a recipient cell and growing a cell line therefrom.

Optionally, the progeny cells or cell line expresses a protein, wherein the protein is encoded (all or in part) by a nucleotide sequence that comprises the inserted nucleic acid sequence, the method further comprising obtaining the expressed protein or isolating the expressed protein from the cells or cell line.

Optionally, the method further comprises combining the progeny cells, cell line or protein with a pharmaceutically acceptable carrier, diluent or excipient, thereby producing a pharmaceutical composition.

The inserted nucleic acid may comprise a transcription and/or translation regulatory element for controlling expression of one or more nucleic acid sequences of the edited genome that are adjacent to the insertion. For example, the inserted nucleic acid comprises a promoter, eg, a constitutive or strong promoter. In another example, the element is a transcription or translation terminator, eg, the inserted sequence comprises a stop codon. In this way, transcription of a gene (or a part of a gene) that is adjacent to the inserted sequence in the edited genome is terminated or prevented or reduced.

In an example, the deleted genomic sequence is a RNA (eg, mRNA) sequence. For example, the deletion of the RNA sequence reduces or prevents expression of an amino acid sequence in the cell, wherein the amino acid sequence is encoded by the deleted RNA sequence. This may be useful for reducing or preventing expression in the cell of a protein comprising the amino acid sequence, such as where the protein is not desirable or required or detrimental to the cell or is a subject or environment that comprises the cell.

There is provided:—

A method of treating or preventing a disease or condition in a human or animal subject, the method comprising (i) administering to the subject a pharmaceutical composition disclosed herein.

Example diseases and conditions are disclosed below.

There is provided:—

An ex vivo or in vitro method of treating an environment or cell sample, the method comprising exposing the environment or sample to a composition of the invention, wherein cells comprised by the environment or sample are modified, edited or killed, or the growth or proliferation of cells of the environment or sample is reduced.

For example, the cells are killed. For example, the cells are edited by the editing method of the invention. Optionally, the treated sample is administered to a human or animal subject or is contacted with an environment.

Optionally, the plurality of cells is comprised by an environmental sample (eg, an aqueous, w ater, oil, petroleum, soil or fluid (such as an air or liquid) sample). A suitable environment may be contents of an industrial or laboratory apparatus or container, eg, a fermentation vessel.

Optionally, the method of the invention is carried out in vitro. Optionally, the method of the invention is carried out ex vivo.

The composition disclosed herein may be an aqueous composition. The composition may be a lyophilised or freeze-dried composition, eg, in a formulation that is suitable for inhaled delivery to a subject.

Optionally, the composition is comprised by a sterile medicament administration device, optionally a syringe, IV bag, intranasal delivery device, inhaler, nebuliser or rectal administration device). Optionally, the composition is comprised by a cosmetic product, dental hygiene product, personal hygiene product, laundry product, oil or petroleum additive, water additive, shampoo, hair conditioner, skin moisturizer, soap, hand detergent, clothes detergent, cleaning agent, environmental remediation agent, cooling agent (eg, an air cooling agent) or air treatment agent.

In an example the composition is comprised by a device for delivering the composition as a liquid or dry powder spray. This may be useful for administration topically to patients or for administration to large environmental areas, such as fields or waterways.

Optionally, the cells are comprise by a gut, lung, kidney, urethral, bladder, blood, vaginal or skin microbiome of the subject.

Optionally, the hybrid DNA encodes at least 2, 3, 4, 5, 6, 7, 8, 9, 10, 11, 12, 13, 14, 15, 16, 17, 18, 19 or 20 (preferably, at least 2, 3, 4 or 5; or exactly 2, 3, 4 or 5, or exactly 8, or at least 8) different types of crRNAs wherein the different types target different protospacer sequences comprised by the cell genome; and optionally each crRNA is operable with a Class 1 Cas nuclease, eg, Cas 3 nuclease.

Optionally, the hybrid DNA encodes a Cas3, Cas8e, Cas11, Cas7, Cas5, and Cas6 (optionally, the Cas are E coli Cas) and/or a nucleic acid encoding a Cas3, Cas6, Cas8b, Cas7, and Cas5 (optionally, the Cas are C dificile Cas). In another example, the hybrid DNA encodes a Cas3, Cas8e, Cas11, Cas7, Cas5, and a nucleic acid encoding a Cas9. In another example, the method comprises introducing into each cell a nucleic acid encoding a Cas3, Cas6, Cas8b, Cas7, and Cas5 and a nucleic acid encoding a Cas9.

The Examples shows the identification of regions that are permissive for deletion and/or insertion of heterologous DNA into phage genomes. In this respect, there is provided the following:—

A synthetic phage, wherein the phage is
(a) a synthetic T4 phage that comprises a deletion of DNA from, and/or an insertion of heterologous DNA into, a region of the genome of the phage corresponding to a region between coordinates
(i) 1887 and 8983;
(ii) 2625 and 8092;
(iii) 1904 and 8113;
(iv) 2668 and 7178;
(v) 7844 and 11117;
(vi) 8643 and 10313;
(vii) 8873 and 12826;
(viii) 9480 and 12224;
(ix) 8454 and 17479; or
(x) 9067 and 16673;
wherein coordinates are with reference to wild-type T4 phage genome (SEQ ID NO: 129); or
(b) a synthetic version of a phage (eg, a T-even phage) that is not a T4 phage, wherein the synthetic phage comprises a deletion of DNA from a region of its genome that is homologous or orthologous to said region of (a);
and
wherein the synthetic phage is capable of replication in a host bacterial cell.

Optionally, the deletion comprises up to 8000 bp of DNA.

A method of producing a synthetic phage, the method comprising
(a) providing a heterologous DNA comprising an insert;
(b) providing a first phage genomic DNA;
(c) allowing homologous recombination between a first region of the genomic DNA and the heterologous DNA and allowing homologous recombination between a second region of the genomic DNA and the heterologous DNA,
wherein the insert is inserted between said regions whereby a hybrid DNA is produced that encodes the genome of a synthetic phage; and
wherein
A:
(i) the coordinates of the first region are 1887-2625 and the coordinates of the second region are 8092-8983;
(ii) the coordinates of the first region are 1904-2668 and the coordinates of the second region are 7178-8113;
(iii) the coordinates of the first region are 7844-8643 and the coordinates of the second region are 10313-11117;
(iv) the coordinates of the first region are 8873-9480 and the coordinates of the second region are 12224-12826; or
(v) the coordinates of the first region are 8454-9067 and the coordinates of the second region are 16673-17479;
wherein the first phage is a T4 phage and the coordinates are with reference to wild-type T4 phage genome (SEQ ID NO: 129);
or
B: the first phage (eg, a T-even phage) is not a T4 phage, and wherein the first and second regions are regions of the first phage genome that are homologous or orthologous to said first and second regions of any one of A(i) to (v).

Preferably, the synthetic phage is capable of replication in a host bacterial cell.

A synthetic phage obtainable by the method of the immediately preceding paragraph; or a composition comprising a plurality of synthetic phages, wherein each phage is obtainable by the method of the immediately preceding paragraph.

The DNA insertion may encode one or more components of a CRISPR/Cas system; optionally wherein the DNA insertion encodes one or more different crRNAs or guide RNAs and/or encodes one or more Cas.

The insertion can be an insertion of Xbp of DNA as described herein. The insertion can be a heterologous DNA insertion as described herein.

The insertion may comprise a total number (X) of base pairs of heterologous DNA, and (a) the deletion comprises a total number (Y) of base pairs of DNA wherein Y is at least 50% of X; or (b) the T4 phage or said phage that is not a T4 comprises a capsid that has a DNA packaging capacity of Zbp and the total number of base pairs of the genomic DNA of the synthetic phage is 90-110% of Z.

The insertion may comprise up to 8000 bp of DNA.

The synthetic phage may be a lytic phage; and/or said phage that is not a T4 phage may be lytic phage.

There is provided: A DNA comprising the genome of the synthetic phage: optionally wherein the DNA is a chromosome of a bacterial cell or an episome (eg, a plasmid) comprised by a bacterial cell, such as a host cell of said synthetic phage.

The DNA insertion may comprise or encode
A. one or more components of a CRISPR/Cas system or a guided nuclease (eg, a Cas, TALEN, meganuclease or zinc finger); optionally wherein the heterologous DNA encodes a guide RNA (eg, a single guide RNA) and/or a Cas (eg, a Cas9, Cas3, Cas12, Cas13 or Cas14);
B. an antibacterial agent;
C. a phage tail fibre or component thereof;
D. a vitamin;
E. a blood protein;
F. an antibody or fragment thereof; or
G. a human or plant protein or fragment thereof.

Regarding the synthetic phage, method, composition or DNA, said phage that is not a T4 phage may be selected from the group consisting of the phages of Table 6, Escherichia phage T4, Escherichia phage T2, Escherichia phage T6,m Escherichia phage RB69, Shigella phage Shf125875, Escherichia phage APCEc01, Escherichia phage moskry, Escherichia phage ST0, Escherichia phage vB_EcoM_JS09, Shigella phage SP18, Escherichia phage vB_EcoM_PhAPEC2, Escherichia phage HX1, Salmonella phage SG1, Shigella phage pSs-1, Escherichia phage HY01, Yersinia phage PST, Escherichia phage AR1, Escherichia phage phiE142, Shigella phage SHFML-11, Escherichia phage slur07, Shigella phage SHFML-11, Escherichia phage UFV-AREG1, Escherichia phage vB_EcoM-UFV13, Shigella phage JK38, Shigella phage SHFML-26, Shigella phage Sf22, Escherichia phage ime09, Shigella phage SH7, Yersinia phage phiD1, Escherichia phage RB3, Escherichia phage ECML-134, Escherichia phage vB_EcoM_ACG-C40, Escherichia phage vB_EcoM-fFiEco06, Escherichia phage PP01, Shigella phage Shfl2, Escherichia phage ECO4, Escherichia virus RB14, Escherichia phage vB_EcoM_JB75, Shigella phage Sf22, Escherichia phage vB_vPM_PD112, Shigella phage Sf23, Escherichia phage vB_EcoM_G2540, Escherichia phage vB_EcoM_G2133, Escherichia phage vB_EcoM_G4498, Escherichia virus RB32, Escherichia phage vB_EcoM_G4507, Escherichia phage vB_EcoM_G8, Escherichia phage EcNP 1, Enterobacteria phage RB27, Shigella virus KRT47, Escherichia phage teqdroes, Escherichia phage slur02, Yersinia phage fPS-90, Yersinia phage phiD1, Shigella phage Sf24 and Escherichia phage phiC120.

There is provided:

A method of producing synthetic phage particles, comprising
(i) Allowing the production of synthetic phage in producer cells, wherein the phage are according to the invention; and
(ii) Isolating the phage; and
(iii) Optionally combining a population of said isolated synthetic phage with a pharmaceutically acceptable excipient, carrier or diluent to produce a pharmaceutical composition.

A method of producing a pharmaceutical composition, the method comprising combining a population of synthetic phage with a pharmaceutically acceptable excipient, carrier or diluent to produce a pharmaceutical composition, wherein the phage are according to the invention.

A population of synthetic phage according to the invention; or a pharmaceutical composition obtainable by the method of the invention, for use as a medicament; optionally for administration to a human or animal subject for reducing infection by pathogenic host bacterial or archaeal cells or a first species or strain, wherein the phage are capable of infecting cells of said species or strain.

### Diseases and Conditions

Optionally, the disease or condition is selected from
(a) A neurodegenerative disease or condition;
(b) A brain disease or condition;
(c) A CNS disease or condition;
(d) Memory loss or impairment;
(e) A heart or cardiovascular disease or condition, eg, heart attack, stroke or atrial fibrillation;
(f) A liver disease or condition;
(g) A kidney disease or condition, eg, chronic kidney disease (CKD);
(h) A pancreas disease or condition;
(i) A lung disease or condition, eg, cystic fibrosis or COPD;
(j) A gastrointestinal disease or condition;
(k) A throat or oral cavity disease or condition;
(l) An ocular disease or condition;
(m) A genital disease or condition, eg, a vaginal, labial, penile or scrotal disease or condition;
(n) A sexually-transmissible disease or condition, eg, gonorrhea, HIV infection, syphilis or Chlamydia infection;
(o) An ear disease or condition;
(p) A skin disease or condition;
(q) A heart disease or condition;
(r) A nasal disease or condition
(s) A haematological disease or condition, eg, anaemia, eg, anaemia of chronic disease or cancer,
(t) A viral infection;
(u) A pathogenic bacterial infection;
(v) A cancer;
(w) An autoimmune disease or condition, eg, SLE;
(x) An inflammatory disease or condition, eg, rheumatoid arthritis, psoriasis, eczema, asthma, ulcerative colitis, colitis, Crohn's disease or IBD;
(y) Autism;
(z) ADHD;
(aa) Bipolar disorder;
(bb) ALS [Amyotrophic Lateral Sclerosis];
(cc) Osteoarthritis;
(dd) A congenital or development defect or condition;
(ee) Miscarriage;
(ff) A blood clotting condition;
(gg) Bronchitis;
(hh) Dry or wet AMD;
(ii) Neovasculansation (eg, of a tumour or in the eye);
(jj) Common cold;
(kk) Epilepsy;
(ii) Fibrosis, eg. liver or lung fibrosis;
(mm) A fungal disease or condition, eg, thrush;
(nn) A metabolic disease or condition, eg, obesity, anorexia, diabetes, Type I or Type II diabetes.
(oo) Ulcer(s), eg, gastric ulceration or skin ulceration;
(pp) Dry skin;
(qq) Sjogren's syndrome;
(rr) Cytokine storm;
(ss) Deafness, hearing loss or impairment;
(tt) Slow or fast metabolism (ie, slower or faster than average for the weight, sex and age of the subject);
(uu) Conception disorder, eg, infertility or low fertility;
(vv) Jaundice;
(ww) Skin rash;
(xx) Kawasaki Disease;
(yy) Lyme Disease;
(zz) An allergy, eg, a nut, grass, pollen, dust mite, cat or dog fur or dander allergy;
(aaa) Malaria, typhoid fever, tuberculosis or cholera;
(bbb) Depression;
(ccc) Mental retardation;
(ddd) Microcephaly;
(eee) Malnutrition;
(fff) Conjunctivitis;
(ggg) Pneumonia;
(hhh) Pulmonary embolism;
(iii) Pulmonary hypertension;
(jjj) A bone disorder;
(kkk) Sepsis or septic shock;
(lll) Sinusitus;
(mmm) Stress (eg, occupational stress);
(nnn) Thalassaemia, anaemia, von Willebrand Disease, or haemophilia;
(ooo) Shingles or cold sore;
(ppp) Menstruation;
(qqq) Low sperm count.

### Neurodegenerative or CNS Diseases or Conditions for Treatment or Prevention

In an example, a neurodegenerative or CNS disease or condition is selected from the group consisting of Alzheimer disease, geriopsychosis, Down syndrome, Parkinson's disease, Creutzfeldt-jakob disease, diabetic neuropathy, Parkinson syndrome, Huntington's disease, Machado-Joseph disease, amyotrophic lateral sclerosis, diabetic neuropathy, and Creutzfeldt Creutzfeldt-Jakob disease. For example, the disease is Alzheimer disease. For example, the disease is Parkinson syndrome.

In an example, wherein the method of the invention is practised on a human or animal subject for treating a CNS or neurodegenerative disease or condition, the method causes downregulation of Treg cells in the subject, thereby promoting entry of systemic monocyte-derived macrophages and/or Treg cells across the choroid plexus into the brain of the subject, whereby the disease or condition (eg, Alzheimer's disease) is treated, prevented or progression thereof is reduced. In an embodiment the method causes an increase of IFN-gamma in the CNS system (eg, in the brain and/or CSF) of the subject. In an example, the method restores nerve fibre and/or reduces the progression of nerve fibre damage. In an example, the method restores nerve myelin and/or reduces the progression of nerve myelin damage. In an example, the method of the invention treats or prevents a disease or condition disclosed in WO2015136541 and/or the method can be used with any method disclosed in WO2015136541 (the disclosure of this document is incorporated by reference herein in its entirety, eg, for providing disclosure of such methods, diseases, conditions and potential therapeutic agents that can be adminstered to the subject for effecting treatment and/or prevention of CNS and neurodegenerative diseases and conditions, eg, agents such as immune checkpoint inhibitors, eg, anti-PD-1, anti-PD-L1, anti-TIM3 or other antibodies disclosed therein).

### Cancers for Treatment or Prevention

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Haematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas) Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medu!loblastoma, Schwannoma craniopharyogioma, ependymoma, pineaioma, hemangioblastoma, acoustic neuroma, oligodendroglioma, menangioma, neuroblastoma, retinoblastoma and brain metastases).

### Autoimmune Diseases for Treatment or Prevention

Acute Disseminated Encephalomyelitis (ADEM)
Acute necrotizing hemorrhagic leukoencephahtis
Addison's disease
Agammaglobulnemia
Alopecia areata
Amyloidosis
Ankylosing spondylitis
Anti-GBM/Anti-TBM nephritis
Antiphospholipid syndrome (APS)
Autoimmune angioedema
Autoimmune aplastic anemia
Autoimmune dysautonomia
Autoimmune hepatitis
Autoimmune hyperlipidemia
Autoimmune immunodeficiency
Autoimmune inner ear disease (AIED)
Autoimmune myocarditis
Autoimmune oophoritis
Autoimmune pancreatitis
Autoimmune retinopathy
Autoimmune thrombocytopenic purpura (ATP)
Autoimmune thyroid disease
Autoimmune urticaria
Axonal & neuronal neuropathies
Balo disease
Behcet's disease
Bullous pemphigoid
Cardiomyopathy
Castleman disease
Celiac disease
Chagas disease
Chronic fatigue syndrome
Chronic inflammatory demyelinating polyneuropathy (CIDP)
Chronic recurrent multifocal ostomyelitis (CRMO)
Churg-Strauss syndrome
Cicatricial pemphigoid/benign mucosal pemphigoid
Crohn's disease
Cogans syndrome
Cold agglutinin disease
Congenital heart block
Coxsackie myocarditis
CREST disease
Essential mixed cryoglobulinemia
Demyelinating neuropathies
Dermatitis herpetiformis
Dermatomyositis
Devic's disease (neuromyelitis optica)
Discoid lupus
Dressler's syndrome
Endometriosis
Eosinophilic esophagitis
Eosinophilic fasciitis
Erythema nodosum
Experimental allergic encephalomyelitis
Evans syndrome
Fibromyalgia
Fibrosing alveolitis
Giant cell arteritis (temporal arteritis)
Giant cell myocarditis
Glomerulonephritis
Goodpasture's syndrome
Granulomatosis with Polyangiitis (GPA) (formerly called Wegener's Granulomatosis)
Graves' disease
Guillain-Barre syndrome
Hashimoto's encephalitis
Hashimoto's thyroiditis
Hemolytic anemia
Henoch-Schonlein purpura
Herpes gestationis
Hypoganmaglobulinemia
Idiopathic thrombocytopenic purpura (ITP)
IgA nephropathy
IgG4-related sclerosing disease
Immunoregulatory lipoproteins
Inclusion body myositis
Interstitial cystitis
Juvenile arthritis
Juvenile diabetes (Type I diabetes)
Juvenile myositis
Kawasaki syndrome
Lambert-Eaton syndrome
Leukocytoclastic vasculitis
Lichen planus
Lichen sclerosus
Ligneous conjunctivitis
Linear IgA disease (LAD)
Lupus (SLE)
Lyme disease, chronic
Meniere's disease
Microscopic polyangiitis
Mixed connective tissue disease (MCTD)
Mooren's ulcer
Mucha-Habermann disease
Multiple sclerosis
Myasthenia gravis
Myositis
Narcolepsy
Neuromyelitis optica (Devic's)
Neutropenia
Ocular cicatricial pemphigoid
Optic neuritis
Palindromic rheumatism
PANDAS (Pediatric Autoimmune Neuropsychiatric Disorders Associated with Streptococcus)
Paraneoplastic cerebellar degeneration
Paroxysmal nocturnal hemoglobinuria (PNH)
Parry Romberg syndrome
Parsonnage-Turner syndrome
Pars planitis (peripheral uveitis)
Pemphigus
Peripheral neuropathy
Perivenous encephalomyelitis
Pernicious anemia
POEMS syndrome
Polyarteritis nodosa
Type I, II, & III autoimmune polyglandular syndromes
Polymyalgia rheumatica
Polymyositis
Postmyocardial infarction syndrome
Postpericardiotomy syndrome
Progesterone dermatitis
Primary biliary cirrhosis
Primary sclerosing cholangitis
Psoriasis
Psoriatic arthritis
Idiopathic pulmonary fibrosis
Pyoderma gangrenosum
Pure red cell aplasia
Raynauds phenomenon
Reactive Arthritis
Reflex sympathetic dystrophy
Reiter's syndrome
Relapsing polychondritis
Restless legs syndrome
Retroperitoneal fibrosis
Rheumatic fever
Rheumatoid arthritis
Sarcoidosis
Schmidt syndrome
Scleritis
Scleroderma
Sjogren's syndrome
Sperm & testicular autoimmunity
Stiff person syndrome
Subacute bacterial endocarditis (SBE)
Susac's syndrome
Sympathetic ophthalmia
Takayasu's arteritis
Temporal arteritis/Giant cell arteritis
Thrombocytopenic purpura (TTP)
Tolosa-Hunt syndrome
Transverse myelitis
Type I diabetes
Ulcerative colitis
Undifferentiated connective tissue disease (UCTD)
Uveitis
Vasculitis
Vesiculobullous dermatosis
Vitiligo
Wegener's granulomatosis (now termed Granulomatosis with Polyangiitis (OPA).

### Inflammatory Diseases for Treatment or Prevention

Alzheimer's
ankylosing spondylitis
arthritis (osteoarthitis, rheumatoid arthritis (RA), psoriatic arthritis)
asthma
atherosclerosis
Crohn's disease
colitis
dermatitis
diverticulitis
fibromyalgia
hepatitis
irritable bowel syndrome (IBS)
systemic lupus erythematous (SLE)
nephritis
Parkinson's disease
ulcerative colitis.

Optionally, the cell(s) are C dificile, P aeruginosa, K pneumoniae (eg, carbapenem-resistant Klebsiella pneumoniae or Extended-Spectrum Beta-Lactamase (ESBL)-producing K pneumoniae), E coli (eg, ESBL-producing E. coli, or E. coli ST131-O25b:H4), H pylori, S pneumoniae or S aureus cells.

The hybrid DNA may comprise a promoter for expression of one or more products encoded by the heterologous DNA (eg, for expression of one or more crRNAs) In an example, promoter is a medium strength promoter. In another example, the promoter is a repressible promoter or an inducible promoter cell. Examples of suitable repressible promoters are Ptac (repressed by lacI) and the Leftward promoter (pL) of phage lambda (which repressed by the λcI repressor). In an example, the promoter comprises a repressible operator (eg, tetO or lacO) fused to a promoter sequence. Optionally, the promoter has an Anderson Score (AS) of 0.5>AS>0.1.

### Paragraphs:

By way of illustration of the various aspects of the disclosure, there are provided the following Paragraphs (which are not to be construed as claims, the claims follow below starting with the title “CLAIMS”).
1. A method of producing a modified genome of a first virus, wherein the modified genome comprises a total number (X) of base pairs of heterologous DNA, wherein the first virus is capable of infecting a target cell of a first species or strain, the method comprising
   (a) obtaining sequence(s) of the genome of the first virus at least to the extent comprising a first set of genes required for virus particle production in a host cell; and
   (b) producing a hybrid DNA comprising the sequence(s) obtained in step (a) and said heterologous DNA, wherein the hybrid DNA comprises said modified genome;
   Wherein
   (c) the modified genome is functional to produce a second virus that is capable of infecting the target cell, the second virus comprising proteins encoded by said set of genes, wherein the proteins package hybrid DNA comprising said heterologous DNA and said set of genes, wherein the second virus is a modified version of the first virus; and
   (d) A: the hybrid DNA excludes a total number (Y) of base pairs of DNA of the genome of the first virus wherein Y is at least 49 or 50% of X; or
      B: the second virus comprises a capsid that has a DNA packaging capacity of Zbp and the total number of base pairs of the hybrid DNA is 90-110% of Z.
2. The method of paragraph 1, wherein the method comprises
   (i) obtaining DNA from a said first virus, wherein the DNA comprises said set of genes;
   (ii) sequencing the DNA of step (i);
   (iii) comparing the sequence of the DNA obtained in step (ii) with a reference viral genome sequence, by
      I. aligning the DNA sequence obtained in step (ii) with the reference sequence;
      II. identifying a reference set of genes comprised by the reference sequence wherein the genes are genes required for reference virus particle production and replication;
      III. identifying in the aligned DNA sequence said first set of genes wherein the first set of genes corresponds to the reference set of genes; and
   (iv) producing said hybrid DNA comprising said first set of genes identified in step III and said heterologous DNA.
3. The method of paragraph 2, wherein step III comprises
   IV. identifying open reading frame (ORF) sequences in the aligned sequence (First Set ORFs) and comparing the First Set ORFs with ORFs in the reference sequence, wherein ORFs of the aligned sequence that correspond to ORFs of the reference sequence that are comprised by said reference set of genes are identified, whereby genes of the first set are identified as genes comprising the First Set ORFs.
4. The method of paragraph 3, wherein step IV comprises
   V. BLAST analysis of the sequence obtained in step (ii) with viral genome sequences comprised by a database of viral genome sequences, optionally a Genbank database.
5. The method of paragraph 2 or 3, wherein step (iv) comprises
   VI. deleting at least Xbp of DNA from a DNA comprising the first virus genome to produce a second DNA, wherein the deletion does not include nucleotides of the first set of genes or does not render the first set of genes non-functional for virus replication and production; and inserting the heterologous DNA into the second DNA to produce the hybrid DNA;
   VII. inserting the heterologous DNA into a DNA comprising the first virus genome to produce a second DNA; and deleting from the second DNA at least Xbp of DNA to produce the hybrid DNA, wherein the deletion does not include nucleotides of the first set of genes or does not render the first set of genes non-functional for virus replication and production; or
   VIII. carrying out said deletion and insertion simultaneously on a DNA comprising the first virus genome, thereby producing the hybrid DNA.
6. The method of any preceding paragraph, wherein (i) Xbp is 2-15 kbp (eg. 7-9 kbp) and/or Ybp is 1-20 kbp (eg, 3-9 kbp); or (ii) Y is 50-200% (optionally, 50-100%) of X; or (iii) Zbp is 4 to 600 kbp.
7. The method of any preceding paragraph, wherein the heterologous DNA encodes a first tail fibre or component thereof and/or the excluded DNA encodes a second tail fibre or component thereof, wherein the first and second tail fibres or components are different from each other.
8. The method of any preceding paragraph, wherein the heterologous DNA encodes a guided nuclease (optionally a Cas) and/or a guide RNA and/or the heterologous DNA comprises a CRISPR array for producing a crRNA in the target cell.
9. The method of any preceding paragraph, wherein the heterologous DNA encodes a virus tail fibre and a guide RNA; or the heterologous DNA encodes a virus tail fibre and comprises a CRISPR array for producing a crRNA in the target cell.
10. The method of any preceding paragraph, wherein each virus is a phage (eg, an enterobacteria phage, E coli phage or Caudovirales phage), adeno-associated virus (AAV), herpes simplex virus, retrovirus or lentivirus.
11. The method of any preceding paragraph, wherein each virus is a T-even phage.
12. The method of paragraph 11, wherein each virus is a phage selected from the group consisting of Escherichia phage T4, Escherichia phage T2, Escherichia phage T6,m Escherichia phage RB69, Shigella phage Shfl25875, Escherichia phage APCEc01, Escherichia phage moskry, Escherichia phage ST0, Escherichia phage vB_EcoM_JS09, Shigella phage SP18, Escherichia phage vB3_EcoM_PhAPEC2, Escherichia phage HX01, Salmonella phage SG1, Shigella phage pSs-1, Escherichia phage HY01, Yersinia phage PST, Escherichia phage AR1, Escherichia phage phiE142, Shigella phage SHFML-11, Escherichia phage slur07, Shigella phage SHFML-11, Escherichia phage UFV-AREG1, Escherichia phage vB_EcoM-UFV13, Shigella phage JK38, Shigella phage SHFML-26, Shigella phage Sf22, Escherichia phage ime09, Shigella phage SH7, Yersinia phage phiD1, Escherichia phage RB3, Escherichia phage ECML-134, Escherichia phage vB_EcoM_ACG-C40, Escherichia phage vB_EcoM-fFiEco06, Escherichia phage PP01, Shigella phage Shfl2, Escherichia phage ECO4, Escherichia virus RB14, Escherichia phage vB_EcoM_JB75, Shigella phage Sf22, Escherichia phage vB_vPM_PD112, Shigella phage Sf23, Escherichia phage vB_EcoM_G2540, Escherichia phage vB_EcoM_G2133, Escherichia phage vB_EcoM_G4498, Escherichia virus RB32, Escherichia phage vB_EcoM_G4507, Escherichia phage vB_EcoM_G8, Escherichia phage EcNP 1, Enterobacteria phage RB27, Shigella virus KRT47, Escherichia phage teqdroes, Escherichia phage slur02, Yersinia phage fPS-90, Yersinia phage phiD1, Shigella phage Sf24 and Escherichia phage phiC120.
13. The method of paragraph 11, wherein each virus is a T4 phage.
14. The method of any preceding paragraph, wherein each virus is a phage and the hybrid DNA excludes a DNA sequence that is comprised by a gene of the first virus genome, wherein the gene encodes an amino acid sequence selected from SEQ ID Nos: 1-128, or a homologue thereof, optionally wherein the homologue is an amino acid sequence that is at least 80% identical to said selected sequence.
15. The method of paragraph 14, wherein the hybrid DNA excludes a plurality of DNA sequences of the first virus genome, wherein each DNA sequence is comprised by a respective gene of the first virus genome, wherein the gene encodes an amino acid sequence selected from SEQ ID Nos: 1-128, or a homologue thereof-; optionally wherein the homologue is an amino acid sequence that is at least 80% identical to said selected sequence.
16. The method of paragraph 14, wherein each virus is a T even (eg, a T4) phage and the hybrid DNA excludes one or more DNA sequences of the first virus genome, wherein each DNA sequence is comprised by a respective gene of the first virus genome, wherein the gene encodes an amino acid sequence selected from SEQ ID Nos: 1-42, or a homologue thereof; optionally wherein the homologue is an amino acid sequence that is at least 80% identical to said selected sequence.
17 The method of paragraph 14, wherein each virus is a phage (such as a T even phage) and the hybrid DNA excludes one or more DNA sequences of the first virus genome, wherein each DNA sequence is comprised by at least 10% of a respective gene of the first virus genome, wherein (i) the gene encodes an amino acid sequence selected from SEQ ID Nos: 1-42, or a homologue thereof; optionally wherein the homologue is an amino acid sequence that is at least 80% identical to said selected sequence; or (ii) the gene is selected from T4 phage genes 49.1, 49.2, 49.3, nrdC, nrdC.1, nrdC.2, nrdC.3, nrdC.4, nrdC.5, nrdC.6, nrdC.7, nrdC.8, nrdC.9, nrdC.10, nrdC.11, mobD, mobD.1, mobD.2, mobD.2a, mobD.3, mobD.4, mobD.5, rI-1, rl, rl.1, tk, tk.1, tk.2, tk.3, tk.4, vs, vs.1, regB, vs.3, vs.4, vs.5, vs.6, vs.7, vs.8, denV, ipIII and ipII; or an orthologue or homologue thereof.
18. The method of paragraph 14, wherein the hybrid DNA excludes one or more genes of the first virus genome, wherein each gene is selected from T4 phage genes 49.1, 49.2, 49.3, nrdC, nrdC.1, nrdC.2, nrdC 3, nrdC.4, nrdC.5, nrdC.6, nrdC 7, nrdC.8, nrdC.9, nrdC.10, nrdC.11, mobD, mobD.1, mobD.2, mobD.2a, mobD.3, mobD.4, mobD.5, rI.-1, rI, rI.1, tk, tk.1, tk.2, tk.3, tk.4, vs, vs.1, regB, vs.3, vs.4, vs.5, vs.6, vs.7, vs.8, denV, IpIII and IpII; or an orthologue or homologue thereof.
19 The method of any preceding paragraph, wherein the hybrid DNA excludes DNA from 2 or more genes of the first virus genome.
20. The method of any one of paragraphs 14 to 19, wherein each gene encodes a protein selected from a thioredoxin, endonuclease (optionally a homing endonuclease, a RegB site-specific RNA endonuclease or a site-specific intron-like DNA endonuclease), lysis inhibition regulator, membrane protein, thymidine kinase, protein that contains a A1pp phosphatase motif, tRNA synthetase modifier (optionally a valyl-tRNA synthetase modifier), mRNA processing protein, UV repair enzyme (optionally a N-glycosylase UV repair enzyme), internal head protein (eg, a IpIII internal head protein or a Ipil internal head protein, Ip4 protein), endoribonuclease and DNA glycosylase (optionally a pyrimidine dimer DNA glycosylase).
21. The method of any preceding paragraph, wherein the second virus comprises a capsid that has a DNA packaging capacity that is from 90-110% of the packaging capacity of the first virus.
22. The method of any preceding paragraph, wherein the hybrid DNA is 90-110% the size of the DNA of the first virus genome.
23. The method of any preceding paragraph, wherein the second virus comprises a capsid that has a DNA packaging capacity of Zbp and the total number of base pairs of the hybrid DNA is 90-110% of Z.
24. The method of any preceding paragraph, wherein the size of the first virus genome is 90-100% of Z; and/or the size of the first virus genome is smaller than Z by 5-50% of X.
25. The method of any preceding paragraph, wherein the first and second viruses have the same DNA packaging capacity.
26. The method of any preceding paragraph, wherein each virus comprises a life cycle having a lytic pathway, optionally wherein (i) each virus is a lytic virus; or (ii) the first virus is a temperate virus having a life cycle comprising a lytic pathway and a lysogenic pathway, wherein the second virus has a life cycle comprising a lytic pathway but no lysogenic pathway or a disrupted lysogenic pathway wherein the second virus has a reduced chance of entering a lysogenic pathway than the first virus.
27. A method of producing synthetic virus particles, comprising carrying out the method of any preceding paragraph to produce the hybrid DNA, introducing the hybrid DNA into a target cell of a first species or strain in which the hybrid DNA is capable of being replicated and particles of said second virus are produced; and producing second viruses in the cell; and further optionally isolating second virus particles from the cell.
28. The method of paragraph 27, further producing a pharmaceutical composition comprising second virus particles obtained by the method of paragraph 27 and a pharmaceutically acceptable excipient, carrier or diluent.
29. A method of selecting a synthetic virus, the method comprising
   (a) Providing a first type (T1) of a virus, wherein the virus is obtained or obtainable by the method of paragraph 27;
   (b) Providing a second type (T2) of a virus, wherein the virus is obtained or obtainable by the method of paragraph 27, wherein T1 and T2 differ from each other by at least said heterologous DNA comprised by each type (eg, T1 and T2 differ by heterologous DNA encoding first and second tail fibres respectively, wherein the tail fibres are different);
   (c) Culturing the T1 virus with target cells of the first species or strain; and culturing the T2 virus with target cells of the first species or strain;
   (d) Determining which of the cultured T1 and T2 viruses produces a predetermined indicator or the extent of production of the indicator by the viruses;
   (e) Selecting T1 or T2 virus on the basis of the determination in step (d); and
   (f) Optionally further producing further copies of the selected virus and/or determining the sequence of the heterologous DNA or a portion thereof comprised by the selected virus.
30. The method of paragraph 29, wherein T1 viruses are capable of infecting target cells in step (c), but T2 viruses are not capable of infecting of target cells in step (c) or are less infective than T1 viruses; wherein step (d) comprises determining the extent of target cell infectivity of each of T1 and T2, optionally by determining the titres of T1 and T2 viruses that have been cultured.
31. The method of paragraph 29 or 30, wherein the method is carried out using at least 5 different types of second virus, wherein the types differ from each other by their said heterologous DNAs (optionally wherein the types comprise DNA encoding different tail fibres).
32. A virus infectivity assay, the assay comprising
   (a) providing a first type (T1) of virus comprising a first DNA sequence;
   (b) providing a second type (T2) of virus comprising a second DNA sequence, wherein T1 and T2 differ from each other by said DNA sequences and differ in infectivity of target cells;
   (c) Culturing the T1 virus with target cells of a first species or strain; and culturing the T2 virus with target cells of the first species or strain;
   (d) Determining which of the cultured T1 and T2 viruses produces a predetermined indicator or the extent of production of the indicator by the viruses;
   (e) Selecting T1 or T2 virus on the basis of the determination in step (d); and
   (f) Optionally further producing further copies of the selected virus and/or determining the sequence of said DNA or a portion thereof comprised by the selected virus.
33. The assay of paragraph 32, wherein the T1 virus is capable of infecting target cells in step (c), but T2 virus is not capable of infecting of target cells in step (c) or is less infective than T1 virus; wherein step (d) comprises determining the extent of target cell infectivity of each of T1 and T2, optionally by determining the titres of T1 and T2 viruses that have been cultured.
34. The assay of paragraph 32, wherein T1 and T2 differ only by DNA sequences encoding first and second tail fibres respectively, wherein the tail fibres are different; or wherein the T1 and T2 viruses differ only by their tail fibres.
35. The assay of paragraph 33 or 34, wherein the assay is carried out using at least 5 different types of virus (optionally wherein the types comprise DNA encoding different tail fibres).
36. A method of producing a composition comprising synthetic virus particles, the method comprising obtaining particles of a first type from a culture and optionally combining the obtained particles with an excipient, carrier or diluent, wherein the culture comprises target cells, each cell comprising DNA comprising the genome of the first type of virus, wherein the virus comprises the sequence determined in step (f) of paragraph 29 (or paragraph 30 or 31 when dependent from paragraph 29) or step (f) of paragraph 32 (or paragraph 33, 34 or 35 when dependent from paragraph 32).
37. A method of producing synthetic virus particles, the method comprising
   (a) culturing target cells, each cell comprising DNA comprising the genome of a first type of virus, wherein the virus comprises the sequence determined in step (f) of paragraph 29 (or paragraph 30 or 31 when dependent from paragraph 29);
   (b) producing virus particles in the cells;
   (c) obtaining virus particles from the cell culture and
   (d) optionally combining the obtained particles with a pharmaceutically acceptable excipient, carrier or diluent.
38. The method of paragraph 36 or 37, wherein each virus is as recited in any of paragraphs 10-18.
39. A synthetic phage, wherein the phage is
   (a) a synthetic T4 phage comprising a deletion of DNA from a Deletion Permissive Region (DPR) of the genome of the phage, wherein the region is between the pin (protease inhibitor) gene and the iPII (internal protein) gene; or
   (b) a synthetic version of a phage (eg, a T-even phage) that is not a T4 phage, wherein the synthetic phage comprises a deletion of DNA from a region of its genome that is homologous or orthologous to said DPR of (i); and
wherein the synthetic phage is capable of replication in a host cell.
40. The synthetic phage of paragraph 39, wherein the deletion comprises up to 8000 bp of DNA.
41. The synthetic phage of paragraph 39 or 40, wherein the synthetic phage of (i) comprises an insertion of heterologous DNA, wherein the insertion is between the pin gene and the ipII gene, or the synthetic phage of (ii) comprises an insertion of heterologous DNA, wherein the insertion is between a first gene and a second gene, wherein the first gene is homologous or orthologous to the pin gene of T4 and the second gene is homologous or orthologous to the ipII gene of T4.
42. The synthetic phage of paragraph 41, wherein the insertion comprises a total number (X) of base pairs of heterologous DNA, and (a) the deletion comprises a total number (Y) of base pairs of DNA wherein Y is at least 50% of X; or (b) the T4 phage or said phage that is not a T4 comprises a capsid that has a DNA packaging capacity of Zbp and the total number of base pairs of the genomic DNA of the synthetic phage is 90-110% of Z.
43. A synthetic phage, wherein the phage is
   (a) a synthetic T4 phage comprising an insertion of heterologous DNA into a Deletion Permissive Region (DPR) of the genome of the phage, wherein the region is between the pin (protease inhibitor) gene and the ipII (internal protein) gene; or
   (b) a synthetic version of a phage that is not a T4 phage, wherein the synthetic phage comprises an insertion of heterologous DNA into a region of its genome that is homologous or orthologous to said DPR of (i); and
wherein the synthetic phage is capable of replication in a host cell.
44. The synthetic phage of any one of paragraphs 41 to 43, wherein the insertion comprises up to 8000 bp of DNA.
45. The synthetic phage of any one of paragraphs 39 to 44, wherein the DPR of the T4 phage comprises contiguous DNA between the pin gene and the ipII gene, wherein the contiguous DNA is at least 1000 bp in length; or wherein the DPR of the 14 phage comprises at least 100 bp of DNA between the pin gene and the ipII gene.
46. The synthetic phage of any one of paragraphs 39 to 45, wherein the DPR of the T4 phage extends from the pin gene to the ipII gene.
47. The synthetic phage of any one of paragraphs 39 to 46, wherein
   A. the synthetic phage genome comprises a deletion of a one or more genes, wherein each gene encodes a protein selected from a thioredoxin, endonuclease (optionally a homing endonuclease, a RegB site-specific RNA endonuclease or a site-specific intron-like DNA endonuclease), lysis inhibition regulator, membrane protein, thymidine kinase, protein that contains a A1pp phosphatase motif, tRNA synthetase modifier (optionally a valyl-tRNA synthetase modifier), mRNA processing protein, UV repair enzyme (optionally a N-glycosylase UV repair enzyme), internal head protein (eg, a ipII internal head protein or a ipII internal head protein, Ip4 protein), endoribonuclease and DNA glycosylase (optionally a pyrimidine dimer DNA glycosylase);
   B. the synthetic phage genome comprises a deletion of one, more or all T4 genes of Table 7, or homologues or orthologues thereof;
   C. the synthetic phage genome comprises a deletion of T4 gene(s) (a) nrdC, (b) mobD, (c) rI, (d) rI.1, (e) tk, (f) vs, (g) regB and/or (h) denV, or a homologue or orthologue thereof; or
   D. the synthetic phage genome comprises a deletion of DNA between coordinates
      a) 2625 and 8092;
      b) 2668 and 7178;
      c) 8643 and 10313; or
      d) 9480 and 12224
      wherein the coordinates are the nucleotide positions in the direction from the pin gene towards the mobD and iPII genes of T4; or wherein homologous DNA from a T-even phage is deleted wherein said T-even phage is not a T4 phage.
48. The synthetic phage of any one of paragraphs 39 to 47, wherein the synthetic phage genome comprises a deletion of T4 genes tk, vs and regB, or homologues or orthologues thereof; optionally a deletion of DNA stretching from T4 gene nrdC to denV, or homologues or orthologues thereof.
49. The synthetic phage of any one of paragraphs 39 to 48, wherein the synthetic phage genome comprises a deletion of one or more genes, wherein
   A. each gene encodes a protein comprising an amino acid sequence selected from SEQ ID Nos: 1-128, or a homologue thereof; optionally wherein the homologue is an amino acid sequence that is at least 80% identical to said selected sequence; and/or
   B. each gene encodes an amino acid sequence selected from SEQ ID Nos: 1-42, or a homologue thereof; optionally wherein the homologue is an amino acid sequence that is at least 80% identical to said selected sequence.
50. The synthetic phage of any one of paragraphs 39 to 49, wherein the synthetic phage of (ii) is a T-even phage.
51. The synthetic phage of any one of paragraphs 39 to 50, wherein the synthetic phage is a lytic phage; and/or said phage that is not a T4 phage is a lytic phage.
52. A DNA comprising the genome of the synthetic phage of any one of paragraphs 39 to 51; optionally wherein the DNA is a chromosome of a bacterial cell or an episome (eg, a plasmid) comprised by a bacterial cell, such as a host cell of said synthetic phage.
53. The synthetic phage or DNA of any one of paragraphs 39 to 52, wherein the heterologous DNA comprises or encodes
   A. one or more components of a CRISPR/Cas system or a guided nuclease (eg, a Cas, TALEN, meganuclease or zinc finger); optionally wherein the heterologous DNA encodes a guide RNA (eg, a single guide RNA) and/or a Cas (eg, a Cas9, Cas3, Cas12, Cas13 or Cas14);
   B. an antibacterial agent;
   C. a phage tail fibre or component thereof;
   D. a vitamin;
   E. a blood protein;
   F. an antibody or fragment thereof; or
   G. a human or plant protein or fragment thereof.
54. The synthetic phage or DNA of any one of paragraphs 39 to 53, wherein said phage that is not a T4 phage is selected from the group consisting of the phages of Table 6, Escherichia phage T4, Escherichia phage T2, Escherichia phage T6,m Escherichia phage RB69, Shigella phage Shf125875, Escherichia phage APCEc01, Escherichia phage moskry, Escherichia phage ST0, Escherichia phage VB_EcoM_JS09, Shigella phage SP18, Escherichia phage vB_EcoM_PhAPEC2, Escherichia phage HX01, Salmonella phage SG1, Shigella phage pSs-1, Escherichia phage HY01, Yersinia phage PST, Escherichia phage AR1, Escherichia phage phiE142, Shigella phage SHFML-11, Escherichia phage slur07, Shigella phage SHFML-11, Escherichia phage UFV-AREG1, Escherichia phage vB_EcoM-UFV13, Shigella phage JK38, Shigella phage SHFML-26, Shigella phage Sf22, Escherichia phage ime09, Shigella phage S17, Yersinia phage phiD1, Escherichia phage RB3, Escherichia phage ECML-134, Escherichia phage vB_EcoM_ACG-C40, Escherichia phage vB_EcoM-fFiEo06, Escherichia phage PP01, Shigella phage Shfl2, Escherichia phage ECO4, Escherichia virus RB14, Escherichia phage vB_EcoM_JB75, Shigella phage Sf22, Escherichia phage VB_vPM_PD112, Shigella phage Sf23, Escherichia phage vB_EcoM_G2540, Escherichia phage vB_EcoM_G2133, Escherichia phage vB_EcoM_G4498, Escherichia virus RB32, Escherichia phage VB_EcoM_G4507, Escherichia phage vB_EcoM_G8, Escherichia phage EcNP 1, Enterobacteria phage RB27, Shigella virus KRT47, Escherichia phage teqdroes, Escherichia phage slur02, Yersinia phage fPS-90, Yersinia phage phiD1, Shigella phage Sf24 and Escherichia phage phiC120.
55. A method of producing synthetic phage particles, comprising
   (a) Allowing the production of synthetic phage in producer cells, wherein the phage are according to any one of paragraphs 39 to 51, 53 and 54; and
   (b) Isolating the phage; and
   (c) Optionally combining a population of said isolated synthetic phage with a pharmaceutically acceptable excipient. carrier or diluent to produce a pharmaceutical composition.
56. A method of producing a pharmaceutical composition, the method comprising combining a population of synthetic phage with a pharmaceutically acceptable excipient, carrier or diluent to produce a pharmaceutical composition, wherein the phage are according to any one of paragraphs 39 to 51, 53 and 54.
57. A population of synthetic phage according to any one of paragraphs 39 to 51, 53 and 54, or a pharmaceutical composition obtainable by the method of paragraph 18, for use as a medicament; optionally for administration to a human or animal subject for reducing infection by pathogenic host bacterial or archaeal cells or a first species or strain, wherein the phage are capable of infecting cells of said species or strain.
58. A synthetic phage, wherein the phage is
   (a) a synthetic Phi92 phage comprising a deletion of DNA from a Deletion Permissive Region (DPR) of the genome of the phage, wherein the region is between gene 39 and gene 46 or between gene 230 and gene 240; or
   (b) a synthetic version of a phage that is not a Phi92 phage, wherein the synthetic phage comprises a deletion of DNA from a region of its genome that is homologous or orthologous to said DPR of (i); and
wherein the synthetic phage is capable of replication in a host cell.
59. The synthetic phage of paragraph 58, wherein the deletion comprises up to 8000 bp of DNA.
60. The synthetic phage of paragraph 58 or 59, wherein the synthetic phage of (i) comprises an insertion of heterologous DNA, wherein the insertion is between genes 39 and 46 or between genes 230 and 240, or the synthetic phage of (ii) comprises an insertion of heterologous DNA, wherein the insertion is between a first gene and a second gene; wherein the first gene is homologous or orthologous to gene 39 of Phi92 and the second gene is homologous or orthologous to gene 46 of Phi92, or wherein the first gene is homologous or orthologous to gene 230 of Phi92 and the second gene is homologous or orthologous to gene 240 of Phi92.
61. The synthetic phage of paragraph 60, wherein the insertion comprises a total number (X) of base pairs of heterologous DNA, and (a) the deletion comprises a total number (Y) of base pairs of DNA wherein Y is at least 50% of X; or (b) the Phi92 phage or said phage that is not a Phi92 comprises a capsid that has a DNA packaging capacity of Zbp and the total number of base pairs of the genomic DNA of the synthetic phage is 90-110% of Z.
62. A synthetic phage, wherein the phage is
   (a) a synthetic Phi92 phage comprising an insertion of DNA into a Deletion Permissive Region (DPR) of the genome of the phage, wherein the region is between gene 39 and gene 46 or between gene 230 and gene 240; or
   (b) a synthetic version of a phage that is not a Phi92 phage, wherein the synthetic phage comprises an insertion of DNA into a region of its genome that is homologous or orthologous to said DPR of (i); and
wherein the synthetic phage is capable of replication in a host cell.
63. The synthetic phage of any one of paragraphs 60 to 62, wherein the insertion comprises up to 8000 bp of DNA.
64. The synthetic phage of any one of paragraphs 58 to 63, wherein the DPR of the Phi92 phage comprises contiguous DNA between gene 39 and gene 46 or between gene 230 and gene 240, wherein the contiguous DNA is at least 1000 bp in length; or wherein the DPR of the Phi92 phage comprises at least 100 bp of DNA between gene 39 and gene 46 or between gene 230 and gene 240.
65. The synthetic phage of any one of paragraphs 58 to 66, wherein the DPR of the Phi92 phage extends from gene 39 to gene 46 and/or from gene 230 to gene 240.
66. The synthetic phage of any one of paragraphs 58 to 65, wherein
   A. the synthetic phage genome comprises a deletion of a one or more genes, wherein each gene encodes a DNA methy lase; and/or
   B. the synthetic phage genome comprises a deletion of one, more or all Phi92 genes of Table 9, or homologues or orthologues thereof.
67. The synthetic phage of any one of paragraphs 58 to 66, wherein the synthetic phage genome comprises
   (a) a deletion in one or more Phi92 genes 235, 236, 237, 238, 239 and 240, or homologues or orthologues thereof; optionally a deletion of DNA stretching from genes 235-240 or 238-240, or homologues or orthologues thereof; or
   (b) a deletion of Phi92 genes 39-46 and/or 235-240, or homologues or orthologues thereof.
68. The synthetic phage of any one of paragraphs 58 to 67, wherein the synthetic phage of (ii) is a rV5 or a rV5-like phage.
69. The synthetic phage of any one of paragraphs 58 to 68, wherein the synthetic phage is a lytic phage; and/or said phage that is not a Phi92 phage is a lytic phage.
70. A DNA comprising the genome of the synthetic phage of any one of paragraphs 58 to 69. optionally wherein the DNA is a chromosome of a bacterial cell or an episome (eg, a plasmid) comprised by a bacterial cell, such as a host cell of said synthetic phage.
71. The synthetic phage or DNA of any one of paragraphs 58 to 70, wherein the heterologous DNA comprises or encodes
   A. one or more components of a CRISPR/Cas system or a guided nuclease (eg, a Cas, TALEN, meganuclease or zinc finger); optionally wherein the heterologous DNA encodes a guide RNA (eg, a single guide RNA) and/or a Cas (eg, a Cas9, Cas3, Cas12, Cas13 or Cas14);
   B. an antibacterial agent;
   C. a phage tail fibre or component thereof;
   D. a vitamin;
   E. a blood protein;
   F. an antibody or fragment thereof; or
   G. a human or plant protein or fragment thereof.
72. A method of producing synthetic phage particles, comprising
   (a) Allowing the production of synthetic phage in producer cells, wherein the phage are according to any one of paragraphs 58 to 69 and 71; and
   (b) Isolating the phage; and
   (c) Optionally combining a population of said isolated synthetic phage with a pharmaceutically acceptable excipient, carrier or diluent to produce a pharmaceutical composition.
73. A method of producing a pharmaceutical composition, the method comprising combining a population of synthetic phage with a pharmaceutically acceptable excipient, carrier or diluent to produce a pharmaceutical composition, wherein the phage are according to any one of paragraphs 58 to 69 and 71.
74. A population of synthetic phage according to any one of paragraphs 58 to 69 and 71, or a pharmaceutical composition obtainable by the method of paragraph 18, for use as a medicament; optionally for administration to a human or animal subject for reducing infection by pathogenic host bacterial or archaeal cells or a first species or strain, wherein the phage are capable of infecting cells of said species or strain.
75. A synthetic phage, wherein the phage is
   (a) a synthetic T4 phage that comprises a deletion of DNA from, and/or an insertion of heterologous DNA into, a region of the genome of the phage corresponding to a region between coordinates
   (i) 1887 and 8983;
   (ii) 2625 and 8092;
   (iii) 1904 and 8113;
   (iv) 2668 and 7178;
   (v) 7844 and 11117;
   (vi) 8643 and 10313;
   (vii) 8873 and 12826;
   (viii) 9480 and 12224;
   (ix) 8454 and 17479; or
   (x) 9067 and 16673;
   wherein coordinates are with reference to wild-type T4 phage genome (SEQ ID NO: 129); or
   (b) a synthetic version of a phage (eg, a T-even phage) that is not a T4 phage, wherein the synthetic phage comprises a deletion of DNA from a region of its genome that is homologous or orthologous to said region of (a);
   and
   wherein the synthetic phage is capable of replication in a host bacterial cell.
76. The synthetic phage of paragraph 75, wherein the deletion comprises up to 8000 bp of DNA.
77. A method of producing a synthetic phage, the method comprising
   (a) providing a heterologous DNA comprising an insert.
   (b) providing a first phage genomic DNA.
   (c) allowing homologous recombination between a first region of the genomic DNA and the heterologous DNA and allowing homologous recombination between a second region of the genomic DNA and the heterologous DNA,
   wherein the insert is inserted between said regions whereby a hybrid DNA is produced that encodes the genome of a synthetic phage; and
   wherein
   A:
   (i) the coordinates of the first region are 1887-2625 and the coordinates of the second region are 8092-8983;
   (ii) the coordinates of the first region are 1904-2668 and the coordinates of the second region are 7178-8113;
   (iii) the coordinates of the first region are 7844-8643 and the coordinates of the second region are 10313-11117;
   (iv) the coordinates of the first region are 8873-9480 and the coordinates of the second region are 12224-12826; or
   (v) the coordinates of the first region are 8454-9067 and the coordinates of the second region are 16673-17479;
   wherein the first phage is a T4 phage and the coordinates are with reference to wild-type T4 phage genome (SEQ ID NO: 129);
   or
   B: the first phage (eg, a T-even phage) is not a T4 phage, and wherein the first and second regions are regions of the first phage genome that are homologous or orthologous to said first and second regions of any one of A(i) to (v).
78. A synthetic phage obtainable by the method of paragraph 77; or a composition comprising a plurality of synthetic phages, wherein each phage is obtainable by the method of paragraph 77.
79. The synthetic phage, method or composition of any one of paragraphs 75 to 78, wherein the DNA insertion encodes one or more components of a CRISPR/Cas system: optionally wherein the DNA insertion encodes one or more different crRNAs or guide RNAs and/or encodes one or more Cas.
80. The synthetic phage, method or composition of any one of paragraphs 75 to 79, wherein the insertion comprises a total number (X) of base pairs of heterologous DNA, and (a) the deletion comprises a total number (Y) of base pairs of DNA wherein Y is at least 50% of X; or (b) the T4 phage or said phage that is not a T4 comprises a capsid that has a DNA packaging capacity of Zbp and the total number of base pairs of the genomic DNA of the synthetic phage is 90-110% of Z.
81. The synthetic phage, method or composition of any one of paragraphs 75 to 80, wherein the insertion comprises up to 8000 bp of DNA.
82. The synthetic phage, method or composition of any one of paragraphs 75 to 81, wherein the synthetic phage is a lytic phage; and/or said phage that is not a T4 phage is a lytic phage.
83. A DNA comprising the genome of the synthetic phage of any one of paragraphs 75, 76 and 78 to 82; optionally wherein the DNA is a chromosome of a bacterial cell or an episome (eg, a plasmid) comprised by a bacterial cell, such as a host cell of said synthetic phage.
84. The synthetic phage, method. composition or DNA of any one of paragraphs 75 to 83, wherein the DNA insertion comprises or encodes
   A. one or more components of a CRISPR/Cas system or a guided nuclease (eg, a Cas, TALEN, meganuclease or zinc finger); optionally wherein the heterologous DNA encodes a guide RNA (eg, a single guide RNA) and/or a Cas (eg, a Cas9, Cas3, Cas12, Cas13 or Cas14);
   B. an antibacterial agent;
   C. a phage tail fibre or component thereof;
   D. a vitamin;
   E. a blood protein;
   F. an antibody or fragment thereof; or
   G. a human or plant protein or fragment thereof.
85. The synthetic phage, method, composition or DNA of any one of paragraphs 75 to 85, wherein said phage that is not a T4 phage is selected from the group consisting of the phages of Table 6, Escherichia phage T4, Escherichia phage T2, Escherichia phage T6,m Escherichia phage RB69, Shigella phage Shfl25875, Escherichia phage APCEc01, Escherichia phage moskry, Escherichia phage ST0, Escherichia phage vB_EcoM_JS09, Shigella phage SP18, Escherichia phage vB_EcoM_PhAPEC2, Escherichia phage HX01, Salmonella phage SG1, Shigella phage pSs-1, Escherichia phage HY01, Yersinia phage PST, Escherichia phage AR1, Escherichia phage phiE142, Shigella phage SHFML-11, Escherichia phage slur07, Shigella phage SHFML-11, Escherichia phage UFV-AREG1, Escherichia phage vB_EcoM-UFV13, Shigella phage JK38, Shigella phage SHFML-26, Shigella phage Sf22, Escherichia phage ime09, Shigella phage S17, Yersinia phage phiD1, Escherichia phage RB3, Escherichia phage ECML-134, Escherichia phage vB3_EcoM_ACG-C40, Escherichia phage vB_EcoM-fFiEco06, Escherichia phage PP01, Shigella phage Shfl2, Escherichia phage ECO4, Escherichia virus RB14, Escherichia phage vB_EcoM_JB75, Shigella phage Sf22, Escherichia phage vB_vPM_PD112, Shigella phage Sf23, Escherichia phage vB_EcoM_G2540, Escherichia phage vB_EcoM_G2133, Escherichia phage vB_EcoM_G4498, Escherichia virus RB32. Escherichia phage vB_EcoM_G4507, Escherichia phage vB_EcoM_08, Escherichia phage EcNP 1, Enterobacteria phage RB27, Shigella virus KRT47, Escherichia phage teqdroes, Escherichia phage slur02, Yersinia phage fPS-90. Yersinia phage phiD1, Shigella phage Sf24 and Escherichia phage phiC120.
86. A method of producing synthetic phage particles, comprising
   (a) Allowing the production of synthetic phage in producer cells, wherein the phage are according to any one of paragraphs 75, 76 and 78 to 82, 83 and 85; and
   (b) Isolating the phage; and
   (c) Optionally combining a population of said isolated synthetic phage with a pharmaceutically acceptable excipient, carrier or diluent to produce a pharmaceutical composition.
87. A method of producing a pharmaceutical composition, the method comprising combining a population of synthetic phage with a pharmaceutically acceptable excipient, carrier or diluent to produce a pharmaceutical composition, wherein the phage are according to any one of paragraphs 75, 76 and 78 to 82, 83 and 85.
88. A population of synthetic phage according to any one of paragraphs 75, 76 and 78 to 82, 83 and 85, or a pharmaceutical composition obtainable by the method of paragraph 87, for use as a medicament; optionally for administration to a human or animal subject for reducing infection by pathogenic host bacterial or archaeal cells or a first species or strain, wherein the phage are capable of infecting cells of said species or strain.

### Generally Applicable Features:

Any cell herein may be a bacterial cell, archacal cell, algal cell, fungal cell, protozoan cell, invertebrate cell, vertebrate cell, fish cell, bird cell, mammal cell, companion animal cell, dog cell, cat cell, horse cell, mouse cell, rat cell, rabbit cell, eukaryotic cell, prokaryotic cell, human cell, animal cell, rodent cell, insect cell or plant cell. Preferably, the cell is a bacterial cell. Alternatively, the cell is a human cell.

Optionally, C1 and C2 is any Cas (eg, a Cas2, 3, 4, 5, or 6) of a Type I system. In this example, in an embodiment, the Cas may be fused or conjugated to a moiety that is operable to increase or reduce transcription of a gene comprising the target protospacer sequence. For example the nucleic acid encoding the Cas that is introduced into a cell may comprise a nucleotide sequence encoding the moiety, wherein the Cas and moiety are expressed in the host cell as a fusion protein. In one embodiment, the Cas is N-terminal of the moiety; in another embodiment it is C-terminal to the moiety.

In an example, a virus herein is a DNA virus, eg, ssDNA virus or dsDNA virus. In an example, a virus herein is a RNA virus.

Optionally, the hybrid DNA comprises encodes one or more Cascade proteins. For example, the hybrid DNA encodes a first Cas (C1) and/or a second Cas (C2) and the Cascade protein(s) are cognate with the C1 or C2, which is a Cas3.

Optionally, the hybrid DNA comprises encodes one or more Cascade proteins. For example, the hybrid DNA encodes a first Cas (C1) and/or a second Cas (C2) and Cas1 or Cas2 is a Cas3 that is cognate with Cascade proteins encoded by the cell.

Optionally, the Cascade proteins comprise or consist of cas5 (casD, csy2), cas6 (cas6f, cse3, casE), cas7 (csc2, csy3, cse4, casC) and cas8 (casA, cas8a1, cas8b1, cas8c, cas10d, cas8e, cse1, cas8f, csy1)

Optionally herein the hybrid DNA comprises a promoter and a Cas3-encoding or crRNA-encoding sequence that are spaced no more than 150, 100, 50, 40, 30, 20 or 10 bp apart, eg, from 30-45, or 30-40, or 39 or around 39 bp apart. Optionally herein a ribosome binding site and the Cas3-encoding or crRNA-encoding sequence are spaced no more than 20, 15, 14, 13, 12, 11, 10, 9, 8, 7, 6, 4 or 3 bp apart, eg, from 10-5, 6 or around 6 bp apart.

In an example, a promoter herein is in combination with a Shine-Dalgarno sequence comprising the sequence 5′-aaagaggagaaa-3′ (SEQ ID NO: 5) or a ribosome binding site homologue thereof. Optionally the promoter has an Anderson Score (AS) of AS≥0.5; or an Anderson Score (AS) of 0.5>AS>0.1; or an Anderson Score (AS) of ≤0.1.

Optionally, the hybrid DNA is devoid of nucleotide sequence encoding one, more or all of a Cas1, Cas2, Cas4, Cas6 (optionally Cas6f), Cas7 and Cas 8 (optionally Cas8f). Optionally, the hybrid DNA is devoid of a sequence encoding a Cas6 (optionally a Cas6f). Optionally, the hybrid DNA comprises (optionally in 5′ to 3′ direction) nucleotide sequence encoding one, more or all of Cas11, Cas7 and Cas8a1. Optionally, the hybrid DNA comprises nucleotide sequence encoding Cas3′ and/or Cas3″. In one embodiment, the hybrid DNA comprises nucleotide sequences (in 5′ to 3′ direction) that encode a Cas3 (eg, Cas3′ and/or Cas3″), Cas11, Cas7 and Cas8a1.

Optionally, a nucleotide sequence encoding Cas6 is between the Cas3 sequence(s) and the Cas11 sequence.

Optionally, the hybrid DNA comprises a Type IA CRISPR array or one or more nucleotide sequences encoding single guide RNA(s) (gRNA(s)), wherein the array and each gRNA comprises repeat sequence that is cognate with a Cas3. Thus, the array is operable in a host cell when the hybrid DNA has been introduced into the cell for production of guide RNAs, wherein the guide RNAs are operable with the Cas and Cascade proteins to target and modify (eg, cut) a target nucleotide sequence in the host cell, optionally thereby killing the cell. Similarly, single guide RNAs encoded by the hybrid DNA in one embodiment are operable with the Cas and Cascade proteins to target and modify (eg, cut) a target nucleotide sequence in the host cell, optionally thereby killing the cell.

Optionally, each cell comprises a Type IA CRISPR array that is cognate with the Cas3 (C1 or C2). Optionally, each cell comprises an endogenous Type IB, C, U, D, F or F CRISPR/Cas system. Optionally, the hybrid DNA comprises (optionally in 5′ to 3′ direction) nucleotide sequence encoding one, more or all of Cas8b1, Cas7 and Cas5. In one embodiment, the hybrid DNA comprises nucleotide sequences (in 5′ to 3′ direction) that encode a Cas3, Cas8b1, Cas7 and Cas5. Optionally, a nucleotide sequence encoding Cas6 is between the Cas3 sequence(s) and the Cas8b1 sequence Optionally, the hybrid DNA comprises a Type IB CRISPR array or one or more nucleotide sequences encoding single guide RNA(s) (gRNA(s)), wherein the array and each gRNA comprises repeat sequence that is cognate with the Cas3. Thus, the array is operable in a host cell when the hybrid DNA has been introduced into the cell for production of guide RNAs, wherein the guide RNAs are operable with the Cas and Cascade proteins to target and modify (eg, cut) a target nucleotide sequence in the host cell, optionally thereby killing the host cell. Similarly, single guide RNAs encoded by the hybrid DNA in one embodiment are operable with the Cas and Cascade proteins to target and modify (eg, cut) a target nucleotide sequence in the cell, optionally thereby killing the cell.

Optionally, the cell comprises a Type IB CRISPR array that is cognate with the Cas3. Optionally, the cell comprises an endogenous Type IA, C, U, D, F or F CRISPR/Cas system. Optionally, the hybrid DNA comprises (optionally in 5′ to 3′ direction) nucleotide sequence encoding one, more or all of Cas5, Cas8c and Cas7. In one embodiment, the hybrid DNA comprises nucleotide sequences (in 5′ to 3′ direction) that encode a Cas3, Cas5, Cas8c and Cas7. Optionally, a nucleotide sequence encoding Cas6 is between the Cas3 sequence(s) and the Cas5 sequence. Optionally, the hybrid DNA comprises a Type IC CRISPR array or one or more nucleotide sequences encoding single guide RNA(s) (gRNA(s)), wherein the array and each gRNA comprises repeat sequence that is cognate with the Cas3. Thus, the array is operable in a host cell when the hybrid DNA has been introduced into the cell for production of guide RNAs, wherein the guide RNAs are operable with the Cas and Cascade proteins to target and modify (eg. cut) a target nucleotide sequence in the cell, optionally thereby killing the cell. Similarly, the single guide RNAs encoded by the hybrid DNA in one embodiment are operable with the Cas and Cascade proteins to target and modify (eg, cut) a target nucleotide sequence in the cell, optionally thereby killing the cell.

Optionally, the host cell comprises a Type IC CRISPR array that is cognate with the Cas3. Optionally, the host cell comprises an endogenous Type IA, B, U, D, E or F CRISPR/Cas system. Optionally, the hybrid DNA comprises (optionally in 5′ to 3′ direction) nucleotide sequence encoding one, more or all of Cas8U2, Cas7, Cas5 and Cas6. In one embodiment, the hybrid DNA comprises nucleotide sequences (in 5′ to 3′ direction) that encode a Cas3, Cas8U2, Cas7, Cas5 and Cas6. Optionally, a nucleotide sequence encoding Cas6 is between the Cas3 sequence(s) and the Cas8U2 sequence.

Optionally, the hybrid DNA comprises a Type IU CRISPR array or one or more nucleotide sequences encoding single guide RNA(s) (gRNA(s)), wherein the array and each gRNA comprises repeat sequence that is cognate with the Cas3. Thus, the array is operable in a host cell when the hybrid DNA has been introduced into the cell for production of guide RNAs, wherein the guide RNAs are operable with the Cas and Cascade proteins to target and modify (eg, cut) a target nucleotide sequence in the cell, optionally thereby killing the cell. Similarly, the single guide RNAs encoded by the vector in one embodiment are operable with the Cas and Cascade proteins to target and modify (eg, cut) a target nucleotide sequence in the cell, optionally thereby killing the cell.

Optionally, the host cell comprises a Type IU CRISPR array that is cognate with the Cas3. Optionally, the host cell comprises an endogenous Type IA, B, C, D, E or F CRISPR/Cas system. Optionally, the vector comprises (optionally in 5′ to 3′ direction) nucleotide sequence encoding one, more or all of Cas10d, Cas7 and Cas5. Optionally, the hybrid DNA comprises a nucleotide sequence encoding Cas3′ and/or Cas3″. In one embodiment, the hybrid DNA comprises nucleotide sequences (in 5′ to 3′ direction) that encode a Cas3, Cas10d, Cas7 and Cas5. Optionally, a nucleotide sequence encoding Cas6 is between the Cas3 sequence(s) and the Cas10d sequence. Optionally, the hybrid DNA comprises a Type ID CRISPR array or one or more nucleotide sequences encoding single guide RNA(s) (gRNA(s)), wherein the array and each gRNA comprises repeat sequence that is cognate with the Cas3. Thus, the array is operable in a cell when the vector has been introduced into the cell for production of guide RNAs, wherein the guide RNAs am operable with the Cas and Cascade proteins to target and modify (eg, cut) a target nucleotide sequence in the cell, optionally thereby killing the cell. Similarly, the single guide RNAs encoded by the hybrid DNA in one embodiment are operable with the Cas and Cascade proteins to target and modify (eg, cut) a target nucleotide sequence in the cell, optionally thereby killing the cell.

Optionally, the cell comprises a Type ID CRISPR array that is cognate with the Cas3.

Optionally, the cell comprises an endogenous Type IA, B, C, U, E or F CRISPR/Cas system.

Optionally, the hybrid DNA comprises (optionally in 5′ to 3′ direction) nucleotide sequence encoding one, more or all of Cas8e, Cas11, Cas7, Cas5 and Cas6. In one embodiment, the hybrid DNA comprises nucleotide sequences (in 5′ to 3′ direction) that encode a Cas3, Cas8e, Cas11, Cas7, Cas5 and Cas6. Optionally, a nucleotide sequence encoding Cas6 is between the Cas3 sequence(s) and the Cas11 sequence. Optionally, the hybrid DNA comprises a Type IE CRISPR array or one or more nucleotide sequences encoding single guide RNA(s) (gRNA(s)), wherein the array and each gRNA comprises repeat sequence that is cognate with the Cas3. Thus, the array is operable in a host cell when the vector has been introduced into the cell for production of guide RNAs, wherein the guide RNAs are operable with the Cas and Cascade proteins to target and modify (eg, cut) a target nucleotide sequence in the cell, optionally thereby killing the cell. Similarly, the single guide RNAs encoded by the hybrid DNA in one embodiment are operable with the Cas and Cascade proteins to target and modify (eg, cut) a target nucleotide sequence in the cell, optionally thereby killing the cell.

Optionally, the cell comprises a Type IE CRISPR array that is cognate with the Cas3.

Optionally, the cell comprises an endogenous Type IA, B, C, D, U or F CRISPR/Cas system.

Optionally, the hybrid DNA comprises (optionally in 5′ to 3′ direction) nucleotide sequence encoding one, more or all of Cas8f, Cas5, Cas7 and Cas6f. In one embodiment, the hybrid DNA comprises nucleotide sequences (in 5′ to 3′ direction) that encode a Cas3, Cas8f, Cas5, Cas7 and Cas6f. Optionally, a nucleotide sequence encoding Cas6 is between the Cas3 sequence(s) and the Cas8f sequence. Optionally, the hybrid DNA comprises a Type IF CRISPR array or one or more nucleotide sequences encoding single guide RNA(s) (gRNA(s)), wherein the array and each gRNA comprises repeat sequence that is cognate with the Cas3. Thus, the array is operable in a cell when the vector has been introduced into the cell for production of guide RNAs, wherein the guide RNAs are operable with the Cas and Cascade proteins to target and modify (eg, cut) a target nucleotide sequence in the cell, optionally thereby killing the cell. Similarly, the single guide RNAs encoded by the hybrid DNA in one embodiment are operable with the Cas and Cascade proteins to target and modify (eg, cut) a target nucleotide sequence in the cell, optionally thereby killing the cell.

Optionally, the cell comprises a Type IF CRISPR array that is cognate with the Cas3.

Optionally, the cell comprises an endogenous Type IA, B, C, D, U or E CRISPR/Cas system.

Optionally, the Cas and Cascade are Type IA Cas and Cascade proteins.

Optionally, the Cas and Cascade are Type IB Cas and Cascade proteins.

Optionally, the Cas and Cascade are Type IC Cas and Cascade proteins.

Optionally, the Cas and Cascade are Type ID Cas and Cascade proteins.

Optionally, the Cas and Cascade are Type IE Cas and Cascade proteins.

Optionally, the Cas and Cascade are Type IF Cas and Cascade proteins.

Optionally, the Cas and Cascade are Type IU Cas and Cascade proteins.

Optionally, the Cas and Cascade are E coli (optionally Type IE or IF) Cas and Cascade proteins, optionally wherein the E co/i is ESBL-producing E. coli or E. coli ST131-O25b:H4.

Optionally, the Cas and Cascade are Clostridium (eg, C dificile) Cas and Cascade proteins, optionally C dificile resistant to one or more antibiotics selected from aminoglycosides, lincomycin, tetracyclines, erythromycin, clindamycin, penicillins, cephalosporins and fluoroquinolones.

Optionally, the Cas and Cascade are Pseudomonas aeruginosa Cas and Cascade proteins, optionally P aeruginosa resistant to one or more antibiotics selected from carbapenems, aminoglycosides, cefepime, ceftazidime, fluoroquinolones, piperacillin and tazobactam

Optionally, the Cas and Cascade are Klebsiella pneumoniae (eg, carbapenem-resistant Klebsiella pneumoniae or Extended-Spectrum Beta-Lactamase (ESBL)-producing K pneumoniae) Cas and Cascade proteins.

Optionally, the Cas and Cascade are E coli, C difficile, P aeruginosa, K pneumoniae, P furiosus or B halodurans Cas and Cascade proteins.

Optionally, each crRNAs or gRNAs comprises a spacer sequence that is capable of hybridising to a protospacer nucleotide sequence of the cell, wherein the protospacer sequence is adjacent a PAM, the PAM being cognate to the C1 or C2, wherein C1 or C2 is a Cas nuclease, eg, a Cas3. Thus, the spacer hybridises to the protospacer to guide the Cas3 to the protospacer. Optionally, the Cas3 cuts the protospacer, eg, using exo- and/or endonuclease activity of the Cas3. Optionally, the Cas3 removes a plurality (eg, at least 2, 3, 4, 5, 6, 7, 8, 9 or 10) nucleotides from the protospacer.

It will be understood that particular embodiments described herein are shown by w ay of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the an to which this invention pertains. All publications and patent applications and all US equivalent patent applications and patents are herein incorporated by reference to the same extent as if each individual publication or patent application was specifically and individually indicated to be incorporated by reference. Reference is made to the publications mentioned herein and equivalent publications by the US Patent and Trademark Office (USPTO) or WIPO, the disclosures of which are incorporated herein by reference for providing disclosure that may be used in the present invention and/or to provide one or more features (eg, of a vector) that may be included in one or more claims herein.

The use of the word “a” or “an” when used in conjunction with the term “comprising” in the claims and/or the specification may mean “one,” but it is also consistent with the meaning of “one or more,” “at least one.” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” or similar as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non-limiting Examples.

### EXAMPLES

### Example 1

### Executive Summary

Lytic viruses (such as lytic bacteriophages) take over the machinery of the cell to replicate themselves. They then lyse the cell, releasing newly synthesised phage particles. However, not all infection events lead to successful viral replication and host cell lysis. Therefore, to increase the killing potential of lytic viruses (using lytic T-even bacteriophages as an exemplary model), we engineer their DNA by adding a CRISPR system that targets the host. The process of insertion of a functional CRISPR system into the phage or virus genome is called CRISPR arming.

### Introduction

Bacteriophages are among the most abundant and diverse entities in the biosphere. They are composed of proteins that encapsulate a DNA or RNA genome and may have structures of various complexities. Bacteriophage genomes may encode as few as four genes and as many as hundreds of genes. Bacteriophages with larger genomes tend to have a broader host range and better chance to evade host defense systems.

We have developed a toolbox for CRISPR arming of lytic bacteriophages. The tools developed can be applied to a wide range of bacteriophages or other viruses with minor system specific modifications.

### Study Objectives

Objective 1: Development of an engineering platform that harnesses the natural recombination system of the target phage
Objective 2: Engineering of phage genomes using synthetic DNA fragments
Objective 3: Bacteriophage genome assembly from synthetic DNA fragments

### Materials and Methods

### Strains and Culture Conditions

Unless otherwise stated, bacteria were cultivated at 37° C. in lysogeny broth (LB) or its enriched version (2×YT) at 250 rpm in liquid media, or on agar plates containing 1.5% (wt/vol) agar. When necessary. cultures were supplemented with antibiotics: tetracycline (10 μg/ml), spectinomycin (400 μg/ml), ampicillin (100 μg/ml).

### Plasmid and Strain Construction

Plasmids were constructed using PCR generated fragments.

### Phage Propagation

Phage lysates were produced in 2×YT supplemented with 5 mM CaCl₂ and 10 mM MgSO₄. A single phage plaque was added to 10 ml broth containing 100 μl overnight cells. The culture was incubated until clear lysis of the culture was observed. The lysate was centrifuged at 4000 g for 10 minutes and filtered through a 0.2 μm cartridge. Lysates were stored at 4° C. The titer of phages was determined by preparing a serial dilution and spotting the dilutions on a double layer agar.

Double layer agar plates were prepared by overlaying an LB plate (containing the appropriate antibiotics if needed) with 3 ml of soft agar containing 100 μl of an overnight cell culture.

### Results

### CRISPR Arming of T-Even Family Phages

### Genomic Organization of T-Even Phages

T-even phages are a group of double-stranded DNA bacteriophages. They are large and highly complex viruses containing many genes. Some members of the T-even family served as paradigm systems in molecular biology and therefore their structure, genetic organization, function, and interaction with the host cell is well understood.

An important feature of the T-even group is that the phage head is capable of containing a DNA molecule larger than the complete genome and packaging of DNA into phage heads is determined by the headful mechanism. Therefore the packaged DNA is terminally redundant, i.e. the two ends of the DNA contain an identical sequence which constitutes about 3% of the unit genome. Because the initiation of DNA packaging into the phage head is not confined to a specific DNA sequence, these phages also show circular permutation. The third key feature is that T-even phages possess a highly efficient homologous recombination system. In our experience this recombination system requires only a few hundred base pair homologous region, tolerates several mismatches in these regions, and is independent of the general homologous recombination system of E. coli (RecA pathway).

We thought it important to retain essential genes. We retained the genes that are required for replication of phage DNA, for synthesis of structural components, for the assembly of the phage particle, and lysis of the host cell.

### Identification of Potentially Removable Regions

CRISPR arming of a phage with arrays and Cas genes requires the insertion of about 8000 bp DNA into the phage chromosome. Addition of such a large DNA fragment would make the chromosome larger than would tit into the phage head. Therefore, DNA needs to be removed; that is, certain phage genes need to be deleted. We considered which DNA, therefore, might be permissive for deletion and yet still produce a viable phage (ie, a CRISPR armed phage that can infect host target cells).

We decided to avoid phage genome regions required for DNA modification or host DNA degradation. Although these functions are technically not essential for phage propagation on standard laboratory hosts, we wanted to maintain them in a phage aimed for therapeutic purposes. The function of DNA modification enhances the host range and propagation efficiency, which is advantageous for therapeutic use, while host DNA degradation prevents transduction of host DNA, i.e., transfer of genes from one host cell to another, including virulence and antibiotic resistance genes. These considerations lead us to identify a region for deletion (Deletion Permissive Region, DPR), between the pin (protease inhibitor) gene and the internal protein gene iPII.

The Bacteriophage T4 genes that belong to the DPR region are listed in Table 7 Considering the positions of genes with known functions (boldface type in Table 7), we followed two different approaches for the removal of genes. In the first approach, we inserted the CRISPR system in two steps, removing two separate sets of genes at the same time. In the second approach we created a system that allows arming of the phage with a fully functional CRISPR system in a single step. With the second approach we could replace different parts of the DPR region with the CRISPR system and compare the performance of the phages obtained. That is, we could learn how the different genes with unknown functions affect the performance of the phage.

### Construction of Recombination Donor Sequences

Recombination donor sequences have relatively simple structures. They are assembled from four DNA fragments carrying the following elements: (i) plasmid replication origin and selectable marker, (ii) upstream homologous sequence (UHS), (iii) Cargo (CRISPR/Cas system sequence) to be inserted into the phage genome, and (iv) downstream homologous sequence (DHS). The sequences were assembled into a circular plasmid in the above order (i-ii-iii-iv). The CRISPR element (cargo) to be inserted into the phage chromosome was flanked by the upstream (UHS) and downstream (DHS) homologous regions in selectable plasmids. The orientation of transcription for UHS, CRISPR, and DHS followed that of the phage (right to left). All cargo sequences were derived from the Type I-E Escherichia coli CRISPR-Cas system. Three different cargo elements were constructed. In the two-step arming strategy CRISPR arrays (containing multiple spacers targeting E. coli chromosome genes) and cas genes (Cas3 and CasA, B, C, D and E) were maintained and transferred to the phage chromosome (ie, the DNA molecule encompassing the phage genome), separately—i.e, firstly the CRISPR array was integrated into the phage chromosome during one engineering cycle and subsequently, the cas genes were integrated into the phage chromosome in a second engineering cycle. In the single-step method complete CRISPR systems (ie, CRISPR arrays and cas genes) were constricted which were maintained in cloning bacterial cell strains where the cutting target sites were mutated (so not to be targeted by CRISPR).

Our recombination donor sequences were based on the CloDF13 replication origin and a spectinomycin resistance marker. The UHS, DHS, and cargo (CRISPR) sequences in the recombination donor plasmids used are listed in Table 8(a).

### Recombination of CRISPR Systems to the Phage Chromosome

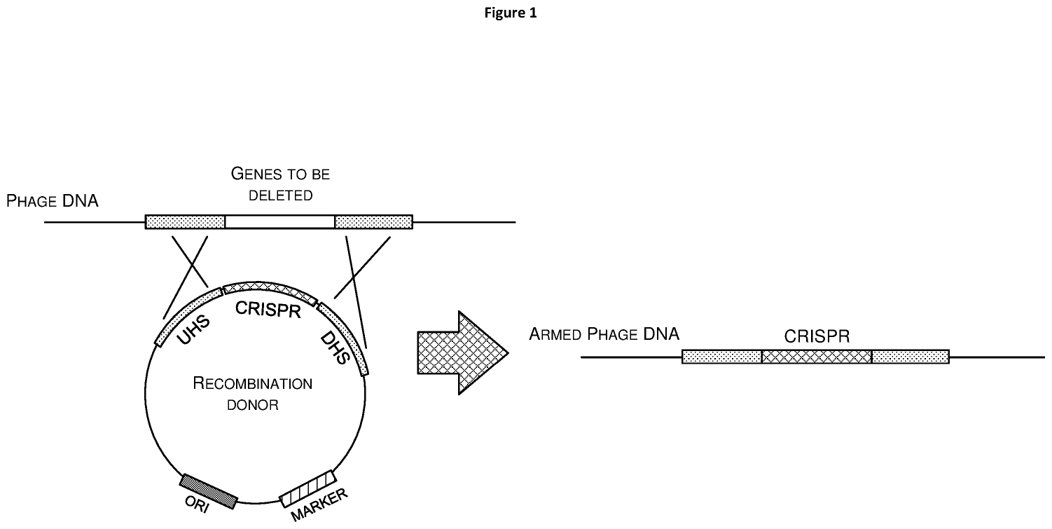

Recombination of the CRISPR components to the phage chromosome occurs by two homologous recombination reactions. One is between the UHS and its homologous sequence on the phage DNA, and the other is between the DHS and its homologous sequence on the phage DNA, as depicted in FIG. 1. Recombination is mediated by the phage's homologous recombination system Recombination of the UHS and DHS with their homologous sequences on the phage chromosome resulted in a CRISPR armed phage in which a piece of the phage chromosome was replaced by the CRISPR system.

To carry out the recombination reaction, the recombination donor plasmid was transferred to a bacterial cloning strain that is susceptible to the phage that we wanted to CRISPR arm. Subsequently, cells carrying the recombination plasmid are infected with the phage at low multiplicity, that is, the initial number of phages is much less than the number of cells in the culture. When the phage replicates in the cells, recombination occurs between the phage and the recombination plasmid at a certain rate, resulting in a mixed progeny of wild type and recombinant phages. The rate of recombination and thus the fraction of recombinant phages in the progeny primarily depend on the lengths of UHS and DHS and the copy number of the recombinant donor plasmid.

We have developed two different methods for CRISPR arming phages.

In the first method we recombined the arrays and the Cas genes into the phage chromosome in two separate steps. This way we split the CRISPR system to two parts, and therefore we do not need to protect the cells that are used for the engineering of the phage from the harmful attack of the CRISPR system.

In the second method we first re-engineered the chromosome of the bacterial strain used for engineering the phage. We removed all the sites on the chromosome that would be attacked by the CRISPR system with which we want to arm the phage. Once the re-engineered bacterial cells were available, we can could recombination donor plasmids that carry complete CRISPR systems. That is, phages could be CRISPR armed in a single step.

### Selection of Recombinant CRISPR Armed Phages

The recombination process results in a mixed phage progeny containing both wild type and CRISPR armed phages. Therefore we needed a selection system that is able to enrich the CRISPR armed version. A highly efficient method for this purpose is CRISPR-Cas mediated counter-selection (Hatoum-Aslan, 2018).

CRISPR arming of SA116 and SA117

CRISPR arming of phage SA116 and SA117 was performed in a similar way, first inserting the arrays and then the cas genes. Single plaques were selected and phages were amplified in 10 ml cell culture. The engineered region was PCR amplified using primers that anneal to the phage DNA upstream and downstream of the insertion site of the arrays. For the insertion of the cas genes, we used plasmids that contain the cas3, casA, casB, casC, casD, and casE genes in a single transcription unit. In this step we chose to remove the rl lysis inhibition gene because deletion of this gene was reported to result in faster lysis and larger plaque sizes (Burch et al. 2011). Recombinant phages were counter-selected on relevant strains. Single plaques were selected and phages were amplified in 10 ml cell culture. The engineered regions were PCR amplified using three primer pairs and the sequence of PCR products was verified Next, DNA was extracted from the phage lysates and the genome of the CRISPR armed phages was determined by next generation sequencing. The armed phages were named SA116.1 and SA117.1.

Plasmids used differed only in the 32-bp spacer sequence that was identical to the target sequence. Plasmids were based on the pSC101 replication origin and a tetracycline resistance marker. They carried a constitutively expressed E. coli Cas operon and a single spacer array from a separate constitutive promoter.

### Construction of a Deletion-Scanning Library of CRISPR Armed SA117 Phages

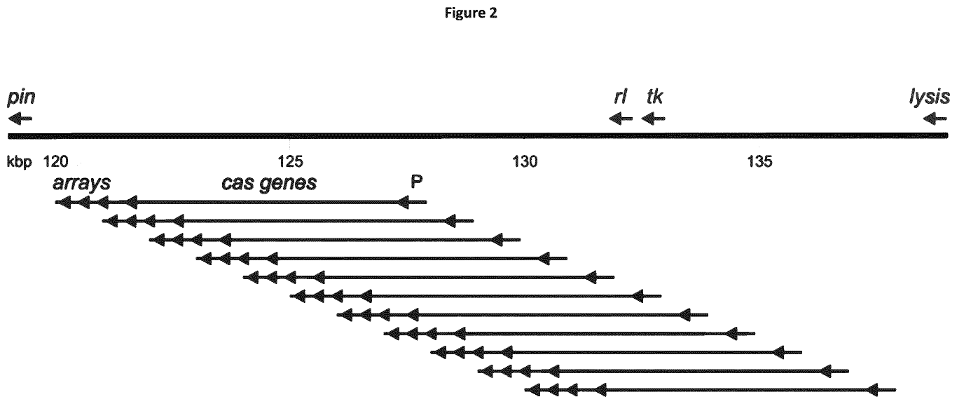

In order to speed up the CRISPR arming process we developed a method for transferring a fully functional CRISPR system to the phage chromosome in a single step. This method required construction of strains which lack the target sequences of the CRISPR systems used. Recombination donor plasmids had the same overall structure as shown in FIG. 1. These plasmids carried the E. coli cas3, casA, casB, casC, casD, and casE genes followed by an array targeting a set of conserved E. coli sequences. The whole unit was transcribed from a single promoter region upstream of cas3 (FIG. 2).

To identify the optimal location of the CRISPR cassette in the DPR region, we constructed a set of recombination donor plasmids which carried the same cargo sequence but differed in the UHS and DHS sequencing determining the site of insertion (FIG. 2). Eleven plasmids were designed to cover the region between the pin and lysis genes. After creating the armed phages, we can compare their performance in different conditions and understand if any of the genes in the DPR region contribute to the fitness, host range, manufacturing properties, stability, in vivo performance, etc. of SA117. Successful phages were chosen, which after engineering (integration of the CRISPR-Cas cassette) had retained its infective spectrum against a representative panel of clinical isolates, measured by liquid growth curve assays (data not shown).

### CRISPR Arming of DTR Phages

### Genome Structure of DTR Phages

DTR phages are characterized by the Direct Terminal sequence Repeats that mark the beginning and the end of the phage genome. That is, the packaged DNA is identical in each phage particle, flanked by the terminal repeats. The advantage of these phages is that they possess a sequence specific DNA packaging mechanism and therefore generally do not transduce host genes. The rv5-like group of DTR phages (see, eg. Kropinski, A. M. et al, The host-range, genomics and proteomics of Escherichia coli O157: H7bacteriophage rv5. Vim. J 2013, 10, 76; and Joanna Kaczorowska et al, A Quest of Great Importance-Developing a Broad

Spectrum Escherichia coli Phage Collection, Viruses 2019, 11, 899. doi:10 3390/v11100899) contains complex phages with large genome sizes. Some of the members of the rv5-like group are well characterized broad host range phages, such as Phi92. In phi92 genes are clustered in at least five transcriptional units, which lie alternately on the direct and complementary strands (Schwarzer et al, 2012).

### Identification of Removable Regions

Assuming that the phage head can tolerate extra DNA accounting to up to 5% of the genome size, we identified a set of disposable genes in Phi92. Phi92 has not been subjected to extensive deletion analysis. Therefore, we needed an alternative approach to identify regions for insertion of the CRISPR system First we performed BLAST analysis to compare the Phi92 genome to genomes of its close relatives in the databases (GenBank+EMBL+DDBJ+PDB+RefSeq). This analysis allowed us to identify two longer stretches of potentially disposable genes (Deletion Permissive Regions, DPRs), around genes 39 to 46 and 230 to 240 (Table 9). In the latter region we found two natural deletions, affecting genes 235-240 (deletion M2) and genes 238-240 (deletion M5). Based on this analysis, our first approach for CRISPR arming Phi92 was to insert the cas genes in place of genes 39 to 46, and to replace genes 235 to 240 by the CRISPR arrays.

### Recombination of CRISPR Systems to the Phage Chromosome

CRISPR arming of Phi92 was performed by using synthetic DNA fragments and the homologous recombination system of Bacteriophage λ (Red, recombination deficient). The advantage of the Red system compared to the general recombination system of E. coli is that it requires only very short homologies between the recombination partners. That is, recombination donor sequences can be constructed by PCR, and the required homologous sequences (about 50 bp) can be added to the primers.

The Phi92 chromosome was CRISPR armed in two steps. We constructed a set of template plasmids carrying the cas genes of the E. coli CRISPR system, and another set that carried arrays carrying 3 to 5 spacer sequences (Table 10). Selected CRISPR components were PCR amplified and integrated into the phage genome.

### Selection of Recombinant (CRISPR Armed) Phages

The recombineering process used for engineering Phi92 resulted in a mixed phage progeny containing both wild type and CRISPR armed phages. We used a counter-selection system. Recombinant phages were selected on Stellar cells carrying a plasmid borne CRISPR system targeting phage gene(s) replaced in the recombinant phages. We tested plaques by PCR before producing a lysate. Positive plaques were amplified in 10 ml cell culture. The engineered regions were PCR amplified using three primer pairs and the sequence of PCR products was verified. Next, DNA is extracted from the phage lysates and the genome of the CRISPR armed phages is determined by next generation sequencing. Successful phages were chosen, which after engineering (integration of the CRISPR was cassette) had retained its infective spectrum against a representative panel of clinical isolates, measured by liquid growth curve assays (data not shown).

### DISCUSSION

We identified Deletion Permissive Regions that we determined to be permissive for deletion of DNA and insertion of heterologous DNA (in this case components of CRISPR/Cas systems). Using this finding we were able to successfully delete DNA from DPRs in T-even and Phi92 phage and to arm these phage with CRISPR arrays and Cas-encoding sequences. Phages were thereby produced that were able to retain infectivity for desired bacterial strains and species. Deletion and insertion sizes for representative phages (Phages 1-5) are shown in Table 8(b).

Phages 1-3 were based on T-even phage. Table 8(a) lists the plasmids used as templates for recombination to produce such phages. The genomic content between UHS and DHS varied, consequently the size of the deletion in the different phages as well.

Phage 1 was constructed in two steps: First the array was added with p958, adding 1132 bp and removing 5561 bp. In the second step the cas genes were added with p948, adding 7233 bp and removing 2940 bp. Phage 3 was constructed in a similar way, first adding the array with p902 and then the cas genes with p940. Phage 2 was made in a single step with p996.

Phage 4 and 5 were based on Phi92 phage. Phage 4 had only the cas genes inserted, and Phage 5 was made from Phage 4 by adding the array.

### REFERENCES

Datsenko K A, Wanner B L. (2000). One-step inactivation of chromosomal genes in Escherichia coli K-12 using PCR products. Proc Natl Acad Sci USA. 97(12), 6640-5.
Kutter, E. et al (2018). From Host to Phage Metabolism: Hot Tales of Phage T4's Takeover of E. coli. Viruses, 10(7), 387.
Hatoum-Aslan A. (2018). Phage Genetic Engineering Using CRISPR-Cas Systems. Viruses. 10(6), 335.
Vlot, M. et al (2018). Bacteriophage DNA glucosylation impairs target DNA binding by type I and II but not by type V CRISPR-Cas effector complexes. Nucleic Acids Res, 46(2), 873-885.
Dharmalingam, K. et al (1982). Physical mapping and cloning of bacteriophage T4 anti-restriction endonuclease gene. J Bacteriol. 149(2):694-9.
Schwarzer, D., et al (2012) A multivalent adsorption apparatus explains the broad host range of phage phi92: a comprehensive genomic and structural analysis. J Virol, 86(19), 10384-10398.
Wang, H. H., et al (2009). Programming cells by multiplex genome engineering and accelerated evolution. Nature, 460(7257), 894-898.
Burch, L. H., et al (2011). The bacteriophage T4 rapid-lysis genes and their mutational proclivities. Journal of bacteriology, 193(14), 3537-3545.

TABLE 1
Example Bacteria
Optionally, the cell or cells are cell(s) of a genus or species selected from this Table.

| | | | | |
|---|---|---|---|---|
| Abiotrophia | Acidocella | Actinomyces | Alkalilimnicola | Aquaspirillum |
| Abiotrophia defectiva | Acidocella aminolytica | Actinomyces bovis | Alkalilimnicola ehrlichii | Aquaspirillum polymorphum |
| Acaricomes | Acidocella facilis | Actinomyces denticolens | Alkaliphilus | Aquaspirillum |
| Acaricomes phytoseiuli | Acidomonas | Actinomyces europaeus | Alkaliphilus oremlandii | putridiconchylium |
| Acetitomaculum | Acidomonas methanolica | Actinomyces georgiae | Alkaliphilus transvaalensis | Aquaspirillum serpens |
| Acetitomaculum ruminis | Acidothermus | Actinomyces gerencseriae | Allochromatium | Aquimarina |
| Acetivibrio | Acidothermus cellulolyticus | Actinomyces | Allochromatium vinosum | Aquimarina latercula |
| Acetivibrio cellulolyticus | Acidovorax | hordeovulneris | Alloiococcus | Arcanobacterium |
| Acetivibrio ethanolgignens | Acidovorax anthurii | Actinomyces howellii | Alloiococcus otitis | Arcanobacterium |
| Acetivibrio multivorans | Acidovorax caeni | Actinomyces hyovaginalis | Allokutzneria | haemolyticum |
| Acetoanaerobium | Acidovorax cattleyae | Actinomyces israelii | Allokutzneria albata | Arcanobacterium pyogenes |
| Acetoanaerobium noterae | Acidovorax citrulli | Actinomyces johnsonii | Altererythrobacter | Archangium |
| Acetobacter | Acidovorax defluvii | Actinomyces meyeri | Altererythrobacter | Archangium gephyra |
| Acetobacter aceti | Acidovorax delafieldii | Actinomyces naeslundii | ishigakiensis | Arcobacter |
| Acetobacter cerevisiae | Acidovorax facilis | Actinomyces neuii | Altermonas | Arcobacter butzleri |
| Acetobacter cibinongensis | Acidovorax konjaci | Actinomyces odontolyticus | Altermonas haloplanktis | Arcobacter cryaerophilus |
| Acetobacter estunensis | Acidovorax temperans | Actinomyces oris | Altermonas macleodii | Arcobacter halophilus |
| Acetobacter fabarum | Acidovorax valerianellae | Actinomyces radingae | Alysiella | Arcobacter nitrofigilis |
| Acetobacter ghanensis | Acinetobacter | Actinomyces slackii | Alysiella crassa | Arcobacter skirrowii |
| Acetobacter indonesiensis | Acinetobacter baumannii | Actinomyces turicensis | Alysiella filiformis | Arhodomonas |
| Acetobacter lovaniensis | Acinetobacter baylyi | Actinomyces viscosus | | Arhodomonas aquaeolei |
| Acetobacter malorum | Acinetobacter bouvetii | Actinoplanes | Aminobacter | Arsenophonus |
| Acetobacter nitrogenifigens | Acinetobacter calcoaceticus | Actinoplanes auranticolor | Aminobacter aganoensis | Arsenophonus nasoniae |
| Acetobacter oeni | Acinetobacter gerneri | Actinoplanes brasiliensis | Aminobacter aminovorans | |
| Acetobacter orientalis | Acinetobacter haemolyticus | Actinoplanes consettensis | Aminobacter niigataensis | Arthrobacter |
| Acetobacter orleanensis | Acinetobacter johnsonii | Actinoplanes deccanensis | Aminobacterium | Arthrobacter agilis |
| Acetobacter pasteurianus | Acinetobacter junii | Actinoplanes derwentensis | Aminobacterium mobile | Arthrobacter albus |
| Acetobacter pornorurn | Acinetobacter lwoffi | Actinoplanes digitatis | Aminomonas | Arthrobacter aurescens |
| Acetobacter senegalensis | Acinetobacter parvus | Actinoplanes durhamensis | Aminomonas paucivorans | Arthrobacter |
| Acetobacter xylinus | Acinetobacter radioresistens | Actinoplanes ferrugineus | Ammoniphilus | chlorophenolicus |
| Acetobacterium | Acinetobacter schindleri | Actinoplanes globisporus | Ammoniphilus oxalaticus | Arthrobacter citreus |
| Acetobacterium bakii | Acinetobacter soli | Actinoplanes humidus | Ammoniphilus oxalivorans | Arthrobacter crystallopoietes |
| Acetobacterium carbinolicum | Acinetobacter tandoii | Actinoplanes italicus | Amphibacillus | Arthrobacter cumminsii |
| Acetobacterium dehalogenans | Acinetobacter tjernbergiae | Actinoplanes liguriensis | Amphibacillus xylanus | Arthrobacter globiformis |
| Acetobacterium fimetarium | Acinetobacter towneri | Actinoplanes lobatus | Amphritea | Arthrobacter |
| Acetobacterium malicum | Acinetobacter ursingii | Actinoplanes missouriensis | Amphritea balenae | histidinolovorans |
| Acetobacterium paludosum | Acinetobacter venetianus | Actinoplanes palleronii | Amphritea japonica | Arthrobacter ilicis |
| Acetobacterium tundrae | Acrocarpospora | Actinoplanes philippinensis | Amycolatopsis | Arthrobacter luteus |
| Acetobacterium wieringae | Acrocarpospora corrugata | Actinoplanes rectilineatus | Amycolatopsis alba | Arthrobacter methylotrophus |
| Acetobacterium woodii | Acrocarpospora | Actinoplanes regularis | Amycolatopsis albidoflavus | Arthrobacter mysorens |
| Acetofilamentum | macrocephala | Actinoplanes | Amycolatopsis azurea | Arthrobacter nicotianae |
| Acetofilamentum rigidum | Acrocarpospora | teichomyceticus | Amycolatopsis coloradensis | Arthrobacter nicotinovorans |
| Acetohalobium | pleiomorpha | Actinoplanes utahensis | Amycolatopsis lurida | Arthrobacter oxydans |
| Acetohalobium arabaticum | | | Amycolatopsis mediterranei | Arthrobacter pascens |
| Acetomicrobium | Actibacter | Actinopolyspora | Amycolatopsis rifamycinica | Arthrobacter |
| Acetomicrobium faecale | Actibacter sediminis | Actinopolyspora halophila | Amycolatopsis rubida | phenanthrenivorans |
| Acetomicrobium flavidum | Actinoalloteichus | Actinopolyspora | Amycolatopsis sulphurea | Arthrobacter |
| Acetonema | Actinoalloteichus | mortivallis | Amycolatopsis tolypomycina | polychromogenes |
| Acetonema longum | cyanogriseus | Actinosynnema | Anabaena | Atrhrobacter protophormiae |
| Acetothermus | Actinoalloteichus | Actinosynnema mirum | Anabaena cylindrica | Arthrobacter |
| Acetothermus paucivorans | hymeniacidonis | Actinotalea | Anabaena flos-aquae | psychrolactophilus |
| Acholeplasma | Actinoalloteichus spitiensis | Actinotalea fermentans | Anabaena variabilis | Arthrobacter ramosus |
| Acholeplasma axanthum | Actinobaccillus | Aerococcus | Anaeroarcus | Arthrobacter sulfonivorans |
| Acholeplasma brassicae | Actinobacillus capsulatus | Aerococcus sanguinicola | Anaeroarcus burkinensis | Arthrobacter sulfureus |
| Acholeplasma cavigenitalium | Actinobacillus delphinicola | Aerococcus urinae | Anaerobaculum | Arthrobacter uratoxydans |
| Acholeplasma equifetale | Actinobacillus hominis | Aerococcus urinaeequi | Anaerobaculum mobile | Arthrobacter ureafaciens |
| Acholeplasma granularum | Actinobacillus indolicus | Aerococcus urinaehominis | Anaerobiospirillum | Arthrobacter viscosus |
| Acholeplasma hippikon | Actinobacillus lignieresii | Aerococcus viridans | Anaerobiospirillum | Arthrobacter woluwensis |
| Acholeplasma laidlawii | Actinobacillus minor | Aeromicrobium | succiniciproducens | Asaia |
| Acholeplasma modicum | Actinobacillus muris | Aeromicrobium erythreum | Anaerobiospirillum thomasii | Asaia bogorensis |
| Acholeplasma morum | Actinobacillus | Aeromonas | Anaerococcus | Asanoa |
| Acholeplasma multilocale | pleuropneumoniae | Aeromonas | Anaerococcus hydrogenalis | Asanoa ferruginea |
| Acholeplasma oculi | Actinobacillus porcinus | allosaccharophila | Anaerococcus lactolyticus | Asticcacaulis |
| Acholeplasma palmae | Actinobacillus rossii | Aeromonas bestiarum | Anaerococcus prevotii | Asticcacaulis biprosthecium |
| Acholeplasma parvum | Actinobacillus scotiae | Aeromonas caviae | Anaerococcus tetradius | Asticcacaulis excentricus |
| Acholeplasma pleciae | Actinobacillus seminis | Aeromonas encheleia | Anaerococcus vaginalis | Atopobacter |
| Acholeplasma vituli | Actinobacillus succinogenes | Aeromonas | | Atopobacter phocae |
| Achromobacter | Actinobaccillus suis | enteropelogenes | Anaerofustis | Atopobium |
| Achromobacter denitrificans | Actinobacillus ureae | Aeromonas eucrenophila | Anaerofustis stercorihominis | Atopobium fossor |
| Achromobacter insolitus | Actinobaculum | Aeromonas ichthiosmia | Anaeromusa | Atopobium minutum |
| Achromobacter piechaudii | Actinobaculum massiliense | Aeromonas jandaei | Anaeromusa acidaminophila | Atopobium parvulum |
| Achromobacter ruhlandii | Actinobaculum schaalii | Aeromonas media | Anaeromyxobacter | Atopobium rimae |
| Achromobacter spanius | Actinobaculum suis | Aeromonas popoffii | Anaeromyxobacter | Atopobium vaginae |
| Acidaminobacter | Actinomyces urinale | Aeromonas sobria | dehalogenans | Aureobacterium |
| Acidaminobacter | Actinocatenispora | Aeromonas veronii | Anaerorhabdus | Aureobacterium barkeri |
| hydrogenoformans | Actinocatenispora rupis | Agrobacterium | Anaerorhabdus furcosa | Aurobacterium |
| Acidaminococcus | Actinocatenispora | Agrobacterium | Anaerosinus | Aurobacterium liquefaciens |
| Acidaminococcus fermentans | thailandica | gelatinovorum | Anaerosinus glycerini | Avibacterium |
| Acidaminococcus intestini | Actinocatenispora sera | Agrococcus | Anaerovirgula | Avibacterium avium |
| Acidicaldus | Actinocorallia | Agrococcus citreus | Anaerovirgula multivorans | Avibacterium gallinarum |
| Acidicaldus organivorans | Actinocorallia aurantiaca | Agrococcus jenensis | Ancalomicrobium | Avibacterium paragallinarum |
| Acidimicrobium | Actinocorallia aurea | Agromonas | Ancalomicrobium adetum | Avibacterium volantium |
| Acidimicrobium ferrooxidans | Actinocorallia cavernae | Agromonas oligotrophica | Ancylobacter | Azoarcus |
| Acidiphilium | Actinocorallia glomerata | Agromyces | Ancylobacter aquaticus | Azoarcus indigens |
| Acidiphilium acidophilum | Actinocorallia herbida | Agromyces fucosus | Aneurinibacillus | Azoarcus tolulyticus |
| Acidiphilium angustum | Actinocorallia libanotica | Agromyces hippuratus | Aneurinibacillus | Azoarcus toluvorans |
| Acidiphilium cryptum | Actinocorallia longicatena | Agromyces luteolus | aneurinilyticus | Azohydromonas |
| Acidiphilium multivorum | Actinomadura | Agromyces mediolanus | Aneurinibacillus migulanus | Azohydromonas australica |
| Acidiphilium organovorum | Actinomadura alba | Agromyces ramosus | Aneurinibacillus | Azohydromonas lata |
| Acidiphilium rubrum | Actinomadura atramentaria | Agromyces rhizospherae | thermoaerophilus | Azomonas |
| Acidisoma | Actinomadura | Akkermansia | Angiococcus | Azomonas agilis |
| Acidisoma sibiricum | bangladeshensis | Akkermansia muciniphila | Angiococcus disciformis | Azomonas insignis |
| Acidisoma tundrae | Actinomadura catellatispora | Albidiferax | Angulomicrobium | Azomonas macrocytogenes |
| Acidisphaera | Actinomadura chibensis | Albidiferax ferrireducens | Angulomicrobium tetraedrale | Azorhizobium |
| Acidisphaera rubrifaciens | Actinomadura chokoriensis | Albidovulum | Anoxybacillus | Azorhizobium caulinodans |
| Acidithiobacillus | Actinomadura citrea | Albidovulum inexpectatum | Anoxybacillus pushchinoensis | Azorhizophilus |
| Acidithiobacillus albertensis | Actinomadura coerulea | Alcaligenes | Aquabacterium | Azorhizophilus paspali |
| Acidithiobacillus caldus | Actinomadura echinospora | Alcaligenes denitrificans | Aquabacterium commune | Azospirillum |
| Acidithiobacillus ferrooxidans | Actinomadura fibrosa | Alcaligenes faecalis | Aquabacterium parvum | Azospirillum brasilense |
| Acidithiobacillus thiooxidans | Actinomadura formosensis | Alcanivorax | | Azospirillum halopraeferens |
| Acidobacterium | Actinomadura hibisca | Alcanivorax borkumensis | | Azospirillum irakense |
| Acidobacterium capsulatum | Actinomadura kijaniata | Alcanivorax jadensis | | Azotobacter |
| | Actinomadura latina | Algicola | | Azotobacter beijerinckii |
| | Actinomadura livida | Algicola bacteriolytica | | Azotobacter chroococcum |
| | Actinomadura | Alicyclobacillus | | Azotobacter nigricans |
| | luteofluorescens | Alicyclobacillus | | Azotobacter salinestris |
| | Actinomadura macra | disulfidooxidans | | Azotobacter vinelandii |
| | Actinomadura madurae | Alicyclobacillus | | |
| | Actinomadura oligospora | sendaiensis | | |
| | Actinomadura pelletieri | Alicyclobacillus vulcanalis | | |
| | Actinomadura rubrobrunea | Alishewanella | | |
| | Actinomadura rugatobispora | Alishewanella fetalis | | |
| | Actinomadura umbrina | | | |
| | Actinomadura | Alkalibacillus | | |
| | verrucosospora | Alkalibacillus | | |
| | Actinomadura vinacea | haloalkaliphilus | | |
| | Actinomadura viridilutea | | | |
| | Actinomadura viridis | | | |
| | Actinomadura yumaensis | | | |
| Bacillus | Bacteroides | Bibersteinia | Borrelia | Brevinema |
| [see below] | Bacteroides caccae | Bibersteinia trehalosi | Borrelia afzelii | Brevinema andersonii |
| | Bacteroides coagulans | Bifidobacterium | Borrelia americana | Brevundimonas |
| Bacteriovorax | Bacteroides eggerthii | Bifidobacterium adolescentis | Borrelia burgdorferi | Brevundimonas alba |
| Bacteriovorax stolpii | Bacteroides fragilis | Bifidobacterium angulatum | Borrelia carolinensis | Brevundimonas aurantiaca |
| | Bacteroides galacturonicus | Bifidobacterium animalis | Borrelia coriaceae | Brevundimonas diminuta |
| | Bacteroides helcogenes | Bifidobacterium asteroides | Borrelia garinii | Brevundimonas intermedia |
| | Bacteroides ovatus | Bifidobacterium bifidum | Borrelia japonica | Brevundimonas subvibrioides |
| | Bacteroides pectinophilus | Bifidobacterium boum | Bosea | Brevundimonas vancanneytii |
| | Bacteroides pyogenes | Bifidobacterium breve | Bosea minatitlanensis | Brevundimonas variabilis |
| | Bacteroides salyersiae | Bifidobacterium catenulatum | Bosea thiooxidans | Brevundimonas vesicularis |
| | Bacteroides stercoris | Bifidobacterium choerinum | Brachybacterium | Brochothrix |
| | Bacteroides suis | Bifidobacterium coryneforme | Brachybacterium | Brochothrix campestris |
| | Bacteroides tectus | Bifidobacterium cuniculi | alimentarium | Brochothrix thermosphacta |
| | Bacteroides thetaiotaomicron | Bifidobacterium dentium | Brachybacterium faecium | |
| | Bacteroides uniformis | Bifidobacterium gallicum | Brachybacterium | Brucella |
| | Bacteroides ureolyticus | Bifidobacterium gallinarum | paraconglomeratum | Brucella canis |
| | Bacteroides vulgatus | Bifidobacterium indicum | Brachybacterium rhamnosum | Brucella neotomae |
| | Balnearium | Bifidobacterium longum | Brachybacterium | Bryobacter |
| | Balnearium lithotrophicum | Bifidobacterium | tyrofermentans | Bryobacter aggregatus |
| | Balneatrix | magnumBifidobacterium | Brachyspira | Burkholderia |
| | Balneatrix alpica | merycicum | Brachyspira alvinipulli | Burkholderia ambifaria |
| | Balneola | Bifidobacterium minimum | Brachyspira hyodysenteriae | Burkholderia andropogonis |
| | Balneola vulgaris | Bifidobacterium | Brachyspira innocens | Burkholderia anthina |
| | Barnesiella | pseudocatenulatum | Brachyspira murdochii | Burkholderia caledonica |
| | Barnesiella viscericola | Bifidobacterium | Brachyspira pilosicoli | Burkholderia caryophylli |
| | Bartonella | pseudolongum | | Burkholderia cenocepacia |
| | Bartonella alsatica | Bifidobacterium pullorum | Bradyrhizobium | Burkholderia cepacia |
| | Bartonella bacilliformis | Bifidobacterium ruminantium | Bradyrhizobium canariense | Burkholderia cocovenenans |
| | Bartonella clarridgeiae | Bifidobacterium saeculare | Bradyrhizobium elkanii | Burkholderia dolosa |
| | Bartonella doshiae | Bifidobacterium subtile | Bradyrhizobium japonicum | Burkholderia fungorum |
| | Bartonella elizabethae | Bifidobacterium | Bradyrhizobium liaoningense | Burkholderia glathei |
| | Bartonella grahamii | thermophilum | Brenneria | Burkholderia glumae |
| | Bartonella henselae | Bilophila | Brenneria alni | Burkholderia graminis |
| | Bartonella rochalimae | Bilophila wadsworthia | Brenneria nigrifluens | Burkholderia kururiensis |
| | Bartonella vinsonii | Biostraticola | Brenneria quercina | Burkholderia multivorans |
| | Bavariicoccus | Biostraticola tofi | Brenneria quercina | Burkholderia phenazinium |
| | Bavariicoccus seileri | | Brenneria salicis | Burkholderia plantarii |
| | Bdellovibrio | Bizionia | Brevibacillus | Burkholderia pyrrocinia |
| | Bdellovibrio bacteriovorus | Bizionia argentinensis | Brevibacillus agri | Burkholderia silvatlantica |
| | Bdellovibrio exovorus | Blastobacter | Brevibacillus borstelensis | Burkholderia stabilis |
| | Beggiatoa | Blastobacter capsulatus | Brevibacillus brevis | Burkholderia thailandensis |
| | Beggiatoa alba | Blastobacter denitrificans | Brevibacillus centrosporus | Burkholderia tropica |
| | Beijerinckia | Blastococcus | Brevibacillus choshinensis | Burkholderia unamae |
| | Beijerinckia derxii | Blastococcus aggregatus | Brevibacillus invocatus | Burkholderia vietnamiensis |
| | Beijerinckia fluminensis | Blastococcus saxobsidens | Brevibacillus laterosporus | Buttiauxella |
| | Beijerinckia indica | Blastochloris | Brevibacillus parabrevis | Buttiauxella agrestis |
| | Beijerinckia mobilis | Blastochloris viridis | Brevibacillus reuszeri | Buttiauxella brennerae |
| | Belliella | Blastomonas | Brevibacterium | Buttiauxella ferragutiae |
| | Belliella baltica | Blastomonas natatoria | Brevibacterium abidum | Buttiauxella gaviniae |
| | Bellilinea | Blastopirellula | Brevibacterium album | Buttiauxella izardii |
| | Bellilinea caldifistulae | Blastopirellula marina | Brevibacterium aurantiacum | Buttiauxella noackiae |
| | Belnapia | Blautia | Brevibacterium celere | Buttiauxella warmboldiae |
| | Belnapia moabensis | Blautia coccoides | Brevibacterium epidermidis | Butyrivibrio |
| | Bergeriella | Blautia hansenii | Brevibacterium | Butyrivibrio fibrisolvens |
| | Bergeriella denitrificans | Blautia producta | frigoritolerans | Butyrivibrio hungatei |
| | Beutenbergia | Blautia wexlerae | Brevibacterium halotolerans | Butyrivibrio proteoclasticus |
| | Beutenbergia cavernae | Bogoriella | Brevibacterium iodinum | |
| | | Bogoriella caseilytica | Brevibacterium linens | |
| | | Bordetella | Brevibacterium lyticum | |
| | | Bordetella avium | Brevibacterium mcbrellneri | |
| | | Bordetella bronchiseptica | Brevibacterium otitidis | |
| | | Bordetella hinzii | Brevibacterium oxydans | |
| | | Bordetella holmesii | Brevibacterium paucivorans | |
| | | Bordetella parapertussis | Brevibacterium stationis | |
| | | Bordetella pertussis | | |
| | | Bordetella petrii | | |
| | | Bordetella trematum | | |
| Bacillus | | | | |
| B. acidiceler | B. aminovorans | B. glucanolyticus | B. taeanensis | B. lautus |
| B. acidicola | B. amylolyticus | B. gordonae | B. tequilensis | B. lehensis |
| B. acidiproducens | B. andreesenii | B. gottheilii | B. thermantarcticus | B. lentimorbus |
| B. acidocaldarius | B. aneurinilyticus | B. graminis | B. thermoaerophilus | B. lentus |
| B. acidoterrestris | B. anthracis | B. halmapalus | B. thermoamylovorans | B. licheniformis |
| B. aeolius | B. aquimaris | B. haloalkaliphilus | B. thermocatenulatus | B. ligniniphilus |
| B. aerius | B. arenosi | B. halochares | B. thermocloacae | B. litoralis |
| B. aerophilus | B. arseniciselenatis | B. halodenitrificans | B. thermocopriae | B. locisalis |
| B. agaradhaerens | B. arsenicus | B. halodurans | B. thermodenitrificans | B. luciferensis |
| B. agri | B. aurantiacus | B. halophilus | B. thermoglucosidasius | B. luteolus |
| B. aidingensis | B. arvi | B. halosaccharovorans | B. thermolactis | B. luteus |
| B. akibai | B. aryabhattai | B. hemicellulosilyticus | B. thermoleovorans | B. macauensis |
| B. alcalophilus | B. asahii | B. hemicentroti | B. thermophilus | B. macerans |
| B. algicola | B. atrophaeus | B. herbersteinensis | B. thermoruber | B. macquariensis |
| B. alginolyticus | B. axarquiensis | B. horikoshii | B. thermosphaericus | B. macyae |
| B. alkalidiazotrophicus | B. azotofixans | B. horneckiae | B. thiaminolyticus | B. malacitensis |
| B. alkalinitrilicus | B. azotoformans | B. horti | B. thioparans | B. mannanilyticus |
| B. alkalisediminis | B. badius | B. huizhouensis | B. thuringiensis | B. marisflavi |
| B. alkalitelluris | B. barbaricus | B. humi | B. tianshenii | B. marismortui |
| B. altitudinis | B. bataviensis | B. hwajinpoensis | B. trypoxylicola | B. marmarensis |
| B. alveayuensis | B. beijingensis | B. idriensis | B. tusciae | B. massiliensis |
| B. alvei | B. benzoevorans | B. indicus | B. validus | B. megaterium |
| B. amyloliquefaciens | B. beringensis | B. infantis | B. vallismortis | B. mesonae |
| B. | B. berkeleyi | B. infernus | B. vedderi | B. methanolicus |
| a. subsp. amyloliquefaciens | B. beveridgei | B. insolitus | B. velezensis | B. methylotrophicus |
| B. a. subsp. plantarum | B. bogoriensis | B. invictae | B. vietnamensis | B. migulanus |
| | B. boroniphilus | B. iranensis | B. vireti | B. mojavensis |
| B. dipsosauri | B. borstelensis | B. isabeliae | B. vulcani | B. mucilaginosus |
| B. drentensis | B. brevis Migula | B. isronensis | B. wakoensis | B. muralis |
| B. edaphicus | B. butanolivorans | B. jeotgali | B. weihenstephanensis | B. murimartini |
| B. ehimensis | B. canaveralius | B. kaustophilus | B. xiamenensis | B. mycoides |
| B. eiseniae | B. carboniphilus | B. kobensis | B. xiaoxiensis | B. naganoensis |
| B. enclensis | B. cecembensis | B. kochii | B. zhanjiangensis | B. nanhaiensis |
| B. endophyticus | B. cellulosilyticus | B. kokeshiiformis | B. peoriae | B. nanhaiisediminis |
| B. endoradicis | B. centrosporus | B. koreensis | B. persepolensis | B. nealsonii |
| B. farraginis | B. cereus | B. korlensis | B. persicus | B. neidei |
| B. fastidiosus | B. chagannorensis | B. kribbensis | B. pervagus | B. neizhouensis |
| B. fengqiuensis | B. chitinolyticus | B. krulwichiae | B. plakortidis | B. niabensis |
| B. firmus | B. chondroitinus | B. laevolacticus | B. pocheonensis | B. niacini |
| B. flexus | B. choshinensis | B. larvae | B. polygoni | B. novalis |
| B. foraminis | B. chungangensis | B. laterosporus | B. polymyxa | B. oceanisediminis |
| B. fordii | B. cibi | B. salexigens | B. popilliae | B. odysseyi |
| B. formosus | B. circulans | B. saliphilus | B. pseudalcalophilus | B. okhensis |
| B. fortis | B. clarkii | B. schlegelii | B. pseudofirmus | B. okuhidensis |
| B. fumarioli | B. clausii | B. sediminis | B. pseudomycoides | B. oleronius |
| B. funiculus | B. coagulans | B. selenatarsenatis | B. psychrodurans | B. oryzaecorticis |
| B. fusiformis | B. coahuilensis | B. selenitireducens | B. psychrophilus | B. oshimensis |
| B. galactophilus | B. cohnii | B. seohaeanensis | B. psychrosaccharolyticus | B. pabuli |
| B. galactosidilyticus | B. composti | B. shacheensis | B. psychrotolerans | B. pakistanensis |
| B. galliciensis | B. curdlanolyticus | B. shackletonii | B. pulvifaciens | B. pallidus |
| B. gelatini | B. cycloheptanicus | B. siamensis | B. pumilus | B. pallidus |
| B. gibsonii | B. cytotoxicus | B. silvestris | B. purgationiresistens | B. panacisoli |
| B. ginsengi | B. daliensis | B. simplex | B. pycnus | B. panaciterrae |
| B. ginsengihumi | B. decisifrondis | B. siralis | B. qingdaonensis | B. pantothenticus |
| B. ginsengisoli | B. decolorationis | B. smithii | B. qingshengii | B. parabrevis |
| B. globisporus (eg, B. | B. deserti | B. soli | B. reuszeri | B. paraflexus |
| g. subsp. Globisporus; or B. | | B. solimangrovi | B. rhizosphaerae | B. pasteurii |
| g. subsp. Marinus) | | B. solisalsi | B. rigui | B. patagoniensis |
| | | B. songklensis | B. ruris | |
| | | B. sonorensis | B. safensis | |
| | | B. sphaericus | B. salarius | |
| | | B. sporothermodurans | | |
| | | B. stearothermophilus | | |
| | | B. stratosphericus | | |
| | | B. subterraneus | | |
| | | B. subtilis (eg, B. | | |
| | | s. subsp. Inaquosorum, or B. | | |
| | | s. subsp. Spizizenr, or B. | | |
| | | s. subsp. Subtilis) | | |
| Caenimonas | Campylobacter | Cardiobacterium | Catenuloplanes | Curtobacterium |
| Caenimonas koreensis | Campylobacter coli | Cardiobacterium hominis | Catenuloplanes atrovinosus | Curtobacterium |
| Caldalkalibacillus | Campylobacter concisus | Carnimonas | Catenuloplanes castaneus | albidum |
| Caldalkalibacillus uzonensis | Campylobacter curvus | Carnimonas nigrificans | Catenuloplanes crispus | Curtobacterium citreus |
| Caldanaerobacter | Campylobacter fetus | Carnobacterium | Catenuloplanes indicus | |
| Caldanaerobacter subterraneus | Campylobacter gracilis | Carnobacterium | Catenuloplanes japonicus | |
| Caldanaerobius | Campylobacter helveticus | alterfunditum | Catenuloplanes nepalensis | |
| Caldanaerobius fijiensis | Campylobacter hominis | Carnobacterium divergens | Catenuloplanes niger | |
| Caldanaerobius | Campylobacter hyointestinalis | Carnobacterium funditum | Chryseobacterium | |
| polysaccharolyticus | Campylobacter jejuni | Carnobacterium gallinarum | Chryseobacterium | |
| Caldanaerobius zeae | Campylobacter lari | Carnobacterium | balustinum | |
| | Campylobacter mucosalis | maltaromaticum | | |
| Caldanaerovirga | Campylobacter rectus | Carnobacterium mobile | Citrobacter | |
| Caldanaerovirga acetigignens | Campylobacter showae | Carnobacterium viridans | C. amalonaticus | |
| Caldicellulosiruptor | Campylobacter sputorum | Caryophanon | C. braakii | |
| Caldicellulosiruptor bescii | Campylobacter upsaliensis | Caryophanon latum | C. diversus | |
| Caldicellulosiruptor kristjanssonii | Capnocytophaga | Caryophanon tenue | C. farmeri | |
| Caldicellulosiruptor owensensis | Capnocytophaga canimorsus | Catellatospora | C. freundii | |
| | Capnocytophaga cynodegmi | Catellatospora citrea | C. gillenii | |
| | Capnocytophaga gingivalis | Catellatospora | C. koseri | |
| | Capnocytophaga granulosa | methionotrophica | C. murliniae | |
| | Capnocytophaga haemolytica | Catenococcus | C. pasteurii^{[1]} | |
| | Capnocytophaga ochracea | Catenococcus thiocycli | C. rodentium | |
| | Capnocytophaga sputigena | | C. sedlakii | |
| | | | C. werkmanii | |
| | | | C. youngae | |
| | | | Clostridium | |
| | | | (see below) | |
| | | | Coccochloris | |
| | | | Coccochloris elabens | |
| | | | Corynebacterium | |
| | | | Corynebacterium flavescens | |
| | | | Corynebacterium variabile | |
| Clostridium | | | | |

Clostridium absonum, Clostridium aceticum, Clostridium acetireducens, Clostridium acetobutylicum, Clostridium acidisoli, Clostridium aciditolerans, Clostridium acidurici, Clostridium aerotolerans, Clostridium
aestuarii, Clostridium akagii, Clostridium aldenense, Clostridium aldrichii, Clostridium algidicarni, Clostridium algidixylanolyticum, Clostridium algifaecis, Clostridium algoriphilum, Clostridium alkalicellulosi,
Clostridium aminophilum, Clostridium aminovalericum, Clostridium amygdalinum, Clostridium amylolyticum, Clostridium arbusti, Clostridium arcticum, Clostridium argentinense, Clostridium asparagiforme,
Clostridium aurantibutyricum, Clostridium autoethanogenum, Clostridium baratii, Clostridium barkeri, Clostridium bartlettii, Clostridium beijerinckii, Clostridium bifermentans, Clostridium bolteae, Clostridium
bornimense, Clostridium botulinum, Clostridium bowmanii, Clostridium bryantii, Clostridium butyricum, Clostridium cadaveris, Clostridium caenicola, Clostridium caminithermale, Clostridium carboxidivorans,
Clostridium carnis, Clostridium cavendishii, Clostridium celatum, Clostridium celerecrescens, Clostridium cellobioparum, Clostridium cellulofermentans, Clostridium cellulolyticum, Clostridium cellulosi,
Clostridium cellulovorans, Clostridium chartatabidum, Clostridium chauvoei, Clostridium chromiireducens, Clostridium citroniae, Clostridium clariflavum, Clostridium clostridioforme, Clostridium coccoides,
Clostridium cochlearium, Clostridium colletant, Clostridium colicanis, Clostridium colinum, Clostridium collagenovorans, Clostridium cylindrosporum, Clostridium difficile, Clostridium diolis, Clostridium
disporicum, Clostridium drakei, Clostridium durum, Clostridium estertheticum, Clostridium estertheticum estertheticum, Clostridium estertheticum laramiense, Clostridium fallax, Clostridium felsineum, Clostridium
fervidum, Clostridium fimetarium, Clostridium formicaceticum, Clostridium frigidicarnis, Clostridium frigoris, Clostridium ganghwense, Clostridium gasigenes, Clostridium ghonii, Clostridium glycolicum,
Clostridium glycyrrhizinilyticum, Clostridium grantii, Clostridium haemolyticum, Clostridium halophilum, Clostridium hastiforme, Clostridium hathewayi, Clostridium herbivorans, Clostridium hiranonis,
Clostridium histolyticum, Clostridium homopropionicum, Clostridium huakuii, Clostridium hungatei, Clostridium hydrogeniformans, Clostridium hydroxybenzoicum, Clostridium hylemonae, Clostridium jejuense,
Clostridium indolis, Clostridium innocuum, Clostridium intestinale, Clostridium irregulare, Clostridium isatidis, Clostridium josui, Clostridium kluyveri, Clostridium lactatifermentans, Clostridium lacusfryxellense,
Clostridium laramiense, Clostridium lavalense, Clostridium lentocellum, Clostridium lentoputrescens, Clostridium leptum, Clostridium limosum, Clostridium litorale, Clostridium lituseburense, Clostridium ljungdahlii,
Clostridium lortetii, Clostridium lundense, Clostridium magnum, Clostridium malenominatum, Clostridium mangenotii, Clostridium mayombei, Clostridium methoxybenzovorans, Clostridium methylpentosum,
Clostridium neopropionicum, Clostridium nexile, Clostridium nitrophenolicum, Clostridium novyi, Clostridium oceanicum, Clostridium orbiscindens, Clostridium oroticum, Clostridium oxalicum, Clostridium
papyrosolvens, Clostridium paradoxum, Clostridium paraperfringens (Alias: C. welchii), Clostridium paraputrificum, Clostridium pascui, Clostridium pasteurianum, Clostridium peptidivorans, Clostridium perenne,
Clostridium perfringens, Clostridium pfennigii, Clostridium phytofermentans, Clostridium piliforme, Clostridium polysaccharolyticum, Clostridium populeti, Clostridium propionicum, Clostridium proteoclasticum,
Clostridium proteolyticum, Clostridium psychrophilum, Clostridium puniceum, Clostridium purinilyticum, Clostridium putrefaciens, Clostridium putrificum, Clostridium quercicolum, Clostridium quinii,
Clostridium ramosum, Clostridium rectum, Clostridium roseum, Clostridium saccharobutylicum, Clostridium saccharogumia, Clostridium saccharolyticum, Clostridium saccharoperbutylacetonicum, Clostridium
sardiniense, Clostridium sartagoforme, Clostridium scatologenes, Clostridium schirmacherense, Clostridium scindens, Clostridium septicum, Clostridium sordellii, Clostridium sphenoides, Clostridium spiroforme,
Clostridium sporogenes, Clostridium sporosphaeroides, Clostridium stercorarium, Clostridium stercorarium leptospartum, Clostridium stercorarium stercorarium, Clostridium stercorarium thermolacticum,
Clostridium sticklandii, Clostridium straminisolvens, Clostridium subterminale, Clostridium sufflavum, Clostridium sulfidigenes, Clostridium symbiosum, Clostridium tagluense, Clostridium
tepidiprofundi, Clostridium termitidis, Clostridium tertium, Clostridium tetani, Clostridium tetanomorphum, Clostridium thermaceticum, Clostridium thermautotrophicum, Clostridium thermoalcaliphilum,
Clostridium thermobutyricum, Clostridium thermocellum, Clostridium thermocopriae, Clostridium thermohydrosulfuricum, Clostridium thermolacticum, Clostridium thermopalmarium,
Clostridium thermopapyrolyticum, Clostridium thermosaccharolyticum, Clostridium thermosuccinogenes, Clostridium thermosulfurigenes, Clostridium thiosulfatireducens, Clostridium tyrobutyricum,
Clostridium uliginosum, Clostridium ultunense, Clostridium villosum, Clostridium vincentii, Clostridium viride, Clostridium xylanolyticum, Clostridium xylanovorans

| | | | | |
|---|---|---|---|---|
| Dactylosporangium | Deinococcus | Delftia | Echinicola | |
| Dactylosporangium aurantiacum | Deinococcus aerius | Delftia acidovorans | Echinicola pacifica | |
| Dactylosporangium fulvum | Deinococcus apachensis | Desulfovibrio | Echinicola vietnamensis | |
| Dactylosporangium matsuzakiense | Deinococcus aquaticus | Desulfovibrio desulfuricans | | |
| Dactylosporangium roseum | Deinococcus aquatilis | Diplococcus | | |
| Dactylosporangium thailandense | Deinococcus caeni | Diplococcus pneumoniae | | |
| Dactylosporangium vinaceum | Deinococcus radiodurans | | | |
| | Deinococcus radiophilus | | | |
| Enterobacter | Enterobacter kobei | Faecalibacterium | Flavobacterium | |
| E. aerogenes | E. ludwigii | Faecalibacterium prausnitzii | Flavobacterium antarcticum | |
| E. amnigemis | E. mori | Fangia | Flavobacterium aquatile | |
| E. agglomerans | E. nimipressuralis | Fangia hongkongensis | Flavobacterium | |
| E. arachidis | E. oryzae | Fastidiosipila | aquidurense | |
| E. asburiae | E. pulveris | Fastidiosipila sanguinis | Flavobacterium balustinum | |
| E. cancerogenous | E. pyrinus | Fusobacterium | Flavobacterium croceum | |
| E. cloacae | E. radicincitans | Fusobacterium nucleatum | Flavobacterium cucumis | |
| E. cowanii | E. taylorae | | Flavobacterium | |
| E. dissolvens | E. turicensis | | daejeonense | |
| E. gergoviae | E. sakazakii Enterobacter soli | | Flavobacterium defluvii | |
| E. helveticus | Enterococcus | | Flavobacterium degerlachei | |
| E. hormaechei | Enterococcus durans | | Flavobacterium | |
| E. intermedins | Enterococcus faecalis | | denitrificans | |
| | Enterococcus faecium | | Flavobacterium filum | |
| | Erwinia | | Flavobacterium flevense | |
| | Erwinia hapontici | | Flavobacterium frigidarium | |
| | Escherichia | | Flavobacterium mizutaii | |
| | Escherichia coli | | Flavobacterium | |
| | | | okeanokoites | |
| Gaetbulibacter | Haemophilus | Ideonella | Janibacter | |
| Gaetbulibacter saemankumensis | Elaemophilus aegyptius | Ideonella azotifigens | Janibacter anophelis | |
| Gallibacterium | Elaemophilus aphrophilus | Idiomarina | Janibacter corallicola | |
| Gallibacterium anatis | Haemophilus felis | Idiomarina abyssalis | Janibacter limosus | |
| Gallicola | Haemophilus gallinarum | Idiomarina baltica | Janibacter melonis | |
| Gallicola barnesae | Haemophilus haemolyticus | Idiomarina fontislapidosi | Janibacter terrae | |
| Garciella | Haemophilus influenzae | Idiomarina loihiensis | Jannaschia | |
| Garciella nitratireducens | Haemophilus paracuniculus | Idiomarina ramblicola | Jannaschia cystaugens | |
| Geobacillus | Haemophilus parahaemolyticus | Idiomarina seosinensis | Jannaschia helgolandensis | |
| Geobacillus thermoglucosidasius | Haemophilus parainfluenzae | Idiomarina zobellii | Jannaschia pohangensis | |
| Geobacillus stearothermophilus | Haemophilus | Ignatzschineria | Jannaschia rubra | |
| Geobacter | paraphrohaemolyticus | Ignatzschineria larvae | | |
| Geobacter bemidjiensis | Haemophilus parasuis | | Janthinobacterium | |
| Geobacter bremensis | Haemophilus pittmaniae | Ignavigranum | Janthinobacterium | |
| Geobacter chapellei | Hafnia | Ignavigranum ruoffiae | agaricidamnosum | |
| Geobacter grbiciae | Hafnia alvei | Ilumatobacter | Janthinobacterium lividum | |
| Geobacter hydrogenophilus | Hahella | Ilumatobacter fluminis | Jejuia | |
| Geobacter lovleyi | Hahella ganghwensis | Ilyobacter | Jejuia pallidilutea | |
| Geobacter metallireducens | Halalkalibacillus | Ilyobacter delafieldii | Jeotgalibacillus | |
| Geobacter pelophilus | Halalkalibacillus halophilus | Ilyobacter insuetus | Jeotgalibacillus | |
| Geobacter pickeringii | Helicobacter | Ilyobacter polytropus | alimentarius | |
| Geobacter sulfurreducens | Helicobacter pylori | Ilyobacter tartaricus | Jeotgalicoccus | |
| | | | Jeotgalicoccus halotolerans | |
| Geodermatophilus | | | | |
| Geodermatophilus obscurus | | | | |
| Gluconacetobacter | | | | |
| Gluconacetobacter xylinus | | | | |
| Gordonia | | | | |
| Gordonia rubripertincta | | | | |
| Kaistia | Labedella | Listeria ivanovii | Micrococcus | Nesterenkonia |
| Kaistia adipata | Labedella gwakjiensis | L. marthii | Micrococcus luteus | Nesterenkonia holobia |
| Kaistia soli | Labrenzia | L. monocytogenes | Micrococcus lylae | Nocardia |
| Kangiella | Labrenzia aggregata | L. newyorkensis | Moraxella | Nocardia argentinensis |
| Kangiella aquimarina | Labrenzia alba | L. riparia | Moraxella bovis | Nocardia corallina |
| Kangiella koreensis | Labrenzia alexandrii | L. rocourtiae | Moraxella nonliquefaciens | Nocardia |
| | Labrenzia marina | L. seeligeri | Moraxella osloensis | otitidiscaviarum |
| Kerstersia | Labrys | L. weihenstephanensis | Nakamurella | |
| Kerstersia gyiorum | Labrys methylaminiphilus | L. welshimeri | Nakamurella multipartita | |
| Kiloniella | Labrys miyagiensis | Listonella | Nannocystis | |
| Kiloniella laminariae | Labrys monachus | Listonella anguillarum | Nannocystis pusilia | |
| Klebsiella | Labrys okinawensis | Macrococcus | Natranaerobius | |
| K. gramilomatis | Labrys portucalensis | Macrococcus bovicus | Natranaerobius | |
| K. oxytoca | | Marinobacter | thermophilus | |
| K. pneumoniae | Lactobacillus | Marinobacter algicola | Natranaerobius trueperi | |
| K. terrigena | [see below] | Marinobacter bryozoorum | Naxibacter | |
| K. variicola | Laceyella | Marinobacter flavimaris | Naxibacter alkalitolerans | |
| Kluyvera | Laceyella putida | Meiothermus | Neisseria | |
| Kluyvera ascorbata | Lechevalieria | Meiothermus ruber | Neisseria cinerea | |
| Kocuria | Lechevalieria aerocolonigenes | Methylophilus | Neisseria denitrificans | |
| Kocuria roasea | Legionella | Methylophilus | Neisseria gonorrhoeae | |
| Kocuria varians | [see below] | methylotrophus | Neisseria lactamica | |
| Kurthia | Listeria | Microbacterium | Neisseria mucosa | |
| Kurthia zopfii | L. aquatica | Microbacterium | Neisseria sicca | |
| | L. booriae | ammoniaphilum | Neisseria subflava | |
| | L. cornellensis | Microbacterium arborescens | Neptunomonas | |
| | L. fleischmannii | Microbacterium liquefaciens | Neptunomonas japonica | |
| | L. floridensis | Microbacterium oxydans | | |
| | L. grandensis | | | |
| | L. grayi | | | |
| | L. innocua | | | |
| Lactobacillus | | | | |
| L. acetotolerans | L. catenaformis | L. mali | L. parakefiri | L. sakei |
| L. acidifarinae | L. ceti | L. manihotivorans | L. paralimentarius | L. salivarius |
| L. acidipiscis | L. coleohominis | L. mindensis | L. paraplantarum | L. sanfranciscensis |
| L. acidophilus | L. collinoides | L. mucosae | L. pentosus | L. satsumensis |
| Lactobacillus agilis | L. composti | L. murinus | L. perolens | L. secaliphilus |
| L. algidus | L. concavus | L. nagelii | L. plantarum | L. sharpeae |
| L. alimentarius | L. coryniformis | L. namurensis | L. pontis | L. siliginis |
| L. amylolyticus | L. crispatus | L. nantensis | L. protectus | L. spicheri |
| L. amylophilus | L. crustorum | L. oligofermentans | L. psittaci | L. suebicus |
| L. amylotrophicus | L. curvatus | L. oris | L. rennini | L. thailandensis |
| L. amylovorus | L. delbrueckii subsp. | L. panis | L. reuteri | L. ultunensis |
| L. animalis | bulgaricus | L. pantheris | L. rhamnosus | L. vaccinostercus |
| L. antri | L. delbrueckii subsp. | L. parabrevis | L. rimae | L. vaginalis |
| L. apodemi | delbrueckii | L. parabuchneri | L. rogosae | L. versmoldensis |
| L. aviarius | L. delbrueckii subsp. lactis | L. paracasei | L. rossiae | L. vini |
| L. bifermentans | L. dextrinicus | L. paracollinoides | L. ruminis | L. vitulinus |
| L. brevis | L. diolivorans | L. parafarraginis | L. saerimneri | L. zeae |
| L. buchneri | L. equi | L. homohiochii | L. jensenii | L. zymae |
| L. camelliae | L. equigenerosi | L. iners | L. johnsonii | L. gastricus |
| L. casei | L. farraginis | L. ingluviei | L. kalixensis | L. ghanensis |
| L. kitasatonis | L. farciminis | L. intestinalis | L. kefiranofaciens | L. graminis |
| L. kunkeei | L. fermentum | L. fuchuensis | L. kefiri | L. hammesii |
| L. leichmannii | L. fornicalis | L. gallinarum | L. kimchii | L. hamsteri |
| L. lindneri | L. fructivorans | L. gasseri | L. helveticus | L. harbinensis |
| L. malefermentans | L. frumenti | | L. hilgardii | L. hayakitensis |
| Legionella | | | | |
| Legionella adelaidensis | Legionella drancourtii | Candidatus Legionella jeonii | Legionella quinlivanii | |
| Legionella anisa | Legionella dresdenensis | Legionella jordanis | Legionella rowbothamii | |
| Legionella beliardensis | Legionella drozanskii | Legionella lansingensis | Legionella rubrilucens | |
| Legionella birminghamensis | Legionella dumoffii | Legionella londiniensis | Legionella sainthelensi | |
| Legionella bozemanae | Legionella erythra | Legionella longbeachae | Legionella santicrucis | |
| Legionella brunensis | Legionella fairfieldensis | Legionella lytica | Legionella shakespearei | |
| Legionella busanensis | Legionella fallonii | Legionella maceachernii | Legionella spiritensis | |
| Legionella cardiaca | Legionella feeleii | Legionella massiliensis | Legionella steelei | |
| Legionella cherrii | Legionella geestiana | Legionella micdadei | Legionella steigerwaltii | |
| Legionella cincinnatiensis | Legionella genomospecies | Legionella monrovica | Legionella taurinensis | |
| Legionella clemsonensis | Legionella gormanii | Legionella moravica | Legionella tucsonensis | |
| Legionella donaldsonii | Legionella gratiana | Legionella nagasakiensis | Legionella tunisiensis | |
| | Legionella gresilensis | Legionella nautarum | Legionella wadsworthii | |
| | Legionella hackeliae | Legionella norrlandica | Legionella waltersii | |
| | Legionella impletisoli | Legionella oakridgensis | Legionella worsleiensis | |
| | Legionella israelensis | Legionella parisiensis | Legionella yabuuchiae | |
| | Legionella jamestowniensis | Legionella pittsburghensis | | |
| | | Legionella pneumophila | | |
| | | Legionella quateirensis | | |
| Oceanibulbus | Paenibacillus | Prevotella | Quadrisphaera | |
| Oceanibulbus indolifex | Paenibacillus thiaminolyticus | Prevotella albensis | Quadrisphaera granulorum | |
| Oceanicaulis | Pantoea | Prevotella amnii | Quatrionicoccus | |
| Oceanicaulis alexandrii | Pantoea agglomerans | Prevotella bergensis | Quatrionicoccus | |
| Oceanicola | | Prevotella bivia | australiensis | |
| Oceanicola batsensis | Paracoccus | Prevotella brevis | | |
| Oceanicola granulosus | Paracoccus alcaliphilus | Prevotella bryantii | Quinella | |
| Oceanicola nanhaiensis | Paucimonas | Prevotella buccae | Quinella ovalis | |
| Oceanimonas | Paucimonas lemoignei | Prevotella buccalis | | |
| Oceanimonas baumannii | Pectobacterium | Prevotella copri | Ralstonia | |
| Oceaniserpentilla | Pectobacterium aroidearum | Prevotella dentalis | Ralstonia eutropha | |
| Oceaniserpentilla haliotis | Pectobacterium atrosepticum | Prevotella denticola | Ralstonia insidiosa | |
| Oceanisphaera | Pectobacterium | Prevotella disiens | Ralstonia mannitolilytica | |
| Oceanisphaera donghaensis | betavasculorum | Prevotella histicola | Ralstonia pickettii | |
| Oceanisphaera litoralis | Pectobacterium cacticida | Prevotella intermedia | Ralstonia | |
| Oceanithermus | Pectobacterium carnegieana | Prevotella maculosa | pseudosolanacearum | |
| Oceanithermus desulfurans | Pectobacterium carotovorum | Prevotella marshii | Ralstonia syzygii | |
| Oceanithermus profundus | Pectobacterium chrysanthemi | Prevotella melaninogenica | Ralstonia solanacearum | |
| Oceanobacillus | Pectobacterium cypripedii | Prevotella micans | Ramlibacter | |
| Oceanobacillus caeni | Pectobacterium rhapontici | Prevotella multiformis | Ramlibacter henchirensis | |
| Oceanospirillum | Pectobacterium wasabiae | Prevotella nigrescens | Ramlibacter tataouinensis | |
| Oceanospirillum linum | Pianococcus | Prevotella oralis | | |
| | Pianococcus citreus | Prevotella oris | | |
| | Planomicrobium | Prevotella oulorum | Raoultella | |
| | Planomicrobium okeanokoites | Prevotella pallens | Raoultella ornithinolytica | |
| | Plesiomonas | Prevotella salivae | Raoultella planticola | |
| | Plesiomonas shigelloides | Prevotella stercorea | Raoultella terrigena | |
| | Proteus | Prevotella tannerae | Rathayibacter | |
| | Proteus vulgaris | Prevotella timonensis | Rathayibacter caricis | |
| | | Prevotella veroralis | Rathayibacter festucae | |
| | | Providencia | Rathayibacter iranicus | |
| | | Providencia stuartii | Rathayibacter rathayi | |
| | | Pseudomonas | Rathayibacter toxicus | |
| | | Pseudomonas aeruginosa | Rathayibacter tritici | |
| | | Pseudomonas alcaligenes | Rhodobacter | |
| | | Pseudomonas anguillispetica | Rhodobacter sphaeroides | |
| | | Pseudomonas fluorescens | Ruegeria | |
| | | Pseudoalteromonas | Ruegeria gelatinovorans | |
| | | haloplanktis | | |
| | | Pseudomonas mendocina | | |
| | | Pseudomonas | | |
| | | pseudoalcaligenes | | |
| | | Pseudomonas putida | | |
| | | Pseudomonas tutzeri | | |
| | | Pseudomonas syringae | | |
| | | Psychrobacter | | |
| | | Psychrobacter faecalis | | |
| | | Psychrobacter | | |
| | | phenylpyruvicus | | |
| Saccharococcus | Sagittula | Sanguibacter | Stenotrophomonas | Tatlockia |
| Saccharococcus thermophilus | Sagittula stellata | Sanguibacter keddieii | Stenotrophomonas | Tatlockia maceachernii |
| Saccharomonospora | Salegentibacter | Sanguibacter suarezii | maltophilia | Tatlockia micdadei |
| Saccharomonospora azurea | Salegentibacter salegens | Saprospira | Streptococcus | Tenacibaculum |
| Saccharomonospora cyanea | Salimicrobium | Saprospira grandis | | Tenacibaculum |
| Saccharomonospora viridis | Salimicrobium album | Sarcina | [also see below] | amylolyticum |
| Saccharophagus | Salinibacter | Sarcina maxima | Streptomyces | Tenacibaculum discolor |
| Saccharophagus degradans | Salinibacter ruber | Sarcina ventriculi | Streptomyces | Tenacibaculum |
| Saccharopolyspora | Salinicoccus | Sebaldella | achromogenes | gallaicum |
| Saccharopolyspora erythraea | Salinicoccus alkaliphilus | Sebaldella termitidis | Streptomyces cesalbus | Tenacibaculum |
| Saccharopolyspora gregorii | Salinicoccus hispanicus | | Streptomyces cescaepitosus | lutimaris |
| Saccharopolyspora hirsuta | Salinicoccus roseus | Serratia | Streptomyces cesdiastaticus | Tenacibaculum |
| Saccharopolyspora hordei | Salinispora | Serratia fonticola | Streptomyces cesexfoliatus | mesophilum |
| Saccharopolyspora rectivirgula | Salinispora arenicola | Serratia marcescens | Streptomyces fimbriatus | Tenacibaculum |
| Saccharopolyspora spinosa | Salinispora tropica | Sphaerotilus | Streptomyces fradiae | skagerrakense |
| Saccharopolyspora taberi | Salinivibrio | Sphaerotilus natans | Streptomyces fulvissimus | |
| | Salinivibrio costicola | | Streptomyces griseoruber | |
| Saccharothrix | Salmonella | Sphingobacterium | Streptomyces griseus | Tepidanaerobacter |
| Saccharothrix australiensis | Salmonella bongori | Sphingobacterium multivorum | Streptomyces lavendulae | Tepidanaerobacter |
| Saccharothrix coeruleofusca | Salmonella enterica | Staphylococcus | Streptomyces | syntrophicus |
| Saccharothrix espanaensis | Salmonella subterranea | [see below] | phaeochromogenes | Tepidibacter |
| Saccharothrix longispora | Salmonella typhi | | Streptomyces | Tepidibacter |
| Saccharothrix mutabilis | | | thermodiastaticus | formicigenes |
| Saccharothrix syringae | | | Streptomyces tubercidicus | Tepidibacter |
| Saccharothrix tangerinus | | | | thalassicus |
| Saccharothrix texasensis | | | | Thermus |
| | | | | Thermus aquaticus |
| | | | | Thermus filiformis |
| | | | | Thermus thermophilus |
| Staphylococcus | | | | |
| S. arlettae | S. equorum | S. microti | S. schleiferi | |
| S. agnetis | S. felis | S. muscae | S. sciuri | |
| S. aureus | S. fleurettii | S. nepalensis | S. simiae | |
| S. auricularis | S. gallinarum | S. pasteuri | S. simulans | |
| S. capitis | S. haemolyticus | S. petrasii | S. stepanovicii | |
| S. caprae | S. hominis | S. pettenkoferi | S. succinus | |
| S. carnosus | S. hyicus | S. piscifermentans | S. vitulinus | |
| S. caseolyticus | S. intermedius | S. pseudintermedius | S. warneri | |
| S. chromogenes | S. kloosii | S. pseudolugdunensis | S. xylosus | |
| S. cohnii | S. leei | S. pulvereri | | |
| S. condimenti | S. lentus | S. rostri | | |
| S. delphini | S. lugdunensis | S. saccharolyticus | | |
| S. devriesei | S. lutrae | S. saprophyticus | | |
| S. epidermidis | S. lyticans | | | |
| | S. massiliensis | | | |
| Streptococcus | | | | |
| Streptococcus agalactiae | Streptococcus infantarius | Streptococcus orisratti | Streptococcus thermophilus | |
| Streptococcus anginosus | Streptococcus iniae | Streptococcus parasanguinis | Streptococcus sanguinis | |
| Streptococcus bovis | Streptococcus intermedius | Streptococcus peroris | Streptococcus sobrinus | |
| Streptococcus canis | Streptococcus lactarius | Streptococcus pneumoniae | Streptococcus suis | |
| Streptococcus constellatus | Streptococcus milleri | Streptococcus | Streptococcus uberis | |
| Streptococcus downei | Streptococcus mitis | pseudopneumoniae | Streptococcus vestibularis | |
| Streptococcus dysgalactiae | Streptococcus mutans | Streptococcus pyogenes | Streptococcus viridans | |
| Streptococcus equines | Streptococcus oralis | Streptococcus ratti | Streptococcus | |
| Streptococcus faecalis | Streptococcus tigurinus | Streptococcus salivariu | zooepidemicus | |
| Streptococcus ferus | | | | |
| Uliginosibacterium | Vagococcus | Vibrio | Virgibacillus | Xanthobacter |
| | Vagococcus carniphilus | Vibrio aerogenes | Virgibacillus | Xanthobacter agilis |
| Uliginosibacterium gangwonense | Vagococcus elongatus | Vibrio aestuarianus | halodenitrificans | Xanthobacter |
| Ulvibacter | Vagococcus fessus | Vibrio albensis | Virgibacillus | aminoxidans |
| Ulvibacter litoralis | Vagococcus fluvialis | Vibrio alginolyticus | pantothenticus | Xanthobacter |
| Umezawaea | Vagococcus lutrae | Vibrio campbellii | Weissella | autotrophicus |
| Umezawaea tangerina | Vagococcus salmoninarum | Vibrio cholerae | Weissella cibaria | Xanthobacter flavus |
| Undibacterium | Variovorax | Vibrio cincinnatiensis | Weissella confusa | Xanthobacter tagetidis |
| Undibacterium pigrum | Variovorax boronicumulans | Vibrio coralliilyticus | Weissella halotolerans | Xanthobacter viscosus |
| Ureaplasma | Variovorax dokdonensis | Vibrio cyclitrophicus | Weissella hellenica | Xanthomonas |
| Ureaplasma urealyticum | Variovorax paradoxus | Vibrio diazotrophicus | Weissella kandleri | Xanthomonas |
| | Variovorax soli | Vibrio fluvialis | Weissella koreensis | albilineans |
| Ureibacillus | Veillonella | Vibrio furnissii | Weissella minor | Xanthomonas alfalfae |
| Ureibacillus composti | Veillonella atypica | Vibrio gazogenes | Weissella | Xanthomonas |
| Ureibacillus suwonensis | Veillonella caviae | Vibrio halioticoli | paramesenteroides | arboricola |
| Ureibacillus terrenus | Veillonella criceti | Vibrio harveyi | Weissella soli | Xanthomonas |
| Ureibacillus thermophilus | Veillonella dispar | Vibrio ichthyoenteri | Weissella thailandensis | axonopodis |
| Ureibacillus thermosphaericus | Veillonella montpellierensis | Vibrio mediterranei | Weissella viridescens | Xanthomonas |
| | Veillonella parvula | Vibrio metschnikovii | Williamsia | campestris |
| | Veillonella ratti | Vibrio mytili | Williamsia marianensis | Xanthomonas citri |
| | Veillonella rodentium | Vibrio natriegens | Williamsia maris | Xanthomonas codiaei |
| | Venenivibrio | Vibrio navarrensis | Williamsia serinedens | Xanthomonas |
| | Venenivibrio stagnispumantis | Vibrio nereis | Winogradskyella | cucurbitae |
| | | Vibrio nigripulchritudo | Winogradskyella | Xanthomonas |
| | Verminephrobacter | Vibrio ordalii | thalassocola | euvesicatoria |
| | Verminephrobacter eiseniae | Vibrio orientalis | | Xanthomonas fragariae |
| | | Vibrio parahaemolyticus | Wolbachia | Xanthomonas fuscans |
| | Verrucomicrobium | Vibrio pectenicida | Wolbachia persica | Xanthomonas gardneri |
| | Verrucomicrobium spinosum | Vibrio penaeicida | | Xanthomonas hortorum |
| | | Vibrio proteolyticus | Wolinella | Xanthomonas hyacinthi |
| | | Vibrio shilonii | Wolinella succinogenes | Xanthomonas perforans |
| | | Vibrio splendidus | | Xanthomonas phaseoli |
| | | Vibrio tubiashii | Zobellia | Xanthomonas pisi |
| | | Vibrio vulnificus | Zobellia galactanivorans | Xanthomonas populi |
| | | | Zobellia uliginosa | Xanthomonas theicola |
| | | | Zoogloea | Xanthomonas |
| | | | Zoogloea ramigera | translucens |
| | | | Zoogloea resiniphila | |
| | | | | Xanthomonas |
| | | | | vesicatoria |
| | | | | Xylella |
| | | | | Xylella fastidiosa |
| | | | | Xylophilus |
| | | | | Xylophilus ampelinus |
| Xenophilus | Yangia | Yersinia mollaretii | Zooshikella | Zobellella |
| Xenophilus azovorans | Yangia pacifica | Yersinia philomiragia | Zooshikella ganghwensis | Zobellella denitrificans |
| | | Yersinia pestis | | Zobellella taiwanensis |
| Xenorhabdus | Yaniella | Yersinia pseudotuberculosis | Zunongwangia | |
| Xenorhabdus beddingii | Yaniella flava | Yersinia rohdei | Zunongwangia profunda | Zeaxanthinibacter |
| Xenorhabdus bovienii | Yaniella halotolerans | Yersinia ruckeri | Zymobacter | Zeaxanthinibacter |
| Xenorhabdus cabanillasii | Yeosuana | Yokenella | Zymobacter palmae | enoshimensis |
| Xenorhabdus doucetiae | Yeosuana aromativorans | Yokenella regensburgei | Zymomonas | Zhihengliuella |
| Xenorhabdus griffiniae | Yersinia | Yonghaparkia | Zymomonas mobilis | Zhihengliuella |
| Xenorhabdus hominickii | Yersinia aldovae | Yonghaparkia alkaliphila | Zymophilus | halotolerans |
| Xenorhabdus koppenhoeferi | Yersinia bercovieri | Zavarzinia | Zymophilus paucivorans | Xylanibacterium |
| Xenorhabdus nematophila | Yersinia enterocolitica | Zavarzinia compransoris | Zymophilus raffinosivorans | Xylanibacterium ulmi |
| Xenorhabdus poinarii | Yersinia entomophaga | | | |
| Xylanibacter | Yersinia frederiksenii | | | |
| Xylanibacter oryzae | Yersinia intermedia | | | |
| | Yersinia kristensenii | | | |

TABLE 2
Example Cas

| Bacteria/Phage | CRISPR-Cas Type |
|---|---|
| Clostridium botulinum | Type I-B |
| Clostridium tetani | Type I-B |
| Eggerthella lenta | Type I-C |
| Moraxella bovoculi | Type I-C |
| Streptococcus mutans | Type I-C |
| Streptococcus mutans | Type I-C |
| Streptococcus pyogenes | Type I-C |
| Bacillus halodurans | Type I-C |
| Prevotella enoeca | Type I-C |
| Bacteroides fragilis | Type I-C |
| Pseudomonas aeruginosa | Type I-C |
| Nostoc sp. CENA543 | Type I-D |
| Escherichia coli | Type I-E |
| Vibrio cholerae | Type I-E |
| Citrobacter freundii | Type I-E |
| Salmonella enterica | Type I-E |
| Klebsiella pneumoniae | Type I-E |
| Streptococcus mutans | Type I-E |
| Pseudomonas aeruginosa | Type I-F |
| Yersinia pestis | Type I-F |
| Serratia marcescens | Type I-F |
| Geobacter sulfurreducens | Type I-U |
| Salinispora arenicola | Type I-U |
| Vibrio phage ICP1 | Type I-F |

C1 may be a Cas (eg, a Cas3 or a Cascade Cas) selected from the following types. Additionally or alternatively, C2 may be a Cas (eg, a Cas3 or a Cascade Cas) selected from the following types. Cascade Cas may be selected from the following types.

TABLE 3
Example Cas, Types and Classes

| | Type | Cas Nuclease | Target |
|---|---|---|---|
| Class 1 | I | Cas3 | DNA |
| | III | Cas10 | DNA or RNA |
| | IV | | |
| Class 2 | II | Cas9 | DNA |
| | V | Cas12 | DNA |
| | VI | Cas13 | RNA |

TABLE 4
Sequences of Dispensable Genes

| Gene | | | | | |
|---|---|---|---|---|---|
| name | | | SEQ | | |
| (T4 | | Length | ID | | |
| phage) | Protein ID | (AA) | NO | AA sequence | Function |
| 49.1 | NP_049695.1 | 51 | 1 | MDYAIKPWWAARWETVEPEPEEPVYTDEETVYNEPT | |
| | | | | INDLIDMEMGHDYSR | |
| 49.2 | NP_049696.1 | 106 | 2 | MNIENKLDVDAVLSEIIEDHDAFSENYDFDFSDYLKPIE | |
| | | | | IEDWVQDGKCQYRQCVYFSPKHNVHVAVNESRSGSY | |
| | | | | HSDWYYAVPTVELVELRERVVTQTVREWITL | |
| 49.3 | NP_049697.1 | 102 | 3 | MIELNEQIIFLGDGTEGDLEYKLYEYMIWLAKAEGIDF | |
| | | | | VVSNPYGENTVVIGGTAYEVEWRYVGLKSEEYDVTDE | |
| | | | | GKWIPIGPWFWEHGEPDFEVSSWWCEK | |
| nrdC | NP_049698.1 | 87 | 4 | MFKVYGYDSNIHKCVYCDNAKRLLTVKKQPFEFINIMP | thioredoxin |
| | | | | EKGVFDDEKIAELLTK | |
| | | | | LGRDTQIGLTMPQVFAPDGSHIGGFDQLREYFK | |
| nrdC.1 | NP_049699.1 | 80 | 5 | MTKRKEYMETAEKAVRELAIAYYNEHGKFPDRYSVLK | |
| | | | | SALTRSYKNMLSEVSD | |
| | | | | IIYKHKEQTGQSLDYDETFKQVLGIKE | |
| nrdC.2 | NP_049700.1 | 104 | 6 | MKKRLLEDIAASSNSSLIKIMAGEEDDLEMRGKIYGC | |
| | | | | DDYSPPVNWDSVMVMVERRERASKNVPNCPECGTE | |
| | | | | QVQLVHWQTNNLRYKCRHCKHRFDREENDKA | |
| nrdC.3 | NP_049701.1 | 308 | 7 | MKTRKHYIDYFDSLITKHRDYQKGHREVINNILRDFLD | |
| | | | | YIGWENHICKDTQNAYSHSLGSLLEWFKRSRLLSSVIA | |
| | | | | VNNVKKFMYPSYIETNVSNDNVVTFNIINDVKRTYLEE | |
| | | | | WFSKDSKEKFASEFSHEFNNNVNMLFKHSRRLFCHG | |
| | | | | DDRTINVNVKDWVTAKFIPSSQNGPFELLIIVCAPHEIY | |
| | | | | KNLPYMKPCEANKHNKTIRSLTYKLRTLLSKMDVVESF | |
| | | | | DDNTNYGLSLFETKVVIKLKDPNKFKPTPKPNHGNDT | |
| | | | | MKEEREYLSTRLIEVEKQIEEHTKVLKDLTAKANGLRN | |
| | | | | AIEVLK | |
| nrdC.4 | NP_049702.1 | 333 | 8 | MTRNEYIKSENSVIDDKAIPMFGQNSVLSIINQWLNSV | |
| | | | | DASIVS | |
| | | | | STKFIHEIRKISSRVDKDVIKKTFKESRLLSYLVNRDILG | |
| | | | | NFGKEIKRTKDVVGYNWF | |
| | | | | GDVNSYHLNNKEDPENIFTRRWISNFRLFKKQILKSAS | |
| | | | | KLCYGDYRQIHPLASDMIII | |
| | | | | KEYELDKNKVSIFVNYGFFTPETNQKNINKFFSIASTITR | |
| | | | | QLETALLCMETVENIHTY | |
| | | | | PFKNICGWEGYKLVISLREVKCAYSPTSKEIYQQKCDEI | |
| | | | | VNTPKEETTLEELMECLDD | |
| | | | | SPEPIEIRPEVIALEKAYKEVLEISNKAQKEYEQAKKIWE | |
| | | | | ESVNRLDRLEQALQLIK | |
| nrdC.5 | NP_049703.1 | 340 | 9 | MKTRSQIEDMVRNASYTRDVMTFLCENNLDPDKVN | |
| | | | | RVIHHFKYT | |
| | | | | NSSEWVRNFSKAGYITQMTAREQLTDFCKTIDYKNPL | |
| | | | | FVQGVGQSKVDLSSGFFNPNH | |
| | | | | YRIEWRFIALFRRQLKQILSTASRLKGSDINLKNLKFDGY | |
| | | | | TLQMEVRPLKENNRTARI | |
| | | | | SFKPNTKNSLSICECLKSQLTEAFKYMDVVAAVQSKILP | |
| | | | | RFERFKLDTTSYELDMIVS | |
| | | | | FKYEFLRKDEVTQEKKQEVQDNLNLSNYLSNDPKFW | |
| | | | | MYSSGNKDAWKFNKVNFLPVEN | |
| | | | | PSLKPVEKWHADAIEKSLKAVDAELVKATNEVLEAEKA | |
| | | | | LEQAQSRVQNLTKQRSKLNN | |
| | | | | ALNALN | |
| nrdC.6 | NP_049704.1 | 275 | 10 | MSVVINNVNAVIKSLVNKKMMNEWTVLRRGEPDKF | |
| | | | | FHRFNPTLD | |
| | | | | LNVIDRDVHAEILDKFKVDIGFGLEKHLQRTNGSGMSL | |
| | | | | SNRIMKALNKIGALSRINAS | |
| | | | | EILRNYNKGYDLYGRLMPKLSFDQMIADLWENQRRLL | |
| | | | | ALGARLAKGLDKQMIFKTNNT | |
| | | | | EDLKCFKFSTRGDDYYIRARSTDYVNMGHHLCLAFEVL | |
| | | | | KEAGTLEYSSGAKCPIGSNC | |
| | | | | ILIYRPNESSSTKLPTKPVPVRSNEKHSEQIDYFNKQIEE | |
| | | | | LIFLFNNMMMKFSDYLD | |
| nrdC.7 | NP_049705.1 | 133 | 11 | MKQLIIKRLNLLICCLCIVIAYGYYAINDYMHYKDYDVT | |
| | | | | VVNTL | |
| | | | | TGTQGKGSSLSFIAVYELKDGYRFSEYISPETYSSIEKGD | |
| | | | | NITVSLRPFDVKQTWFDN | |
| | | | | IVWFFGMALVQSICGTYIVCSILFRVIGKIE | |
| nrdC.8 | NP_049706.1 | 175 | 12 | MNAKDIFNLVNYNDGKFKSEAQSKFFNDISIGGEITVN | |
| | | | | GGQIYK | |
| | | | | SRWNWIVIIDEIGIVEIYKNTNKNRTLHWSRDTNEQYK | |
| | | | | KDKASKLSRVTQEDIEFIKK | |
| | | | | DILMYDNLIAEEQAVIDKFDEIKASREIPDFMKESVNER | |
| | | | | YTLISERIETYKKQRAERQ | |
| | | | | NTLRKFEERLNTVLA | |
| nrdC.9 | NP_049707.1 | 100 | 13 | MLYSKAREIYETKIKEAVFQFATTMRWTNDWEYSKN | |
| | | | | HKKPLVTR | |
| | | | | KAHMLVLIDREQIKAREALQNHKKAAFEWFMDNTAP | |
| | | | | ETKKAVSAWFSGKNCERSFF | |
| nrdC.10 | NP_049708.1 | 325 | 14 | MKTVTINKGMYFGKEISGTFELLGEWFPDNAPVDAQ | |
| | | | | GDGKVFVE | |
| | | | | IDGKRRGVWVYKSDISYDGVKVEEVKESYEDMKTRIN | |
| | | | | KRFNVMGMMINGIINGNIRSL | |
| | | | | IISGAAGIGKTYSLDKALNKANDIGYIEYKSINGKISGIG | |
| | | | | LYEQLWNNREENSVLLID | |
| | | | | DVDVFSDMDILNLLKAALDTGETRKVCWSTASSYLEE | |
| | | | | KGIERELEFKGTIVFITNVDI | |
| | | | | DRELDRGTKLAPHLQALVSRSVYLDLGVHTNEEIMVR | |
| | | | | VEDVILSTDMMQKRGLSDEET | |
| | | | | YKALSWMKVNVNRLRNVSLRTALYLADFIMTDKNG | |
| | | | | WEEIATGYSSEINS | |
| nrdC.11 | NP_049709.1 | 336 | 15 | MKTVVKSYFGSHLYGTSTPESDVDFKEIFVPPARDILIG | |
| | | | | NVKEH | |
| | | | | MSKNTNNTSSKNTKDNIDHELYSLKYFFKLAADGEPV | |
| | | | | ALDMLHTPPELVVKSDLPDVW | |
| | | | | KFIQDNRSRFYTTNMKSYLGYVRKQASKYGVKGSRLA | |
| | | | | ALRDVLKVVNQIPEQWVDYQE | |
| | | | | DGSIKQRRTKVEDIKHRLPENEFCEWVFHNHEKTGPQ | |
| | | | | TFYTVLGRKYQTTLSLIELKQ | |
| | | | | SLNKLDAEYGERARKAEANEGIDWKALSHACRGGLQL | |
| | | | | LEIYKTGDLVYPLQDAPFILD | |
| | | | | VKLGKHPFKTVQEFLEDVVDQVEAASTEASKNGMQQ | |
| | | | | KVDMSFWDDFLERVYLENHRSY | |
| | | | | YK | |
| mobD | NP_049710.1 | 259 | 16 | MNYTKVYNNLIKKGDNLVLLTAREHFIAHWLLAKIHY | homing |
| | | | | NSPGLIYAWWSFYNFGE | endonuclease |
| | | | | DSLGRNLKLTSRGYQLVREKFSKIHSNTMKEMWKSNE | |
| | | | | YREKRSITLSLPEIRA | |
| | | | | KISESQLEAQNKPEVKEKISKGVKAAFKRPGVKEKHSA | |
| | | | | AVKKSLNNFEAKKKQSNSS | |
| | | | | KIRQRTGKHWQDYDLLYKLWIKLNRPKRGSFGTYISKL | |
| | | | | GYPKSNYHRLIVQFNEDYER | |
| | | | | SNNENCS | |
| mobD.1 | NP_049711.1 | 181 | 17 | MMSEAKRLVLEVSPLFGELAIEKVNNMYRLTQEDDM | |
| | | | | LYFTPSEI | |
| | | | | VRLTQIEYAYTDKIVSINDEHKIHFYSSCPGFNIKSESMC | |
| | | | | LSINNWDNFITNIKYFYD | |
| | | | | STKRKHNLKWFKKCNAIITNSCNQNDETILNVSKCYEE | |
| | | | | GDVVSIRQIDDFRSHIITLK | |
| | | | | KEEAIALKTYLDSVIPTMISK | |
| mobD.2 | NP_049712.1 | 34 | 18 | MKVLFVIYVMIQYNYPMFTYNLVNNIINMIQRSM | |
| mobD.2a | NP_813809.1 | 38 | 19 | MKTEKQMFLMKLIEEYANAVSDYECSSRERGTAFPRK | |
| | | | | K | |
| mobD.3 | NP_049713.1 | 64 | 20 | MLTREQFEKIIKLAHDIEIDSYQLAVEHCEGYSYDGIEA | |
| | | | | AKRDLDKSKAKLVQYLEMIR | |
| | | | | WNNEN | |
| mobD.4 | NP_049714.1 | 60 | 21 | MISIEQADKIKELVALIRKADEERINFALSGIEEFEAKVN | |
| | | | | NAVEALDMFLDEIIDHNTRV | |
| mobD.5 | NP_049715.1 | 62 | 22 | MKIEALNQEGNIYVIINGDFFVDMDEVTSEELVELLKK | |
| | | | | RYNMCDEVATHMACAIFSLS | |
| | | | | YVVE | |
| rl.-1 | NP_049716.1 | 128 | 23 | MKFSDFSQSGKPSKADEYLGLLMAAQAYFHSAHFETK | |
| | | | | SYARHKAYDFIFSELPDLIDKF | |
| | | | | GEQYLGYSGRKYTPSIPDASKLPTDTIKMIDRILDQSNSI | |
| | | | | YKEMPPAIQSTIDDITGMFY | |
| | | | | QSKYLLSLE | |
| rl | NP_049717.1 | 97 | 24 | MALKATALFAMIGLSFVLSPSIEANVDPHFDKFMESGI | lysis |
| | | | | RHVYMLFENKSVESSEQFY | inhibition |
| | | | | SFMRTTYKNDPCSSDFECIERGAEMAQSYARIMNIKL | regulato, |
| | | | | ETE | membrane |
| | | | | | protein |
| rl.1 | NP_049718.1 | 70 | 25 | MLQLTEKQLRNLTVLqLDEIRREVGNIISALRREVSLNQ | lysis |
| | | | | SPADYTRERNFEKYLDKV | inhibition |
| | | | | KAVHRHKVNTGQK | component |
| tk | NP_049719.1 | 193 | 26 | MASLIFTYAAMNAGKSASLLIAAHNYKERGMSVLVLK | thymidine |
| | | | | PAIDTRD | kinase |
| | | | | SVCEVVSRIGIKQEANIITDDMDIFEFYKWAEAQKDIH | |
| | | | | CVFVDEAQFLKTEQVHQLSR | |
| | | | | IVDTYNVPVMAYGLRTDFAGKLFEGSKELLAIADKLIEL | |
| | | | | KAVCHCGKKAIMTARLMED | |
| | | | | GTPVKEGNQICIGDEIYVSLCRKHWNELTKKLG | |
| tk.1 | NP_049720.1 | 62 | 27 | MITREQKNEILFLVGEIISLEKDLSFEISSEYGDAETYYE | |
| | | | | LVKSIDKAENDLETYLENLTKD | |
| tk.2 | NP_049721.1 | 61 | 28 | MSLSKEQKDTLFSLIHEVMDKNSELEKVCNECGPFSA | |
| | | | | NEYEELSKEFDNKEQELIDYINSL | |
| tk.3 | NP_049722.1 | 70 | 29 | MNIHYPHPYDPKNKAVIIRQWERICRTKCPINSPHDV | |
| | | | | DKDYIGTFVEYTFIDKKGRKQHV | |
| | | | | EEYCLKVTWL | |
| tk.4 | NP_049723.1 | 155 | 30 | MIVKYIKGDIVALFAEGKNIAHGCNCFHTMGSGVAG | protein |
| | | | | QLTKAFPKILEADKLQTEWGD | contains |
| | | | | VTKLGSYSVYEKYFRTHKAYCFNLYTQFQPGPNFEYSA | A1pp |
| | | | | LMNCMLELNEFGENKLIKPT | phosphatase |
| | | | | IYMPRIGAGIGKGNWDIIEGILDTYSSKLEIVIVDWEPLL | motif |
| vs | NP_049724.1 | 115 | 31 | MTKILVLCIGLISFSASASADTSYTEIREYVNRTAADYCG | valyl-tRNA |
| | | | | KNKACQAEFAQKLIYAYKDG | synthetase |
| | | | | ERDKSSRYKNDTLLKRYAKKWNTLECSVAEEKDKAAC | modifier |
| | | | | HSMVDRLVDSYNRGLSTR | |
| vs.1 | NP_049725.1 | 181 | 32 | MRKALLAGLLAISMIMAHSSEHTFSNVQLDNMRYAY | |
| | | | | QFGEQFSKDGKYKTHKNIHKSGL | |
| | | | | GHIMAAILWQESSGGVNLKSKPKHHAYGMFQNYLPT | |
| | | | | MRARVKELGYNMTDAEIKRM | |
| | | | | LNKRSNSASWAYIELSYWLNIHKGDIRKAISSYNSGW | |
| | | | | NVKAGSKYASEVLEKANYLKNNK | |
| | | | | LLEIVND | |
| regB | NP_049726.1 | 153 | 33 | MTINTEVFIRRNKLRRHFESEFRQINNEIREASKAAGVS | RegB site- |
| | | | | SFHLKYSQHLLDRAIQ | specific RNA |
| | | | | REIDETYVFELFHKIKDHVLEVNEFLSMPPRPDIDEDFI | endonuclease, |
| | | | | DGVEYRPGRLEITDGNLWL | T4 mRNA |
| | | | | GFTVCKPNEKFKDPSLQCRMAIINSRRLPGKASKAVIK | processing |
| | | | | TQ | |
| vs.3 | NP_049727.1 | 92 | 34 | MAQLSAGFGYEYYTAPRRVSVAPKKIQSLDDFQEVVR | |
| | | | | NAFQDYARYLKEDSQDCL | |
| | | | | EEDEIAYYTQRLEQLKNLHEVRAEVSKSMNKLIRFKE | |
| vs.4 | NP_049728.1 | 88 | 35 | MIEDIKGYKPHTEEKIGKVNAIKDAEVRLGLIFDALYDE | |
| | | | | FWEALDNCEDCEFAKNYAE | |
| | | | | SLDQLTIAKTKLKEASMWACRAVFQPEEKY | |
| vs.5 | NP_049729.1 | 58 | 36 | MAKIIIEGSEDVLNAFASGLVTQANSNLMKRGIWVIL | |
| | | | | MEFILRQKFLFKAMAFMNLFV | |
| vs.6 | NP_049730.1 | 120 | 37 | MKAYQILEGTHKGTIYFEDGIQARIIVSKTFKEDSFVDP | |
| | | | | EIFYGLHAREIEIEPQPTVKIE | |
| | | | | GGQHLNVNVLRHETLEDAVKHPEKYPQLTIRVSGYAV | |
| | | | | RFNSLTPEQQRDVIARTFTESL | |
| vs.7 | NP_049731.1 | 109 | 38 | MMTDTQLFEYLYFSPKTIKNKLVNHFEILAKNNILSEFY | |
| | | | | PKQYKLQKGVEKGCRVLCT | |
| | | | | APNARLMNKIPYFTMEFIDGPFKGLITQSLMAYDSEPF | |
| | | | | LIKEQSWINLFSN | |
| vs.8 | NP_049732.1 | 219 | 39 | MRDSRQPVIRSSPSAVMIGKYRNGQFMCHGMAQTY | |
| | | | | RAYREEMRTF | |
| | | | | LTGPYLSLMNAFTHHSDARVEEICKNEYIPPFEDLLKQY | |
| | | | | CTLRLDGGRQSGKSIAVTN | |
| | | | | FAANWLYDGGTVIVLSNTSAYAKISANNIKKEFSRYSN | |
| | | | | DDIRFRLFTDSVRSFIGNKG | |
| | | | | SKFRGLKLSRILYIIDEPVKSPDMDKIYSVHIDTVHYCCN | |
| | | | | SKCCIGGITRPQFFVIGMQ | |
| denV | NP_049733.1 | 138 | 40 | MTRINLTLVSELADQHLMAEYRELPRVFGAVRKHVAN | DenV |
| | | | | GKRVRDFKISPTFILGAGHV | endonuclease |
| | | | | TFFYDKLEFLRKRQIELIAECLKRGFNIKDTTVQDISDIP | V, |
| | | | | QEFRGDYIPHEASIAISQA | N-glycosylase |
| | | | | RLDEKIAQRPTWYKYYGKAIYA | UV repair |
| | | | | | enzyme |
| lpll | NP_049735 | 193 | 41 | MKTYQEFIAEASVVKAKGINKDEWTYRSGNGFDPKTA | lpill |
| | | | | PIERYLATKASDFKAFAWEGLRWRTDLNIEVDGLKFA | internal |
| | | | | HIEDVVASNLDSEFVKADADLRRWNLKLESKQKGPKF | head protein |
| | | | | VPKAGKWVIDNKLAKAVNFAGLEFAKHKSSWKGLDA | |
| | | | | MAFRKEFADVMTKGGFKAEIDTSKGKFKDANIQYAYA | |
| | | | | VANAARGNS | |
| ipll | NP_049734 | 100 | 42 | MKTYQEFIAEARVGAGKLEAAVNKKAHSFHDLPDKD | lpll internal |
| | | | | RKKLVSLYIDRERILALPGANEGKQAKPLNAVEKKIDNF | head protein |
| | | | | ASKFGMSMDDLQQAAIEAAKAIKDK | |
| Gene | | | | | |
| name | | | | | |
| (other | | | | | |
| T-even | | Length | | | |
| phage) | Protein ID | (AA) | | AA sequence | |
| lp4 | S48009 | 157 | 43 | MKTYQEFITEAAINSQIIAESFTDLLKFKKGQKITAVLDD | lp4 protein- |
| | | | | GTEVEMDVQG | phage T2 |
| | | | | YNYAVDGKLYNKSHAKFDSFDDFVNTVEDEKTRRSIAT | |
| | | | | GDAKVLMAHGHE | |
| | | | | RIRAKQNKMGDENFALVGYQSGKQTYGYQRTATMY | |
| | | | | NKNGKIAFVNSKGSIQYVKSFK | |
| ACG- | YP_ | 71 | 44 | MKTRSQIEDMVRNASYTRDAMTFLCENNLDPDRVNS | protein |
| C40_ | 006986639 | | | SIHFKYMNSSEWLR HFDKAGYITQMTAREQLTHSN | similar |
| 0086 | | | | | to NrdC.5 |
| Vs.7 | NP_861811 | 102 | 45 | MMTTIEVFEYCYNSPVCNKRALVENYEIYHFKPKRYRL | Vs.7 protein |
| | | | | TKGPFAGQQVLCTAPNARLMTSIPHFKMEFIDGPF | RB69 phage |
| | | | | KGLITQSLMAFNSDPFLIKEKTWINLFSN | |
| PI26_ | YP_ | 99 | 46 | MAQILSGFGTHYEASRRITESNPFGLVPKHKKIQSLDD | protein- |
| gp112 | 009100654 | | | FENRLWALFNEYKAYLKEDADDCLEEDEIAYYEQRLEQ | Shigella |
| | | | | LKNFHQVRDEVSKAVKKLIPFKE | phage |
| | | | | | Shf125875 |
| APCEc01_ | YP_ | 115 | 47 | MTKMLALIVGLVSFNALANTTYTDVTEYTNRTASDYC | valyl-tRNA |
| 190 | 009225150 | | | GKSQECKVDFSQKLLYAYKDGEKDGASSRFKASTLIKR | synthetase |
| | | | | YYKKWQILECSVAEPKDKAACNSMVDRLVDSYNRGL | modifier |
| | | | | AASD | Escherichia |
| | | | | | phage |
| | | | | | APCEc01 |
| rl.1 | NP_861800 | 70 | 48 | MQQLNERQLRNLTVTQLDEIRRELGHSISHLNEDIRQT | rl.1 protein- |
| | | | | GSKADYTRKRKLEKYLADVKAV | Escherichia |
| | | | | QRRKINTGQN | phage RB69 |
| rl | QHR76563 | 100 | 49 | MALRAIAVMAMLGFFAATTPIVGTAYVDPYFDNFME | lysis |
| | | | | SGIKNVYTLFEIQNVENSEKFYKY | inhibition |
| | | | | MAKHYKNSPCDDAFECHEQGIKTARQFAEFMKIKLEP | regulator- |
| | | | | TSI | Escherichia |
| | | | | | phage moskry |
| MobD.2a | NP_861798 | 59 | 50 | MTRKEKISKLMFLIEEYANSVSDWENAHGCEDGDIDI | MobD.2a |
| | | | | NRAMIKKMADAHTELQMYVNEIM | protein- |
| | | | | | Escherichia |
| | | | | | phage RB69 |
| FDI17_ | YP_ | 55 | 51 | MLYDYTGKSEDGVLELLPESAEDDDMVVIYCVGCQS | protein |
| gp010 | 009608299 | | | MHDEIFKREPRNCWHRSMR | [Escherichia |
| | | | | | phage ST0] |
| JS09_ | YP_ | 37 | 52 | MAQEHEMIYYLEPWAWLTMAVGPVLIGLFFSWLAR | protein |
| 078 | 009037401 | | | KL | JS09_078- |
| | | | | | Escherichia |
| | | | | | phage |
| | | | | | vB_EcoM_JS09 |
| SP18_ | YP_ | 71 | 53 | MLYDPSGNSENGVIVLEPEHPVGDVYRPVEVCECNFV | protein |
| gp102 | 003934727 | | | KGNTGGISIEQDDDVIYLDASQV | SP18_gp102- |
| | | | | EALYSILKHNR | Shigella phage |
| | | | | | SP18 |
| PhAPEC2_ | YP_ | 36 | 54 | MKLLLIAYVVVQYNYPMFTYNMVNGIVNLIETSMVK | protein |
| 92 | 009056684 | | | | PhÅPEC2_92- |
| | | | | | Escherichia |
| | | | | | phage |
| | | | | | VB_EcoM_ |
| | | | | | PhAPEC2 |
| FDI17- | YP_ | 59 | 55 | MTREQANKLMDLIHDLREADSDLNDVAYHAVNDNG | protein- |
| gp014 | 009608303 | | | DFYENQVDACQNALVNFVETLIGE | Escherichia |
| | | | | | phage ST0 |
| MobD.1 | NP_861795 | 176 | 56 | MKTVIETTELFGDLCIEKRGYAYVLTQEDDAVTILPMEL | MobD.1 |
| | | | | DKILKLNPPGHASVINIDEDL | protein- |
| | | | | QVRFYHGLYSGVNIETEDECFSINNWKTFVTKVKEFM | Escherichia |
| | | | | ESETVKKAKLQWAKCRNAFITNQ | phage RB69 |
| | | | | DRPDYTTVLGVNPSYEDGDVVVIRQIDDLRQHIITLDK | |
| | | | | DEAVALKAYLDSIIPTLK | |
| PhAPEC2_ | YP_ | 157 | 57 | MIINENSWHFKIYAAFNSTWNRPKTLCAYFWKTFLPV | protein |
| 90 | 009056682 | | | LFVSIIGFAILAAATIVGQEILTKFFVFSSLWTLVPASIG | PhAPEC2_90- |
| | | | | VGVLFIALCVGISIVLVLGIPWLIDLYRERKYYKEVELIA | Escherichia |
| | | | | QNQERIKNGLEPIERKKSLIGEYLKARKEKVCPTLEYKAK | phage |
| | | | | | vB_EcoM_ |
| | | | | | PhAPEC2 |
| HX01_ | YP_ | 95 | 58 | MDFMQKIKAAVELYAHRMVSSTMQYSEEVRQDSSLK | protein |
| 083 | 006907236 | | | GKTKALITLDRLQKNALELFQYDKDAYKKEQEEKLKSEK | HX01_083- |
| | | | | LQKVPKSVWFSGKNTEKSFF | Escherichia |
| | | | | | phage HX01 |
| FDI17_ | YP_ | 331 | 59 | MSITSDAVKHEINGLCASQIKVWFKDHNDRRALNYEQ | protein- |
| gp022 | 009608311 | | | IAIQVHQLLTSKFDFHMPSEIYT | Escherichia |
| | | | | NLPRALIKACKGYGKSMADGIFSALNKLNVLSMINSN | phage ST0 |
| | | | | NILRNLGRTDLFGREQCKMPLKW | |
| | | | | IVAHLNENQTRFMAVSARLQMGLDRQMNFKTHPRY | |
| | | | | SGSTDFYKCELQKVAGKTVLVVRVT | |
| | | | | FEGRRQGYFDLIGSGLKHCGFIDEVDIKRDSIGYRIGYV | |
| | | | | CTLKEEISGPAPGLKDVRYKD | |
| | | | | ELVEEIKEVEYDINQEDALKDLIDELNQDVIPVQKFESK | |
| | | | | HAEQIKYFEALINELNVTIQQ | |
| | | | | TDDEISRLAGINGKNKTERANLIQVVKLLKE | |
| JS09_090 | YP_ | 311 | 60 | MASITRRELIKEFTSNAYTPGPNPIRMPVKTPEEIEEICS | protein |
| | 009037413 | | | YYGITSRKFTQFECVINTPV | JS09_090- |
| | | | | KEFIKNGEFSRLLAKQKLRNFCIGGEGQYFAKQFRNNL | Escherichia |
| | | | | NNLMAIASRIRPQAINMKHLKY | phage |
| | | | | TYDKLEIMVISENEFTLTYKPKNDNVARVFHQALEVLG | vB_EcoM_JS09 |
| | | | | DNIHQIKAIYSRELNIINPIKN | |
| | | | | KGHIAIQARVSIGKKVPAPWWHKDSEYYQNLKKENA | |
| | | | | TFEASITTTPIGNAEVQGAYFTAP | |
| | | | | SIYTSPSTKLEISNGLLPAEDYHYNQIKATMDKLSVELD | |
| | | | | KAQAKLRDAQSVVINLQLQYS | |
| | | | | KLFNAMNALKS | |
| thior- | ASZ76276 | 322 | 61 | MKTVTINKGIYFGKEISGTFELLGEWFPDNAPVDAQG | thioredoxin- |
| edoxin | | | | DGKVFVEIDGKRRGVWVYKSDIS | Salmonella |
| | | | | YDGVKVEEVKESYEDMKTRINKRFNVMGMMTNGIIN | phage SG1 |
| | | | | GNIRSLIISGAAGIGKTYSLDKAL | |
| | | | | NKANDIGYIEYKSINGKISGIGLYEQLWNNREENSVLLI | |
| | | | | DDVDVFSDMDILNLLKAALDT | |
| | | | | GETRKVCWSTASSYLEEKGIEREFEFKGTIVFITNVDID | |
| | | | | RELDRGTKLAPHLQALVSRSV | |
| | | | | YLDLGVHTNEEIMVRVEDVILSTDMIMQKRGLSDEETY | |
| | | | | KALSWMKVNVNRLRNVSLRTALY | |
| | | | | LADFIMTDKNGWQEIAEVTLLK | |
| pSs1_ | YP_ | 244 | 62 | MKTVMKSYFGSHLYGTSTPESDVDFKEIFVPPARDILI | protein |
| 00108 | 009110916 | | | GNVKEHMSKNTNNTSSKNTKDD | pSs1_00108- |
| | | | | IDHELYSLKYFFKLAADGETVALDMLHTPPELVVKSDLP | Shigella |
| | | | | DVWKFIQDNRSRFYTTNMKSY | phage pSs-1 |
| | | | | LGYVRKQASKYGVKGSRLAALRDILKVVNQIPEQWVD | |
| | | | | YQEDGSIKQRRTKVEDIKHRLPE | |
| | | | | NEFCEWVFHNHEKTGPQTFYTVLGRKYQTTLSLIELKQ | |
| | | | | SLNKLDAEYGDVLVRLKRMRAL | |
| | | | | TGKL | |
| HY01_ | YP_ | 161 | 63 | MHILMQCLGNKISDFNHKRVNDYIKDKLEIKDILFRGL | protein |
| 0089 | 009148540 | | | TIEEVLSYKFEVNRRIKFKRITSFSSESHIAETFAAERYM | HY01_0089- |
| | | | | TNVLVVLKNANVEDYSTAMIDILENLIAIEESGQTDDDK | Escherichia |
| | | | | LNKLYDNLCMVDYEREFLLPINSELIVKNIYFDSKKNM | phage HY01 |
| | | | | HIVEME | |
| ACQ28_ | YP_ | 180 | 64 | MKRLVLEVSSLFGELAIEKVNNMYRLTQEDDMLYFTP | protein- |
| gp097 | 009153700 | | | SEIIHLTQIEKPYTDKIVSINDEHKIHFYSLCPGFNIESE | Yersinia |
| | | | | SICLSINNWDNFITYIKYFYYSNERKHSLKWLKNCNAIIT | phage PST |
| | | | | NACDQCDQNDETVLNVSKCYEEGDVLTIRQIDDSRAHI | |
| | | | | VTFTKDEAIALKTYLDSVIPTMISK | |
| AR1_ | YP_ | 165 | 65 | MKKYFTIYKTEILNLKTGIKKYYYGRHITNNINDSYVGS | site-specific |
| 097 | 009167908 | | | GNYIERVKVSNDHILSKTVLEVFDNYDKMIVQAEILYIM | intron-like |
| | | | | AGKLKYGKNCVNLSYGGEGGKVADETRIKCKKHSINL | DNA |
| | | | | WKNPNHIQHMKKKMKEVTSTPEAKLNTSIKTSQRYK | endonuclease- |
| | | | | DPVLLEKLVILLKRN | Escherichia |
| | | | | | phage AR1 |
| phiE142_ | ALY07947 | 93 | 66 | MKTYNEFLSESILNEATDTFATKLGAALVEAESLLARISE | protein |
| 142 | | | | LASKIDTRKFERTSDIVKLE | phiE142_142- |
| | | | | ALLRMCDKPEEIAKNGSLMKQRLEKYISGASEK | Escherichia |
| | | | | | phage phiE142 |
| homing | YP_ | 239 | 67 | MKKYFTIYKTEILNLKTGVKKYYYGRHITNNINDSYVGS | homing |
| endo-- | 009277738 | | | GNYIERVKVSNDHILSKTVLEVFDNYDKMVQAEILYIM | endonuclease- |
| nuclease | | | | AGKLKYGKNCVNLSYGGEGGKVADETRIKCKKHSINL | Shigella phage |
| | | | | WKNPNHIQHMIKKKMKEVTSTPEAKLNTSIKTSQRYK | SHFML-11 |
| | | | | DPVFVRKVSNIIKKKLSNPAVKEKHINGIKAAKRSHLTI | |
| | | | | WQYEDELFELWKETKCGWRKFTKEAIKHGYPNEPYRS | |
| | | | | MVSTFQKRHLEES | |
| AVU02_ | YP_ | 73 | 68 | MKTVMKSYFGSHLYGTSTPESDVDFKEIFVPPARDILI | protein- |
| gp226 | 009197358 | | | GNVKEHMSKNTNNTSSKTLKMILTMNYTVLNISLN | Escherichia |
| | | | | | phage slur07 |
| thiored- | YP_ | 152 | 69 | MKTVTINKGIYFGKEISGTFELLGEWFPDNAPVDAQG | thioredoxin- |
| oxin | 009277471 | | | DGKVFVEIDGKRRGVWVYKSDISYDGVKVEEVKESYE | Shigella phage |
| | | | | DMKTRINKRENVMGMMINGIINGNIRSLIISGAAGV | SHFML-11 |
| | | | | GKKDSFDKTENKTKDIWKIEYKNINGKISGIGLFEQLW | |
| | | | | NCLF | |
| thiored- | YP_ | 155 | 70 | MDILNLLKAALDTGETRKVCWSTASSYLEEKGIDREFE | thioredoxin- |
| oxin | 009277739 | | | FKGTIVFITNVDIDRELDRGTKLAPHLQALVSRSVYLDL | Shigella phage |
| | | | | GVHTNEEIMVRVEDVILSTDMMQKRGLSDEETYKALS | SHFML-11 |
| | | | | WMKVNVNRLRNVSLRTALYLADFIMTDKNGWEEIAE | |
| | | | | TVLLK | |
| AREG1_ | YP_ | 64 | 71 | MSLSKEQKDRLFSLIHEVMDKNNELEKVCDESDYGDA | protein |
| g106 | 009281446 | | | ETYYELVKSIDKAENDLETYLENLTKD | AREG1_g106- |
| | | | | | Escherichia |
| | | | | | phage UFV- |
| | | | | | AREG1 |
| AREG1_ | YP_ | 57 | 72 | MNNFELRYEVLRNIDNLIELAVNKGFAIGIGQKDTTFID | protein |
| g107 | 009281447 | | | KNKRKQHVEEYCLKVTWL | AREG1_g107- |
| | | | | | Escherichia |
| | | | | | phage UFV- |
| | | | | | AREG1 |
| AREG1_ | YP_ | 70 | 73 | MKVHYPHPFDPKNKAEHIRHWTETRVTKCPIKSQHNT | protein |
| g109 | 009281449 | | | DKWYIGEYVEYTFIDKNKRKQHVEEYCLKVTWL | AREG1_g109- |
| | | | | | Escherichia |
| | | | | | phage UFV- |
| | | | | | AREG1 |
| internal | YP_ | 101 | 73 | MKTYQEFIVEAKRREDELPFVSKTFDGTLADFKKIPND | internal head |
| head | 009281461 | | | KLAKLVGSISETPMNYDRFYPCDFVNDKLALIFEENND | protein- |
| protein | | | | LLLSYVDLITKNYNNPKIKGKQITN | Escherichia |
| | | | | | phage UFV- |
| | | | | | AREG1 |
| MUFV13_ | YP_ | 50 | 74 | MGTFLGGDGKEINTYAKRSEIAYVVASTKVDVDDIGY | protein |
| g126 | 009290391 | | | DFNNFGIISGGKA | vBEcoMUFV13_ |
| | | | | | g126- |
| | | | | | Escherichia |
| | | | | | phage |
| | | | | | vB_EcoM- |
| | | | | | UFV13 |
| endo- | QEG05969 | 179 | 75 | MRTFLTGPYLSLMNAFTHHSDARVEEICKNEYIPPFED | endo |
| ribo- | | | | LLKQYCTLRLDGGRQSGKSTAV | ribonuclease- |
| nuclease | | | | TNFAANWLYDGGTVIVLSNTSAYAKISANNIKKEFSRY | Shigella |
| | | | | SNDDIRFRLFTDSVRSFIGNKG | phage JK38 |
| | | | | SKFRGLSLSRILYHIDEPVKSPDMDKIYSVHIDTVHYCQN | |
| | | | | SKCCIGGITRPQFFVIGMQ | |
| MUFV13_ | YP_ | 42 | 76 | MYQEFIVEADAKGPKFKVKAFIGDAEDFGSLKRNGYFL | protein |
| g127 | 009290392 | | | RWRW | vBEcoMUFV13_ |
| | | | | | g127- |
| | | | | | Escherichia |
| | | | | | phage |
| | | | | | vB_EcoM- |
| | | | | | UFV13 |
| HY03_ | YP_ | 98 | 77 | MKTRSQIEDMVRNASYTRDVMTFLCENNLDPDKVN | protein |
| 0110 | 009284100 | | | RVIYHFKYTNSSEWLRHFSKAGYITQMTAREQLTDFCK | HY03_0110- |
| | | | | TIDYKNPLFVRGVGQSKTDLSTGFF | Escherichia |
| | | | | | phage HY03 |
| thiored- | ASZ76271 | 340 | 78 | MKTRSQIEDMVRNASYTRDVMTFLCQNNLDPDKVN | thioredoxin- |
| oxin | | | | RVIHRFKYTNSSEWLRHFSKAGYIT | Salmonella |
| | | | | QMTAREQLTDFCKTIDYKNPLFVRGVGQSKVDLSSGF | phage SG1 |
| | | | | FNPNHYRIEWRFIALFRKQLKQI | |
| | | | | LSIASRLKGSDINLKNLKFDGYTLQMEVRPLKENNRTA | |
| | | | | RISFKPNTKNSLSICECLKSQL | |
| | | | | TEAFKYMDVVAAVQSKILPHFERNWEHTTTYELDMIV | |
| | | | | SFKYEFLRKDEIVQEKKQEVQNT | |
| | | | | LNSSNYSSNDPKFWMYSSSNIDACKLNKVSFLPTENS | |
| | | | | NFKPVEKWHADAIEKSLKAVDDE | |
| | | | | LVKATNEVLEAEKVLKQAQSRVQNLTKQRSKLNNALN | |
| | | | | ALN | |
| HY03_ | YP_ | 308 | 79 | MTRKHYIDYFDSLITKHRNYQIGRRAVINNILRDFLQYV | protein |
| 0112 | 009284102 | | | GQENHICKDTQNAYSHSLGNLLQWFKRSRLLSSTVAS | HY03_0112- |
| | | | | DNIKNFMKPSFIKSETSITDLVEFTIVNDVKKTHLADWL | Escherichia |
| | | | | STIPETKFADKFACQFNDQVNMLFKHARKLFTAGDDR | phage HY03 |
| | | | | TNTVHVKDWVIADEVTRKPGGSSVLINIQVPYYYSHN | |
| | | | | LGTMTAREINKHNKIIRSLSYKLCTMVEMMDVVEMY | |
| | | | | DETEDNGSMLYSSRILIKLKNPNTYKQVVKEPKAEKAD | |
| | | | | NLSEEREYLNARLIEVEAQIAEHTKLLKALNAKANGLRN | |
| | | | | AIEVLK | |
| thiored- | YP_ | 296 | 80 | MSVVINNVNAVIKSLVNKKMMNEWTVLRRGEPDKF | thioredoxin- |
| oxin | 009279086 | | | FHRFNPTLDLNVIDRDVHAKILDKFKVDIGFGLEKHLQ | Shigella |
| | | | | RTNGSGMSLSNRIMKALNKIGALSRINASEILRNYNKG | phage |
| | | | | YDLYGRLMPKLSFDQMIADLWENQRRLLALGARLAK | SHFML-26 |
| | | | | GLDKQMIFKTNNTEDLKCFKESIRGDDYYIRARSTDYV | |
| | | | | NMGHHLCLAFEVLKEAGTLEYVSGAKCPIGSSCILIYRP | |
| | | | | DESSSTKLPTKPVPVRSNEKHSEQIAYENKQIEELNISIQ | |
| | | | | QYDDEIFRLSGLSSKAKSEREKLIKIVDLLKS | |
| Bl057_ | YP_ | 120 | 81 | MAAILWQESSAGVNLKSKPKHHAYGMFQNYLPTMR | protein- |
| gp211 | 009279109 | | | ARVKELGYNMITDAEIKRMLNKRSNSASWAYIELSYWL | Shigella |
| | | | | NIHKGDIRKAISSYNSGWNVKAGSKYASEVLEKANYLK | phage |
| | | | | NNKLLEIVND | SHFML-26 |
| Sf22_ | YP_ | 409 | 82 | MSVVINNVNAVIKSLVNKKLNEWTVLRRGEPDKFFHR | protein |
| gp260 | 009615012 | | | FNPTLDLNVIDRDVHAEILDKFKVDIGFGLDKHLQRTN | Sf22_gp260- |
| | | | | GSGMGLSNRIMKALNKIGALSRINASEILRNYNKGYDL | Shigella |
| | | | | YGRLMPKLSFDQMIADLWENQRRLLALGARLAKGLD | phage Sf22 |
| | | | | KQMIFKTNNTEDLKCFKFSIRGDDYYIRARSTDYVNM | |
| | | | | GHHLCLAFEVLKEAGTLEYSSGAKCPIGSNCILIYRPDES | |
| | | | | SLTKFPTKPVPVRSNEKHSEQIDYFNKQIEELNISIDKQ | |
| | | | | MIFKTNNTEDLKCFKFSIRGDDYYIRARSTDYVNMGH | |
| | | | | HLCLAFEVLKEAGTLEYSSGAKCPIGSNCILIYRPDESSL | |
| | | | | TKFPTKPVPVRSNEKHSEQIDYFNKQIEELNISIQQYDD | |
| | | | | EIFRLSGLSSKAKSEREKLIKIVDLLKS | |
| Vs.5 | YP_ | 73 | 83 | MAKIIIEGSKDVLNAFAEWFSNSGEQQFNEAWTMGD | protein- |
| | 007004498 | | | IDGIYPTTEISVQGYGIHEPIRLVEYDLCTGEEVKYD | Escherichia |
| | | | | | phage ime09 |
| SH7_ | APC45000 | 129 | 84 | MMLEGTDYIHDYRGSAVYVGDEVAVYYGYGTLMTAK | protein |
| 0078 | | | | VIQIKNNRAKLEVYYSNGEKSISKWKYGDCMVKLGVN | SH7_0078- |
| | | | | MIYDISVSRTPSMVTIPAEELDRLHKIEELLWEIESDLPS | Shigella |
| | | | | GLESWIDYEELNKLRN | phage SH7 |
| BN81_ | YP_ | 66 | 85 | MTTLNKRFGKAKSGCAAERRYVFAQLEDVEYQINQV | protein of |
| 099 | 009149338 | | | HITGIEPMCGLEALREVRDGYRRDIEEMSK | unknown |
| | | | | | function- |
| | | | | | Yersinia |
| | | | | | phage phiD1 |
| RB3_114 | YP_ | 70 | 86 | MVMSQTSILKNAHCEKCKWPVVFALCNDEMACDFD | protein |
| | 009098500 | | | YWCYCSNKGCINHKGEGFYSGFYPYPDFVKEGKPK | RB3_114- |
| | | | | | Escherichia |
| | | | | | phage RB3 |
| pSs1_ | YP_ | 58 | 87 | MNDDLKYQLLRELDVLIELSAQKGFIIGSGQKDPNGHS | protein |
| 00124 | 009110932 | | | IVAVMNQKRVILKLLGIDIL | pSs1_00124- |
| | | | | | Shigella |
| | | | | | phage pSs-1 |
| DNA | YP_ | 159 | 88 | MEKTVLTCHTGGGGEGGKVADETRIKCKKHSINLWK | site-specific |
| endo- | 009098481 | | | NPNHIQHMKKKMKEVTSTPEAKLNTSIKTSQRYKDPV | intron-like |
| nuclease | | | | FVRKVSNIIKKKLSNPVVKEKHINGIKAAKRSHLTIWQY | DNA |
| | | | | EDELFELWKKTKYGWRKFTKEAIKHGYPNEPYRSMVS | endonuclease- |
| | | | | TFQKRHLEES | Escherichia |
| | | | | | phage RB3 |
| RB3_096 | YP_ | 96 | 89 | MKKYFTIYKTEILNLKTGIKKYYYGRHITNNINDSYVGS | protein |
| | 009098482 | | | GNYIERVKVSNDHILSKTVLEVFDNYDKMVQAEILYIM | RB3_096- |
| | | | | AGKLKYGKNCVNLSYGGGG | Escherichia |
| | | | | | phage RB3 |
| RB3_102 | YP_ | 61 | 90 | MTSEQAFKLRELIETYSKAVHTATVIDESAFSGHANKIK | protein |
| | 009098488 | | | YKTLMEEAKVNLDSYIETLIGE | RB3_102- |
| | | | | | Escherichia |
| | | | | | phage RB3 |
| ECML134_ | YP_ | 87 | 91 | MTVYVDVLMINHGWKLRGHPTKNCHMFTDGDIEELH | protein |
| 093 | 009102568 | | | EMAEAIGMKRSWFQDKRIKHYDLHARRRQKAVELGA | ECML134_093- |
| | | | | VEVSRREAVKIWRTLK | Escherichia |
| | | | | | phage ECML- |
| | | | | | 134 |
| ACG- | YP_ | 49 | 92 | MKITPIEVKKLIDTEEISECFESFLEDATEDNAVYLAQKI! | protein ACG- |
| C40_0093 | 006986646 | | | ETYLEKNQ | C40_0093- |
| | | | | | Escherichia |
| | | | | | phage |
| | | | | | vB_EcoM_ACG- |
| | | | | | C40 |
| FE6_099 | AUV60962 | 173 | 93 | MFISSGSGLIRVEFKNDIFLIQGDDIIKMSYDEIKKICHA | protein |
| | | | | LESHGKVNAVIDIGDLWVTLYEVSEGFNIEDENNILAID | FE6_099- |
| | | | | KRSDLFDVLKVYEQSNGGRKAVLIYQKPHSCGTASIISD | Escherichia |
| | | | | IEDETDTYMCVLKAGGDRHPDFISIRQNNEEISLSKSEA | phage |
| | | | | EAMIKYLTTVTPSMKG | vB_EcoM- |
| | | | | | fFiEco06 |
| NrdC.2 | BBC14426.1 | 98 | 94 | MKKRLLEDIAEASDFPEWTPCAGFDKGLLVTDDLDFK | NrdC.2 |
| | | | | PPAWDAIMAMVERRERASKNVPNCPECGTEQVQLV | protein- |
| | | | | HWQTSNLRYKCRHCKHRFNREENDKA | Escherichia |
| | | | | | phage PP01 |
| gp49.2 | BBC14696 | 67 | 95 | MTIENKLDVDAVLSENIEDHDAFSENYDFDFSDYLKPIEI | gp49.2 |
| | | | | EDWVQDGKCQYRQCVYFSPKHNVHGCK | protein- |
| | | | | | Escherichia |
| | | | | | phage T2 |
| NrdC.4 | BBC14704 | 155 | 96 | MFGQNSVLSIINQWLNSVDAGIVSSAKFIHEIRKISSRV | NrdC.2 |
| | | | | DKDVIKKTFKESRLLSYLVNRDILGNFGKEIKRTKDVVG | protein- |
| | | | | YNWFGDVNSYHLNVKEDPENIFTRRWISNFRLFKKQI | Escherichia |
| | | | | LKSASKLCYGDYRQIHPLASDMINIKEYELDKNKAAFCEL | phage T2 |
| NrdC.6 | BBC14707 | 182 | 97 | MMNEWTVLRRGEPDKFFHRFNPTLDLNVIDRDVHA | NrdC.6 |
| | | | | EILDKFKVDIGFGLEKHLQRTNGSGMSLSNRIMKALNK | protein- |
| | | | | IGALSRINASEILRNYNKGYDLYGRLMPKLSFDQMIAD | Escherichia |
| | | | | LWENQRRLLALGARLAKGLDKQMIFKTNNTEDLKCFK | phage T2 |
| | | | | FSTRGDDYYVRARSTDYVNMGHHLCLAFEVLKEA | |
| NrdC.7 | AYD82688 | 99 | 98 | MKQLIIKRLNLLICCLCIVIAYGYYAINDYMHYKDYDVT | NrdC.7 |
| | | | | VVNTLTGTQGKGSSLSFIAVYELKDGYRFSEYISPEMYS | protein- |
| | | | | SIEKGDNYCKFTSFRRKTDIV | Escherichia |
| | | | | | phage T2 |
| Shfl2p0 | YP_ | 55 | 99 | MKNLISFGVKPWWAARWETVEPEPEEPVYTDEETVY | protein |
| 83 | 004414980 | | | NEPTINDLIDMEMGHDYSR | Shfl2p083- |
| | | | | | Shigella |
| | | | | | phage Shfl2 |
| N/A | ASZ77225 | 119 | 100 | MFISSGSGLIRVEFKNDIFLIQGDDIIKMSYDEIKKICHA | protein- |
| | | | | LESHGKVNAVIDIGDLWVTLYEVSEGFNIEDENNILAID | Escherichia |
| | | | | KRTDLFDVLKAYEQSKWRKKSCIDLSKTAFMWNCFN | phage ECO4 |
| | | | | HFKY | |
| N/A | ASZ77226 | 105 | 101 | MKRLVLEVSPLFGELAIEKVNNMYRLTQEDDMLYFTP | protein- |
| | | | | SEIVRLTQIEYAYTDKIVSINDEHKIHFYSSCPGFNIKSE | Escherichia |
| | | | | SMCLSVIHWDSFIAKIKYFILMKENIV | phage ECO4 |
| mobD.2a | YP_ | 57 | 102 | MKTEKQMFLMKLIEEYANAVSDYECSSRERGTAFAKE | mobD.2a |
| | 002854439 | | | EMKIMVDAHTKLQNFIENVI | protein- |
| | | | | | Escherichia |
| | | | | | virus RB14 |
| JB75_ | AXC34095 | 153 | 103 | MIINESSWHYKLFKMFNDEWKRPKTLCAYFWSIVIPT | membrane |
| 0170 | | | | FFVSFFGCTILAGLTIICAEIMQKWLIFGSLWTLIPSAFI | protein- |
| | | | | LAILLVLLIIGSFVIPAQLHEKYKDYKWKKDYALHVENID | Escherichia |
| | | | | RAYKGLPPIQPKKSIIVEFLKARKAKVCPVIEYKAE | phage |
| | | | | | vB_EcoM_JB75 |
| Sf22_ | YP_ | 180 | 104 | MKAVIETTDLFGDLVIEKRGNVYVLTQEDDSITILPMEL | protein |
| gp1 | 009614753 | | | AAILSYAPSGVTGVTNITGDL | Sf22_gp1- |
| | | | | QVRFYRGLYGGNIETEFMCLSINNWATFVAKVKEFLES | Shigella |
| | | | | ETAETPEKAKLQWAKDRGANII | phage Sf22 |
| | | | | NPVAANYTTTLDVKTSYEDGDVVLVRQNDDSRAHIVT | |
| | | | | FTKDEAIALKTYLDSVIPTMISK | |
| N/A | AYD85352 | 57 | 105 | MTKRKEYMETAEKAVRELAIAYYNEHGKFPDRYSVLK | protein- |
| | | | | SALTRSYKNMLSEVSDIIQT | Escherichia |
| | | | | | phage |
| | | | | | vB_vPM_PD112 |
| NrdC.2 | AYD82682 | 68 | 106 | MVAMISPPVNWDSVMVMVERRERASKNVPNCPEC | NrdC.2 |
| | | | | GTEQVQLVHWQTSNLRYKCRHCKHRFDREENDKA | protein - |
| | | | | | Escherichia |
| | | | | | phage T2 |
| N/A | AYD82696 | 57 | 107 | MFISSGSGLIRVEFKNDIFLSQGDDIIKMSYDEIKKICHT | protein- |
| | | | | LESRGKVNAVLTLVIYG | Escherichia |
| | | | | | phage T2 |
| N/A | AYD82697 | 97 | 108 | MICYITPSEIVRLTQIEYAYTDKIVSINDEHKIHFYSSCP | protein- |
| | | | | GFNIKSESMCLSINNWDNFITNIKYFYDSTKRKHNLKWF | Escherichia |
| | | | | KNVMLLLLTPVIRMMKLF | phage T2 |
| Sf23_ | ATE86631 | 88 | 109 | MGFPKLEVGDLVLTKLWNDAQSVEICQYRGATGNLM | protein |
| gp221 | | | | YTIYNPEILLECHLERFIKDTDSMPYSVSIVRKSDTKEYS | Sf23_gp221- |
| | | | | KILEQIRSNKKD | Shigella |
| | | | | | phage Sf23 |
| N/A | AYD82698 | 44 | 110 | MSESKRINMIKRLVLEDSVLFGELAIEKVNNMYRLTQE | protein- |
| | | | | DDMLYYA | Escherichia |
| | | | | | phage T2 |
| DNA | QBO63362 | 195 | 111 | MTRINLTLVSELTDQHLMAEYRELPRVFGIVRKHVAN | pyrimidine |
| glyco- | | | | GKRVKDFKISSEFILGSGHVTFFYDKLEFLRKRQSDIITE | dimer DNA |
| sylase | | | | CLKRGFSIKDTEVPDISDIPTRGKILMAQLSAGFGYEYY | glycosylase- |
| | | | | TAPRRVSAAPKKIQSLDDFQEVVRKAFQDYARYLKEDS | Escherichia |
| | | | | QDCLEEDEIAYYEQRLEQLKNLHEVRAEVSKSMNKLIR | phage |
| | | | | FKE | vB_EcoM_G2540 |
| G2133_ | QBO60866 | 58 | 112 | MYQEFIVEADAKGSKFKVKAFIGDVEDFGSLKRNGYSF | protein |
| 00127 | | | | LGGDGKEINTYANVVKLLML | G2133_00127- |
| | | | | | Escherichia |
| | | | | | phage |
| | | | | | vB_EcoM_G2133 |
| G4498_ | QBO64167 | 84 | 113 | MYQEFIVEADAKGSKFKVKAFIGDAEDFGSLKRNGYSF | protein |
| 00127 | | | | LGGDGKEINTCAKRSEIAYVVASPKVDVDDIGYDFNNF | G4498_00127- |
| | | | | GIISGGKA | Escherichia |
| | | | | | phage |
| | | | | | vB_EcoM_G4498 |
| RB32O | YP_803024 | 71 | 114 | MMLEGTDYIHDYRGSAVYVGDEVAVYYGYGTLMTAK | protein |
| RF082c | | | | VIQIKNNRAKLEVYYSNGEKSISKWKYGDCMVKLG | RB32ORF082c- |
| | | | | | Escherichia |
| | | | | | virus RB32 |
| ACG- | YP_ | 157 | 115 | MKTYQEFITEAAINSQIIAESFTDLLKFKKGQKITAVLDD | protein ACG- |
| C40_0123 | 006986677 | | | GTEVEMDVQGYNYAVDGKLY | C40_0123- |
| | | | | NKSHAKFDSFDDFVNTVEDEKTRRSIVTGDAKVLMAH | Escherichia |
| | | | | GHERIRAKQNKMGEDNFALVGYQ | phage |
| | | | | SGKQTYGYQRTATMYNKNGKIAFVNSKGSIQYVKSFK | vB_EcoM_ACG- |
| | | | | | C40 |
| G4507_ | QBO66094 | 39 | 116 | MKTYQEFITEAAINSQIIAESFTDLLKFKKRTENYCCIG | protein |
| 00127 | | | | | G4507_00127- |
| | | | | | Escherichia |
| | | | | | phage |
| | | | | | vB_EcoM_G4507 |
| G8_00091 | QBQ79975 | 158 | 117 | MDFFTPEANQKNINKFFSIASTITRQLETALLCMETVE | protein |
| | | | | NIHTYPFKNICGWEGYKIVISLREVKCAYSPTDKEIYQQ | G8_00091- |
| | | | | KCDEIVNTPKEETTLEELMECLDDSPEPVEIRPEVIALEK | Escherichia |
| | | | | AYKEVLEISNKAQKEYEQAKKIWEESVNRLDRLEQALQ | phage |
| | | | | LIK | vB_EcoM_G8 |
| tk.4 | ASZ77242 | 159 | 118 | MIVKYIKGDIVALFLQGNIIAHGCNCFHTMGSGVAGQ | protein- |
| | | | | LARAYPKILEIDKTTTEYGSRDKLGDMSIVFKHKPNGFG | Escherichia |
| | | | | ICYNLYTQYEPGPNLDYGALVNCMIELNLQAETLLFKP | phage ECO4 |
| | | | | VIYIPRIGCGIAGGDWDKVSKLIDMFTPDIDLIVVDYES | |
| | | | | TLPTSV | |
| tk.2 | YP_ | 61 | 119 | MSLSKEQKDKLFELIHELLDEHTEANTFYDEYGPLSPD | protein |
| | 009102582 | | | QQEEFADRFDKKENELIAYVNTL | ECML134_107- |
| | | | | | Escherichia |
| | | | | | phage ECML- |
| | | | | | 134 |
| DNA | QCQ57104 | 137 | 120 | MTRINLTLVSELADQHLMAEYRELPRVFGAVRKHVQ | endonuclease- |
| glyco- | | | | NGKCVKDFKISPTFILGTGHVTFFYDKLEFLRLRQIELIA | Escherichia |
| sylase | | | | ECLKRGFKIKDTTVQDISDIPAEFRNNYVPDEASIAISQ | phage EcNP1 |
| | | | | ACLDEKIAQRPTWYKYYGKSIY | |
| RB27_120 | YP_ | 92 | 121 | MAQLSAGFGYEYYTAPRRVSAAPKKIQSLDDFQEVIR | protein |
| | 009102325 | | | NAFQDYARYLKEDSQDCLEEDEI | RB27_120- |
| | | | | AYYTQRLEQLKNLHEVRAEVSKSMNKLIRFKE | Entero- |
| | | | | | bacteria |
| | | | | | phage RB27 |
| KRT47_ | QHB43119 | 137 | 122 | MKQLIIKRLNLLICCLCVVVAYGYYAINDYMHYKDYDV | protein |
| gp91 | | | | TVVNTLIGTQGKGSSLSFIAVYELKDGYRFSEYISPEMY | KRT47_gp91- |
| | | | | SSIEKGDNITVSLRPFDVKQTLFDNIVWFFGMVLVQSV | Shigella |
| | | | | CGAYICCYIIFRLAKLIETEIE | virus KRT47 |
| teqdroes_ | QHR64409 | 148 | 123 | MIINENSWHVRMHDFFYWQRPNSLCKYFWKMAWT | protein |
| 2 | | | | FLVIGVICASGLLGSWVVGQAILSQFGVTGFWLLHAG | teqdroes_2- |
| | | | | GTVFGVLIFATIVVIAVGIAYIVAKISGLWERVRYDRAW | Escherichia |
| | | | | KRQQDEENGIEHPKSVIVEYLKASKSKICPMIEFKDVK | phage |
| | | | | | teqdroes |
| AVU04_ | YP_ | 105 | 124 | MKTYQERIAESAAGQRVEGGYIWIEQVEPPSRTSTGK | unnamed |
| gp227 | 009210312 | | | WKITVDGPGGKRRTLKEINGYYEEALSDAGTYMMQI | protein |
| | | | | MIKNPENRFAARVFRRIGNKLYDAYSKNFPKE | product- |
| | | | | | Escherichia |
| | | | | | phage slur02 |
| N/A | VEV88946 | 102 | 125 | MCLSINNWDNFITYIKYFYYSNERKHSLKWLKNCNAII | Phage |
| | | | | TNACDQCDQNDETVLNVSKCYEEGDVVSIRQIDDERS | protein- |
| | | | | HIITFTKDEAIALKTYLDSVIPTMISK | Yersinia |
| | | | | | phage fPS-90 |
| BN81_ | YP_ | 70 | 126 | MLQLTEKQLRNLTVLQLDEIRREVGNVISALRREVSLN | protein of |
| 113 | 009149352 | | | QSPADYTRLRNFEKYLDKVKAV | unknown |
| | | | | HRHKVNTGQK | function- |
| | | | | | Yersinia |
| | | | | | phage phiD1 |
| Sf24_ | YP_ | 56 | 127 | MIYDISVSRTPSMIVTIPAEELDRLQKIEELLWEIESDLP | protein |
| gp210 | 009619290 | | | SGLESWIDYEELNKLRN | Sf24_gp210- |
| | | | | | Shigella |
| | | | | | phage Sf24 |
| phiC120_ | ARM70944 | 93 | 128 | MKTYSEFLSESILNEATDTFATKLGAALVEAESLLARISE | protein |
| c239 | | | | LASKIDTRKFERTSDIVKLEALL | phiC120_c239- |
| | | | | RMCDKPEEIAKNGSLMIKQRLEKYISGASEK | Escherichia |
| | | | | | phage phiC120 |

TABLE 5
Essential T4 Genes
Sequences are publicly available in
Uniprot, for example, as skilled addressee will know.

| | Function of | | |
|---|---|---|---|
| Geneª | gene product | Size (kDa)^{b} | Mutant phenotype |
| 56 | dCTPase; | 20.4 | Little DNA synthesis; |
| | dUTPase; dCDPase; | | unstable DNA |
| | dUDPase | | |
| 41 | Replicative and | 53.6 | DNA arrest; little |
| | recombination | | DNA displacement |
| | DNA helicase; | | synthesis |
| | GTPase; | | |
| | ATPase; dGTPase; | | |
| | dATPase | | |
| 42 | dCMP | 28.5 | Little or no |
| | hydroxymethylase | | DNA synthesis |
| 43 | DNA polymerase | 103.6 | No DNA synthesis |
| 62 | Clamp-loader subunit | 21.4 | No DNA synthesis |
| 44 | Clamp-loader subunit | 35.8 | No DNA synthesis |
| 45 | Processivity | 24.9 | No DNA synthesis; |
| | enhancing sliding | | no late transcription |
| | clamp of DNA | | |
| | polymerase; and | | |
| | mobile enhancer | | |
| | of late promoters | | |
| 55 | σ factor recognizing | 21.5 | No late transcription |
| | late T4 promoters | | |
| 49 | Recombination | 18.1 | No resolution of |
| | endonuclease | | recombination |
| | VII | | junctions; |
| | | | incomplete packaging |
| | | | of DNA; reduced |
| | | | heteroduplex repair, |
| | | | reduced DNA |
| | | | synthesis |
| e | Soluble lysozyme | 18.7 | No cell lysis |
| 57A | Chaperone of | 8.7 | Defective tail |
| | long and short tail | | fiber assembly |
| | fiber assembly | | |
| 1 | dNMP kinase | 27.3 | No DNA synthesis |
| 3 | Head-proximal | 19.7 | Unstable tails |
| | tip of tail tube | | |
| 2 = 64 | Protein protecting | 31.6 | Noninfectious particles |
| | DNA ends | | with filled heads |
| 4 = 50 = | Head completion | 17.6 | Noninfectious particles |
| 65 | protein | | with filled beads |
| | | | but tails attached at |
| | | | wrong angles |
| 53 | Base plate wedge | 23.0 | Defective tails |
| | component | | |
| 5 | Base plate | 63.1 | Defective tails |
| | lysozyme; hub | (processed | |
| | component | to 44 & 19) | |
| 6 | Base plate | 74.4 | Defective tails; permit |
| | wedge component | | plating of fiberless |
| | | | phage |
| 7 | Base plate wedge | 119.2 | Defective tails; permit |
| | component | | plating of fiberless |
| | | | phage |
| 8 | Base plate wedge | 38.0 | Defective tails |
| | component | | |
| 9 | Base plate wedge | 31.0 | No attachment of |
| | component, tail fiber | | tail fibers |
| | socket, trigger for tail | | |
| | sheath contraction | | |
| 10 | Base plate wedge | 66.2 | Defective tails |
| | component, tail pin | | |
| 11 | Base plate | 23.7 | Defective tails |
| | wedge component, | | |
| | tail pin, interface | | |
| | with short tail | | |
| | fibers, gp12 | | |
| 12 | Short tail fibers | 56.2 | Defective tails |
| 13 | Head completion | 34.7 | Inactive, but filled |
| | | | heads |
| 14 | Head completion | 29.6 | Inactive, but filled |
| | | | heads |
| 15 | Proximal tail | 31.6 | Defective tails |
| | sheath stabilizer, | | |
| | connector to gp3 | | |
| | and/or gp19 | | |
| 17 | Terminase subunit | 69.8 | Empty heads |
| | with nuclease and | | |
| | ATPase activity; | | |
| | binds single- | | |
| | stranded DNA, | | |
| | gp16 and gp20 | | |
| 18 | Tail sheath monomer | 71.3 | Defective tails |
| 19 | Tail tube monomer | 18.5 | Defective tails |
| 20 | Portal vertex protein | 61.0 | Polyheads |
| | of the head | | |
| pip = 67 | Prohead core | 9.1 | Defective heads |
| | protein; precursor | (processed | |
| | to internal peptides | to small | |
| | | peptides) | |
| 68 | Prohead core protein | 15.9 | Isometric heads |
| 21 | Prohead core protein | 23.3 | No or defective heads |
| | and protease | (processed | |
| | | to small | |
| | | peptides) | |
| 22 | Prohead core | 29.9 | No or faulty heads |
| | protein; precursor | (processed | |
| | to internal peptides | to small | |
| | | peptides) | |
| 23 | Precursor of major | 56.0 | No or faulty heads |
| | head subunit | (processed | |
| | | to 48.7 | |
| | | or 43) | |
| 25 | Base plate wedge | 15.1 | Defective tails |
| | subunit | | |
| 26 | Base plate hub | 23.9 | Defective tails |
| | subunit | | |
| 51 | Base plate hub | 29.3 | Defective tails |
| | assembly catalyst? | | |
| 27 | Base plate hub | 44.5 | Defective tails; |
| | subunit | | permit plating of |
| | | | fiberless phage |
| 28 | Base plate distal | 20.1 | Defective tails |
| | bub subunit | | |
| 29 | Base plate hub, | 64.4 | Defective tails |
| | determinant of | | |
| | tail length | | |
| 48 | Base plate, tail | 39.7 | Defective tails |
| | tube associated | | |
| 54 | Base plate-tail | 35.0 | Defective tails |
| | tube initiator | | |
| 30 = lig | DNA ligase | 55.3 | DNA arrest, |
| | | | hyperrecombination |
| 31 | Co-chaperonine | 12.1 | Head assembly, T4 |
| | for GroEL | | topoisomerase is |
| | | | defective |
| 32 | ssDNA binding | 33.5 | DNA arrest |
| | protein | | |
| 33 | Protein connecting | 12.8 | No late RNA synthesis |
| | gp45 and gp55, to | | |
| | allow transcription | | |
| | by RNA polymerase | | |
| | from late promoters | | |
| 34 | Proximal tail fiber | 140.4 | Fiberless particles |
| | subunit | | |
| 35 | Tail fiber hinge | 40.1 | Fiberless particles |
| 36 | Small distal tail | 23.3 | Fiberless particles |
| | fiber subunit | | |
| 37 | Large distal tail | 109.2 | Fiberless particles, |
| | fiber subunit | | host range |
| 38 | Assembly catalyst | 22.3 | Fiberless particles |
| | of distal tail fiber | | |
| t = rV = | Holin, inner membrane | 25.2 | Affect lysis by e |
| stII | pore protein, affects | | lysozyme; suppress |
| | lysis timing and | | T4 rII and 63 mutations |
| | inhibition | | |

^{a}Genes are listed by the currently used names, followed by alternative designations in the literature.
^{b}Gene products processed into smaller peptides are indicated (*) with the sizes or size range following the principal product.

TABLE 6

Tevenvirinae Phage
Acinetobacter virus 133
Aeromonas virus 65
Aeromonas virus Ach1
Dhakavirus
Escherichia virus Bp7
Escherichia virus IME08
Escherichia virus JS10
Escherichia virus JS98
Escherichia virus MX01
Escherichia virus QL01
Escherichia virus VR5
Escherichia virus WG01
Escherichia phage RB16
Escherichia phage RB32
Escherichia virus RB43
Enterobacteria phage RB43-GVA
Gaprivervirus
Escherichia virus VR20
Escherichia virus VR25
Escherichia virus VR26
Escherichia virus VR7
Shigella virus SP18
Gelderlandvirus
Salmonella virus Melville
Salmonella virus S16
Salmonella virus STML 198
Salmonella virus STP4a
Jiaodavirus
Klebsiella virus JD18
Klebsiella virus PKO111
Karamvirus
Enterobacter virus PG7
Escherichia virus CC31
Krischvirus
Escherichia virus ECD7
Escherichia virus GEC3S
Escherichia virus JSE
Escherichia virus phil
Escherichia virus RB49
Moonvirus
Citrobacter virus CF1
Citrobacter virus Merlin
Citrobacter virus Moon
Mosigvirus
Escherichia virus APCEc01
Escherichia virus HP3
Escherichia virus HX01
Escherichia virus JS09
Escherichia virus O157tp3
Escherichia virus O157tp6
Escherichia virus PhAPEC2
Escherichia virus RB69
Escherichia virus ST0
Shigella virus SHSML521
Shigella virus UTAM
Schizotequatrovirus
Vibrio virus KVP40
Vibrio virus nt1
Vibrio virus ValKK3
Slopekvirus
Enterobacter virus Eap3
Klebsiella virus KP15
Klebsiella virus KP27
Klebsiella virus Matisse
Klebsiella virus Miro
Klebsiella virus PMBT1
Enterobacteria phage T4
Tequatrovirus
Enterobacteria phage T4 sensu lato
Escherichia virus AR1
Escherichia virus C40
Escherichia virus CF2
Escherichia virus E112
Escherichia virus ECML134
Escherichia virus HY01
Escherichia virus HY03
Escherichia virus Ime09
Escherichia virus RB14
Escherichia virus RB3
Escherichia virus slur03
Escherichia virus slur04
Escherichia virus SV14
Escherichia virus T4
Shigella virus Pss1
Shigella virus Sf21
Shigella virus Sf22
Shigella virus Sf24
Shigella virus SHBML501
Shigella virus Shf12
Vibrio phage nt-1 sensu lato
Yersinia virus D1
Yersinia virus PST
unclassified Tevenvirinae
Acinetobacter phage AbTZA1
Acinetobacter phage Ac42
Acinetobacter phage Acj61
Acinetobacter phage Acj9
Acinetobacter phage AM101
Acinetobacter phage Henu6
Acinetobacter phage KARL-1
Acinetobacter phage vB_AbaM_PhT2
Acinetobacter phage vB_AbaP_Abraxas
Acinetobacter phage vB_ApiM_fHyAci03
Acinetobacter phage ZZ1
Aeromonas phage 65.2
Aeromonas phage Ah1
Aeromonas phage AS-sw
Aeromonas phage AS-szw
Aeromonas phage AS-yj
Aeromonas phage AS-zj
Aeromonas phage AsFcp_1
Aeromonas phage AsFcp_2
Aeromonas phage AsFcp_4
Aeromonas phage Assk
Aeromonas phage Asswx_1
Aeromonas phage AsSzw2
Aeromonas phage Aswh_1
Aeromonas phage Aszh-1
Aeromonas phage CC2
Aeromonas phage phiAS5
Aeromonas phage PX29
Buttiauxella phage vb_ButM_GuL6
Citrobacter phage IME-CF2
Citrobacter phage Margaery
Citrobacter phage Maroon
Citrobacter phage Miller
Citrobacter phage vB_CfrM_CfP1
Cronobacter phage S13
Cronobacter phage vB_CsaM_GAP161
Cronobacter phage vB_CsaM_leB
Cronobacter phage vB_CsaM_leE
Cronobacter phage vB_CsaM_leN
Edwardsiella phage PEi20
Edwardsiella phage PEi26
Enterobacter phage EBPL
Enterobacter phage EC-F1
Enterobacter phage EC-F2
Enterobacter phage EC-W1
Enterobacter phage EC-W2
Enterobacter phage vB_EclM_CIP9
Erwinia phage Cronus
Escherichia phage Lw1
Escherichia phage RDN37
Klebsiella phage AmPh_EK29
Klebsiella phage E1
Klebsiella phage KOX11
Klebsiella phage KOX8
Klebsiella phage KPN1
Klebsiella phage Marfa
Klebsiella phage PhiKpNIH-6
Klebsiella phage vB_Kpn_F48
Klebsiella phage vB_Kpn_P545
Klebsiella phage vB_KpnM_Potts1
Morganella phage vB_MmoM_MP1
Panteoa phage Phynn
Pectobacterium bacteriophage PM2
Proteus phage phiP4-3
Proteus phage PM2
Proteus phage vB_PmiM_Pm5461
Pseudomonas phage Psp YZU05
Serratia phage Muldoon
Serratia phage PS2
Shewanella phage Thanatos-1
Shewanella phage Thanatos-2
Shigella phage vB_SdyM_006
Sinorhizobium phage vB_SmelM_phiM10
Sinorhizobium phage vB_SmelM_phiM14
Vibrio phage vB_VmeM-32
Yersinia phage JC221

TABLE 7
Genes Between pin & iPII in T4 Genome
(Genes Permissive for Deletion)

| Gene name | Start | Length (AA) | Function |
|---|---|---|---|
| 49.1 | 47521 | 51 | conserved protein of |
| | | | unknown function |
| 49.2 | 47826 | 106 | hypothetical protein |
| 49.3 | 48131 | 102 | hypothetical protein |
| nrdC | 48391 | 87 | thioredoxin |
| nrdC.1 | 48635 | 80 | conserved hypothetical protein |
| nrdC.2 | 48936 | 104 | conserved hypothetical protein |
| nrdC.3 | 49859 | 308 | conserved hypothetical protein |
| nrdC.4 | 50915 | 333 | conserved hypothetical protein |
| ordC.5 | 51995 | 340 | conserved hypothetical protein |
| nrdC.6 | 52893 | 275 | conserved hypothetical protein |
| nrdC.7 | 53302 | 133 | conserved hypothetical protein |
| nrdC.8 | 53885 | 175 | conserved hypothetical protein |
| nrdC.9 | 54248 | 100 | conserved hypothetical protein |
| nrdC.10 | 55320 | 325 | conserved hypothetical protein |
| nrdC.11 | 56445 | 336 | hypothetical protein |
| mobD | 57208 | 259 | homing endonuclease |
| mobD.1 | 57828 | 181 | conserved hypothetical protein |
| mobD.2 | 57932 | 34 | conserved hypothetical protein |
| mobD.2a | 58165 | 38 | hypothetical protein |
| mobD.3 | 58349 | 64 | hypothetical protein |
| mobD.4 | 58534 | 60 | hypothetical protein |
| mobD.5 | 58722 | 62 | hypothetical protein |
| rI.-1 | 59205 | 128 | hypothetical protein |
| rI | 59495 | 97 | lysis inhibition regulator, |
| | | | membrane protein |
| rI.1 | 59720 | 70 | possible lysis inhibition |
| | | | component |
| tk | 60344 | 193 | thymidine kinase |
| tk.1 | 60534 | 62 | conserved hypothetical protein |
| tk.2 | 60716 | 61 | conserved hypothetical protein |
| tk.3 | 60925 | 70 | conserved hypothetical protein |
| tk.4 | 61389 | 155 | hypothetical protein, contains Alpp |
| | | | phosphatase motif |
| vs | 61733 | 115 | valyl-tRNA synthetase modifier |
| vs.1 | 62271 | 181 | conserved hypothetical protein |
| regB | 62740 | 153 | RegB site-specific RNA |
| | | | endonuclease, T4 mRNA |
| | | | processing |
| vs.3 | 63078 | 92 | conserved hypothetical protein |
| vs.4 | 63344 | 88 | conserved hypothetical protein |
| vs.5 | 63557 | 58 | conserved hypothetical protein |
| vs.6 | 63919 | 120 | conserved hypothetical protein |
| vs.7 | 64256 | 109 | conserved hypothetical protein |
| vs.8 | 64912 | 219 | conserved hypothetical protein |
| den V | 65355 | 138 | DenV endonuclease V, |
| | | | N-glycosylase UV repair enzyme |

Genes with known functions are in bold face. Homologues and orthologues of these genes perform the same function as shown. Sequences are publicly available in Uniprot, for example, as skilled addressee will know.

TABLE 8
(a) Elements of Recombination Donor Plasmids

| | UHS | | DHS |
|---|---|---|---|
| Plasmid | coordinates | Cargo | coordinates |
| pSNP898 | 1887-2625 | promoter-3 spacer array | 8092-8983 |
| pSNP902 | 1904-2668 | promoter-3 spacer array | 7178-8113 |
| pSNP940 | 7844-8643 | promoter-Cas3 to CasE | 10313-11117 |
| pSNP948 | 8873-9480 | promoter-Cas3 to CasE | 12224-12826 |
| pSNP951 | 1887-2625 | promoter-3 spacer array | 8092-8983 |
| pSNP958 | 1887-2625 | promoter-3 spacer array | 8092-8983 |
| pSNP996 | 8454-9067 | promoter-Cas3 to | 16673-17479 |
| | | CasE-2 spacer array | |

Coordinates of the UHS and DHS are the distances from the end of the
pin (protease inhibitor) gene towards the mobD and iPII genes of T4.
All the coordinates provided are with reference to the wild-type T4
phage genome (Accession number NC_000866.4 the sequence of
which is incorporated herein by reference and is SEQ ID NO: 129
herein).
(b) Phage Genome DNA Addition & Deletion Sizes

| | Added (X)* | Removed (Y)* | Y/X (%) |
|---|---|---|---|
| Phage 1 | 8365 | 8501 | 102 |
| Phage 2 | 7372 | 7799 | 106 |
| Phage 3 | 7695 | 6259 | 81 |
| Phage 4 | 7292 | 3553 | 49 |
| Phage 5 | 7629 | 4217 | 55 |

*Numbers relate to base pairs (bp) of DNA added or removed to
produce Phages 1-3. The column marked “Y/X(%)” shows that for the
listed phages, Y was at least 49% of X (in these examples Y was from
49 to 106% of X). Where X comprised nucleotide sequences encoding
a CRISPR array and Cas, Y was at least 50% of X (in these examples
Y was from 55 to 106% of X).
(c)Phage Genomes Sizes

| | Genome | | Proportion of |
|---|---|---|---|
| | size of | | Modified to |
| | Unmodified | | Unmodified |
| Modified Phage | Phage (bp) | Net bp Added | Phage Genome |
| Phage 1 | 167094 | −136 | 99.9% |
| Phage 2 | 169621 | −427 | 99.7% |
| Phage 3 | 168870 | 1436 | 100.9% |
| Phage 4 | 148612 | 3739 | 102.5% |
| Phage 5 | 148612 | 3412 | 102.3% |

Unmodified phage is the starting T-even phage (Phages 1-3) or Phi92 phage (Phages 4, 5) before removal or addition of DNA. “Net bp Added” is the net amount of DNA added to the T-even phage genome (a negative figure indicates that the final, ie, “modified”, phage has a genome size that is smaller than the starting, unmodified, phage, ie, more DNA was removed than was added). “Proportion of Modified to Unmodified Phage Genome” is the relative size of the genome of the modified phage to the unmodified phage.

TABLE 9
DPR Genes replaced by the CRISPR system components in Phi92.

| Gene name | Start | Length (AA) | Function |
|---|---|---|---|
| Gene 39 | 13672 | 184 | hypothetical protein |
| Gene 40 | 13880 | 50 | hypothetical protein |
| Gene 41 | 14339 | 203 | conserved hypothetical |
| | | | protein |
| Gene 42 | 14463 | 40 | hypothetical protein |
| Gene 43 | 15452 | 329 | putative site-specific |
| | | | DNA-methylase |
| Gene 44 | 15865 | 137 | hypothetical protein |
| Gene 45 | 16205 | 85 | hypothetical protein |
| Gene 46 | 16720 | 172 | hypothetical protein |
| Gene 235 | 141705 | 56 | hypothetical protein |
| Gene 236 | 141962 | 119 | hypothetical protein |
| Gene 237 | 142336 | 45 | hypothetical protein |
| Gene 238 | 142591 | 60 | hypothetical protein |
| Gene 239 | 142782 | 133 | hypothetical protein |
| Gene 240 | 143220 | 100 | hypothetical protein |

TABLE 10
Elements of Recombination Donor Plasmids

| | | Genes replaced in |
|---|---|---|
| Plasmid | Cargo | Phi92 |
| pSNP761 | promoter-Cas3 to CasE | 39 to 46 |
| pSNP762 | promoter-Cas3 to CasE | 39 to 46 |
| pSNP763 | promoter-Cas3 to CasE | 39 to 46 |
| pSNP776 | promoter-5-spacer array | 235 to 240 |
| pSNP778 | promoter-5-spacer array | 235 to 240 |
| pSNP875 | promoter-3-spacer array | 235 to 240 |

Plasmids were used as templates for PCR amplification of the sequence to be inserted. The primers listed for each plasmid determine the site of recombination in the phage genome. Plasmids were assembled from PCR fragments by InFusion HD ™ cloning and were sequence verified (Eurofins Genomics).

TABLE 11
>NC_000866.4 Enterobacteria phage T4, complete genome (SEQ ID NO: 129)

AATTTTCCTTATTAGGCCGCAAGGGCCTTCATAGTTTTAGCGATTTGGGAAACTTCATCATCACTTAAAG
AGTTGCGATAACCGATGAAGTCGGAAACAATACGGAATTTCTTGGTAAACTCAGCAACCATTTTATCACT
GTTTTTTGAAGCATTATTTGATAATACATCAAAAAGATTAGTTACTGTCCAAATGTCATGACCGATGGTA
TCTTTTCCACCATTAAAATATACACCCTGTAATGAACTAACCATATTAGCGAGTCGTGTATATTCTTCAG
AAACTTCATCTATACTGAAGTACTTCATCATAAAATCTAACTCAGGATACTTGATAATTTTATCAATATA
TCGTTTAGCTGAACTTGAATAACCTACATACTTATCATAATCTACATCATCAAAAGCATCTACATATAAA
TCACGCAAAGCTTCAAAAATACATTGGCACTGACCGAGTTCTTTTACCTTTTTCTGTAAAAGCGGACGAA
TAACATAAAATTCATTAATGCCAATAAGATTAGCCATACGAATCAAAATATTCATAGATGGATGACAAAG
AGATGTAGTACCATCCATAGAGAAAATATCAGAACGATGCATATACGCTACATAACCAGTAATTTCATCT
GCTTCTGATGTGAGGCGTAAATAATTCCTCTTTTCCCAGCGCCCGTCTTTAATTTCAAACTTAAACGCTG
TAGCAGCTTTAGGACGAGGAGCTTTACTTTTAACTACCTTTGGAATATAACTTTTTACTAAAGCTTCAAT
TTCTGACAAATAATGAATGTTAACTTCATCACTTTCAAACATCGCCATAATATCAGGAAGCAAATCAATC
TGCGATTCTACTTCTGGATTAATAAACAGAAGACGTTCGTTATGATGAATATTCAAAGTGTTATTAAATT
CACTATCATCTAACGCACGTGCTAATCCACGGACAATATTAACACGATTTTTAATATTATCAATAACGAT
ATTAATTTTTGTTGTATTAATACCAAACAGACGATAACTTGATGCAACGGCTGAAGTTTCATGACTTTGC
TTAATGCGCTTCAGTCGAGGGTCAAGATTTACTTCATACACAACTCCCGCGTTGCATAACTTACTGTCAG
GTTCAAACATGCTCTGCATCTTTTTATATGACAGATTTTTAGTCGTGAATTTGACTGAATTACTAATCAT
ATAATCTCGAGCAGAATACCCCATCTTCATCAATTCACGATATGTGTGACGAGGAGATGTAGATTCTTTA
AATCGTTTTACATCTTCATTAAATGCTTTCTCACTGAGTTCTTTAACTCGTTCAATAATATTTTTACGAG
TGCGATCATCCAGTGAAAGAGCCTCGCGAGATGGAGCAATATCAAGTGAACCCATTGGAAACTTAATGTA
ATTCACTTCATTGCGAATGCTTAGCCAGTTACGGTCTCTAATAACACCATCGATAGGATAAACAATACCA
CCGTAGATAGCATATAATCCACCACGATCAGGCCAGTATCTTTCTGGATTTACACCGTAATAGTCATCAA
AATCCGGAAAATAATCAATTTCGCGGTCAAGACCATTAATGATAGCCAAATCTTTGAACGGTCGCATGAT
ATAAGAAACTTCATAAGCAAAGTTTCTAAAGTCTTTTTCTTCAACTGGAACTACGATTTCAATACCAGTT
TTATCATCTGGACCCATTTCTTTTACGAATGTAGGTTTAATCTGTGGACCATCACCATCCATGTAAGCTA
CATAACCACGAATTTCACCTTTATGATACGAAGTAATACTAAACGTATCAGTATAACTAAACGGAGATTT
AGAACCTAAACCAAATCCGCCAATAAAGTCATTAGATTCAGCTTTAGATGAACTGAAGTATGAATTATAC
AACCCAGGAGAATTATCATCACCTTGAATATCAAAATCACTCATACCCGGACCAAAATCTCGACAAACAA
ATCGTGGGTCTAAACGTCCAGGAACTTGTATGATAAATTTTTCAGGATTTCCATTAAGTGCATGAGCATC
AATCATGTTAGTAATCAATTCACGGACTACTGCGCGAATCTTGTTTGTATACAAATCAGATGACAGAATT
TTAAATACTTTAGGAGATGCTGTGATGCTAAATGCTTTTGATTTAGAACCATTACCAAGAATTGTTTCTT
TTTCAGTGGTGATAATCATAATTTCCTCATTAATTCATATTACGCTTAATAACTTCAGCAACTTCTAGTA
GTTCATCTTTAGTTGCGGTGTCGGATTGAATTTTATCTCTAATATCTTTAAAGCGGGTTTTAAATTCTTC
GGCTTCTCCCATATCGAAAAAGCGTTGAATGATTCTATATTCTCGATGAACTGCTTTATCAAAAAGTTCT
AAATTTACTTTATATGATTTCATTTCAATATCCTCATTTGCCCAATTAATTATACCACATCCTTGTGGTA
AAGTAAACTACTGGCTCATCCATTCTTTACGAAGGTCAGCATTATCTCCCATGAGCATTTCAAAAAGCTC
TTTCCAGTTCTCAGGAAGTTTAACAACATCATATACTGGGTTTTGAATCATCTCACGATATTCAGATTTT
TCCAAAGAGCCAAGTCCCTTAATATAACGGATGCTATGTTTAGGTAGAGCATCTTTGGCACTCTCATATT
CAGCGACTGTATAAAACCATTCTTGTTTTTTACCGACCTGAGCGATGATTACAGGAGTTTTGACAAAGCG
AATTCGTCCTTGCTCAAACAATTCTGGCCAATTACTAAAAAATCCGAGCAGAGAAGGATAAATAGAACCT
AATCCAATAATCTCTTAATTATGAGGTATTTCTATAGATAGCCCGAAGGCTATCCATCGTGATCTGCGTC
TGTCATAATAGCGACATTCGCATAGTTCATTGAAGAGGATTTAATAGAACGACGAACATTGTTCTGCAAT
TTATATTTTTTCATATCAACGCTAGAAGAATCAATTTTTACAAATTTCATTATACACCTCATAGAACTTT
TCATCAGGAATCCAACCGCGTTTAAATTCATTAAATGCTCGGCCGAATAATTTTGAATTCACAGTTATAT
TATTAACTGATTTCCATTTAGCAACTCCCGTTCGTTTATAATGATCGGGGTCATATTTCGTGACGTACCA
TTCATATAAATTTGGTATTAATTTTACAGCCTCTGGATTTGTCGCTGATTTATTATACCATGGTTTCGCT
TTTTCAAGTTCAGCTGCTTTTGCAGTAGCAGCAACAGTATTTCCAACACCAACTAATTTTTCAGTTTTAA
TGCGTGGATCATTAACATGAAGACGAAAAACTTCGCCCTTTTCATTTTTAAAGCATGTCATGCCTTTAAT
TCCAGGAAGCTTAACACCTTTATTAACACCGCATCATTCCTTTGGGTTAAATGATCCTTTAATTAATAAG
GCGCATTTACCCGATTTAACTACTTCTCATTCAACAACTTTATCTTTCATAACGTTTTTTGACCATTCAG
ATACTGCTCTTTGATGGCTAAATTCTAGCAATTTCACTATAATTTGCACTAGAACGTAAACTATTTTTCA
GTGTTTCAACATTCTATTCATCGCATATGCCATTTCACGATTACGATGAATTTTATATAGTAGAAAATAG
TGCTAAAAAGTGTTCACGAAAAGTCATGTTTCACCAAATTTTCGTTATCATCAGAACCTCCCATTGATCT
TGGAAGGATATGATGAATTTCACCCTTAAATTTAGAAACGCGGGTTTTACCCCGCACTATTAAGTCATTA
TAGATTTTTTCGTAATTCACCTACTGTTATCCATTTACCATTAATCTGTACTTCATCATTTTCATTTACG
ATAATTGTATCGCCATTTAGCTCGAAAGTAAACCACTCGCCATCTTCTTTTTCTTCAAACGCTTTTTCAC
CGAGAACTAGACCAGTGATTGCGCAAATATCAAATAGTTCTTTGTTTTTAAGCATATCTGCATAAGACAT
ACCCCAACTGTTGAGAACTTTACCACGCAATGGATAACCACCGTGAAGTTCTTTATCACGAACATCAATA
AGATATCCGATAGCCGAATCACCCTCAGTCAAGAAAAGAGTAGTATCAGCATCTTTACCGCAAAGATTCG
CTTTGATATGTTTATGAACCTTAGCTTTAGAAGCCTTTTTAGCTGCTTTAGTTTCTGCTGCTTTTTCTGC
CGCCAATTTACGAGCCAAAGCAGCTTCAATAATCGGCATTAGAATTGCTTCATTATTTAGAATATCACGT
GAAATCTTTTTAGCATCAAGTTGAATATGACTACGAATTTCGCCAAATGGAGAAGTCAAACGCTCTTTAG
TTTGACGAATCAATCGCATGTTTTTCATATCACGAACAAACATAACGATAGTCAAACATTCTTTGACACG
TGCTTTAGTCACATCAATTTTGAACTTACGTTTGATTTGTGGAATAAGGTCTTCACAAATATCATCCATA
GCGCAGTCAATGTGATGGCCACCATTCTTAGTATGAATGTTATTGACGTATGTTAATTGACGAAAACCAT
CCGGTGAACGACCAACCGCAATAGAACAATTTTCTTGCTCTTGAACAATAGCATGTTCATCATACTGCCG
TGCATATTTCTTAAAATTGCCCTGAACCTTTTTACCATTAAAGGTAAATTGAATATCAGGATAAACTACA
GCAAGTGTCTGGAGACGATCCAGTGTAATGTCAAGATAAACTTGGGACAGCTCATTAGTTTCAAATGACA
TAAAATCAGGAATGAAAGTAACACGAGTTCCTTTCCATTTTCCAGGAATATCTTCCCATGATTTATTTTC
CATGCCATTTGAACAACGAACTACAATATTATTTTGACCGTCGCCAGTTTCACCGACAAACATCACAGAA
AAAATGTTTGTCAAACTAGAACCAACACCGTTCATACCGCCGGTGACGCGTTCTTTATCATCACCAAAGT
TACCACCTGCTTTTGGAATAGTCCATGCGGCAACAGGACCAGGAATTTCTTCACCGGTAGGTGTTTTAAC
CATCGCTTGTGGAATACCGCGACCGTTATCTTCAACTGTTACTTGATTGTTTTTAATAGTAACATTAATT
TTATTCGCGAATTTAAACTTAGTACGAATACCTTCATCTACTGAGTTATCGATAATTTCATCAATAAGCT
TAACAAGACCAGGTACATACTGAACACTTTCCCATTTACCAAACATAAAGCGCTCATGCGTTTCATTAGC
AGAAGAGCCAATGTACATGCCACTACGCTTTTTGATATGTTCAATATCGCTCAGAATTTTAATTTCATTC
TTAATCATCACTTATCCTCGTTTGGTTTCGGGAATATTATACTCCGGTAATCATAAAGCTAAAGGCCCGA
AGGCCTTTTATTTAAAACGAATAGTTGAATCCTTAAAGAACAGCCCAGAACATACTGTTCCTTCTACTTT
CTGCCCGGTAGGTCCAATAGCACGAAATCCAGTATGCTGGAAATCATTTTCAGAGCAACCGAACCAATTA
TATCCAGTGATTTCAATATTAGTAAAACCACTTGAAGACAAAACTTTGGTTGCATTAATCAGCATCAGTA
CTATTAATTAAAGACACTGCTAATACTAATGCTGCAATTGAACGACTAATATATTTCATAACTACCCTTT
AAGCAAGTCGTAAAATCCATTATTCCCATGCTTAGGAAGCGGAAACTAACCGAACAGCCAGCCGATGACA
ATCAGGACATACACCAGTATCTCTTCCAGAAATTTTCTTGATTTTTTCGTATTCTTTTGCACAGTCTTTG
GATTGACATTTATAATCATAAAGCGGCATAATTATTCCTTAAAGTAAGCTTTCAACATCEGATATAAAGA
CCACGCCTGATCATTATTTTCAATAGTAACTTTCATGACTGGGAATTCTGTGAAATCTTCTATTTGTTCT
TGCTCTTTCTCTTCCTGCTCTTGCTCTTCAACCGCCTGATATGGATTTTCCACTTCATCAAAGAACCCAG
CTTCGTTAGTAGAGAGCCAGATAAAGTTTTCGTCAAGGATATCACCGCCGGCACAACGTTTGAGTACACC
TATGGATGTCATAATTTTAGTAGGACGCCCAAGATAATCAGCATCTAAAATTTTAAAAGGCTCCATACCT
AAACGTCGTGCATAGATTCCGTTATCAGTATGGTCTTTAATAAAATTTTCTTGAGCTTGTTTATTTTTAA
ATTGATACCATTTATTAACTTCAAATTTAATAGCCATTAATAAATTTCCTTCCAGTAAGTTGTGCCGTCT
TCAGTAATTTCACGAAATACACCATAAATTGGCTGTTTATCACCGACTTTCTCATACACATAAACAGAAG
TCAAGTGAGTAAACTTGCTAGTATGTTCCTTTTGAACTACTACCAAATTTGGATCAAATAATACATCTTC
AAATTCATCATTAGTGCAATTCTGAACAATTTTACGTTTCATTACAATTTCCTCATTAATTGAACAGTGG
AGCGATACGTTTCAGAAGAGTATCAACACCTTTAGCGAATTTTCCATTTTATTCTCCAAGTTGTTTTCTG
TATCAGTAGTTGATATTGATATAGTACCATAATCAACTACTGATGTATATAGTTTTATGAAAAATTTAAA
CTTTATGCATAGAGAGCATTGCTATAGTGTTTAATCCAACTTTCAGGAATGACTTTGTATGTTCCTAAAA
ATACCACGTTGTACAACTTAACACCATCTTCTACCCATTGATCGGTAATGTATCCACACATAGCGCGAGT
ATAAACAACCCTTCCATCATCTTTAATAAAGTTAAATTCACAAGGAGCAATGAACTTGATAGCCTGACCG
AGTTTCCACTTAAAGTCTACACCTACATGCGAAGTATCAATCGTTTCAATTCCTTTAGCAGGAACAGCTT
TTAAAAACGCAGACTCAAGAAATTTCGCACGAACATAGCCAAACTGGGGTTTAGACTTTCCATCTTTAGG
AATGATACGCACTTTTACTTCAGAATCTTCATCTTTAACACCATGCTTAAGCTGAATGCTTACAACTTCG
ACCAATTTTCCTGCTGCTTTAGAACGGGATTTATCAGATACACGAGCTAATTCACCAATATTAATAATCA
TAGTTATCTCTCACTTGTTAAAAAGATTTTATACTCCACGGGACCATTATACTCTGGTCCCAAGAGTTTG
TAAACTATTAATTCAAAATAGCTACCACTGCACTACGAGGAACTACGGAGTACTCTCCAGCATGAACTAC
GTTCAGAAGTTCAACGCCATCTTCCAATCCATTGGTCAGTTACCCAACCACCAATCGGATTCGCAAATGG
ACGACGAATGTAAACTGCCTTACACAACAAATCAGTCGGGTCTTCAGGTTTTTCAATTTCTGTCAATAGA
TTAAAATCTTCTTCATAGATGATGAATGATGATACCCTTCCATAATAGTTTCTAATTTCCATGTACACTG
TACCAATGAGAATTCCACTATTAACACTAATGACAGTAAAAGGATATCCGCCAGTTGTACCAAGAAGTTC
CCAAAATTTGCTATCAGTCGTATTCGACATTGTCTGAAAATCAATTTCAGATGGTTTTTTCATTTGATAC
GCAGTATTAATTTTAATCATAATTTTCTCTTTAGTTTAAGGTAATAAAGCCTTTTAGTTCGGCATAGGAT
TTACGGAACATTACTTGATGCCCGCCAATGATAACTTGGTCATCTGGTACTTCGTATACAGCAAGATAAA
ATCCTTTCGAAGCTAATTCTTCACGCTCTTCTCGTGTGAACCATTTCATCATATCATATTCGCTAGCAAA
AGCAAAATGATAAAGAGCTACAAACCATCCGGGAATATGATATTCTACTCCAACATAATCTTTCTTGAAC
TTAGTATTAATTACGATATTAGCATTTTTAACTAATAGTTTGTCTTCGTGCGGCACAGGAATTCTTTTAT
TATCATTACTATGATGCATAAAATTAGGTCTGTCATAACCTACATGTAATAACCACTCTTCACTCCATGA
ATCTATTATACTTCTATACGGCGTTATTTGAACACAAAGATCTCGGCGTATTGTTATAGCGTCTTCATAA
TTAAGAATACTAAACGATGATTCAACACGATAAATTTTCATTTTATTATCCTCAGTAGCTATGGTGTTAT
AGTACCACAACTAACCGAGGAAGTAAACAACTTTTTATCGTTTTGTTGGAAGAGATAGAGGATCGCATTC
TTCCTCTGATGGAGCATCTTCAAGACCCATAGCATATCGCAAAGCATACTTCATCATCAGGATGTCTTTC
GCACAGTCATGAATAGAATCATGTGCAACGAATCCATCTAAAGTTCCCTTTGGAAGAGGACACGTTGTCA
TATCACGAACAAGCAGAAGTGCTTCAATTCTAGTACGAATATCACGCTGATTCCAAAATTTACAAGGTTC
TAACTTAAATGTATCAAGCTCATTCTCGGAAACGCCGTTAAGACGTTGAATATCGCGAATAAGATCGACT
AAAATTGGAAAATCAAACGACATTCCACGGCACCAGCCTTGAGATTTCCAAGGATCGATATTATGTGCAT
TGATGTAATCATTAAATTTTGCAATACCGTCGATAGTGCTTACATCTTCATCGGATGGTGCAATATTTTT
TCGAGCTTCAGGAGATTGATTCTTCCACCATTCGATAGTACTTTTAGTAAAAAGACGGTGTCCTTTTTGG
CTTTTTAAATCAAATTTGATTTTAATGCCACGTGAAACTAATTCATCGAATGTTTCAACTACTTCTGGAT
TAGGGTCAAAAGCAATTACAGCCAAATCAATAACCGCTGCTTTTTCACCACTTCCCATTGTTTCAAAATC
TATAATAAAATCAAACATTAAATTTTCCTCGCTAAATCACGAATTTGACCTACAGTATAGTCTTGAATAT
AAACTTTATTAATAGGCTCATCAATAAATTTTGCCATAGATTCAATATCTTTTTGTATTTCTTCAAGACT
GTATACTATCTTTGAAGCTTTTTCGCGAATAGTGATATTTTCAGGACCCGGATTTTCTTGAATGACAACT
TTAACATTTGTCATAAGAGATTTAAACTGGTACCAACTTAATTCAATCATTAATAATCGCCTCATAAAGA
TAGCTAATTTCGCCTAAAACATAATCATTGATTGTAACAGTTTTAACTTCACCGCAAAAGAATTCTAACG
CAATTAAATCTCGTTCAATTTCTTCTAATTGAAGCATCAACTTACTAGATTCAATTTTTACAGTTTCACG
ATTTTTGCTATAAGCTATTTCATAAATTTCGCTTACTTTATCTTGAAGAAGATAAAACTGATCTTTAGTT
ATTTCCACGAATAGCTTCCTCAAATTTAATCATACATAAAACACATCATAACGACCACGGGTGACACCAA
CATAAAGAAGTTGTTGAGCTAATTCAACATCTGCATAATGAATACAAGGCGTATAAATGAAAGCACGGTC
TACAGACATACCCTGCGCTTTATGGAATGTTGATGCAGGAAGTGCTTTCACTTTACTAAACTGTGATTTA
GCATCCCAAAAATCACTCCACGGAGCTTTTCCGCCTTTGTTCCAATTTTTATAAGTTTCTGCTGTTTTAG
CTAAAAATAGGTTAAACTTATACAATTCTTCGTCAGATGAAATTATTTTAATCTTTTCACGATAATATTC
ATCATCGCCATAAGTTTCTACTGTTAAATCCCAATGACGAATTAGATATTCTCCAGGAACACCACGGGCT
TTAACAAACGTTGATGTATACTCTGCTTCTATAATACGAACTAATTGTCCGTTATTAAAAATAATTTCTG
ACACAGGCTTTCCATCAATTTTATATGTTTTAAATAATGGTTCCTGCATTACAATAATTTCACCGACAAT
AAAATCTTTATCAGTTTCAAAAATCTTTTTACGAATAATGCTATTTAACTTGTCAACAGATTTATTCGTA
AATGCCATTACGCGATTTTCAAACAAATCATCTAGTGATTTGACGATTGAAAAATAATTTACCATAAAAT
CGCGTAAAGCGGTATCACCAGTAAATCCACGTACTCCATGCCCGTCAACAACTTTATCATAATTCCACTT
ACCGTTGCGAACGTCAGTAGCTACATCAATAATAGGAGCATTACTGCGTTTAACTTCAGTGAGTTCACAC
TGATAAAAATCTTTATGTGTAAAGAATGGACTGATATAAGCAGTATTTTCTCCTGGTTCAACAGGTCTGA
TTTGCTTATTATCCCCTATTCCAATTATAGTACACCAAGGTGGAATAGTTGAAAGCAGAATTTTAAATAG
CTTTCTATCATACATTGACACTTCGTCGCAGATTAATACTCTGCATTTGGCTAAATCAGGTACTTCTTTT
TGTTCAAAAAGAACATTTTCTTCATATGTTACTGGGTTAATTTTAAGAATACTATGAATAGTACTCGCTT
CTTTCCCTGATAGTTTTGAAAGAATCTTTTTAGCTGCATGTGTAGGAGCTGCTAAAATAATACCAGTTCC
ACCCGTAGATATTAAAGCTTCAATGATGAACTTAGTAAGAGTAGTCTTACCGGTACCAGCAGGTCCATTA
ATAGTTACATGATGTTTCTTTTCTTTAATAGCCTTCATAACAATGTTAAAGGCATTTTTCTGGCCTTCGG
TCAAATCATCAAATGTCATCGTAAATTCCCTGCAATTGGTATACTAACAATACGCCCAGTATCTAAAATT
CGCTGATATAATCTTTGCGTGTCTACGTCAGGCTTAACATGTTTAACTTCTATTTTATTAAACCAAAATT
TACGTGGAGTCTCAACTAATCTTGGAATTCCCTTACCTAAAGCTAATCGATACTGCTCTTTAAGAGTGGT
AAATACTTTATCAGCAATCTTCCATTCAAAAAATACAGCAGGACGATGTTCATCAAGCGGAACTGGCGCT
GTAAATCCGTCTTTGTCTCGGTAAACTATCGCATATACATAAACCATATTATCCTCGGATAAGTTTAAAA
ATTGAACAATTTAGCGGATATCCTCTTTTCAGTTTAAGTTTATCAATAAAAGACAAATTTTGATACCGCT
CTACACCTTGAATAATTTTATCACACATATCATATTGCATTTCTGCTTCTGACAACTTTTTCACAATTTT
CCAATCCGAGCCTTTAAGAAGAACGTTCAATTTAACAACTTCAGCGCCTTCTGCTATGCGAGAACCATCA
ATACGTGCTTTAAGTGCTATAATTCTCAGCTTAATGTCAGAGGTCTGTTTTGATTTAGAAAGCTGAGAAA
TGTGTTCAATTCGATTTTCACGTTTTTTCTGTATAGCTTTAATTTGATTATAAGTCTTTTTGATTTTAGC
CCATTTCTTTTCATCTAAATTTAGTTTATGAACTTTTTTCGCAGATGAACGACCAATTCGCAAAGCAAAT
AAATCACGCTTTTCAATCAACTCTTCTAAAGTATAATCAGAACGAAATGTATTATACTTTTTCTTTACTG
CAATAACATTCCCTTTAATGTATCCAACGTTATTATCAAAACGTTCTAATGATAATTTCTCTCCTTCAAT
ACGATTATCAAAAGGTTCTCCCGAGTAAGCACAAACTTTTTGATCTAAAATGTTCTTAATGTAATTGAAG
TCTAAGTTAAAATCTTTAGAACGTCTTTTTGCAGATGCCTGAGTATGCTCTAAACGACGTTTAATTTTAC
GAATTTGGTTATTAGACAGCTTCATATTTTTCTCACATCTTACGGACGGTTAACTACTTATACTATAACA
TTTTTACTTTAACTTGTAAACAACTTTATGAAAAATGCTTTAAAACTTTCATGGTATAATGAATCTAAGT
CCTTCCATTATAGATTAAATCCTTCAAAATCAAGAGTATAGATAGTGTATGTTGAACACTTTTTATACTC
ATATCTATCTGCAATTCTAAATACACTTCCAGCTGGTATCATTACTTCTTGTTCATCTGAAACTAATTCC
ATATTACGATAACGATGACTATCCGGAAACTTAAAGTTTGGATTGTATTCTTTACAGCGTAGAGCTTTTA
TAGCATACTCCTGGAAATTGAATACCATAGGAGCTTTGAATTCAAAAATAACTTGTGTGTTATACTCTAA
ACCAGAAGCAAAATGTAGAGCTATATTTTTATCATATGAAGCTGATACGACTTTATCAAATGTAATAATA
TCAATTCCTTGATTTAATACTTGTTTAGTCTCAGCTGGAACACCTCTCCAAAGAGGTTTATCGTTTGGAA
CCAAACGAGATTTGATTATTTCATTTAACCAAGAATGGTCATCTGGTTTATTAGTAATACAATGAATTAA
AAGTTCAATTTCAGATAAATTAAACCCTTCAGAAAGTAATTCTTCACGAATAGAAGCACGCACCGATGCA
TCCATTGATTTTATTTTAAAATCTTTTAGTTGCATTACTGAGTATTTCATTCAACTACCTCAATATCATA
AACTTTAAATGTTCCAAATGAATCGTGTAATTTTTCTTTTGAAATAGAAGTTATTTTATACTTTCCAATT
GGAATCATCCATTCTTGTTCACGCACAATCATCATTAAGTTATCAGTACGCTCTGAATCTAATCCATCAG
TATCTTCATACGTGTACTTAAACTCAGTATTAGGAGAAGAAAGTATAATATCGCTGATATGGTCAGAATA
ATTAAAAGCTTTATCAGTTTTTAAACGAAGTATTGTTTCAGTGAAATATTCAGCATAAGAAAAAGAACAC
GCTGTATGCAAACTAGTAGTAAATGAATCTACCCTGTTCGTTGAAAACACTTCTCCAACTTGTAAATCTT
TAATGAGTTCTTTTGTCGATTTTGATATACCACGATATAATTGATAAGGCGATTTAGTTAAATGCTTTTT
AATGATTTCATTTAAATGCTTATGAAGAGCTTCATTCTTTTTGGCTTCCATACATTGCCAAAGAACAGAC
TGCTCAAAGTCAGTAAATTTTTCACAGACCTTTTTATACATATCATATTGAAAATCAACGCTTTCAGCTT
TTATAGATAACTGTTCAACATCTGCAAGATTAATAATCATGATAGCCTCCGTATACTTCAGAAGCTATCA
TATCATCGTTAGAAAGGAAAGTAAACAACTTTTTGAATTATTTTGCCCAGGGAGCCCAAGGCGGAGGGTC
AAGATGGTATGAAGCTAGTTCTTCTAGAAGAGCATCTGGGGCTTCAATTCCATAATTCTGTAATACTATA
CGGTACTCTTTCTTATAATCACTAGAATCATTCTGGTTATTCGTAGAATGATTATCTTCTAACATCTCAA
ATAAATCCATATTAATTCCTAGCGATAAAAACCAAATTTACGATTAGTTTCAATGATCTTTCTTTCTTCT
TCGGACATTCTCCAGCGTAGTCCAACATCAAAATGAGCCCAGACCATCCTAACAAATGCATTTATATCTT
TAATATCTTCAATAATGAATGTTTTACTTTTAGAAGGTTGACTCGCTAATTTAACTTTATCGTCTTCAAA
CATGTCAAAAAGACCATATTCATGAGCTCTTTTATAGCCTTTAATAGTTAAAGCTTCAGAAGAAGAAAAT
GGTGAATCTGTATTCTGCAAAATATCTTCAGTATGCTTTATAATTAGAATAATATTTTCTGGATATTTTC
TTTCTTTTATATCTTTAATTAAAAAATCCGGATTTTCTGCTAAAGGAATAATTAATGAGCAATTTTATTC
AAGTGTATTTACTGATTTACCTTCTTTAGACATAAATTCTATTGAATATAATTTTGCTACTTCAATCATG
TGATTTCCTTTTGCCTACTAATGGACCGTCAGGAATTTTATTTTCCTGGATATATTTCTCATTTTCTTCC
ATCATTTTACTGCCAATTTTAAGAAGCAAATCCATTGCTTCATTTGCTTTTGCTTTAGCTTCTTCTAACG
TCATATCTTTGTTCATGATTTATCACCATAGATGTCTCTCATCAATTTAAGCGCTGAGCGTTCTAGTTTC
TTTTCTTTCTCAGCACTAATCATTGATTTCATCCATTCTTCTGATTCATTCTGCATTTCTTTATTTGCTT
GTTCAACCCAACCGTCATCAATATACATTGAGTTTGGTCTATTGAACCATTCAAGCATCTTCTTCAGAAC
TTTCATTCGTTTTACCTAAAACAATAGTAGGAGCATCGTCAAATTTATGAATTTTTAGCAAATTTGGATT
TAAATTATTCCATAAAGAGGTAATAAAATATGATAGCGCACTTTCGTCCGTAATTATAAATTTATTTCCT
TTATCTATTTTCCAATCATATATTGAATCATATGAATAGAAGGATAACGGTTTATTATTATAATATGCTT
CAGCAACATCAATATAGTTAGATTTAGTAAATGCTTGAATTGCCATAAATGGAGAATTTTGTACAGTTTC
AATAATTCCGATCTTTTTAAGTTTTATTTCAACATCTTCTGGAATTGGCATTGAAATAAAATCTTCTACT
AGATACATATTATTGTCATATCTTTTATCAATCATTACTGCTACAGTAATTGGAACATCCTTGACAAACG
CCATACTAATACTATTGATAGACATTTCAAACAAAATTGCTTCCATAATTTTCCTCAATCACAAGATGTA
GATGAACAACTAGAATCACAAGAACTTCCACATGAATCACCTGCCCATACATGAACAGGAACATTAGTAT
CATATGAATCAGAACTAGACTGTGTATTCTGTGTGTTAGATGATGTAGTAGGTGTTGACCAGCGCCAAGG
ATTTTTATAATATTCTTGGGCTTCTTCATAAGTCATAGTAACTGCTTCTACTGTTCCATCTCCCATATAA
ACATATTCAACTACAGTTAAAGGAAGGTAGTCATTTGAAATAGGAACTACACCTTCCCCAGGAGTTGTAG
AGAAAAAATCCGTAAAGAAACTTTTAAACCAATTAAAGATAAACATTACAAAAAGCCTCTTTTGAATTCG
ACTTGCTTCTCACCATAATCATATCGAATCTCTACATTAAATTCGACAGAACCATCTGCGTACATCATAA
ATGAATGCACAACAACTTCTGTAGACCATGGTTGTAGTTCATATTTCTTCATTACATGTCGTGAAATGAT
AATATCTAAATCTTCATTTGGTTTAATCCAACGATTTAACATAGTACTCTCCTCTATAAGATAATTCTAT
TATACCATACTCATTTTGGAAAGTAAACCATTTAAATGAAAAAAGGACTCCCGAAGGAGTCCTTGAGTTA
TTAACCAGTTACTTTCCACAAATCTTCATTTGCAGCAATCCATTCAGTACGTTGATTTTCTTCATATACT
GTAGAATATGCTGCTTTTTCTGAAGGGAATGTCTGGTAATGAGCGCCAGAAATTTTGTGAACGTCAGAAT
AAAGAGGGAAAGCTACAGAAATTTCCTTTCCTTCAATTTTCTTATTTCCATCTAATTTCTGCTCAAATGT
TTTGATATTAACATAACCGCGAGTACTAGCCATGTAATTCTCCTCTATTTAAATTACATGATTATTTATA
CATCTTCTTTTCTGAATAAGTAAATTAAATTCTTAAGAGCCGAACTTGTTACATCATATTTTCCTTTAAG
CGCCTTTACAACCGGGCCTGTTGCTGGTTTACCTAAAGAAACCCATAACTCGTGTATTTCGCTTTTAAGT
GGTTCATGCCAATGCGGTGCTTTTCTTTGCGCCCTTGAAGTTCCTTCTGAAATCTTTTTATTTCCGCCAT
TCGAATAAAACTTTTTCATTACTTCAGATTGCCGAGCTTTTCTTTCTGCCCTATTTTGGGCTATACGTTG
TGAATTCTTCATCCGAGTTTTTGTTTCAGGATTGTTTAATCTAAGTTTATGTTCTAATCGTTGTTGCTCA
GTCCATTTTCTTCCTTCTCCACCCTGACCACCAGGAGAAATATTAATGCAAGTATCTGGGTGTTTACGTT
TTAATGCAGATATTAGCTCACGTTCAACTTCATATGATTTTTCTCTAGAACCATGGCATTTTGACCATCG
TATTTTATAATTAAATCCATACTTACGATATATGTTCCATAGTATTTTACCTGAACCCGGATATTTATCA
TTATATGGATTTACAATAAATGATTCATGTTTCCCAGCGTACCAAAAGACGCCTTTCGGCGTCCTAACTT
TTACTATATATGTTCTATAAAATTTTTGTTTAATATCCATTATCTTGACGTTCAAAATTATGTTTGTTCT
TCAGATAATAAAGTTTAAAGATTTCTTCCGCATTCATTCCAAGGCCAACAAACATATTTAATACGAAATG
AAATATATCCACAAGCTCAAATTTAATTTCGAGCTGGTCTTCGGGGGACATTTCATCAATGCGTTTTTCT
TGCGCTTCAATATAACGTGCTTTCCATTTTTTCCATACAGCAGAAGCTTCTTTTTCACCACGTGACATTT
CACCAAGAGAAGTCAGAAGTTCGCGGAATTCATCATCAATACAGTCTTTTTGTTCACGCATCCAAGAAAC
AACATCACCGGCAGTTTCTAATTTATCTGGATGATAGCAGTATTCGCGGACATTAGCCAAACGAATCTGT
AAAAACCGCTGCATATCAAGCATAACTTGCAGCGGATCTTTTTCATCACCGAGAATATCCCAGTATTCAT
TTTGAGCTTTATCAACACCTTCGATCAAATGAGCACATTCATTAAAGTGAGCCATTAGTTTTCCTTTCAA
TTCATTAATAAGTTAAATAATTATATCATTTGAGTATGTAAGCAATTAATTAAAAATATATACTTCATCA
GTTCCATTCTTTTCTTTGGAATGATATATGTTAAAGACGTATTTTTTATTAAGATGCTTTACATTATATT
TTTTAGACCATTCTTTAAGAAGAGTGTTTTCCTTTCCGTGGTGTTCTAAAACATTCGACTGCCCAAATTT
TATTCCTCTATCATTTAAAGAATCTAAAAGATTTAAAAGGTCTTTTTCTTCATCTTCTGACCAAAATTTA
TTATAATCAGCAACTGTTATGAGATACGGAGGATCTACATATACAAAATCGCCGTCTAAAATTTTAACAT
CTTTAAAATGCAATGAACTAAAGATTATTTTATCACAATTTTGTTTAAAGTGATTATATTGTTTTTCACT
ATTTTTGTTTATAGTTCTTTTTCCAAACGGAGTAGTAAAATTTCCTTTATCGTTTATACGAATCATATTA
CTAAATCCGTGAAAATGAAGAACATAAAGTAAAAGAGGATCTCTAGTTTTATTATAATCTTCACGTAATT
TCAAAAACTCTTCTTTTGATGTTTTTGATAGTTTGTATTGCTTTATTACTTTTAAAACGTCATCCCATGA
TACATTAATAAGACGCTTATACATTTCAATAATTGGTTCTTGAATATCATTGGCCAATACAGGGCCATTA
ACATTCAAAGACACTGATAAACCTCCACAAAATAAATCCACGAATCTGTTATATTTTGGAAAGTGAGATT
TGAGTTCAGGTAATAATGATTGTTTATTACCTGTATACGCGATAGCTCCTAGCATTATATTCTCTCATTT
ATTGCAGCAAAAATGAATTATACAATTCTTCATCATATGTATTTGATAGTAATACTAACACGTTTTGCGA
TAAATATTAATTTAAAGGAGGATATATATGGTACAAAAATTAATGGCACTTGTTAATGCCATAAAAGGTA
ATAAGAAACGTATAGCTTTTACTATTTCTACTATGGTAGGAATTTTACTCTGGAACTTTATTTTATCACC
TGTTGCAATTGCACATGGTGTTAATATTCCAGTAGTTACTCTTGATACATTCGTAGATTTAGCATTTGCT
TTAGTTGGGTTAATTTAAATCTTAGCATATTTAGATAGCCGCATTTTAGCCATTAACCCCTGGGCAATAT
TATTTTTCATATATTCCATAATTTGTTCAGGGGTTGCACCTTCCTTTCTAATCATATCATTAACATCTTT
TGATTTCCAGGGAGATTTATCCCAAAACATAACCCTTTCTCCTGCATCAACTAATTTAGTCATTCGTTTA
ATAGTGTCAGGGTGACGAGGTTCATTATCTAAGACCCACACACGTCTATCTTTAAATGGAACAACTTCTA
GGTCTAATTGACCGCCCGTAATAGCTATACCATTTTCAATAAAAAGTGAATCTATAGGTCCTTCTAGAAC
ATATACATCACCATCTTTAACTCGTTCGACTCCATAGATTTTTGTTGCCTCAGGATAAGCTTCGATGGTG
ATATATTTTTGAGGAGCATCTTTCTTTAATGCACGTCCTTGAAAAGACTCAGCTTTTCCATTAGCATTAT
AAATTGGAATAACAAGACGAGGCTCAGAAATTTCCTTTTTGTATGTTCCCGGTGCTATGCTATTAACTAA
TTTAGGCCATTCGGTTGTAAACCAAAGATATTTCCATTTATCCTTTGGAATACAACGAGCTTTTACGTAT
TTTATAATTGGATGGTCTTCCGCCAGTTTATCTAATCTAACACATGACGGAAGAGATTTAATTATTTTCT
TCTCGGGTTGTTTAGGAAGTTCTTTAGGTTTTTCTATTGGACGACTTTTACCTTTTTCTTTTCTTATTTC
AAAGATATACTCACGATATAAATCGGGTTCAAACTCCTTTAAATATATTCCGATTGGTGCATGATAGTTA
CAGTTATAACAATGAATATTTCCTTCATTATTATCACCATAATACCATCCACGGGCTTTATTCTGGTCGG
TTTTTGAATCTCCACAAACAGGGCATCTAAACCGTAATTTAAAAGTTGAACTATTATTTACTTGTGTGAA
TTTAGGTAAATGAGCTAATGCACGGTATGCAAACTCATTATCAATCCAAGGTATTGATGACATTTTTACT
CTTCTTTTTCTTTAGATTCCTCTTTTTTCTTTTTAGGAATCTGTTCAGGACCTTTATTTACTACAGCGCC
TGATGTTGTTCCAATAGAGATATTTTCAGGATTACCACCTGAATCTCCAGCGACCATATCTTCTTTGATA
AATTCTTTAAATGTTTTCATATTAACCTCTATTCATAAAAGCATTAAAAATTTGGTCATCAATAGAAACA
TTTACTTTAGGCTGTTTTTCAGATGGCAATTCATATCCACATATTACAATTTTATGATCAATATCAAAAT
ACACAGAAGCAATATGATTAATGATATTTTCAGTAAAGTCTAAATCAACATCAATATCTTTTTGACCAAA
GCCCAAAGGATAAATAATGCGAGTAATTCGATTATCTTTAACAAAGATTCCACCGACATACTCTGTGCTG
CGTTTAAAGTCTACACGCCGACGAAATGAAAAATATTCAGGCTCTTTATGAGCTCGGCTCATAGGACACA
ACGAATAACTAGAATAAGAGATGTCAAATCCTACGCCTTCAATATGAACTAAATTGTCATGATTAAACCA
ATTATAATCATATGCCAAGTCCATTAGATTGTCATATGTGAAAAGCACCGGATTAACATCATTGGTCACA
AGCATATAATTAGCAACTGCTATAATTTCATTATTTTTAACGAATACAAACCGTGATTGATGCGAATGGC
CTGGACCTGCGTTTTCACGATTAATGATATAACACTGGGCACCTTTATATTTGTACGTGTCTTTACACTT
GTGCATTTGATAAAGCATTATTCACCTACCACTTCAGCGATGATATTTTTGTTATTAAAGTTTTTATCGC
AATACAGAACATAATTATACTGCATTACACCACCAGACTTAAGCTGTTTTTGCACTTCAGCTTTCATTTC
AGGACGATCACGCTTAACGATATTCATAATATCTGCTTCAATTTGAGTTTCAACCTCAGTCTGATCCGCA
GTCATAGACCATTCGCACAAATCTTTATCATAACCTGCCATAGCAGGCTGAGCAGCACAAGAAGCTAAAG
CAAAAATTGTAGCAAAGATGAATTTTTTCATGATAATCTCCTCAGTAGTTTATGTTTATATAGTATCTCA
ATTTCCAACAAAAGTAAACAGTTATTTTAAAATTTCTGCGTAATCACATGTTACAAACTGTTTCTCTAAC
TTGACGATTTTACGAAAGTATCTTTTGCATTGGCGAATCTGCCTCTTCGTAGGGCGAACAGCAAACTTAA
TAAATTCCACTCGACCAAATGGAGGGCTTTCTTCTGCTGGAATATCTAACACCAATTCCCACGTATCTGC
AATAAGTGCTTTGAATTGCGTATTTTTCCTGACGTTATACGGAGTAGGTTTAAATAAAACAATATGCATA
TTATCCTCGGCAATCCACTTCACATACTTTCTTGTCATCAATGAAAGCTTTAACTAATGCTTTATTAACT
TCAGCATATTGAGTAGTAGCCCATTGAACGTCATCTTTCATCATTGTGATTTCTTTAGTAAACATGCTTT
CATTCTTAAACCACCCCATAAAAACTACCTTTACCAATTCCATAACAATCTCCTCATTTAACCGACAAGA
CTACTATACCATAGTCTTGTCAGCTTGTAAACTAAAATTTTAATTCATTCGCCAAAGCATCTAACTGAGC
TCGAGTCGATTCATTTCTTTGATAGCGATTCTGCTCAGCCTGAATCTGTTGTGAACCTGCTACTTCGTTC
ACTTCAGTTGGAGTAGAATCTTGTTCAATTTCTACCCATTTTTGATTTCCTTTTTGAACACCCATCAAAA
ACTTATTCCACTTATTCTTATCACCATATCGTGATTTGATTTGCTTAATGAGTTGTTGTTCAGCAGCTGC
TAGCTCCTCGGTTTCAATGACCGCAAGCATAAAATCGGCTGTTGCTGGAAGACCGGCAGATTCTGCAATA
TCGCTCATGTTAACATCGGAAGAGTCCCAAGCTTGTTTACCAACCTGTGCTGCAGTCCAAAGAACAGTTT
CGGTTTCAACAGCCAGAGCACGTAATTCCTCTGCAATAGCTTTAACAGTTGTGTAACTATTTTCTGAATA
AACTCTAATGCGGCAAGATTTACAAATACCTAGATAGTCGACAATAATGATTGTTGGAACAAAATTCTTC
TTGAGCTTCAATTCGTTTAAAAGTGATCGAAATGTATTAGCGTCTGCTCCACCAGTAGGATACTGTTTAA
CGATTAAACGACCAAGAGTAGATTTCTCACGCCATTTTTCCATTTTTCCTTTATACTCAGCGTAAGAAAT
ATGCCCATCATCAATGTCATCAAGAGAAACATCAAGCATATTAGCGTCAATACGTTTAGCACAGACTTCT
TCTGCCATTTCCATGGAAATGTAAAGAACATTATGTCCGAGCTGTAAATAATCTGCCGCTAATGAACACA
ATCCTAATGATTTACCAACGTTAACGCCAGCCATTAAAACGTTCAGTGTTCCAGTTTCAGCTCCGCCTTT
CGTAATTTTGTTCAGAATTCTGAGTTTAAATGGAACCTTACGAGCTTTATTCATATAAGATAGCCAACGT
GCTTCGTAGTCATCCATCCAATCATGACCAACGTAACTATCAAATGAAATTGATAATGCCTGGCGCATGA
TGTCAGGAATAGCACCAACATCCGGCATTTTCTTATTTCGTTTTTCCGGAGGAAGCTCAGCATTAGTTTG
AATTTCGATTATTTTAGACGTAGCATTAAACATCGCCCTTTGCTGAACATATTTTTCTGTTTCTTTTACT
AACCAGCTGTGGTCTTCCGGAGAATCAGCCAGTTTTGAAATAAGTGTTTTTACACCAGAATATTCTGTTT
CAGTAAATGAACTATTTTCTAATGCAACATTTAACGCATTAATAGATGGAACGCTATGGTACTCATTAAC
ATGAGATTTAATTAATTTGAATGTATTTTTAGCTGGACCACTTTCAAAATATTCTGAATCCATATATGGC
CAAACTTTTGAAAAATAAGCTTGATCAAATATGAGATGAGAAAGAATAATTTCTACCACACTTACTCCTT
AAAAGAATTTAAATTTTTTCTTTGACCTTTTATTAAATGCATCTTGCAGTTGCATTGTAATACATTTTTC
TACATGAGGAGCTAACTCAGCTTTTCTTTCTTGGTCAAGAACAGCAAAGTCCATTACAACCTTTCCATCA
ACCCAATCCAGTTTAGTTACATACACTATATGCGTAGAACCATCTTCTAGTTTAATGACAATCTCCTGGA
TAACATTTTCCATAGCAGATTTAATTATCTTAAGAGACTCATTAAAAAGACGTTCTTTTCTTTCTTCTTC
CCCCTCCGAAGAGGGGGATTCATCGATAATTTCTAGATCTAAATCTAAATCATCTTTATTCATTAAATTC
TTCCATATCACTTAGCTGTTCGAGGTCAGTTTCTAAATCAGCAGCTGATTTACTTTTACTTTCTGGAGAT
TTAAATTTTTCAACCTTTGAGTTAATCAATTCATCAACTTCAGCTTCAACAATTTCATTACTATCAATAG
CACCTAACTGATAAGCACGTTTAATAGCATCTCGGAATGGTTGATGCTTAAATAAAGGACCCCAGAATGT
AGTGCAGTTGGTATCTTTTGCACGCCAAGATTTTTCTTCGCGAATCATCTCGCCAGTTTCTTCGTCAAGA
AATTCACGAGCATACCAGCCATTTTTAGGTTTTACCACGAATCCTAATTCTAGAGCCATATCTAACAATC
CAGAATAAGGATCGATACCACCGTCAAATTTAACATCAATAAAGAATTTACTTTTTTCTTTAACGGTACG
AGATTTTTCTACATTTAGAACAAATTGATACCCCTGAAGATCAGAACCATCTTTAATCTGGCGTTTACCG
ATAATGAATACGGTATCAGCCGAATACATCGGTCCAGTACCACCTCCCATAACTGTTTTACTAAACATTT
CTTGTGTTTCGTATGTATGGTTAATAGCAATACATGGAATATTTTTAGTACTAAAATAGGGAGTTACGAT
ACGAAATAAGCTTTTCATTGTTTTAGCTCTAGTCATATCACTAACAACTTTTTCATTTAAAGCATCTTCA
GTTTCTTTCTTAGAAGCTAAGTTACCAAGTGAATCGATAAAAACGACTACCTTTTCGCCGCGTTCAATTG
CATCCAATTGATTAACCATGTCAATACGTAATTGCTCAAGTGATTGAACCGGAGTATGAATTACTCGTTC
TGGATCGACTCCCATAGACCGCAAATAAGCAGGAGTAATACCAAATTCACTATCATAAAACAAACATACT
GCATCAGGATATTGACGCATGTAAGATGACACCATTGTTAATCCAAAGTTTGATTTAAATGATTTTGATG
GACCTGCCAAAATTAACAGACCAGATTGCATACCACCAGTAATTTCACCAGAAAGTGCAATATTCATCAT
AGGAATTTTTGTTCGAACTACATCTTTTTCATTAAAGAATTTAGATGCTGTTAATTCTGCAGTCAATTTA
GAAGTAGAAGCTTTAATCAAACGAGATTTTAAATCAGACATATAATACCTTATAGTAGTGTTCTTGTTCC
ACGAAATACTTCGAGCATTCTTGCATAAGATTTATTTGGAAATCCAGCTTCAATTGCTAATTTTTTAAGC
TTAATAGCACCGGATTTTCCAGAGTTTTCCCATATTTCTTTAATCTTTTCAATGTTATCCCACATTATTG
GGTCACGTTTATGTGATGGTTTATTTCTATTATAACCATATGGATTATTAAGCAAAGCTTCTTTTCGTTT
TGCTTTAGTTTCTTCTGACTGCTTTTTGCCTAGCTGGGCTTTCCGGCAAACATCACTAAATCCAAGATGT
TTTGGTTTTCCTTTTTGAGCTTTTGAAATTTTTTCTTTGGTTTCTGATGAAACTCTATAACCAATTCTTC
TACCACCCTCTCCACCAATTTTTAAATTATAAGTCATAGGATCATTTACCACATCTATTGTTACTAATTC
TCTTTCAGCATCACGGGCTGATTTAAAATCTTTATAAAACCCAAGAATGCTTAAATTAAAATTTTTCTTA
CCATACTTTTTCTTGGCTTGTGCTAATAAAGTTCCTGATCCCATATAACCATCATTTAAATCATCGGTTG
AATGAGTTCCATAGTAAATTTTATTATTAACTAAATTAGTTATAACATAAGTATAATTATATTTCTTTTC
TTTATGTCTTTTCATGTCATTAATCAACTGCTATTCGATGAACTTCTCTCTTTTCTAATCAATATAACAA
AAGAAGTCCCAAAAAGCAAACAGACCTAAACCGATAATAAGCAATAAAGGTCCTAACATTTATTCCACCG
GTTAAAGATAAATAACTTTCTAATAATAGTTCATAATTTTTATAAATCAATAGCTTTTTTGAACGCATCT
TGCCATTCGGCTTTCTTTGCACGGGTTTTATTTAAAATATCATGTTGAATAGAAAGCATCTCTTTACGCA
AAACATCACTGTGTTTTAACTCATTGACTCTATCAATGAGTTCAGCACGATTATTTACATAAAAACGAGC
ATCATTAATAATTCGATGTTTGGTATCAAATTCTTCGTCAATTAGCATCACTGCATCAGATGCCATTGTT
TCCCAGACGCGTAAGGTAATAAAGTTGTCATTATAATTCTTGTCACCAATAATTAATGCAGCAATAGCTT
GACTATTCTTTTCAGATACCATGTTCATAGGAATTTTTCCAGTGAACACCGGAGCTTTGGTCCAAGGATA
TTTAGGATTTTTAAACTGTTTTTCTCGTGCATTGCCAAAAAACTCAATATTTAAACCGGTGTCAAATAAG
AATTCTACCATCTTGGATTCGCGTTGACCGGACCGAAATGAACCGCCATAAATAACATCCAAAGTTTTCT
TGGTAGGCTTAGATAATTGAAAATCGTTCATATGAATTTTATATTGTTCAATAGGAAAATATTCAAATTC
AATAACATTATCAACTTTCTTATGCGCAGCCTTAGCAATGTCTAAATTTATACCTTGGGAAATCACTTTA
ATTGGTGATTTAATTAATAGCTCTTCTTCAGTGTACAAATATGCCCATGGTCTATTTTTAACATTTGGCC
AAGACTGCGAAAACGGCAAACGTATATCTGTAAATAAATAATAAATTTTACTTTTGTATTTTGCCATAAA
TTTTTGCGCAGATAAAATTGCTAAATTAGGTTTACCGCCAAAAAAGTTAATAGAAGAATTAACAACTATC
AAACGGTCATAATCATTAACATCTACTTCATCAAAAGATTTAGTGTAAACACCATTTTTAAGAGAAATAA
TGTCGACATTAAGACCCATTTCAGAAATAACTTTAAAAAGATAAATAGTTTCAGAAGATGGAACAGTTTT
AAAATTAATAACATTATTACCCATATTAATTATAGCAATTTTCATATTATTCCTTTTATGTTAAACGATT
AAGCGTATTTTCCTACATAATCTTTTTTCGAAATATGTGTTTCGCCAGTTTTCCACCAATGATCAACCAA
ATAAAAATGACGAGAATACACATGTAGGCTTCCAACATTCCATATAATGGAACCTGCTTTATACTGGCGA
GTTGAATCACCTGCATTCAAATCAGATACTAATTTATCTAATACGTATTTTTGCCATGCATAATCATTAC
GGAATCCGAAGACCACGTCATTTGAGCGCATGTTAACAACCGCATTGATTTTCTTGTCACGAATCAGGTA
TTGTACTGTATTCGTGCACATGAAATCTGACATACCATCTTTATTATAGTCAAACTGCATAGATGGACGA
GTATAAATCATGATACCACGTCGAGAATCAGGATTTTGACCAAGTTCAGCTAAACACATGTCATACTGAG
CATAGTTATCTTCTGACCAGATAGCCCAACCATAATTCGAGTTAATTTCACCTTTAGAAGATGCTACTTG
TTGCCAAATCTTCGGTGTTTCACCCGGAATATCTTTAACGAACAAGCTTTTAGATTTATACCATTCAAGT
TCACGCTGAATGTATTCATCATTAAGAGCGCCAAAAATAAACGGTTCATCTGCTACAAATGATGCGCCAA
TAATTTCAATAGTTTTAACACCTGTTTTATCAACTACGAAATCTTTTTCTTTTAATGCAAGCCCCAAATG
AAGACGGATTTCTTCAACTGTCATAGAGTCACTAATCATTTAAACCTCAATTGATACATTCATATTTAAC
TTGTAACAGTAATAAACTCCAACCTAAAATAATAGTTGGAATCATAAGAGGAACCGTTACACTATAGTAT
ATACTTATTATAATCATCAAGATTAAAAGCAATGCTGCTATAATTTTGCTTTTCATTCCTTCTCTCTGAT
GATAATTACCTGATTTGGCTGCGCAGACTTTTTAGTTTCACCTGCAATTGACCAAATAAATGTAATAAAC
CAACCAATAATTGACCAGTTAAACAGTAAAGATGCGAAAAAGATTCCTACTGTCGATTTTGACCCACGCA
TCAAGGCGATAAACCATGGAAGCATGTATATAATAATAGCCAACACGCCTGAAACTAAAACCATAAAAAT
TGAACCTGCTACTAAAGTTTCCATGTTTTCCTCACTTAGGTCAAATTTTTTACACATGAATTATAAGAAT
TCACTACATACTCCATCGGAGCGTTTTTACCTGTACGCCACTGGTAATTATTAGCCCAATTTGCCCAAAG
CTCAGCGCAGTAGTTTTCAATTTTTTCTTCGCGTGTAATTACATCTGAATTACGATATGCTTGAGCAGAT
TCATCTGGACGAATAGCTTCGTCAAAATTCGCCTGCATTTGTTCTACTGTCTGTTTTGGAGCTTCTTTAT
AACACTTGACATTAGGATTATAAAATTTGCTTGAACAGTTTACAATTTTTCCTACATCAGACTGATTTAC
TACCGGTCCTTGAGCTACACAACCAGCAAGACCTAATGCAATAACCAAAATAGCGATTTTCATTTCATTC
TCCAAATCCGTATCAGTAGTTGATAGTTGTATAGTACCATGGAAGAACAGTCTTGTAAACAGTTTTGTGA
AAAAATTTTTAGGGAATCCAAAGGGTCCAGAATCATCTCTTTTTCATAAGTATAGATTTATATTACTTGT
ATGAAAAAGGGACCTGGAGGTCCTAGATTTATTCTATCAGCCAAACAGGAAGTCTAACGAAGCTTTTTCT
TCATAGTCCATGCCAGCCGATTCACACATACCCGCAAGCGGTTTAACAAACGATTTTTGGAACAAAGTTG
AGTGGTCAATCCAAGATAGCACATCAGAACGAATTTCTTTTGGAAGTTCTGTACCCGATGGCCAAGCAAT
GCACTTGTCACCAAATGGATTTCCTTCACGTAATGGAAGAACCATTACTTTATTTCCATCCAAAATTGGA
GCTACACCTAAACCGCTAACAGCTCGACGATAAGTTAGCACACCACGAATATGGAACGGGCATTTAAATC
CTGGCCAACCTTTATCATCATATTTCGCTATATCGTTCGCAGTTTTTACTTCAGCAATAACTTTATAGTC
AAGTTGACGATATTCTTTCTCGAAGTTCTTGTAGTATTCTTGGACAGACTCTTCACCTTCCTGAAGAATA
CGACGAATACTTTCTTCGAGAGCTTCTTGCACTGCTTTTGGTGTTGAACTCTGCTGAGTTTCCATACCCA
TGATTTTTAGATGCGGTTCAGCAAATCGCTTATCTTCCATATCATAAACGTTCAGAGCATAACGCTTTTT
CGCTTTCCAAAATCCACCAACGCCCTTTGAACCAAGCGGAGGGCAAGAAATAGCTTCACGGTCCATATGC
ATCAGATGCTCGCGGTTATTCATATAATCACATAACTCACGATATGCAACATCAATCATAGGTTCCATCT
TTTTCTTACCGAACTGATTCATGAATTCAACCAAATCGTTCTGCTCTTTGAATCGGTCAAGACCAACTTT
TTCAATAACTTTATCTACGCAAACATATACCGAATCAGTATCACCTGCTGCAATGAAATCTTCATCATTA
GTTCCGCATACTTTATTCAGATATTCATTAATTTTACGAGCAATCCACTGAATACCGACTTGGCCGAAAA
TTGTGATAGCAGTAGCATTTCGCAAATCATAGTAACGGAAATGAATATTACCAAGAGCACCATAAAGACT
GTTAATGAGAATTTTACGGTTCAGCTGATTTGTATTAGCAAGTGTAGCTGCTTTTTCACATTCTTCAATC
AGACTATTGAGAACAGATTCGGTGTAATTCGATAGTTCATTTAAGAAATCATCACTGAACTTAACATATC
GTTCAACTTCTGGTTTAGTTGAACAAGACCCTGCGCCTTTCATAATAATCTTTTTAATAGCTTCGGCATT
CATTTCTTCAGCGAACATTTTCTTTTTCCAGTCTTTACGCTGGAAAAATACTTTAGCGATTTCCTTTGGA
ATGATACCTTCTTGATGTTTATCATACATCCATCCATTCGGAGAACAAGAATATTCATCACTCGGTTTAG
GAGCTGTTCCTGCGATATATTCATGAATTGGATGAACTTTAAACTGACCACGAATAGTTTCAGGACTAAT
GTTAACCTGGCGAATAATGCTCGGATACAGAGACGTCAAGTCAAAACTCATAATGTATCGACGTGCAATT
GGTTTAGGTTCAAACACAAATGCACCCGGAAAACTCTGTTTAACGTGCGAACCTTGTTGAGGAATAACCT
TATGTTCACCTTTCAATGAGTTAAAAATAATAGCATCCCAAGTTTTAATAGGACTCATTACACCAGAAAA
AGGCATTTTAGCGTAATAAGACATACTTAAAACTAGATCGATAAACCCACGAATTTTATCGATTGCTTGA
ACTGATTCTACGTCAATGATGTTATAACTAATGTATCGTTGATGATTAGTCTCACGAAGTTTATTAATAG
GACCGTCGTATGGTAATTTACCTTTTTTGGTTTCATGTTGAGCAACTGATTCCAAAGAGAATGACGGCAA
ATTAGTAAAAGCGAATTTCTTGTACAAATCTAAATAATCAAGAATAGATACGCCATCAATAGAATAAATT
TCTTTGCTACCGTACATATTTTGAATTAGTTTAGATTTTACCCGACCGATTGGAGAGAAACGTTTCATAC
TACGTTCACCCAGAATCATTTTAACACGATTCATGATATACGGAACGTCAAACCCCTCAATATTCCAACC
AGTAAAAATAGCAGGTCGTTTCTGTTCCCAAAGATTGATATATTCCATGAGCATATCACGCTCATTATCG
AATGGCATATAAATTACTCGGTCAAGAATTTCTTGAGGAACTTCATCACCACCTTCACAGTCAAGCTTAG
CAGCTAACTTTGCATCCCATTTTGATACTGAACCGTACATTGAATTCAAAAGGTCGAAAACATAAAAACG
ATCGTCAATTGAATCGTAATGAGTGATAGCATCAATTTCATATTCTGCTTTCATTGGGTCAGGAAATTTA
TCACCAGTAACCTCAATGTCACAGTTAGCTACACGAACAAATTTTCGGTCATAAACAATTTCTGAACCAT
ATGTATCACTTATATAAGCGAGTTTAAAATCGTTCATACCGAGAGCTTCGAGACCGATGTCTTCCATTCG
CTTCATCCAATCTCGAGCATCTTTCATTGATGGAAATTTTTGAGGAGCGCAGTTTTTACCATAGATGTCT
TTGTATTTTGACTCTTCCTTACAATGCCTAAACATAGTTGGAAGATATTCTACTTCACGGGTACGTTCCT
TTCCATTTTCATCAATATAACGTTCAACAATGTTATTTCCGACTGTTTCAATAGAGATATAAAATTCTTT
CATAGATATTCCTTAGTTTATAGCCCGAGTTATTAGGCTCTTGATATATTATACTCCAAATAAGGGGCCG
AAGCCCCTTGCTTAATTACCAATCGTATATTTAGGAACGAGCTTCCATTCATGTTTTTGTTTAAAAGAAA
TAACTCGGAAGTTATTAGTTAAATCTTTCATAAAAGTTCTTTGACCAGGAACGATTTCAATTAGTCCCCA
ATCTTCTAATAGCCATGCAATCGAATCACGACGAACTTCATCTTCTTCTGTCATTTCAACTTGACGACCA
TCCATACGAAGCATTTCTTTAAAATGAACGATATAGTATAGTCCTTTTTTCTGAAGAATATGACAGGACT
GATATAGAACTTTATCTTTATTATTAGCAATTCCCATACGAGTCAAAGTTTCTTTTACTTTCAGAAAATC
TTCAGGTTTTTTAAGAGTAATTTCAATCATTTTACCATTCCAATGCTAGTTTTTTGAGTTGTTTCTGTTC
TTTTACGTTCTTAGTCACTTCTTTCAAAAAATCATCCGTGACTAAACCTTTTAGTTCTTTTAATACTAAA
GGAAGTTTTCCATTTTTAGTAAGAATTGATTTATAGTTAATTGCATCATTTGTATTAACTTGATACCGCT
TAGCAAGTAACTTAATAATCAATACTTCGGTGGAATCTTCAACCAGTTTTGCCCATTTACCATATCTTTT
ACCACGAGGAACTGCAGCCATTAGATAATTAAAATGAGCTTCATCACTTAAGCCTGATCCAATTAAATTC
ATAGCATATACAGCTGGCATACACTCTGGAAATTGTGATAATGCATTTTCAACCATGAATTTTGAATAAT
CTTTTTGAGCAATAGAGCATTTAGTTTTATTATTAATAGCTCCAATTATTTCAAAAAATTCATTTTCTGC
TTTTTCTTTAAAAGAATCAGCAGCGGATTGGACAGCTGTCCAATCTTTTGAATACCAAGCAACTTGATGC
TCGTTTAATTGAATATCATCTTTAAATAAGCTCATATCACTTCCACTGCATTTCGCATGCTAATTGAATG
AAAAGATAAGCTAAATGCAATTCAGTATTAGCTGCAATACCATGATACTGATTATTTTCGCCGACAATTT
CGTACATACGAATAATACTTTGTGGAGTTACACGTGAATAGATTTCTTCGGCAAGTTTACCCACGAACCA
CGAATAATCAGCCGCATATTTTGGTGCTAAAGCTCTGAGTTGTTTAACATCTTTATTTTTGAGAGACTCA
AGAACATCATCAATAGCACCACGATCGTTAGTAACCAGTGATAAAATACCAGCATCCAAAACACCTTTAG
ACGAATAACTATCGAGCTCGCCAATAGTTTTACGAAAATCAGGAAAATTCTTTTTAACCAAAGCTGCTAC
AACTTTCATATCAGCTATAGCAATTCCTTCATGCTTGCAGATTTCAGTCAATCGACGAATCATCTGCTTC
ATCATTTCAATTTTATCTTCATCAGTTGGTTGACCGAATGTAATAACTCGGCAGCGTGACTGAAGCGGTT
TAATAATACCATCAATATTATTAGCAGTAATAATAATACTACAGTTTGAACTATAAGCTTCCATAAAGGA
ACGAAGATGTCGCTGAGACTCTGCTAACCCTGAACGGTCAAATTCATCAATAACGATTACTTTTTGACGA
CCATCAAATGAAGCGGCGCTGGCAAAATTAGTCAAAGGACCACGAACGAAATCAATTTTACAATCTGACC
CATTCACAAACATCATATCAGCATTTACATCATGACATAATGCTTTTGCTACAGTTGTTTTACCTGTTCC
TGGAGAAGGAGAATGAAGAATAATATGTGGAATCTTACCTTTACTTGTAATAGATTTAAAGGTTTCTTTA
TCAAAAGCGGGAAGAATACATTCATCGATAGTAGATGGACGATATTTCTGTTCAAGAATGTGTTCTTTTT
CATTTACAGTAATCATAATTTCCTCATTCAAGTTTTAGTGTAAATTATAAAGGCCGAAGCCCTCTATTAA
AAATCGTGGGTAGAATCAGCTTCAAGAGCTACCACATAATTCGCGTGTTCACCTTCAAATTTAGCAGCAC
CTTGTTTACCTTTTGCCCAAAGCAGAAGTTTATAATTTCCTGGTTGCATTTTCATATTTGCCATATTGAT
AATGAAATTAAATGTATTTTCACCATCATAATCACCAAGAGTCAAAGAATATTTAACACGGGTCAGAGCA
GAATCTTCTACTTTATTAAAACCGTTAATTACGATTTTACCTTCTTTTACCGTGATAGCAATTGTATCAA
TTTGCAGACCACGAGATACACGCAACAGCTGTTGAAGGTCTTCAGCTTTAATTTCAGTAACAGCAGATGC
TACCGGGAATGGAATTGGTTTATTAGGAGCAACTACTGTACTCGGATCGGCTGCTGGCCAAAAAATTGTT
GAGCGGGCATCAGCAATTTTAATATTTCCATCTTCTGACTGGGAAATTTCTGCATCATCATTAACTAAAG
ACAGAATACCGAGAAAACCGTTCAAATCGTAAATTGCTACATCAAAATCAATAACGTCAGAAATATTTGC
TTCCGCATAAGTTGTACCATTAACTGCGCGAGTCATAATAAATTGACCGGATTTAAGCATAATACCAGAG
TTAATAGTAGCGAAATTTTTAAGCAGAGCAGTAGTATCTTTAGACAGTTTCATGTAATTTCCTTCAATTC
AAATGAGATTTAATTTTATAACTAATTTAATAAAGCAATTAACGATTAAAATCAGCCGCAATTGTTTCCG
CAACAATTTGAGCAGCAACAATTAGACGTTCATCTGCATTACCGCAATAATCATCTTCAAGGCGTTCACC
ACATGAAGTCATAATAAATTTAGCACCGGCGTTTAGGGATTCTGTAGTATGTTTGCGCATTAGTTCAATC
CATTTATTACTTACTTCACGATCGATAGCTTCATAATACGCATGACGAGCAGGTGCAGATTTAATTTTGT
TCTGAATAACTTCCATTGCGTTATCAGAAAGAGACAAAACCCATGCTCGACGAATTTTATTTTGGTTTTG
TGGATTTGATTCAGAACGCACGTGTTTTGGCTGAATATCTTTTACATCAACAGTATAATTCACAGTAATT
TTAGTCATAATACACCTTTAGTCATAATAATCAGTAACAGTCCAAGCTTCATTTCTATTGGACATTATTT
TTGTATATTCTGCTTTAAATGCATTCCTAAGCATAGATTCAGTAACTATATGCTCTTCATTAGAAAAATT
ATTTCTCAGAATATATCGTTTTATTTCAGGAATAGTTAATAGATGCTGTCCAGTTGAATATTCCATGTTT
TTCCTCCATAGAGATTATACTCTAATAAATTAAAGCATAATCTCTTATAAATTAAACCATTACAGTAAAT
CGACCAACTTTCTTCATTTGAAGATGCTGACCATATTCTTGCGGGTCATGGTCTTTATGCGAAATTATAA
AAACGTTAGTGTTTTTCATTGAATTTATAATATTAGCTACACCTTTAATACCTTCGGCATCAAATGACCC
ATCAAACACTTCATCAAGAATTAATGTACTAATACTAACACCAGATACGATAGAAGCAATATCACGCCAA
GTAAATAAAAGAGCAATATCGATTCGTGCCTTTTCACCTTCACTAAATGAAGCATAACTAAAATCTTCAC
GACCACGGGATTTAATTGTCTCATTAAATTCTTCATCTAATGTAAACACATAATCCGCTTCCATTATTTT
AAGATAATGGTTAATCTGCTTATTAAATAATGGAATGTACTTTTTAATAATAGCACCTTTAATACCAGAA
TCTTTGAGCATATCAGTCAAAATTCCTCGGTGGTATTTTTCCATTACTAAATTAGTTTTTGTCTTAACAA
TTTTATCAAGTTCTTCTTGAAGCAGTGCTATTTCATCAGCATGGTCAATAAACTCAGAAGATGCTTTTTC
TATAGCCGCTTTAACTTTTTTAGCTTTATCTACTGCTGCGATCAGAGATTGCTTTTTATTGCGAATATCA
TTTGCCAACGACTGCTGGGTTTTAATATTATCTCGGTATTCATCAACAAGAACTTTTAAATTATCACGAT
GTGTTGAAAGCTGTTCAAACGAATGTGTGCATTCAGAAACTTTATCTTTAATTTTAGAAACAACTTTATC
ACCGGAACTCAATTGTGACAAACAGGTTGGACATAATCCACCTTCGTGATACATATTAATGACTTTATTA
TACGAGTCAATTTTTGATTTAATTAAAAATGCTTCTTGACCGATTTTATTAAATGCATCAGTCGGGTCTT
CGTCCAAAACAATATTAACTAATCTTTCGTTAGCTTCTTCTATTTCCGATTTTAGCGTTCTAGCTTCTTT
TGCCAAATCATCATACATATTTTGTAGACGAGTAAGGTTGTCACCCGTTAATTTTTTCTGGCGTTCAACA
TTATCATTATATATTTTAATTTGTTGGATAATACTATCTTTTTTAACATCAAGCACTTGGTTCTGCGAAT
TTAATTCACGTATTAGTGCTTTATTAAGCTTATCCATTTCAGCTAATGTTCCTACCTCAAGCAGGTCTTC
CACAAGCTTTCTTCGCGCAGGGGTCGACAAACCCATGAAAGGGGTATACCCTGCTGTACCAAGGACAACA
ATCTGCTTGAAACTGGCATATGACATTCCGATAAGCTGTTCAAATTCTGCTTGGAAATCTTTACTGCTGG
CAGATTCATTAAGACGTGTACCGTTAACGGTGATTTCGAAAACGTTTGGTTTTTGTCCTCTTTTGATATA
GTACTTTTTCTCATCATATTCCATCCACAGTTCAACTAAAAGTTCTTTCTTATTTGTGCTGTTTATTAAT
TGACCTTTCTTTACATCGCGAAATGGCTTACCAAAAAGCCCAAATGTGATGGCTTCTAGCATAGTAGACT
TACCACCGCCATTTCGTCCAGTAATAAGAGTTTTTTGAACCTTATCTAATTGAATGTCAATCCCATTTTG
ACCAACTGACATTATATTTTTATATTTTACTCTATTAAGTTTAAAATTCTTCACAAAAGATTCCTTTTAA
TGTATCTTTTAGACCATTCTATCATATCATCATAATCTAAAAAGTATTCATCAAATTCAGCCATGCAAAC
AACGCCTTGTGCTGTTTTTGATGTGATATAAATTATTCCAACATATCTAGAATCTTCTTCGGTATAATCA
ATATTTGCTATGAATTCATCATTAATGTCAAATGTCGAAAACTTCACAGTATGCATCCTTAATACAAGAT
ACAGCCATATCTCGTAATGATTTAGGTGTGTCATTATCTAAAATGTTGAAGTTAAAAGATACAGCCCAGT
CAGTGCAGACAGTAACCTTTTTAATACAATTATCTTCAACCTCTAACGGTTCAAACCAGTATTCAATAAT
TTCTTTATGACCAATATCATCTTTTACTTCACATTTAAAATGCTGACTCATCATAACATTTTTAAATTCA
TCAAAAGTCATTGTGTTGCCTCTACATATAGCTGATTTGCATATTGAATAAGTGCTTCACGGTCAGAATC
AGTGATGTCTGGAATTGCATTAATATACTCTTCCATTAATGTCTGAAGCGATTGAACTTCAACTTCTTCA
CTGTCATCTGACTCGACAGAGTTATCAATCTTTGACACAACTCGTAATGAATGCACAACTTTTTCTAGTT
CAGATTCGAACTTCGTCAGATTTTTGTCTACTTCAGTTACTATAACACGTACTGATAGATTTGTAAAATC
TTTATAGTCAATTTTTCCTTTAAATGGATAATGAATTCTACGATGCCAGGTAGTATTGTTTGGAATAAAT
TCCGTTCGTTCTGTTTCTGTATCAAACATCCAGAACCCACGAGGGTCATTCTCGTCACCTGCGGTTAGTG
TCCATGGTGTCCCAATATATCTGACGTTTGCAGCCTCAGAAATAGTATGGAAGTGACCAGACCACACTTC
TTTATAAGTCTTAAGGAAATCGGGTTCAAGACCATGAGATTTCATTCCTTTATAAAAATAAAATCCATTC
AGTTCCCAGTGACCAACACAAAAAGAAGCAGATGAAGTTTTGATATGCTCAAGAATTTCACCAGTATTTT
CTTCGCACATCCAAGGAATCAAATCAATCAAACACCCGTCAAAATCTACTGTAGTAGGCTTATCATACAC
TTTAACATTAGGATATTTAGCCAAAAGCTCAGTAGAAGCATTTGGATGCATTACATTTTTATAGTGGAGA
TCGTGATTTCCTACAATAGTGTGTAATGTAATTCCAGCATCATCAAGCGTTTGAACTATTTCACGGGCAA
ACTCCATAGTTTTATGTGTGATCGCTTTTCGCACATCAAAAATATCACCGTATTGAATCCAGGTAGTAAT
TCCATTTTTCTTAGAATATTCTATCGCTTGCTTAATTCCATCAATTTGAATACCGCGAATCCACTCATCA
TCAGCTTTAACGCCTAAATGCCAATCACCTAAATTTAAAATTTTCATATATCAAGAACCGTCATTGAAAT
GCAAAATAAAATTATTGAAATAAATCCATCTGGAGTGCTAAAGAACCCAATCCAACATGCTCTAGTGAAT
AGATAAAATGCAAGAAAAAGTATCACATATCCAAGAAATATCATTATATCAAACTCCGTATAAAGCTAAA
GGGCCGAAGCCCTTTATTTTGTAATAATGTCAAACTGTTCTTTAAAGCAGAAGCTTGAATCTTGATGCTG
ATACAAAAATTCATATGCTTTTTCTCGCTCACGGTCATAAAGAGCTCGGTCAGATGACAGTTCTTTAATA
CGTTCAAATGTTGATTCCATATCATTTTCATCAAACCAAATGATACCGCTATCATGCGAGGTCAAAGGAG
TATTATCAACACGGAATTTTAAATTTTCGCCAGTAGATTTCCAAAATACCGGAATTGTTCCACATGCACC
AAGCTCGAGATGAGTATATTCGAGTGAGCGTTGTAAGTATTTCTGGTTAAGTTTACTCAACTGATATCCA
AAGCCAGATTTACTCATTCGTTCAAGCATTTCACTATTAATATAACAATCTAGGATTTGTGCCGGTTGAT
TCGGCGCGAGATTCATTTTATCAATCTCACGATTACCGTAATATTCATACGGAATACCTTTTTCCTTAAT
TGCAATAAAAGCAGGGGAACGTTCCAGACCTTCCATTACAGTGGATTTACCAGCAGGTTTTAAGAATTTT
TCATGAAAATCAAACATCTGGTAAAAACCTTTCCATGTAGTCGTACGACCAATCCAACGGTTGATATTCA
TGTTAATTTCAGAAACATCTTTCCAATAAGTTGACCGAACCTTCACAATATCCATAGGAGGCTGAAAATT
ATATACTGTCGGTGCTTCTTCAATATCATCAAACAGAGAAACAGTTTCTGGATACCATTCTTTCATCAGA
ACTTTATTAAAATCACCATTATCAGAATGGCTAAAAATAACATCAGCTCGACGAACAGTTTCTTCTAATC
CCAAATTTCGACGCAAAGAAAGAACAGAATGATCATGCTGATAAACTACAACACGAATAGAAGGTTTAAT
ATTATCTAAAAGTTTTTTATAGTTATTAATCGTAGCTTCTTGAACGGAAGTAGCAGGAACAGAATTAATA
ATTAGAATATCACAATCATTTACTAGCTTAAGTGCTTTATCGTATTCTTTAGCTAAAATAACTGGAATTG
AAAATGATTTGTGGTCATGAGAACTTGTACGAGTAAATGATTTATCTTTAGCATAAACCAAAGTTACTTC
ATGACCATTTTTAATAAACCAATCACGTTGCTCGAGTGAGAATTTTGTTACACCACAACCTTCAAGACCT
CGAGCCATAAAAATGCAAATACGCATAGTTTTCCTCTTTTCATTTAATAAATCATGTAAATAATATTTTA
TTTTCTATAAAACGCTACGAATAGGCCCAAACATATCCATAAGCAATTTTGCTTTTTCCAGATATGCACT
TTTTAATATTAGACATGCATCCTTTAACGGATACAGCTGCGTCTTTAATTCTAGGAAATACTCGTAATAA
TTTTCCAGTAGTATCATATTGAAATACTGGCACATTTCTTTTCTGAGTTATCCCACGTTTAGATTTAGAC
ATTTTTTGTTTAGTTGCATTAGACATCCTAGAGGGCTTTCCTATGTTATCAGAATAATAATATTTCCATT
GAAATCCTCCAGCGGTTTTCCTTTTACCATCTACACATTGTTTAATTGAAGTTGAACAGCTATATGACAT
ATCTTCTGCAGCATCTGTAATACATCTATATTTGCGAATAAAATTTCCATTTAAATCATATTGATAAATT
GGTTTTCCAGCATTTCGTCTAGCGTTAGACATGTATTTTTTATTATCATCATCTTTCCAGAACTCTTTCA
TGCTTTCCGATATTGAAGAAGATACAGCTGTTTTAATCCATCCATACATTTTATTATTTAGTCTTGTTCC
GTCAGAACTATAACACATCATACGAATAGCTAAAGCCAATTTAGGAAGTCTATAAATTTTAAATAATAAT
AAATGCGCGGTAAAATGTTCTTCTGGTGTCAAAAGAACTAAATTAGTTTTATCATCTGTACCACCCATAC
ATCTAGGAATTATATGATGTGTTTCAGTATAGTATGTCAAAAGACTTTTATCATTGCCTCTGTTTAGTCC
TTTTTCGATCAGTAAATTATATATGTTTAAATAATTCATTTTAGTTTATTTTACCAAAAAATTTATAAAG
CAATATAGGAGCCGAAGCTCCTATCCACATAATACGCCATACAGAGGCTCGTTAGAACTTTTAAATTTTA
TGCGCTTATATGTTATAGTTCCTTCTGCTTTAGCTTTATCATGAGCCTCTTTAAAGCGTCTCATCATTTC
CTCTCTAGAGGAACGAATTTTATTATAATCTATTTCAGAAGTCGGATGTTCATCTTTCACAGTTGCCACC
ATTTTTTGACTTGATAAGAATCAACCCACACTTTCATATTAGGGTCTGCTCTTACAATAGGAGGATTAAC
TTCTTTGATTGAACTATCACGGTATTCTTTTTCAGAAATTTTCACGCAAAGATGACCATTCAAAGATTCA
GTAAATCCTGCATTAATTTTAAGACGCTTGAATTTTACGTGTGTAAGCATCAATCATATCCTCAATCTGC
GATCTAGTAGTCTTCCAAAGAATACTGATGAGTTCATCGTTATATGGCTGTTTTAGAATATCCCGACTTT
TCTTGATAGCATATTCGTATTGAGCAAAATTATTATTTTCAGCTGCGATCTGAGCGTGCTTATAAAGACG
ATTCAGTTCGCGTTTGTTTTTAGACAATAACTTATTTGCTTTTCTTTCTGCTTCAAGACGGTTCTTTTCT
TCAATAGAAGAAATAAGCTTTTCCACTTCATCATTAATTTCGGGTTTATCAGTCATATTATTTCTCTAAT
ATAAAATAAAAATCATCATCTGTTAAATGATACCGATAGTTTAATTCTACACCATTAGATTTAAAAGCGG
TATCATACGGATTTTCTGGATCAATATCAATGTCAAGAGCTAAAACTTCCCTGAGATACATTTTAAGTAA
ATAGGGAATAGCTTCAACTTCAGGTATTTCTTCCAAGAATCCGGAGAGGTTAATCGTTAGCCTCATATAA
AAAATCCAAACTAGGAGAATCATCTACAACACTTTTCTTTTCAGCCCCCGGTGTTCTATAGGTTGATTCT
TCGTAATGCGTCATTTTATCATAGATGTCTTGAATAAAAGTTTCATCTACTAACGCAACCATATCGTCGT
CACGGCTGTCATAGACATTGTGAACGAAGTAACTATATTTCTTTGCAACTTCCTTACGTTCTTTTTTAAT
ACGTTGGACGAATGCATTAAAACAAGCTTGAGTTATATACGCATGTGGGTTTTTATATTTCGTTTCATCA
AAATTGTGAAGCCCCTTAATAGAAGCTTCTATACCATCTGCAATCATTTCTTGTTTCCAAGACTGGGTGT
ATCCTGAAAAGTTGAAACGTTTAGATAAGCCTTCTGCAATAAGCATAATGGCTAATCCGATAGTATCATT
CTGACGAACTACTTTATTTGGGTCTTTATTATTTGCTAATTCTGTTTTCCAATCAATAATAGCTTGTAAA
AGCTCTTTATTGTTTACGTAATTATATTTAGGCTTAGTTTCTGACATTTTCACCTCTTAGCTCAATTCAT
AGATCTATTATATCATAATATTTGAAGACCTATCTTAAAGCATAGAGGATGAATCAATTTCAAGCACTTC
ATCAGATTAGCCGCTCCAAGAGCTGCATCTAGTGAATCAAATTGGTCAACATATTCAATTAATTCGCCGT
AATTAGCGTATAACCACCATTGGCTAAATTCATACTCAAGGATGAATCCATTTCCTTCAATTTGAGTTAA
ACCAATGCCATTTGTATTTACTTCATACCCAGCGAGACGTAAATCGTTAATAAGAGCTTCGTTCATAATT
ATACCTTAGTAATTTTCAGGTCTGCAAATTTTTTCTTGCGTTGATTTTTCATGCGACGAATAGTTTTATC
GGAAATTTCATGCTTTTGATAAGCTTTAGATTCTACACCAAAAGCTTTAACATCAAATTCTGACAAGATA
TATTGAACCAACAATTCACGGACAGTATTGCGTCCAATCTTCTGATCATTCTGTTTCATCGTTTGATGAA
GCTCTTTTTCCCATTTATCCAAAATTTGAGGAGTTACAATATCGCCTTTTTCTAGCAAAGAAACTACTTT
ATCATATGCGTAAGAATTGATGGCGTTTTTAATTTGAATAGTCATACATTATCCTCAATTACGTTAAAAT
TTTATTATCCAAAAAGGCCGAAGCCCTTAGGCTAAACTTTTTGGCACCCTTCCAGCCTTCGTACATCATT
GCGACTGACAATGACAAAGCTCCTTCACATGCTGCATACTTATTATTCCAGAACCAATTTAGAAAAACTT
CATCCTCAATACCGTTGTGTTTCATGTTCTGGAAAAATTTGGCGCGTTCTTCAGCAATTCGCTGCGATAA
TTTAGATTCAGGATTCATTTAAATTTCCCAATTGCCATTTTCATCAATAAATTTAATCCAGTCATTTACT
GACCACTTGGTCGTATCGCCTTTTGGAGTAACATTTAAAGTGTATAGCCCTTGCTTAAAAAGCATGCGTT
TGATATTCATATTTTCCTCAGCTGTAACGATAACACTCGTTTGATTTACGTTTAGCAACTCGTTGAGAAG
TATTATAATCAAAATCATCATCAATGTAAACTGATTTTTTCAACTTTCTTACTTCACCGCGTAATTGACG
ATTTAACTCATCTTTAACTTCTGAATCAATACCTTTCATTCTACGCCATTTATCTGAGCGAAAAATGTTT
TCAACCATATCTTTATGACTTACCCCATCAGGAGCTTTACGCTTTCCGAAATAGTCATAATCACGCACTT
TTAAGTCTTTACGACGATACGTTTTACCCATGGAGTTTAATTTCCTTAGCAACTGAACTAAATACAGCAC
GATCACAAATCATACGTTTATGTAACTTGAGTATAAGATAGTAAACATCAAAACCATTTACATAAGTAAC
ACGAACAACATTACCATTCACGAGATAATACTGTCCCTTTTTAATCTCTTTATCAACCACAACCATATCA
ATTCCTCAAAGGTAATTCATATGTTAATAATACCACGGTTTGAACTTGTTGTAAACAACTTTGTGAAAAA
TATTTTAGGGAATGATAAGAAAGGAACGATAGCTTAGAATGGTAATATACAGAATGTGAGAAAGAAAGGC
CCAGAGGGCCCGTCTTAATCTTCTATGATATCTCTATCATATCCAAGTGAAATGAGAGTTTCTTTGAAGT
GTTTAATGTTCTTTTGTCTAGAATCATTAATGAAAATGACTGGATAACGAATGTTAAGAGATGTGAATCC
AGCGCGTTTAGCAAGAGATACAATCAGCGGACGATCATACTCAATCTTACCATTATTTGTAAGAACTTTA
TAGAAAGTAAAAGGAGCATTGAGCTCCTTTAGAAGTTTTGTAACTGATTGACATCCAGGACAACGACCTA
CTTCATCTGGAATTCCATAGACTTCAATCTTATTTTGTTTCACAACTATTCCTTACTAAGAGCAGCATTC
AGCTTGTTAGTAATTTTATCCAGACGCTCATTGAATTCACTAGAAGATAAGCCCTTTTCTGGAGAAATCA
AACTAATCACGAAAAATATAGCAATAAAAGGAATAAGGAAAATAGCTCCAACTGCCATAAACAGAAAGAA
TGTTACAGTTGTAAGAAAATCAGCTAAACCTTTACGAAATTTATACATTTACATTTACCTTTAATTGATT
AACCAAGCATTGATAAGCACTAAACTATACTGCGAATAAAATTCTGGACCAAAATGAAAATCATATCATT
TATAGTATCCATAATGTAATTCAATTTAATCATGTTTCCACACCCCATCGGTATTTGACCAAAGTCGCTG
ATTATCTGATCCTCGCCACAGCTTTTTGGTCGGAAGATTTTTCTCATACTTCCCATCAATAATAACATCA
ACATATTTAAGCATTTCTAGTTGTTTAATATCTTCAAACTTATATCCTGTCCACAACCAGATATCTTTCT
CCGGAAATCTTGCTTTAACCCAAGAAACTAAATTTGAAATCTCTTCTCGGTTCTGTGGATAAAGTGGGTC
ACCGCCGGTTAAGGTAAGGCCTTGGATATACGATTTGCTTAAATGGGACGCAAGTTCTTTAACGGTATTC
ATAGTGAATAACTGTCCGTTACGAGCATTCCAAGTACTACGATTATAACAACCTTCGCATTTATGCAAAC
ATCCAGTGACGAAGAGAACGACCCTACATCCAGGGCCGTTAACGAAATCGCATGGATAAATTCTATCATA
ATTCATCTTTATATTTCCAAGTATATCCCTTATGAATAGATTGAATTCCTTTGCAACATTTATAAACAGC
AGAATGAAAAAATCCTGCATTTCTTATTTCAGTTGCTCCAGTTAGTTCTATTGTATTTCCGTCTGGTGAA
TAGCCAATAACCGTTTTGGTGTTAAAGCGATTTCTAGAACTATTTTTGATATGCTCTTTATGATTAGTAG
ACAAAGTTCTTCCTGTTAGCCCATCAGATATCCTTTTTCCAAAATCATCAGGAAGAGTTACTTTAGACCT
AGCTATGCTCATTAGCTTCTTAGTTTTATCGCTTCTTTTTGCGCCACGCTGATTTTCAAATTTCTTAGCT
TTTGCATACGCGTATTGTGAAGAAGTAACTTTTCTATGCTTACTATTACTCATTATCCAATATGCATCAT
ACATCTTTCCACCATATATTTTAGCCAATAAATGGTGAGCAGTATAGTGGGCCTTATAAGTTAAAAATAC
TAAGTTGTCTAAATCATCTGAACCACCCATACTTCTTGGAATTATATGATGAAGTTCATACCCGGCTTTA
GTTTGACGGGGCTTAGATGGGTGTTCAGCAGAATTAACTAGATTTGAATAGATTCTGTCATAATTCATTG
GTGCTTAACCCTATGCATGATTTCTTTATTTTTACCGAGATTAAATCCGCGTTCGTTCGGATTTCCCAAA
TAACCACATGTTCTTCTTATGGTATTCATCTTTTTAGGATCAGTTTCTCCACAAATAGAACAAACAAATC
CGTTTTCAGTAGGAGTCATTTCATGGGTACTTCCACATGTAAAACATTTATCTACTGGCATATTAACACC
AAAATAATCTAAATGCTGTGCAGCATAATCCCACACGGCCTCAAGACCTTTTAAGTTATTTTTCATATCA
GGAAGTTCAACATAAGAAATGTGACCACCTGTCGCAATGAAATGATATGGCGCTTCACGAGAAATCTTTT
CAAACGGAGTAATATTTTCTTCTACTGAAACATGGAAACTGTTAGTGTACCAGCCTTTATCGGTAACATC
TTTTACGCTTCCATATTTTTCTGTATCGAGTTTACAGAAGCGATAACACAGGTTTTCAGCAGGAGTCGAA
TATAAACTAAAAGCAAATCCGGTTCTTTCAGTCCACTGTTTAAGATGAGCATTCATTTTAGTTAAAATTT
CTCGTCCAATATCACGACCGACAAGAATATTCAATTCGTGAATACCAATGTATCCTAAAGACACTGAACT
TCTACCGTTTTTAAATAACTCAATTATGTCGTCATCAGGTTTAAGACGAACCCCGAATGCACCTTCTTGG
TAAAGAATAGGAGCAACAGTAGCTTTAACTCCTTTTAAGGAACTAATTCTACACATCAAAGCTTCAAAAC
ATAAATCCATTCGTTCATTAAATAGCTCAACAAATTTCTGTTCATTGAACTGTGTTCCAATATAAGAATC
TAACGCGATGCGAGGAAGATTCAGTGTTACAACACCAAGATTATTGCGTCCATCAAGAATTTCATTGCCA
GTCGAATCTTTCCATACGCTCAAGAAACTACGGCAACCCATCGGAGAAACAGGAACAGATGAACCAGTGA
TAGCTTTATTGTTCTTAGCTGAAATAATATCAGGATACATCCTTTTGCTTGCGCACTCTAGAGCAAGCTG
TTTAATATCATAGTTCGGATCGTCTTTATAAAGATTAACACCTTCTTCAACGAACATAACAAGCTTAGGG
AAAATAGGAGTTATCCCATCACGACCAAGACCTTTAATACGATTTTTCAGAATTGCTTTCTGAATCATTC
GTTCAGTCCAGTCAGTTCCCGTACCAAATGTAATTGTTACAAAAGGAGTCTGTCCGTTTGAACTGAATAA
CGTGTTCACTTCATCAATGTGTTCAGTAGAGTTCGCTACTTCTCTACCCGTTTATATGAAACAATGTAAT
TTGGATAATTGTCATCTAAACACAAATCTTTAATACGACTTGGGTGGAGTTTTAAATCTTTAGCAGCATC
CACAAATGATTTATAGATATTGTCATTTATTTTAACATATTCTGGGACGGGATGAATTGTTATTTTTATA
TCTATAACATTTGGATGATTACTAACCTGTTCTTCAGAGCATTTAAGAAATTTTGCAGCTGATTTAAAAC
TTCTAAATTTATTTCCGGATTTTAAAGCTATAGAAACAGTCTTTTTAGCCGTTCTACTACCAATATTATT
TTTAACATGAGCTTCACTTCTACTATTATTTTTATAATGCTCAATCAATTTCTCTCGTATCTTATCTTTA
TGTTTTAAAGATAAGATTTTACCTTTATGGGCATTACTAAGTTTTTGCTTATGTTCTTCTGAATCCGGAT
ATTTGTTAAATTTATATCCACCTATAGATTTATTAAGAATAAATTCGTTATTAAAATATTTCCTAATAAG
CATTTCTTCATGTTTAAGGGCCGATTCATAAGAATCAAAAACTTGAAGAATTATCCACTTAGCTTTATAA
TCTTTAAGCTTTTCTTTAACAAGCTTAGATGACGAATTGTATTCTTTCCAATTTGTATCTTTACCATATA
TAGTTTTGAATTTTTTAAAACCTATATAAAAAGACTTATCAGGAAATCTTACCATATAGGTAAATGCTAC
AGAATTGGCAATTTTTAAGCTTTTTCTTAATTTCCATTTCATCGTTTCACCTCGTATTCATATTTATACG
AGGATAAACAGCTGCATGTCACCATGCAGTTCAGACTATATCTTCAACTCTTAGAGTTGTCTGCCGTTTC
GGGTCGCTTGACCCTACTCCCTTACATTCATCAGGGATAGTCGTTGGGCATTTACAGCTACTGCTGATTT
AGCAACGGATTGTCTCAGTGAGAGTTTCCCGTTTTAGGCAGATTTTACATGAGCTTGACTTACGTTAACT
CATAAGCTTGGAATGCATCGTATACGTCTTTTTCTGTTTTAGATTGAGCATAATTCAACGCATCAGCGAT
TTGCCATTTTTCTGCATCCTCAATATGTTTTGCATAGGTGCGTTTAACATAAGGAGAAAGTACTTTATCT
ACATTCGCAAAAGTCGTTCCGCCGTATTGGTGAGAAGCAACTTGCGCAGTAATTTGTGCCATAATTGCAG
TAGCAACTCCAATTGATTTAGGAGTTTCAATCTGCGCATTACCAAGCTTAAATCCGTTTTCAAGCATTCC
TTTTAAATCTACTAAACAGCAATTAGTAAATGGAAGAGCAGGGGAATAATCAATATCATGCACGTGAATA
ATTCCGCTTTCATGCGCTTTCATAATAAAAGACGGGACCATATTTTTGGCAATGTGTTTAGACACAATAC
CAGCCATAAGGTCCCGTTGAGTTGGAAAAACACGAGAATCTTTATTAGCATTCTCGTTTAAAAGGTCTTT
ATTAGTTTTATGAATTAATCCTTCAATTTCTTTTTCAATTGTCATTTTAAACTCTTTCTAAGCTGCTTCT
TGAATGAAGCTATTAATTGTGTTTTGGTGTCAGATTCATTATATTCAAATCCTCTTTGAAGCATCTCGGC
CATCATTTCCTCTTTTCCTAAACGAGAAAATTCCTTTGATTTATCTCCAACAAAGTTAGGGTGAATATTA
TTTTGGGTGTAATCGGATTTTAAATAAGTAAGTAAATTTTCTAACCATTCAAGATAATCAACACCTTGTC
CCTTTAAGCCAGAACGATTAAATTTATGCTTCATTTGACCTTCTGCAGCATTGCATAGATTACAAAGCAA
TCCACGCACCTTTCCTGCTTTTGGTCCATTTAATTCATGGTCATGGTCGAGGTGATTAGCTTGAACATCA
GGATTTAGTTCTCGTTGGCAAATTAAGCATTTACCGTTTTGTGCATCATAAAATTTCTGTTTTTCTTCTT
TGTATAATTTGCCAGTCAATAACATAATAAACCCTTACCTTAAATAGATAAGGGTATTTATTATTTTCAA
GTATTGTAAAACATTTGATGCAATCGCTTATATTGCTGAATCATTCGGTCAGAAAAAGAAATTTGAGTTT
CAAGCCATTCAATGTACTCTGCGGCAGCTTGCATTAAATTTCCTTCATAGCCATCGTTATTTTCTTGTGC
AGCTAATTTAGCTAATGCGTATGAAATACGTTCACCTTGAAAATCGGCTTTAGGCTTCTGAACAACTTGA
TTAGTTCGCTCTACAACTTCTTCAATTTCGCCATTTTCTACTGATTCAGTATTCCACAAACACCAATACG
TAATTGGCTTATCGTAGATGTTAATAATCTTTCCATCAGAAAGTTCAATTTCAATAATTCCAGTGTCAGG
CTCTATATCATCTTCACATTCTTTTACAAGTTCACGAACTTTAAAGACAGTACCTGCACTAAGTTCCGGC
CAGTAATTACACAGCCCTGTATCAGCACGATTAATTCTAAACCATTTATCTACTGTAATCATGTCCCATC
TCCATATCAATTAAGTCATTTATCGTTGGTTCATTATACACCGTTTCTTCATCAGTGTAAACCGGTTCTT
CCGGCTCTGGCTCTACAGTTTCCCATCTAGCCGCCCACCAAGGTTTTATAGCGTAATCCATTCTCGTACT
GTCTGAGTTACTACACGTTCTCGAAGCTCAACCAGTTCAACAGTAGGAACTGCATAATACCAGTCAGAAT
GGTAAGAACCTGAGCGAGATTCATTTACAGCCACATGTACATTATGTTTTGGACTAAAATAAACACACTG
ACGATATTGGCATTTACCATCTTGCACCCAGTCTTCTATTTCGATAGGTTTAAGATAGTCGGAAAAATCG
AAATCATAGTTTTCCGAAAATGCATCATGGTCTTCAATGATTTCGCTCAGAACTGCATCAACATCTAATT
TATTTTCAATATTCATTTTTCACACCACCAACTGCTTACTTCAAAATCGGGTTCGCCATGTTCCCAGAAC
CAGGGGCCGATAGGAATCCACTTACCTTCATCGGTGACGTCATATTCTTCGGATTTAAGCCCAACGTATC
GCCATTCGACTTCATAAGCGGTACCACCGATTACAACTGTGTTCTCGCCATACGGGTTAGACACAACAAA
GTCTATACCTTCTGCTTTAGCTAACCAAATCATGTATTCATAGAGTTTATATTCCAGGTCGCCTTCTGTT
CCATCGCCTAGAAAAATAATTTGTTCATTTAATTCTATCATTTAAAGTATTCCCGCAATTGGTCAAATCC
ACCAATATGACTTCCATCAGGAGCAAATACCTGAGGCATTGTTAAGCCGATTTGAGTATCACGACCTAGT
TTAGTCAGAAGCTCAGCAATTTTCTCATCATCAAAAACACCTTTTTCCGGCATAATGTTGATAAATTCAA
ACGGCTGTTTCTTCACAGTCAAAAGACGTTTTGCATTATCGCAATACACACATTTATGAATGTTGCTATC
ATAACCATATACTTTAAACATATTATTCCTTAATTCCTAGTACTTGTTTAAAAGTCTCGTCGTAATCAAG
ACTTTGGCCCGTTTGTTCTTTATGCTTGTATATAATATCACTTACTTCTGATAGCATATTTTTATATGAA
CGAGTTAAAGCAGATTTAAGCACGCTGTATCTATCAGGAAATTTACCGTGTTCATTATAATAAGCTATTG
CTAATTCACGGACTGCCTTTTCAGCAGTCTCCATATATTCTTTACGCTTTGTCATTTTCTTCTCGGTCAA
ATCGGTGTTTACAATGGCGACATTTATAACGAAGATTATTAGTCTGCCAATGGACCAATTGCACCTGTTC
AGTTCCACATTCAGGGCAATTAGGAACGTTTTTAGAAGCCCTTTCGCGACGTTCAACCATGACCATTACA
GAATCCCAATTAACAGGAGGGCTATAATCATCACAACCATAAATCTTTCCACGCATTTCCAGATCGTCTT
CTTCACCAGCCATAATAATTTTAATTAGACTAGAATTACTTGAAGCTGCAATGTCTTCTAATAGACGCTT
TTTCATTTCAATACCTCAATAGCATTACGTAAACCATTTGCTTTGGCGGTTAAATCCTTAAGAACTTTAG
TATGCTCTTCAATCTGTTTTTCAACTTCAATCAAACGGGTACTGAGATATTCGCGTTCTTCTTTCATAGT
ATCATTCCCATGATTGGGCTTTGGCGTAGGTTTAAATTTATTAGGGTCCTTTAACTTAATAACAACTTTA
GTTTCAAACAGTGAAAGACCATAATTCGTATTATCATCAAAGGATTCAACTACGTCCATTTTAGACAATA
ACGTACGAAGTTTATAAGTCAAAGAACGAATCGTTTTATTATGTTTATTAGCTTCACATGGTTTCATATA
TGGAAGATTTTTATATATTTCATGCGGAGCACAAACAATAATTAACAATTCAAATGGTCCATTCTGAGAT
GATGGAATGAATTTAGCCGTAACCCAATCTTTTACATTTACATTAATAGTACGATCATCGCCATGACAAA
ACAGTCTACGAGAATGCTTAAAAAGCATATTCACATTATTATTAAATTCGTGTGAAAACTCACTGGCAAA
TTTTTCTTTACTATCTTTAGAAAACCATTCTTCTAAATAAGTTCTTTTCACGTCGTTAATAATATTAAAT
GTAACAACATTATCATTTGATACATTAGTCTCAATGTAGCTTGGATACATAAATTTTTTAACATTATTAA
CAGCTATCACAGAAGATAGTAATCGGGAACGTTTGAACCACTCGAGCAAAGAACCAAGAGAATGTGAATA
CGCATTTTGTGTATCTTTACAAATATGGTTTTCCCATCCAATATAGTCTAAAAAATCACGAAGAATATTA
TTGATTACTTCTCTATGTCCTTTCTGATAATCACGATGTTTAGTAATAAGACTGTCAAAATAATCAATAT
AATGTTTACGAGTTTTCATGTTCTTCTCACTTGGTTAATGATTTATACTCCGAGCCATCCTTGGCTTTAA
ATTTTACTTAATTAACTGCAAAGCTTGTTCTAGACGATCAAGACGATTAACGGATTCTTCCCAAATCTTT
TTAGCCTGCTCATATTCTTTCTGCGCTTTATTAGAAATTTCTAGAACTTCTTTATAAGCTTTTTCTAGTG
CAATAACTTCTGGACGAATTTCTATCGGTTCAGGTGAATCATCAAGACATTCCATTAGTTCCTCAAGGGT
AGTTTCTTCTTTAGGAGTATTCACAATTTCATCACATTTTTGTTGGTAAATTTCTTTACTAGTAGGTGAA
TAAGCACATTTCACTTCACGAAGGCTAATTACCAGTTTATATCCTTCCCAACCGCATATATTCTTAAAAG
GATATGTATGAATATTTTCTACTGTTTCCATACAAAGTAATGCGGTCTCTAATTGACGAGTTATAGTGCT
AGCAATTGAGAAAAATTTATTAATATTCTTTTGGTTAGTTTCTGGTGTAAAAAATCCATAATTCACAAAA
ATAGATACTTTATTTTTATCAAGTTCATATTCTTTTATGATAATCATATCAGAAGCCAAAGGATGAATTT
GACGATAATCGCCATAACATAATTTAGAAGCTGATTTTAGAATTTGCTTCTTGAAAAGTCTGAAATTACT
AATCCAACGACGAGTAAAAATATTCTCAGGGTCTTCTTTATTATTGAGATGATAAGAATTAACATCACCG
AACCAATTATATCCTACTACATCTTTAGTTCGTTTAATTTCTTTCCCAAAATTACCAAGAATATCCCGAT
TAACCAAATATGAAAGAAGACGAGACTCTTTAAAGGTTTTCTTGATAACATCTTTATCTACACGGCTAGA
AATTTTACGAATTTCATGAATAAATTTAGTAGAAGAAACAATACTTGCATCAACACTATTGAGCCACTGA
TTGATAATAGAAAGAACGCTATTTTGGCCAAACATCGGTATAGCTTTATCATCAATAACGCTATTGAATG
ATTTGATATATTCGTTACGAGTCATATTAATCTCCTCAGTAGAAAGTAAGAACATTATACCACATCCTTG
TGGCAAAGTAAACTAGTTCAGTGCATTTAGTGCATTGTTCAGTTTAGAACGTTGCTTTGTCAGATTTTGA
ACCCTTGACTGAGCTTGTTCTAACGCCTTTTCAGCTTCTAGCACTTCATTGGTCGCTTTAACTAATTCAG
CATCTACTGCCTTAAGAGACTTCTCAATCGCATCCGCGTGCCATTTTTCAACAGGTTTAAGACTTGGATT
TTCAACAGGAAGAAAATTCACTTTATTGAATTTCCATGCATCTTTATTACCTGAGCTATACATCCAAAAT
TTAGGATCGTTTGATAAGTAATTAGATAAATTTAAGTTATCTTGCACTTCTTGCTTTTTCTCTTGTGTGA
CTTCGTCCTTTCTCAAAAATTCATATTTAAATGAAACGATCATATCAAGTTCATATGATGTTGTATCTAA
CTTAAATCGTTCAAAACGAGGTAAAATCTTAGATTGAACAGCAGCAACAACATCCATATACTTAAATGCT
TCTGTCAACTGCGATTTAAGGCATTCGCAAATTGAAAGAGAATTTTTTGTATTAGGTTTAAAGCTAATTC
GTGCAGTTCTATTATTTTCTTTTAACGGTCTTACTTCCATCTGAAGAGTATAACCATCAAATTTCAGATT
CTTTAAGTTAATGTCAGAGCCTTTTAATCGACTAGCAGTAGACAAAATTTGCTTTAATTGTCTACGGAAC
AGAGCAATAAATCTCCATTCAATACGATAATGATTTGGATTGAAAAATCCAGATGATAAATCGACCTTAC
TTTGACCAACGCCTTGAACAAATAGAGGATTTTTATAATCAATAGTTTTACAGAAATCGGTGAGCTGTTC
ACGAGCAGTCATTTGAGTAATATACCCTGCTTTACTAAAATTACGCACCCATTCACTGCTATTCGTATAC
TTAAAGTGATGAATAACACGATTAACTTTATCAGGGTCTAAATTATTTTCACACAAAAATGTCATAACAT
CACGAGTATAGCTGGCATTACGAACCATATCTTCAATTTGAGAACGAGTTTTCATGGTGTTCCTTAAGAT
TTAAGTAAATCAACAATTTTAATTAACTTTTCACGCTCAGATTTAGCTTTACTACTCAATCCAGATAGTC
TGAAAATTTCATCATCATATTGTTGAATAGAAATATCAGCTCTTCAATTTGCTTATTAAAATAATCAATT
TGTTCAGAATGTTTTTCGTTACTACGAACTGGTACAGGTTTTGTAGGCAATTTAGTTGAACTGGATTCAT
TCGGGCGATAAATTAAAATGCAATTTGAACCAATCGGGCATTTAGCACCAGATGAATATTCTAACGTTCC
AGCTTCTTTTAAAACTTCAAAAGCTAAACAGAGATGATGCCCCATATTAACATAATCAGTAGAGCGAGCT
CTGATGTAATAATCATCTCCACGAGTGCTAAATTTAAAGCATTTAAGGTCTTCTGTATTATTAGTTTTAA
AAATCATTTGTTTATCTAGACCTTTAGCCAATCGAGCACCTAATGCTAATAATCGTCGTTGATTTTCCCA
CAAATCTGCGATCATTTGGTCAAATGATAATTTCGGCATTAGCCGACCATAAAGGTCATATCCTTTATTA
TAATTGCGAAGGATTTCACTCGCATTAATACGAGACAACGCTCCAATTTTATTAAGGGCTTTCATAATAC
GATTAGATAAACTCATTCCAGACCCGTTTGTTCGCTGTAAATGTTTTTCTAATCCAAATCCAATATCAAC
TTTAAATTTATCTAAAATTTCAGCATGAACATCTCTGTCAATAACATTCAAATCCAAAGTTGGGTTAAAT
CTATGAAAAAATTTATCTGGCTCTCCACGACGTAAAACAGTCCATTCATTCATCATTTTTTTATTAACTA
AAGATTTAATTACTGCGTTGACATTATTAATTACTACTGACATATTTTCCTCACTCAATTTTACCAATTA
CGCGGAATAAGATTGAACAGACTATATAAGTACCACATATAGATTGAACTAATGCCATTCCAAAGAACCA
AACAATATTATCAAACCATGTCTGTTTTACGTCGAAAGGACGTAAACTTACAGTAATATTATCACCTTTT
TCTATTGAAGAATACGTCTCTGGGGAAATATATTCACTAAATCTATAACCGTCTTTGAGTTCATATACAG
CAATAAACGATAAACTAGACCCCTTTCCTTGAGTTCCTGTAAGGGTATTAACTACAGTAACATCATAATC
TTTATAATGCATATAATCATTAATTGCGTAATAACCATATGCAATTACTATACATAAACAACATATCAAT
AAATTCAATCTTTTAATTATCAACTGTTTCATAATAATCTCAATTAAAAGGGCTTAGAACCATTATACCA
TCCTTGGTATAAAGCGGTTATGCGAGTACCGTATTTAACCGTTCTTCAAACTTCCGAAGAGTGTTCTGGC
GTTCAGCTCTTTGCTTTTTGTAAGTTTCAATACGCTCTGAAATGAGAGTGTATCGTTCATTTACTGATTC
TTTCATAAAATCAGGAATTTCTCGAGAAGCTTTAATCTCGTCAAATTTATCAATAACAGCTTGCTCTTCA
GCAATTAAGTTATCATACATCAAAATATCTTTCTTGATGAACTCAATATCTTCTTGAGTTACACGAGATA
ATTTAGATGCTTTATCCTTTTTGTACTGTTCGTTAGTATCACGAGACCAGTGTAATGTACGATTTTTATT
CGTATTCTTGTAAATTTCTACAATACCAATCTCATCGATAATAACGATCCAATTCCAACGGGATTTGTAA
ATTTGTCCTCCATTAACAGTGATTTCACCTCCGATTGAGATGTCATTAAAGAACTTGCTTTGTGCTTCTG
ATTTAAATTTACCATCGTTGTAATTTACCAGGTTGAAAATATCTTTAGCGTTCATTTTGTGTTCCTCCGT
AGTTGATAGTTGTATAGTACCACAGAGGAACGGTCTTGTAAACAACTAAAAGAAACTTCTTTCACAATTT
TTTCCACTGAACCAAGCGCTCACTGCTTTCTTAGTTTCAGGAGCAGTGTTATCCATAAACCATTCAAAGG
CAGCCTTTTTATGATTCTGGAGGGCTTCTCGGGCTTTAATCTGCTCACGGTCTATTAACACTAACATATG
AGCCTTTCTTGTCACCAGGGGCTTCTTATGATTTTTTGAATACTCCCAATCATTTGTCCATCGCATCGTT
GTTGCGAATTGAAATACAGCTTCTTTAATCTTAGTTTCGTAAATTTCACGAGCCTTTGAGTATAACATCA
TTACCTCCATTTACCAGTTTAATTCTAGTCATCTTTTTGATGGCAGTCCATATAATCTATTTCTGAACTG
CCTTTTTGTCTTAGAAGGCCTCTTATGAATTTATTTCAGAAGAGTAACCCGTAGCGATTTCTTCCCAACC
GTTTTTGTCGGTCATAATAAAGTCAGCAAGATAAAGTGCAGTACGCAGTGAAACATTACGTAAACGGTTA
ACATTGACTTTCATCCATGATAATGCTTTATAAGTTTCTTCATCAGAAAGACCACGCTTTTGCATCATGT
CGGTTGAAAGAATAACATCTTCAACCCTGACCATAATTTCTTCATTAGTGTGAACACCCAAATCCAAATA
AACTGAGCGGGACACTAATGCTTGTAAATGTGGAGCAAGTTTAGTACCACGGTCTAATTCGCGGTCAATG
TCAACGTTTGTGATAAAAACAATTGTTCCTTTAAATTCGAGCTCACGCTCAATGCCTTTTTCTTCTAAGT
AAGAAGATGCAGTGCTCCAGCAGACTTTACGGGTCTCTCCAGTGTCCAGAGCAGCTTTCAGAAGATTAAG
AATGTCCATATCAGAGAAAACATCCACATCATCAATCAAAAGGACAGAATTCTCTTCACGATTATTCCAA
AGCTGTTCATAAAGACCGATACCAGAGATTTTACCGTTAATGCTTTTATACTCAATGTATCCAATATCAT
TTGCTTTATTCAAAGCTTTATCTAAAGAATATGTTTTACCAATACCCGCCGCACCAGAGATAATTAATGA
ACGAATATTTCCGTTAATAATACCATTCGTCATCATTCCCATAACATTAAATCTTTTATTAATGCGGGTT
TTCATATCTTCATATGATTCTTTAACTTCTTCAACTTTTACACCATCATATGAAATGTCTGATTTGTAAA
CCCAAACACCGCGACGTTTACCGTCAATTTCAACAAAAACTTTACCATCTCCTTGTGCATCTACCGGAGC
ATTATCAGGGAACCATTCACCTAAGAGCTCAAAAGTTCCAGAGATTTCTTTACCGAAGTACATACCCTTA
TTGATAGTTACAGTTTTCATTTTATTCTCCAAATCCGTATCAGTTGATAGTTGTATAGTACCATAAAGCT
TTATGCTTGTAAACCGTTTTGTGAAAAAATTTTGAAATAAAAAAGGGAGCCCGAAGGCTCCCTATCATTT
ATAATAACTTCGATGGTTTTCAAGATAAACCCTCTCAAGGAAGTCATCCCAGAAACTCATGTCTACTTTT
TGCTGCATACCGTTCTTAGAAGCTTCAGTAGATGCTGCTTCTACTTGATCGACCACATCTTCCAAAAACT
CTTGAACGGTTTTAAATGGATGCTTACCCAACTTCACGTCGAGAATAAATGGAGCATCTTGGAGTGGATA
AACCAAGTCACCAGTTTTGTAAATTTCCAATAGTTGAAGTCCACCACGACAAGCATGGCTCAGAGCTTTC
CAGTCAATGCCTTCATTGGCTTCGGCCTTACGAGCACGTTCGCCGTATTCAGCATCTAATTTGTTCAGTG
ACTGCTTAAGCTCAATAAGAGAAAGCGTTGTCTGATATTTACGACCCAACACTGTGTAAAACGTTTGTGG
GCCTGTTTTCTCATGATTATGGAACACCCATTCACAGAATTCGTTTTCTGGAAGACGATGCTTAATATCT
TCAACTTTAGTACGACGCTGCTTAATGGAACCATCTTCTTGGTAATCAACCCATTGCTCAGGGATTTGAT
TAACTACTTTCAATACATCGCGTAATGCAGCCAAACGAGAACCCTTGACGCCGTATTTAGAAGCTTGCTT
GCGGACATATCCTAAATATGATTTCATGTTAGTCGTATAAAAACGAGAACGGTTGTCTTGAATAAACTTC
CAMACATCAGGCAAATCGGATTTAACCACTAGTTCAGGTGGAGTGTGAAGCATATCCAATGCTACAGGTT
CACCATCTGCTGCTAATTTAAAGAAATACTTAAGACTATACAATTCGTGGTCAATATTATCTTTAGTGTT
TTTAGATGATGTGTTGTTGGTATTTTTACTCATGTGCTCTTTGACGTTTCCAATAAGAATATCGCGAGCA
GGAGGAACAAAGATTTCTTTAAAATCTACATCAGATTCTGGAGTAGAAGTTCCATAAAGATGACTACCAA
AATAAGACTTAACTACAGTTTTCATTATTAGACCTTTCATAATCTTCATTAAATTGTACAATCAATCGAT
GATAATTAGATTTTGGATATCCTAATTTACTTATATAAGTTCCGAATGACCCACGTTTAGGTCTATTTAA
TTTAATCCATAACTTATAAAGTAAGTCATAGTCTTGCCAATGTTTACCAGTTCTTTGTCTAATTTTAGAT
GAATTTGATTGTTTCTTTTTAGCTTCAAAATTATTCAAAGATTTTTTGACTGCAGCAGAGTGTTTTTCTT
TAACTCCCGGTCGTTTAAAAGCAGCTTTCACCCCTTTAGATATTTTTTCTTTAACTTCCGGTTTGTTTTG
TGCTTCTAACTGGGATTCTGAAATTTTAGCCCTTATTTCAGGAAGACTTAATGTTATCGAACGTTTTTCT
CTGTATTCATTAGACTTCCACATTTCTTTCATTGTATTAGAATGTATTTTTGAAAATTTTTCTCTAACTA
ACTGATATCCTCTTGAAGTTAATTTGAGATTTCTTCCTAAAGAATCTTCTCCAAAATTATAAAATGACCA
CCATGCATAAATTAATCCAGGCGAATTGTAATGAATTTTAGCTAAAAGCCAATGGGCTATAAAATGCTCT
CTAGCTGTTAATAAAACTAGATTATCAGAATCATCATTACCACCAATACAAGATGGAATAATATGATGCT
TTTCCGTATAAAAATTTAATTTAGATTTATCTAATTTTCTGAGTTTTCCTTTCTTAATTAAATTATTATA
TACTTTAGTATAATTCATTGGTTCTTCGTCTCATTTAATTTTGCTTTGCATTTATAACACATGTCTGTTC
CACTTGAAATAAACATATTTCCTCACTTTGAAATCATAGTTGGAATAACAGAATCAAGATAAGTCTTTAG
TGCAATAGCTTCCTCTTTCTTTAATGTAATGATATGTGATCGAAAATCATCAATTTGACGAATAGATACT
ACATCTCCCTCTTCATAGCATTTTGAAACATTTAAAATAGTTTCATCATTCTGATTACAGGAGTTAGTAA
TAATAGCATTACATTTTTTAAACCATTTTAAATTATGTTTTCTTTTAGTAGAATCATAAAAATATTTAAT
GTTAGTTATAAAATTATCCCAATTATTAATTGATAAGCACATTGACTCGCTTTTAATATTAAATCCTGGG
CATGAAGAATAAAAATGAATTTTATGCTCATCATTAATGCTTACAATTTTATCAGTGTAAGCATATTCAA
TTTGGGTTAAACGAACAATTTCGCTAGGCGTAAAATATAACATGTCATCTTCTTGCGTCAAACGATACAT
GTTATTTACTTTTTCTATAGCCAATTCACCAAAAAGTGGACTAACTTCTAATACTAGTCGTTTCGCCTCG
CTCATCATTACATACTCCTCTGAATCATATTAATAATGTTATTCACCAGATTATAAGTAAACATTGGGTA
ATTATATTGAATCATCACATATATAACAAACAAAACTTTCATTCTCTTCTCCTTGGCAGTTGACAAGATT
ACTATACCATAATCTTGTCAACTTGTAAACCATTAAATGACGTTTTCGATAAAATTTTGAAGCTTTGTAT
GAGCATCAACCATGATTTTCATTTCTTCCTTGGAAAAGCTGTGCCTCTTTCCCGAGAAGAACATTCATAG
TCAGAAACTGCATTAGCATATTCTTCAATTAGCTTCATTAAAAACATCTGTTTTTCAGTTTTCATTATTC
CACCTAATCATTTCAAGATATTGAACTAACTTAGCTTTGGATTTATCCAAATCCCTTTTAGCTGCTTCTA
TACCGTCGTATGAATATCCTTCACAATGCTCAACTGCTAATTGATATGAATCTATTTCAATATCATGCGC
TAATTTAATGATTTTTTCAAACTGTTCGCGTGTTAGCATACTTAAACTCTCGTATTATGATCGATAATTT
CATCAAGAAACATATCTAACGCTTCTACAGCATTATTAACTTTAGCTTCAAATTCTTCAATACCTGATAA
GGCAAAGTTAATGCGTTCCTCATCTGCTTTACGAATTAAAGCTACCAATTCCTTAATTTTATCCGCTTGT
TCGATACTAATCATTATTCCACCACATATGAAAGAGAGAATATTGCACACGCCATGTGAGTTGCAACTTC
ATCACACATATTATAACGTTTCTTAAGAAGTTCTACAAGTTCTTCACTAGTAACTTCATCCATGTCGACG
AAAAAATCACCATTAATGATGACGTAGATATTTCCTTCTTGATTGAGTGCTTCAATTTTCATGATGTTCT
CCTCTTTATCCGATGGTTGTATAGTACCACAGCTCAAACGGAAAGTAAACCGGTAAAATGAAAAAAAGTC
TCCCGAAGGAGACTAATGTTATTCGAGGGAAAGAAGATACTTACTCTGGTAAAACATTCCAGTAATATCA
TCTATCGTGCTTTGGATGGCTGGAGGCATTTCTTTATAAATGCTGTTAGATTGGTCTAGTATGCGATCAA
TCATTTTAATTGTGTCGGTAGGAAGTTTACTGGCATCTGGAATTGAAGGCGTGTATTTTCGACCAGAATA
CCCCAAATATTGCTCACCAAATTTATCAATCAAATCTGGCAACTCGGAAAAAATAAAATCGTATGCTTTG
TGTCTAGCATAACTTTTAGTTTCAAAATGTGCAGAATGAAAATAAGCTTGTGCAGCCATTAATAAACCTA
AGTATTCATCTGCCTTTGAAGGTTTTCCACTTTGTGAAAAGTCGCTGAATTTCATTCAGTCTCCAATTTA
ATGTTCATAATTCTAGCGTATGATTGTGCCATCTCCGCGCCTCGCTCTATACATTCAAAATCAGAAGAGC
ACGGGTCATTTTTATAGGTCGTTCTCATAAAACTATAGAATTGTTCAGACGATTCTACGCTTTTATTTTC
AAAAAGCATATAAACGTGCCTAATACCAGATTCCATAAATTTATCAAAATGAGGATCGACATTCGCTTCA
ATCGATGGAGATAAAACAAATGACAATCCTAGCATGGCAAAAAGTGCTGTTGCTTTTAAGGCCATAAAGG
CCTCCTATCATTTTTGTCCTGTATTTACTTTGTGCCGATGCACGGCCTTAACTTTATCAAGGTATTTTTC
AAAATTTCGCAATCTAGTATAGTCTGCCGGAGATTGGTTGAGTGATACTTCTCGACGCAAAGCTGAAATG
ATATTTCCAACTTCCCTACGAATTTCATCTAATTGAAGAACAGTAAGATTGCGAAGTTGCTTTTCAGTTA
ATTGTAGCATATATACCCCTTTAGTTAGATAAACCTATTTATAACTTTTGCACTAACCGAGCTTTTTAGT
TAATTCATTCCAATGTTTTCTACACAAAGAAACATAAATTTCATCACCAATACAAATTTGATTACCTTCT
TTAACTGGTGTTCCATCTTCCATTAATCGAGCTGTCATAATCGCTTTTTTACCACAATGACAAACTGCTT
TTAGTTCAATAAGTTTATCTGCAATCGCTAAAAGTTCTTTAGAACCTTCAAATAATTTTCCAGCGAAATC
AGTCCTTAGCCCATAAGCCATAACAGGAACATTATATGTATCAACAATTCGGCTCAATTGATGCACCTGT
TCAGTTTTTAAAAACTGAGCTTCATCTACAAATACGCAATGAATATCTTTTTGTGCTTCAGCCCATTTAT
AGAACTCGAAAATATCCATATCATCTGTAATAATATTCGCTTCCTGCTTAATTCCAATGCGAGAAACGAC
TTCACAGACAGAATCGCGAGTATCAATAGCAGGCTTAAGAACTAATACACTCATTCCACGTTCTTTATAA
TTATGTGCAGCAATCAAAAGAGAAGCAGATTTTCCAGCATTCATTGCTGCATAAGTAAAAATTAAACTCG
CCATCTTAGTCCTTAGTTAAATTTTCTAAATATGTTTCTAAATCATTTTCAGCTTTATCGATAGATTTTA
CTAATTCGTAATATGTTTCGGCATCTCCATATTCAGAAGATATTTCAAAAGACAAATCCTTTTCTAAACT
AATAATTTCACCAACTAAAAATAATATTTCGTTCTTTTGTTCGCGAGTAATCATAAGGAATTTATATAAT
CAATGAGTTCTTGTTCTTTATTATCGAATTCTTTAGAAAGTTCTTCGTACTCGTTTGCGCTAAAAGGACC
GCATTCATTACAAACTTTTTCCAATTCACTATTTTTATCCATAACTTCGTGGATAAGAGAAAAGAGTGTG
TCTTTTTGTTCTTTGCTTAAACTCATAACCATGTCACCTTTAAGCAGTATTCTTCTACATGCTGTTTACG
ACCTTTCTTATCAATAAAGGTATATTCAACGAATGTTCCAATGTAGTCTTTATCTACATCATGTGGACTA
TTAATTGGACATTTAGTGCGACAAATGCGTTCCCATTGACGAATAATTACTGCCTTATTCTTTGGGTCAT
ATGGATGTGGATAATGTATATTCATAATAATGGTTCCCAATCAACAATCACAATTTCTAATTTAGAGGAA
TATGTATCTAAAATCCCCTCAATAATATCCCAGTTCCCTTTACCTATGCCTGCACCAATCCTAGGCATAT
AGATTGTAGGTTTAATCAGTTTATTTTCACCAAACTCATTTAATTCTAACATACAATTCATTAAAGCGGA
ATACTCAAAATTTGGCCCTGGTTGAAATTGAGTATAAAGATTGAAGCAGTAAGCTTTATGAGTCCTAAAG
TATTTTTCATAGACTGAGTAAGAACCGAGTTTAGTTACATCACCCCATTCAGTCTGTAATTTATCAGCTT
CCAAAATTTTAGGGAAAGCTTTGGTTAATTGACCCGCTACGCCTGAACCCATAGTATGAAAACAATTACA
TCCATGTGCAATATTTTTACCTTCAGCGAAAAGGGCGACAATATCGCCCTTGATATATTTTACAATCATC
TAGTACTCAATCCTCGATTATAAGAATCTACCAAACGGTCAACCATTGAATGACAAGCGGCTTTATCTTT
CTCCTCCGCAACTGAACATTCTAAGGTATTCCACTTTTTAGCATATCGTTTTAACAATGTATCGTTTTTG
TATCTGCTTGATTTATCTCTTTCTCCGTCTTTATATGCATATATTAATTTCTGTGCAAATTCAGCTTGGC
ATGCCTTATTTTTCCCACAATAATCTGCCGCAGTGCGGTTTACATATTCTCTAATTTCAGTATATGATGT
ATCTGCTGACGCAGAAGCAGAAAATGAAATTAATCCTATACATAAAACCAAAATTTTAGTCATTTACTAT
TTCCAAAAGTTTATTATTTTTAAGGTAATTAGCCTTTTCTAGGACTTCAGAAGCATATTTAGAACCTGCT
TTAACATTCCATCCCGAATTATAAGAGGATATTGCTTTTCTTATATCGCCCTTATGTATATTTAACCAAT
AAGAAAGTTCAATGTACGCCCAGGAAGCTGAATTGGATCGTTTATTCAACATTCTTTTTATTTCAGCATC
GGTCATATTATAACCAAGTTCCTTAACTCTTGCTCGCATAGTAGGCAAATAATTTTGGAACATTCCGTAG
GCGTGATGCTTTGGTTTAGATTTTAAATTAACTCCGCCAGAGCTTTCTTGCCATAAAATGGCAGCCATTA
TATGACCTAATCCGCTCTTGTGGATATTTTTGTGTGTTTTATATTTTCCATCCTTAGAAAATTGTTCCCC
GAATTGATACGCGTAACGCATGTTATCGAGTTGGACATTACTGAAAGTATGCTCGGAGCTATGTGCCATC
ATTGAAATGGCCAATAGACCAGCGAGTAGTGCTTTTCTCATGCTTACCTCATTGAGTTTTAATTACTGCT
TTAGAAGCCTTTCCTGGTAAACGACGACTGTTGATAATTGCCATCCTACATTGAAGTGACGGGTCTTTGA
ACTTCTCGTTAGGTTTACAAACTGTAAATCCAAGCCAAAGATTTCCATCTGTGATTTCTAAACGTCCAGG
ACGATATTCAACCCCATCAATAAAATCCTCGTCAATGTCAGGACGCGGAGGCATACTCAGGAATTCATTA
ACTTCTAAAACATGGTCTTTTATTTTATGGAATAATTCAAAAACGTATGTCTCATCAATCTCCCGTTGAA
TTGCGCGATCAAGAAGATGTTGAGAATATTTTAGATGAAACGATGAGACTCCTGCTGCTTTTGATGCCTC
ACGAATCTCATTGTTAATTTGACGAAACTCCGACTCAAAGTGACGACGAAGCTTATTTCGACGGATAAAA
ACTTCTGTATTGATAGTCATGTTATTCTCCTCTTAACTGATAGAAAAATTATACCACAGTCAAGAGGAAA
AGTAAACAGTTATTCTTTAAATCTAATCAATTTATTCATAGACTTTGAAACTTCGGCACGAACCTCATGT
AGATTTTTGAGCTGTTCAAGACGCTGCGTATAGTAAGCAATTTCATCTTCTTCGAGACAGTCCTGCGAAT
CTTCTTTAAGATAACGTGCATAGTCCTGGAAAGCGTTACGGACTACTTCCTGGAAGTCATCAAGACTTTG
AATTTTCTTAGGAGCAACAGATACACGACGAGGGGCAGTATAATACTCATAACCAAACCCTGCGCTTAAT
TGAGCCATTAGTATTTTTCCTCTGGTTGGAACACTGCACGACAAGCCCACATACTGGCTTCTTTGAGTTT
CGTTTTAGCAATAGTTAACTGATCGAGACTTTCAGCATAATTCTTCGCGAATTCACAGTCTTCGCAATTA
TCTAGTGCTTCCCAGAATTCATCATATAAAGCATCAAAGATAAGTCCTAAACGAACTTCAGCGTCTTTAA
TAGCATTTACTTTACCGATTTTCTCTTCAGTATGTGGTTTATAACCCTTAATATCTTCAATCATATTTGA
CTTCCTCACCAGTACATAATACGTATTCAACTAAACGAATAGGTTCATGAATGCCATAGCCTTGAACAGA
AATTTCTGTCGTAGGATAAATTCCATCAATATCACCCATATTCCACGCTTCATTAAATTGCTGTTCGCCT
GAGTTACTAAACCACTCGCGAAAGCATTTAGCACATCTTCAGAACCTTCAATAATTATCTTTGCCATTAC
AAACTTTCAGTAAAGGTACGAGCGATAACGTCGCGCTGCTGTTCCGGAGTCAGAGAGTTAAAGCGAACTG
CATAACCGGATACACGGATGGTCAGCTGCGGATATTTTTCCGGATGCTTAACTGCATCTTCCAGAGTTTC
ATGACGCAGAACGTTAACGTTCAGGTGTTGACCACCTTCAATTTTAACTGTAGGTTGTGGCTCAATTTCA
ATTTCACGGGCATGCAAACCATAGAAAATTTCTGGGTCTACAAAAGAGTCCTCTTTAAAGGTTTTAGAGA
CAATAATTCGTGCTTGAATACCATCTTCAAAATAAATAGTACCTTTATGTGTGCCTTCAAGAATTTGATA
TGCTTTCATATAAACCTCAATTAGAAAATAAATTTATCCAAGATTGTTCTTTAATTAAAAATGGCTCAGA
ATCATATGCCATTAAACTTTGCGTAATTAATCCTTTAAAAGGTCCATCAATAAATTCCATGGTAAAATAT
GGAATTTTATTCATTAGCCGTGCATTAGGAGCAGTGCACAAAACTCTGCATCCTTTGAATACGCCTTTTT
GTAATTTGTATTGCTTGGGATAAAATTCGCTCAAAATGTTATTTTTTGCCAAAATTTCAAAATGATTCAC
CAATTTATTTTTAATAGTTTTTGGCGAAAAATAAAGATATTCGAAAAGCTGAGTGTCTGTCATCATTGCA
TTCCGATTACGAAAAACTGTGGACGAGTAATACCACCAATGCAACATTTACTATTACAGCAGTAGTGTAC
GGTGTCAATATGGACACTATAAATCTTATCCATATCAGGAGATTTGACAGGCTCATCAATTATATACAAA
ATTCGCGAAAGCTTTAAACCTCTGAACTTGCTTCCTTTATTACCAATAAAACTGCGTACAGAATCAGTAA
ATAAACGAAAACGTATATCATCATTAGAATAACGCGAAAATTCCTTTTTGATGTTATTTGCAGAAATTTT
AGCATAAGCTGAAGTATTAGAAAGAACAATAACTGTTCCGCCATCATACAACCAATTAGCAGCAAAGTTA
GTCACAGCAATTGATTTACCGGATTGACGTCCACCATCTAGTCGAAGTGTACAATACTGTTTAAGTAAGT
CTTCAAATGGCGGGATATATTCGTTTTTACAAATTTCTTCTACTCTAGCATCAGAATGGTGTGTAAAAGC
ATTCATCAGGGATAGATAAGGACCAGTTAAAAATGTTCTCATTTCTTCTCTATAAGCTCTATAAGTTTGG
GCCATTCCGTGGCACATGAATTGTCCATTTCTGTATTTACCCATTACCGCGCTTGGGCTCGACCTTATTA
CAGGTTGGCGGGAATCCCTCATATAATCATGAGGTCCAGGTTGTTCCCTTATGCATAAATCGCCTTACCG
TAGTATTTGTACCAAGTAGGACGTTGTGCAATTTTTTCATCTAAACGAGCTTGTGATATAGCAATAGAAG
CTTCATGGGGAATATAATCACCACGGAATTCCTGAGGAATATCACTAATATCCTGGACTGTAGTATCCTT
GATATTAAAACCACGTTTTAAACATTCAGCTATAAGCTCAATTTGACGTTTACGTAAGAACTCGAGCTTA
TCGTAAAAGAATGTAACATGACCTGCGCCAAGGATAAAAGTAGGACTGATTTTAAAATCACGAACACGTT
TACCGTTAGCAACATGCTTACGAACTGCACCAAAAACACGCGGCAATTCACGATATTCAGCCATTAAGTG
TTGGTCAGCCAATTCAGATACTAAAGTAAGGTTGATACGAGTCATTTTAGTGTTCTCCTGTAGTTGATAG
GTCTATAGTATCATACCTACAGGAGATGTAAACTGTTATTTATCTTTAATTGCTTTAGCTGCTTCGATAG
CCGCTTGCTGAAGGTCATCCATAGACATGCCGAACTTAGAAGCAAAGTTATCAATTTTCTTTTCGACGGC
ATTCAAAGGCTTGGCCTGTTTACCTTCATTAGCGCCAGGAAGAGCGAGAATACGCTCTCTGTCAATATAA
AGGCTTACAAGTTTCTTACGGTCTTTATCGGGCAAATCATGAAATGAATGGGCCTTTTTATTTACAGCGG
CTTCTAATTTACCTGCGCCCACTCGCGCTTCGGCAATAAATTCTTGATATGTTTTCATATGTTTCCTTTA
AATGTAAATATTTTTATTATTCTATCCTAGAATTGTGATAATATATTCACAATTCTAGGAGTTGTAAACT
GCTTTTATTTAAGCGTCCCAAGTATAAGCTTTATTAAGAATTACCACGGGCTGCATTAGCAACGGCGTAA
GCGTACTGAATATTAGCGTCTTTAAACTTACCTTTAGAGGTATCTATTTCTGCCTTAAAGCCGCCTTTAG
TCATAACATCGGCAAATTCTTTACGGAAAGCCATTGCATCAAGACCTTTCCATGATGATTTATGCTTGGC
AAATTCAAGACCTGCGAAGTTGACAGCTTTAGCCAATTTATTATCAATGACCCATTTACCGGCTTTAGGC
ACAAACTTCGGGCCTTTCTGTTTAGAGAACAGTTTTAAATTCCAGCGGCGAAGGTCTGCATCAGCTTTAA
CAAATTCTGAGTCTAAGTTACTAGCAACAACATCTTCAATATGAGCAAATTTAAGTCCGTCAACTTCAAT
ATTTAAATCGGTACGCCAGCGAAGTCCTTCCCAAGCGAAGGCTTTAAAGTCGGAAGCCTTTGTAGCTAAG
TACCGTTCAATAGGAGCTGTTTTAGGGTCAAAGCCGTTTCCTGATCGGTAGGTCCACTCATCTTTGTTAA
TGCCTTTGGCCTTTACTACAGAAGCTTCGGCAATAAATTCTTGATATGTTTTCATATGTTTCCTTTAAAT
ATTTTAATTAGTAATTGTCTATTCAAGTAATTGTGAATATACTATCACAATTCCAAGAGAAAGTAAACAG
CTTTATAGATTTTTATACGCGTCCCAAGTGCCAGTTCTAAACGTTGTAATGACTCGTTTTGCGCGATTAG
GTGTTTGATTATACCATATACTTTTAGCTAAGTTAACTGCTGCTTCATCCCAGCGTTTTTGTTGAAGCAT
ACGTAAAGAGTTAGTAAATCCTGCCACACCGGTTTCTCCCATTTGGAAAACCATATTAATCAATGCACAG
CGACGAACCGCATCAAGAGAATCATAAACCGGTTTTAATTTAGCATTTCTCAGAATTCCGCGAACAGCAG
CATCAACATCCTGATTAAAGAGTTTTTCAGCCTCATCTTTTGTAATTACACCATTGCAATTACGCCCAAT
AGCTTTATCTAATTCAGATTTAGCAGCATTAAGTGATGGACTTTTTGTAAGCAAATGACCGATGCCAATA
GTGTAATAGCCTTCTGTGTCTTTATAGATTTTAAGTCTAAGACGTTCATCTATACGTAACATTTCAAATA
TATTCATAATACCTCCTAAGTATTTATAGAAGGTATTTATAAAATTAAAAGAGGTTGTTCATTATTCGGT
AAAGTGAAGGACCCATCACATATTGCCACTGAGTACGAGGAATAAGAGCAAAAGCGTCCATCTCTGGAAT
CATAACGCCATCTTTATTTTCAAAATAAGACTCGCAACGGCAATTTCTGAACATCTCATGCTCTACTGGA
ATCGTGTAATAAAATAACTGTAGGTCTTTATTACTAGAATATTTAAATACACCTAGGTCTTCTAGAAGGT
CTGGATTATAATTGCTAAAACCAGTCTCTTCTAAACATTCTCTTCGTGCTGCATCTAATGCGCTTAAATC
AGAATTTTCTACACGGCCCTTTGGAATATCCCAACGATGTGCCATCATTCCAGTCTTACGAGAACCAGTA
ACCCGACCCATAAATAAATCTTTATCTTCTGTCATAAAGATAATACCAGCTGATAATGTTTTCATTTTAA
TTTCCTGCATTCAGTGATAAAGTTATTTAAATTTTGAGCATATTTCTTTTCATCAAAAATCTTTTGCTGT
CTGCGTAACCGCCATGGCATTTCAATGAACATACGCCATATCCCTAGATAATACCGCTGCTGTAAAAATA
TTAACAAGTATAGTTAAAAGAATCCAATCGCCTATTCTGTCCATTGGATTTTTATAAAAAAGTAAAATAC
GAATGATGATATAGGAAGACTAATGATATACCACAGAAGAACCTTCTTATCTGTGAACCAATCAGCATTC
GTTAACTTAGCGCGACCATTTTGAATACACACGAATTTATCATCTGTTACAGTAAATGGCTTAGCTGCTT
GATATCCCATTCTAAACTCCCTAATTAATCGTTTCTTTGTATCETCGGAACAACCATTCCAATCAACTCT
ATCAACTGGAATGCCATCATCCCCATCATCTAAATCATACCAGCGAGTTTTTAAAATCATTTAATTTTCC
TGCAATCAATCACAAACTCTTTCATTGATTCATTTTCAATATAAGACATGTAGCTATTATATTCTTTTAA
TTGTATTTTGTAATCCTTTTTTCTTTGCCAATTTATTTTAAAATTATCATAATGAAAATATAAAATGATA
CCAAAGAATGAAAACAATGAAATAATTTTAGTATAACAAAGCTCGCTCCAAACTTCTATTATATCTACTG
TACCACTGATTTTTAAAATAAAACAGTCAATTAATAGTCCAATAAGACTACCTGTAAGAGCTGCAGCCAA
CGCCACAGCAAAAATTAAAAATGACTCAGAAAACGAATATTTGACTTTATTTAGCTTTGGCTTTTGCATC
GTGATTCCTTAACAAATTTCATAATTTCATTAAATTCATACTCAGCAAGTTTAAGCTGGTGTTCCTTTTT
AATCTTTTTGCACTGGGCTTTCCAATCACGTACGCGTTTACGATAATGTCTTCCTTGATACCAGTATCCT
ATCCAATTTACGGGTACTAATAAAAATGGAACTACCAATGGAAGAGTTAGCATTAACATAATTATTACGC
CAGAATCAATATCAGTCATAACATCTAAAACACCTCCAATAATCAATAGAATTACAAATGATATAGCTAT
CACAGGACCTATTAATACATCAGTAGAAATTATCTGGCGCTTTAATTCATACTTCAAAGGTTTACTTGGA
AGGTATAGTGATGGCTTTGACATATTCTCTACATTCCTTAACAAATTTTTCTAGTAATAAATCACTTTCA
AAATTAGGATTTTCCACTAATTTATCAAAAAGATCATCAACAATATTCAAGATATTTCTTTTACTAAGAA
TACGTTTATTTTCATGCTTCGTTTCAGAATCAACTATAAGAGTAAAGAAATATTTCTTTCCCTGAAATTT
TACCGTAGTATCAATATAAAATAAATTTGACTTTTGTAAATTACGTTTAAACCATGCGTCACTTAAACTA
TAAACACCGAGATAATCAAAATCGTCGTTTAAATAACAAACTGACCATTCAGGAGAAATGAAATCAGTAA
ATTCAACATCAAAATCACATGTCAATGAATGAATTGATTCAATACTGTTAATAAGTATTCCAGGACGTAT
TAAAGACTTTTTACCTCTGGAAAATCTTCCAGAAAGACTTTCATCAGTTTCATATGAAGAACCCCAATAA
TAATTACGTCCTTTTGCCATATGTTTAAGAGCATTTAGTAATTGGTCTGGAACATCAACGTGTCTTTGGA
ACTCTTCAAACATTGAATTGAAATCACTTTGCATTTTCATTCCTATTTACTCCAAGTAATAGGGGCCGAA
GCCCCTTATCATTATTTCAGAGAATTAATATATTCCTGAACATCGGCAGAGGTAGTTTCAACCCCAGAAA
TATTACCATTAAAGGTTTCAACTCGAGCAAGAGTATCTTCAATATCAACCTTAGTCAGTGCTGCAATTTC
AACTACATCATCAGCAGTACTAATTCCAAGGGCATTTGCTGCACGAGTTTCACGGATATATTCCAATTTA
ACTGCAAGTTCTTGGCGAGCATCATCTAACTCAACTACTTTCTTGGCGATTTCAATTCGCATTTCAGCAT
AACCATCAGCTTTAGTAGTCAGCTGTTCAGCTGTTCGACGATATAGCAAGCCGAGTTTAGCATGCATTGT
TACATCTTGACCTTCGGAAAGAAGCTTGCGAATTTCACGCTCTTTTGATTCGGCCTGTTTATTCTTTTCA
ACAATAAGTTCACGAATACGTTTTTCTTCATTAATAGATTTAACAGAAGCAGTTTTTAGGTCTTTAATTT
TATCAAGTAGTTTTGCTGCTGCAGCAGTATACTGTTCTTCAACAGATAGATTTTTAGCCATTGCAGAACC
AAGTTTAGTGCGAATAAACTCAACAATTTTCTTCAGTGTGTTCATAGTATTTCCETAGGTTGGTATAATT
AGATAATATAATATCACGTTTCTAATAGATTGTAAACTTATTCTTCGTCTAGCTCGTCGATAAAGGCGTT
GATGGCCTCGATAATGGCATCATTGATAGCCAATAAAATAAAATCATCGTCAGTACCTTTAGAAGATTCT
AAAGCATTGATATATGCTTGGTTGACGAGTTCCCAAGCCTTTTTAAAATAAGGAGCTTCATCATCAGGAC
AAATATCCCGCACGCCTTCAAAGATACGTTTGGCATAATCTAACACCCATTGTACAGGCATGTTTTGAGA
ACGTTCGTTAAACTCTTTAAAGTCCTTAGATTCAAAAAGCTCTTCAGGATAATTATTTCTATTACAAAAA
GCTTTACTAAAGTTACGTTTCATAATGTTTTCCTCATTTGTATAGGCTCATAATATCTCAATCATSAGCC
TATGTAAACTTATTTCATATTATTGAAATATTCTTCTGCGATTTCGTCGTTATCATGGTAAACTTTAGAA
GACAGTTTAACATAACTTTCAGCAGTGAACATGTTAATCACAACCTTTACAGTATACCACTGACCGTCTT
CATTACCCATTACTGCGTAAGTTTCAAACATCGGATGGTCAGGACCGATAACTTTAATATCATTCACCGT
ACGACCGAAATCTTCTGAAACACATTTCATAAAGAAGTTGAAAAGTTCACCGTAATTATCCATTTTATTC
TCCAAGTTATTTTCTGTATCAGTAGTTGATAGTTGTATAGTACCATGGAAGAACAAGGATGTAAACAGTT
TTGTGAAAAAATTTTTAAAAAGTTTTAGGGAATTCTAGGGCGGAGAGGGGCAATTAAAAGATAGGATAAT
ATATTATAAAGGGTATAAACTAAATGATGCCTAGAGAGGTCTGGAAAGGCTTAGATACCAAAAAGCCCCA
ACCTTTCGGTCGGGGCTAACCGTTGCGGCAACCTTGTCGGGGTTCCACCTGCCAAGGCAAGTGTTTGTAC
GAAACGCCGGGATTCGAACCCGGTTATTAAGTAGTTGACGCTACTCAATATTTTTAAAAGGCCATATCTC
AACCATATCCGAACGTTCCGTCAAAAACGCTACTCGGCTTACGGCAAAGATATTTCCTCGAATCGATAAT
TTGGTGCGCCGTTTCTGCTGTGATGTAAGAGGGCATCAATAAACGCAAAGATTATTAACGCAATTCCTTA
CTCAGGGAACCATCAGTCCGACGACTTACCGGTAGCGACCCGGTTTCTCATTTGGTATCCCGCCCTGGGA
TCGAACCAGGACCGCAAACTTAGAAGGATCGTATGCTATCCATTACACCAGCGGGACGTAATTTAAAATT
TCATTTTTCGACCTTTAAACCATCCTTCTGGAATTGGGTCAGTTTTCTTAATACGTTTAGAAACTTTTTC
ATCTAATGAATGAATCCACATCATACCGAATTGGGAATTCTTTTCACCTTTCTGGTGATTATTTTTGGCG
TGAGATTCTTTCATTTTATTAATAGTTTCAGGAGTATGATGCTTATTTAGAAATCTGCTATTATTTAAAA
ATTTTTCCCTGTATTCAGGAGTTGACCACAAACGTTTAAATACATTTGAACCAATTTTACGATATTTTTC
TTGAAGTAAAATATCATTTTCAAAACGTGACTTAAACGATTTAGCTCCTTTTAAGCTAGCATCTTTCTTC
TGGTTTAGCATTCCAGGAATATTTACATGATCCCATCCACCTTCACCGCCAAGTTTTAAATTATACACAT
CTGGTCTATTTAAAAACTCTTCTGTGACAATATTTTTCTCGGCTTCAAGCATAGATTCTTTATCGTCAAA
ATACTCTAATATTTCTTTAGAAAAATTTTCTATACCATATTTATCTTGGGCTCTTTTTAATAATTTACCA
GAACCCATATATCCATCATCTAAATTTTCGGTAGAATGCACACCAATATAAATTTTATTATTAATTTTAT
TTGTTATTTTATAAGTGTAATAGAACATAAATATCTCCTATTTCTAAGAGTATTTATGTTCTCAAAATAT
GACCCAGACCAGATTTGAACTGGTAACCTTTCCCTTATGAGGGGACTGCTGCTAACCATTGAGCTACAGG
GCCTTGGTGCTGATTGACGGAATCGAACCGCCGACATCCTCATTACAAGTGAGGTGCTCTACCTACTGAG
CTAAATCAGCAAAATTACGGAGGCGATAGGATTTGAACCTATGAGTCGCCGGAGCGACTGCCGGTTTTCA
AGACCGGTGCATTAAACCACTCTGCCACGCCTCCAGTCTCCATACAAGGATTTGAACCTTGGACCTCCTG
ATCCCAAATCAGGCGCTCTACCAAACTGAGCTACACGGAGTAAATTAAATTGGAGCGGATAATGAGAATC
GAACTCACATCATCAGATTGGAAGTCTGAGGTAATACCATTATACGATATCCGCAAATTTGGTGCGAGAA
GTGGGACTCGAACCCACAAGGAAATCATTCCGCAGCATTTTAAGTGCTGTGCCTTTACCAATTTGACCAT
TCTCGCGCTGGGAATAAAGGACTCGAACCTTTGCATCTAGCAGTCAAAGTGCTATGCCTTACCAACTTGG
CTAATTCCCAATTATTAACAAAGGCTCTCTAACAAGAACCCTTGATGATAGAGGGTATTAATCAGTGCGG
TATGAGTTAATAATAACAAATAATTCTTAAAGCATATTTACCATTTATGATGATACGTATTTACGATACA
TTCAAGACCCAAAGGATTCTTGAAAATATCATATTCAAGAGGACCTTTTTCTGTTTCAATAAAGAAATCA
AAATTTACTGTATTAAATTTACGGTCTTCCTTTACTAATTTAACTTGAGAAGATGAACGATCAATGTAAA
CCTTTTCAACTTCAAAACACGTTAAAATGCCATAATCATCAATCAAGGCTTTAGCTGCTTCTTGATCATA
TTTATATCCATTTTCAACGGATGATACTTTCGCATAAAGAATCATCATCAACCTCTATCAACAATAGCAT
GAGTATGGGCATTTACGATTTGCCACCAGTCGAAACGATTGGAACCATAATCTGGTTTATTTTCATTTTC
TTTAATGATATCACGCAGTTTATCTTCTGTTTCAGCATACGCAATTAAATCATCATATCCACCACAAGGA
TAATAATTATCACCTGCGAATAAAAGAAAATTTACCTTAGATGGATTTACGTAATAATGGTCTTTAGGAT
ATTTAGTTCCTCTCCAATCAGTTACTTCAACATAACGGTAAGAAAATCCATTTTTACTTTCAATCCAACT
CCACGCTTCAAAAGGAGTATTAAAAACTTTATCAGGTATTAAATTACCTTCAAAATGAGAAGGATTTGCA
TAATCCCCGGCATAAACATAATATTCGTTAATACTCATTTATTCACCTTTAGAAATTTTATCCATAACGA
TAGCAATTAAACCAATTAAAAATGCTACTACAAGTGAAAACACATTTTCTGCTGTAGTTAATAATCCGCA
TATAAATCCAACAAACATTGAAAAACTAAAAGCAGAAGCAGAAATTGCAATAGCAACATTTCGAATTAAT
TCACAGCGTTTCATTTTATTCTCCTCAGTAGTTGATAGGGTAATAGTATCACAGCTAAAACCCTATGTAA
ACAACTTTGTGAAATATTTATTACAAAAGATTTTTAGCAATAATCTTGAGATGTGCCGCAGAAATGTGTT
TAGCTTTAAACAACGCAGTTTCTTCAGCAGGAGAGATAACGATTGTAGCACCATCCTTTTTAGCAGACCA
CCCATCACCTAGGTAAACAGTACCTTTGATTTCTTCGCCATCAACCAGACTAATCATTGGTTTACCTTCT
CGTCCTTTATTTGCTTTAATAACTTCAGAAGTAAGAGTAGCTTCGGTAATGGTAGAAACCGGGGTAGTTG
TAGAAGTAAATTCTTTAAATGTTTTCATTTTTATTTTCCTAATTAATTTTGATGAGGTAATAGTATCACT
ACCTCATCAGTATGTAAACAACTTTGTGAAATTATTTTAAATCATCTGCCCAATCGAGTTTAAGAGGCTC
TTTGTATTCACGATCTAATACGACCGGAATTTGTACATCACCGCTAAATGATAAGGGCCCAACATTATAA
GACAATGTTATATGCGGTGTGTAATCATCAAAATCATGTGTAGCACCTAGTGCCCGCGCATACATGTGTC
GACAGCGCAGATATTCAGAATCTAGCACAAGTACAAGAGTCGATCCATCTTGTGTTTTCCATACTTCTAA
ATGTCCAGAAGAAGCTACTTCAAAACTTCCACTCGATGGAACATATGGAACATTTACTCTTGAATAACAT
ATAGTCGAATGAATTTTTTCTCTAGGAACTGGATTAGGAACACGTAAAGAGCGCTGAAGTTCTTCCAGCG
CATCAAGTGTTAATTCTGAAAACTTAGCTGCTACATAAAGACCCGTTGAAAAGTCTTTAAATTCCATCAT
TCTTCATCTTTTGCTTCATCTGCAGATTCAGCAGTAAGATTTTTGACAGCTTCAACGATTTCTTCAACTT
TGATAGTATCGCCAGTGATACCTACTGCACGAGCAATTTCAGCCAAAGTTCCTTGCAGAATTTTGGATTC
TTCCATCAGACGAGCAGCTTGATCCTGCGTATCAAGAATGCGAGATTTCAGAGTTACGATTTCAGCAGAC
AGTTTTTGTTCAACAGTTTGTTCAGACATTATAGTACCTTTAGTGTATTTTTAATTTTAGAAAAAAGTTC
TTCAAGAGAACCATCGTTTGTAATTACTAAATCGCCATCACGAATTGGCAATCCAGCTTCTGTAATATGT
GTATCATTGGATTTTTGACCAGGACGAACTACATGAATTACTGTAGCACCCATCGCCCTAGCCGCATCCA
TTTCATGATCTTGACGGGTATCAGGAACGATATAATAATCATAACCTGAGTTAAATTTATCAAGATAATC
TAAAGCAAATAATTTTACCCAGTACATGCGGTCGAAGTTATTAACAATCAAATCCGTACCTAGGGCTTGC
ATCAGACGACGGACTGACCATTGATCTTCAATATTATTTATAACGTCAGTAATCTTATTAAATGCTACGA
AATTAACTGATTCTTTTCCTTCGTCATCAAAAACAAACACACCETTAATTGGGCTTTTACCATTAAGATA
ACAAAATGCTTGTTCCATAATCGTGATTACTTCTAATTTAGTCAGATTTAAATTAGTCTCACGATCATAG
TCAATTCCTTCAAACTCTTTACGAGTTAAGCAAGGATAGTCAGTGTTTGCTGCAAATACTCCCCATGCAT
AAGCCAATGCATCCTTAATAGGACCAGCAAGTTGGTATTTAACTGCAGAATAATTGCTCATGATAAAATC
AGCAGTAGTATCTTTTCCACTACGCTTTACACCGCTTAAAAAGATTAGTTTCATGTGTTTCTCCTCAAAT
TTAATTAAGATTATAACACACAAAACTGAAGCATTAAACTTCTGCTATAATTTTACCATCTTTTTCTACT
TGAAAATAGGTGTAAGGAATTGTTGCAGTACATACTAAAGCCGGGTCTGAATCTTCCGTGTAGCTAAATT
CTACTTCAGATAGGTCAGAAACCCAAGGCTTATAAAAATTTATTGACATCACGATTTCAGTTTTGCTATT
ATCTAAGATGTAAAGCGTAATGTACTCAGGACCTGTTTTTTGGGCAGTATTTTCACCTGTAAGATAGTTG
CTAGTTCCTAGCATCCATTCATACATTCCTATCCACGACTTAAGTTCTTCATCAACTATAAATCTCACAA
TGAGTGGATCATACTCAAATGTAACACCTGGACGTTGTGCTCGGCCCAGTCCAAACGGCCCAGTCACGGT
ATCAGTAACAGGTATTCTAATTCCTGGAATAGGAACTGACTGAGCATTTAAAGTAAAAGCAGATGTAGTA
TTACTATGTGGTATTGATACTACAAAGTTAGTTGTATTTGCTTGGTTAAAAATTTGTTGCAGAGCTTGCG
ACATATATTCCTCATAATGCTTTATAACTGTTGGTGGTATAATGGGTCTAAGTCCCTTCCATTCAATTCC
ATTTAGAACAAACAACAGAAAAGAATGGAAGATAATAGAATTAGATATTTGACCAGACTTTGTTTGCAGA
GAAACGTTTTCCTTTTGAAACGAACTGCTGAAGTGGCATCAACACAACGTTCGCCCAGTCTTTCGGGGCG
ATTTCAACAAGGCTACCCATAATATTACCAGGTATATATGCCTTAATCATTTGGTCTGCACCCCTAAATC
CTTTCACTTGACTCCAATCAATTTTTAATTTCGTTTTATTAGTAATAGTAGGTGTATTTGCATATTGCTT
TAAAAGCTCTTCTAGGAATTGCTGACGAGCTTTAGGTGGAATATAGTGCAAGTTTAATCCGTACATTAAA
TTATGCTTACCTAAACCAAGGTAAATTATCAAAGGAAATTTATCCCAGTAAGGAAGAGTTTCCTTGTGTT
TAGCATCATAAGCAAAAGCATATATTCGTCCCGGCTGCGGGCGAACAACTTTATGTCCTTTTACTTGCTT
AATAGTTTCAGCAAACCACTTTCTGGTTTTATTATTAATTGCTGCGCCTTCATTACGAATTTTATCACGC
AATGTTTGTCTGAATGAATTTATCATAAGCAGTTGTCTTTCTTGCTTATTGAGTTTATTCATTGGTTTTG
ATTCAAGTTTTTGAATCTTTTCAGCCGTTTTAATTCCTGAAGCATATTTTGACATTGCTGAAGTAAACGT
AGAGTATTTGATTCCTCTTTCTTCAGCAAATTGCTTTCCTGTCATTCCTTTTGCTTTGGCCTTTTTATAT
TCAAGACCTATCTGAATCCATTTCTTTTCGTTTAATGATTGCTTAACCTTTGGAACTTGGGGAGTGCTTT
CATTAATTATTTGAAAAATAGCCATTATGCCCCCTTAAAGCCAAGAGCTCGTAATCCATCTTCTGTTAGA
ATTCTAAATTTTATTCCACGCTTTTCAGCTAAAGATTGTGCTGCTTTCCATTTGTCAGTGTTCACAGACC
AGGTATAAATTTCATTCATAAATCTTTTCTTCGCTGCGGTCGTTAGATGTGCTGGTTTAACTGGTGGTTG
TGTTTCTTTTTTAGGTTTTATTTCAATAAAAAATTCTTGTCCAGAAGAATCTTTCATCCAAATATCCATG
AAGTATCTACGTTTTTTCCCTTCTGCATTACAAAAATAAGGAATTACTGCTGTTTCACTACCCCATGCAA
TAATTTCTGGATTTTTATCTAACCATTCAAAAAAGAATTTTTCCCAATTTGATCTATACGTAATTTTTTT
AGGGTCACCTCTATACTTTGATATATTTTTAGGAACCCATTTTCCAGAATATGCCATTGGATTCTCCTTA
TAAATAGATAATATATTTATAAACAGGAGGGCCCATGCTCTTTACATTTTTTGATCCGATTGAATATGCG
GCCAAAACGGTGAATAAAAACGCGCCGACTATTCCTATGACAGATATTTTTAGAAACTATAAAGACTATT
TTAAACGCGCTCTTGCGGGATACCGCTTACGTACTTATTATATTAAAGGTTCACCACGCCCGGAAGAATT
AGCAAATGCTATATATGGAAATCCACAGCTGTATTGGGTTTTATTGATGTGTAATGATAATTATGACCCG
TATTATGGATGGATTACTTCGCAAGAAGCTGCTTATCAAGCATCTATACAAAAATACAAAAACGTAGGTG
GAGACCAAATAGTATATCATGTGAATGAGAACGGTGAAAAATTTTATAATTTAATATCATACGATGATAA
TCCATATGTTTGGTATGATAAAGGCGATAAAGCTAGAAAATATCCTCAATATGAAGGAGCGCTTGCTGCG
GTCGATACGTATGAAGCTGCTGTTCTTGAAAATGAAAAACTTCGTCAAATAAAAATAATAGCAAAATCAG
ACATCAATTCATTTATGAACGACCTTATACGTATAATGGAGAAATCTTATGGAAATGATAAGTAATAACC
TTAATTGGTTTGTCGGTGTTGTTGAAGATAGAATGGACCCATTAAAATTAGGTCGTGTTCGTGTTCGTGT
GGTTGGTCTGCATCCACCTCAAAGAGCACAAGGTGATGTAATGGGTATTCCAACTGAAAAATTACCATGG
ATGTCAGTTATTCAACCTATAACTTCTGCAGCAATGTCTGGAATTGGAGGTTCTGTTACTGGACCAGTAG
AAGGAACTAGAGTTTATGGTCATTTTTTAGACAAATGGAAAACTAATGGAATTGTCCTTGGCACGTATGG
TGGAATAGTTCGCGAAAAACCGAATAGACTTGAAGGATTTTCTGACCCAACTGGGCAGTATCCTAGACGT
TTAGGAAATGATACTAACGTACTAAACCAAGGTGGAGAAGTAGGATATGATTCGTCTTCTAACGTTATCC
AAGATAGTAACTTAGACACCGCAATAAATCCCGATGATAGACCGCTATCAGAGATTCCGACCGATGATAA
TCCAAATATGTCAATGGCTGAAATGCTTCGCCGTGATGAAGGATTAAGATTAAAAGTTTATTGGGATACC
GAAGGATATCCGACAATTGGTATTGGTCATCTTATCATGAAGCAGCCAGTTCGTGATATGGCTCAAATTA
ATAAAGTTTTATCAAAACAAGTTGGTCGTGAAATTACAGGAAATCCAGGTTCTATTACAATGGAAGAGGC
GACGACTTTATTTGAGCGTGATTTGGCTGATATGCAACGGGACATTAAATCACATTCTAAAGTAGGACCA
GTCTGGCAAGCTGTCAACCGTTCTCGTCAAATGGCGTTAGAAAATATGGCATTTCAGATGGGTGTTGGTG
GTGTAGCTAAATTTAACACAATGTTAACTGCTATGTTAGCAGGAGATTGGGAAAAAGCGTATAAAGCCGG
TCGTGATTCATTGTGGTATCAACAAACAAAAGGCCGTGCATCCCGTGTTACCATGATTATTCTTACGGGG
AATTTGGAATCATATGGTGTTGAAGTGAAAACCCCAGCTAGGTCTCTATCAGCAATGGCTGCTACTGTAG
CTAAATCTTCTGACCCTGCTGACCCTCCTATTCCAAATGACTCGAGAATTTTATTCAAAGAACCAGTTTC
TTCATATAAAGGTGAATATCCTTATGTGCATACAATGGAAACTGAAAGCGGACATATTCAGGAATTTGAT
GATACCCCTGGGCAAGAACGATATAGATTAGTTCATCCAACTGGAACTTATGAAGAAGTATCACCATCAG
GAAGAAGAACAAGAAAAACTGTTGATAATTTGTATGATATAACCAATGCTGATGGTAATTTTTTGGTAGC
CGGTGATAAAAAGACTAACGTCGGTGGTTCAGAAATTTATTATAACATGGATAATCGTTTACATCAAATC
GATGGAAGCAATACAATATTTGTACGTGGAGACGAAACGAAAACTGTTGAAGGTAATGGAACTATCCTAG
TTAAAGGTAATGTTACTATTATAGTTGAAGGTAATGCTGACATTACAGTTAAAGGAGATGCTACCACTTT
AGTTGAAGGAAATCAAACTAACACAGTAAATGGAAATCTTTCTTGGAAAGTTGCCGGGACAGTTGATTGG
GATGTCGGTGGTGATTGGACAGAAAAAATGGCATCTATGAGTTCTATTTCATCTGGTCAATACACAATTG
ATGGATCGAGGATTGACATTGGCTAATATACTTCCAATGAGCGCTGATTTAGGAGAATCCATGGAAGGTT
CTTCTATCGACGTCACCTTTACCGCTCAATTAGAAACAGGTGAAACGTTAGTATCTATAAATATAACTAG
TTACGAAGAAACTCCTGGGGTTTTAGTAGAAGAAAATCGCTTATATGGAACATATGAATCTGTATTTGGT
TTCGGAAATGACGCGTTGAAATATCGTTTAGGCGATGAATTTAAAACTGCTGCTTCATGGGAAGAACTTC
CTACTGATTCTGATACTCAGTTGTATTTGTGGAAAGCTCCTCAAAACCTCCAGAAGACATTCACTTACGA
AGTAACATTAATATATGACTACCAAGAACAAAGTGAATCTGGGGGTTCTGGCAGTAATTCTAGGTCATCT
TCTGATACTACTGAACCGACAGATCCTCCTGCTCCAGTAAGAAAAACTCTAGTTAAAAATTATACTAAAA
CTATAGTTGGAAATTGGAGTCGTTGGGCTAATAAACTGAGAAAATATGCCTATGCAAGACCATAAATATT
TTTATTTGTATTCAATAACTAATAAAACAACAGAAAAAATTTATGTAGGCGTCCACAAAACTTCAAATTT
GGATGATGGGTATATGGGTTCTGGCGTTGCCATTAAAAATGCCATTAAAAAATATGGCATAGATAATTTT
TATAAGCATATTATAAAATTCTTTGAATCTGAAAAAGCTATGTATGACGCAGAGGCAGAAATAGTCACAG
AGGAATTTGTTAAATCTAAGAAAACTTATAATATGAAACTAGGCGGTATCGGTGGCTTCCCAAAACATAA
CACAGCGGGTGCTAAAAATGGATTTTACGGTAAATCTCATTCGCGTGAAACTAGATTGAAAATTAGCATT
AAATCGTCTAGAAAAAGAGGGCCTAGAGGGCTAGAGGTAAAACTCTGAAGATGTGTGGCGCCAATAACCC
AAGGTATGGCAAAATAGCCCCTAATGCTAAATCTGTTATTATCAACGGCGTTTTATATAAAAGTATTAAA
ATCGCAGCTAAAGCTCTTAATATAAATTATAGTACCTTAAAGGGGCGAGTTAAAGCGGGGTATTATAAAT
GTCAGGATTAAGTTATGATAAGTGTGTTACTGCTGGCCATGAAGCGTGGCCTCCAACAGTTGTGAATGCT
ACACAAAGTAAAGTATTCACTGGAGGAATTGCTGTTCTCGTAGCAGGCGATCCAATTACAGAACATACAG
AAATTAAAAAGCCGTATGAAACACATGGCGGAGTGACACAACCTAGAACTTCTAAGGTATATGTCACTGG
AAAGAAAGCTGTTCAAATGGCTGATCCAATATCATGCGGTGATACTGTGGCTCAGGCATCATCTAAAGTA
TTCATTAAATAGGATTTAAAATGGCAAATACCCCTGTAAATTATCAATTAACAAGAACAGCAAATGCTAT
TCCCGAGATATTCGTCGGGGGTACATTTGCTGAAATAAAACAAAACCTCATTGAATGGCTTAATGGCCAA
AATGAATTTTTGGATTATGATTTTGAAGGCTCAAGATTAAACGTTCTGTGTGACCTTTTAGCTTATAATA
CATTATACATTCAGCAGTTTGGTAATGCTGCTGTGTATGAAAGCTTTATGCGTACTGCTAACTTACGAAG
TTCAGTTGTTCAAGCTGCACAAGATAACGGATATTTACCTACTTCAAAATCCGCTGCGCAGACCGAAATT
ATGTTAACATGCACTGACGCATTGAATAGGAATTACATTACTATTCCTCGCGGAACTCGCTTTTTAGCAT
ATGCAAAAGATACTTCTGTTAATCCATATAACTTCGTTTCTAGGGAAGACGTTATTGCTATTCGTGATAA
AAATAACCAATATTTTCCGCGTTTAAAATTGGCCCAGGGACGTATAGTAAGAACTGAAATCATTTATGAT
AAATTAACACCTATTATCATTTATGATAAAAATATTGATAGAAACCAGGTTAAATTATACGTTGATGGAG
CGGAATGGATTAACTGGACGAGAAAGTCAATGGTTCATGCTGGTTCAACATCAACGATTTACTATATGCG
TGAAACTATTGATGGAAACACTGAATTCTATTTTGGTGAAGGTGAAATTTCTGTTAATGCTTCTGAAGGA
GCTTTGACCGCTAATTATATCGGAGGTCTTAAACCTACTCAGAACTCTACGATTGTTATTGAGTACATTA
GTACTAATGGTGCTGACGCGAACGGAGCAGTCGGATTTTCATACGCAGATACATTAACAAATATAACTGT
CATCAATATTAATGAAAATCCAAACGATGATCCAGATTTTGTTGGGGCAGATGGAGGCGGTGATCCAGAA
GATATTGAGCGTATTCGCGAATTGGGTACTATTAAACGCGAAACCCAACAACGATGCGTAACTGCGACTG
ACTATGATACATTCGTTTCAGAGAGATTTGGTTCTATTATTCAAGCTGTTCAGACTTTCACTGATTCTAC
TAAACCTGGGTATGCATTTATTGCTGCTAAACCTAAATCAGGATTGTATTTAACTACCGTACAGCGTGAA
GATATTAAAAATTATCTCAAAGACTATAATTTAGCTCCTATTACGCCATCAATTATTTCTCCTAATTATC
TTTTTATTAAGACTAATTTAAAAGTCACATATGCTTTAAATAAACTGCAAGAATCCGAACAGTGGCTTGA
AGGTCAAATAATTGATAAAATAGATCGCTATTATACCGAAGATGTAGAAATTTTTAACTCGTCTTTCGCT
AAATCTAAGATGTTGACATATGTAGATGATGCAGATCATTCTGTCATTGGTTCATCAGCGACTATTCAAA
TGGTTCGTGAAGTACAAAACTTCTATAAAACGCCTGAAGCGGGTATTAAATACAATAATCAAATAAAAGA
TCGTTCTATGGAATCTAATACGTTTTCATTTAATTCTGGACGAAAGGTTGTAAATCCTGATACTGGTTTA
GAAGAAGATGTATTATATGACGTTCGTATAGTATCAACAGACCGAGATTCTAAAGGAATTGGTAAAGTTA
TTATTGGTCCATTTGCTTCTGGCGATGTTACAGAAAATGAAAACATTCAGCCGTATACAGGCAACGATTT
TAACAAATTAGCAAATTCTGATGGACGCGACAAATACTATGTTATCGGTGAAATAAATTATCCAGCTGAT
GTGATTTATTGGAATATCGCTAAAATTAATTTAACATCTGAAAAATTTGAAGTTCAGACCATTGAATTAT
ATTCTGACCCAACCGATGATGTTATCTTTACTCGCGATGGTTCACTGATTGTATTTGAAAATGACTTACG
TCCACAATACTTAACTATCGATTTGGAGCCTATATCACAATGACAGTAAAAGCACCTTCAGTCACTAGTC
TCAGAATTTCCAAGTTATCCGCAAATCAGGTGCAAGTACGCTGGGATGACGTTGGTGCTAATTTCTACTA
TTTTGTAGAAATCGCTGAGACAAAAACAAACTCGGGGGAAAATCTCCCGAGTAATCAATATCGTTGGATT
AATTTAGGATATACAGCAAATAATAGTTTCTTTTTTGATGATGCTGATCCATTAACAACATACATTATTA
GAGTAGCCACAGCTGCGCAAGATTTTGAGCAGTCTGATTGGATTTATACCGAAGAGTTTGAAACTTTTGC
TACAAATGCTTATACATTTCAAAACATGATTGAAATGCAATTAGCCAATAAATTCATTCAGGAAAAATTT
ACTCTTAATAATTCTGATTATGTTAATTTTAATAATGATACTATAATGGCTGCATTGATGAATGAATCAT
TCCAATTCAGCCCATCGTATGTTGATGTTTCATCAATAAGTAATTTTATTATTGGTGAAAATGAGTATCA
TGAAATACAAGGTTCTATTCAGCAAGTATGTAAGGATATTAACCGAGTTTATTTGATGGAATCAGAAGGA
ATTCTATATCTTTTTGAGCGCTATCAACCTGTAGTTAAAGTATCCAATGATAAAGGACAAACCTGGAAAG
CTGTAAAGCTCTTCAATGACCGTGTAGGATATCCTTTATCTAAGACAGTATATTACCAATCTGCGAACAC
AACATACGTTCTAGGATACGACAAGATTTTCTATGGCCGCAAATCTACTGATGTTAGATGGTCAGCCGAT
GATGTCAGATTTAGTTCTCAGGATATAACATTTGCTAAACTTGGCGACCAATTACATCTAGGATTTGATG
TAGAAATTTTTGCCACTTACGCGACTTTACCAGCGAATGTATACCGCATTGCAGAAGCTATTACTTGCAC
CGATGATTACATTTACGTTGTCGCCAGAGACAAAGTTAGATACATAAAAACGAGTAATGCACTTATAGAT
TTTGATCCATTATCTCCAACATATTCGGAAAGACTTTTTGAACCTGATACCATGACTATAACCGGAAATC
CTAAAGCAGTATGCTATAAAATGGATTCTATCTGTGATAAAGTTTTTGCTCTTATTATTGGTGAAGTTGA
AACATTAAATGCTAATCCTAGAACATCAAAAATAATTGATTCCGCTGATAAAGGAATATATGTTTTAAAT
CATGACGAAAAAACATGGAAAAGAGTTTTTGGTAATACCGAAGAAGAAAGAAGACGTATTCAACCCGGAT
ATGCGAATATGTCAACTGACGGTAAATTAGTTTCTCTGTCTTCGAGTAATTTTAAATTTTTAAGTGATAA
TGTTGTTAATGACCCTGAAACTGCAGCAAAATATCAGTTAATTGGCGCTGTTAAATATGAATTTCCTCGT
GAATGGTTAGCTGATAAGCATTATCATATGATGGCATTTATAGCGGATGAAACATCTGATTGGGAGACTT
TTACTCCTCAACCAATGAAATACTACGCAGAACCATTCTTTAACTGGTCTAAAAAATCTAACACACGTTG
TTGGATAAACAACTCTGATAGAGCTGTGGTAGTTTATGCTGATTTAAAATACACTAAAGTTATAGAAAAT
ATTCCGGAAACATCACCAGATAGATTAGTTCATGAATACTGGGATGATGGTGATTGCACTATAGTAATGC
CAAATGTCAAATTCACTGGATTTAAAAAATACGCATCAGGAATGCTTTTCTATAAAGCCTCCGGTGAAAT
AATTTCTTACTATGATTTTAACTATCGTGTGAGAGATACAGTAGAAATTATTTGGAAGCCAACTGAAGTA
TTTTTAAAAGCATTTTTACAAAACCAAGAGCATGAGACTCCTTGGTCACCAGAAGAAGAGCGTGGATTAG
CTGACCCTGATTTAAGACCATTAATTGGCACAATGATGCCTGATTCTTATTTGTTACAGGATTCGAATTT
TGAGGCATTTTGCGAAGCATATATTCAGTATCTTTCTGATGGATATGGAACTCAATACAATAATTTACGA
AATTTAATTCGTAACCAATATCCACGAGAAGAGCACGCATGGGAATATTTGTGGTCAGAGATATATAAAA
GAAACATTTATTTAAATGCTGATAAACGCGATGCTGTTGCGAGATTCTTTGAATCACGTAGCTATGATTT
TTATTCTACTAAAGGAATTGAAGCATCATACAAGTTTCTTTTTAAAGTTCTTTATAATGAAGAAGTTGAA
ATTGAAATTGAATCTGGGGCTGGTACTGAATATGATATAATCGTTCAATCTGATTCTTTGACTGAAGATT
TAGTAGGACAAACGATTTATACGGCAACAGGAAGATGTAATGTTACTTATATAGAAAGAAGCTATTCTAA
TGGTAAATTGCAATGGACCGTAACTATTCATAATCTTTTGGGACGATTAATTGCTGGTCAAGAAGTTAAA
GCAGAAAGACTCCCTAGTTTTGAAGGCGAAATTATTCGTGGGGTTAAAGGAAAGGATTTGCTTCAAAACA
ATATAGACTATATTAATAGAAGTAGATCATACTATGTAATGAAAATTAAATCCAATTTACCTTCTTCCCG
CTGGAAATCTGACGTTATTCGTTTTGTTCATCCAGTAGGATTTGGATTTATAGCAATTACCCTTTTAACA
ATGTTTATTAATGTTGGTTTAACTCTTAAACATACAGAGACTATAATTAATAAATACAAAAACTATAAAT
GGGATTCTGGATTGCCTACTGAATATGCCGACAGAATAGCTAAATTAACTCCAACCGGTGAAATTGAGCA
TGATTCAGTAACAGGCGAAGCAATTTATGAGCCTGGCCCAATGGCTGGTGTAAAATATCCTCTTCCTGAT
GACTATAATGCTGAAAATAATAATTCAATATTTCAAGGTCAATTGCCGTCTGAACGACGTAAATTAATGA
GTCCTTTATTTGATGCATCTGGAACAACATTTGCGCAATTTAGAGATTTAGTTAATAAACGTCTAAAAGA
TAATATAGGAAATCCAAGAGACCCTGAAAATCCAACACAGGTTAAAATAGATGAATGATTCAAGTGTTAT
CTATCGTGCGATAGTTACTTCAAAATTTAGAACAGAAAAAATGTTGAATTTTTATAATTCAATTGGAAGT
GGTCCGGATAAAAACACTATCTTTATCACATTTGGAAGATCAGAACCGTGGTCATCAAATGAAAATGAGG
TGGGCTTTGCCCCACCTTATCCAACCGATTCTGTATTAGGCGTAACTGACATGTGGACGCATATGATGGG
AACAGTAAAAGTTCTTCCATCAATGCTTGATGCTGTTATTCCTCGCAGAGATTGGGGAGATACTAGATAT
CCGGATCCATACACATTTAGAATTAACGATATTGTAGTGTGTAACTCAGCTCCTTACAACGCTACTGAAT
CAGGCGCTGGTTGGTTAGTGTATCGTTGTTTAGATGTTCCTGATACCGGAATGTGTTCAATAGCATCTTT
AACTGATAAAGATGAATGCCTTAAGTTAGGTGGAAAATGGACTCCTTCTGCTAGGTCAATGACTCCGCCT
GAAGGTCGAGGAGATGCTGAAGGAACAATTGAACCCGGAGACGGGTATGTGTGGGAATATCTATTTGAGA
TTCCGCCTGATGTATCTATAAATAGATGCACGAATGAATATATCGTGGTTCCTTGGCCTGAGGAATTAAA
AGAAGACCCGACTAGATGGGGATATGAAGATAATCTCACTTGGCAACAAGATGATTTTGGATTAATTTAC
CGTGTTAAGGCAAATACTATCCGTTTTAAAGCATATTTAGATTCAGTTTATTTTCCTGAAGCTGCATTAC
CAGGAAATAAAGGATTTAGACAAATATCAATAATCACGAATCCTCTTGAAGCTAAAGCTCATCCAAATGA
CCCAAACGTTAAAGCTGAAAAGGATTATTATGACCCAGAAGATTTAATGAGGCATTCGGGTGAAATGATT
TATATGGAAAATAGGCCACCTATTATTATGGCAATGGATCAAACAGAAGAAATCAATATTCTGTTTACAT
TTTAAATTAAGGGAGCCCATGGGCTCCCTTTTTCTTTATAAATACTATAAACTCATAAGGAAACCGCTAT
GTTCATTCAAGAACCAAAGAAATTGATTGATACCGGCGAAATTGGTAACGCTTCTACTGGTGATATCTTA
TTCGACGGTGGTAATAAAATTAATAGTGATTTTAACGCAATTTATAATGCGTTTGGCGATCAGCGTAAAA
TGGCAGTAGCAAATGGCACTGGAGCAGATGGTCAAATTATCCATGCTACTGGATATTATCAAAAACACTC
TATTACAGAGTACGCAACTCCAGTGAAAGTTGGCACTAGACATGATATTGATACCTCTACTGTAGGTGTT
AAAGTTATCATTGAAAGAGGCGAACTCGGCGATTGTGTTGAATTCATTAACTCTAATGGATCAATATCAG
TTACTAATCCTTTGACAATTCAAGCTATTGATTCAATTAAAGGTGTTTCAGGTAATTTAGTAGTAACTAG
CCCATATAGTAAAGTTACTTTACGCTGTATTTCATCTGATAATTCTACGTCGGTTTGGAATTATTCTATT
GAAAGTATGTTTGGACAAAAGGAATCACCAGCTGAAGGTACATGGAATATTTCTACATCTGGATCAGTTG
ACATTCCATTATTTCATCGTACTGAATACAATATGGCTAAATTGCTAGTTACGTGCCAATCGGTAGATGG
AAGAAAAATTAAAACAGCAGAAATAAATATTCTTGTGGATACTGTTAATTCAGAGGTAATTTCTTCTGAA
TATGCTGTCATGCGAGTTGGGAATGAAACCGAAGAAGACGAAATCGCTAATATTGCATTTAGTATTAAAG
AAAATTATGTAACGGCGACTATAAGTTCTTCAACTGTCGGTATGAGAGCAGCAGTTAAAGTTATCGCTAC
GCAGAAAATCGGGGTGGCTCAATAATGAAACAAAATATTAATATCGGTAATGTTGTAGATGATGGTACCG
GTGACTACCTGCGTAAAGGTGGTATAAAAATAAATGAAAACTTTGATGAGCTTTATTATGAACTCGGTGA
TGGTGATGTTCCATATTCAGCCGGTGCCTGGAAAACTTATAATGCTTCATCAGGACAAACATTAACAGCA
GAATGGGGAAAATCATACGCTATTAATACATCTTCTGGAAGAGTGACTATAAATCTTCCAAAGGGTACAG
TTAATGATTACAACAAGGTAATTAGAGCTAGAGACGTATTTGCTACATGGAACGTCAACCCAGTTACACT
AGTAGCTGCTTCCGGCGATACGATTAAAGGGTCTGCAGTACCAGTTGAAATTAATGTTCGATTCAGCGAT
TTAGAACTAGTGTATTGTGCCCCAGGACGTTGGGAATATGTCAAAAATAAACAAATTGACAAAATTACCA
GTTCAGACATTAGTAATGTAGCTCGCAAAGAATTTTTAGTTGAAGTTCAAGGACAAACAGACTTTTTAGA
TGTTTTCCGTGGAACTAGTTATAATGTAAATAACATCAGAGTAAAACATCGTGGTAACGAATTGTATTAC
GGCGATGTGTTTAGCGAAAACAGCGATTTTGGCTCTCCAGGCGAAAATGAAGGAGAACTGGTTCCTCTTG
ATGGATTTAACATTCGATTAAGACAGCCTTGTAATATTGGTGACACTGTTCAAATTGAAACATTTATGGA
TGGTGTATCACAGTGGAGAAGTTCATATACAAGACGTCAAATTAGATTGTTAGATTCAAAATTAACGTCA
AAAACTTCTTTAGAAGGAAGCATTTACGTTACTGATTTATCAACAATGAAATCAATTCCATTTTCTGCTT
TTGGATTAATTCCAGGAGAACCTATTAATCCTAACTCTCTTGAGGTTCGTTTTAACGGGATTTTACAGGA
ATTGGCTGGCACAGTTGGAATGCCATTATTTCATTGTGTTGGTGCCGATTCAGACGATGAAGTAGAATGC
TCTGTTTTAGGTGGAACTTGGGAACAATCTCATACCGATTATTCAGTTGAAACTGATGAAAACGGCATAC
CAGAAATTTTACATTTCGATAGCGTATTTGAGCATGGTGACATTATCAATATCACCTGGTTTAATAATGA
TTTGGGTACATTATTAACAAAAGATGAGATTATTGATGAAACTGATAATCTCTATGTATCGCAAGGACCT
GGAGTAGATATTTCTGGTGATGTAAATTTAACAGACTTCGATAAAATTGGTTGGCCAAATGTAGAAGCAG
TTCAATCTTATCAACGCGCATTTAATGCTGTTTCAAATATCTTTGATACGATTTATCCTATTGGAACTAT
ATATGAAAACGCTGTTAATCCAAATAACCCTGTTACATATATGGGATTCGGCTCATGGAAATTATTTGGG
CAAGGAAAAGTTTTAGTTGGATGGAATGAAGATATTTCGGACCCTAACTTTGCTCTAAATAACAACGATT
TAGATTCGGGTGGAAATCCTTCACATACCGCAGGTGGAACAGGTGGTTCTACTTCTGTTACATTGGAAAA
TGCTAATCTTCCTGCAACTGAAACAGATGAAGAAGTTCTAATAGTTGATGAAAATGGATCAGTCATTGTT
GGTGGGTGTCAATACGATCCAGATGAATCCGGTCCAATTTACACTAAATACCGTGAAGCTAAAGCATCTA
CTAACTCTACTCACACTCCGCCAACATCAATAACTAACATTCAACCATATATTACAGTTTATCGTTGGAT
AAGGATTGCATAATGAGTTTACTTAATAATAAAGCGGGAGTTATTTCCCGCTTAGCCGATTTTCTTGGTT
TTAGACCTAAAACTGGCGACATTGATGTAATGAATCGTCAATCAGTCGGGTCAGTGACAATATCTCAATT
AGCGAAAGGATTTTATGAACCAAACATAGAATCAGCTATTAATGACGTTCATAATTTTTCTATAAAAGAC
GTTGGCACAATTATTACTAATAAAACTGGTGTTTCTCCTGAGGGTGTTTCTCAAACTGATTATTGGGCAT
TTTCTGGAACTGTAACAGACGATTCTCTTCCTCCGGGTTCTCCTATTACGGTATTAGTATTTGGTCTTCC
AGTTTCAGCAACAACTGGAATGACGGCAATTGAGTTTGTTGCAAAAGTTCGCGTTGCACTACAAGAAGCT
ATTGCGTCATTTACTGCTATCAATTCATATAAAGACCATCCAACTGATGGTAGTAAATTAGAAGTTACTT
ATTTAGATAATCAAAAACATGTATTAAGCACATATTCTACATATGGAATAACTATTTCCCAAGAAATTAT
ATCTGAGTCTAAGCCTGGCTATGGTACATGGAATTTATTGGGCGCACAAACTGTAACTTTAGATAATCAG
CAGACTCCTACAGTATTTTATCATTTTGAGAGAACAGCATGAGTAATAATACATATCAACACGTTTCTAA
TGAATCTCGTTATGTAAAATTTGATCCTACCGATACGAATTTTCCACCGGAGATTACTGATGTTCACGCT
GCTATAGCAGCCATTTCTCCTGCTGGAGTAAATGGAGTTCCTGATGCATCGTCAACAACAAAGGGAATTC
TATTTATTCCCACTGAACAGGAAGTTATAGATGGAACTAATAATACCAAAGCAGTTACACCAGCAACGTT
GGCAACAAGATTATCTTATCCAAATGCAACTGAAACTGTTTACGGATTAACAAGATATTCAACCAATGAT
GAAGCCATTGCCGGAGTTAATAATGAATCTTCTATAACTCCAGCTAAATTTACTGTCGCCCTTAATAATG
CGTTTGAAACGCGAGTTTCAACTGAATCCTCAAATGGTGTTATTAAAATTTCATCTCTACCGCAAGCATT
AGCTGGTGCAGATGATACTACTGCAATGACTCCATTAAAAACACAGCAGTTAGCTATTAAATTAATTGCG
CAAATTGCTCCTTCTGAAACCACAGCTACCGAATCGGACCAAGGTGTTGTTCAATTAGCAACAGTAGCGC
AGGTTCGTCAGGGAACTTTAAGAGAAGGCTATGCAATTTCTCCTTATACGTTTATGAATTCATCTTCTAC
TGAAGAATATAAAGGCGTAATTAAATTAGGAACACAATCAGAAGTTAACTCGAATAATGCTTCTGTTGCG
GTTACTGGCGCAACTCTTAATGGTCGTGGTTCTACGACGTCAATGAGAGGCGTAGTTAAATTAACTACAA
CCGCCGGTTCACAGAGTGGAGGCGATGCTTCATCAGCCTTAGCTTGGAATGCTGACGTTATCCAGCAAAG
AGGTGGTCAAATTATCTATGGAACACTCCGCATTGAAGACACATTTACAATAGCTAATGGTGGAGCAAAT
ATTACGGGTACCGTCAGAATGACTGGCGGTTATATTCAAGGTAACCGCATCGTAACACAAAATGAAATTG
ATAGAACTATTCCTGTCGGAGCTATTATGATGTGGGCCGCTGATAGTCTTCCTAGTGATGCTTGGCGCTT
CTGCCATGGTGGAACTGTTTCAGCGTCAGATTGTCCATTATATGCTTCTAGAATTGGAACAAGATATGGC
GGAAACCCATCAAATCCTGGATTGCCTGACATGCGTGGTCTTTTTGTTCGTGGTTCTGGTCGTGGTTCTC
ACTTAACAAATCCAAATGTTAATGGTAATGACCAATTTGGTAAACCTAGATTAGGTGTAGGTTGTACCGG
TGGATATGTTGGTGAAGTACAGATACAACAGATGTCTTATCATAAACATGCTGGTGGATTTGGTGAGCAT
GATGATCTGGGGGCATTCGGTAATACCCGTAGATCAAATTTTGTTGGTACACGTAAAGGACTTGACTGGG
ATAACCGTTCATACTTCACCAATGACGGATATGAAATTGACCCAGAATCACAACGAAATTCCAAATATAC
ATTAAATCGTCCTGAATTAATTGGAAATGAAACACGTCCATGGAACATTTCTTTAAACTACATAATTAAG
GTAAAAGAATGACAGATATTGTACTGAATGACTTACCATTCGTTGACGGCCCTCCTGCAGAGGGCCAGAG
CCGCATTTCCTGGATTAAAAACGGCGAAGAAATATTAGGAGCTGACACACAGTATGGAAGTGAAGGCTCA
ATGAATAGACCTACGGTTTCTGTACTAAGAAATGTTGAAGTTCTTGATAAAAACATTGGAATACTTAAAA
CATCTTTAGAAACCGCAAATAGTGATATTAAAACAATTCAGGGCATCTTAGATGTATCTGGTGATATTGA
AGCTTTGGCCCAAATAGGTATCAATAAAAAGGATATTTCTGACCTCAAAACGCTAACCAGTGAACACACA
GAAATATTAAATGGAACTAATAATACGGTTGACAGTATTCTTGCCGATATTGGTCCATTTAACGCCGAGG
CCAACTCTGTATACAGAACGATCAGAAATGATTTACTGTGGATAAAGCGTGAACTTGGACAATACACTGG
TCAAGATATTAATGGTCTTCCTGTTGTAGGAAATCCTAGTAGTGGAATGAAGCATCGCATTATTAATAAT
ACTGATGTCATCACTTCGCAGGGAATACGTTTAAGCGAATTAGAAACAAAATTTATTGAATCTGATGTAG
GTTCTTTGACCATTGAAGTTGGTAATCTTCGTGAAGAGCTTGGACCGAAACCACCATCATTTTCACAGAA
CGTTTATAGTCGTTTAAATGAAATTGACACTAAACAGACAACAGTTGAGTCTGACATTAGTGCTATTAAG
ACCTCAATAGGATATCCAGGAAATAATTCGATTATCACGAGTGTTAATACAAACACTGATAATATTGCAT
CTATTAATTTAGAGCTAAATCAAAGTGGAGGTATTAAACAGCGTTTAACCGTTATTGAAACTTCCATTGG
TTCAGATGATATTCCTTCGAGTATTAAAGGTCAAATCAAAGATAATACAACTTCAATCGAATCTCTAAAT
GGAATCGTCGGTGAAAACACTTCATCTGGCTTAAGAGCGAATGTTTCATGGTTAAACCAAATTGTTGGAA
CTGATTCTAGCGGTGGACAACCTTCTCCTCCTGGGTCTCTTTTAAACCGAGTTTCTACAATTGAAACTTC
TGTTTCAGGCTTGAATAACGCTGTTCAAAACCTACAAGTAGAGATTGGTAATAACAGCGCAGGAATTAAA
GGGCAAGTTGTAGCGTTAAATACTTTAGTAAATGGAACTAATCCAAACGGTTCAACTGTTGAAGAGCGCG
GATTAACCAATTCAATAAAAGCTAACGAAACTAACATTGCATCAGTTACACAAGAAGTGAATACAGCTAA
AGGCAATATATCTTCTTTACAAGGTGATGTTCAAGCTCTCCAAGAAGCCGGTTATATTCCTGAAGCTCCA
AGAGATGGGCAAGCTTACGTTCGTAAAGATGGCGAATGGGTATTCCTTTCTACCTTTTTATCACCAGCAT
AACATGGGGCCGCAAGGCCCCAAAGGATTTTAAATGTCAGGATATAATCCTCAGAATCCAAAGGAACTCA
AAGATGTCATTCTAAGACGTTTAGGGGCTCCAATTATTAATGTTGAGTTAACACCCGATCAAATTTACGA
TTGTATCCAGCGTGCCCTAGAATTATACGGTGAATACCATTTTGATGGACTCAATAAAGGTTTTCATGTT
TTTTATGTAGGGGATGATGAAGAAAGGTACAAGACCGGAGTCTTCGATTTAAGAGGTTCTAACGTATTTG
CAGTAACCCGCATTTTACGCACAAATATTGGGTCAATAACATCTATGGATGGAAACGCTACATATCCGTG
GTTTACTGACTTTCTTTTAGGAATGGCTGGTATTAATGGCGGAATGGGAACGTCTTGTAATAGATTTTAT
GGACCAAATGCCTTTGGAGCTGATTTAGGATATTTTACCCAGCTTACCAGTTATATGGGAATGATGCAAG
ATATGCTCTCTCCTATTCCAGACTTTTGGTTTAATTCAGCAAATGAACAGCTCAAAGTCATGGGAAACTT
CCAAAAATATGATTTAATTATCGTAGAAAGCTGGACTAAATCATACATTGATACAAACAAAATGGTTGGA
AATACAGTAGGATATGGAACAGTCGGTCCACAAGATAGCTGGTCATTATCTGAACGATATAATAACCCAG
ACCACAATTTAGTAGGTCGTGTTGTCGGCCAAGATCCGAATGTTAAACAGGGTGCTTATAATAATCGTTG
GGTGAAAGACTATGCAACAGCTTTAGCTAAAGAATTGAACGGTCAAATTTTAGCACGCCACCAAGGTATG
ATGCTTCCGGGCGGTGTTACAATTGATGGGCAGCGCTTAATAGAAGAAGCCAGATTAGAAAAAGAAGCAC
TGCGCGAAGAATTATACTTACTTGATCCTCCATTTGGAATTTTGGTAGGTTAATATGGCTACTTATGATA
AAAATCTTTTTGCTAAATTGGAAAACCGCACAGGTTATTCTCAGACCAATGAAACTGAAATATTAAATCC
TTATGTAAATTTCAATCATTATAAAAACAGCCAAATATTAGCTGATGTATTAGTAGCTGAAAGCATTCAA
ATGCGAGGTGTAGAATGCTATTATGTTCCAAGAGAGTATGTTTCCCCTGATTTGATATTCGGCGAAGACT
TAAAAAATAAATTTACTAAAGCTTGGAAATTTGCTGCATATTTAAATTCATTTGAAGGATATGAAGGAGC
TAAATCGTTCTTTAGTAACTTTGGTATGCAAGTACAAGACGAAGTGACTTTATCTATTAACCCAAATTTA
TTTAAGCATCAAGTTAACGGAAAAGAACCCAAGGAAGGTGATTTGATATATTTTCCTATGGATAACAGCT
TATTTGAAATTAACTGGGTTGAACCATATGATCCATTTTATCAATTAGGCCAAAACGCTATTCGTAAAAT
TACGGCAGGTAAATTCATTTATTCTGGAGAAGAAATTAATCCAGTTCTACAGAAAAATGAAGGAATTAAC
ATTCCAGAATTTAGTGAATTAGAATTAAATGCTGTTCGCAATCTTAACGGTATTCATGACATTAATATTG
ATCAGTATGCTGAAGTAGATCAAATTAATTCTGAAGCTAAAGAATACGTTGAACCTTATGTTGTTGTCAA
TAACAGAGGCAAATCTTTCGAATCTAGCCCATTTGACAATGATTTCATGGATTAATAAATATTATAAACT
AATTAAAGCCCGGATTAGGAGAAGTCATGTTTGGTTATTTTTATAATTCGTCTTTTAGACGATATGCTAC
CTTGATGGGCGATTTGTTTTCAAATATCCAAATCAAACGTCAGTTAGAATCTGGTGATAAGTTTATACGT
GTTCCTATTACGTATGCATCAAAGGAACACTTTATGATGAAATTGAATAAATGGACATCAATAAATTCAC
AAGAAGATGTAGCTAAAGTTGAAACTATTCTACCTCGTATAAATTTACATTTAGTTGATTTTAGCTATAA
TGCTCCATTTAAAACAAACATTTTAAATCAGAATTTACTGCAAAAAGGTGCAACTTCTGTAGTATCGCAG
TATAATCCATCTCCTATTAAAATGATTTATGAATTGAGTATCTTTACTCGCTATGAAGATGATATGTTTC
AAATAGTTGAACAGATTCTTCCATATTTTCAACCTCATTTTAATACAACTATGTACGAGCAGTTTGGAAA
TGATATTCCATTTAAAAGGGATATTAAAATTGTACTGATGTCTGCTGCTATAGACGAAGCTATAGATGGG
GATAATTTATCTCGTCGTAGAATTGAATGGTCATTAACATTTGAAGTAAATGGATGGATGTATCCTCCAG
TAGATGATGCAGAAGGATTAATTCGTACTACTTATACAGATTTTCACGCCAATACAAGAGATTTGCCTGA
CGGCGAAGGTGTTTTTGAATCTGTCGATAGCGAAGTTGTTCCTCGAGATATTGACCCAGAAGACTGGGAT
GGAACAGTAAAACAAACTTTCACTAGTAATGTAAATAGACCAACACCGCCAGAACCTCCTGGCCCAAGAA
CATAGAGGTTATTATGGAAGGTCTTGATATAAACAAACTTTTAGATATTTCTGACCTCCCCGGAATTGAC
GGGGAGGAAATCAAAGTGTATGAACCTCTGCAATTAGTAGAAGTTAAAAGCAATCCACAAAACCGTACTC
CAGACTTAGAAGATGATTATGGAGTAGTTCGTCGAAATATGCATTTTCAGCAACAAATGCTAATGGACGC
TGCCAAGATTTTTCTTGAGACAGCAAAGAATGCTGATTCTCCTCGTCACATGGAAGTATTTGCAACTCTT
ATGGGGCAAATGACTACGACGAACAGAGAAATACTGAAGCTTCATAAAGATATGAAAGACATTACATCTG
AGCAGGTTGGCACCAAAGGCGCTGTTCCTACAGGTCAAATGAATATTCAGAATGCGACAGTATTCATGGG
TTCACCAACAGAATTAATGGACGAAATTGGTGATGCTTACGAGGCTCAAGAAGCTCGTGAGAAGGTGATA
AATGGAACAACCGATTAATGTATTAAATGATTTCCATCCGTTAAATGAAGCTGGAAAAATTTTAATAAAA
CACCCAAGCTTAGCGGAAAGAAAAGATGAAGATGGAATTCATTGGATAAAATCTCAGTGGGATGGAAAAT
GGTATCCTGAAAAATTCAGTGATTACCTTCGTCTACACAAAATAGTAAAAATTCCAAACAACTCTGATAA
GCCTGAATTATTTCAAACTTATAAAGATAAGAATAATAAAAGATCTCGGTATATGGGTCTTCCTAACTTG
AAACGAGCTAATATTAAAACACAATGGACTCGTGAAATGGTTGAGGAATGGAAAAAATGCCGAGATGATA
TTGTTTATTTTGCAGAAACATACTGTGCCATTACTCATATTGACTATGGTGTCATAAAGGTTCAATTACG
TGACTATCAGCGTGATATGCTCAAAATAATGTCATCTAAACGTATGACTGTTTGTAATCTATCGCGCCAG
CTCGGTAAAACCACCGTAGTAGCTATTTTCCTTGCACACTTTGTATGTTTTAACAAAGATAAAGCTGTAG
GTATTCTTGCACACAAAGGCTCAATGTCTGCGGAAGTTTTAGACCGTACTAAGCAAGCAATTGAACTGCT
TCCTGACTTTTTACAACCAGGAATTGTTGAATGGAATAAGGGTTCAATTGAACTAGATAATGGTTCTTCA
ATTGGCGCTTATGCTTCCTCTCCTGACGCAGTTCGTGGTAACTCGTTCGCAATGATTTACATTGACGAAT
GTGCGTTTATTCCAAACTTCCATGATTCCTGGCTTGCTATTCAACCAGTAATTTCATCTGGTCGTCGTTC
GAAAATTATTATTACTACGACTCCTAATGGATTAAATCATTTTTATGATATTTGGACTGCTGCTGTCGAA
GGTAAATCTGGATTTGAACCATATACTGCTATTTGGAATTCAGTTAAAGAACGTCTTTATAACGATGAAG
ATATTTTTGACGATGGATGGCAATGGAGCATACAAACCATTAATGGTTCTTCATTAGCTCAATTCCGTCA
AGAACATACTGCAGCGTTTGAAGGGACTTCTGGTACATTAATTTCAGGAATGAAATTAGCTGTTATGGAT
TTTATTGAAGTAACACCAGATGATCATGGTTTTCACCAATTTAAAAAACCTGAACCAGATAGAAAATATA
TTGCAACTCTAGATTGCTCAGAAGGTCGTGGGCAAGATTACCACGCTTTGCATATTATTGATGTTACTGA
TGATGTGTGGGAACAGGTTGGTGTTTTGCATTCAAACACTATTTCTCATTTAATTCTACCTGACATCGTT
ATGCGTTATTTAGTAGAATACAATGAATGCCCAGTTTATATTGAATTAAATAGTACTGGTGTGTCAGTTG
CAAAATCGCTTTATATGGATTTAGAATACGAAGGTGTTATCTGCGATTCATATACTGATTTAGGAATGAA
ACAAACTAAACGCACGAAAGCAGTAGGATGTTCCACGCTAAAAGACCTTATTGAAAAAGATAAGCTTATT
ATTCATCACCGAGCGACTATTCAAGAATTTAGAACGTTTAGTGAAAAAGGCGTGTCTTGGGGGGCTGAAG
AAGGTTATCATGACGATTTAGTAATGTCTTTAGTGATTTTTGGATGGTTATCAACGCAGTCAAAATTTAT
TGATTATGCGGATAAAGATGACATGCGATTAGCATCTGAAGTATTTTCAAAAGAGCTTCAGGATATGAGC
GACGACTACGCGCCAGTTATATTTGTGGATTCGGTTCATTCTGCTGAGTATGTTCCAGTATCTCATGGTA
TGTCAATGGTATAAATATATTAAAGCATATTAAAGAGGATTAAAAATGACTTTATTATCTCCGGGCATTG
AGCTCAAAGAAACTACGGTTCAAAGCACCGTAGTTAATAACTCTACTGGTACAGCAGCTTTGGCCGGTAA
ATTCCAGTGGGGTCCTGCTTTTCAGATTAAACAGGTTACAAATGAAGTAGATTTAGTTAATACTTTTGGT
CAACCAACCGCTGAAACTGCTGACTATTTTATGTCTGCGATGAATTTCTTGCAGTACGGAAATGACTTAC
GAGTAGTTCGTGCTGTTGATAGAGATACCGCTAAAAACTCATCGCCAATTGCTGGTAATATTGATTACAC
AATTTCTACCCCAGGTAGTAACTATGCGGTTGGAGATAAAATCACGGTCAAATATGTTTCAGATGATATT
GAAACTGAAGGTAAAATTACTGAAGTAGACGCAGATGGAAAAATTAAGAAAATTAATATTCCTACTGGCA
AAAATTACGCTAAAGCGAAAGAAGTCGGTGAATATCCAACACTAGGTTCTAACTGGACTGCGGAAATTTC
TTCATCTTCCTCTGGTTTAGCTGCAGTAATAACTCTTGGAAAAATTATTACTGATTCTGGTATTTTATTA
GCTGAAATTGAAAATGCTGAAGCTGCTATGACAGCGGTTGACTTTCAAGCAAATCTTAAAAAATATGGAA
TTCCAGGAGTAGTAGCGCTTTATCCAGGCGAATTAGGCGATAAAATTGAAATTGAAATCGTATCTAAAGC
TGACTATGCAAAAGGAGCTTCTGCATTACTCCCAATTTATCCAGGTGGTGGTACTCGTGCATCTACTGCT
AAAGCAGTGTTTGGATATGGACCGCAAACTGATTCACAGTACGCTATTATAGTTCGTCGTAATGATGCTA
TTGTTCAAAGCGTTGTTCTTTCAACTAAGCGTGGTGAAAAAGATATTTACGATAGTAACATCTATATCGA
TGACTTTTTCGCAAAAGGTGGTTCAGAATATATTTTTGCAACTGCACAAAACTGGCCAGAAGGCTTCTCT
GGAATTTTAACTCTGTCTGGTGGATTATCATCAAATGCTGAAGTAACAGCAGGAGATTTGATGGAAGCTT
GGGACTTCTTTGCTGACCGTGAATCTGTTGACGTTCAACTGTTTATTGCAGGTTCTTGTGCCGGTGAATC
TTTAGAAACAGCATCTACTGTCCAAAAACACGTCGTTTCAATTGGGGATGCTCGCCAAGATTGCTTAGTA
TTGTGCTCTCCTCCGCGTGAAACTGTAGTTGGAATTCCTGTAACCCGTGCTGTTGATAACCTAGTCAATT
GGAGAACTGCGGCAGGTTCATACACTGATAATAACTTTAATATCAGTTCAACCTATGCAGCAATTGATGG
TAACCATAAGTATCAGTATGACAAATATAATGATGTGAATCGTTGGGTTCCATTAGCAGCTGATATTGCT
GGTTTATGCGCAAGAACTGATAACGTATCTCAGACTTGGATGTCTCCAGCTGGTTATAATCGTGGTCAGA
TTCTTAACGTTATTAAACTTGCTATTGAAACTCGCCAGGCTCAGCGCGACCGTTTATACCAAGAAGCTAT
CAACCCGGTAACCGGTACAGGTGGTGATGGTTACGTATTGTATGGTGATAAAACAGCTACTTCTGTTCCT
TCTCCATTTGATCGTATTAACGTTCGTCGTCTGTTTAATATGTTGAAAACGAATATCGGACGTAGTTCAA
AATATCGTTTGTTCGAATTAAACAACGCGTTTACTCGTTCATCATTCCGCACAGAAACTGCCCAGTACTT
ACAAGGGAATAAAGCTCTCGGTGGAATTTATGAATATCGTGTAGTTTGCGATACAACAAATAACACTCCG
TCAGTAATTGATAGAAATGAGTTTGTTGCAACATTCTACATCCAACCGGCTAGAAGCATTAACTACATTA
CCTTAAACTTCGTAGCAACTGCTACTGGTGCAGATTTCGATGAGTTAACTGGTCTTGCTGGTTAATACGG
TGCATTCTAAAGGCCTGTTTCGGCAGGCCATATAAATACACTATATCCTTAATTCTTTAATTCTATATGC
CCTAGGTTAAACATAGGGATATAAATACTACAGAGGCTAATATGTTTGTAGATGATGTAACACGAGCGTT
TGAATCTGGTGATTTTGCTCGACCTAACTTATTCCAAGTAGAAATTTCTTATCTTGGACAAAATTTTACG
TTCCAATGTAAAGCTACTGCTCTACCAGCTGGTATTGTAGAAAAAATTCCAGTCGGATTTATGAACCGTA
AAATTAACGTAGCAGGCGATCGTACATTCGATGACTGGACTGTTACAGTAATGAACGATGAAGCTCATGA
TGCTCGTCAGAAGTTCGTTGATTGGCAAAGCATTGCTGCGGGGCAAGGAAACGAAATTACTGGTGGAAAA
CCTGCAGAGTATAAAAAGAGCGCTATCGTTCGTCAATATGCTCGTGACGCTAAAACAGTAACAAAAGAAA
TTGAAATTAAAGGTCTGTGGCCTACTAACGTGGGTGAACTTCAATTAGATTGGGATTCAAACAATGAAAT
CCAAACTTTTGAAGTAACTCTTGCTCTCGATTATTGGGAATAAAATGAATGGGGAGAAATCCCCATCCTG
CTTAAAGCAGAGAAGTCCATTATAAATATAACTATAATTCCCATTTGGAGAATACAATGAAATTTAATGT
ATTAAGTTTGTTTGCTCCATGGGCTAAAATGGACGAACGAAATTTTAAAGACCAAGAAAAAGAAGATCTT
GTTTCCATTACAGCCCCAAAGCTTGATGATGGAGCAAGAGAATTTGAAGTAAGCTCGAATGAAGCTGCTT
CTCCTTATAATGCTGCATTCCAAACAATTTTTGGTTCATATGAACCAGGAATGAAAACTACTCGTGAGCT
TATTGATACATATCGTAATCTCATGAATAACTATGAAGTAGATAATGCAGTTTCAGAAATCGTTTCAGAT
GCTATCGTCTATGAAGATGATACTGAAGTCGTAGCGTTAAATTTGGATAAATCTAAATTTAGCCCAAAAA
TTAAAAATATGATGTTAGATGAATTTAGTGATGTATTAAATCATCTATCGTTTCAACGAAAAGGTTCTGA
TCATTTTAGACGTTGGTATGTTGATTCAAGAATTTTCTTTCATAAAATCATTGATCCAAAACGTCCAAAA
GAAGGCATAAAAGAATTACGTAGATTAGACCCTCGCCAAGTTCAGTATGTTCGTGAAATTATAACAGAAA
CTGAAGCTGGCACAAAAATAGTTAAAGGTTACAAAGAATATTTTATATATGATACTGCCCATGAGTCATA
TGCATGTGATGGTAGAATGTATGAAGCTGGCACAAAAATAAAAATTCCTAAAGCTGCCGTCGTTTATGCC
CATTCTGGATTAGTCGATTGTTGCGGTAAAAATATCATCGGGTATTTGCATCGTGCTGTTAAACCTGCTA
ACCAATTAAAATTATTAGAAGATGCTGTAGTCATTTATCGCATTACTCGTGCTCCTGACCGTCGTGTTTG
GTATGTAGACACAGGTAATATGCCTGCTCGTAAAGCTGCTGAGCACATGCAACATGTTATGAACACGATG
AAAAACCGTGTAGTATATGATGCATCAACAGGTAAAATAAAAAATCAACAGCATAATATGTCTATGACCG
AAGACTATTGGTTGCAGCGCCGTGATGGTAAAGCTGTGACAGAAGTTGATACTCTTCCTGGTGCTGATAA
TACTGGCAATATGGAAGATATTCGTTGGTTTAGACAAGCTCTTTATATGGCATTACGTGTTCCTCTTTCA
CGCATTCCGCAAGACCAACAAGGCGGTGTGATGTTTGATTCTGGAACTAGCATTACACGTGATGAATTAA
CGTTTGCTAAATTTATTCGTGAGTTACAGCACAAGTTTGAAGAAGTTTTCCTAGATCCGCTTAAAACAAA
TCTTTTGCTTAAAGGTATAATCACAGAAGATGAGTGGAATGATGAAATAAATAATATTAAGATAGAATTT
CATCGGGATAGCTACTTTGCTGAGCTCAAAGAAGCAGAAATTTTGGAACGAAGAATTAATATGCTAACCA
TGGCAGAACCATTTATTGGTAAATATATTTCTCACAGAACTGCTATGAAAGACATTTTGCAGATGACTGA
TGAAGAAATAGAACAAGAAGCCAAGCAAATTGAAGAAGAGTCTAAAGAGGCTCGTTTCCAAGACCCCGAC
CAAGAACAAGAGGATTTTTAATGGAAGGTTTAATTGAAGCTATTAAATCAAACGACCTCGTAGCCGCTCG
TAAATTATTTGCTGAAGCCATGGCTGCAAGAACGATTGATTTAATTAAAGAAGAAAAAATCGCTATCGCT
CGCAATTTCTTAATCGAAGGTGAAGAACCTGAAGACGAGGATGAAGATGAAGATGACGAAGATAGTGATG
ATAAAGACGACAAAAAAGACGAAGACTCTGACGAAGACGAGGATGATGAATAATGCTTCTGATCCCTGAA
ACTCATGAATTAGTTCTCGAGAATGTCGAAGCACTTATTCCTGAAGCACAGGGTCGCTTTGACGAATTGT
CTTCTGCTTTAAATAAAGACGATATAAATACAATTGTCGAGAATATGCTTGATGATGAAACTGATTTAGC
GGTTGCATTAGCTTCTATTAATGAAAATATGCCGTTAAATGAATTCATCGTTAAACATGTTTCTGCCCGT
GGTGAAATTACTCGCACTAAAGACCGCAAAACGCGTGAACGAAATGCATTTCAAACCACTGGGCTGTCTA
AAGCAAAACGTAGACAAATTGCTCGTAAAGCTACCAAAACGAAGATTGCCAATCCAGCAGGTCAATCTCG
TGCTCAGCGTAAGCGTAAAAAAGCTCTTAAACGCCGTAAAGCATTAGGATTAAGCTAATGAATGAACCCC
AATTACTAATTGAAACTTGGGGTCAACCTGGCGAAATTATTGATGGCGTACCAATGCTTGAATCTCATGA
TGGAAAAGACTTAGGTTTAAAACCGGGTTTATACATCGAAGGAATATTCATGCAAGCGGAAGTCGTCAAT
AGAAATAAACGTCTTTATCCAAAACGTATATTAGAAAAAGCGGTAAAAGACTATATTAATGAGCAAGTTT
TAACTAAACAAGCTCTCGGAGAATTAAATCATCCTCCACGCGCTAATGTTGACCCGATGCAAGCCGCTAT
CATTATAGAAGATATGTGGTGGAAAGGAAATGACGTATACGGACGAGCTCGTGTTATTGAAGGTGACCAT
GGTCCTGGAGATAAATTAGCAGCTAATATTCGTGCCGGATGGATTCCAGGAGTTTCTTCTCGTGGATTAG
GTTCATTGACTGACACAAATGAAGGTTATCGTATCGTAAACGAAGGATTCAAATTAACTGTAGGTGTTGA
TGCAGTATGGGGTCCAAGTGCTCCAGATGCATGGGTAACTCCTAAGGAAATTACCGAATCACAGACGGCG
GAAGCCGATACAAGTGCCGATGACGCCTATATGGCTCTCGCAGAGGCCATGAAAAAAGCGTTATAAATAT
TATTATCTAAACAACAGGACTACAAAATGCTTAAAGAACAACTGATTGCCGAAGCGCAGAAAATTGATGC
TTCCGTTGCTCTTGATAGTATTTTCGAATCAGTTAATATTTCTCCGGAAGCAAAAGAAACTTTCGGCACT
GTATTCGAAGCTACCGTCAAGCAGCACGCCGTTAAATTAGCTGAATCTCATATCGCTAAAATTGCTGAAA
AAGCAGAAGAAGAAGTAGAAAAAAATAAAGAAGAAGCCGAAGAAAAAGCTGAGAAGAAAATCGCTGAGCA
AGCTTCTAAATTCATTGACCATCTTGCAAAAGAATGGCTCGCTGAAAATAAATTAGCAGTTGATAAAGGC
ATCAAAGCCGAACTGTTTGAATCCATGCTTGGTGGATTAAAAGAGCTCTTTGTTGAACACAACGTTGTTG
TTCCAGAAGAATCAGTTGATGTTGTAGCTGAAATGGAAGAAGAGCTGCAAGAACATAAAGAAGAATCGCC
TCGTCTGTTCGAAGAACTGAATATGCGCGACGCATATATCAATTATGTGCAGCGTGAAGTGGCATTGAGC
GAAAGTACTAAAGATCTGACTGAGTCTCAAAAAGAAAAAGTCTCTGCTCTGGTCGAAGGTATGGATTATT
CAGATGCATTCTCAAGTAAATTGAGTGCAATCGTAGAAATGGTGAAGAAATCTAATAAAGATGAAAGCAC
TATTACTGAGAGTATAAATACTCCTGATACTGAAGCAGCCGGACTGAATTTCGTCACTGAAGCTGTAGAA
GATAAAGCTGCACAGGGTGCAGAAGATATTGTAAGTGTATATGCGAAAGTCGCATCTCGTTTCTAATTTT
AAAGGTTAACACAAATGACTATCAAAACTAAAGCTGAACTTTTGAACAAATGGAAGCCATTACTGGAAGG
TGAAGGTTTACCGGAAATTGCTAATAGCAAACAAGCGATTATCGCTAAAATCTTTGAAAACCAGGAAAAA
GATTTCCAGACAGCTCCGGAATATAAAGACGAAAAAATTGCTCAGGCATTCGGTTCTTTCTTAACAGAAG
CTGAAATCGGTGGTGACCACGGTTACAATGCTACCAACATCGCTGCAGGTCAGACTTCTGGCGCAGTAAC
TCAGATTGGCCCAGCTGTTATGGGTATGGTACGTCGTGCTATTCCTAACCTGATTGCTTTCGATATTTGT
GGTGTTCAGCCGATGAACAGCCCGACTGGCCAGGTATTCGCACTGCGCGCAGTATATGGTAAAGACCCAG
TGGCTGCCGGTGCTAAAGAAGCATTCCACCCAATGTATGGTCCAGATGCAATGTTCTCTGGTCAGGGTGC
TGCTAAGAAATTCCCAGCTCTGGCTGCTAGCACACAAACCACAGTAGGTGATATCTATACTCACTTCTTC
CAGGAAACTGGTACTGTATATCTGCAAGCTTCTGTTCAAGTAACAATCGATGCTGGTGCGACTGATGCTG
CTAAATTAGATGCTGAAATTAAGAAACAAATGGAAGCTGGTGCACTGGTAGAAATCGCTGAAGGTATGGC
TACTTCTATCGCTGAACTCCAGGAAGGTTTCAATGGTTCTACCGATAACCCATGGAATGAAATGGGCTTC
CGTATCGATAAGCAAGTTATCGAAGCTAAATCTCGTCAGCTGAAAGCTGCTTACTCTATTGAATTAGCAC
AAGACCTCCGCGCTGTTCACGGTATGGATGCTGATGCTGAACTGTCTGGTATTCTGGCTACAGAAATTAT
GCTGGAAATCAACCGTGAAGTTGTTGATTGGATTAACTACTCAGCTCAGGTTGGTAAATCTGGTATGACC
CTGACTCCGGGTTCTAAAGCTGGTGTATTTGACTTCCAGGACCCAATTGATATTCGTGGTGCTCGCTGGG
CGGGTGAATCCTTTAAAGCTCTGTTGTTCCAGATTGACAAAGAAGCAGTTGAAATTGCTCGTCAGACCGG
TCGTGGTGAAGGTAACTTCATTATCGCTTCCCGTAACGTAGTTAACGTTTTGGCTTCAGTTGATACCGGC
ATTTCTTATGCTGCACAGGGTCTGGCTACCGGCTTTAGCACTGATACTACCAAGTCAGTATTTGCTGGTG
TTCTGGGTGGTAAATACCGCGTATATATCGACCAGTATGCTAAACAGGATTATTTCACTGTAGGTTATAA
AGGTCCGAACGAAATGGATGCTGGTATTTACTATGCTCCATATGTAGCTCTGACTCCGCTGCGTGGTTCC
GATCCGAAGAACTTCCAACCGGTAATGGGATTCAAAACTCGTTACGGTATCGGTATCAACCCATTTGCAG
AATCCGCTGCTCAGGCTCCGGCTTCTCGCATCCAGAGCGGTATGCCTTCTATTCTGAATAGCCTTGGTAA
AAACGCTTACTTTAGACGTGTATATGTTAAAGGTATCTAATCTCTAACGATAGAAACACAATTTTAGGGA
ACCTTCGGGTTCCCTTTTTTCTATTTTATACGATAGCAATCAGGCATATCATCCGCATTTATCCAATTGC
GAATAGTTTTAGGACTAACTTTAAAATGCTCCGCTGCGTAATCAGGATTATCAAATTTAACGCCCTTTAT
ACATATTGGAATAAATTTTTTAATACCACCAAGTTTTTCAGAAATAGCTTTACGATGTGAAATCGATATA
GGTTTGTTTTTTCGTGGATGAACATGTGTTTTATAATATTCATTGCGCCCTTTAACTCGCTTCGCAATAG
TTTCATCAGATTGCTTAACGCCTGTTTTTGCCTTTGATATTTTTCGTTTAGCTTCCACAGTCATTCCTTC
TTTTGTTCGTATTGATAACATATTACGATATGAAGGATCTTGCAAATGAACTATAACTGGATTTCCTCTG
CCACCAATAGCAGCATTATAGGTATCAGTTCTCATAACGAATTCCTCATTAACTAGTAAAGCTTCCATTT
TATACATCTCCTCAGATGAGGAGAAAGAATAAAGAATTTCTTTTTTAAAGTTATGAATACCATATTTTTT
GATGGATTTTTTGATGTTTACGCCAGAACCCATATAACCATCGTTTTCGTCAAGAGTAGCATGAGCTCCG
ATGTAAATTTTTCCATTGATGATATTAGTAATTTGATATATTAAATATTTCATTTTAAACATCACTCCGT
TTGTATATGATTATAATATCATATTACTTTGGTCTTGTAAATAACTTTATAAATAGTATTATATTTCAAC
AAGGAAAATACAATGGCTAAAATCAACGAACTTCTGCGCGAATCAACCACAACGAATAGCAACTCAATCG
GTCGCCCAAATCTCGTTGCTTTGACTCGCGCTACCACTAAATTAATATATTCTGACATTGTAGCAACGCA
AAGAACTAATCAACCTGTTGCTGCTTTTTATGGTATCAAATACCTTAACCCAGACAACGAATTTACATTT
AAAACTGGTGCTACTTACGCTGGCGAAGCTGGATATGTAGACCGAGAACAAATCACAGAATTAACAGAAG
AGTCTAAATTAACTCTCAATAAAGGCGATTTATTCAAATATAATAATATCGTTTATAAAGTATTAGAAGA
TACTCCATTTGCTACTATCGAAGAAAGTGATTTAGAATTAGCTCTTCAGATTGCAATCGTTCTTTTAAAG
GTTCGTCTATTTTCTGACGCAGCGTCAACAAGCAAATTTGAAAGCTCTGATAGTGAAATTGCGGATGCTA
GATTCCAGATTAATAAATGGCAAACTGCAGTTAAATCTCGTAAACTTAAAACTGGCATCACAGTTGAATT
AGCGCAAGATTTAGAAGCAAATGGATTCGATGCTCCTAATTTCTTGGAAGATTTGCTTGCAACTGAAATG
GCAGATGAAATCAATAAAGACATTCTGCAGTCTTTGATTACAGTGTCAAAACGCTATAAAGTTACAGGAA
TTACTGATAGTGGATTCATCGATTTGAGTTATGCATCTGCTCCTGAAGCTGGTCGTTCATTATACCGAAT
GGTATGTGAAATGGTTTCGCATATCCAAAAAGAATCAACTTATACAGCAACGTTCTGTGTTGCTTCAGCT
CGTGCCGCTGCGATTCTTGCTGCATCAGGCTGGTTAAAACATAAACCAGAAGATGACAAATATCTTTCAC
AAAATGCCTACGGGTTCTTAGCTAATGGTTTACCGCTTTATTGCGATACTAACAGCCCATTAGATTATGT
AATCGTTGGCGTAGTAGAAAATATCGGTGAAAAAGAAATTGTTGGATCAATTTTCTATGCTCCGTATACA
GAAGGTCTCGACTTAGATGACCCTGAACATGTAGGTGCATTTAAAGTTGTTGTTGATCCAGAAAGCTTAC
AACCATCTATCGGTTTATTAGTTAGATATGCTTTATCAGCAAATCCTTATACTGTAGCAAAAGATGAAAA
AGAAGCAAGAATAATTGACGGTGGAGACATGGATAAAATGGCAGGTCGTTCAGATTTGTCTGTTTTATTA
GGTGTTAAGCTACCAAAAATTATCATTGATGAATAAAACAAAGGGACCTTTCGGTCCCTTTTTATTTAAC
TTACCAACTCAATCCAAGCTGGACGAAGTACATCTTGTACCATTTTAACTAATTCCTTTTTAATCAAAGA
AGGATTATCTGCTTGAGTTAGAGTAATACCTTCACGAGAAGTTTCTTCCAAAATATCTTGAACAGTTAGC
CCCATCACCTTTCCAAAATCCTTTGGACCAATTTCGCCAATTTTAGAAATAACGTTATTTACGCGGTTCA
GTGTAACGTAACAAGCTAAAATTCCCACCAATTTGTTATCAGCTTCTGATAGCTCAACTTTAGCTTTAAT
AGGCTTATCAGACTTTTTCTTTTCACTAAATTTAGAGTTCTTGCATTTAATCGCTACACGATTTCCATTA
CGAAGCCAAGAAGGATAACAAGGTTTCAATACATATCCTTCAGCAGTAAATACTTCGCCTTTTGCTTCGG
CATTCCAAACGCATTTATTTGCATCAACTAATCCAGCATGGTCTACTGTAAAATTATAATCTTGGACGAC
AGAATCTAAATCATTTGGCAATTTAATAAGCTCTTCAAATTTACCGCGACCTAAAAGTGGAGCCATTTTA
AATTTAAATGTATTACAGAATGATTCCATCATATAATCATCTACATAAGTCACATCACCGCTTTCTGTAG
TAACAATAATGTCAAATACATAAAAATCTTTATCACAATAATCAACATTCTTCTGAATGCCAGGTCCAGC
GAATTCGCCAAAGACTTGATAAGATACAACCGCTGAGGTTTCCATAATATCTTGTACAGCTTTAATGGAA
TCAGCATAATTCTTCAAAATAATTTCATACCCAAAGAAATCTTCAGCAGGAAGAATCGGTCCAGTGCGTT
TAGCGCAAGTCACTTTATCACGCTCAATAATCAATGAGAAATTTGTGCCGTGAATCTTTTCACGAGCTAC
CCACTCCCCACCAGTCAATCCCAAGCTATAAAGTTTTTCAATAAATTTAGAGTTGTAATGATTTTCAAGA
CTGCTATACTTTTTAAACATAATTAATCCTCAAAATGTAATTTCTAACCAATCACCATCACGCTGATCAC
TATTGACTTTAAAGCTGAATCCTTCTTTTCTCAGCCAATCACCAATTTCTTCTGTAATCAATTTATCACG
AGCAATACAATAATAATTAAAATGTGTTTTACCTTGTTCAGCTGCTTTATTAGCAAGTTCTGAAAAATCT
TTAATAAAACACTCTAGCTTAAACTGTTTACTTTTTAATGCTTTTTCGCGTAATTGATTAGCAAAAGATT
CATTTTCATAAAGATCATACTGTTCCATTTTTCACCTTTTTATTGATATGTCTTTTTCTATAGACAACTT
TTTCTCGAGCCCATAATACAGCCACTTCTTTTGCCTGTAAGTTTAATTCACGAGCAATTTCAATGAATGA
CTTTCCAGACTCATGAAGAGTAAACACCACAACCTCAGTTCTCATAATCAATCTCATGTTATCGAGTTGG
TGCCATTATATACATCATTTTCTGATTGTGTTTTGTGTGCTTTCAAAATGAAGAAAGGGGCCGAAGCCCC
TTATGATTATGGATAGGTATAGATGATACCAGTTTCTAAAGCAGTTTTATGAATGATGTATCCATTACGC
GATTCTTGGACATCAACTTCTGGATAGTCTTTCATCATCTTCTGGAGAGTGTAACGATGCAGGTAATATT
TACTATCTGGGTCGTCAGTTTTCCAATCTTTACCTTCTTCGGTCATTTTTTGGATTTCATCCATAACCCA
CCAACCGCACCAGATGTAAGCTGAGCTACGGTGTGGAAGAGGATGAACATAAGGTAATTCACCTTCTGGT
TCTGGAGCAACTTTAGCCGTGACTGTTACATTACCAGTTTTGGTAACGGTTACAGGGTTATAATCTGCAG
CAGTAACAGTTGCAGTAACTTCAATAGTTTGACTTCCAACAGATGAGGTATCGACAGTATATACGTTAGT
TGACCCTTCTACAGGAGAAGAATCTTTCTTCCATGAGTAAGTAATTTGTGCTTCTTCTGGAGCACCCGTA
ACATTAGCCGTAAATGTAGCCGAAGCATCTTGCTGAACATTAATAGAAGGAGGAGTCAATGTAACCTGTG
GATTCATTGTCTTCTTATTAACCGTTAATGATACTTCATTAGAAGTAACGCTTAGTGCATCATAATCTGT
CGCGGTTACTTGGGCTACGCATTTAATTCTTTTTACTCCACTTGTAGTTGGAGTATAGCTAAATGTAGAG
TTAGTTTCTCCACCAACTTGTGAATCATCTACATACCACTGATACGTAGCAGATGCTCCATCAGGTTGAG
AAGCTAAGGCAGCAGTAAATTGAACTGGGGTTCCAATCACTCCAGCCGCAGGACTAGCAGGAGTTACGGC
TAAGGTAGTCGTCTGTGTCTTATTTTTAACTGTGATAGTTGTTGTCGCTTCAGCCGTTTCCGGGCCTCCT
TCAGAAAGTGTATTTGTTGCAACTACTTTAATAGTCTTTTGACCGGCAGGTCCTTTTAGTACATAACTAA
AAGTTGCTTCAGCTCCATCTTGTGGAACATTATCTACGCTCCAAGCATATGTAATAGTTCCGCCTCCAGT
TTGACCACTGGGTGTAGCAGTAAACTGCTTAGTTTCATCAATAACCCCTGTAGGTGTTTTAGGAGTTATA
TCAACTGTAAAAGTCATAAGTTATCCTTATTTTAATGTTACGAAAGAAGAGTTGCGTGTTTCACGAATTA
AAACTGATCCATCGCGATTAATGTAATAAATTAAGCTAAATAAAGTTTGGTGTGCTGACGCATGTTCAAA
ACTAGTTGGGTGAGATTTCCAATCAGGAGTTTCAGCAATCCATTGATAAATCCACCAAGGAACAGTACAG
AATCCTAGATTTTTTCCAATCAGTTGAAGATTCGGACTAAAGTTTTCCGGAAGAGTAAATACAGACGGCT
TTTCAGATTCAATAATCTCAGCTACAGCCTGTTCGAATTTTTCTTCAACAAAAGGAGTATCTTCAATCAA
AACATCGGTATTTTCAGGAATTTTATCCGTTTCTACTACTTCAATTTTAATGTCAGATTTAATCGGAGAA
TCAATCAGAAGTGCTGCTTCTGGATTGACTTCTTCATCGTCATATTTTAATCCCTCTGCGGCATCAGCAG
CATCAATTAAGTCTTTAATAGATAACCCATCAGTCTCTGGCATAGGTTCACTAGCGAGCTTCTGGAGGGC
TTCTTCAATATCAACAACGATATTATCAAAAGATTTATTCTTTTTGACCETTATACCAAACTGTTCAGCA
TATTCAGCTAATTTAGCTTTAGCTTCTTTGTTATCATCAAGAGCCTTCAGCTCTGCAATATAATCTTTAT
CTATCATAATATTTCCTCAGTATAAATATAGATATATTTATTACTCGGAAAATAGTATGTACCACTTTGT
ATATGAAACAACAAATCTAATAAATGGTAAAAAGTATATAGGAAAGCACTCTACTGATGACTTGAATGAT
GGTTACCTTGGTTCCGGTAAGGCAATTCAGCAGGCTATAAAGAAATATGGTGAAAACAATTTCTCTAGAA
CAATACTAAAAGAGTTTAAAACTTCCGAAGAAGCGTACATGTATGAAGAAGAAATTATAACTCCTGAACT
AATAAAAAGCAAAAATTATTATAATATGAAACCTGGTGGAATTGGTGGAATTGTTATGACTACAGATGTT
ATAGCAAAGATGAAAGAATCTTCCGCTAAAAGATTTGAAAACTCACCGGGCACGGTATTAGGTAAAACTT
GTTATACTAATGGAACTAAAAATATTTTTATTAAACCTGGAGAACTTGTTCCAGAAGGATTTGTAAAAGG
GATGGTTCATCCTAATAGAAAGTCCAGAAAAGGATGTAAAGTCAAACCGACTACCACAGGAACTTTTTGG
GTCAATAATGGCGCAATAAATAAATTAATACAACCAGACGGTATTATTCCCGACGGATTTATTAAAGGTC
GTCTCATGAAAAGAGATTCTAAAGGCAAATTTAGTAAGGCATAATTATGGATATTAAAGTACATTTTCAC
GACTTCAGTCATGTACGCATCGATTGTGAAGAGAGCACGTTCCACGAATTAAGAGATTTCTTTTCGTTTG
AGGCCGATGGATATAGATTTAATCCTCGCTTCAGATATGGCAACTGGGATGGACGAATCCGTCTTTTAGA
TTATAATCGTCTTCTTCCATTCGGCTTAGTCGGGCAAATTAAAAAATTCTGTGATAATTTTGGCTATAAA
GCCTGGATTGACCCACAAATTAACGAAAAAGAAGAATTATCAAGAAAAGATTTTGATGAATGGCTTTCTA
AATTAGAAATCTATTCAGGAAATAAAAGAATTGAACCGCACTGGTATCAAAAAGATGCAGTGTTCGAAGG
ATTAGTTAATCGTCGTAGAATTCTTAATCTTCCAACATCTGCAGGTAAATCTTTAATTCAAGCTTTGCTT
GCGCGATATTATTTGGAAAATTATGAAGGTAAAATTCTTATCATTGTTCCAACAACTGCTCTGACAACTC
AGATGGCTGATGACTTCGTCGACTATCGTTTATTCAGCCATGCAATGATAAAGAAAATTGGTGGCGGAGC
ATCAAAAGATGATAAATATAAAAATGATGCACCAGTCGTTGTTGGTACATGGCAAACTGTAGTAAAACAA
CCGAAAGAATGGTTCTCACAGTTTGGAATGATGATGAATGATGAATGCCATCTTGCTACAGGAAAAAGTA
TTTCATCTATCATATCAGGTTTAAATAACTGCATGTTCAAATTCGGTTTGTCTGGTTCATTACGTGATGG
CAAAGCCAATATCATGCAGTATGTTGGAATGTTTGGTGAAATATTTAAACCAGTAACGACTTCTAAATTA
ATGGAAGATGGACAAGTAACTGAGCTAAAAATTAATAGTATTTTTCTTCGCTATCCCGATGAGTTCACTA
CTAAATTAAAGGGAAAAACTTACCAAGAAGAAATAAAAATTATTACTGGGCTTAGTAAAAGAAATAAATG
GATCGCTAAATTAGCTATTAAGCTTGCGCAAAAAGATGAAAACGCTTTTGTCATGTTTAAACATGTATCG
CATGGTAAAGCTATTTTCGATTTAATTAAAAATGAATACGATAAAGTTTATTACGTATCAGGGGAAGTTG
ATACCGAAACCCGCAATATAATGAAAACCTTAGCTGAAAATGGTAAAGGAATAATTATAGTAGCTAGTTA
TGGTGTATTTTCTACTGGTATTTCAGTTAAAAATCTGCATCACGTTGTTTTAGCGCACGGTGTTAAATCA
AAAATCATTGTATTACAAACAATCGGTCGTGTATTACGTAAGCATGGTTCTAAGACAATAGCAACAGTCT
GGGACCTCATAGATAGCGCAGGCGTCAAGCCAAAATCTGCTAATACGAAAAAGAAATATGTTCATTTGAA
CTATCTTTTAAAACACGGCATTGATCGTATTCAGCGCTACGCAGATGAAAAATTTAATTACGTAATGAAA
ACAGTTAATTTAATAAGCTTCGGCCCTTTGGAGAAAAAGATGTTACTAGAATTTAAACAATTTCTTTATG
AAGCTTCTATTGATGAATTTATGGGTAAAATTGCCTCTTGTCAAACATTAGAAGGTTTAGAAGAACTTGA
AGCTTATTATAAGAAAAGAGTCAAAGAAACTGAATTAAAAGATACTGATGACATCTCTGTGAGAGATGCT
TTGGCAGGAAAAAGAGCTGAATTAGAAGATTCAGACGATGAAGTAGAAGAAAGCTTTTAAATTAAAAAAG
GCCCAACCAAAAAGGAAGGGCCAAAACTATAGACTAAAGGTCACACTATAGCAAAAGTTGTGTTTCATTT
AATTGTTCTTCCGAACTTTCTGAAACTGGTAGTTCTTTAATGTAATTATAGCAAGGCCCAGGATGTACAG
GACCTTTGTCTGTTTCAACAACCAATGCAGAATCGATTGGAGTTTTACAGACAACACAAATCTTATCTGA
CATGATTGTCTCCTCTGAATTATATCTATTTATACAACTCTCATATGCATATCAATGCCCATATCTTTAG
AATAAAAATATTCATCAAGATATCCGGCAAATTTTCCTTTAATATAAAGGACATCTTCACCACACGGGTG
GTCGGCCAGGATACGAATATCCTGACGCTTAAGATTATGCTTTTTCATTAAGAATTGAATTTCCGTTTCA
AATTCTTCTTCATAATTAAAAGCATCATCAATGCTATATCTCATTATTTTCCAGCCTCAAATGCTCGCAT
GTCTTGAATATGCTTAATAGCAAATCCACGTGATTTAATAGCATCAAGAGCTCCGCTACAGAAATCTAAT
AAAATCCCCCAATACTGCAACGAGGTATCAACCTTTAAAACATCCTTATCCGCTGATAGAACTGTCTTCA
TTTCTGATTTCTCGTAACGATCCATACTAAATTCATCACCATCTCCTCGTCCCGAGTAGTAGTCTAATCT
AGCTTTAAGAGCAACTTTTTTCTGTGCTTCAATTCTAAGCATTTCCTTTTTAATACTTGAATGCTTATTA
AGCCATTTACTATATAACATCACATTATTAGCTGCTTCATACTGTAATTTAGTCGAATCTATAAACACAT
CTTTCTTCAATTCTTCTTGAAGATCTTCTAATCTCATATTGTTCTCTATTCAATTGTTATTGGTTGTTAT
TGGATGGACTTAGATTCATTATACCACGTTTTAACGTGAAGCATTATACTCTATTACTGGAAGCCAGCTG
CAGTTTTATCTGCTCAATATCATCAGGATTATCGATGACCGAAAAGCGTATTTCTACTATCAGAGTATAA
TCGTCATAAACGGGTATCACATTAACTGCTAATTTATCAATACGTGGCTCATAGTTTCTTACTGCGCTTT
CGATATTGCGTTCAACCGTGTCAGCAGTAAGAGGAGTCATATTTTCAAAAAGCTGGTCTGATAAATCACA
TCCAAATTCAGGGTCAAACGGTCTTGAACCTTTTCTTGTTGTAATAATTCCCAAAAGACTGTTTTTAATT
GACCTTAATCCAAGCGATCTGGAAACGTCTTTGTTCCAATCCATTTTCATTTCCGGGTCAATATCAGAAT
AAAGCTTATTAATATTTGCCATTATAGTAACTCAAAGAACTCTTTGAGGCCTCTTATTACGTGAGCATGG
GTTTTTCCACACTCTGGACACTTAATTGGAACAGCCAAATAAACGGTAGGCTTTAAAAGCATATCTTTTA
TAGCTACAATATCTGACTCTGTGATGATAGAATATAAATCTTCTAGTTCCTTTTCATTTAAGTCTTCAAC
TGGAATGCTTTCCCCGTTAGCATGAATCGTTTCTATACATGATACTATCATGTGGGCTATATTTTTATCA
TCAAAAATTTTAGGGTATCGGAATTTAATTTTAATGTCACCTAGTGTATACCAGAGGTCTTCTGGTGCAT
CTATTTGTGTATGTAATAGATTTATATGGGTTGGTATTTCAGTTCCACAGGTGCACTTCCAGGAGTTTTC
GTGATTAACTTCACCGAGAGAATGTGCCCATAAATGAATCAACAATAGTTCTGATTCTTGGCGGTTTAAA
TCTTTTGCATTTGTGCAGTCTTTGATTAGCTTTTTAACAATTACTTCTACGGAACCATTATTTTTGGCAG
TAATAAGTTCTAGATATTCTTTAAGCGTGAATGCGCGACAATTGATTATTTTAGAACCAACTCTCACATC
AAATTTGTATTCATACATATTTAGCTCCTTTATTTATCATATTTATAAATAGAATAAAAGGAGCATCTAT
GGCAAACATTATTCGTTGTAAATTACCAGATGGTGTTCATCGTTTTAAACCATTTACGGTAGAAGATTAT
CGAGATTTTTTGTTAGTTCGAAACGATATAGAACATCGGTCACCACAAGAACAAAAGCAAATAATTACTG
ATTTAATTGATGATTATTTTGGAGACTATCCGAAGACTTGGCAACCATTTATATTTTTGCAGGTATTTGT
AGGGTCAATAGGTAAAACTAAAAGTACGGTCACATTTATATGTCCAAAATGTAAAAAAGAAAAGACAGTT
CCATTTGAAATATATCAAAAAGAATTAAAGGACCTTGTTTTTGATGTAGCTAATGTTAAAATTAAATTAA
AGTTTCCTTCTGAGTTTTATGAAAATAAAGCAAAGATGATTACTGAAAATATTCATTCTGTTCAAGTAGA
TGAAATATGGTATGATTGGAAGGAAATTAGCGAGTCCAGTCAAATAGAACTAGTTGACGCCATCGAGATA
GAAACATTAGAAAAAATTCTCGATGCAATGAATCCTATTAATTTAACTCTACACATGTCATGCTGTAATA
AGTACATTAAAAAATACACTGATATAGTAGACGTGTTTAAGCTATTAGTTAACCCAGATGAGATATTTAC
TTTTTATCAAATTAATCACACACTCGTAAAAAGTAATTATAGCTTAAATTCAATAAGTAAAATGATTCCT
GCCGAGCGCGGATTCGTATTAAAACTGATTGAGAAGGATAAACAATAATGAGTATGTTGCAACGCCCCGG
ATATCCAAATCTCAGCGTTAAATTATTTGATAGCTACGACGCTTGGAGTAATAATAGATTTGTTGAATTA
GCTGCTACTATTACCACATTAACTATGCGGGATTCTCTTTATGGCCGAAATGAAGGAATGCTGCAGTTTT
ATGATTCTAAAAACATCCATACAAAAATGGATGGAAATGAAATAATTCAGATTTCTGTAGCTAATGCAAA
TGATATTAATAATGTTAAAACACGAATTTATGGATGTAAGCATTTTTCCGTGTCAGTAGATTCAAAAGGT
GATAACATCATTGCTATTGAATTGGGAACTATTCATTCTATAGAAAATCTTAAATTTGGTAGACCATTTT
TCCCTGATGCAGGTGAATCTATAAAAGAAATGCTTGGTGTCATTTATCAGGATCGCACATTATTAACTCC
AGCAATAAATGCTATAAATGCTTATGTTCCTGATATTCCATGGACTAGCACATTTGAAAACTATTTGTCA
TATGTAAGAGAAGTTGCTCTAGCTGTAGGAAGCGACAAATTTGTATTTGTATGGCAAGACATCATGGGCG
TTAACATGATGGACTATGATATGATGATAAATCAAGAACCATATCCAATGATTGTCGGTGAGCCATCTTT
AATAGGTCAATTCATCCAAGAATTAAAATATCCATTAGCATATGATTTCGTTTGGTTGACTAAATCGAAT
CCTCACAAACGTGACCCAATGAAAAACGCTACTATCTATGCGCATTCATTTTTAGATTCTTCAATACCAA
TGATTACTACAGGAAAGGGTGAAAACTCTATTGTGGTGTCAAGGTCAGGTGCTTATTCTGAAATGACTTA
TAGGAATGGATATGAAGAAGCTATTCGTCTTCAAACTATGGCACAATATGACGGCTATGCTAAATGTTCT
ACTATCGGTAATTTTAACTTGACTCCTGGTGTTAAAATTATTTTTAATGATAGTAAAAACCAATTTAAAA
CAGAATTTTACGTTGATGAAGTTATCCATGAATTATCCAATAATAATTCAGTAACTCATCTATATATGTT
CACTAATGCAACGAAACTGGAAACAATAGACCCAGTTAAGGTTAAAAATGAATTTAAATCTGATACTACC
ACTGAAGAAAGTAGTTCTTCCAATAAGCAATAAAGAAGTTTCTATTCCTAAAATGGGTCTTAAACATTAT
AACATTTTAAAAGATGTTAAAGGTCCTGATGAAAATTTAAAACTTCTCATTGATTCTATTTGTCCGAATT
TATCACCGGCAGAAGTTGATTTCGTTTCTATTCATTTATTGGAATTTAATGGAAAGATTAAATCTCGTAA
AGAAATAGATGGTTATACTTATGACATTAATGATGTTTATGTATGCCAAAGATTGGAATTTCAATACCAA
GGAAATACATTTTATTTTAGACCTCCTGGAAAATTTGAACAATTTTTAACGGTGAGCGATATGTTATCTA
AATGCTTACTTAGGGTCAACGATGAAGTTAAAGAAATTAATTTTCTTGAGATGCCAGCATTCGTTTTAAA
ATGGGCAAATGATATTTTTACAACTTTAGCAATTCCTGGCCCTAATGGTCCAATAACTGGAATTGGCAAT
ATTATTGGATTATTTGAATGAAAAAGCCACAAGAAATGCAAACGATGCGTAGAAAAGTTATTTCAGATAA
TAAACCAACACAGGAAGCGGCTAAATCCGCTTCTAATACTTTATCTGGGCTTAATGACATATCTACGAAA
TTGGATGATGCTCAAGCTGCTTCTGAATTAATAGCTCAAACTGTCGAAGAAAAATCGAATGAAATAATTG
GAGCAATTGACAATGTAGAAAGCGCAGTGAGTGATACATCTGCCGGTTCTGAGTTAATTGCTGAAACTGT
CGAAATTGGCAACAATATTAATAAAGAAATCGGTGAATCGCTCGGAAGCAAATTAGATAAATTAACAAGT
TTACTAGAGCAAAAAATCCAGACAGCTGGAATTCAACAGACTGGAACTAGTTTAGCTACGGTTGAAAGCG
CTATTCCTGTTAAAGTCGTTGAGGATGATACTGCTGAATCTGTGGGTCCTTTATTACCAGCTCCTGAAGC
AGTTAATAATGATCCTGACGCTGATTTTTTCCCTACCCCTCAGCCAGTTGAGCCAAAGCAAGAATCACCA
GAAGAAAAACAGAAAAAAGAAGCATTTAACTTAAAATTATCTCAAGCTTTAGATAAATTAACGAAGACTG
TTGATTTTGGATTTAAAAAATCCATTTCAATTACTGATAAAATATCAAGCATGCTATTTAAGTACACCGT
CAGTGCTGCTATTGAAGCTGCTAAAATGACTGCAATGATATTGGCTGTTGTTGTTGGAATAGACCTTTTG
ATGATTCACTTTAAATACTGGTCAGATAAATTTTCAAAAGCCTGGGATTTGTTTAGTACAGACTTTACCA
AATTCTCTAGCGAAACCGGAACTTGGGGTCCTTTATTACAGAGCATCTTTGATTCTATTGATAAAATTAA
ACAACTTTGGGAAGCGGGAGATTGGGGTGGATTGACAGTAGCTATTGTTGAAGGGCTTGGAAAGGTTCTT
TTTAATTTAGGTGAACTTATTCAATTAGGTATGGCTAAATTATCTGCAGCAATTCTTCGAGTCATTCCTG
GTATGAAGGATACTGCTGATGAAGTAGAAGGAAGAGCATTAGAAAATTTCCAAAATTCTACTGGAGCATC
TCTCAATAAAGAAGACCAAGAAAAAGTAGCAAATTATCAAGATAAACGAATGAATGGAGACCTTGGCCCA
ATAGCAGAAGGACTAGACAAAATCTCTAACTGGAAAACTCGTGCATCTAACTGGATTCGTGGTGTAGATA
ATAAAGAAGCGCTGACTACCGACGAAGAGCGTGCGGCAGAAGAAGAAAAATTAAAGCAACTTTCACCGGA
AGAAAGAAAAAATGCTTTAATGAAGGCTAATGAAGCTCGTGCTGCGATGATTCGTTTTGAAAAATATGCC
GATTCAGCTGATATGAGTAAAGACTCAACGGTTAAATCAGTTGAAGCTGCCTATGAAGACCTTAAAAAAC
GGATGGATGACCCGGATTTAAATAATTCACCGGCAGTTAAAAAAGAACTTGCTGCTAGATTTTCTAAAAT
TGATGCTACTTATCAAGAGCTCAAGAAAAATCAGCCTAATGCCAAACCTGAAACTTCTGCTAAATCACCA
GAAGCGAAACAAGTCCAGGTGATTGAAAAGAACAAAGCACAGCAAGCTCCTGTTCAACAAGCATCTCCTT
CGATCAATAATACTAATAATGTTATTAAGAAAAATACTGTCGTTCATAATATGACACCTGTAACGAGCAC
GACTGCTCCTGGTGTATTTGATGCGACTGGAGTTAATTAAGGAATAATATGGCAATTGTTAAAGAAATAA
CTGCTGATTTAATTAAAAAGTCCGGTGAGAAAATTTCAGCCGGACAGAGTACTAAATCAGAAGTAGGAAC
TAAAACATACACAGCCCAGTTTCCAACTGGGCGTGCTAGTGGTAATGACACTACAGAGGACTTCCAGGTA
ACAGATCTATATAAGAATGGATTATTATTTACTGCATACAATATGTCATCTAGGGATTCTGGAAGTCTTA
GATCGATGAGATCTAACTACTCTTCTTCATCTTCGAGTATTTTACGTACAGCTAGAAACACTATTAGTAG
TACAGTATCAAAACTATCAAATGGATTAATATCAAATAATAATTCAGGAACAATAAGTAAATCTCCTATC
GCAAACATTCTTTTACCGAGATCTAAATCTGATGTTGATACATCATCACATAGATTTAATGATGTTCAAG
AAAGCCTTATCAGTAGAGGCGGAGGTACTGCTACTGGTGTGCTAAGTAATATTGCTTCAACCGCAGTATT
TGGGGCACTGGAAAGTATAACACAAGGTATAATGGCTGATAATAATGAACAGATTTATACGACAGCCAGA
AGTATGTATGGTGGTGCTGAAAATAGAACTAAAGTGTTTACATGGGATTTGACTCCACGTTCAACAGAAG
ATTTAATGGCTATTATTAATATCTATCAATATTTTAACTATTTTTCTTATGGTGAAACGGGTAAATCTCA
ATATGCTGCTGAAATAAAGGGGTATTTAGATGATTGGTATCGTTCTACGTTAATTGAACCTTTATCTCCG
GAAGACGCAGCTAAAAATAAAACACTATTTGAGAAAATGACATCGAGTTTAACTAACGTTCTAGTAGTTT
CAAACCCGACAGTTTGGATGGTGAAAAACTTTGGCGCAACATCTAAGTTTGATGGAAAAACGGAAATATT
TGGTCCATGTCAAATACAGAGCATTAGATTTGATAAAACACCTAATGGTAACTTTAACGGATTAGCTATT
GCTCCAAACCTCCCTAGTACATTTACTCTCGAGATTACTATGAGAGAAATTATCACGTTAAACCGTGCTT
CTTTATATGCGGGGACTTTTTAATGTATTCTTTAGAGGAATTTAATAATCAAGCAATAAACGCAGATTTC
CAACGTAATAATATGTTTAGCTGCGTTTTTGCGACAACTCCATCAACTAAAAGCTCTTCGTTGATAAGTT
CAATTAGCAACTTTTCTTATAATAACTTGGGCCTAAATTCAGATTGGTTAGGATTAACTCAAGGTGATAT
TAATCAGGGAATTACCACGCTAATTACAGCTGGCACACAAAAACTGATAAGAAAATCAGGAGTCAGTAAA
TATCTTATTGGTGCCATGAGTCAACGTACAGTTCAAAGTTTATTAGGCTCATTTACAGTTGGTACATATT
TAATTGACTTCTTTAACATGGCATATAACTCATCTGGATTGATGATATACTCTGTAAAAATGCCAGAGAA
TAGATTATCCTATGAAACTGACTGGAACTATAATTCTCCTAATATTCGTATAACCGGAAGAGAATTAGAC
CCTTTGGTTATTTCATTTAGAATGGATTCAGAAGCTTGTAACTATCGTGCAATGCAAGACTGGGTTAACT
CCGTTCAAGACCCAGTAACTGGACTGCGTGCTTTGCCACAAGATGTCGAGGCAGATATTCAGGTTAATCT
TCATTCTCGCAATGGATTACCTCATACTGCGGTGATGTTCACGATGCATTCAATATCAGTGAGCGCTCCT
GAGTTATCATATGATGGAGATAACCAAATAACTACATTTGATGTTACTTTTGCGTACAGAGTGATGCAGG
CTGGAGCAGTTGATAGGCAACGTGCGCTTGAATGGCTTGAATCTGCTGCTATAAATGGTATTCAAAGCGT
TCTCGGAAATAGTGGAGGTGTTACTGGACTATCTAATTCGCTTTCACGACTTAGTAGATTAGGGGGAACT
GCAGGAAGCATTTCAAACATTAATACTATGACAGGAATTGTCAATTCGCAGAGTAAAATATTAGGAGCAA
TATAACAATGGGGACCGAAAGGTCCATATTTTTATTTACGGAATGAAATGAAAGCAGCAACTGAAGCAAC
TAAACTGTCTTCAATATAAACTTCAATTTTTACAGGAGCTTCTGACTCAAATTTACCTGTTACTACACCC
TGAAAAATACTTTCAGTCTGTTCTGGCTTTGAAAAATTTTCAGAAGGAAAAATTCCGAACTTTTTATCTG
TTCCAAAAATTTTGATAAATTCATCGTAAACCGCTTCGTTAAAAGCATTATCAGCAGGAATAACGCCTTC
AACTACAAGTTCTTGACCTAAAAAGCGTAAAGAAGATTTCATTTTGTGTTCCTCATGTTATGTTAGTAAG
ACTACTATAACACAACACGAGGGACTTGTAAACTACATTTTGAACTTTTTAGTACGCGTAATAGGCATGC
GTCGATTTTATACTGTTTCATTGTTTGAAGAGCAGTATCAAAAACAGCATTAATGACACCAATTGGATTT
CCACCCAAGTTTTTAAGCTTAACTAATGATAGTTTCTCATTAACACCTATCATTACGATATGCATCATTT
TATCGCCTGGTTTAACGTTTTATTTGTATCACCGCCAGAGGTGTATGTACAAAATCTAAATCCAGGAAGT
ACACTTTCTTCACCGGCTTGAATAGCAAAAATTTGTGGTATTTTATGCTTAGGATTTAATGTAACAACCG
GCGCCGAAGCGCCGTCAAATAATTCTGTAATAAGTTCCATGATTTATCCTTGAACGAACTTGTAAGGCAT
GTTTGCAATATCTATGCAAGACGCAATAATTCCAAGAGATGATTCTACTTTCTGTTTAACATTTGATGAA
ACAAATGATGCAAAGTCAACTCTATCTTCTATATCACCTGTCATTGTAACCAATTCACCAGTTTCCATTA
AATGGTCTCCGTCATAGATTACCGATTCGGTGAGTTCTTCTGTGGTCATAACTTCAGCTTGAATAAGCTT
GTTGTTAGTTTTAACTGTTCCATCATTGTAAGATGCATCGGTTATTTTATTAACTTTGACCATTAATCCA
CGTGGCAAAATGACTTCCATTTCATTTGAAGGCGCTAAACTTCCACCGGGTAAAACAACATTGACCTTAT
CAGCCCCAGTAATAACCCATCCAATTCCAACTAAATTATCGCTAGAATTTACAAGTCCTTCATCAGTTTT
ATCAATAGAAACGCTTAAACGCTTTTCGTCTGGTAAAACACCTATAGATGAATCAGTCATCCAAGTACCA
AAAATATTTGGATATAATGATGTTGACACAAAGTTTCTAAAATAAAAAACTCGATTTTTTACCATTGCTT
CGTATATTGAAGGTAACATTCGTTGTGAACGATACAAAGTAATACCTTTTGGTAATCGTTCACCATTTTT
AAAGGCTGAATCTAAATTATCAATAGCTTTTTCTATGTCAGATGCTGTCAAAATACTTGTACGCTCATCT
GGATTATATAATCCCAAAAGAGCATTATTTATGTCTACATATCCTGAACCTACGTATTCACGAATTCCGC
GTTTTTGCGCTGGTGTGTATTTAGATGAATCTTTATTTTCGACTATAGGATGTAATGACCATCCAGCGGT
AAGAGCATATCCACGTAATTCTCTTCGAATAATCTTTGTTTTTATTGCATTCCAAGAATTTTGTACTAAC
TCATTTGCAGCATCTTGGTTTAAATGCGAATGTTTGTTAATATTTCTTTCAAACCAAGCACCCTTATATT
TTTCTAAAGTATCATCGACGATAGAAGCAATAGTTTCTAAGGTTTTTATTGAAGTAATAGATTCTTTTCG
TAATCTTTCTAAAGCTTTATTTTTTATTTCTGCGTTAATAATGGATTCTTTATCTTCAGGAATTATAGAA
GCTTCTCTGAATGCCATCCCAGCTGTAACACTGCTAAGAGCATTCTCTAGTGCAAATCCTGAAGTAGAAA
TTACCGTTAATTCATTAGAATCAGAAATTAAAGGCGCGTCCGCTGGTTTATTTAATTCGGCCGCTGAAGC
CTCAAATTTTTGAAACATTGGTGTTTCAAATCTAGAAGATTCCAATGACTGACTTTGCGCAATTGCTCTA
CGGGAAATTTTAACTTTAACGATTACAGCTTGGTCAGAACGTTTATCATTTTCTTGCGCAATAGATGCTG
CAATTGCCTCATTTTTAGTTACTTGAGCCCCAGTATCTTTATTGATATAAACATCACCGACCTTCGATTC
AACTTTAGTAAAGAGCTCGGTACTAATTTCCGGAACTCCTGGAATGTCTTCTAGTGATACATTTTTGCGA
TGTATAAGAATATATGCATACTTTTTATCGTAATCCCAGAGTTCCTTAAGAAGGACGTATCTACCACCTG
AACGACTACGGATAAGTCTATCAGCAATAACTTGAATTTGTCGAGCTTGGCCAGCAGTTTTAGACTTAAG
AATACGGAGCATACAGGCATCAATTTTATACTGGCGCATTGTTTGCATTGCAACAGTAAAAACTGAATTG
ATATAATTAATTGGGCTTGGACCAAGACCTTTTAATTTAGCAATTGAACCTTTAGCAGTTAATGTAAAAG
GAACAATATGCATCATTTTATCGCCCATCTTTAAATCACGATTAGTATCACCTCCAGATGTATAGGTACA
TAAACGAAAGCCTGGTTGTTCAATTGCATCATCAACATGAACTGAAAAAATTTGCGGTATTTTCTTCTTT
GGATATAAGTTTGTAATTGGAAGAGTAGTATCTTCGTCAAATAATTCTGTAATAAGTTCCATCATATCCT
CTCTAGTGTTTATTCTATTCTATTTATAAAATTAAAGGCCCGAAGGCCTTTAATAATCTATTGGTAAGAG
AGTACGATATATTTCAAACTTTGGACCTTTTTCATAAGCATCAAATGTTTCTGTGAATTTATTATAAGCA
TATGCATCTATAAATTCAATCATGATTTGTGATACAGAAGTAGAAACATCTCCACCTTCTTTTTGAGCCA
CGACAATTGTTTCTAAGTAAGCTTTCATAGACCAGTTACCTCATGAAAATCACCAAATACATCTTCGAAT
GTATTAGCTTTAGTTTTATCTTCACGTAAACGAATCGCAATCGGAAGAAATAATTTAACGTAATCAGTGC
GGCCATCAGATTTTAACCAACCGTTGCATTCGCACTCTAGAATTTTTCCAATATAATAATTTTGGTTTTC
CATAATGCGAGTACGGTCAAGTTCATGCGATTTTACACCGGCTTTATCTTTTAAGCCTGAACCAGCATTT
ACCTTAATTTTTCCACACTCTGACTCAAGAATAAATCCACCCGCTTTAGTAGGGTCTTTACGGTGAGGAT
AAATTCCTACAATTTTTAAATCAACATCAATTACTTCTTTAAATTTATAAAGATTTTTTGAACGAGCATT
TTCCCATAATCCATCGATATTTTTGAGAATAATACCTTCAAGACCTTGGTCAATATACTTTTTATAAATT
ACCTTAGCTTCATCTAGGTTATTTACTACCTGGTTTTCAATTAAAATTACTTTATCATATCCAGATGTCA
TTTGTTCTAGTTTAGAAAAACGTACATCATATTTCAAACGAAATGCAGGAAGACTGTATATTTCTACCAA
CGGGACATAATCCCAGACCTGAAACTTCATGCATTGTGCTTCTTTTTCAGAAATGGTTCCCTTTAAAGAT
TTATTGGCGATTCCATTAGAAGCAGTACGTGATTCAGCTACTTCGGCGAATTCTTTAGCTTTACTGTTTT
CAGGATAAGCATCAAAAAGAAAATCTAGGCCTTCTGGCTCCTTTTTAACTTGCTCATGGTATACCAATTC
GCCATCAATCAACACACCTTCTGGATGAATCTGGGGGGCTTCAGCGGTCATTTTAATTAACTCTTCCTTA
AGAAGATCTAATCCTAGATATTCATTACCAGCTCGTGATAAAAGACGAACATCATCTAATTCATCACCTC
TAACTTCAGCAAAACACCGAGCTCCATCAGCTTTTAACTGAGCAAAGGCTGGAAATTTGATATTCTTATT
AATGCCTTTTTCATCATAAGAACTTGCGAGCATTTGAGGTTGTTCAGGAATTAAACCTGGCCAAACTTTG
TTTGCAATAGATACTGAAGCACCACATTCAAGGTCTCGCATCATCACTCGACGCAAAACTTCAACATCAT
CTTTTTTACCATCGGTGATATATCCAGTTAATTCCTCAATTGCTGCATTTCCAGTCAATTTCCGAGTAGC
TAATGTGAATTCAATGAAGTCAAGCATATCGGTAAGAGTCAACATTCCAAAACTCTGGGTAGCAATACCA
GGTTTAGGCCATTTCTTGATATAATACTGTAACCCACGAGAATAAGTCAGACGATATACTCGTTTAAGCA
ATTCATTATCTTTATTCTTTTCAAGAATTGCTTGCTTCTGTTTAGTTGAACCAATAGATGCTATTTCGTT
CAGAATTTTAAGAATCATTGTTCATCCTTTAGAGTTTGGTTTACAGCTCTATTATAAATCAATTCATCAT
TAAGCTCAGTCAAAGACCTGTGGTACGTGGTTCTAACTTTATTTCCTTGCATCCAGTGCTTGATATAAAT
GAAACCTTGCTCTACACATTTTTTAAAAATTCGTTCGTCTTTTTGAGCTCGGAATTCTGGATTGCATCTA
AAAAATTGATTTACGTGACCGTAATCACGTGTAGTATTACCTTCATTTTCATAAATAGTGTGAACAACAA
ACATTAGAATGCTCCTTGGAAAATATTATCACCACAAGTAGGTCTATTATACAAATACTCTATACCGCCG
GGCTTAATATAGTTCCATGTCTGAAACGGATGCGTCTGATATGGATGATATGGATTATAAGGATTAAATC
CAGGAGTTCTCCAGGTAGTGTTCCAAGGAAAAGAATCGTTTTTAATCATCTTTTCAATTACATCTTTTAT
AGCTTTTTCACTATCAAAACTTTCTTTATTTTCCTTTGGTGAAAAAAGCTTATTCTCTACATCGTTCCAT
GTATAAACTCGCTGAGCAGTTTTTGGAATATTGTCACGCTCTCCTCGAGCCATCCAATACACAGGAACAC
GTAATATTTCACTCGCGTGATCGCAGTGGTGAGCGAGATCGTCAATATAACAAATTACGTTATATTTCTC
TTTTGCTTTTTTGAACAACTCTTCTTTTGAAGAATCATGACTACACATCAGTACTTCTGAGAAAGCACCA
GGAAAAAGAGCATTCAAATTAAATTGACGATTTAATAGAGCGTCAATAGAATCACCCAATGCTGTAACAG
CAACAAAATTATAATCTTCTTTTAATTTGTTAATTACACACAGAGCATCTTTATATGGAGACAAGTAACG
AATAAAATCCGAACGATTGTATTTTTCAATTAACTTGACGCCAAGTTCTTCATCACAGTTAAAGAGTTTA
CCAGGAGAAATAAATTTCTCATCTTGGATCATTTTTAAAATATGTTCTAACGGAAGATTATATTTCTGAG
CAAAATAAGGAAGGCCTGATTGCCAGCTTAAACATACTCCATCAATATCAGTTAAAATAGTAGGCTTCAT
AGAGAGTCTCTTAATAGGTTTAACACATCAATAAATTCAGCTTCGGTTAGTATTGTATCATCTTTTGTTA
GACCACTAGCAATGCTGTGCTTCAAAACTTTTCCTTTCGAGGCTTGTAATGCATCACGAAAGCCCTTGTT
TTGAATCGCTGCTTCAAAATATGCATTTGTGTATAATTCTTTCCACGCCGGGGAGTATCTTGAAAACGGA
ACTCCAAGCCAAAAGAGGGTCCCACGGTCCTGAGCTCTAGCATAAGACCTTCCAGCTTGTTGGGCGGCAA
GCCCGGATAACCCAAATATACGACGTTGTTGTTCAACATTTTTCACCTTACACCCTTGGAGGAATCCTTC
AAGACCTCCAAATTGAATACCATCCATAACGAAAGGCCATTGGGCGAAATTACTTAATGCACATGATGGC
CACCTAAAATTGCTTCTAATCTCTAACTCAGACATTTTCAATGCTTATAATTTCAACATCAGCCCAATGA
CCATAGCAAGGAAGACGAAATTCAACTGGCCAGTTAGGGTCCCTTTCTAATATAATAGACTCAACTTGTG
GTTCTTCGTGTTCATCAGTATACGGATTTTTATCTGTTACTTTATATGTGACCTTAATGTACTGAATTCC
AAAAATCTTATTAATTATATTCATACTAATTCCTTTAATCCGTAGATAGGAGATAATTCATCACCCATAC
GAAGGTCTTCATTTCCATCTACCCAGGAAACAATATAAGCCTCTTTTATTTGAATACCACTCCATTTAAA
TGGAGGTAGAACCTTAGAAATTAATCCAGGTATACCAACTCCCTTTAATTCAACAGTTTGACCTAAAAAG
AATTTCATTAGAACCTCATCTGAAAACCGTGCGATTTAACATTACCGCCGCCAATATCAGAAATGTTAAT
TTCACGCGCAATTGAAGGGTCAATGTCAATTTCACGATTGAGTTTAGTGATAGCTAAAGTATCACGCCCA
TTTACAGTACGGAACTCTAAAGGACAAACCACATCAACGTAGTTTTTAATCGATTCGCTAGTAAGTTGCA
AATGTGCAGGAAGGTCTTTAGGTGCCTTTGAGAAAACCACTTCACAGAAATTTTTGCGGGTATCAAAATA
AGTAGTCATAAACATAATATTTTCCTCAGTAAGGGGCTGAAGCCCCTCATTTTATTTTAAATATCAAATT
CATTAAGAACTACATCAAAGATTGCTTCAAGATGCTCAGGTTTAGCTCTGTTACTCAGAATATGACGAAT
CCAAGTTTTAACTAAGAGTTTACGATTAGCACCATTCCAGCAAGGATGAGTCCCTAAATCGCGTTGGCGG
AAATCATCATCCAGAGCGATTTTGAAGTTGGAACCTTTCATCGTGATTGAAACCGTGATACCGTTTTCAA
ATCGCATATAAACGTAGTTAGGAGTCATATACTGTTCAATTTCGCATACTGATCCATTTTGATGTTTCCA
GAGGCAAATAGTATCAATAGAACCTGCAATACCATTAGAAACATATTTACGTTCAAAGTTAATGTAGTTC
ATTTTTATTCTCCGAGATGTTTAATTGCGGTACAGGTATATAATATCATATCCTGTACCAAAGTAAACAA
TTATTTTACTACTTTCCAATGCTGCATGTCAAGTTTACCAACTTTTTTCATCTTCTCAATTAAGCGTTCT
GCACGTTGGCGAGCTGTAACATAATGCCATTCGCCTAATTCATTTTGTTCAATTTTTCCAACGATTACTG
TATTCAATTCATAAATCCAACCAGTAAAGAAATTATGAACTTGAATTGTAAAGGTGAAATCTGTTCCCAT
ACCTTCTGTTGTTTCTACTTCAATAATATCACCTTCAACTGCCATTAAGAACCACATAGTTTCATCATAT
TTACCATTGAAGCATTTAGTTTTAACTGCAGCGTTCAGATTAATCGTTTTCATTTTATTCTCCTTTGTTT
GTGTAAGATAATACTATCACAAAGGAACTATACTGTAAACAACTTTGTGCAATCTTTGGAAAATAAAAAA
GGACTCCCGAAGGAGTCCTCAACTTATGCTTTCTGCTTACCAAAACGAGAAGCATCATCTCGAAGAACCG
CACGTGCTCGGCGCATGATCTTCTCAACAGTTTGATTGATACGAGAGTTCGACCCACGCTTGTAGCCAGC
GCGTTTAGAATCACCAACTTTCTTTTCAACTGCTTTCTTTGCTTTAGCTTGTTTTGCCATTATAAATTCT
CTTTTAAATGAAAATGCAGGACTTATTGGCATTGCCTGCGCAAGCCCTCAAGGGGAACATAGGTTTTTGG
ATATTTAACGACCAGGATAACCATAAACCCGTCATCATTCACATTCAAGAGGTACACCGTAAAACTGTCG
GGGTCTTAAAACTATAATGATTCGCAAATCATTAATCAGACAGTTCGACGGCTCCTCGATTTAGCTCACA
CTAAGGCAGTGAATCTCCAATAAATTACTTCAGTGTTACCACAAAGTGACGAACTGCTTTTCGTGCAGCA
GAAGCCAGAGGCTTAGCATATTTAAGTTCATCTTTTTCCTGAAGCTCAGCAGCTAATGCAGTTTGAGCAG
GATTCAGATGTTTGAAATAACGCAGGATTTCAAGAGCTTCGGCTTCAACATCAATAGATGCGCCATAGTT
TTCGTGACCATTATTCCATGCGTTTCGTTGCAGTTCAAGAGCGTGTTGTAATTGTTTAATCATTTAAAAA
TTCTCGTTAGAGATTAAAACTCGGTAGTCACGTTCTTCTGAATTTCATCTTCTTTCGACAGATCTCTCAG
TTGTAGACTACCACATAGAATTGTTCGGTTAACTTATTATTCCGACACCCAATTCATATTATTATTTATA
TCACTTATAAAGACACGGAATAGCTTTATAGTGACAGGTAACGAATTTTTGTTTAATTTCTTTTGGCTGC
TTAAGACCCAGAGCTACAAAAGGATGCGGAACATTTCGAATTTGACCAACTGGAAGAGAAGTCAAATCAC
CAACTTCGCAGAAACCTTCAGGAACATCAGGACCGACAGAGTGAACTACACACAGTTCAGGAACTTCACC
TTGAACACGTTTACCGATAATAAGTCCTGATTCTGTAACTTCTTCATCACCGGCTTGTGCAGGTTCAGAA
ACTAAAATAACATATTCACCGACAGCACGAATTGGTAGCTGTTGTACTTCAGACATCGTTTTTCCTTTTT
GTTAACAGATGAATTAATAATAACAAATAGTTCTTAAAGCATTTATTTACCAATAAATTGAAGCAAATGC
TCAACTTTCATACCATTAACGGAAATCAATTTGTCAATAGAAAAACCTCGCCACGCACCAAGCTCAACAT
CAAATACTGGAATCATGTCAGTAGATTCTTTCCGAGTAGATTCAGTCAATTTGCCAGTTTGCATGGTTGG
CATAAAGTCTGCATCACGAGTACCTTTCATAGTACGAATAGTACCATCAGACTTTTCAAAAACTACGTTT
GAAACACCCATGGACAATTTAGTTTTCAAAATTTCACGAATTGCTACTTTCTGCTCAGTTGTCAGTTTCA
TTTATTTACCTATTACAGTTTTAATATGAGTTGTTCCACGTTCTTTAAGGGTGGAAAGTAATTTTTGGCA
TTTTTCTAAATCAGATTTCCAACTATATGGTCTATCAATACAAACCCAATTTGTCTTATAATACTGTTTC
CATTTAGAGAAAAAATATTTCTTATACTCTACAGCAAATGAGATGTTCTCGTTAGAATAAGAACTAATTG
CTGTGAGTTTTACCAAACGAAATTTCATTATTCACCACAGAATTCGTTGATATTTTCCCAGTTTAACTTA
TTCAAGTTTTTCTTAGGAACATTAAACACTTCAATACCTGCATTTCGCAGAATATCATCCCAACCGGGTT
TATTTTTGTCGTATGTTTCACAATAAACCAGCTTTTTAATACCAGATTGAGCTATCGCTTTTGCGCAATC
TGGACAAGGAGAAAGTGTTACATACATAGTAGCACCTTCAATAGAAGAACCATTTCGTGCAGCAAACAAA
ATTGCATTTAGTTCAGCATGAATTTCATTTTTAGATGACCATTCCGAGTGAGCACTACGATGTTCTTTCG
CCAAAACAAAACGATCAGTTGAACCAAATGATACGCATTCAGGCTTATGACCTTGAATGATAGCATGTTT
AGGCTTATTCAACAACCATCCTTGCTCAGCAGCATAATCACAACAGTTCACACCCCCTGCGGGTGAACCA
TTATACCCAGTAGAAATAATACGTCCATTCTTTTCAATTACTGCTCCTACCTTCCAGGAGCAACATTTTG
ATTCCTGCGATACTAAATATGCAATTTGAAGTACTGTACTCGCTTTCATTTCATAATCACCAGATAAGCA
GATTTAGCAGTTTCAACACGATAAATTTCGTGACGAAGTTTAGTTATACTTTTAATAACAGAACTAATTA
TATTCTGCCCATCTTTAAAGCGGTTTTTCTTATCAATAAAAACTGCGCCAGTCATCTTTTTGTGAAGCTC
AACTGGATACTTCGTCACAATAATAGCATCATACACAGAAGGATGAATACTATTCACCAGAGTATCATTC
ATTAAAGTTATTCTAATGAACTGTGCTGTTTCAGAATCAAGCGCTCTATGATCGCCAGTATCATTTTCAA
GACAATTATCAATTATATCAGTTAAATTCATCATAGTACGCCATACACCCTTTGTGCTTCAACTAATCCA
TCAAAATCCAGTTTAAGATGCGATATTTGATCGCCATCACCTGGATTCACAATTACTAATACTGAACGAG
GAGTTTCGGTAATAACACGAACCGATGTTTCAGGAAATCGTTCAGAAACCTTATTTACTAATTCCTGCGC
AAATAGTTTAACTTTTTCTTGGAATTCCTTTAACAGTAATCGGTTTTTCACTTAGCAACATTTTGTTTTC
CTCATTTGTTTTGGTAGAGCTATAATATCACAACTCTACCGTAAAGTAAACCATTAAATCGCTTTGAATT
CCGCAGTTTGAGATTCAAAGCGAATATCGCCTTTGATAACAAGCTCAGCATCAAGACCAAATACGACAAT
GATATGCGCGGAACCTGGATACAGTGTAATGGCAATAGAATCCACCTGGTCTGGAAGCAAAGTGTTCAAT
ACATGAGTCACTTGAGCATGGATTCGAAGCTCAGCTGCGTTATCAAGTTTTTCAAACATATTATTAGCGA
TAATTTGGCTAAACACTACTTCTACGATTTTAGAGTAAGTCGGAAACATATTTACCTCACATAATTTTCT
TCAAGCCAATCAATAACATCCAACGCATTATCAAAAGTTGAACCATCTACTCTGTCTTCTGTTTCATAAT
CAAGAACATCTAGGCCTACTCTTCCGTCAACAATAGGCCATAGACAAAATAGATATTTCTTTTCTTTTTC
AATTTTATCACAAAGACGATAAATCTTTTCTAGGTTATTCATAAGTTTTCCATGGTAAAGGCAGTTTAGT
TTTCTTTACTACTAGTTCAACATCGGGATTCTTTTCTCTTAATTTAAGACATTCCTCCCATGCTCTATTT
TCACTAGTAAATACACAAAATTGCCCATTACTAGTACCAACTAAACCGCTATTTACAATAACAATAGCCC
AAGTTTCATGGTGCCAAGCCATTAAAAATCTCCCGAAGCGACTTGCCAGCATTCAACACCGATACGACGC
CACATTTCAACTACTTGAGTTCGGTCATCAATAGCTAATTTCACGTCAAAATGCGGTGCAATGTGTTTCC
AGAAAATTTCTTCTTTAACTACATCGTCTTTACGGGTATCGCCTTGTTCGCGCTGACATTGCATAACTAA
TGGAACGCCAGCAATGTCCTCAACCCATTTACGGGTCATACGATAATATTTCGTTGGGTCTTCTTTAGTT
CCACTTTCACGACCTGAAACGACTACGATTTGATAACCCATAAGAGCATACATCTTAGACAGTTCAACAA
CCATAGGATTGATAACATCGGTATCGCATTTTTCAAGGTCATAAGGACCACGACCATTCATTTTAGCTAG
TGTACCATCAACATCAAAAATAACTGCTTTTGGTTTACCAGGAGTCCCATTATATACTGGAAGACCGAGA
TACTCTCGCATGCTTTTATACATTGAACGTAAAACATCAATTGGTACTGCTTTAGTTCCGCGTTTTGAGT
TACGTTTAACCAATTCAGTCCAAGGAACATCAAACACTTTATGTTCAACTTTCCAGCCGTATTCTTTGGC
AAAAGTTTCCCATGCTAGGCGACGTTCAGGATTCAGGTTAGTATCTGAAATGATTACTCCCTTAACAGAA
TCGCCACCGTACAGAATACTTTTAGCTGTATCAAACTGCATACCAGTTACGATACCTTCTTTCTTTTTGG
TATACTTGTACTCATCGCGTTCTTCATGCGCCATAATAGATTGGCGATAGTCATCACGATTGATATTATA
AAACCCGGGATTCTTAGCAATAAATTCACGAGCCCAAGTACTCTTACCAGAACCAGGACAGCCAATAGTC
AAAATAATCTTTTTCATTTATTTTTTCTCAACTAATGATTGAATATAATCATGTAGGTCTTTAGATGCTT
TACCCCACTTATTTTGATATTCATTTTTGAGATTAGCACGTGATTGAGCTAATAAAACATCATTAGTTGG
AGGTAAAGATTCTAACCGCTGAATCTGGCGTCCATAAATCATTGCAGCCATCTCGGATTCATAAATCAAT
CCTTTGAGATGTTCAAATTGATGCCATGAAATCATTTACATTTATCCTCTTTTAGCTCTTGACGATAATA
ACATRTCATAGTTTTCTGGTCATGTACATATCGTTTTACATCATTAAGCCAAATACGAAATTCCTGAGAA
TCTTCAAATGGCATACCGACCCAGGCTTTACCATCAATAACTTTAACTTGCCAAGATAGTTTAGCTTCAT
CATATGACTTTATTTGCACAGGCCAATTAGGATGAACTGTTTCTTTCTTTACTTCTAGAGGCTTTGTCGA
ACAACCAACTAGAAGACCAATAGATAATATTACTGCTGATAGTTTAATCATTTAGAAAGGTCCTGGATGT
CTTCTGCGAACTTGTTGAAGGAGTTGTTGATTTGTTTTTCAACCAATCCTGGCTTATGAGCCACCACATC
CGCCTTCTTTGCATCTTTGCGCAGTTTTTCATTTTCACGCTCAATAGCAGCAATTGCCTCACGATTTTTA
TTATTCATCGCATCAATATAATTATACTGAATTCGCAAATTATTTAATGCTAAGGCGTTTTCATTGGCCG
TTTTTGTAATTTCAGTAACTGATGTTTCTAATCTTTCTACCTTATGTTTTAAAATAATAGATGTTCCGCC
AAATGCTATTACAAGTAATAGCAATCCAGCTGTAAAATTACTTACATGCATAAAGTTTTAATAACCTCTA
CAATATCGTCTTGAGAAAGACCGTTAATTAAAATATGATGTTCAGCTGGAGATTTAGAAATTTTAAAGCA
TGCCTCAACATCTTCTGCCATATCCGATGCACTACGATTTGGATTACTAATACCAAGGCGATGTTTTCCC
GTCAAAGGATTAACGATGATATAGCATTTGCAGCTATTGATATGAACATTGGGTTGAGTCTGATTAATGA
ACACTTCACAATCATACTTAGCAAGTTGATTTTCTAAAAAGACTTTCATCTCTTCAACCGCATCAGGAAG
CATATCACGGGCTTGCTCAAGACGACGATTTCGATATTCTTTAATGGTCGTTTTCCGCTTGACTTGCTTA
GCTAAATCTTTCTTAAGACCAGTGATATATCCAACTCGACGATTTCCTTTGAATACAGAAATCCCATCTG
TAGTATCACCGTATGCTTCAACGACCATTTCAGTAGTAATAAGTTGTAAATCCATCATAAAGTCCTCATG
TTATGTCAGTAAGACTACTATAACACAACACGAGGGACTTGTAAACAACTTAGTATCCTTCTGGGATAAA
TTTTTTATAATTTTTCAAAAAATTCTGTTCGATTTCACACATGACCTTTTCTTGACTATCGTACCCCTGG
TATAAGCTCATGATGATACCGAACAGGTGATCCATTCCAGCACCTTTAGCAACACCTTGTGCTTCCATTG
CATAAGTCTTTCTATCCTTACCGCAATGCTTATTATGACAGTCAAGAACTAAAAACAGAGCTCGGTCTAA
GTACTTCAGATAAGTCGTTTCAAATGCTTCAATTTTTCTGTATGAATATTCATCGTCAGCATACATTGCT
TTAAGATCATCTGATGCACCATCAATAATAGTCTTAAACAATTTTTCTGGATTATCTAATGAACTTTTTG
TACTATGAAGAGACACGTACCAGTCAGACTTAATTTTAAAATGAGAACCATCTTTCATCACAGCAACATA
GCCTTCGATGTTTTCTGCATTTTTAGCTTCTTCTATCCATTTAGGGCTATCGATTTCGTATCGTTCAACT
AGATACGGACGAAGAGTAGCATCTTTATAAATATCATCGTATGAAATGTATTCACCCGTTTCGTTTTCAC
GAACATTCAGTAAAATAATTTTCATCTCTTGATAAGCAAGAACGATTCTATTCGTCGGGGCAACGAATTC
GAAGTTAGCAGTAAATCCATCTTCAGCTAATTCTTTAAGTCTATCACGCAACCGATGGTGATTAATATTC
ATCAAAATTCCATTAGCCATTAAAGCCTGCTCAGATTTGATTGAACCCTTTGATTTGAACAGAATTTCAT
CACCGTCTAAATAAGTTGATACCAAAGACCCGTCTTCTTTTGTTAGAATATAATCAACATCGTTTAAATC
GATATTCATCGTGAACGGATTTTCATTCAAGTTAAAAAACTTTTCCATAGGACGAGAAGCAATTCTTACT
GGTTTTTCTCCATCCATTTCAAACATAATTCCACGACATTCTAGTGCATCTGGAAGTAACCAATCAGAAT
AAGATGCATAATTATATGAGAAAATTCTGTAAGTTCTTCCAGATGCACTTACATCATCTGAGTAAAAAAA
CTTACGCTGCGAATCCTTACATAGTTCCATTAAATTGTTAAAAAGTTCTTGCATTGTGTATCCTCTTTTG
TGTTTTGAATATAGTACCACACTCCATGTGGAAGCATCATTTTTTCTTGTGTTGAATATTCCAAGGCGGG
TTAAACAGTTTAATGAATAGAGGCTCCTCTAAGTCAATCGTTGCGATTGTCATTGTACCTAACTCATTTG
TCATAGAAAGATTAAAACATTGGGGGGCGTAAAATTCAACTTTGCTTCCTTCCTTTAGCGCAGAATGAAT
TAATGCAGATTTAGTAGAATCAGACGTTTTGTCTTTGCGGTTAATAGCAGTTCTATAATAGTTTATTCTT
TTACGTAAATTTTTAGTTTTTCCAATATAAACAAGCTCATCATTTATAGCAATAGCATAAATTACGTTAT
ACTTGTTTGGAATAGATAATTGTTTTATACTTCCATTGTCGTCTAATTCTAGCTCAGTATATTTAATAAA
TGAATATTCTGTTGCAATTTCTTTCATAATAAAATGGGCCTTGCGGCCCACTCCTTAAAAGTATTTTTTA
AAACTCATCATAACTTTATCATCAACATCATTATCAATCTGTGCAACAAGGTAAGATGACAGTTCTACTT
CTTGCGGCGCGGATTGAACATTATCAGAATTAAGATATTCACGAATCCAAGGATATGGATGTTTAACCGG
AGCATCGGTAATTGGGCATGGAAGACCGCACTGTTTCATACGAGATACAGTTAAGTAATCAATAAAGCTC
CACATGCTATTTGTATTTAATCCAGGAACATCACCATCTTTAAATAAATGAACTGCCCAGTCTTTTTCCT
GGCGGTTAACTTCCATGAAAATATCAACTGCTTCTTGTTCACACTCTTGGGCAATTTTAACCCATTCATC
ACCATCAGTACCGGATTGAAGTTGACGAATAATATATTGGGTGCCTTTAAGGTGAAGCTGTTCATCACGT
GCAATGAACTTCATAATCTTGGCATTACCTTCCATGATTTCCATGTTCTTATGGAAGTTAAAGGTACCGT
ATGTTCGATTGAGCTCGCTAATTCTCAACCCGTTCTCTTATGAACTGCTGCATGTTACCATGCAGTCCAG
ACTATATCACAATCCCGGAGGGATTCTCCCCATTTCGAGTCGCTTGACCCTACGTCCGAAGACTAGTCGT
TGAACCTTGTTTTGCAATTGTCGCCGTGCCATCGTTTATATGTTGATGGTGATAAATCTTTCTTATCACA
ATATGGACAATTCAATTGAATTCTATTTGGATTTAATTTACATCTGTCATTATGTTTTAGATAAAATCCA
GGACCTTTACCAATGTGACCACAGAAATCACATGTGATTTCTTTTTGTGCAGGATGGGTCCCGTTCTTAA
CCATTTCTAATGTTTTTGCTGATGTTCTTTTCTTATGTTCTTCCATCAGAATCTTTAGGTATAGGTCCGT
TAGCATCTATCCAAATTTTTCTATAGTTCAATTGTGCATCCTCTTAAAAGTATAATCATATTTATATTAT
ACTAATTAAAGGTGCAAGCAAAACCTTGGCTGCTAGTTTTCCATAAAGGACTTTCCAGCAATTAAAGGAG
TTTTCGATAAACGTTACCGTTTAAAGGCGCATTTTACGCAAAAGATACATAAAAACGAATAGCTTCTAAG
GCATTAATAACGTGCAAACAGAGGTAAAGAGATTTCATCAGATCTCGTTTAGCTCTTTGCTCAACGTCTT
TATCAGCTAGAATTAGACCTTGGTCTTTATAATATTCAACCATGTCTTTAGCATTTTCCCATTCACGGGT
TTTAACCAAGACATCATCGTAATATCGCCCAATGGACTCAGCACGTTTCATAATAGCTTCATCTAATACA
ATCTCATCAAACACCTTCGATGGATCAGTATAAAGATTTCGCATGATATGAGTATATGAACGACTGTGAA
TAGTTTCACTAAAAGTCCATGTAGCAACCCATGTATCAAGGCTTGGGTCTGAAATTAATGACATAAGTAC
AGCAGATGGCGCACGACCCTGAATGCTATCCAAAAGTGATTGATACTTCAGGTTGTTAGTAAAAATATTT
TGCTGATACTGAGGAAGCTTATTAAATTGCGCAGCATCCATCATTAAGTTTACTTCTTCAGGACGCCAAA
AAAAACTGATCTGCCGCTCAATGAGTTCTTCAAATACTCGATGTCGTTGAATATCATATCGAGCTAAACC
TAATCCAGAACCAAAGAACATCGGTTCATTCAAAACATCAACTGGATTTGTATTAAAAACTGTACTCATT
TAGAATCCTTAAATTTACATTTATCATAATGCCATCTTAAAGCATTGCCTTTATTAACTTTCTTACCACA
GTGCGGGCAAGGTGGATACTCTGCTCCTTGTTTAGTTCTCAAAGAAATCAAATCACGGGTTTCTTTAGTA
TGAGGAACATCTCTTGTCGGAGAAATTGTACCATACATCGGATTAAGCACGCCGACTTTAGCAGCAGCTA
TATTCTTTTTAGCTTCTTCAGTTTTAGGCTTTCTCATCTTTTTCTTAGGTTTCTTCGCTTTTAGCCCTTC
CTTTGGTTGCCGCCGATATTTTGTGTTTGGTTTCTTCTGTTAAACGCAGACCTGTTTTCGCTTTTTGACA
TTTTCAGTCTCGTTTCTTCAGAAATGACCGTACCTTTGCGAAATTGCGAATTTAACTTCTTTGCATGGGC
ATATATTTTAGAATGAACTTTATAATGACGTTTCTTAGTTCCTTTCATATTGCACATCATGAAAAATGCG
AATATAACAGATTTTACCGGATAAATCTTTGATAAAATAGCATGCGCTATAAAATGCTCTCTAGCTGTTA
ATTCAACTAAATTTTCTTTATCATCAGAACCTCCCATGCATCTAGGGATTATATGATGTGTCTCTTTATA
TTCGGATAAAGGTTCCCGAGCCTGAGCTCGGGAAATTAGGTCGTTATAGATTTTTTGATAATTCATTACA
ATTTACACGCTGCACAATCATCGGCTTTAGGAGTTTCTATTTCATAATCATCAGTACCAGAACCATCACG
GGTATTATGATAATAGAAATTTTTAATGCCATAATACCATCCGTATAGCATGTCATCAATCATTATTGAC
ATTGGAACCTTTCCTTTTGGAAAAATCTGCGGGTCATAATATGTATTCGCTGAAGCTGATTGACATACCC
ATTTCAGCATAATAGCTACCTGCGTAAGATAAGGTTTATTACCTTTCTTAGCTAATTTCCATGTATAATC
ATAGAGGTCTATGTTATGCTCAATATTGGGCACGACTTGATTAAAGGAACCCTCTTTTGATTCTTTAACA
CTTACCGGTCCACGTGGAGGCTCGTAGCCGTTTGTACTGTTAGAAACTTGGGAAGATGACTCACATGGCA
TAAGTGCTGATAATGTGCTATTACGGATGCCAAATAGCTTAAGGTCTTCCCGCAGCGCCGACCAGTCACA
AACGTATTTTGGAGCTGCGATTTGGTCAATCTTTTTATTGTACCAGTCGATAGGTAATTCGCCTCGAGAC
CAACGAGTGTCTGAATAATATTCCGAAGGTCCTTTTTCTTTGGCGAGCTTAATGGATGCTTTAATGAGTC
CATACTGTAATCTCTCAAATAGTTCATGTGTTAAATCGTTAGCATCTTCATAAGAAGCAAAGTTACTTGC
CAGCCAAGCTGCATAGTTGGTAACACCTACACCGAGGTTACGACGCTTTTTAGCTTTTTCTGCTTCAGGA
ACCGGATATCCTTGGTAATCCAACAGATTATCAAGAGCACGAACTTGAACTTCTGCCAATTCATTAATTT
TATCTTGGTCTTGCCAGTCAAAATTATCTAATACGAATGCAGAGAGAGTACACAATCCAATTTCAGCATC
AGGGCTATTCACATCATTTGTTGGAATAGCAATTTCACAGCACAAGTTACTCTGACGAATAGGTGCCTTT
TCACGAATAAACGGAGTATAGTTATTCGTATTATCAATGAACTGCACATAAATCCTTGCTGTTCCTGAAC
GTTCAGTCATGAGCAATTCAAATAGTTCACGGGCTTTAATACGCTTTTTACGAATATTAGGGTCTTTTTC
TGCTGCTTCGTATAATTCACGGAAACGGTCTTGGTCTTTAAAATAAGAATAATAAAGCTCGCCACCCATT
TCATGCGGACTGAACAAAGTAATGTAATCGTTTTTTCCAAAACGTTCCATCATCAAATCATTCAGTTGAA
CACCATAATCCATATGACGAATGCGGTTTTCTTCTACGCCTTTGTTATTTTTCAAAACGAGAAGATTTTC
AACTTCCAAATGCCAAATAGGATAATAAGCAGTAGCAGCGCCGCCACGAATTCCACCCTGTGAACATGAT
TTAACTGCAGTCTGAAAATGTTTCCAAAAAGGAATAACACCAGTATGGCGTACTTCACCCATGCCAATCT
TAGAACCTTCGGCACGAATCATACCAACGTTAATACCAATTCCAGCGCGTTTAGAGATATATTCAACAAT
TGAAGCAGAAGCCTTATTGATAGACTTCAATGAATCTCCTGCCTCAATAACAACACATGAACTAAACTGT
CGAGTCGGAGTACGACAACCAGCCATAATAGGAGTTGGCAATGAAATCTGTCGAGTTGATACTGCTTCAT
AAAAACGGATAACATGTTTTAATCTATCAACAGGTTCATCTTGATGCAGTGCCATTCCAATAGTCATAAA
TGCAAACTGTGGAGTTTCATAAATTTGACCAGTGGTTTTATCTTTAACTAGATATTTTTCTTTTAATTGC
ATTGCCCCGGAATAAGTAAATTCCATATCCCGTTCGTGCTTAATTTTTGATTCTAAAAATGTAATTTCTT
CTGCTGAATATTTTGACAATAATTCAGGGTCGTATTTACCTGCATTTACACAATAAGAAATATGGTCAAT
AAATGAACGTGGTTCATACTGCCCATAAACATGCTTACGAAGAGCAAACATTAAACAGCGTGCAGCTACA
TATTGATAATCAGGTTCTTCAACCGAAATAGAATTCGCAGCAGCCTTAATGACAATAGTCTGAATATCAT
CAGTTGTCATTCCATCACGGAGATAAGATTTAATATTTTCATATAATTCATAAGGATCTACTGATGTTCC
TTCAGCTGCCCAAGATAAAACTTTAATAATTTTTTGTGGGTCAAAGCTCTGAGAAACACCACTACTTTTG
ATAACATTAATTAATTGCATAAGTCCTCAACTTGAAAATCGTCTTTAAACAATCGGTTAACTATATGAGC
TATTATATCACCATGACACGGCTTTGGTTTACATGTGCATCCTAGCCTCATTCCACGTAAAGGCTCTAAA
TGTGCTTTAGTTATTTCTCCGGATTTAATTCGACGTATAAAATCTTTTTTGAATAATTCAATGGCAGCCT
CCCGGCTGCCAGCATCTTTACCGACGTAATTTCCCCAAAATGTACCGCGGTGAATATTAACATCAAAGTC
GGATTTATATTTATTCACTACCCGGCATAGACGGCCCACGCTGGAATAATTCGTCATATTGTTTTTCCGT
TAAAACAGTAATATCGTAGTAACAGTCAGAAGAAGTTTTAACTGTGGAAATTTTATTATCAAAATACTCA
CGAGTCATTTTATGAGTATAGTATTTTTTACCATAAATGGTAATAGGCTGTTCTGGTCCTGGAACTTCTA
ACTCGCTTGGGTTAGGAAGTGTAAAAAGAACTACACCAGAAGTATCTTTAAATCGTAAAATCATATATCC
TCGCAATAATAAAATTACACCGCCATCTTTCCTTTAATAGGAGGGTGTGATACATAGTTGTTAAGAACGA
AATCTTTAGGCCTAAGTTTAAGAACATATTTTAATTGTTCTTTAGTAGAAAGATATCGGAATTTATAAGG
TAGACCACTTATTACCAGCTCACAAAGCTCTTTAGGTTCACGCCTCAAAATTTCTTTACATTGTTCTACG
TGATTCATATAGATATGAGTATTACCACCAGAAAATATCAAATCCCCTGGAATAAGATTACACATCTTAG
CTACAATATGAACTAACGTAGCATATGACGCAATATTAAACGGTAGCATTATGTTCAGATAAGGTCGTTA
ATCTTACCCCGGAATTATATCCAGCTGCATGTCACCATGCAGAGCAGACTATATCTCCAACTTGTTAAAG
CAAGTTGTCTATCGTTTCGAGTCACTTGACCCTACTCCCCAAAGGGATAGTCGTTAGGCATTTATGTAGA
ACCAATTCCATTTATCAGATTTTACACGATAAGTAACTAATCCAGACGAAATTTTAAAATGTCTAGCTGC
ATCTGCTGCACAATCAAAAATAACCCCATCACATGAAATCTTTTTAATATTACTAGGCTTTTTACCTTTC
ATCTTTTCTGATATTTTAGATTTAGTTATGTCTGAATGCTTATGATTAAAGAATGAATTATTTTCACCTG
AACGATTTCTGCATTTACTACAAGTATAAGCAGAAGTTTGTATGCGAACACCGCACTTACAAAACTTATG
GGTTTCTGGATTCCAACGCCCGTTTTTACTTCCGGGTTTACTGTAAAGAGCTTTCCGACCATCAGGTCCA
AGTTTAAGCATCTTAGCTTTAACAGTTTCAGAACGTTTCTTAATAATTTCTTCTTTTAATGGATGCGTAG
AACATGTATCACCAAACGTTGCATCAGCAATATTGTATCCATTAATTTTAGAATTAAGCTCTTTAATCCA
AAAATTTTCTCGTTCAATAATCAAATCTTTCTCATATGGAATTTCTTCCAAAATAGAACATTCAAACACA
TTACCATGTTTGTTAAAAGACCTCTGAAGTTTTATAGAAGAATGGCATCCTTTTTCTAAATCTTTAAAAT
GCCTCTTCCATCTCTTTTCAAAATCTTTAGCACTTCCTACATATACTTTATTGTTTAAAGTATTTTTAAT
CTGATAAATTCCGCTTTTCATAAATACCTCTTTAAATATAGAAGTATTTATTAAAGGGCAAGTCCTACAA
TTTAGCACGGGATTGTCTACTAGAGAGGTTCCCCGTTTAGATAGATTACAAGTATAAGTCACCTTATACT
CAGGCCTCAATTAACCCAAGAAAACATCTACTGAGCGTTGATACCACTGCAAATCCAAATAGCCATTACG
CACATTAAACTGATAGAACATATGACAAGGCGGTAATGCCATATATTTAAGTTCAGCTGGATTCCATGCA
GAAACAATTTGACGCCTATCATTTGGCAGTTTTTTAATACGATCAATAACTTCTATAATTTGGTCTACAC
CACCAAAATCACGCCACTGTTTTCCATAAATTGGACCAAGTTCACCGCTATGGTATCCTAAATCTTTTGC
TTGATTTTCGTAATTTTCATCCCAGACTGTTTTGCCTTGGATTAACGAATCGTGTTGAATTAATCGTAAA
TCATTGACATTTGTGCTTCCTGATAAAAACCATATTAGCTCAGCAATGCAAGCTTTCCAGGCGAGCTTCT
TAGTTGTTACCGCAGGAAAACCTTTAGTTAAATCCCAGCGTAATTTAGATCCGAACAGAGCAATTGTTCC
TGTGCCTGTACGATCATCGGTTTCATAACCATTTTCAAAAATGTCTTTAATTAAATCTTGGTATTGTTTC
ATTTATATACTGATTCCGTAAGGGTTGTTACTTCATCTATTTTATACCAATGCGTTTCAACCATTTCACG
CTTGCTTATATCATCAAGAAAACTTGCGTCTAATTGAACTGTTGAATTAACACGATGCCTTTTAACGATG
CGAGAAACAACTACTTCATCTGCATAAGGTAATGCAGCATATAACAGAGCAGGCCCGCCAATTACACTTA
CTTTAGAATTCTGATCAAGCATAGTTTCGAATGGTGCATTAGGGCTTGACACTTGAATTTCGCCGCCAGA
AATGTAAGTTATATATTGCTCCCAAGTAATATAGAAATGTGCTAAATCGCCGTCTTTAGTTACAGGATAA
TCACGCGCAAGGTCACACACCACAATATGGCTACGACCAGGAAGTAATGTAGGCAATGACTGGAACGTTT
TAGCACCCATAATCATAATTGTGCCTTCAGTACGAGCTTTAAAATTCTGGAGGTCCTTTTTAACTCGTCC
CCATGGTAAACCATCACCTAAACCGAATGCTAATTCATTAAAGCCGTCGACCGTTTTAGTTGGAGAATAA
CGGAATACCAATTTAATCATTACGTAAATCCTATTTTAATTGAAAACGAATGCTTACTTGGATAATTTCA
ATGACATACATAATATTTTCCTCAAACAGACTTTTTCACAATTTTCCAATCAGCTTTAAACTGCTCGACG
TCAGAATGGTAAATCCAAAATCCTGCGCTTTCTCCGTCTTCATAAAGAGGACATCCATCGCATTCATCTT
CCCATCCCATATCACGTAAAAGATGTTCAGCTTTTTCAACAAGTTCAGAATCTTTACCGATGATATTAAA
ATACCATTTACCTCTAACTTCTGAATCTTTGATGCTCTGGCGTTGTAATCTCATTTTATTCTCCTTAGCA
AGCTTTAATCAAAAGATATAAACAGACCAACATAACTGCTGCCATAATATAAGGTGCGAACATTTTCTTT
TCTCCATTAGTTTTGATAGGGTAATAGTATCACACTACTACCCTGATGTAAACTACTTTTTGAAAGTTTT
TCGCAAAAGTTCAATGATTTCATCTACATTGTTTTCGTCAACAATGCAGTGAATTTTTGTTACGCCAGAA
ACCTTGTCTTTGACTTCATCTTCTTCAGAAGTCGGTTCTTTATATTCGCGGAAACAATAAAACTCTTCTT
CACTAAGTTCAAAATAATCATCACCCATACCATCATCATTATAGATTTCACCATTAGCACAAATGATTTC
GGTTACATAATCAAAGCCATCTAAACTTGATATTGATTTAACTTCAAACCAACCGCCATTTTCTTGAATG
ATGCCGACCATACTAGCATTTGATGAACTAATATCAATGAAAGATTTAATACGATGTGGATTTAACTCGT
ATTTTTTGCCGATTTCCATTTTGATTTTCCTCATTTTAATAGGGGCTTGATAGCCCCTTGATAATTATTG
TTCAATCAGTCCCATGTAAAATTCTGCGTCTTCAGAATCCATGCCATCACAATATTCATTAGCCATAAAG
CGGGTGAGGTCTTCAAGAGGACCTTCAATAACGATTTGAATACTCCAAAACTTAGAATCTTGCACGCTTG
TGATACTAAGTTCAGGATAACGATTACGAATAATCTCTTCAATATATTCAAAATCAACGATGTCAATATC
AACTTTAGCCATATTATTTTCCTCTTTAATTATTAGCAGTATTGCCGATAGTTGTATAGTACCATAAAGC
TTTATGCTTGTAAACCGTTTTGTGAAAAAATTTTTAAAATAAAAAAGGGGACCTCTAGGGTCCCCAATTA
ATTAGTAATATAATCTATTAAAGGTCATTCAAAAGGTCATCCAGGTCCGTGTCATCAGCACTAGATGAAC
TACCAGAGCTTGAGCTCATAAAATCATCTTCAGTTTTTGTATTGAAGTCATCAACATTGAATGCATCCAA
ATCATCAGCCACTTTATCAGCTTTCTTAGCAGCAGTTGCAGCAGCACCGCCCATCACAGCAGTTCCCATA
ACTTGACCGAATTTAGTATTAAGTTCTTCAAACGATTTGAATTTATCTTTAGAAGTCATTTCAGAAAGGT
CAACCATTTGTTCGAACAGTTCTTTCTGGAAAGATTCATCGTCAATGTTTGGAATCGCAGATTGATTCAG
GAATTTAGATTCATCGTAGTTACTAAATCCAGAAACTTGTTTAACTTTCAGTACAAAGTTAGCACCTTCC
CACGGACAAGTTACATCAACTGGAGTTTCACCCATTTCAACATCAACCGCAATCATTGCATTGATTTTAT
CCCAGATTTTCTTACCAAAGCGGTATTTAAATACTTTACCTTCGTTTTCTGGAGCAGCTGGGTCTTTTAC
TACAAGAATGTTAGCCCAGTAAGAAGTTTTACGTTTAACAAGACTGTACTCTTTATTGTCAGTGTTGTAT
AGATCATTTTTACTGATGTATTGACATACTGGGCAAGAATCGTAATCACCATGGGTAGATGAACATGTTT
CAATATACCATTTACCATTTTTCTTGAAACCGTGATTTACAAGAATTGCGAATGGTGCTTGTTCATCATT
TTTAGACGGAAGAAAACGAATTACTGCTTGACCGTTACCCGCATTATCGAGTTTCAGTTTCCACTCGCCT
TTATCTTCAGAAGAAAAACCTTTATTGCCATTCAGTTTAGCCATTTGTGCAGCGAGTTCAGCAGTAGATT
TACGTTTAAACATTTTTATTTCCTTTTTAATTTAATTTAATTAACAGTTGGTGCTATGACACTTTACCTC
ATAGCTGGCATAATTCGCAATACTCTGGGTCTTCGAGAGGTATCCAACCTGAGTTGAAATACTTTACCAT
CGATTTAGCAGTTGTATCAGTTATATTTATATTACCTTTAACTCTTCGCCATCCAGGAGTTTTACCGTAC
AGATTAGAGGATAATAATAACACATAATTCTCGTAAGCAATATGAGATAATTTCCAAGACTCTATATTAG
CTCGTGATGTTTTCCAAGGTCTAAAATCGTCACGGTTCATATAATTAGCCAATCTCATATGCTCTCTAAC
TTCCGGGTCTTTGGCTGGATGAGTTTCACCACTCACACCAAATCCACCACCAGCATATACCAGATTAAAA
TAGTCTGGATTATCTCTGGCATTTACTTCAAGTTGGTATTTTCGTTCTGCTTCAATAACATCCAAGTCAT
CATCAATTTGAATTATTTTAACGCTCGGTTTTTGAAGCATTAGCGCATTCAAAAATCTTTTTTGTTTACA
TGAGCTCCAGTATTCCTTTCCGGAAGAGTCATATATTATTCCGTTCTCAAATGAGCAATTTAATTTACTA
CCGATATAGTAGTATGGCGGAGTCTTATTTTTGACACGGTCTTCAAATGTAAACCAATATACTATATTCA
TATCAATACTTGCAAGATTTCACAGTTTCAATGAAAACATTTTTAGCTTTCTGTGAATCAATATTTAAAA
TTTTTCTATAAGCCTTTAACTTTATAGAATAATTATTCCAGACTAAATTATCAGTCTGTTCATCATGTTT
ATCAATTATATTTAAAAACGAATCAAGCAAGATAAACGTCTCAAACGAAATTATGTTCGATTGCAGAAGT
TTAAAAATATAACTTGATTGAACTTTTGGATTATACTCAAAAATTTCTTTAAAAGCAGAAACTTCAACTT
TTTTACTAAAATAATAAATGTTGCGAATATCTTCTTCAAACTTAAATTTAATTTGCTTTAAGCGTCCGAT
ATATTCACGATAAAACACAAGTGCATCAGCGTCAGAGATGTCACCAATCCAAGCATCTTGGTTAGCAACC
AAATTGCTTATAAAGATTAAAGCAAGTTCCTTTAATTTATATTTTTCTGATAACTTCTGGAAAAAATACT
TATCCCTTCGCTTTTGATAAGCGGCATCAGACACCCGCATGCACCAATTATACTTAATTACATCATACTT
TCCATTCATATGTTGTTTTATCATTAAGTATAATTTATAAACTGATTTACCATCAATGTATCTTTCACCA
CCAGCAGGCATGCGGAGTTTAATCATAGTAGAAAATCTAATGTATTAGTTTTTTCACAACGAACAACAGA
AGGACGTAAAAGATTTTCGTCAATAGCTTCTGACTGAATTTTTTCAATTATACCCGAAGGAATAAATTTA
GCAAATTGAGTTTCAGGAATAGAATTTTCTTCTAAGAATGCTGTTGTAGCTTCAAGATAACTCATTCCAA
ACTCTTCTACCATTTTTTCAATAATAAATCCATTTTCTTGGCGGTCAAGAAGCTTTGCTATTTCATCCTT
TTCTTTCTTAATTGAAAGTTCTTTTTCTGAAAGACCGGTCTCATCGACCGGACGAATATCATTTAGAGAA
AACTGTGTCATAAAGTTCAACTACCTCTTCAGTTTCAGCTTCAAACACATCACGGTTATCTTTATGATAC
AAAGCTAATAGACGATTAAACATCTTACCATCAACGCCAAGTTCATCTTTAGCACGAATTCGAATATCTT
TAATCAGTTCATTATAACCGGAAATTTTCAGTTTATGATCAGATGCTTCTTTAATAAATTTAGCCAAGTC
TTCGCCATGGATAGCTTCATCAAATTCAACCATTTCTTTTTTAGCCATTATTCACCTCAAAATTCATTAA
TGCTATTAGTTAATTTAGAAAGACCCGCTTTTACAAAATATGAATAAATTTTGCCACGCGGTGGTAATTT
ATATGAATTATAGTAATTCACAATGTTTGAAGCAATATTATCAGGAATATAATCAAAATCAATTAGAACT
AAATTTTCTTTATAACGATTATATTCAGATTCAGTGAGAAGCACCTTAGCTTGCTCACGGTCATTAGCAA
TAGCTTCAACGATTGAAGTTTTCATTGAAGGAGTTCGTTCACCTTCAACTCTGGTAAACCAAAAGTCAGA
TCGTACTTTAACTGAAGCAACGTTATCCTTTTTGTCGCCTTTAAGGATTTTAGTCATACAGTCAATTTCA
GCAGAACCGCTTTTAATTTTAACCCATTTCTTATGCATCGGAGACCATTGCTTAACATTTGGATATTTGT
GAAGCTGAGTAAAGTCACCATCTGACGAAATGATTAAAATCTTATGTCCTTCTAAAGAGAACTTTTTAAC
AAGAACAGCAATGTGGTCATCTGCTTCATACTTATCAATATCCATAACAATGTATGGCATATAAGCTTTC
AATTCATCTATAACTTTATGGCTGGATTCAAAATAACCTTCCCAGTCCCAAGTAGATTCTTCTCGTGCTT
TTCCACGGTTTTTCTTATAATAATAAGCGAAATCACGACGCCAATATCCAGATTTCGCGTTATCAATACA
CAGTACAATTTTAGTGTATCCAAGCGTTTTTGCTTTTTTGACATTAAACTTAATTGAGTTCAATATCAAA
TGACGAACCATTGATAAATTAATTTTTTCTTTATCTGGGAAGTTTACCAAAGCAGTTGAAAGCGCAATTT
GACTAAAGTCAATTAAGCAGATTCCTTCTTTGTAATCTTCATCCAACATCATTTCTAAATCCATATGAAC
CTCGTTCAATTAGTGAGATTTCTATTATATACCATCCAAATCTTAAAGTAAACAAGTATAAATACTTATT
ATTGAAAACACAATAGGAGCCCGGGAGAATGGCCGAGATTAAAAGAGAATTCAGAGCAGAAGATGGTCTG
GACGCAGGTGGTGATAAAATAATCAACGTAGCTTTAGCTGATCGTACCGTAGGAACTGACGGTGTTAACG
TTGATTACTTAATTCAAGAAAACACAGTTCAACAGTATGATCCAACTCGTGGATATTTAAAAGATTTTGT
AATCATTTATGATAACCGCTTTTGGGCTGCTATAAATGATATTCCAAAACCAGCAGGAGCTTTTAATAGC
GGACGCTGGAGAGCATTACGTACCGATGCTAACTGGATTACGGTTTCATCTGGTTCATATCAATTAAAAT
CTGGTGAAGCAATTTCGGTTAACACCGCAGCTGGAAATGACATCACGTTTACTTTACCATCTTCTCCAAT
TGATGGTGATACTATCGTTCTCCAAGATATTGGAGGAAAACCTGGAGTTAACCAAGTTTTAATTGTAGCT
CCAGTACAAAGTATTGTAAACTTTAGAGGTGAACAGGTACGTTCAGTACTAATGACTCATCCAAAGTCAC
AGCTAGTTTTAATTTTTAGTAATCGTCTGTGGCAAATGTATGTTGCTGATTATAGTAGAGAAGCTATAGT
TGTAACACCAGCGAATACTTATCAAGCGCAATCCAACGATTTTATCGTACGTAGATTTACTTCTGCTGCA
CCAATTAATGTCAAACTTCCAAGATTTGCTAATCATGGCGATATTATTAATTTCGTCGATTTAGATAAAC
TAAATCCGCTTTATCATACAATTGTTACTACATACGATGAAACGACTTCAGTACAAGAAGTTGGAACTCA
TTCCATTGAAGGCCGTACATCGATTGACGGTTTCTTGATGTTTGATGATAATGAGAAATTATGGAGACTG
TTTGACGGGGATAGTAAAGCGCGTTTACGTATCATAACGACTAATTCAAACATTCGTCCAAATGAAGAAG
TTATGGTATTTGGTGCGAATAACGGAACAACTCAAACAATTGAGCTTAAGCTTCCAACTAATATTTCTGT
TGGTGATACTGTTAAAATTTCCATGAATTACATGAGAAAAGGACAAACAGTTAAAATCAAAGCTGCTGAT
GAAGATAAAATTGCTTCTTCAGTTCAATTGCTGCAATTCCCAAAACGCTCAGAATATCCACCTGAAGCTG
AATGGGTTACAGTTCAAGAATTAGTTTTTAACGATGAAACTAATTATGTTCCAGTTTTGGAGCTTGCTTA
CATAGAAGATTCTGATGGAAAATATTGGGTTGTACAGCAAAACGTTCCAACTGTAGAAAGAGTAGATTCT
TTAAATGATTCTACTAGAGCAAGATTAGGCGTAATTGCTTTAGCTACACAAGCTCAAGCTAATGTCGATT
TAGAAAATTCTCCACAAAAAGAATTAGCAATTACTCCAGAAACGTTAGCTAATCGTACTGCTACAGAAAC
TCGCAGAGGTATTGCAAGAATAGCAACTACTGCTCAAGTGAATCAGAACACCACATTCTCTTTTGCTGAT
GATATTATCATCACTCCTAAAAAGCTGAATGAAAGAACTGCTACAGAAACTCGTAGAGGTGTCGCAGAAA
TTGCTACGCAGCAAGAAACTAATGCAGGAACCGATGATACTACAATCATCACTCCTAAAAAGCTTCAAGC
TCGTCAAGGTTCTGAATCATTATCTGGTATTGTAACCTTTGTATCTACTGCAGGTGCTACTCCAGCTTCT
AGCCGTGAATTAAATGGTACGAATGTTTATAATAAAAACACTGATAATTTAGTTGTTTCACCTAAAGCTT
TGGATCAGTATAAAGCTACTCCAACACAGCAAGGTGCAGTAATTTTAGCAGTTGAAAGTGAAGTAATTGC
TGGACAAAGTCAGCAAGGATGGGCAAATGCTGTTGTAACGCCAGAAACGTTACATAAAAAGACATCAACT
GATGGAAGAATTGGTTTAATTGAAATTGCTACGCAAAGTGAAGTTAATACAGGAACTGATTATACTCGTG
CAGTCACTCCTAAAACTTTAAATGACCGTAGAGCAACTGAAAGTTTAAGTGGTATAGCTGAAATTGCTAC
ACAAGTTGAATTCGACGCAGGCGTCGACGATACTCGTATCTCTACACCATTAAAAATTAAAACCAGATTT
AATAGTACTGATCGTACTTCTGTTGTTGCTCTATCTGGATTAGTTGAATCAGGAACTCTCTGGGACCATT
ATACACTTAATATTCTTGAAGCAAATGAGACACAACGTGGTACACTTCGTGTAGCTACGCAGGTCGAAGC
TGCTGCGGGAACATTAGATAATGTTTTAATAACTCCTAAAAAGCTTTTAGGTACTAAATCTACTGAAGCG
CAAGAGGGTGTTATTAAAGTTGCAACTCAGTCTGAAACTGTGACTGGAACGTCAGCAAATACTGCTGTAT
CTCCAAAAAATTTAAAATGGATTGCGCAGAGTGAACCTACTTGGGCAGCTACTACTGCAATAAGAGGTTT
TGTTAAAACTTCATCTGGTTCAATTACATTCGTTGGTAATGATACAGTCGGTTCTACCCAAGATTTAGAA
CTGTATGAGAAAAATAGCTATGCGGTATCACCATATGAATTAAACCGTGTATTAGCAAATTATTTGCCAC
TAAAAGCAAAAGCTGCTGATACAAATTTATTGGATGGTCTAGATTCATCTCAGTTCATTCGTAGGGATAT
TGCACAGACGGTTAATGGTTCACTAACCTTAACCCAACAAACGAATCTGAGTGCCCCTCTTGTATCATCT
AGTACTGGTGAATTTGGTGGTTCATTGGCCGCTAATAGAACATTTACCATCCGTAATACAGGAGCCCCGA
CTAGTATCGTTTTCGAAAAAGGTCCTGCATCCGGGGCAAATCCTGCACAGTCAATGAGTATTCGTGTATG
GGGTAACCAATTTGGCGGCGGTAGTGATACGACCCGTTCGACAGTGTTTGAAGTTGGCGATGACACATCT
CATCACTTTTATTCTCAACGTAATAAAGACGGTAATATAGCGTTTAACATTAATGGTACTGTAATGCCAA
TAAACATTAATGCTTCCGGTTTGATGAATGTGAATGGCACTGCAACATTCGGTCGTTCAGTTACAGCCAA
TGGTGAATTCATCAGCAAGTCTGCAAATGCTTTTAGAGCAATAAACGGTGATTACGGATTCTTTATTCGT
AATGATGCCTCTAATACCTATTTTTTGCTCACTGCAGCCGGTGATCAGACTGGTGGTTTTAATGGATTAC
GCCCATTATTAATTAATAATCAATCCGGTCAGATTACAATTGGTGAAGGCTTAATCATTGCCAAAGGTGT
TACTATAAATTCAGGCGGTTTAACTGTTAACTCGAGAATTCGTTCTCAGGGTACTAAAACATCTGATTTA
TATACCCGTGCGCCAACATCTGATACTGTAGGATTCTGGTCAATCGATATTAATGATTCAGCCACTTATA
ACCAGTTCCCGGGTTATTTTAAAATGGTTGAAAAAACTAATGAAGTGACTGGGCTTCCATACTTAGAACG
TGGCGAAGAAGTTAAATCTCCTGGTACACTGACTCAGTTTGGTAACACACTTGATTCGCTTTACCAAGAT
TGGATTACTTATCCAACGACGCCAGAAGCGCGTACCACTCGCTGGACACGTACATGGCAGAAAACCAAAA
ACTCTTGGTCAAGTTTTGTTCAGGTATTTGACGGAGGTAACCCTCCTCAACCATCTGATATCGGTGCTTT
ACCATCTGATAATGCTACAATGGGGAATCTTACTATTCGTGATTTCTTGCGAATTGGTAATGTTCGCATT
GTTCCTGACCCAGTGAATAAAACGGTTAAATTTGAATGGGTTGAATAAGAGGTATTATGGAAAAATTTAT
GGCCGAGTTTGGACAAGGATATGTCCAAACGCCATTTTTATCGGAAAGTAATTCAGTAAGATATAAAATA
AGTATAGCGGGTTCTTGCCCGCTTTCTACAGCAGGACCATCATATGTTAAATTTCAGGATAATCCTGTAG
GAAGTCAAACATTTAGCGCAGGCCTCCATTTAAGAGTTTTTGACCCTTCCACCGGAGCATTAGTTGATAG
TAAGTCATATGCCTTTTCGACTTCAAATGATACTACATCAGCTGCTTTTGTTAGTTTCATGAATTCTTTG
ACGAATAATCGAATTGTTGCTATATTAACTAGTGGAAAGGTTAATTTTCCTCCTGAAGTAGTATCTTGGT
TAAGAACCGCCGGAACGTCTGCCTTTCCATCTGATTCTATATTGTCAAGATTTGACGTATCATATGCTGC
TTTTTATACTTCTTCTAAAAGAGCTATCGCATTAGAGCATGTTAAACTGAGTAATAGAAAAAGCACAGAT
GATTATCAAACTATTTTAGATGTTGTATTTGACAGTTTAGAAGATGTAGGGGCTACCGGGTTTCCAAGAG
GAACGTATGAAAGTGTTGAGCAATTCATGTCGGCAGTTGGTGGAACTAATGACGAAATTGCGAGATTGCC
AACTTCAGCTGCTATAAGTAAATTATCTGATTATAATTTAATTCCTGGAGATGTTCTTTATCTTAAAGCT
CAGTTATATGCTGATGCTGATTTACTTGCTCTTGGAACTACAAATATATCTATCCGTTTTTATAATGCAT
CTAACGGATATATTTCTTCAACACAAGCTGAATTTACTGGGCAAGCTGGGTCATGGGAATTAAAGGAAGA
TTATGTAGTTGTTCCAGAAAACGCAGTAGGATTTACGATATACGCACAGAGAACTGCACAAGCTGGCCAA
GGTGGCATGAGAAATTTAAGCTTTTCTGAAGTATCAAGAAATGGCGGCATTTCGAAACCTGCTGAATTTG
GCGTCAATGGTATTCGTGTTAATTATATCTGCGAATCCGCTTCACCCCCGGATATAATGGTACTTCCTAC
GCAAGCATCGTCTAAAACTGGTAAAGTGTTTGGGCAAGAATTTAGAGAAGTTTAAATTGAGGGACCCTTC
GGGTTCCCTTTTTCTTTATAAATACTATTCAAATAAAGGGGCATACAATGGCTGATTTAAAAGTAGGTTC
AACAACTGGAGGCTCTGTCATTTGGCATCAAGGAAATTTTCCATTGAATCCAGCCGGTGACGATGTACTC
TATAAATCATTTAAAATATATTCAGAATATAACAAACCACAAGCTGCTGATAACGATTTCGTTTCTAAAG
CTAATGGTGGTACTTATGCATCAAAGGTAACATTTAACGCTGGCATTCAAGTCCCATATGCTCCAAACAT
CATGAGCCCATGCGGGATTTATGGGGGTAACGGTGATGGTGCTACTTTTGATAAAGCAAATATCGATATT
GTTTCATGGTATGGCGTAGGATTTAAATCGTCATTTGGTTCAACAGGCCGAACTGTTGTAATTAATACAC
GCAATGGTGATATTAACACAAAAGGTGTTGTGTCGGCAGCTGGTCAAGTAAGAAGTGGTGCGGCTGCTCC
TATAGCAGCGAATGACCTTACTAGAAAGGACTATGTTGATGGAGCAATAAATACTGTTACTGCAAATGCA
AACTCTAGGGTGCTACGGTCTGGTGACACCATGACAGGTAATTTAACAGCGCCAAACTTTTTCTCGCAGA
ATCCTGCATCTCAACCCTCACACGTTCCACGATTTGACCAAATCGTAATTAAGGATTCTGTTCAAGATTT
CGGCTATTATTAAGAGGACTTATGGCTACTTTAAAACAAATACAATTTAAAAGAAGCAAAATCGCAGGAA
CACGTCCTGCTGCTTCAGTATTAGCCGAAGGTGAATTGGCTATAAACTTAAAAGATAGAACAATTTTTAC
TAAAGATGATTCAGGAAATATCATCGATCTAGGTTTTGCTAAAGGGGGGCAAGTTGATGGCAACGTTACT
ATTAACGGACTTTTGAGATTAAATGGCGATTATGTACAAACAGGTGGAATGACTGTAAACGGACCCATTG
GTTCTACTGATGGCGTCACTGGAAAAATTTTCAGATCTACACAGGGTTCATTTTATGCAAGAGCAACAAA
CGATACTTCAAATGCCCATTTATGGTTTGAAAATGCCGATGGCACTGAACGTGGCGTTATATATGCTCGC
CCTCAAACTACAACTGACGGTGAAATACGCCTTAGGGTTAGACAAGGAACAGGAAGCACTGCCAACAGTG
AATTCTATTTCCGCTCTATAAATGGAGGCGAATTTCAGGCTAACCGTATTTTAGCATCAGATTCGTTAGT
AACAAAACGCATTGCGGTTGATACCGTTATTCATGATGCCAAAGCATTTGGACAATATGATTCTCACTCT
TTGGTTAATTATGTTTATCCTGGAACCGGTGAAACAAATGGTGTAAACTATCTTCGTAAAGTTCGCGCTA
AGTCCGGTGGTACAATTTATCATGAAATTGTTACTGCACAAACAGGCCTGGCTGATGAAGTTTCTTGGTG
GTCTGGTGATACACCAGTATTTAAACTATACGGTATTCGTGACGATGGCAGAATGATTATCCGTAATAGC
CTTGCATTAGGTACATTCACTACAAATTTCCCGTCTAGTGATTATGGCAACGTCGGTGTAATGGGCGATA
AGTATCTTGTTCTCGGCGACACTGTAACTGGCTTGTCATACAAAAAAACTGGTGTATTTGATCTAGTTGG
CGGTGGATATTCTGTTGCTTCTATTACTCCTGACAGTTTCCGTAGTACTCGTAAAGGTATATTTGGTCGT
TCTGAGGACCAAGGCGCAACTTGGATAATGCCTGGTACAAATGCTGCTCTCTTGTCTGTTCAAACACAAG
CTGATAATAACAATGCTGGAGACGGACAAACCCATATCGGGTACAATGCTGGCGGTAAAATGAACCACTA
TTTCCGTGGTACAGGTCAGATGAATATCAATACCCAACAAGGTATGGAAATTAACCCGGGTATTTTGAAA
TTGGTAACTGGCTCTAATAATGTACAATTTTACGCTGACGGAACTATTTCTTCCATTCAACCTATTAAAT
TAGATAACGAGATATTTTTAACTAAATCTAATAATACTGCGGGTCTTAAATTTGGAGCTCCTAGCCAAGT
TGATGGCACAAGGACTATCCAATGGAACGGTGGTACTCGCGAAGGACAGAATAAAAACTATGTGATTATT
AAAGCATGGGGTAACTCATTTAATGCCACTGGTGATAGATCTCGCGAAACGGTTTTCCAAGTATCAGATA
GTCAAGGATATTATTTTTATGCTCATCGTAAAGCTCCAACCGGCGACGAAACTATTGGACGTATTGAAGC
TCAATTTGCTGGGGATGTTTATGCTAAAGGTATTATTGCCAACGGAAATTTTAGAGTTGTTGGGTCAAGC
GCTTTAGCCGGCAATGTTACTATGTCTAACGGTTTGTTTGTCCAAGGTGGTTCTTCTATTACTGGACAAG
TTAAAATTGGCGGAACAGCAAACGCACTGAGAATTTGGAACGCTGAATATGGTGCTATTTTCCGTCGTTC
GGAAAGTAACTTTTATATTATTCCAACCAATCAAAATGAAGGAGAAAGTGGAGACATTCACAGCTCTTTG
AGACCTGTGAGAATAGGATTAAACGATGGCATGGTTGGGTTAGGAAGAGATTCTTTTATAGTAGATCAAA
ATAATGCTTTAACTACGATAAACAGTAACTCTCGCATTAATGCCAACTTTAGAATGCAATTGGGGCAGTC
GGCATACATTGATGCAGAATGTACTGATGCTGTTCGCCCGGCGGGTGCAGGTTCATTTGCTTCCCAGAAT
AATGAAGACGTCCGTGCGCCGTTCTATATGAATATTGATAGAACTGATGCTAGTGCATATGTTCCTATTT
TGAAACAACGTTATGTTCAAGGCAATGGCTGCTATTCATTAGGGACTTTAATTAATAATGGTAATTTCCG
AGTTCATTACCATGGCGGCGGAGATAACGGTTCTACAGGTCCACAGACTGCTGATTTTGGATGGGAATTT
ATTAAAAACGGTGATTTTATTTCACCTCGCGATTTAATAGCAGGCAAAGTCAGATTTGATAGAACTGGTA
ATATCACTGGTGGTTCTGGTAATTTTGCTAACTTAAACAGTACAATTGAATCACTTAAAACTGATATCAT
GTCGAGTTACCCAATTGGTGCTCCGATTCCTTGGCCGAGTGATTCAGTTCCTGCTGGATTTGCTTTGATG
GAAGGTCAGACCTTTGATAAGTCCGCATATCCAAAGTTAGCTGTTGCATATCCTAGCGGTGTTATTCCAG
ATATGCGCGGGCAAACTATCAAGGGTAAACCAAGTGGTCGTGCTGTTTTGAGCGCTGAGGCAGATGGTGT
TAAGGCTCATAGCCATAGTGCATCGGCTTCAAGTACTGACTTAGGTACTAAAACCACATCAAGCTTTGAC
TATGGTACGAAGGGAACTAACAGTACGGGTGGACACACTCACTCTGGTAGTGGTTCTACTAGCACAAATG
GTGAGCACAGCCACTACATCGAGGCATGGAATGGTACTGGTGTAGGTGGTAATAAGATGTCATCATATGC
CATATCATACAGGGCGGGTGGGAGTAACACTAATGCAGCAGGGAACCACAGTCACACTTTCTCTTTTGGG
ACTAGCAGTGCTGGCGACCATTCCCACTCTGTAGGTATTGGTGCTCATACCCACACGGTAGCAATTGGAT
CACATGGTCATACTATCACTGTAAATAGTACAGGTAATACAGAAAACACGGTTAAAAACATTGCTTTTAA
CTATATCGTTCGTTTAGCATAAGGAGAGGGGCTTCGGCCCTTCTAAATATGAAAATATATCATTATTATT
TTGACACTAAAGAATTTTACAAAGAAGAAAATTACAAACCGGTTAAAGGCCTCGGTCTTCCTGCTCATTC
AACAATTAAAAAACCTTTAGAACCTAAAGAAGGATACGCGGTTGTATTTGATGAACGTACTCAGGATTGG
ATTTATGAAGAAGACCATCGCGGAAAACGCGCATGGACTTTTAATAAAGAAGAAATTTTTATAAGTGACA
TTGGAAGCCCGGTTGGTATAACTTTCGATGAGCCCGGCGAATTTGATATATGGACTGATGACGGTTGGAA
AGAAGACGAAACATATAAGCGAGTTTTAATTCGTAATAGAAAAATTGAAGAATTATATAAAGAGTTCCAA
GTTTTAAATAATATGATTGAAGCTTCTGTCGCCAATAAAAAGGAAAAATTCTATTATAAAAACCTTAAGC
GGTTCTTTGCTCTTTTAGAAAAGCATGAGCATTTAGGTGGTGAATTCCCTTCATGGCCTGAAAAAGAACA
GAAGCCTTGGTATAAGCGTTTATTCAAGCATTACGTATAAATATCTTAAAAGGAGGGTCTATGGCAGCAC
CTAGAATATCATTTTCGCCCTCTGATATTCTATTTGGTGTTCTAGATCGCTTGTTCAAAGATAACGCTAC
CGGGAAGGTTCTTGCTTCCCGGGTAGCTGTCGTAATTCTTTTGTTTATAATGGCGATTGTTTGGTATAGG
GGAGATAGTTTCTTTGAGTACTATAAGCAATCAAAGTATGAAACATACAGTGAAATTATTGAAAAGGAAA
GAACTGCACGCTTTGAATCTGTCGCCCTGGAACAACTCCAGATAGTTCATATATCATCTGAGGCAGACTT
TAGTGCGGTGTATTCTTTCCGCCCTAAAAACTTAAACTATTTTGTTGATATTATAGCATACGAAGGAAAA
TTACCTTCAACAATAAGTGAAAAATCACTTGGAGGATATCCTGTTGATAAAACTATGGATGAATATACAG
TTCATTTAAATGGACGTCATTATTATTCCAACTCAAAATTTGCTTTTTTACCAACTAAAAAGCCTACTCC
CGAAATAAACTACATGTACAGTTGTCCATATTTTAATTTGGATAATATCTATGCTGGAACGATAACCATG
TACTGGTATAGAAATGATCATATAAGTAATGACCGCCTTGAATCAATATGTGCTCAGGCGGCCAGAATAT
TAGGAAGGGCTAAATAATTATTTGTTCGTATACATCTCTAGATATCGATATACACCCTCAAAACCCTCGT
TGAATTCGTCGATGAGGGTTTTCTTATCTTCTTGAGTTAATTCAGAAACAATTTTACGGAATGAATTTTG
ATTTAACTTTCTACCTTCATGCGTTACTCCAATCTCATTTAGAAATGCAATAAAATTAGCACGATTCTCA
ACAATATCTTCTCTGGAAAATTTAATCAAAATAGATGCAACAGTAATAATTTCACGAACTGTATCAATGT
TTTTATTCATTAACTATACCACTCAATTAGTTGACTTTGTTATAATATCATCAGACGCTTGATTTGTAAA
CTGGTCTGTGTAATTTTCTTCAAAAATTTTTTCTACGAATTCCTTGAACGATTCACGTTCCTGAGCTACA
TTATGCTCGATTACCTTTTCAAGATTATGACTCATTCGAAATAATCTTCAATTTCATAATCATGGACATA
AATCATTATAGTTTTTAATACATCATCAATATTTTTTCCTGGAGCTGGAATTACGTAAAAATACCCTGCT
TTTGAGAGGTCTTTATAAGTTCCAATCAAGAAATCATTATTCTCAAGATGTAACTCTTCAACTAATTCAT
TGACAATTGAATGGTATAGGTTTGGCAGAAACTTATATAGCTTTTCTAGAATATCAATTTTGAATGTATA
TTGAACCACGGACTGAGAATCAATAATCATAGACCTTCCCCTTATGTTTCTGTTTGCGATTAGATTCTTT
AAACGCTTTCTTCTTATCCTTATGAACAGAAGCTTTATTAAAATTATGCTTTGCGACTAAATTGTTCATA
GTGCTGAATTACCTCTCTTAAACATTTGCATGTGAATGAAAACTTTTTAGCTACACCACATTCAAATATA
TGTTCTCTTAAATCGCGTGTATCGGTATATCCCATCTCAACAATAAAATGCCGTATTAGATTTTTATCTT
TATCGTTTAGAGAATTAAAATAATCAGATTTTGAATTAATTTCCCTGGCCAAATTGAATCACCTTCAGTT
GACGTTTTAACTCTTTTATCATCTCTTCGTTCATCGCAATATAAAGATCGCGTAGAGCAGGTTTTAGCAT
TCCATTTACTGGAGAACTAAATGGACATACATAATCTTTTCCTACGAGCTTTTTAGTGAATTCCATATCA
CAGAACTGAAATCCCGGCTCATTGGTATAAATTCCCCAATTAGTTGACATCATTTTATTGGCATATTCCA
GTGCCTGGATTTGATTCATAATTCCATCAATTTGAAACTTTTTAATATTCATTAGTAAAGGTCCTCAGAG
TAAAGTTCTTTTTCACTACCACGTTCAATACTTACTTGTCCAGCGTAAGTTGCAATAATCATTGCTTCTT
CACGTGTCCAATAATTACTATATTGGTCAATAAACCCTTGGTCATCATCACAAACTTGCTGAGTAACTAA
TTGAGGTTTAACTACATCTAAAACTTCTGCCATATCTTTAGAATAATGACGAGCACCTGGAATAATAAGA
GTTCGTCCATCTTTTAATTTAAAACGGTTGGCTGCGCAAACAATTCGTCGTTGATACTTTTGGTTTTCAT
CCCAGTACGCAGTCTGCCAACAGATTTCAGGAACTTCTTTCAGAATATCTTCTTCTGTGCATTTATAACC
ATGCGCTTTAAATTTTTCAACAAGACTTTCTGGAGTTTCACGAGATAAAGGAACATTCAGCATTTTTAAA
CGATTGATAAATGGGTTCATTTAAACCATCCTTTAATACGCTGCCACAAAGTTTTCTGTTGAGCTTTGTT
GACGCCAATTGAGCGAATAACCGGTTGAGATTCCTGGAATTCTTTATAATCAGCAAGGTAAATTTCGTAA
GCTGCATCCGTAAATGAACTTATCGCTGCCATAAAATTATTGCGAATACCTACTGGAGCATCTTTACTTT
CACGAATGATCATGTATTTACCAGTCTTAATCTTTACGATAGTTCCAAGATAAGCTCCATGGTACCAAAT
ATCCCAACCCTCTTGAGTAGGTTCTGCGCAACGACGAAGTTCATTGACAATTTCTAACTTGTTTATTATT
TATTCCTCACAGTTCAGATGCTACAGTGATTACAGCTTCAATGTTTTCTGCCGAGCGTTTAATGTCAAGA
TACACATTACCGTTTTTAGCGATTTTACATGACATTCCGATGTCAGTAAATTTCTGAATATGATGTTCCA
TCATTTTGTATCCAAAAATTCGCATATTTCCATTGTTATTAATTTCAAAATTACGAATTCCGTTAGTGCG
TTTTTCTAAAATAGCAAGATAATTACTACGATAAATTTCAACCTTTTTAAGAACAAATCCATTTTTATCT
AAAAGTTTTAACATGAGGTCTTTATCTTCTTCCATATCGGAAGTAATCTCGCGAGCTTTACGAGTTGCTC
GTTTTTTCAGCAGTTCCGGAGCATTTTCCTGTGCATATAAAGTTGCTGCATTTGAAATAATATCCTGAGC
TTCACCAGTAATGATTAATCCATCACCAGATTTCTCCACCAGGCCTTTTTTAATCAATACCCCAATATTA
CTATTAACTACTGCGTTACCTAAATCTGGATGCACCTCACGAACTTCTGCAGCTGTAATGAAATCTTTCT
TAGCAATGGTAATTAAAATCGTAGCAGTTTTTTCATTCAGAACATCGTTAGAAGCTTTGATGATGTAAGT
TACTTTAGACATTTTCTAATCTCCGTAATTCTGTATCAGTAGTTGATAGTTGTATAGTACCACAGTATGC
TTTGGTTGTAAACCGTTTTGTGAAAAAATTTTTAAAATAAAAAAGGGAGAGCCTCGGCTCTCCCTAAAAT
TACTGCATGACTGTGATAACTGTCATGATAACACGTTGAATTCCGAACGCAAGAAGACCTCCTGCTACGG
CTGGAACAACGCCTAAACCCGCCAGTAAAATGCTACCAGATACTAATGCAGCGCTTGTAATACCAATGAA
TGGACTCATTTGATTTCCTCTAAATCTTTGGTGTATTCAGTAACTACATCAGTAGTTTTCCAATATTCGT
TTTCTTCTTTTTTAGCTTTAGCTTCTTCAGCAAGTTTCTTTGCTTCATCGGAAGTCATATGAAAAATATT
CATTCCAACTAGTTTATCAACATAAGAAGAATACATATCGATTTTCGAAAGTTCTTCGGTCAGTTCTTTA
CGAGTTTTACCCTGTACAACAATTTCACCTGAAATTACTTTCTTAATGAAATGTGCTTTGGCAAAGGCTA
AACGAAAAGCTGACTCAGTTTCTTTAATTTTGTTATCAATTCGTTTTTGGACATAAGTTTTACGAACTTC
AACAAAGTCTTTAATTAAATCAACTACGTTATCGTAAACTTGCAGCTTTCCTTTCTCATTAATAACCGTA
ATATTCTGGGAACGACGCTCAATCAGTCCGAAGTCTTTCATAATTTTTGCATGGCGTTCTTCTTCGTTAT
CGCTCAAAGAATATTCTTTGCGGAATTTAACTTTGAAGCCAAAACCATGCTCACCACAAGCATCATCCCA
TGTAATGAAGCCTTTATTTTCAAGTGGGTCTAAGATTTTACTCACATAAGTTTCACGATCATACTTATAC
GGAATCTCAGTGATATGCATTTGAGTTCGTGAAGTAAACTTATATGTTCCACGAATTTCATATTGCCCAT
CAATTTCAACGACTTCACCACGAAATTCTGGGAATTCTACTTTCGGTTTAGTTACTTTCTTTCCTTGAAG
AGCTTGCAGTACAGCTTTCTTGACAGAAGAAACACTATGAGGAAGAATGTAAGTTGCATAACCAGTTGCA
ATACCGGAAACGCCATTAAGAAGAACAGTAGGAATAATAGGCAAATAGAAAGCAGGCGGAATGTGTTCTT
TATCTTGATGTACCGGAGCATATTCAGTATCTTTATATACGTTATAGAAATTTTTACTTACACGAGCAAA
AATATAACGACTTGCCGCTGCCTTTTGGACAGTACGAGAACCAAAGTTTCCTTGACCATCTAACAGAGGA
AAGTTATTATTCCAAGTATTAGCCATCAAAGCACCTGCGTCTTGTGCAGAGTTTTCACCATGATGATATC
CAAGGTCCGCTACACCACCTGCAATAGAAGCGAGTTTGTGAAACTTATCTTTATTTCCTCGTGCCAAATC
AAGAGCTCGAGCAATAACAAATCGTTGAACTGGCTTAAATCCATCAATCATATTTGGGATAGCACGATTT
TCAACCGTGTACATAGCATAAGCCAATGCTTCATTATCAATGATACTTTTTAAATCGCGATTATTCAGTT
GCATAAATTTACCATACTAGTGAATGTAGTGCCATAATAACATCAGAAATGAAAAGCACGACTTGAATTA
ATCCGAACATTACTCCGTAATATAGTGCTACCAATAAAGCAGCAAGGGCTAATGAATAGCCCAAGATTTT
CTTAATCATTAGATAACAACACAAATGTTAAATATGCACACATACCCTGGGCTAAAGCTTGTGAAAACAC
ACTGCTAGCATCGATACAGATAGTTAAAACACATGCTACTATCCAACAAATAAATGAAATAACTCCTAAT
AATTTTGCAATATTCATATTTTCCTCACTGGCGTCCGAAGACGCCTTTAGTTTTAAGATTGTTACGATAG
AACTGCATCACGTGTTCGTTATGGAAATTACTCATTAATATGCCTGTAAAACAAATTTAAAGTTATCAGC
CAACATACGGTTCATTTCTTCGAGTGTTTGATACTCAGAATGATGATTACGAGTAAACGCCAAAGCTAGC
TGACCTTTTCCAAATCCCGTCGTTAGAGGTTTCATCTTAGAAGCAGGCAGATAAAACACTGTGTATGGAA
CATTGTTATTTGCAATAGTACGCGCAAGTTGAGACCGGCGTTGACGAATATGACTTAAAACCGCACTGAA
TCCTTGCTTAGAACGCTGATTACCTACATAAAATCGTGCAGATACGCATGGATTACTAAATGGACCACCG
AGTTTACTCACTAAAAAGTAAAATCCAGGTTTAGATAAAATATCTTTATGCGGAGTTCCTAAAAACCATT
CACCACCCTTGATTGTACCAATAACAGTAGCGCCTGCATCATTCAGATCAGTAACAGTCATATATTTCAT
ATTAATTTCCTCTAAATTATTTTCTACTCCAAGGCCGCATGAATACACACGGCCATTAAATTACTCGTCG
CAGTCGACGCTCAATTCCCAAAACTCTTCTACAGTATAAGTTTCAGTATCATTTTCAATACAGAAACGTT
CATTACTATTATTTGCTAAAGTAGCATTAACTGTCATTTTTTCGCTAGTGCTCTTAAGAGGTGAAATACG
AATTAACTGATCACCGTTATCTAAACAAAAAATTTCACCAACTTTTACATCTTTAAAACTTTTCATAATT
CACCTCAAGGAGTATAAAATCCAAATGCAGTTGTTGACCATCCCATCCAATATGGAAAATTTACACCAAT
GTAAAACATAAGAATATAAAACCAACCGCTCAGCAAATTCATCATTTTACACCATTCCAAATTGTTTCAA
CCACGGATTTTAAACCATTTTGATGAATATCCATTCCTACTACCGCCATCAAATAAATTCCAACTACAAC
TGAACCTAAGGCAAAAATCAGCATGAAAATGAATAAAGCCGGAAAAATATTATCGAAAAACCATTCAATA
AATGTAAAAGCACTGCGTTTACGTTCATATTTTCCTCACATAAATCCAAAGTAAACGTTTAATACATCAA
TCATTAAAACGATTGGGAATATACTCAAAACTATTAGTATTATAACTACATTCCATATAGCTTTAATAAT
CTTTTTCATTTTCTGTTCCTCCATAGTTGATAGGGTAATAGTACCACGGAAGAACAGTCTTGTAAACAAC
TTTTTTAAAAATATTCGTAATAAATGTGAATACCAACTACTACCGCTGAAACCTGTGCAACCCACCACGC
ACAAGCAATAAGTACAGAATTCAAAATTTTCATAATAACCTCATTACAAAAGTAAATGTTAAACAAATTA
CTGGAATACTAATTAACCAAACAAAACACCACCATAATGAACTCATAGTTCAATCTCAGCGATTTTCATT
TTATTCTCCAAATCCGTATCAGTAGTTGATAGTTGTATAGTACCACGGTCCTTGTGGTATGTAAACTGTT
TTGTGAAATTTTTTAAATGGAAAGATACCATCCGTTGTAGTTGCTTTTTCTTACAACTTTACGAAGGTCT
TCTCTGTCACCGATGAACTTCGGAGTGTACTGGATGACACCTGGATGAATTTCTTTAGTGTTGAATATAA
TTATACAGTCAGCGACTTGATGATTTAGAATGGGCCCTAGATTTATTCCAGAACCATATGGATACTCTCC
GCTGCATCCCGTTGTTACCGAAATCCAACGTGAGTCAGTTTGATGTGTCTTAACTTCTACACGAAGCCCG
CAGTATTTTGGATGAGCCAATACATCCCATGCATATGTGTACGGATCATCGACATCCTCTTGGCCTTTAT
TGACATATCCACTCAACCAATCTGCCACAAAAAACTCTGCGTACACAGCGATACGGCATCTTTCGATAAC
TTCTGCCTTATCTTGATTTGGGTTTTGTTTTAAAGAGTATCTTGCAGTATCAGCAATTTTGACCTTCATT
TCACAGGTCAAGTCACTGTTCGATAGGGTAAATGTCGGAATCTGAAATAGTCTCTGTAACCCAGGATTCG
TTTTCTGCATTTAAACTTTCCTTTATGTCGGAATCACCGATATTCATATAAATCATAATTTCTCTTAAAA
CAAAAGGCCGAAGCCCTTTATTTTACTTGAATTGTGCAATTCTTTTCTCTAGACATTCAGCATAAGATTT
CATTGAGATGAACTGCGAAAGTAGCAGTTCTTGCTCAACTGCGCTAACTGTTAGAAACTTTGCGCTTTCT
AAAAATTTGCTCAGTGCATTAATTTTGAGCATTAATTGATCGTATTCTTCTTTTACTCGTGCTTGATAAG
CTAACATAATTTTCCTTAGTTAAGGGCCGAAGCCTTATTTAAATTGTTCAGTAACGTCTTCAACTACTTC
ATATTGGCAGGTACGCATTTTAGCATCGTTGTAATCAATCGGAATTGATACTACATCGCGAGGATGAACT
TTAACTTTTACAACTCGGCTGGTTGAACTACCAAAGTGACGAATATAAGATTTAGAACACACATGCAAAC
CACGAGAACAAGTTTGTGTATCATCGTCATTCACACGAGTACGTGGCATTTTAACTACTTTACCCGGACT
GTTATCAAAGGTGTTTGAGTGACAGTCAAAGTAATTGCTGCGAACTACTTTCCAAGCATAGAAGTAACCA
TCTTCTGTAATTTCAATATCGTTTGCTACCAAGAAATCAAAGAGTCGAGATACCGCTTTTTGGCTTGGGT
TTTCCAACAGATTTTCCAAGAACGGAAAATAAAATTCAAAGTTTTCGCCTTTTTCCATCGAGTCAAGAAT
ACGATCAACCAAACCAGACCGCAATTCAATATTTTGATAGAACAAGCTTCCACCTTCAATTCGAACATCG
CCGGAAATATATTTTTCAACAGCACGACGAACATTAATTTTTTGTGCCGCTTCTTCCAACTTATCCGCTA
CAAGCAGATTAAGAATTTCCTGGAAGTTTGAATGAGTATTAGGAGTTGCGTTATAAGTTACGCCATCAAC
AGTAATTGAAATGAATTTTTTAGATGCATTCCAAATAATGTCAGATTTAGCAACTGGAGCAATAACTGCA
TCGCTATTAACTTTAACTGTAATATCACCGCTAATAGTAACTTTAGGGCGTTTAGCTTCTTCAGCATTTT
TCAAAACACGACGGATTGTGTCAACCGATACACCTTGCCAATCAGCCAATTCCTGTTGGGTGTAATTACC
ACTTGAATACAGTTTAACAATTTCAGCTTGTTCGTTTTTGGTCAGGCATTTAATATTGTACAT

1: A synthetic phage that is capable of replication in a host cell, wherein the phage is
(a) (i) a synthetic T-even phage comprising (a) a deletion of DNA from, and/or (b) an insertion into, a Deletion Permissive Region (DPR) of the genome of the phage, wherein the region is between the pin (protease inhibitor) gene and the iPII (internal protein) gene; or (ii) a synthetic version of a phage that is not a T-even phage, wherein the synthetic phage comprises a deletion of DNA from a region of its genome that is homologous or orthologous to said DPR of (i); or
(b) (iii) a synthetic rV5 or rV5-like phage comprising (a) a deletion of DNA from, and/or (b) an insertion into, a Deletion Permissive Region (DPR) of the genome of the phage, wherein the region is between gene 39 and gene 46 or between gene 230 and gene 240; or (iv) a synthetic version of a phage that is not a rV5 or rV5-like phage, wherein the synthetic version of the phage comprises a deletion of DNA from a region of its genome that is homologous or orthologous to said DPR of (iii).

2-12. (canceled)

13: A DNA comprising the genome of the synthetic phage of claim 1 optionally wherein the DNA is a chromosome of a bacterial cell or a plasmid comprised by a bacterial cell, such as a host cell of said synthetic phage.

14: The DNA of claim 13, wherein the heterologous DNA comprises or encodes
A. one or more components of a CRISPR/Cas system or a guided nuclease;
optionally wherein the heterologous DNA encodes a guide RNA and/or a Cas;
B. an antibacterial agent;
C. a phage tail fibre or component thereof;
D. a vitamin;
E. a blood protein;
F. an antibody or fragment thereof; or
G. a human or plant protein or fragment thereof.

15. (canceled)

16: A method of producing synthetic phage particles, comprising
(a) allowing the production of synthetic phage in producer cells, wherein the phage are according to claim 1; and
(b) isolating the phage; and
(c) Optionally combining a population of said isolated synthetic phage with a pharmaceutically acceptable excipient, carrier or diluent to produce a pharmaceutical composition.

17: A method of producing a pharmaceutical composition, the method comprising combining a population of synthetic phage with a pharmaceutically acceptable excipient, carrier or diluent to produce a pharmaceutical composition, wherein the phages are according to claim 1.

18: A population of synthetic phage according to claim 1; optionally for administration to a human or animal subject for reducing infection by pathogenic host bacterial or archaeal cells or a first species or strain, wherein the phages are capable of infecting cells of said species or strain.

19: A synthetic phage, according to claim 1, wherein the phage is
(a) a synthetic T-even phage that comprises a deletion of DNA from, and/or an insertion of heterologous DNA into, a region of the genome of the phage corresponding to a region between coordinates
(i) 1887 and 8983;
(ii) 2625 and 8092;
(iii) 1904 and 8113;
(iv) 2668 and 7178;
(v) 7844 and 11117;
(vi) 8643 and 10313;
(vii) 9231 and 13383;
(viii) 9480 and 12224;
(ix) 8454 and 17479; or
(x) 9067 and 16673;
wherein coordinates are with reference to wild-type T4 phage genome (SEQ ID NO: 129); or
(b) a synthetic version of a phage that is not a T-even phage, wherein the synthetic phage comprises a deletion of DNA from a region of its genome that is homologous or orthologous to said region of (a);
and
wherein the synthetic phage is capable of replication in a host bacterial cell.

20: A method of producing a synthetic phage according to claim 1, the method comprising
(a) providing a heterologous DNA comprising an insert;
(b) providing a first phage genomic DNA;
(c) allowing homologous recombination between a first region of the genomic DNA and the heterologous DNA and allowing homologous recombination between a second region of the genomic DNA and the heterologous DNA,
wherein the insert is inserted between said regions whereby a hybrid DNA is produced that encodes the genome of a synthetic phage; and
wherein
A:
(i) the coordinates of the first region are 1887-2625 and the coordinates of the second region are 8092-8983;
(ii) the coordinates of the first region are 1904-2668 and the coordinates of the second region are 7178-8113;
(iii) the coordinates of the first region are 7844-8643 and the coordinates of the second region are 10313-11117;
(iv) the coordinates of the first region are 8873-9480 and the coordinates of the second region are 12224-12826; or
(v) the coordinates of the first region are 8454-9067 and the coordinates of the second region are 16673-17479;
wherein the first phage is a T4 phage and the coordinates are with reference to wild-type T4 phage genome (SEQ ID NO: 129);
or
B: the first phage is a T-even phage that is not a T4 phage, and wherein the first and second regions are regions of the first phage genome that are homologous or orthologous to said first and second regions of any one of A(i) to (v).

21: A synthetic phage obtainable by the method of claim 20.

22: A DNA comprising the genome of the synthetic phage of claim 19; optionally wherein the DNA is a chromosome of a bacterial cell or an episome comprised by a bacterial cell, such as a host cell of said synthetic phage.

23: A method of producing a modified genome of a first virus, wherein the modified genome comprises a total number (X) of base pairs of heterologous DNA, wherein the first virus is capable of infecting a target cell of a first species or strain, the method comprising
(a) obtaining sequence(s) of the genome of the first virus at least to the extent comprising a first set of genes required for virus particle production in a host cell; and
(b) producing a hybrid DNA comprising the sequence(s) obtained in step (a) and said heterologous DNA, wherein the hybrid DNA comprises said modified genome;
wherein
(c) the modified genome is functional to produce a second virus that is capable of infecting the target cell, the second virus comprising proteins encoded by said set of genes, wherein the proteins package hybrid DNA comprising said heterologous DNA and said set of genes, wherein the second virus is a modified version of the first virus; and
(d) A: the hybrid DNA excludes a total number (Y) of base pairs of DNA of the genome of the first virus wherein Y is at least 49% of X; or
B: the second virus comprises a capsid that has a DNA packaging capacity of Zbp and the total number of base pairs of the hybrid DNA is 90-110% of Z.

24: The method of claim 23, wherein
(a) each virus is a phage and the hybrid DNA excludes a DNA sequence that is comprised by a gene of the first virus genome, wherein the gene encodes an amino acid sequence selected from the group consisting of SEQ ID NOs: 1-128, or an amino acid sequence that is at least 80% identical to said amino acid sequence;
(b) the hybrid DNA excludes a plurality of DNA sequences of the first virus genome, wherein each DNA sequence is comprised by a respective gene of the first virus genome, wherein the gene encodes an amino acid sequence selected from selected from the group consisting of SEQ ID NOs: 1-128, or an amino acid sequence that is at least 80% identical to said amino acid sequence;
(c) each virus is a T even phage and the hybrid DNA excludes one or more DNA sequences of the first virus genome, wherein each DNA sequence is comprised by a respective gene of the first virus genome, wherein the gene encodes an amino acid sequence selected from the group consisting of SEQ ID NOs: 1-42, or an amino acid sequence that is at least 80% identical to said amino acid sequence;
(d) each virus is a phage and the hybrid DNA excludes one or more DNA sequences of the first virus genome, wherein each DNA sequence is comprised by at least 10% of a respective gene of the first virus genome, wherein (i) the gene encodes an amino acid sequence selected from the group consisting of SEQ ID NOs: 1-42, or an amino acid sequence that is at least 80% identical said amino acid sequence; or (ii) the gene is selected from the group consisting of T4 phage genes 49.1, 49.2, 49.3, nrdC, nrdC.1, nrdC.2, nrdC.3, nrdC.4, nrdC.5, nrdC.6, nrdC.7, nrdC.8, nrdC.9, nrdC.10, nrdC.11, mobD, mobD.1, mobD.2, mobD.2a, mobD.3, mobD.4, mobD.5, rI.-1, rI, rI.1, tk, tk.1, tk.2, tk.3, tk.4, vs, vs.1, regB, vs.3, vs.4, vs.5, vs.6, vs.7, vs.8, denV, ipIII and ipII; or a sequence that is at least 80% identical to the sequence of said gene;
(e) the hybrid DNA excludes one or more genes of the first virus genome, wherein each gene is selected from the group consisting of T4 phage genes 49.1, 49.2, 49.3, nrdC, nrdC.1, nrdC.2, nrdC.3, nrdC.4, nrdC.5, nrdC.6, nrdC.7, nrdC.8, nrdC.9, nrdC.10, nrdC.11, mobD, mobD. 1, mobD.2, mobD.2a, mobD.3, mobD.4, mobD.5, rI.-1, rI, rI.1, tk, tk.1, tk.2, tk.3, tk.4, vs, vs.1, regB, vs.3, vs.4, vs.5, vs.6, vs.7, vs.8, denV, IpIII and IpII; or a sequence that is at least 80% identical to the sequence of said gene; and/or
(f) each gene encodes a protein selected from the group consisting of a thioredoxin, endonuclease (optionally a homing endonuclease, a RegB site-specific RNA endonuclease or a site-specific intron-like DNA endonuclease), lysis inhibition regulator, membrane protein, thymidine kinase, protein that contains a A1pp phosphatase motif, tRNA synthetase modifier (optionally a valyl-tRNA synthetase modifier), mRNA processing protein, UV repair enzyme (optionally a N-glycosylase UV repair enzyme), internal head protein (eg, a IpIII internal head protein or a IpII internal head protein, Ip4 protein), endoribonuclease and DNA glycosylase (optionally a pyrimidine dimer DNA glycosylase).

25: The method of claim 23, wherein each virus comprises a life cycle having a lytic pathway, wherein (i) each virus is a lytic virus; or (ii) the first virus is a temperate virus having a life cycle comprising a lytic pathway and a lysogenic pathway, wherein the second virus has a life cycle comprising a lytic pathway but no lysogenic pathway or a disrupted lysogenic pathway wherein the second virus has a reduced chance of entering a lysogenic pathway than the first virus.

26: A method of producing synthetic virus particles, comprising carrying out the method of claim 23 to produce the hybrid DNA, introducing the hybrid DNA into a target cell of a first species or strain in which the hybrid DNA is capable of being replicated and particles of said second virus are produced; and producing second viruses in the cell; and further optionally isolating second virus particles from the cell.

27: A method of selecting a synthetic virus, the method comprising
(a) providing a first type (T1) of a virus, wherein the virus is obtained or obtainable by the method of claim 26;
(b) providing a second type (T2) of a virus, wherein the virus is obtained or obtainable by the method of claim 26, wherein T1 and T2 differ from each other by at least said heterologous DNA comprised by each type (optionally, T1 and T2 differ by heterologous DNA encoding first and second tail fibres respectively, wherein the tail fibres are different);
(c) culturing the T1 virus with target cells of the first species or strain; and culturing the T2 virus with target cells of the first species or strain;
(d) determining which of the cultured T1 and T2 viruses produces a predetermined indicator or the extent of production of the indicator by the viruses;
(e) selecting T1 or T2 virus on the basis of the determination in step (d); and
(f) Optionally further producing further copies of the selected virus and/or determining the sequence of the heterologous DNA or a portion thereof comprised by the selected virus.

28: A virus infectivity assay, the assay comprising
(a) providing a first type (T1) of virus comprising a first DNA sequence;
(b) providing a second type (T2) of virus comprising a second DNA sequence, wherein T1 and T2 differ from each other by said DNA sequences and differ in infectivity of target cells;
(c) culturing the T1 virus with target cells of a first species or strain; and culturing the T2 virus with target cells of the first species or strain;
(d) determining which of the cultured T1 and T2 viruses produces a predetermined indicator or the extent of production of the indicator by the viruses; and
(e) selecting T1 or T2 virus on the basis of the determination in step (d); and
(f) Optionally further producing further copies of the selected virus and/or determining the sequence of said DNA or a portion thereof comprised by the selected virus.

29-30. (canceled)

31: A method of producing synthetic phage particles that are capable of replication in a host cell, wherein the method comprises:
(i) deleting DNA from and/or inserting DNA into a Deletion Permissive Region (DPR) of the genome of a synthetic T-even phage, wherein the region is between the pin (protease inhibitor) gene and the iPII (internal protein) gene;
(ii) deleting DNA from a region of the genome of a synthetic version of a phage that is not a T-even phage, wherein the region is homologous or orthologous to the DPR of a synthetic T-even phage;
(iii) deleting DNA from and/or inserting DNA into a Deletion Permissive Region (DPR) of the genome of a synthetic rV5 or rV5-like phage, wherein the region is between gene 39 and gene 46 or between gene 230 and gene 240; or
(iv) deleting DNA from a region of the genome of a synthetic version of a phage that is not a rV5 or rV5-like phage, wherein the region is homologous or orthologous to the DPR of a synthetic rV5 or rV5-like phage.

32: The method of claim 31, wherein up to 8000 bp of DNA are deleted and/or inserted.

33: The method of claim 31, wherein
the inserting of (i) comprises inserting heterologous DNA into the genome of the synthetic phage, wherein the insertion is between the pin gene and the ipII gene;
the inserting of (ii) comprises inserting heterologous DNA, wherein the insertion is between a first gene and a second gene, wherein the first gene is homologous or orthologous to the pin gene of T4 and the second gene is homologous or orthologous to the ipII gene of T4;
the inserting of (iii) comprises inserting heterologous DNA into the genome of the synthetic phage, wherein the insertion is between genes 39 and 46 or between genes 230 and 240;
the inserting of (iv) comprises inserting heterologous DNA into the synthetic phage of (iv), wherein the insertion is between a first gene and a second gene, wherein the first gene is homologous or orthologous to gene 39 of Phi92 and the second gene is homologous or orthologous to gene 46 of Phi92, or wherein the first gene is homologous or orthologous to gene 230 of Phi92 and the second gene is homologous or orthologous to gene 240 of Phi92.

34: The method of claim 33, wherein the inserting comprises inserting a total number (X) of base pairs of heterologous DNA, and (a) the deleting comprises deleting a total number (Y) of base pairs of DNA wherein Y is at least 50% of X; or (b) the T-even phage, said phage that is not a T-even phage, the rV5 or rV5-like phage or the phage that is not a rV5 or rV5-like phage comprises a capsid that has a DNA packaging capacity of Zbp and the total number of base pairs of the genomic DNA of the synthetic phage is 90-110% of Z.

35: The method of claim 31, wherein
the DPR of the T-even phage comprises contiguous DNA between the pin gene and the ipII gene, wherein the contiguous DNA is at least 1000 bp in length; or wherein the DPR of the T-even phage comprises at least 100 bp of DNA between the pin gene and the ipII gene; or the DPR of the Phi92 phage comprises contiguous DNA between gene 39 and gene 46 or between gene 230 and gene 240, wherein the contiguous DNA is at least 1000 bp in length; or wherein the DPR of the Phi92 phage comprises at least 100 bp of DNA between gene 39 and gene 46 or between gene 230 and gene 240.

36: The method of claim 31, wherein
the DPR of the T-even phage extends from the pin gene to the ipII gene; or
the DPR of the Phi92 phage extends from gene 39 to gene 46 and/or from gene 230 to gene 240.

37: The method of claim 31, wherein
A. the deleting comprises deleting one or more genes from the synthetic phage, wherein each gene encodes a protein selected from a thioredoxin, endonuclease, lysis inhibition regulator, membrane protein, thymidine kinase, protein that contains a A1pp phosphatase motif, tRNA synthetase modifier, mRNA processing protein, UV repair enzyme, internal head protein, endoribonuclease and DNA glycosylase;
B. the deleting comprises deleting one, one or more, or all T4 genes of Table 7, or a sequence that is at least 80% identical to the sequence of said gene, from the synthetic phage genome;
C. the deleting comprises deleting T4 gene(s) (a) nrdC, (b) mobD, (c) rI, (d) rI.1, (e) tk, (f) vs, (g) regB and/or (h) denV, or a sequence that is at least 80% identical to the sequence of said gene, from the synthetic phage genome; or
D. the deleting comprises deleting of DNA between coordinates
a) 2625 and 8092;
b) 2668 and 7178;
c) 8643 and 10313; or
d) 9480 and 12224
from the synthetic phage genome,
wherein the coordinates are the nucleotide positions in the direction from the pin gene towards the mobD and iPII genes of T4; or wherein homologous DNA from a T-even phage is deleted wherein said T-even phage is not a T4 phage.

38: The method of claim 31, wherein the deleting comprises deleting T4 genes tk, vs and regB, or a sequence that is at least 80% identical to said gene, from the synthetic phage genome.

39: The method of claim 31, wherein the deleting comprises deleting one or more genes from the synthetic phage genome, wherein
A. each gene encodes a protein comprising an amino acid sequence selected from the group consisting of SEQ ID NOs: 1-128, or an amino acid sequence that is at least 80% identical to said amino acid sequence; and/or
B. each gene encodes an amino acid sequence selected from the group consisting of SEQ ID NOs: 1-42, or an amino acid sequence that is at least 80% identical to said amino acid sequence.

40: The synthetic phage of claim 31, wherein
(a) the deleting of (iii) or (iv) comprises deleting one or more genes from the synthetic phage genome, wherein each gene encodes a DNA methylase;
(b) the deleting of (iii) or (iv) comprises deleting one, more or all Phi92 genes of Table 9, or a sequence that is at least 80% identical to the sequence of said gene, from the synthetic phage genome;
(c) the deleting of (iii) or (iv) comprises deleting one or more Phi92 genes 235, 236, 237, 238, 239 and 240, or a sequence that is at least 80% identical to the sequence of said gene, from the synthetic phage genome; and/or
(d) the deleting of (iii) or (iv) comprises deleting Phi92 genes 39-46 and/or 235-240, or a sequence that is at least 80% identical to the sequence of said gene, from the synthetic phage genome.

41: The method of claim 31, wherein the synthetic phage is a lytic phage.

42: The method of claim 31, wherein the heterologous DNA comprises or encodes
A. one or more components of a CRISPR/Cas system or a guided nuclease;
B. an antibacterial agent;
C. a phage tail fibre or component thereof;
D. a vitamin;
E. a blood protein;
F. an antibody or fragment thereof; or
G. a human or plant protein or fragment thereof.

43: The method of claim 31, wherein said phage of (i) is selected from the group consisting of the phages of Table 6, Escherichia phage T4, Escherichia phage T2, Escherichia phage T6, Escherichia phage RB69, Shigella phage Shf125875, Escherichia phage APCEc01, Escherichia phage moskry, Escherichia phage ST0, Escherichia phage vB_EcoM_JS09, Shigella phage SP18, Escherichia phage vB_EcoM_PhAPEC2, Escherichia phage HX01, Salmonella phage SG1, Shigella phage pSs-1, Escherichia phage HY01, Yersinia phage PST, Escherichia phage AR1, Escherichia phage phiE142, Shigella phage SHFML-11, Escherichia phage slur07, Shigella phage SHFML-11, Escherichia phage UFV-AREG1, Escherichia phage vB_EcoM-UFV13, Shigella phage JK38, Shigella phage SHFML-26, Shigella phage Sf22, Escherichia phage ime09, Shigella phage SH7, Yersinia phage phiD1, Escherichia phage RB3, Escherichia phage ECML-134, Escherichia phage vB_EcoM_ACG-C40, Escherichia phage vB_EcoM-fFiEco06, Escherichia phage PP01, Shigella phage Shf12, Escherichia phage ECO4, Escherichia virus RB14, Escherichia phage vB_EcoM_JB75, Shigella phage Sf22, Escherichia phage vB_vPM_PD112, Shigella phage Sf23, Escherichia phage vB_EcoM_G2540, Escherichia phage vB_EcoM_G2133, Escherichia phage vB_EcoM_G4498, Escherichia virus RB32, Escherichia phage vB_EcoM_G4507, Escherichia phage vB_EcoM_G8, Escherichia phage EcNP 1, Enterobacteria phage RB27, Shigella virus KRT47, Escherichia phage teqdroes, Escherichia phage slur02, Yersinia phage fPS-90, Yersinia phage phiD1, Shigella phage Sf24 and Escherichia phage phiC120.
